(19) United States
(12) Patent Application Publication
(10) Pub. No.: US 2023/0100838 A1
(43) Pub. Date: Mar. 30, 2023

(54) COVALENT RAS INHIBITORS AND USES THEREOF

(71) Applicant: Revolution Medicines, Inc., Redwood City, CA (US)

(72) Inventors: Jennifer PITZEN, Fremont, CA (US); James AGGEN, Burlingame, CA (US); G. Leslie BURNETT, Redwood City, CA (US); Adrian L. GILL, Atherton, CA (US); Christopher SEMKO, Fremont, CA (US); Anne V. EDWARDS, San Mateo, CA (US); Micah James GLIEDT, Fremont, CA (US); Gert KISS, Palo Alto, CA (US); Ashutosh JOGALEKAR, Menlo Park, CA (US); John E. KNOX, Emerald Hills, CA (US); Andreas BUCKL, San Francisco, CA (US); Elena S. KOLTUN, Foster City, CA (US)

(21) Appl. No.: 17/825,875

(22) Filed: May 26, 2022

Related U.S. Application Data
(63) Continuation of application No. PCT/US2020/062391, filed on Nov. 25, 2020.
(60) Provisional application No. 63/024,868, filed on May 14, 2020.
(60) Provisional application No. 62/969,415, filed on Feb. 3, 2020.
(60) Provisional application No. 62/940,947, filed on Nov. 27, 2019.

(51) Int. Cl. A61K 47/64; A61K 45/06; C07D 471/04; C07D 401/12; C07D 401/04; C07D 239/94; C07D 413/14; C07D 498/16

(52) U.S. Cl. CPC A61K 47/64; A61K 45/06; C07D 471/04; C07D 401/12; C07D 401/04; C07D 239/94; C07D 413/14; C07D 498/16

(57) ABSTRACT

The disclosure features compounds, or pharmaceutically acceptable salts thereof, alone and in combination with other therapeutic agents, pharmaceutical compositions, and protein conjugates thereof, capable of modulating biological processes including Ras, and their uses in the treatment of cancers.

### CROSS-REFERENCE TO RELATED APPLICATIONS

The present application is a continuation of International Application No. PCT/US2020/062391, filed on Nov. 25, 2022, which claims the benefit of priority to U.S. Application No. 62/940,947, filed on Nov. 27, 2019; U.S. Application No. 62/969,415, filed on Feb. 3, 2020; and U.S. Application No. 63/024,868, filed on May 14, 2020, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on May 26, 2022, is named 51432-010001_Sequence Listing_5_26_22_ST25 and is 5,412 bytes in size.

### BACKGROUND OF THE INVENTION

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18(8): 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras and N-Ras) play an essential role in various human cancers and are therefore appropriate targets for anticancer therapy. Dysregulation of Ras proteins by activating mutations, overexpression or upstream activation is common in human tumors, and activating mutations in Ras are frequently found in human cancer. See, e.g., Prior et al., Cancer Res 72(10): 2457-2467 (2012). Of the Ras proteins, K-Ras is the most frequently mutated and is therefore an important target for cancer therapy. Despite extensive small molecule drug discovery efforts against Ras during the last several decades, a drug directly targeting Ras is still not available for clinical use. However, the reputation of the “undruggability” of Ras proteins by small molecules has been challenged of late (see, e.g., Ostrem et al., Nature 503(7477), 548-551 (2013). Additional efforts are needed to uncover new medical treatments for cancers driven by Ras mutations, such as by identifying new small molecule Ras inhibitors.

### SUMMARY OF THE INVENTION

Covalent drugs bond covalently to their biological target. Covalent drugs have a long history in medicine and will continue to impact drug discovery and human health into the future. Biological targets with nucleophilic functional groups such as —SH, —OH, —NH₂, —COOH and others are potentially amenable to a covalent drug discovery approach. For example, the irreversibly covalent drug ibrutinib was approved by the FDA in 2013 for the treatment of mantle cell lymphoma, and its label has since been expanded.

Provided herein are compounds which are capable of binding to a Ras protein to form a conjugate by reacting as electrophiles and forming a covalent bond with a nucleophilic Ras amino acid of a Ras protein. Conjugate formation via covalent binding of a compound of the present invention may disrupt downstream signaling of Ras. The Ras protein may be wild type or a mutant Ras protein. The amino acid may, for example, be an aspartic acid, a serine, or a cysteine of a Ras protein. In some embodiments, compounds of the invention form a covalent bond with an aspartic acid, a serine, or a cysteine at the 12 position of a mutant K-Ras, H-Ras or N-Ras protein. In some embodiments, compounds disclosed herein form a covalent bond with the aspartic acid residue at position 12 of K-Ras G12D. In some embodiments, compounds disclosed herein form a covalent bond with the aspartic acid residue at position 13 of K-Ras G13D. In some embodiments, compounds disclosed herein form a covalent bond with the serine residue at position 12 of K-Ras G12S. In some embodiments, a compound of the present invention may be useful in the treatment of diseases and disorders in which Ras, particularly mutated Ras, play a role, such as cancer. Additional aspects of the foregoing are further described herein.

Accordingly, provided herein is a compound having the structure of Formula I:

A-L-B Formula I

wherein A is a Ras binding moiety; L is a linker; and B is a selective cross-linking group, or a pharmaceutically acceptable salt thereof, wherein, upon contacting the compound, or a pharmaceutically acceptable salt thereof, with a sample containing a Ras protein, at least 20% of the Ras protein in the sample covalently reacts with the compound, or a pharmaceutically acceptable salt thereof, to form a conjugate.

Also provided is a pharmaceutical composition comprising a compound of the present invention, or a pharmaceutically acceptable salt thereof, and a pharmaceutically acceptable excipient.

Further provided is a conjugate, or salt thereof, comprising a Ras protein covalently bound to a selective cross-linking group, which selective cross-linking group is bound to a Ras binding moiety through a linker, wherein the selective cross-linking group is a carbodiimide, an aminooxazoline, a chloroethyl urea, an aziridine, a trifluoromethyl ketone, a boronic acid, a boronic ester, an N-ethoxycarbonyl-2-ethoxy-1,2-dihydroquinoline (EEDQ), an iso-EEDQ, an epoxide, an oxazolium, or a glycal.

Further provided is a Ras protein comprising a covalent bond to a compound of the present invention. In some embodiments, an inhibited Ras protein covalently bonded to a compound of the present invention is provided. In some embodiments, a wild-type Ras protein covalently bonded to a compound of the present invention is provided. In some embodiments, a mutated Ras protein covalently bonded to a compound of the present invention is provided.

Also provided is a method of producing a conjugate comprising contacting a Ras protein with a compound of the present invention, or a pharmaceutically acceptable salt thereof, or a pharmaceutical composition comprising such a compound or salt, under conditions sufficient for the compound to react covalently with the Ras protein, or under conditions suitable to permit conjugate formation. Conjugates produced by such methods are also provided.

Further provided is a method of treating cancer in a subject in need thereof, the method comprising administering to the subject a therapeutically effective amount of a compound of the present invention, or a pharmaceutically acceptable salt thereof, or a pharmaceutical composition comprising such a compound or salt.

Also provided is a method of inhibiting a Ras protein in a cell, the method comprising contacting the cell with an effective amount of a compound of the present invention, or a pharmaceutically acceptable salt thereof, or a pharmaceutical composition comprising such a compound or salt.

In some embodiments, a method of treating a Ras protein-related disorder in a subject in need thereof is provided, the method comprising administering to the subject a therapeutically effective of a compound of the present invention, or a pharmaceutically acceptable salt thereof, or a pharmaceutical composition comprising such a compound or salt.

### DETAILED DESCRIPTION OF THE INVENTION

### Definitions

In this application, unless otherwise clear from context, (i) the term “a” is understood to mean “at least one”; (ii) the term “or” is understood to mean “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value.

As used herein, the term “adjacent” in the context of describing adjacent atoms refers to bivalent atoms that are directly connected by a covalent bond.

It will be understood that the term “binding” as used herein, typically refers to association (e.g., non-covalent or covalent, hydrogen bonds, van der Waals interaction, hydrophobic interactions, magnetism, and combinations thereof) between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity or in a biological system or cell).

As used herein, the term “corresponding to” is often used to designate a structural element or moiety in a compound of interest that shares a position (e.g., in three-dimensional space or relative to another element or moiety) with one present in an appropriate reference compound. For example, in some embodiments, the term is used to refer to position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190^{th} residue in the first polymer but rather corresponds to the residue found at the 190^{th} position in the reference polymer; those of ordinary skill in the art readily appreciate how to identify “corresponding” amino acids, including through use of one or more commercially-available algorithms specifically designed for polymer sequence comparisons.

As used herein, the term “inhibitor” refers to a compound that i) inhibits, decreases or reduces the effects of a protein, such as a Ras protein; or ii) inhibits, decreases, reduces, or delays one or more biological events. The term “inhibiting” or any variation thereof, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity (e.g., Ras activity) compared to normal.

The term “pure” means substantially pure or free of unwanted components (e.g., other compounds), material defilement, admixture or imperfection.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Those skilled in the art will appreciate that, in some embodiments, isotopes of compounds described herein may be prepared or utilized in accordance with the present invention. “Isotopes” refers to atoms having the same atomic number but different mass numbers resulting from a different number of neutrons in the nuclei. For example, isotopes of hydrogen include tritium and deuterium. Other isotopes include, e.g., ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I. In some embodiments, an isotopic substitution (e.g., substitution of hydrogen with deuterium) may alter the physicochemical properties of the molecules, such as metabolism, the distribution of metabolites, or the rate of racemization of a chiral center. Methods of incorporating one or more of such isotopes into compounds are known to those of skill in the art.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocyclyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘};

—N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R^{∘})—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —O C(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)N R^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}, —Si R^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, —C₁₋₆ aliphatic, —CH₂Ph, —O(CH₂)₀₋₁Ph,
—CH₂-(5-6 membered heteroaryl ring), or a 3-8-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen,

—(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-8-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-8-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-8-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-8-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-8-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-8-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein for an alkyl group.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, 2-butenyl, and the like. Alkenyls include both cis and trans isomers. The term “alkenylene,” as used herein represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, 1-propynyl, and the like.

The term “amino,” as used herein, represents —N(R^{†})₂.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in is broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- or amino-terminal amino acid in a polypeptide, can contain a structural modification as compared with the general structure above. For example, in some embodiments, an amino acid may be modified by methylation, amidation, acetylation, or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide. In some embodiments, the amino acid is attached to the parent molecular group by a carbonyl group, where the side chain or amino group is attached to the carbonyl group. In some embodiments, the amino acid is an α-amino acid. In certain embodiments, the amino acid is a β-amino acid. In some embodiments, the amino acid is a γ-amino acid. Exemplary side chains include an optionally substituted alkyl, aryl, heterocyclyl, alkaryl, alkheterocyclyl, aminoalkyl, carbamoylalkyl, and carboxyalkyl. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, omithine, phenylalanine, proline, pyrrolysine, selenocysteine, serine, taurine, threonine, tryptophan, tyrosine, and valine.

The term “aryl,” as used herein, represents a monovalent mono-, bicyclic, or multicyclic ring system formed by carbon atoms, wherein each ring is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH.

The term “cyano,” as used herein, represents a —CN group.

The term “cycloalkyl,” as used herein, represents a monovalent saturated cyclic hydrocarbon group, which may be bridged, fused or spirocyclic, which may be fused, having from three to eight ring carbons, unless otherwise specified, and is exemplified by cyclopropyl, cyclobutyl, cyclopentyl, cyclohexyl, cycloheptyl, cycloheptyl, and the like.

The term “diyl,” when used in the name of a chemical compound represents a divalent radical.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “halo,” as used herein, represents a halogen selected from bromine, chlorine, iodine, or fluorine.

The term “heteroalkyl” as used herein refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, isoquinolinyl, tetrahydroquinolinyl, 4-azaindolyl, or and the like. A heteroaryl ring can be attached to its pendant group at any ring atom that results in a stable structure and any of the ring atoms can be optionally substituted unless otherwise specified. In some embodiment, the heteroaryl is substituted with 1, 2, 3, or 4 substituents groups.

The term “heterocyclyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocyclyl groups are of 1 to 12 (e.g., 1 to 11, 1 to 10, 1 to 9, 2 to 12, 2 to 11, 2 to 10, or 2 to 9) carbons. The term “heterocyclyl” also represents a heterocyclic compound having a bridged multicyclic structure in which one or more carbons or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocyclyl groups are pyrrolidinyl, piperidinyl, 1,2,3,4-tetrahydroquinolinyl, decahydroquinolinyl, dihydropyrrolopyridine, decahydronapthyridinyl, or and the like. A heterocyclic ring can be attached to its pendant group at any ring atom that results in a stable structure and any of the ring atoms can be optionally substituted unless otherwise specified.

The term “haloalkyl,” as used herein, represents an alkyl moiety substituted on one or more carbon atoms with one or more of the same of different halo moieties.

The term “hydroxyalkyl,” as used herein, represents an alkyl moiety substituted on one or more carbon atoms with one or more —OH moieties.

The term “isomer,” as used herein, means any tautomer, stereoisomer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “nitro,” as used herein, represents a —NO₂ group.

The term “oxo” as used herein, represents ═O.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure. Some compounds of the present invention may exist in different tautomeric forms, all of the latter being included within the scope of the present invention.

The term “sulfonyl,” as used herein, represents an —S(O)₂— group.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form; etc.

The term “Ras protein” means a protein from the Ras family of related GTPase proteins including K-Ras, H-Ras, and N-Ras. A Ras protein may be a wild-type protein or a mutant protein. In some embodiments, a Ras protein is not a wild-type protein.

K-Ras is encoded by the K-RAS gene. The term “K-Ras” also refers to natural variants of the wild-type K-Ras protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type K-Ras, which is set forth in SEQ ID NO: 1.

| SEQ ID NO: 1 | |
|---|---|
| | MTEYKLVVVG AGGVGKSALT IQLIQNHFVD EYDPTIEDSY |
| | RKQVVIDGET CLLDILDTAG QEEYSAMRDQ YMRTGEGFLC |
| | VFAINNTKSF EDIHHYREQI KRVKDSEDVP MVLVGNKCDL |
| | PSRTVDTKQA QDLARSYGIP FIETSAKTRQ PVEDAFYTLV |
| | REIRQYPLKK ISKEEKTPGC VKIKKCIIM |

H-Ras is encoded by the H-RAS gene. The term “H-Ras” also refers to natural variants of the wild-type H-Ras protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type H-Ras, which is set forth in SEQ ID NO: 2.

SEQ ID NO: 2

MTEYKLVVVG AGGVGKSALT IQLIQNHFVD EYDPTIEDSY
RKQVVIDGET CLLDILDTAG QEEYSAMRDQ YMRTGEGFLC
VFAINNTKSF EDIHQYREQI KRVKDSDDVP MVLVGNKCDL
AARYVESRQA QDLARSYGIP YIETSAKTRQ GVFDAFYTLV
REIRQHKLRK LNPPDESGPG CMSCKCVLS

N-Ras is encoded by the N-RAS gene. The term “N-Ras” also refers to natural variants of the wild-type N-Ras protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type N-Ras, which is set forth in SEQ ID NO: 3.

SEQ ID NO: 3

MTEYKLVVVG AGGVGKSALT IQLIQNHFVD EYDPTIEDSY
RKQVVIDGET CLLDILDTAG QEEYSAMRDQ YMRTGEGFLC
VFAINNSKSF ADINLYPEQI KRVKDSDDVP MVLVGNKCDL
PTRTVDTKQA HELAKSYGIP FIETSAKTRQ GVEDAFYTLV
REIRQYRMKK LNSSDDGTQG CMGLPCVVM

A given Ras protein may be bound to GDP or GTP. In response to exposure of the cell to certain growth promoting stimuli, RAS is induced to exchange its bound GDP for a GTP. With GTP bound, RAS is “switched on” and is able to interact with and activate other proteins (its “downstream targets”). Ras itself has a very low intrinsic ability to hydrolyze GTP back to GDP, thus turning itself into the off state. Switching R as off requires extrinsic proteins termed GTPase-activating proteins (GAPs) that interact with RAS and greatly accelerate the conversion of GTP to GDP. Any mutation in Ras which affects its ability to interact with GAP or to convert GTP back to GDP will result in a prolonged activation of the protein and consequently a prolonged signal to the cell telling it to continue to grow and divide. Because these signals result in cell growth and division, overactive RAS signaling may ultimately lead to cancer. Methods of determining the GDP or GTP binding state of a Ras protein are known in the art.

As used herein, the term “mutant Ras protein” means a Ras protein that comprises at least one mutation in which an amino acid in the corresponding wild-type Ras protein is mutated to a different amino acid, e.g., a glycine is mutated to an aspartic acid, serine, or cysteine. The term “mutation” as used herein indicates any modification of a nucleic acid or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequence, as well as amplifications or chromosomal breaks or translocations.

Examples of mutant Ras proteins include, but are not limited to, K-Ras G12D, K-Ras G13D, and K-Ras G12S. In some embodiments, mutations contemplated by the present invention include those associated with oncogenic activity. In some embodiments, mutations contemplated by the present invention include:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V141, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T501, A146V, or A59T, and combinations thereof.

### Compounds and Conjugates of the Present Invention

Provided herein are compounds which are capable of binding to a Ras protein to form a conjugate by reacting as electrophiles and forming a covalent bond with a nucleophilic Ras amino acid of a Ras protein. In some embodiments, a compound of the present invention may be useful in the treatment of diseases and disorders in which Ras, particularly mutated Ras, play a role, such as cancer. Compounds described or depicted herein, whether expressly stated or not, may be provided or utilized in salt form, e.g., a pharmaceutically acceptable salt form, unless expressly stated to the contrary.

Covalent binding of a compound of the present invention to Ras can be reversible or irreversible. Irreversible covalent binding to GDP-bound Ras or GTP-bound Ras can be determined by methods known to those skilled in the art, for example by mass spectrometry. For example, to determine binding to GTP or GDP-Ras, a compound of the present invention may be incubated with Ras loaded with the appropriate nucleotide, then cross-linking is determined by mass spectrometry. An example protocol is provided in the Examples below.

Moreover, covalent binding of a compound of the present invention to Ras may perturb the conformation of Ras such that it modulates or disrupts binding of Ras to its effector proteins (including SOS and RAF). Ras-RAF disruption assays are known by those skilled in the art, as described for example by Lim et al., Angew. Chem. Int. Ed. 53:199 (2014). By disrupting Ras binding to its effector proteins, compounds may disrupt downstream signaling, resulting in growth inhibition or the induction of apoptosis. These effects can be measured in cell culture following compound treatment by monitoring the activation state of downstream effectors (such as the phosphorylation state of ERK), performing a cellular viability assay, and by measuring the activity of caspase-3 in a cell lysate.

Some compounds disclosed herein may form reversible covalent bonds with Ras, including boronic acids and trifluoromethyl ketones. Boronic acids are known to interact with seine and threonine residues, as described for example by Adams et al., Cancer Invest. 22:304 (2004). By extension, aspartate residues could also form reversible covalent bonds with boronic acids or other electrophiles such as trifluoromethyl ketones.

Accordingly, the present disclosure features compounds of Formula I:

A-L-B Formula I

wherein A is a Ras binding moiety;

L is a linker; and

B is a selective cross-linking group,

or a pharmaceutically acceptable salt thereof. In some embodiments, upon contacting the compound, or a pharmaceutically acceptable salt thereof, with sample containing a Ras protein, at least 20% of the Ras protein in the sample covalently reacts with the compound, or a pharmaceutically acceptable salt thereof, to form a conjugate. In some embodiments, upon contacting the compound, or a pharmaceutically acceptable salt thereof, with a sample containing a Ras protein, at least 20% (e.g., at least 30%, at least 40%, at least 50%, at least 60%, at least 70%, at least 80%, at least 90%, at least 95%, at least 99%) of the Ras protein in the sample covalently reacts (e.g., forms a conjugate including the Ras binding moiety, the linker, and the Ras protein) with the compound, or a pharmaceutically acceptable salt thereof, to form a conjugate.

Ras proteins are described herein. Accordingly, a Ras protein may be wild-type or mutant. A Ras protein may be a human Ras protein. A wild-type Ras protein may be K-Ras, H-Ras, or N-Ras. In some embodiments, a Ras protein is not a wild-type protein. In some embodiments, a Ras protein is a mutant Ras protein, such as K-Ras G12D, K-Ras G13D, or K-Ras G12S. Other Ras mutants are described herein. In some embodiments, the sample containing Ras protein is a sample including isolated Ras protein in a solution, e.g., a buffer solution. In some embodiments, the sample containing Ras protein is a sample including cells expressing Ras protein.

Compounds, or a pharmaceutically acceptable salt thereof, of the invention include a Ras binding moiety. As used herein, a “Ras binding moiety” refers to a moiety that binds to a Ras protein. In some embodiments, the Ras binding moiety includes a group of atoms (e.g., 5 to 20 atoms, 5 to 10 atoms, 10 to 20 atoms, 20 to 30 atoms, 30 to 40 atoms) that bind to the Ras protein. In some embodiments, one or more atoms of a Ras binding moiety do not bind to the Ras protein.

A Ras protein can bind to a single atom in a Ras binding moiety. Alternatively, or additionally, a Ras protein can bind to two or more atoms in a Ras binding moiety. In another alternative, a Ras protein binds to a group that mimics a natural ligand of a Ras protein and wherein the group that mimics a natural ligand of a Ras protein is attached to a Ras binding moiety. Binding in these examples is typically through, but not limited to, non-covalent interactions of a Ras protein to a Ras binding moiety.

In some embodiments, the Ras binding moiety binds to the GDP-bound form of the Ras protein. In some embodiments, the Ras binding moiety binds to the GTP-bound form of the Ras protein. In some embodiments, the Ras binding moiety binds to the GDP-bound form and the GTP-bound form of the Ras protein.

In some embodiments, the Ras binding moiety is a human H-Ras binding moiety, a human N-Ras binding moiety, or a human K-Ras binding moiety. In some embodiments, the Ras binding moiety is a K-Ras binding moiety. In some embodiments, the K-Ras binding moiety binds to a residue of a K-Ras Switch-II binding pocket of the K-Ras protein, e.g., a residue of the K-Ras protein corresponding to V7, V8, V9, G10, A11, D12, K16, P34, T58, A59, G60, Q61, E62, E63, Y64, S65, R68, D69, Y71, M72, F78, I92, H95, Y96, Q99, I100, R102, or V103 of human wild-type K-Ras (SEQ ID NO: 1). In some embodiments, the Ras binding moiety is an H-Ras binding moiety that binds to a residue of an H-Ras Switch-II binding pocket of an H-Ras protein. In some embodiments, the Ras binding moiety is an N-Ras binding moiety that binds to a residue of an N-Ras Switch-II binding pocket of an N-Ras protein.

In some embodiments, the Ras binding moiety comprises the structure of any one of Formula II to V, described below.

In some embodiments, the Ras binding moiety (e.g., K-Ras binding moiety) includes the structure of Formula II:

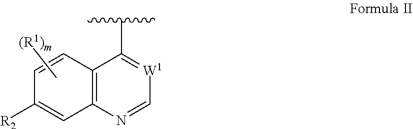

wherein m is 0, 1, 2, or 3;

W¹ is N or C, wherein C is optionally attached to the linker via an optionally substituted C₁-C₃ alkylene bridge or optionally substituted C₁-C₃ heteroalkylene bridge;

each R¹ is, independently, CN, halo, hydroxy, optionally substituted C₁-C₆ alkyl, or optionally substituted C₁-C₆ heteroalkyl, or

R¹ is attached to the linker via a C₁-C₃ alkylene bridge or C₁-C₃ heteroalkylene bridge; and

R² is optionally substituted C₆-C₁₀ aryl or optionally substituted C₂-C₉ heteroaryl.

In some embodiments of Formula II, W¹ is N or C, wherein C is attached to the linker via an optionally substituted C₁-C₃ alkylene bridge or optionally substituted C₁-C₃ heteroalkylene bridge.

In some embodiments, the Ras binding moiety (e.g., K-Ras binding moiety) includes the structure of Formula II-1:

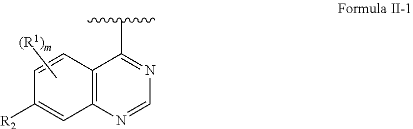

wherein m is 0, 1, 2, or 3;

each R¹ is, independently, CN, halo, hydroxy, optionally substituted C₁-C₆ alkyl, or optionally substituted C₁-C₆ heteroalkyl, or

R¹ is attached to the linker via a C₁-C₃ alkylene bridge or C₁-C₃ heteroalkylene bridge; and

R² is optionally substituted C₆-C₁₀ aryl or optionally substituted C₂-C₉ heteroaryl,

or a pharmaceutically acceptable sat thereof.

In some embodiments, the Ras binding moiety (e.g., K-Ras binding moiety) includes the structure:

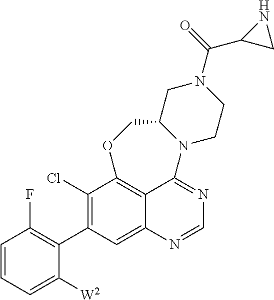

wherein W² is hydrogen or hydroxy.

In some embodiments, the Ras binding moiety (e.g., K-Ras binding moiety) includes the structure of Formula II-1a:

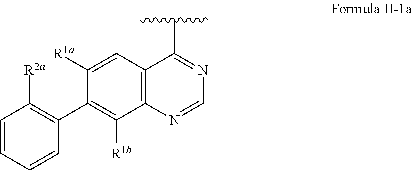

wherein R^{1a}, R^{1b}, and R^{2a} are, independently, hydrogen, CN, halo, hydroxy, optionally substituted C₁-C₆ alkyl, or optionally substituted C₁-C₆ heteroalkyl,

or a pharmaceutically acceptable salt thereof.

In some embodiments, R^{1a} is halo (e.g., chloro). In some embodiments, R^{1b} is halo (e.g., fluoro). In some embodiments, R^{2a} is halo (e.g., fluoro).

In some embodiments, the Ras binding moiety (e.g., K-Ras binding moiety) includes the structure:

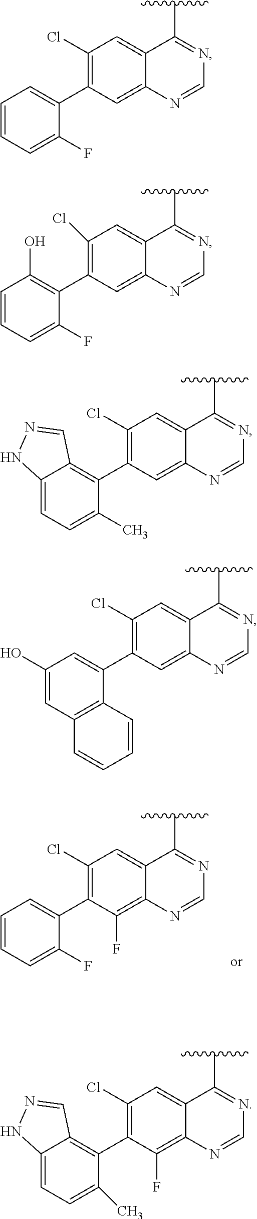 [SMILES: *c1ncnc2cc(-c3ccccc3F)c(Cl)cc12 ; *c1ncnc2cc(-c3c(O)cccc3F)c(Cl)cc12 ; *c1ncnc2cc(-c3c(C)ccc4[nH]ncc34)c(Cl)cc12 ; *c1ncnc2cc(-c3cc(O)cc4ccccc34)c(Cl)cc12 ; *c1ncnc2c(F)c(-c3ccccc3F)c(Cl)cc12 ; *c1ncnc2c(F)c(-c3c(C)ccc4[nH]ncc34)c(Cl)cc12]

In some embodiments, the Ras binding moiety (e.g., K-Ras binding moiety) includes the structure:

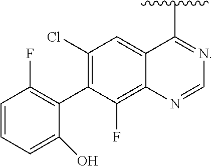 [SMILES: *c1ncnc2c(F)c(-c3c(O)cccc3F)c(Cl)cc12]

In some embodiments, the Ras binding moiety (e.g., K-Ras binding moiety) includes the structure of Formula II-2:

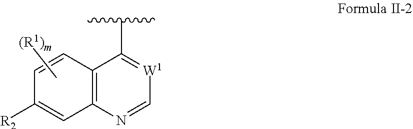

wherein m is 0, 1, 2, or 3;

W¹ is C attached to the linker via an optionally substituted C₁-C₃ alkylene bridge or an optionally substituted C₁-C₃ heteroalkylene bridge;

each R¹ is, independently, CN, halo, hydroxy, optionally substituted C₁-C₆ alkyl, or optionally substituted C₁-C₆ heteroalkyl, or

R¹ is attached to the linker via a C₁-C₃ alkylene bridge or C₁-C₃ heteroalkylene bridge; and

R² is optionally substituted C₆-C₁₀ aryl or optionally substituted C₂-C₉ heteroaryl.

In some embodiments, the Ras binding moiety (e.g., K-Ras binding moiety) includes the structure of Formula III:

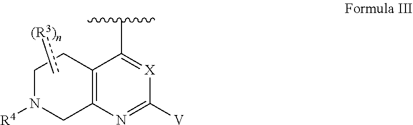

wherein n is 0, 1, 2, 3, 4, 5, or 6;

[symbol] represents a single bond or a double bond;

X is N or CR′, wherein R′ is hydrogen, or R′ is attached to the linker via an optionally substituted C₁-C₃alkylene bridge, or optionally substituted C₁-C₃ heteroalkylene bridge;

V is CHR⁵, CR⁵R⁵, OR⁵, NHR⁵, or NR^{5a}R^{5b};

each R³ is, independently,

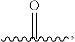 [SMILES: *=O]

optionally substituted C₁-C₆ alkyl, or optionally substituted C₁-C₆ heteroalkyl, or

R³ is attached to the linker via an optionally substituted C₁-C₃ alkylene bridge or optionally substituted C₁-C₃ heteroalkylene bridge;

R⁴ is optionally substituted C₆-C₁₀ aryl or optionally substituted C₂-C₉ heteroaryl;

each R⁵ is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl; and

each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl, or

R^{5a} and R^{5b}, together with the nitrogen atom to which each is attached, combine to form optionally substituted C₂-C₉ heterocyclyl;

provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge, and

further provided that when R³ is attached to the linker via an optionally substituted C₁-C₃ alkylene bridge or optionally substituted C₁-C₃ heteroalkylene bridge, R′ not is attached to the linker via an optionally substituted C₁-C₃ alkylene bridge or optionally substituted C₁-C₃ heteroalkylene bridge.

In some embodiments, the Ras binding moiety (e.g., K-Ras binding moiety) includes the structure of Formula III-1:

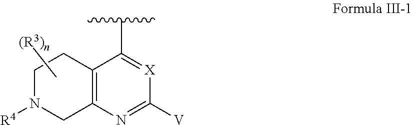

wherein n is 0, 1, 2, 3, 4, 5, or 6;

X is N or CR′, wherein R′ is hydrogen, or R′ is attached to the linker via an optionally substituted C₁-C₃ alkylene bridge or optionally substituted C₁-C₃ heteroalkylene bridge;

V is CHR⁵, CR⁵R⁵, OR⁵, NHR⁵, or NR^{5a}R^{5b};

each R³ is, independently, optionally substituted C₁-C₆ alkyl or optionally substituted C₁-C₆ heteroalkyl, or

R³ is attached to the linker via an optionally substituted C₁-C₃ alkylene bridge or optionally substituted C₁-C₃ heteroalkylene bridge;

R⁴ is optionally substituted C₆-C₁₀ aryl or optionally substituted C₂-C₉ heteroaryl;

each R⁵ is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl; and

each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl;

provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge, and further provided that when R³ is attached to the linker via an optionally substituted C₁-C₃ alkylene bridge or optionally substituted C₁-C₃ heteroalkylene bridge, R′ not is attached to the linker via an optionally substituted C₁-C₃ alkylene bridge or optionally substituted C₁-C₃ heteroalkylene bridge.

In some embodiments, the Ras binding moiety (e.g., K-Ras binding moiety) includes the structure of Formula III-1a:

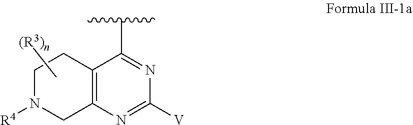

wherein n is 0, 1, 2, 3, 4, 5, or 6;

V is CHR⁵, CR⁵R⁵, OR⁵, or NHR⁵, or NR^{5a}R^{5b};

each R³ is, independently, optionally substituted C₁-C₆ alkyl or optionally substituted C₁-C₆ heteroalkyl, or

R³ is attached to the linker via a C₁-C₃ alkylene bridge or C₁-C₃ heteroalkylene bridge;

R⁴ is optionally substituted C₆-C₁₀ aryl or optionally substituted C₂-C₉ heteroaryl;

each R⁵ is, independently, optionally substituted C₁-C₆ alkyl, optionally substituted C₁-C₆ heteroalkyl, optionally substituted C₁-C₆ alkyl-C₂-C₉ heteroaryl, or optionally substituted C₁-C₆ alkyl-C₂-C₉ heterocyclyl; and

each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl.

In some embodiments, the Ras binding moiety (e.g., K-Ras binding moiety) includes the structure of Formula III-1b:

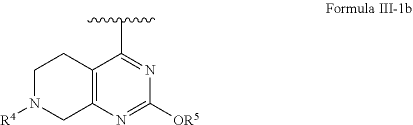

wherein R⁴ is optionally substituted C₆-C₁₀ bicyclic aryl; and

R⁵ is optionally substituted C₁-C₆ alkyl-C₂-C₉ heteroaryl or optionally substituted C₁-C₆ alkyl-C₂-C₉ heterocyclyl.

In some embodiments, the Ras binding moiety (e.g., K-Ras binding moiety) includes the structure of Formula III-2:

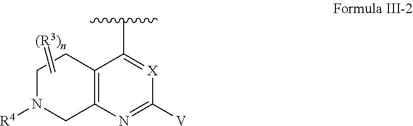

wherein n is 0, 1, 2, or 3;

X is N or CR′, wherein R′ is hydrogen, or R′ is attached to the linker via an optionally substituted C₁-C₃alkylene bridge or optionally substituted C₁-C₃ heteroalkylene bridge;

V is CHR⁵, CR⁵R⁵, OR⁵, NHR⁵, or NR^{5a}R^{5b};

each R³ is

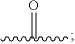 [SMILES: *=O]

R⁴ is optionally substituted C₆-C₁₀ aryl or optionally substituted C₂-C₉ heteroaryl;

each R⁵ is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl; and

each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl, or

R^{5a} and R^{5b}, together with the nitrogen atom to which each is attached, combine to form optionally substituted C₂-C₉ heterocyclyl;

provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge.

In some embodiments, the Ras the Ras binding moiety (e.g., K-Ras binding moiety) includes the structure of Formula III-2a:

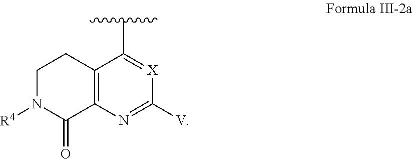

In some embodiments, the Ras binding moiety (e.g., K-Ras binding moiety) includes the structure of Formula III-3:

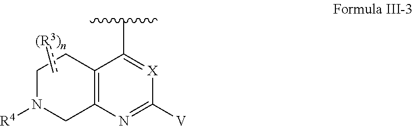

wherein n is 0, 1, 2, 3, 4, 5, or 6;

[symbol] represents a single bond or a double bond;

X is N or CR′, wherein R′ is hydrogen, or R′ is attached to the linker via an optionally substituted C₁-C₃alkylene bridge or optionally substituted C₁-C₃ heteroalkylene bridge;

V is NR^{5a}R^{5b};

each R³ is, independently,

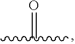 [SMILES: *=O]

optionally substituted C₁-C₆ alkyl, or optionally substituted C₁-C₆ heteroalkyl, or

R³ is attached to the linker via an optionally substituted C₁-C₃ alkylene bridge or optionally substituted C₁-C₃ heteroalkylene bridge;

R⁴ is optionally substituted C₆-C₁₀ aryl or optionally substituted C₂-C₉ heteroaryl;

R^{5a} and R^{5b}, together with the nitrogen atom to which each is attached, combine to form optionally substituted C₂-C₉ heterocyclyl;

provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge, and

further provided that when R³ is attached to the linker via an optionally substituted C₁-C₃ alkylene bridge or optionally substituted C₁-C₃ heteroalkylene bridge, R′ not is attached to the linker via an optionally substituted C₁-C₃ alkylene bridge or optionally substituted C₁-C₃ heteroalkylene bridge.

In some embodiments, R⁴ is:

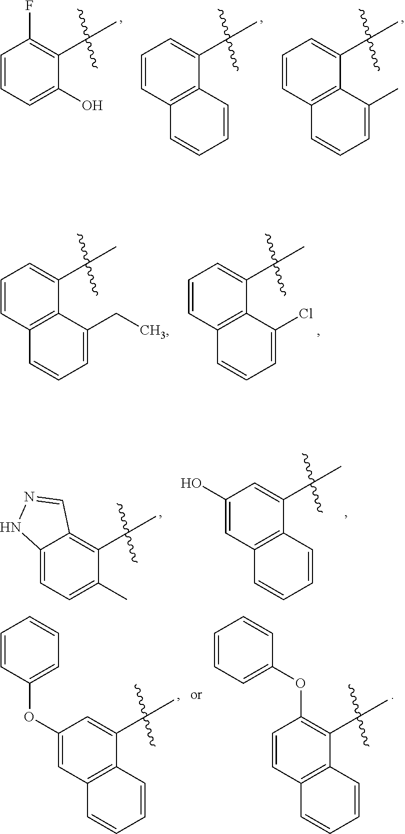

In some embodiments, R⁴ is:

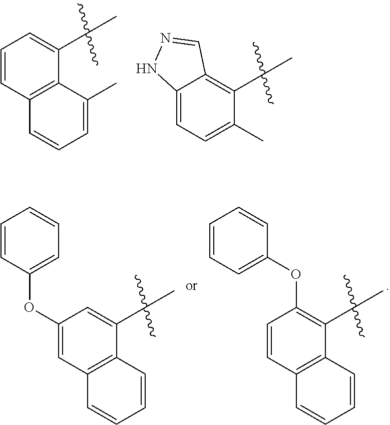

In some embodiments, R⁴ is:

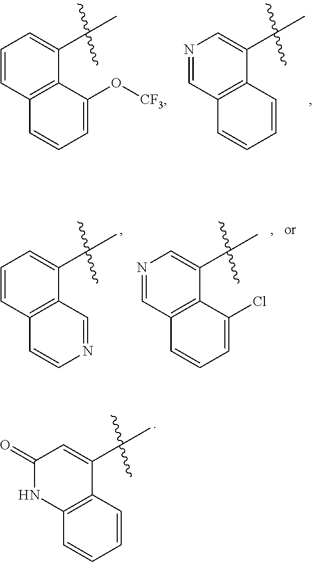

In some embodiments, V is CHR⁵ or CR⁵R⁵. In some embodiments, V is OR⁵, NHR⁵, or NR^{5a}R^{5b}. In some embodiments, V is OR⁵. In some embodiments, V is OR⁵, wherein R⁵ is optionally substituted C₁-C₆ alkyl or optionally substituted C₁-C₆ heteroalkyl. In some embodiments, V is OR⁵, wherein R⁵ is optionally substituted —C₁-C₆ alkyl-C₂-C₉ heteroaryl or optionally substituted —C₁-C₆ alkyl-C₂-C₉ heterocyclyl. In some embodiments, V is NHR⁵ or NR^{5a}R^{5b}. In some embodiments, V is NR^{5a}R^{5b}, wherein R^{5a} and R^{5b}, together with the nitrogen atom to which each is attached, combine to form optionally substituted C₂-C₉ heterocyclyl.

In some embodiments, V is:

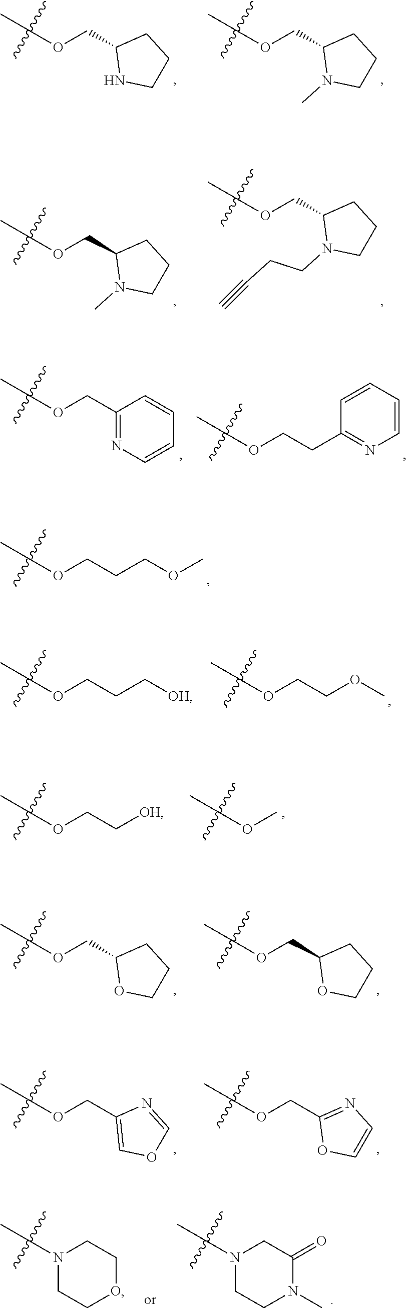

In some embodiments, V is:

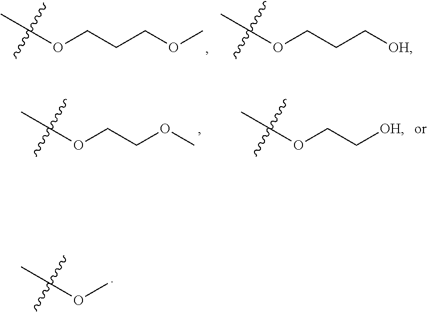

In some embodiments, V is:

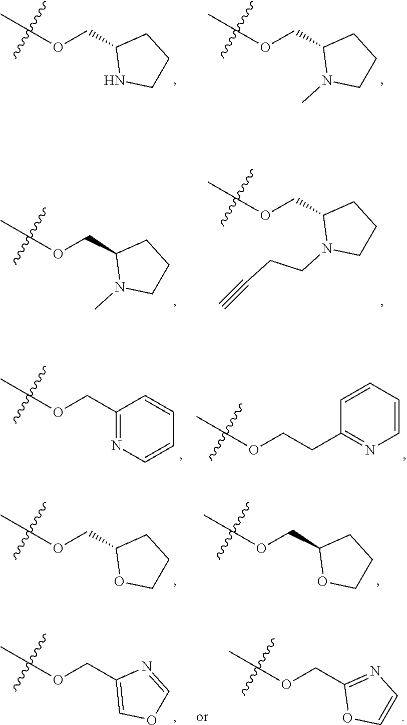

In some embodiments, V is:

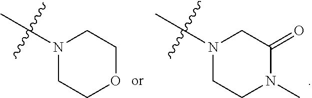

In some embodiments, the Ras binding moiety (e.g., K-Ras binding moiety) includes the structure:

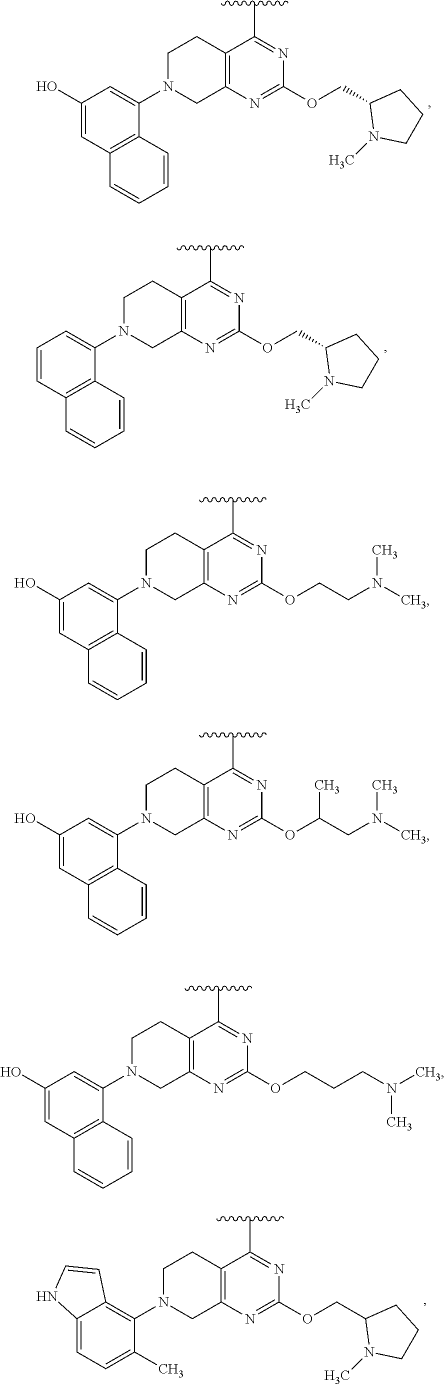 [SMILES: *c1nc(OC[C@@H]2CCCN2C)nc2c1CCN(c1cc(O)cc3ccccc13)C2 ; *c1nc(OC[C@@H]2CCCN2C)nc2c1CCN(c1cccc3ccccc13)C2 ; *c1nc(OCCN(C)C)nc2c1CCN(c1cc(O)cc3ccccc13)C2 ; *c1nc(OC(C)CN(C)C)nc2c1CCN(c1cc(O)cc3ccccc13)C2 ; *c1nc(OCCCN(C)C)nc2c1CCN(c1cc(O)cc3ccccc13)C2 ; *c1nc(OCC2CCCN2C)nc2c1CCN(c1c(C)ccc3[nH]ccc13)C2]

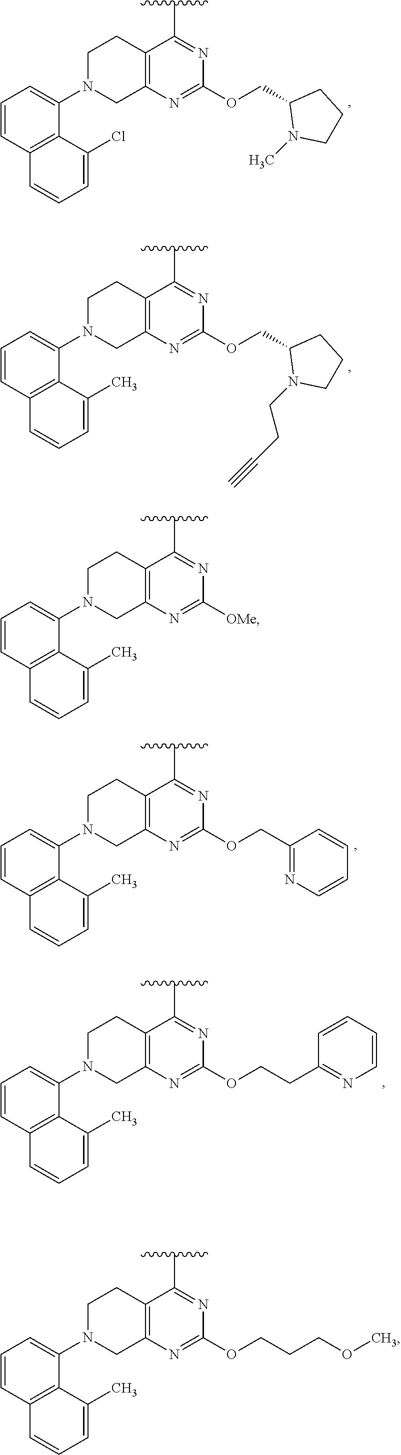 [SMILES: *c1nc(OC[C@@H]2CCCN2C)nc2c1CCN(c1cccc3cccc(Cl)c13)C2 ; *c1nc(OC[C@@H]2CCCN2CCC#C)nc2c1CCN(c1cccc3cccc(C)c13)C2 ; *c1nc(OC)nc2c1CCN(c1cccc3cccc(C)c13)C2 ; *c1nc(OCc2ccccn2)nc2c1CCN(c1cccc3cccc(C)c13)C2 ; *c1nc(OCCc2ccccn2)nc2c1CCN(c1cccc3cccc(C)c13)C2 ; *c1nc(OCCCOC)nc2c1CCN(c1cccc3cccc(C)c13)C2]

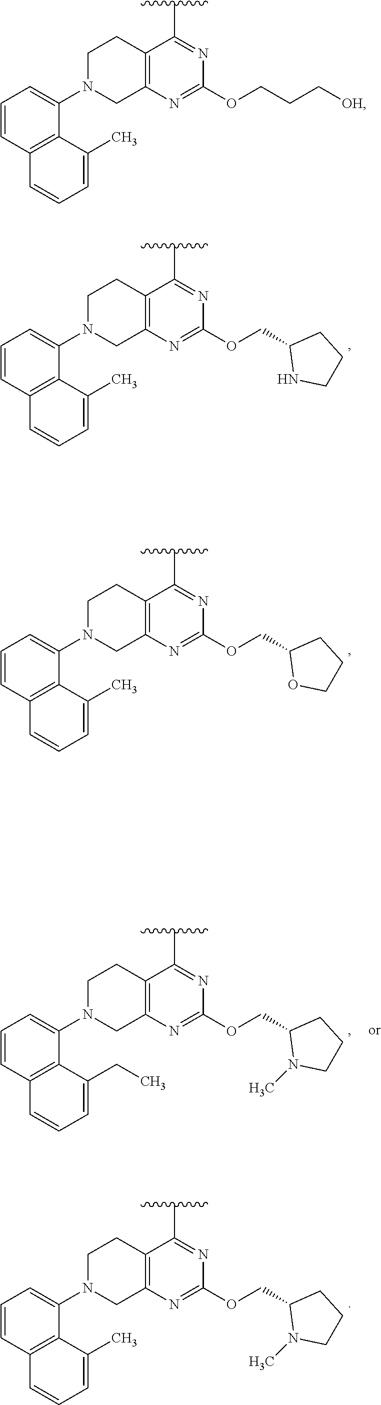 [SMILES: *c1nc(OCCCO)nc2c1CCN(c1cccc3cccc(C)c13)C2 ; *c1nc(OC[C@@H]2CCCN2)nc2c1CCN(c1cccc3cccc(C)c13)C2 ; *c1nc(OC[C@@H]2CCCO2)nc2c1CCN(c1cccc3cccc(C)c13)C2 ; *c1nc(OC[C@@H]2CCCN2C)nc2c1CCN(c1cccc3cccc(CC)c13)C2 ; *c1nc(OC[C@@H]2CCCN2C)nc2c1CCN(c1cccc3cccc(C)c13)C2]

In some embodiments, the Ras binding moiety (e.g., K-Ras binding moiety) includes the structure of Formula IV:

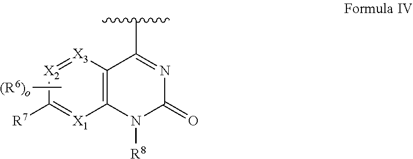

wherein o is 0, 1, or 2;

X¹, X² and X³ are each independently N, CH, or CR⁶;

each R⁶ is, independently, halo, CN, hydroxy, optionally substituted C₁-C₆ alkyl, or optionally substituted C₁-C₆ heteroalkyl, or

R⁶ is attached to the linker via a C₁-C₃ alkyl bridge or C₁-C₃ heteroalkyl bridge; and

R⁷ and R⁸ are, independently, optionally substituted C₆-C₁₀ aryl or optionally substituted C₂-C₉ heteroaryl,

or a pharmaceutically acceptable salt thereof.

In some embodiments, only one of X¹, X² and X³ is N.

In some embodiments, X₂ and X₃ are each CH or CR⁶, and X₁ is N.

In some embodiments, the Ras binding moiety (e.g., K-Ras binding moiety) includes the structure of Formula IVa:

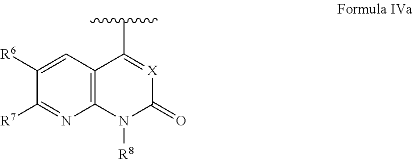

wherein R⁶ is hydrogen, halo, hydroxy, optionally substituted C₁-C₆ alkyl, or optionally substituted C₁-C₆ heteroalkyl; and

R⁷ and R⁸ are, independently, optionally substituted C₆-C₁₀ aryl or optionally substituted C₂-C₉ heteroaryl,

or a pharmaceutically acceptable salt thereof.

In some embodiments, Formula IV has the structure:

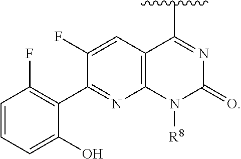

In some embodiments, the Ras binding moiety (e.g., K-Ras binding moiety) includes the structure of Formula IVb:

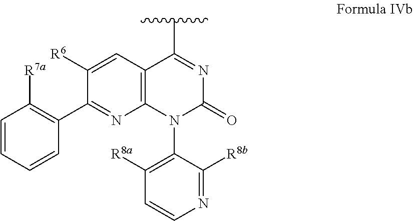

wherein R⁶, R^{7a}, R^{8a}, and R^{8b} are, independently, hydrogen, halo, hydroxy, optionally substituted C₁-C₆ alkyl, or optionally substituted C₁-C₆ heteroalkyl,

or a pharmaceutically acceptable salt thereof.

In some embodiments, R^{6a} is halo (e.g., fluoro). In some embodiments, R^{7a} is halo (e.g., fluoro). In some embodiments R^{8a} is optionally substituted C₁-C₆ alkyl (e.g., methyl). In some embodiments, R^{8b} is optionally substituted C₁-C₆ alkyl (e.g., iso-propyl).

In some embodiments, the Ras binding moiety (e.g., K-Ras binding moiety) includes the structure:

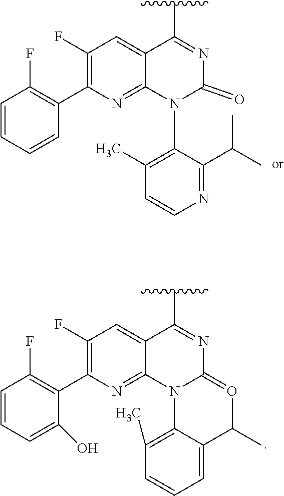

In some embodiments, the Ras binding moiety (e.g., K-Ras binding moiety) includes the structure of Formula V:

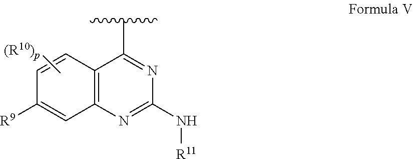

wherein p is 0, 1, 2, or 3;

R⁹ is optionally substituted C₆-C₁₀ aryl or optionally substituted C₂-C₉ heteroaryl;

each R¹⁰ is, independently, halo, CN, hydroxy, optionally substituted C₁-C₆ alkyl, or optionally substituted C₁-C₆ heteroalkyl, or R¹⁰ is attached to the linker via a C₁-C₃ alkylene or C₁-C₃ heteroalkylene bridge; and

R¹¹ is optionally substituted C₂-C₉ heteroaryl or optionally substituted C₂-C₉ heterocyclyl,

or a pharmaceutically acceptable salt thereof.

In some embodiments, the Ras binding moiety (e.g., K-Ras binding moiety) includes the structure:

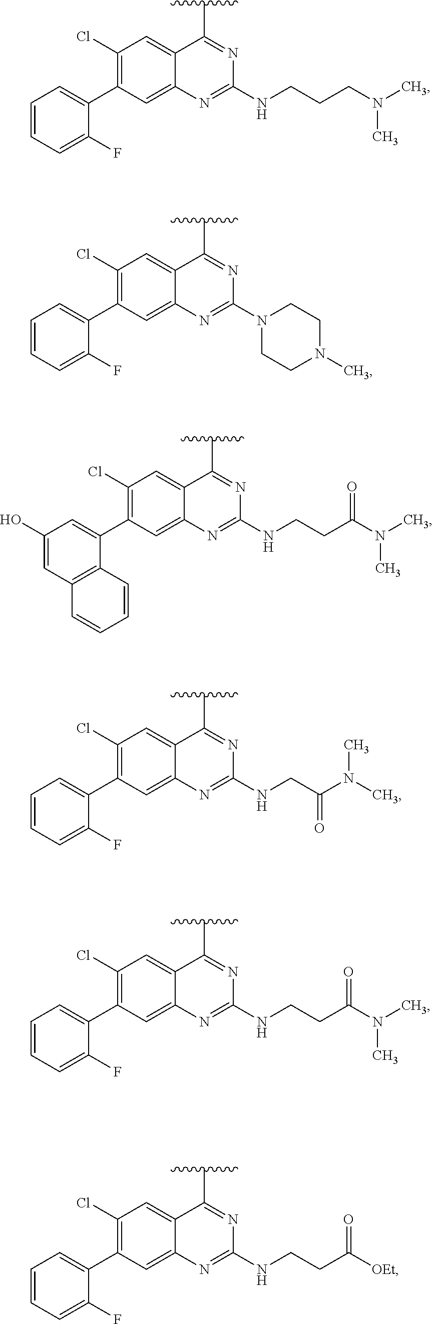 [SMILES: *c1nc(NCCCN(C)C)nc2cc(-c3ccccc3F)c(Cl)cc12 ; *c1nc(N2CCN(C)CC2)nc2cc(-c3ccccc3F)c(Cl)cc12 ; *c1nc(NCCC(=O)N(C)C)nc2cc(-c3cc(O)cc4ccccc34)c(Cl)cc12 ; *c1nc(NCC(=O)N(C)C)nc2cc(-c3ccccc3F)c(Cl)cc12 ; *c1nc(NCCC(=O)N(C)C)nc2cc(-c3ccccc3F)c(Cl)cc12 ; *c1nc(NCCC(=O)OCC)nc2cc(-c3ccccc3F)c(Cl)cc12]

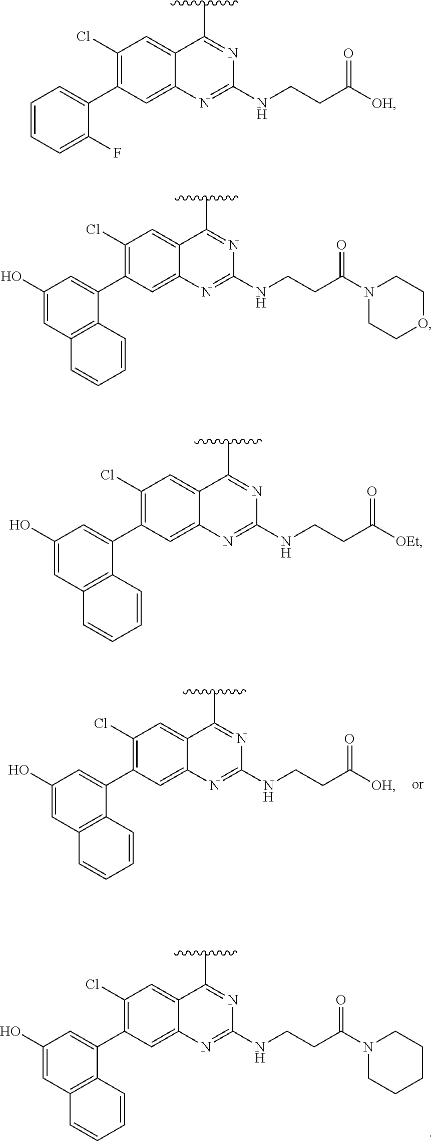 [SMILES: *c1nc(NCCC(=O)O)nc2cc(-c3ccccc3F)c(Cl)cc12 ; *c1nc(NCCC(=O)N2CCOCC2)nc2cc(-c3cc(O)cc4ccccc34)c(Cl)cc12 ; *c1nc(NCCC(=O)OCC)nc2cc(-c3cc(O)cc4ccccc34)c(Cl)cc12 ; *c1nc(NCCC(=O)O)nc2cc(-c3cc(O)cc4ccccc34)c(Cl)cc12 ; "or" ; *c1nc(NCCC(=O)N2CCCCC2)nc2cc(-c3cc(O)cc4ccccc34)c(Cl)cc12]

In some embodiments, the Ras binding moiety includes the structure of a Ras moiety described in WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223, the Ras binding moieties of which are herein incorporated by reference. In view of the disclosures herein as well as general knowledge, persons of skill in the art will understand how a cross-linking group of a compound of these references may be replaced with a selective cross-linking group of the present invention.

Compounds, or a pharmaceutically acceptable salt thereof, of the present invention include a linker between a Ras binding moiety (e.g., A, in Formula I) and a selective cross-linking group (e.g., B, in Formula I). As used herein, a “linker” refers to a divalent organic moiety connecting moiety A to moiety B in a compound of Formula I, such that the resulting compound is capable of achieving an IC50 of 2 μM or less in the Ras-RAF disruption assay protocol provided in Lim et al., Angew. Chem. Int. Ed. 53:199 (2014). In some embodiments, the linker positions a reactive atom of B about 5 to about 11 angstroms from the nearest atom of A. In some embodiments, the linker positions a reactive atom of B 4 to 9 atoms from the nearest atom of A. In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

The term “reactive”, when used in conjunction with a selective cross-linking group, refers to an electrophilic atom that reacts readily or at a practical rate under conventional conditions of organic synthesis or under physiological conditions to form a covalent bond with a nucleophilic functional group of a Ras protein, such as a carboxyl group, a hydroxy group, or a thiol group. This is in contrast to those atoms that either do not react or require strong catalysts or impractical reaction conditions in order to react (i.e., a “nonreactive” or “inert” group).

As used herein, “functional group” refers to an organic moiety within a Ras protein having the potential to make a covalent bond with a selective cross-linking group, as described herein. A functional group may be nucleophilic or electrophilic, as those terms are known in the art. Non-limiting examples of nucleophilic functional groups include carboxyl groups, hydroxy groups, and thiol groups. Non-limiting examples of Ras amino acids having a nucleophilic functional group include aspartic acid, glutamic acid, serine, threonine, tyrosine, cysteine, and lysine.

In some embodiments, a linker has the structure of Formula VI:

-A¹-(B¹)ₐ-(C)_{b}-(B²)_{c}-(D)-(B³)_{d}-(C²)ₑ-(B⁴)_{f}-A²- Formula VI

where A¹ is a bond between the linker and the Ras binding moiety; A² is a bond between the selective cross-linking group and the linker; B¹, B², B³, and B⁴ each, independently, is selected from optionally substituted C₁-C₂alkylene, optionally substituted C₁-C₃ heteroalkylene, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; a, b, c, d, e, and f are each, independently, 0 or 1; and D is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₂₋₆ heteroarylene, optionally substituted C₃₋₈ cycloalkylene, optionally substituted C₆₋₁₂ arylene, optionally substituted C₂-C₁₀ polyethylene glycol, or optionally substituted C₁₋₁₀ heteroalkylene, or a chemical bond linking A¹-(B¹)ₐ-(C¹)_{b}-(B²)_{c}- to -(B³)_{d}-(C²)ₑ-(B⁴)_{f}-A².

In some embodiments, the linker comprises a heteroaryl group, such as a phenyl group or a pyridyl group. Non-limiting examples of such linkers include:

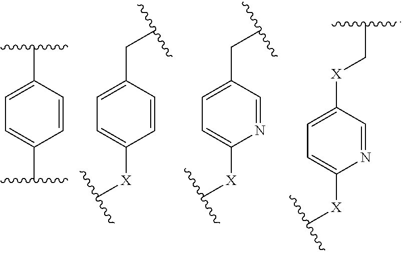

In some embodiments, the linker comprises a heterocyclyl group, such as a 3 to 8-membered heterocyclyl group. In some embodiments, the linker comprises a cycloalkyl group, such as a 3 to 8-membered carbocyclyl group.

In some embodiments, the linker is an optionally substituted heterocyclyl group, such as an optionally substituted 3 to 8-membered heterocyclyl group. In some embodiments, the linker is an optionally substituted cycloalkyl group, such as an optionally substituted 3 to 8-membered carbocyclyl group.

In some embodiments, the linker is as exemplified in any of Formulas VIIa to VIII. In these structures, when a nitrogen group is at position B, that nitrogen is part of the selective cross-linking group. When a carbon atom is at position B, that carbon atom is part of the linker.

In some embodiments, the compound A-L-B, or a pharmaceutically acceptable sat thereof, has the structure of any one of Formula VIIaₒ or VIIbₒ:

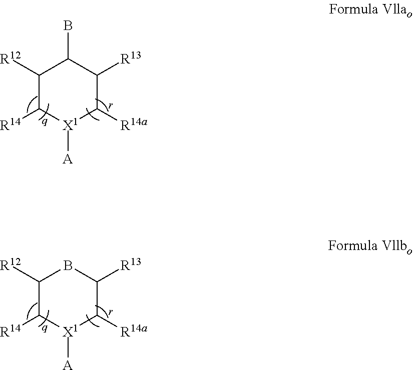

wherein q and r are, independently, 0, 1, or 2;

X¹ is N or CH; and

R¹², R¹³, R¹⁴ and R^{14a} are, independently, hydrogen, oxo, optionally substituted C₁-C₆ alkyl, optionally substituted C₁-C₆ heteroalkyl, or —CO₂-optionally substituted C₁-C₆ alkyl, wherein when R¹⁴ is not oxo, R¹⁴ optionally comprises a bond to A. In some embodiments, R¹², R¹³, R¹⁴ and R^{14a} are not simultaneously oxo. In some embodiments, only one of R¹², R¹³, R¹⁴ and R^{14a} is oxo.

In some embodiments, the compound A-L-B, or a pharmaceutically acceptable salt thereof, has the structure of any one of Formula VIIa or VIIb:

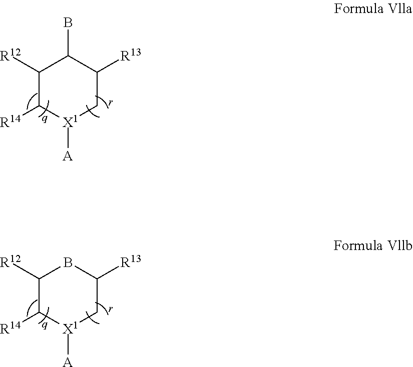

wherein q and r are, independently, 0, 1, or 2;

X¹ is N or CH;

R¹² and R¹³ are, independently, hydrogen, optionally substituted C₁-C₆ alkyl or optionally substituted C₁-C₆ heteroalkyl; and

R¹⁴ is hydrogen, optionally substituted C₁-C₆ alkyl, or optionally substituted C₁-C₆ heteroalkyl, wherein R¹⁴ optionally comprises a bond to A.

See also Formula VIIe, below, for a depiction of the linker moiety in these formulas.

In some embodiments, A-L-B, or a pharmaceutically acceptable salt thereof, is selected from the group consisting of:

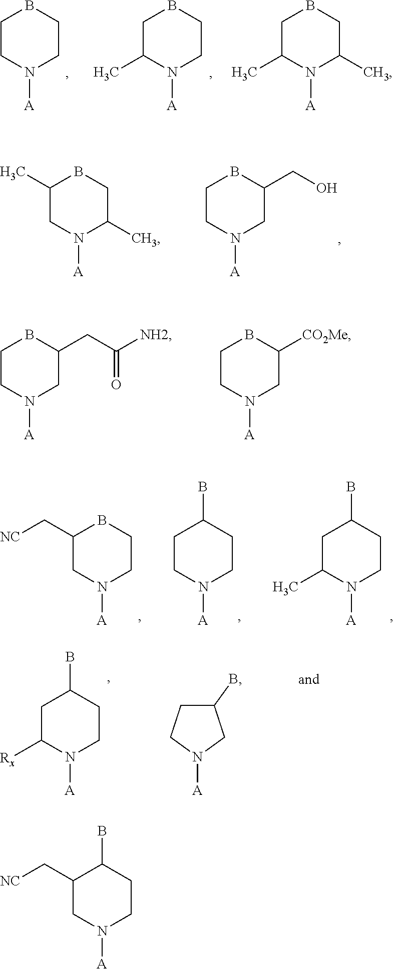

wherein Rₓ is an optionally substituted C₁-C₃ alkylene bridge or optionally substituted C₁-C₃ heteroalkylene bridge joined to A (see, e.g., WO 2018/206539).

In some embodiments, A-L-B, or a pharmaceutically acceptable salt thereof, is

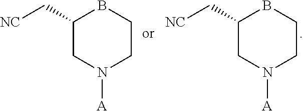

In some embodiments, -L-B is selected from the group consisting of:

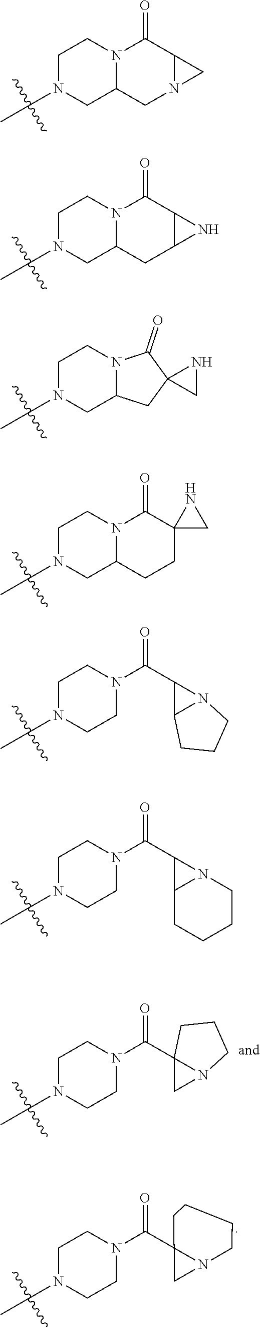

In some embodiments, A-L-B, or a pharmaceutically acceptable salt thereof, is the structure of Formula VIIc or Formula VIId:

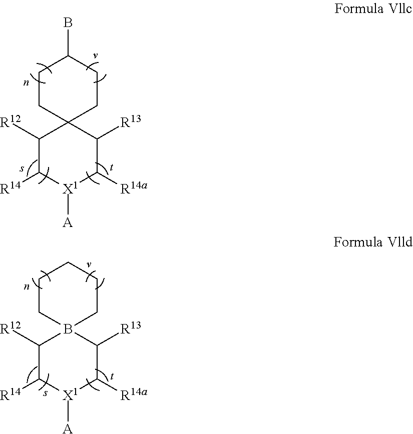

wherein s, t, u, and v are, independently, 0, 1, or 2;

X³ is N or CH; and

R¹⁵ and R¹⁶ are, independently, hydrogen, optionally substituted C₁-C₆ alkyl or optionally substituted C₁-C₆ heteroalkyl.

See also Formula VIIf, below, for a depiction of the linker moiety in these formulas.

In some embodiments, A-L-B, or a pharmaceutically acceptable salt thereof, is:

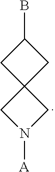

In some embodiments, the linker is acyclic. For example, the linker is the structure of Formula VIII:

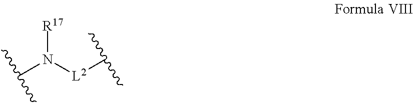

wherein R¹⁷ is hydrogen or optionally substituted C₁-C₆ alkyl; and

L² is optionally substituted C₁-C₄ alkylene or optionally substituted C₃-C₆ cycloalkyl.

In some embodiments, the linker is selected from the group consisting of:

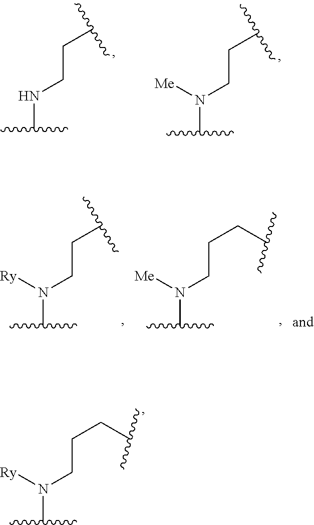

wherein R_{y} is an optionally substituted C₁-C₃ alkylene bridge or optionally substituted C₁-C₃ heteroalkylene bridge joined to A (see, e.g., WO 2018/206539).

In some embodiments, the linker has the structure:

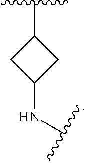 [SMILES: *NC1CC(*)C1]

Compounds, or a pharmaceutically acceptable salt thereof, of the present invention include a selective cross-linking group. As used herein, “selective cross-linking group” refers to a group which exhibits cross-linking reactivity preferentially with one or more Ras protein nucleophilic functional groups in comparison to other nucleophilic functional groups that exist in a Ras protein, under conventional conditions of organic synthesis or under physiological conditions. For example, in some embodiments, a selective cross-linking group reacts preferentially with a carboxyl group, a hydroxy group, or a thiol group, or a combination thereof, in comparison with other nucleophilic functional groups in a Ras protein. For example, in some embodiments, a selective cross-linking group reacts preferentially with a carboxyl group. In some embodiments, a selective cross-linking group reacts preferentially with a hydroxy group. In some embodiments, a selective cross-linking group reacts preferentially with a thiol group. In some embodiments, a selective cross-linking group reacts preferentially with a carboxyl group and a hydroxy group. In some embodiments, a selective cross-linking group reacts preferentially with a carboxyl group and a thiol group. In some embodiments, a selective cross-linking group reacts preferentially with a hydroxy group and a thiol group. Non-limiting examples of moieties which are “selective cross-linking groups” include, for example, a carbodiimide, an aminooxazoline, a chloroethyl urea, an aziridine, a trifluoromethyl ketone, a boronic acid, a boronic ester, N-ethoxycarbonyl-2-ethoxy-1,2-dihydroquinoline (EEDQ), an iso-EEDQ or other EEDQ derivative, an epoxide, an oxazolium, or a glycal. In some embodiments, a selective cross-linking group is a carbodiimide, an aminooxazoline, a chloroethyl urea, an aziridine, a trifluoromethyl ketone, a boronic acid, a boronic ester, an epoxide, or a glycal. In some embodiments, a selective cross-linking group is a carbodiimide, an aminooxazoline, a chloroethyl urea, or an aziridine.

In some embodiments, the selective cross-linking group is a C—O bond forming selective cross-linking group. In some embodiments, the selective cross-linking group is a C—S bond forming selective cross-linking group.

In some embodiments, the selective cross-linking group has the structure or is comprised within any one of Formula IX to XVIII.

In some embodiments, the selective cross-linking group is the structure of Formula IX:

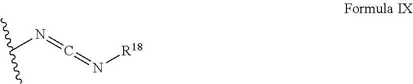

wherein R¹⁸ is optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl.

In some embodiments, the selective cross-linking group is selected from the group consisting of:

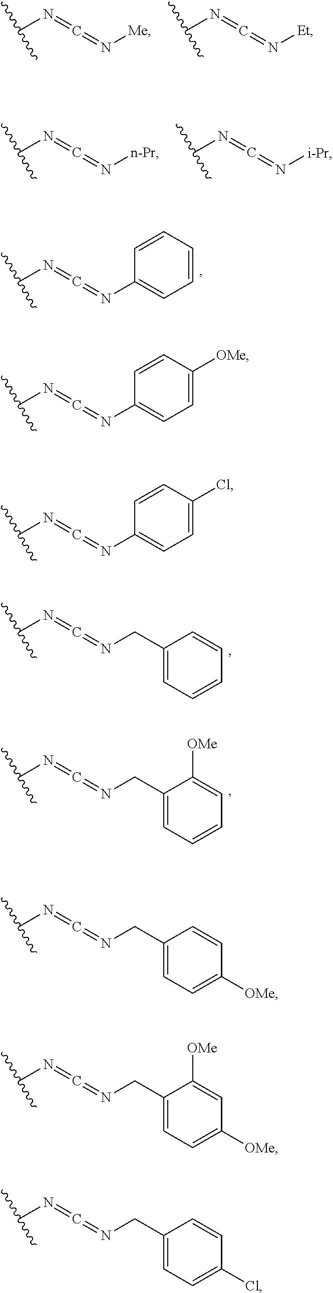

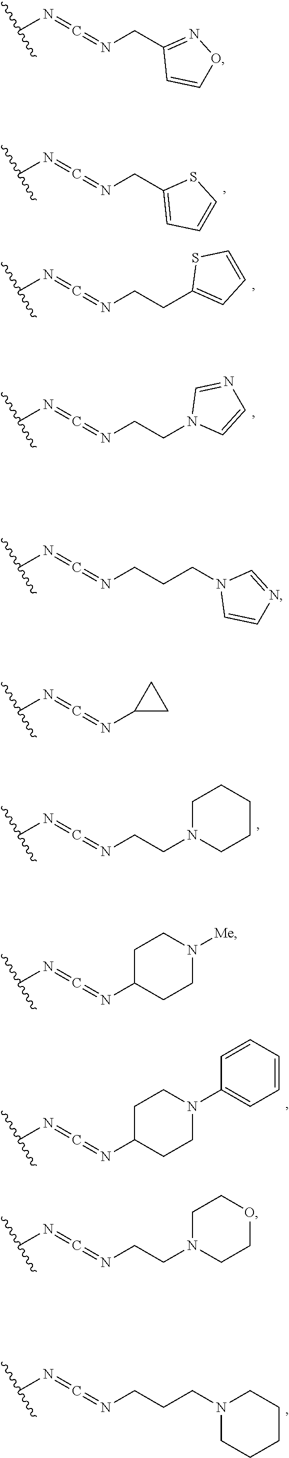

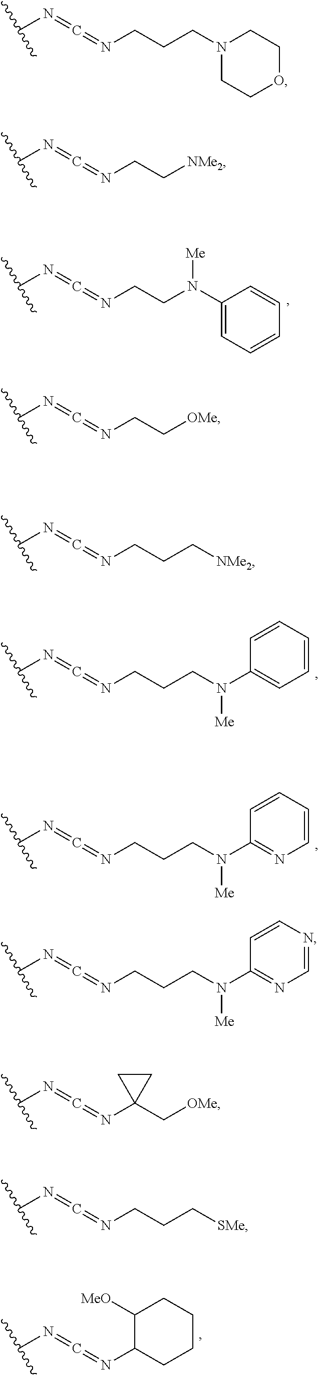

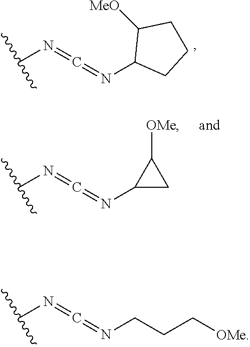 [SMILES: *N=C=NC1CCCC1OC ; *N=C=NC1CC1OC ; *N=C=NCCCOC]

In some embodiments, the selective cross-linking group is the structure of Formula Xa or Xb:

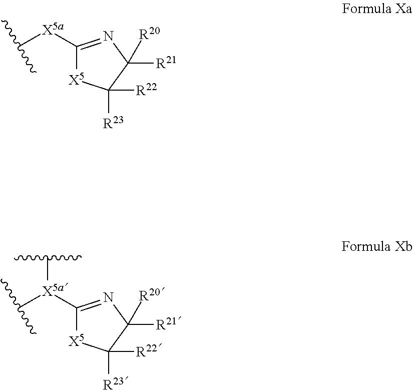

wherein X⁵ is O or S;

X^{5′} is O or S;

X^{5a} is absent or NR¹⁹;

X^{5a′} is N, wherein said N is a ring atom of an optionally substituted C₂-C₉ heterocyclyl group;

R¹⁹ is hydrogen, optionally substituted C₁-C₆ alkyl, optionally substituted C₆-C₁₀ aryl, optionally substituted C₂-C₉ heterocyclyl, or optionally substituted C₂-C₉ heteroaryl; and

R²⁰, R²¹, R²², R²³, R^{20′}, R^{21′}, R^{22′}, and R^{23′} are, independently, hydrogen or optionally substituted C₁-C₆ alkyl.

In some embodiments, the selective cross-linking group is selected from the group consisting of:

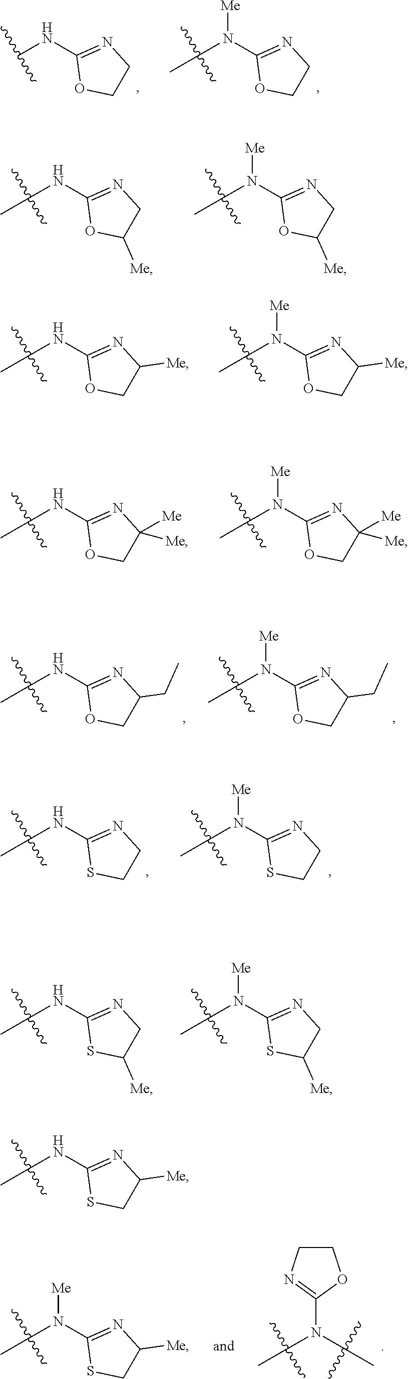

In some embodiments, the selective cross-linking group is the structure of Formula XIa or XIb:

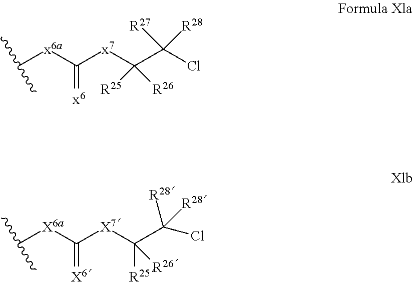

wherein X⁶ is O or S;

X^{6′} is O or S;

X^{6a} is absent or NR²⁴;

X^{6a′} is N, wherein said N is a ring atom of an optionally substituted C₂-C₉ heterocyclyl group;

X⁷ and X^{7′} are each O, S, or NR²⁹;

R²⁴ is hydrogen, optionally substituted C₁-C₆ alkyl, optionally substituted C₆-C₁₀ aryl, optionally substituted C₂-C₉ heterocyclyl, or optionally substituted C₂-C₉ heteroaryl; and

R²⁵, R²⁶, R²⁷, R²⁸, R²⁹, R^{25′}, R^{26′}, R^{27′}, and R^{28′} are, independently, hydrogen or optionally substituted C₁-C₆ alkyl.

In some embodiments, the selective cross-linking group is selected from the group consisting of:

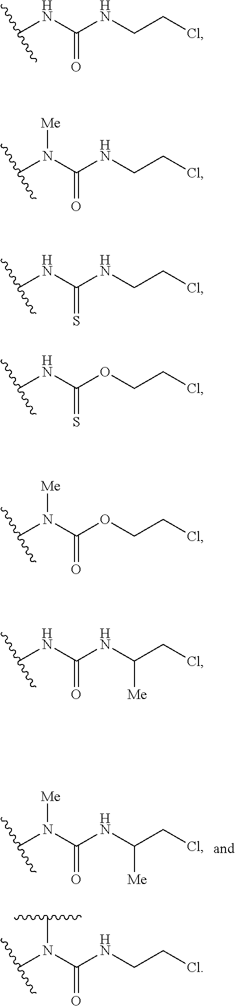

In some embodiments, the selective cross-linking group is the structure of Formula XIIa, XIIb, XIIc, XIId or XIIe:

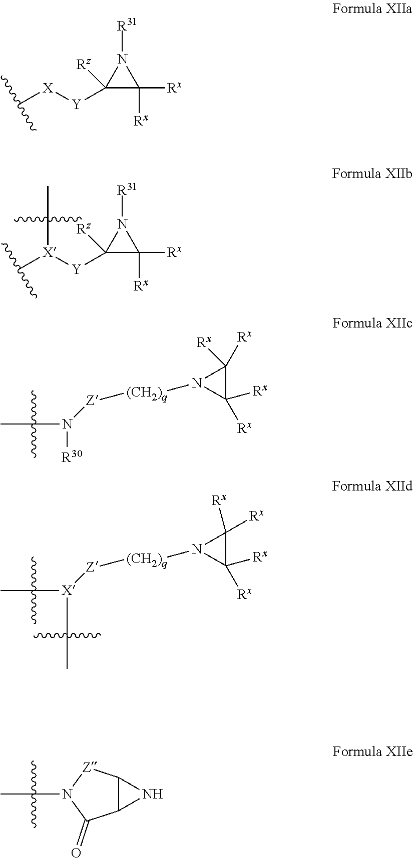

wherein X is absent or NR³⁰;

X′ is N, wherein said N is a ring atom of an optionally substituted C₂-C₉ heterocyclyl group;

Y is C(O), C(S) (that is, C═S), SO₂, or optionally substituted C₁-C₃ alkyl;

Z′ is C(O) or SO₂;

Z″ is —CH₂— or C(O);

q is 0, 1, or 2;

each R^{x} is, independently, hydrogen, CN, C(O)R^{y}, CO₂R^{y}, C(O)NR^{y}R^{y} optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl;

each R^{y} is, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl;

R^{z} is hydrogen or CH₃;

R³⁰ is hydrogen or optionally substituted C₁-C₆ alkyl;

R³¹ is hydrogen, —C(O)R³², —SO₂R³³, optionally substituted C₁-C₆ alkyl, optionally substituted C₁-C₆ heteroalkyl, optionally substituted C₆-C₁₀ aryl, optionally substituted C₂-C₉ heterocyclyl, or optionally substituted C₂-C₉ heteroaryl; and

R³² and R³³ are, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl.

In some embodiments, at least two of R³¹ and R^{x} is hydrogen. In some embodiments, R³¹ is CH₃, C(O)CH₃, SO₂CH₃, CH₂—C₆H₅, or CH₂CH₂OCH₃.

In some embodiments, the selective cross-linking group is selected from the group consisting of:

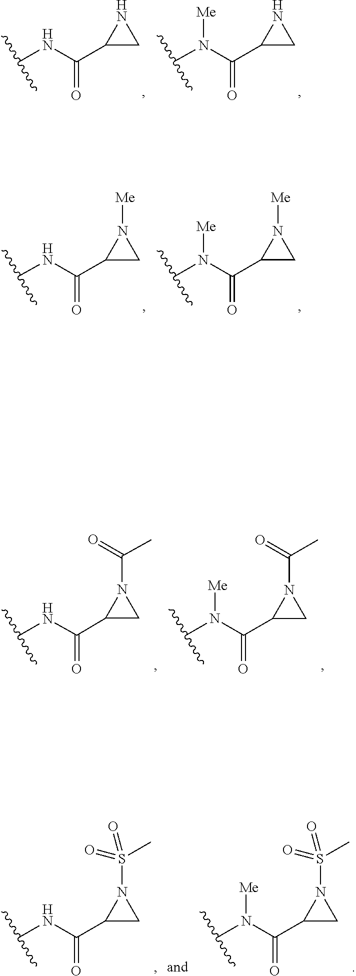

In some embodiments, the selective cross-linking group is selected from the group consisting of:

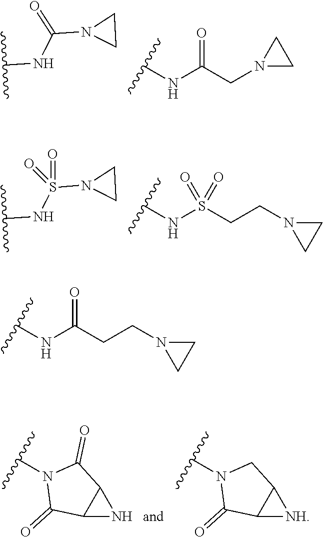

In some embodiments, the selective cross-linking group is selected from the groups consisting of:

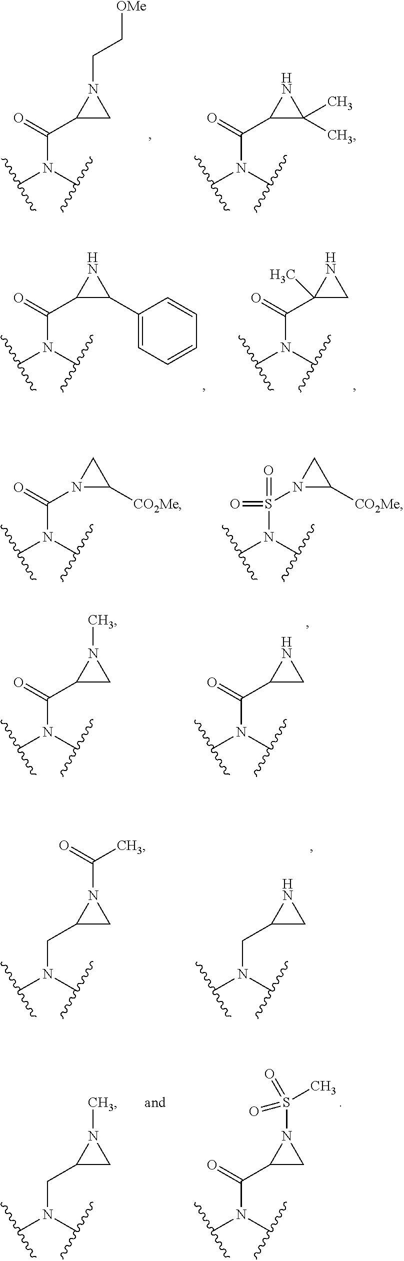

In some embodiments, the selective cross-linking group is selected from the groups consisting of:

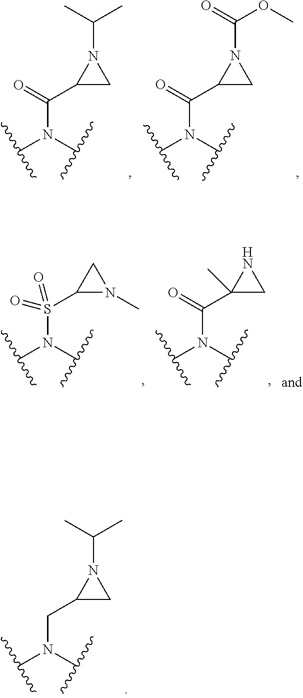

In some embodiments, a compound of the present invention has the structure:

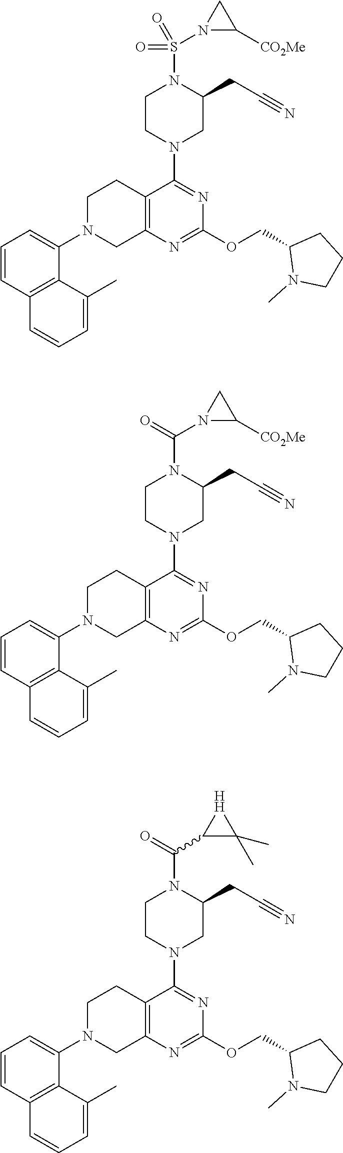

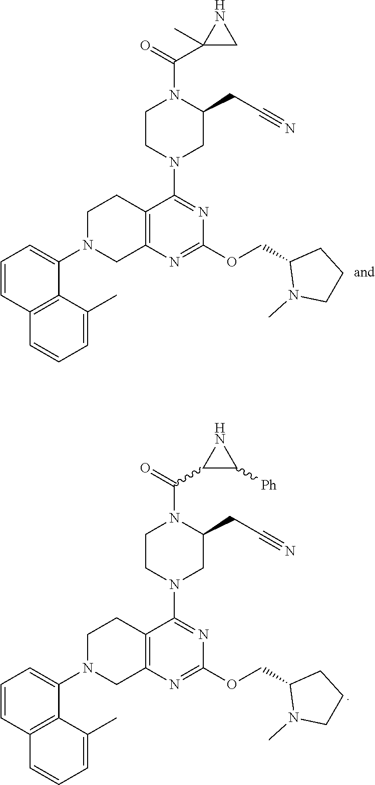 [SMILES: Cc1cccc2cccc(N3CCc4c(nc(OC[C@@H]5CCCN5C)nc4N4CCN(C(=O)C5(C)CN5)[C@@H](CC#N)C4)C3)c12 ; Cc1cccc2cccc(N3CCc4c(nc(OC[C@@H]5CCCN5C)nc4N4CCN(C(=O)C5NC5c5ccccc5)[C@@H](CC#N)C4)C3)c12]

In some embodiments, a compound of the present invention has the structure:

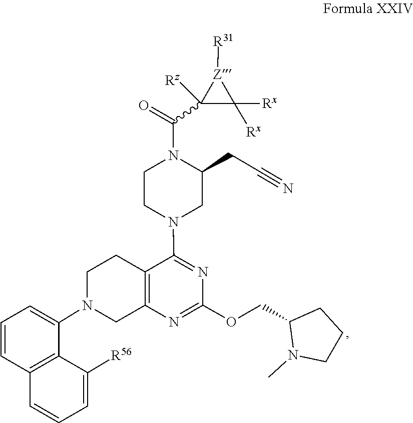

wherein R³¹ is absent, hydrogen, C(O)CH₃, SO₂CH₃, optionally substituted C₁-C₆ alkyl, optionally substituted C₁-C₆ heteroalkyl, optionally substituted C₆-C₁₀ aryl, optionally substituted C₁-C₃ alkyl-C₆-C₁₀ aryl, optionally substituted C₂-C₉ heterocyclyl, or optionally substituted C₁-C₃ alkyl-C₂-C₉ heterocyclyl;

R⁵⁶ is CH₃ or Cl;

R^{z} is hydrogen, optionally substituted C₁-C₃ alkyl;

each R^{x} is, independently, hydrogen, CO₂CH₃, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, optionally substituted C₂-C₆ alkenyl, or optionally substituted C₂-C₆ alkynyl; and

Z′″ is N or O.

In some embodiments, a compound of the present invention has the structure:

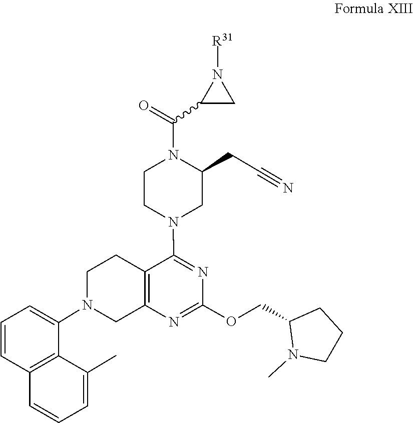

wherein R³¹ is hydrogen, CH₃, C(O)CH₃, SO₂CH₃, CH₂—C₆H₅, or CH₂CH₂OCH₃.

In some embodiments, a compound of the present invention has the structure:

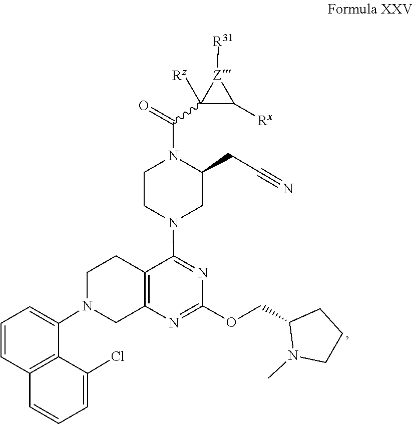

wherein R³¹ is absent, hydrogen, C(O)CH₃, optionally substituted C₁-C₆ alkyl, optionally substituted C₁-C₆ heteroalkyl, optionally substituted C₆-C₁₀ aryl, optionally substituted C₁-C₃ alkyl-C₆-C₁₀ aryl, optionally substituted C₂-C₉ heterocyclyl, or optionally substituted C₁-C₃ alkyl-C₂-C₉ heterocyclyl;

R^{z} is hydrogen, optionally substituted C₁-C₃ alkyl;

R^{x} is hydrogen, CO₂CH₃, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, optionally substituted C₂-C₆ alkenyl, or optionally substituted C₂-C₆ alkynyl; and

Z′″ is N or O.

In some embodiments, the selective cross-linking group is an optionally substituted aziridine. In some embodiments, the selective cross-linking group is an optionally substituted epoxide.

In some embodiments, the selective cross-linking group is:

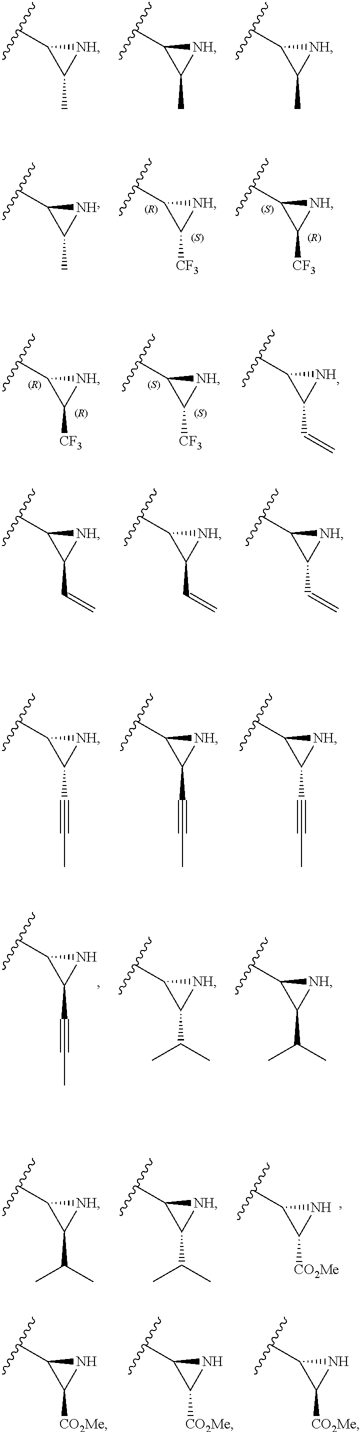

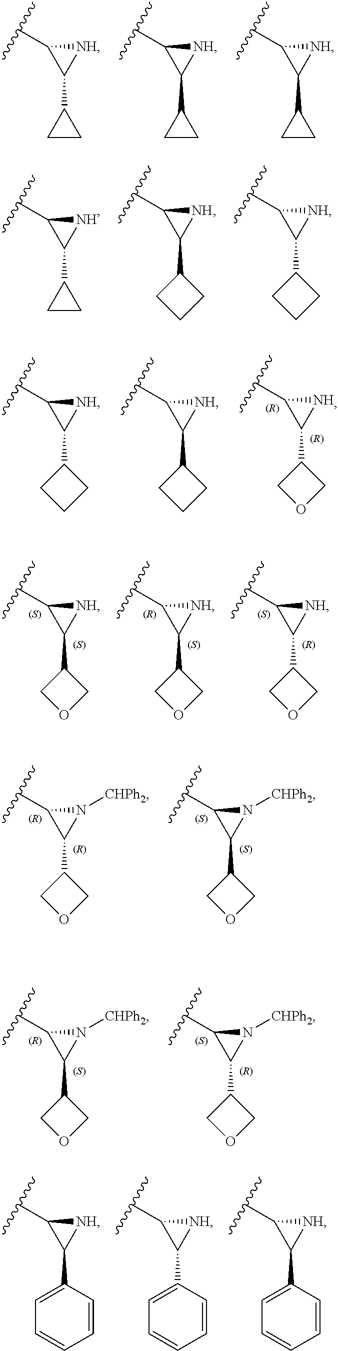

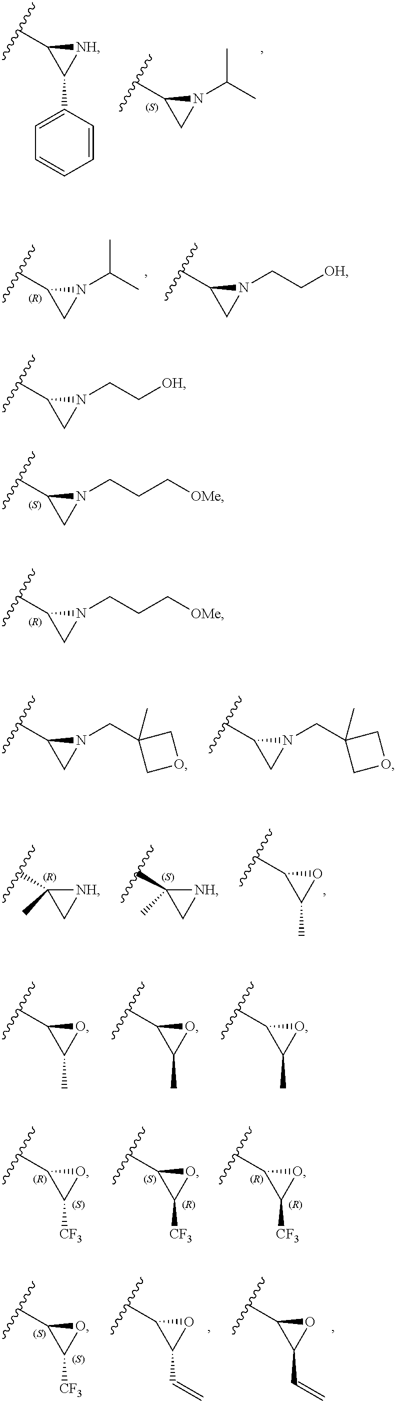

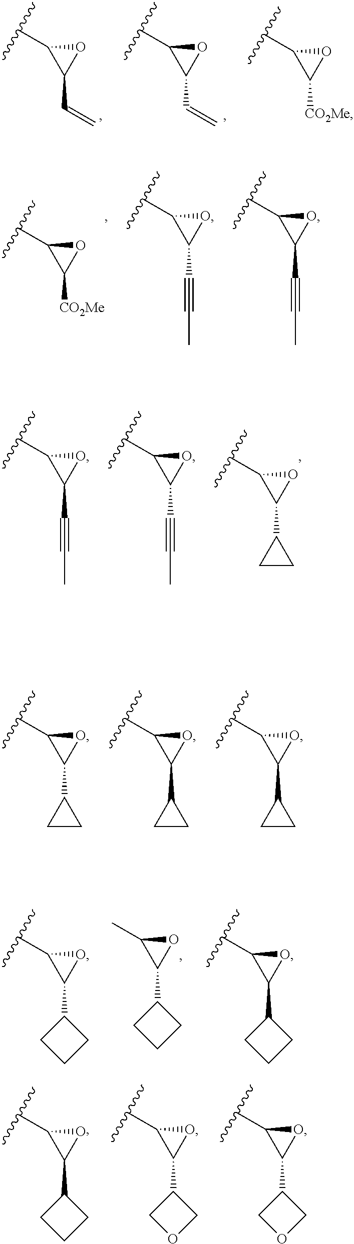

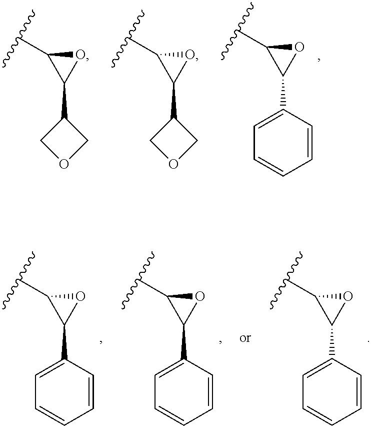 [SMILES: *[C@H]1O[C@H]1C1COC1 ; *[C@@H]1O[C@H]1C1COC1 ; *[C@H]1O[C@@H]1c1ccccc1 ; *[C@@H]1O[C@H]1c1ccccc1 ; *[C@H]1O[C@H]1c1ccccc1 ; *[C@@H]1O[C@@H]1c1ccccc1]

In some embodiments, the selective cross-linking group is:

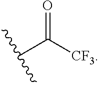 [SMILES: *C(=O)C(F)(F)F]

In some embodiments, the selective cross-linking is:

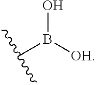 [SMILES: *B(O)O]

In some embodiments, the selective cross-linking group is the structure of Formula XIV:

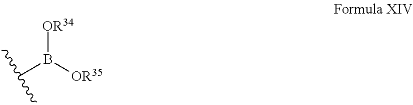

wherein R³⁴ and R³⁵ are, independently, optionally substituted C₁-C₆ alkyl, or R³⁴ and R³⁵ combine with the boron to which they are attached to form an optionally substituted heterocyclyl.

In some embodiments, the selective cross-linking group is the structure of Formula XV:

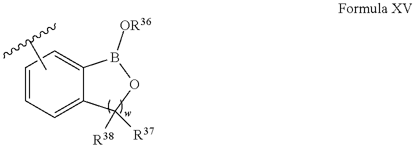

wherein w is 1 or 2;

R³⁶ is hydrogen or optionally substituted C₁-C₆ alkyl; and

each R³⁷ and R³⁸ is, independently, hydrogen or optionally substituted C₁-C₆ alkyl.

In some embodiments, the selective cross-linking group is selected from the group consisting of:

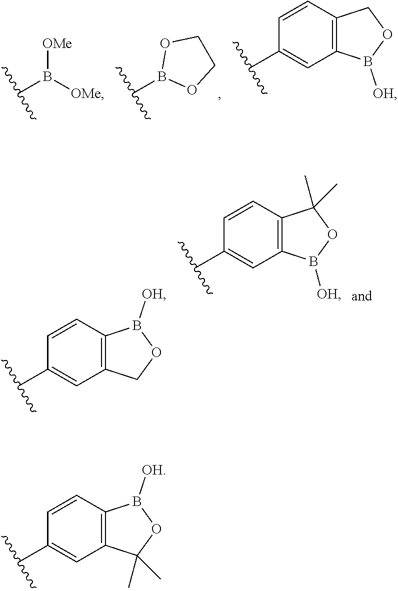

In some embodiments, the selective cross-linking group is the structure of Formula XVI:

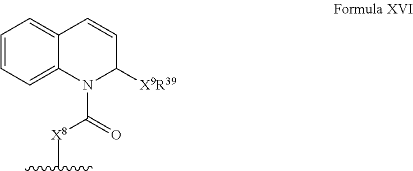

wherein X⁸ is absent, O, S, NR⁴⁰, or CH₂;

X⁹ is O, NR⁴¹, S, S(O), or S(O)₂;

R³⁹ is optionally substituted C₁-C₆ alkyl; and

R⁴⁰ and R⁴¹ are, independently, hydrogen or optionally substituted C₁-C₆ alkyl.

In some embodiments, the selective cross-linking group is:

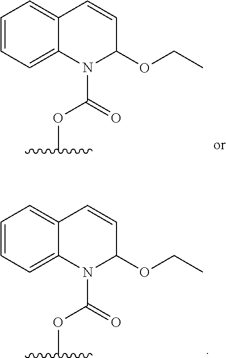 [SMILES: *OC(=O)N1c2ccccc2C=CC1OCC ; *OC(=O)N1c2ccccc2C=CC1OCC]

In some embodiments, the selective cross-linking group is the structure of Formula XVII:

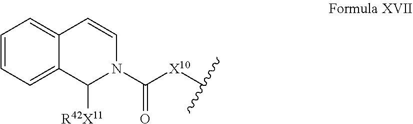

wherein X¹⁰ is absent, O, S, NR⁴³, or CH₂;

X¹¹ is O, NR⁴⁴, S, S(O), or S(O)₂;

R⁴² is optionally substituted C₁-C₆ alkyl; and

R⁴³ and R⁴⁴ are, independently, hydrogen or optionally substituted C₁-C₆ alkyl.

In some embodiments, the selective cross-linking group is:

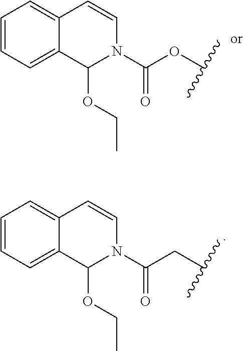 [SMILES: *OC(=O)N1C=Cc2ccccc2C1OCC ; *CC(=O)N1C=Cc2ccccc2C1OCC]

In some embodiments, the selective cross-linking group is the structure of Formula XVIII:

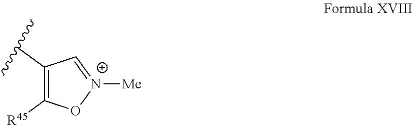

wherein R⁴⁵ is hydrogen or optionally substituted C₁-C₆ alkyl.

In some embodiments, the selective cross-linking group is:

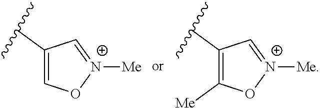 [SMILES: *c1co[n+](C)c1 ; *c1c[n+](C)oc1C]

In some embodiments, the selective cross-linking group is the structure of Formula XIX:

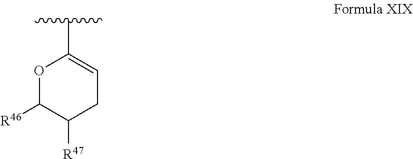

wherein R⁴⁶ and R⁴⁷ are, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl.

In some embodiments, a compound of the present invention has the structure of Formula XX or XXI:

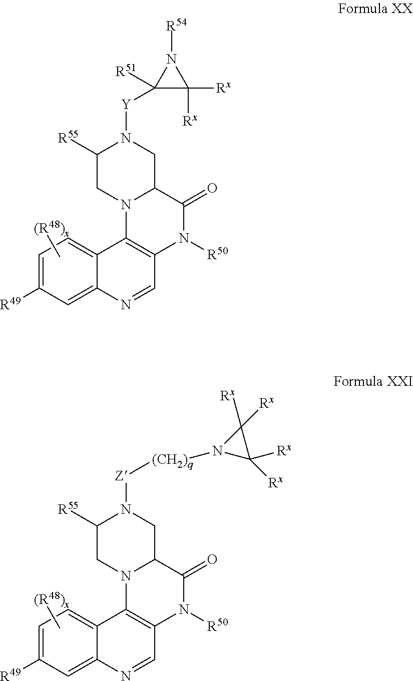

wherein Y is C(O), C(S), SO₂, or optionally substituted C₁-C₆ alkyl;

Z′ is C(O) or SO₂;

q is 0, 1 or 2;

x is 0, 1, 2 or 3;

each R^{X} is, independently, hydrogen, CN, C(O)R^{y}, CO₂R^{y}, C(O)NR^{y}R^{y} optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl;

each R^{y} is, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl;

each R⁴⁸ is, independently, CN, halo, hydroxy, optionally substituted C₁-C₆ alkyl, or optionally substituted C₁-C₆ heteroalkyl, or

R⁴⁹ is optionally substituted C₆-C₁₀ aryl or optionally substituted C₂-C₉ heteroaryl;

R⁵⁰ is hydrogen or C₁-C₆ alkyl;

R⁵¹ is hydrogen, CN or C₁-C₆ alkyl;

R⁵⁴ is hydrogen, —C(O)R³², —SO₂R³³, optionally substituted C₁-C₆ alkyl, optionally substituted C₁-C₆ heteroalkyl, optionally substituted C₆-C₁₀ aryl, optionally substituted C₂-C₉ heterocyclyl, or optionally substituted C₂-C₉ heteroaryl; and

R⁵⁵ is hydrogen or optionally substituted C₁-C₆ alkyl.

In some embodiments, R⁵¹, R⁵⁴ and R^{x} are each hydrogen.

In some embodiments, a compound of the present invention has the structure of Formula XXII or XXIII:

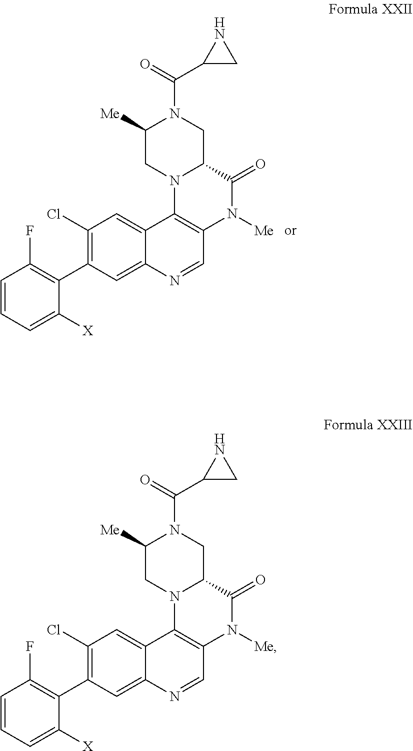

wherein X is hydrogen or hydroxy.

In some embodiments, a selective cross-linking group is an epoxide of the following formula:

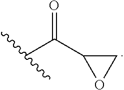 [SMILES: *C(=O)C1CO1]

In some embodiments, a compound of the present invention is selected from Table 1:

TABLE 1
Certain Compounds of the Present Invention

| # | Structure |
|---|---|
| 1 | 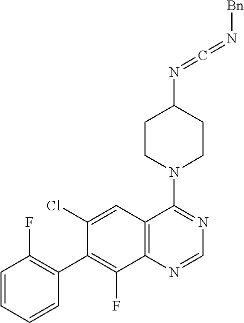 [SMILES: Fc1ccccc1-c1c(Cl)cc2c(N3CCC(N=C=NCc4ccccc4)CC3)ncnc2c1F] |
| 2 | 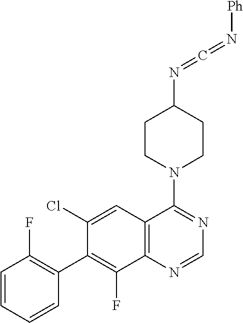 [SMILES: Fc1ccccc1-c1c(Cl)cc2c(N3CCC(N=C=Nc4ccccc4)CC3)ncnc2c1F] |
| 3 | 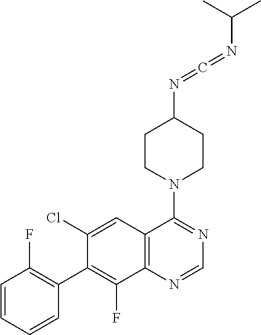 [SMILES: CC(C)N=C=NC1CCN(c2ncnc3c(F)c(-c4ccccc4F)c(Cl)cc23)CC1] |
| 4 | 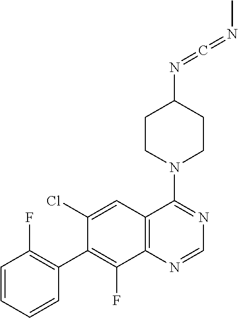 [SMILES: CN=C=NC1CCN(c2ncnc3c(F)c(-c4ccccc4F)c(Cl)cc23)CC1] |
| 5 | 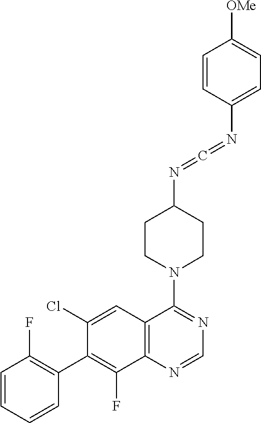 [SMILES: COc1ccc(N=C=NC2CCN(c3ncnc4c(F)c(-c5ccccc5F)c(Cl)cc34)CC2)cc1] |
| 6 | 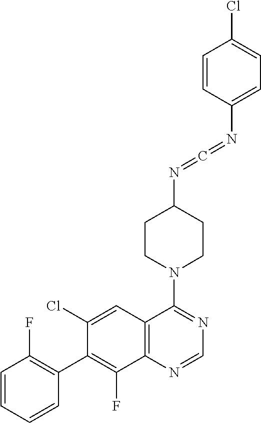 [SMILES: Fc1ccccc1-c1c(Cl)cc2c(N3CCC(N=C=Nc4ccc(Cl)cc4)CC3)ncnc2c1F] |
| 7 | 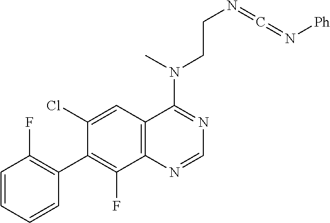 [SMILES: CN(CCN=C=Nc1ccccc1)c1ncnc2c(F)c(-c3ccccc3F)c(Cl)cc12] |
| 8 | 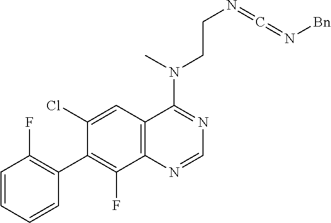 [SMILES: CN(CCN=C=NCc1ccccc1)c1ncnc2c(F)c(-c3ccccc3F)c(Cl)cc12] |
| 9 | 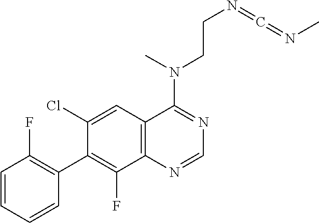 [SMILES: CN=C=NCCN(C)c1ncnc2c(F)c(-c3ccccc3F)c(Cl)cc12] |
| 10 | 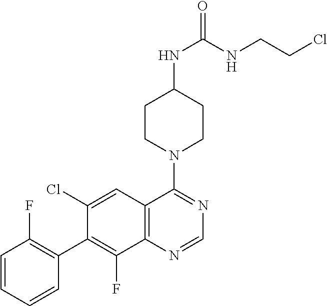 [SMILES: O=C(NCCCl)NC1CCN(c2ncnc3c(F)c(-c4ccccc4F)c(Cl)cc23)CC1] |
| 11 | 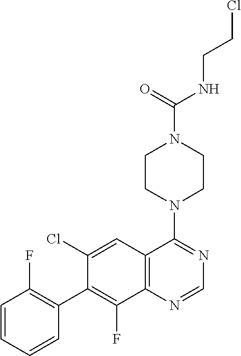 [SMILES: O=C(NCCCl)N1CCN(c2ncnc3c(F)c(-c4ccccc4F)c(Cl)cc23)CC1] |
| 12 | 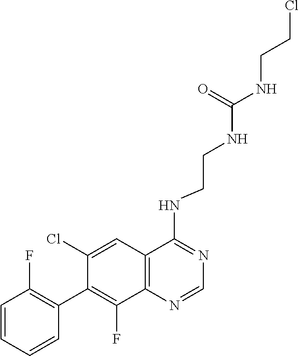 [SMILES: O=C(NCCCl)NCCNc1ncnc2c(F)c(-c3ccccc3F)c(Cl)cc12] |
| 13 | 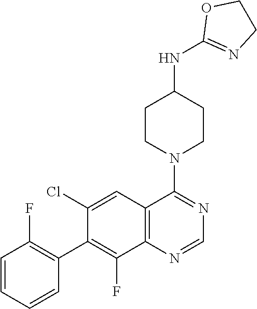 [SMILES: Fc1ccccc1-c1c(Cl)cc2c(N3CCC(NC4=NCCO4)CC3)ncnc2c1F] |
| 14 | 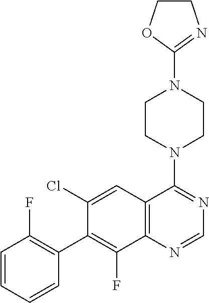 [SMILES: Fc1ccccc1-c1c(Cl)cc2c(N3CCN(C4=NCCO4)CC3)ncnc2c1F] |
| 15 | 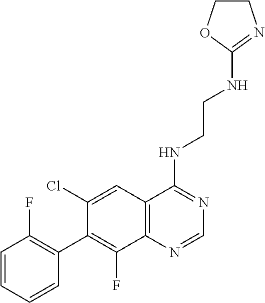 [SMILES: Fc1ccccc1-c1c(Cl)cc2c(NCCNC3=NCCO3)ncnc2c1F] |
| 16 | 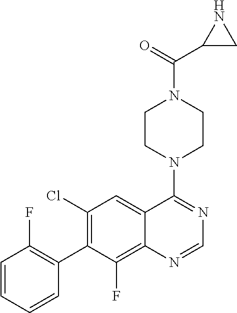 [SMILES: O=C(C1CN1)N1CCN(c2ncnc3c(F)c(-c4ccccc4F)c(Cl)cc23)CC1] |
| 17 | 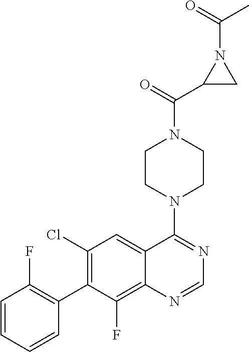 [SMILES: CC(=O)N1CC1C(=O)N1CCN(c2ncnc3c(F)c(-c4ccccc4F)c(Cl)cc23)CC1] |
| 18 | 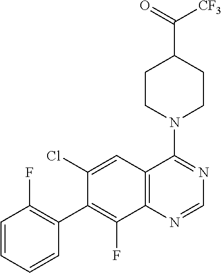 [SMILES: O=C(C1CCN(c2ncnc3c(F)c(-c4ccccc4F)c(Cl)cc23)CC1)C(F)(F)F] |
| 19 | 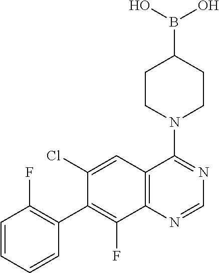 [SMILES: OB(O)C1CCN(c2ncnc3c(F)c(-c4ccccc4F)c(Cl)cc23)CC1] |
| 20 | 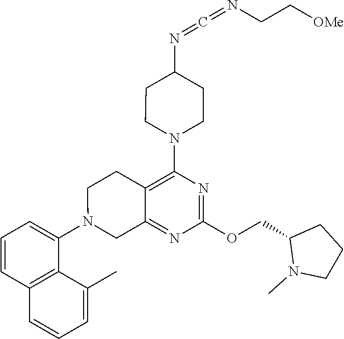 [SMILES: COCCN=C=NC1CCN(c2nc(OC[C@@H]3CCCN3C)nc3c2CCN(c2cccc4cccc(C)c24)C3)CC1] |
| 21 | 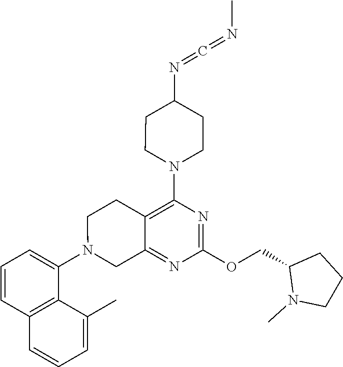 [SMILES: CN=C=NC1CCN(c2nc(OC[C@@H]3CCCN3C)nc3c2CCN(c2cccc4cccc(C)c24)C3)CC1] |
| 22 | 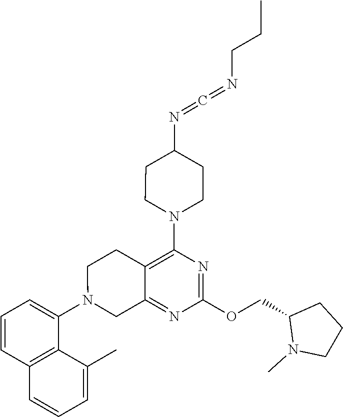 [SMILES: CCCN=C=NC1CCN(c2nc(OC[C@@H]3CCCN3C)nc3c2CCN(c2cccc4cccc(C)c24)C3)CC1] |
| 23 | 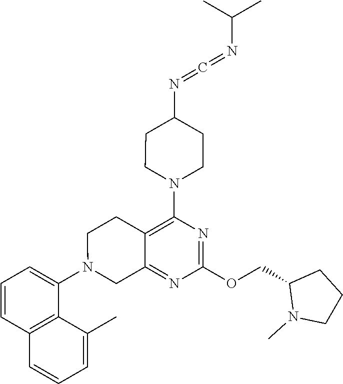 [SMILES: Cc1cccc2cccc(N3CCc4c(nc(OC[C@@H]5CCCN5C)nc4N4CCC(N=C=NC(C)C)CC4)C3)c12] |
| 24 | 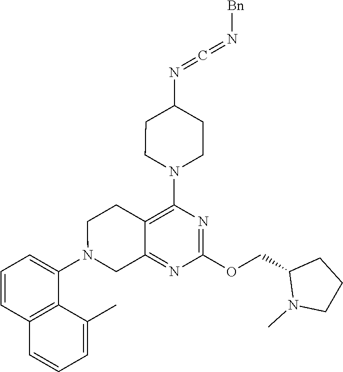 [SMILES: Cc1cccc2cccc(N3CCc4c(nc(OC[C@@H]5CCCN5C)nc4N4CCC(N=C=NCc5ccccc5)CC4)C3)c12] |
| 25 | 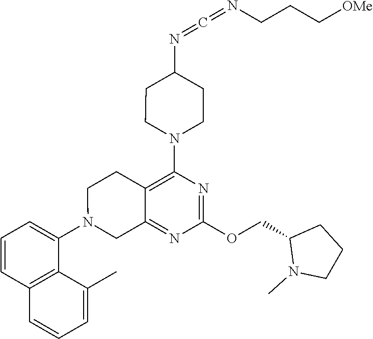 [SMILES: COCCCN=C=NC1CCN(c2nc(OC[C@@H]3CCCN3C)nc3c2CCN(c2cccc4cccc(C)c24)C3)CC1] |
| 26 | 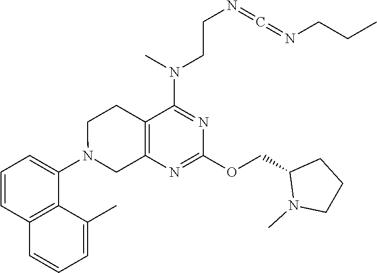 [SMILES: CCCN=C=NCCN(C)c1nc(OC[C@@H]2CCCN2C)nc2c1CCN(c1cccc3cccc(C)c13)C2] |
| 27 | 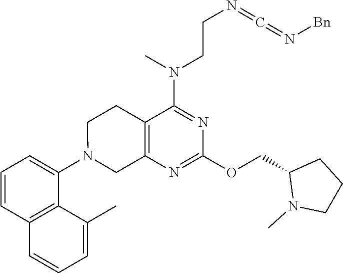 [SMILES: Cc1cccc2cccc(N3CCc4c(nc(OC[C@@H]5CCCN5C)nc4N(C)CCN=C=NCc4ccccc4)C3)c12] |
| 28 | 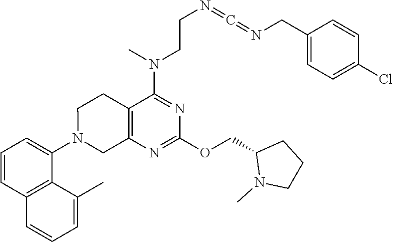 [SMILES: Cc1cccc2cccc(N3CCc4c(nc(OC[C@@H]5CCCN5C)nc4N(C)CCN=C=NCc4ccc(Cl)cc4)C3)c12] |
| 29 | 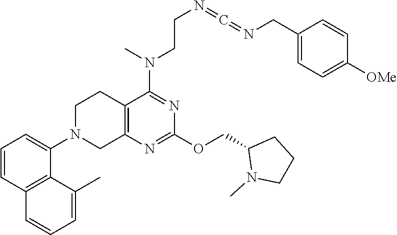 [SMILES: COc1ccc(CN=C=NCCN(C)c2nc(OC[C@@H]3CCCN3C)nc3c2CCN(c2cccc4cccc(C)c24)C3)cc1] |
| 30 | 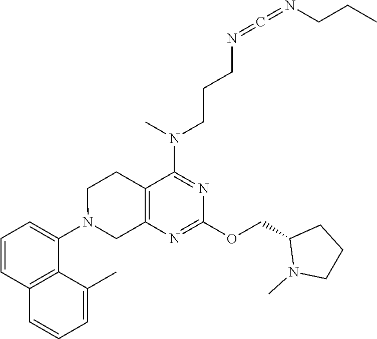 [SMILES: CCCN=C=NCCCN(C)c1nc(OC[C@@H]2CCCN2C)nc2c1CCN(c1cccc3cccc(C)c13)C2] |
| 31 | 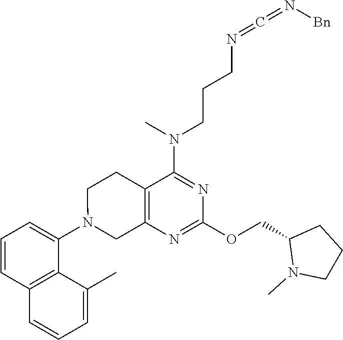 [SMILES: Cc1cccc2cccc(N3CCc4c(nc(OC[C@@H]5CCCN5C)nc4N(C)CCCN=C=NCc4ccccc4)C3)c12] |
| 32 | 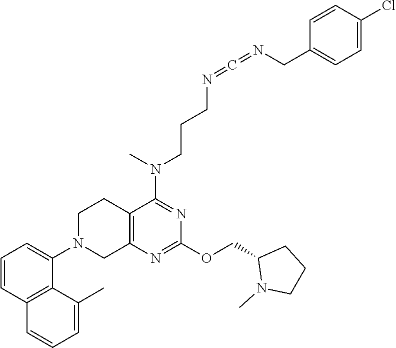 [SMILES: Cc1cccc2cccc(N3CCc4c(nc(OC[C@@H]5CCCN5C)nc4N(C)CCCN=C=NCc4ccc(Cl)cc4)C3)c12] |
| 33 | 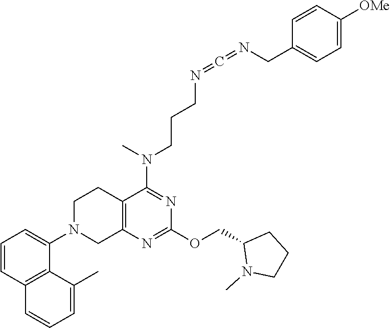 [SMILES: COc1ccc(CN=C=NCCCN(C)c2nc(OC[C@@H]3CCCN3C)nc3c2CCN(c2cccc4cccc(C)c24)C3)cc1] |
| 34 | 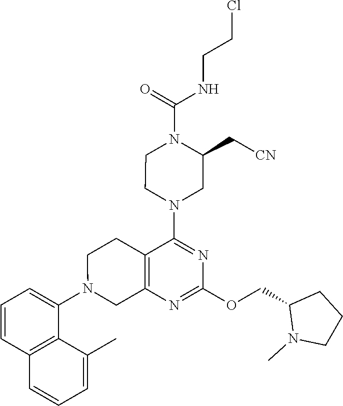 [SMILES: Cc1cccc2cccc(N3CCc4c(nc(OC[C@@H]5CCCN5C)nc4N4CCN(C(=O)NCCCl)[C@@H](CC#N)C4)C3)c12] |
| 35 | 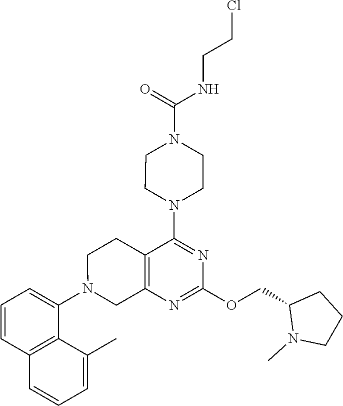 [SMILES: Cc1cccc2cccc(N3CCc4c(nc(OC[C@@H]5CCCN5C)nc4N4CCN(C(=O)NCCCl)CC4)C3)c12] |
| 36 | 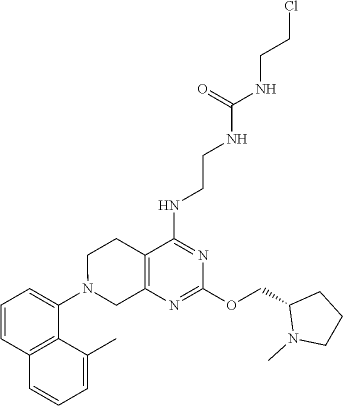 [SMILES: Cc1cccc2cccc(N3CCc4c(nc(OC[C@@H]5CCCN5C)nc4NCCNC(=O)NCCCl)C3)c12] |
| 37 | 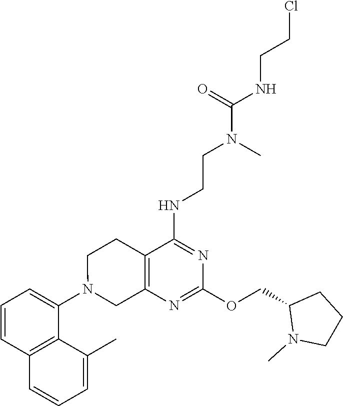 [SMILES: Cc1cccc2cccc(N3CCc4c(nc(OC[C@@H]5CCCN5C)nc4NCCN(C)C(=O)NCCCl)C3)c12] |
| 38 | 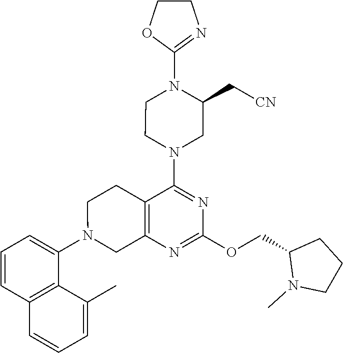 [SMILES: Cc1cccc2cccc(N3CCc4c(nc(OC[C@@H]5CCCN5C)nc4N4CCN(C5=NCCO5)[C@@H](CC#N)C4)C3)c12] |
| 39 | 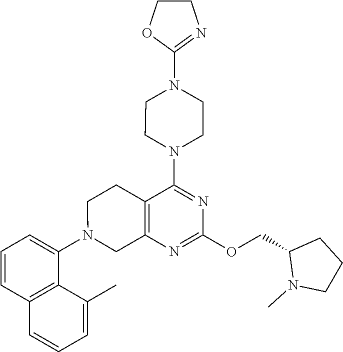 [SMILES: Cc1cccc2cccc(N3CCc4c(nc(OC[C@@H]5CCCN5C)nc4N4CCN(C5=NCCO5)CC4)C3)c12] |
| 40 | 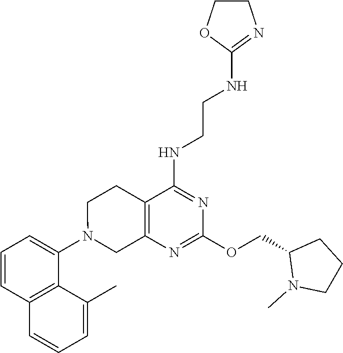 [SMILES: Cc1cccc2cccc(N3CCc4c(nc(OC[C@@H]5CCCN5C)nc4NCCNC4=NCCO4)C3)c12] |
| 41 | 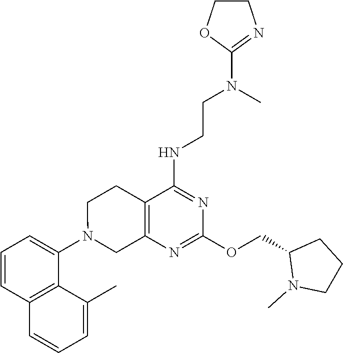 [SMILES: Cc1cccc2cccc(N3CCc4c(nc(OC[C@@H]5CCCN5C)nc4NCCN(C)C4=NCCO4)C3)c12] |
| 42 | 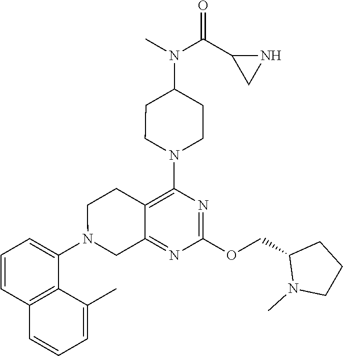 [SMILES: Cc1cccc2cccc(N3CCc4c(nc(OC[C@@H]5CCCN5C)nc4N4CCC(N(C)C(=O)C5CN5)CC4)C3)c12] |
| 43 | 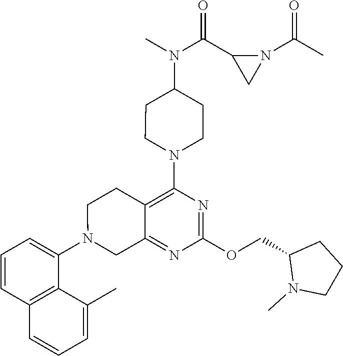 [SMILES: CC(=O)N1CC1C(=O)N(C)C1CCN(c2nc(OC[C@@H]3CCCN3C)nc3c2CCN(c2cccc4cccc(C)c24)C3)CC1] |
| 44 | 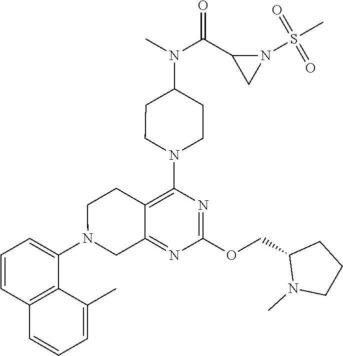 [SMILES: Cc1cccc2cccc(N3CCc4c(nc(OC[C@@H]5CCCN5C)nc4N4CCC(N(C)C(=O)C5CN5S(C)(=O)=O)CC4)C3)c12] |
| 45 | 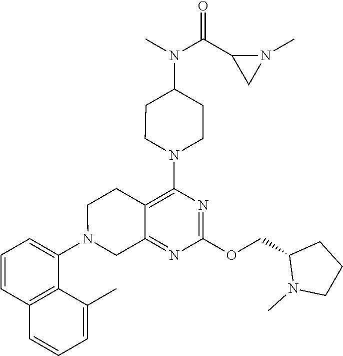 [SMILES: Cc1cccc2cccc(N3CCc4c(nc(OC[C@@H]5CCCN5C)nc4N4CCC(N(C)C(=O)C5CN5C)CC4)C3)c12] |
| 46 | 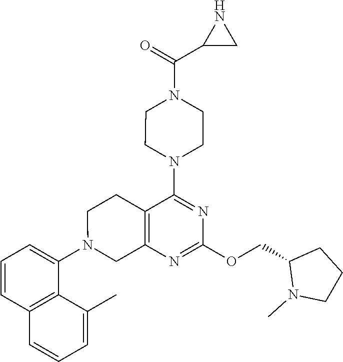 [SMILES: Cc1cccc2cccc(N3CCc4c(nc(OC[C@@H]5CCCN5C)nc4N4CCN(C(=O)C5CN5)CC4)C3)c12] |
| 47 | 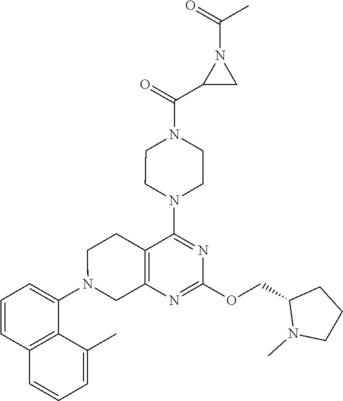 [SMILES: CC(=O)N1CC1C(=O)N1CCN(c2nc(OC[C@@H]3CCCN3C)nc3c2CCN(c2cccc4cccc(C)c24)C3)CC1] |
| 48 | 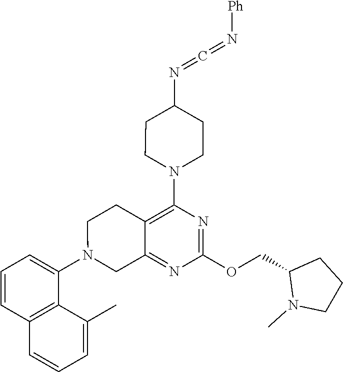 [SMILES: Cc1cccc2cccc(N3CCc4c(nc(OC[C@@H]5CCCN5C)nc4N4CCC(N=C=Nc5ccccc5)CC4)C3)c12] |
| 49 | 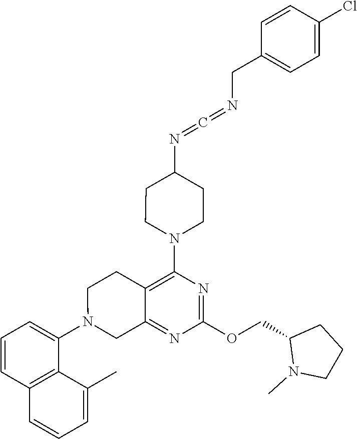 [SMILES: Cc1cccc2cccc(N3CCc4c(nc(OC[C@@H]5CCCN5C)nc4N4CCC(N=C=NCc5ccc(Cl)cc5)CC4)C3)c12] |
| 50 | 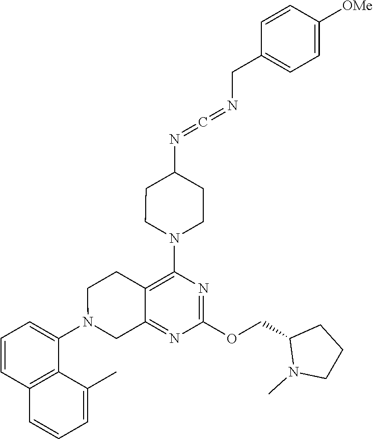 [SMILES: COc1ccc(CN=C=NC2CCN(c3nc(OC[C@@H]4CCCN4C)nc4c3CCN(c3cccc5cccc(C)c35)C4)CC2)cc1] |
| 51 | 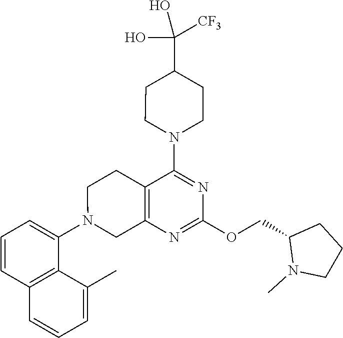 [SMILES: Cc1cccc2cccc(N3CCc4c(nc(OC[C@@H]5CCCN5C)nc4N4CCC(C(O)(O)C(F)(F)F)CC4)C3)c12] |
| 52 | 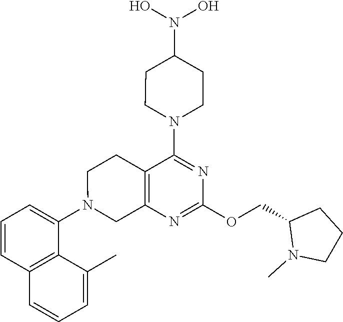 [SMILES: Cc1cccc2cccc(N3CCc4c(nc(OC[C@@H]5CCCN5C)nc4N4CCC(N(O)O)CC4)C3)c12] |
| 53 | 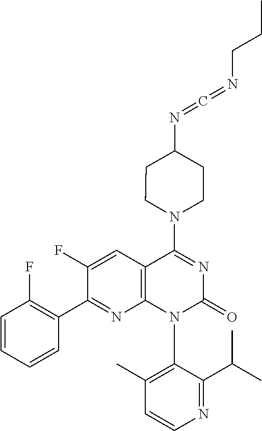 [SMILES: CCCN=C=NC1CCN(c2nc(=O)n(-c3c(C)ccnc3C(C)C)c3nc(-c4ccccc4F)c(F)cc23)CC1] |
| 54 | 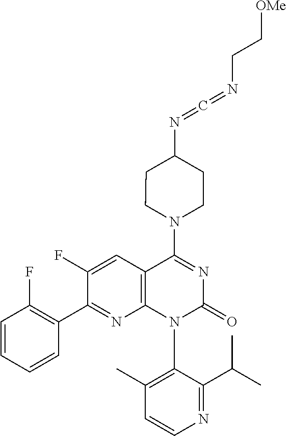 [SMILES: COCCN=C=NC1CCN(c2nc(=O)n(-c3c(C)ccnc3C(C)C)c3nc(-c4ccccc4F)c(F)cc23)CC1] |
| 55 | 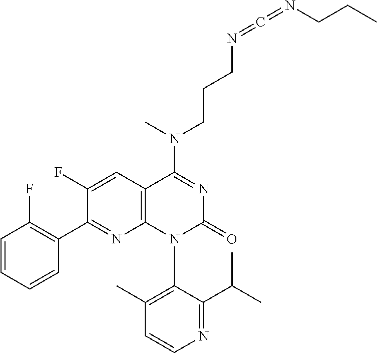 [SMILES: CCCN=C=NCCCN(C)c1nc(=O)n(-c2c(C)ccnc2C(C)C)c2nc(-c3ccccc3F)c(F)cc12] |
| 56 | 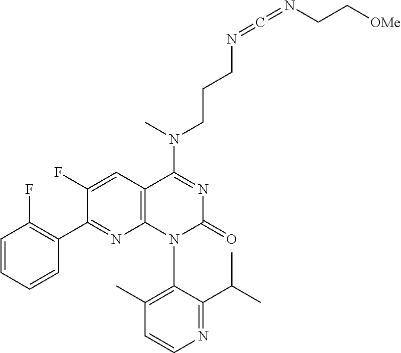 [SMILES: COCCN=C=NCCCN(C)c1nc(=O)n(-c2c(C)ccnc2C(C)C)c2nc(-c3ccccc3F)c(F)cc12] |
| 57 | 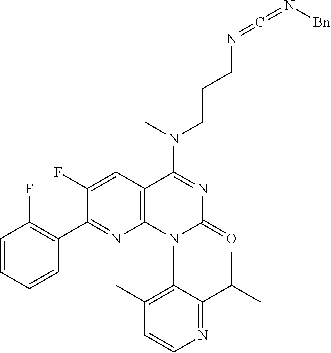 [SMILES: Cc1ccnc(C(C)C)c1-n1c(=O)nc(N(C)CCCN=C=NCc2ccccc2)c2cc(F)c(-c3ccccc3F)nc21] |
| 58 | 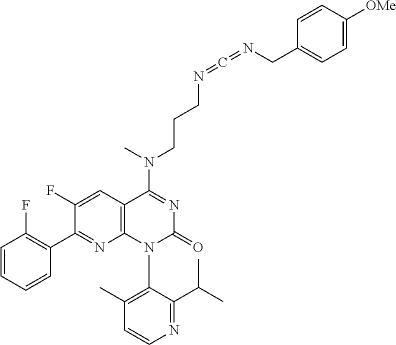 [SMILES: COc1ccc(CN=C=NCCCN(C)c2nc(=O)n(-c3c(C)ccnc3C(C)C)c3nc(-c4ccccc4F)c(F)cc23)cc1] |
| 59 | 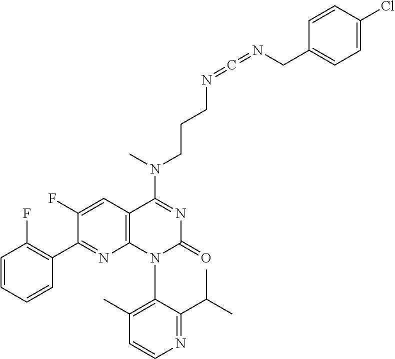 [SMILES: Cc1ccnc(C(C)C)c1-n1c(=O)nc(N(C)CCCN=C=NCc2ccc(Cl)cc2)c2cc(F)c(-c3ccccc3F)nc21] |
| 60 | 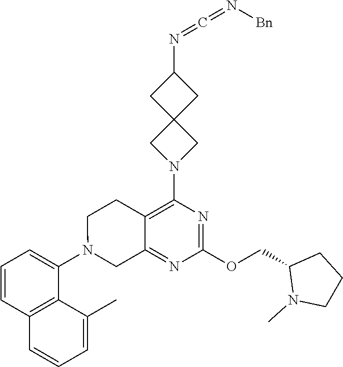 [SMILES: Cc1cccc2cccc(N3CCc4c(nc(OC[C@@H]5CCCN5C)nc4N4CC5(CC(N=C=NCc6ccccc6)C5)C4)C3)c12] |
| 61 | 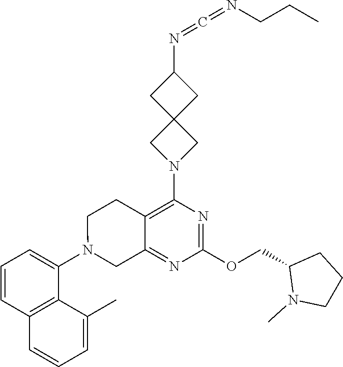 [SMILES: CCCN=C=NC1CC2(C1)CN(c1nc(OC[C@@H]3CCCN3C)nc3c1CCN(c1cccc4cccc(C)c14)C3)C2] |
| 62 | 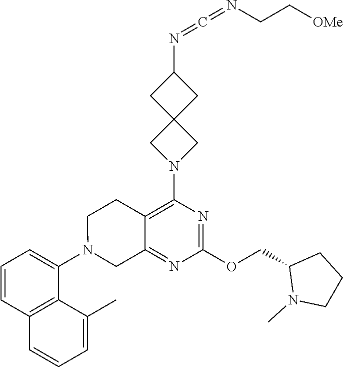 [SMILES: COCCN=C=NC1CC2(C1)CN(c1nc(OC[C@@H]3CCCN3C)nc3c1CCN(c1cccc4cccc(C)c14)C3)C2] |

In some embodiments, a compound of the present invention is selected from Table 2a:

TABLE 2a
Certain Compounds of the Present Invention

| # | Structure |
|---|---|
| 1 | 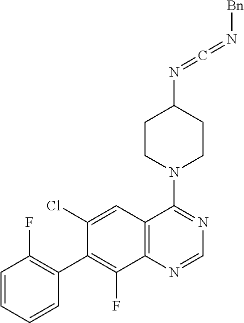 [SMILES: Fc1ccccc1-c1c(Cl)cc2c(N3CCC(N=C=NCc4ccccc4)CC3)ncnc2c1F] |
| 2 | 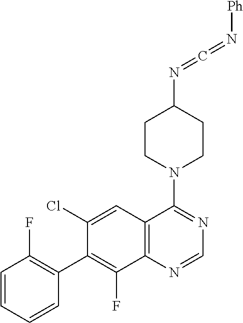 [SMILES: Fc1ccccc1-c1c(Cl)cc2c(N3CCC(N=C=Nc4ccccc4)CC3)ncnc2c1F] |
| 3 | 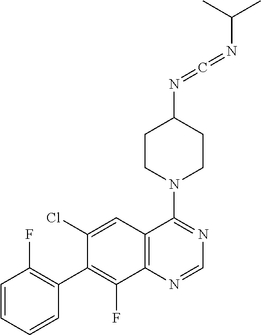 [SMILES: CC(C)N=C=NC1CCN(c2ncnc3c(F)c(-c4ccccc4F)c(Cl)cc23)CC1] |
| 4 | 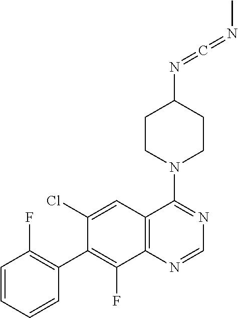 [SMILES: CN=C=NC1CCN(c2ncnc3c(F)c(-c4ccccc4F)c(Cl)cc23)CC1] |
| 7 | 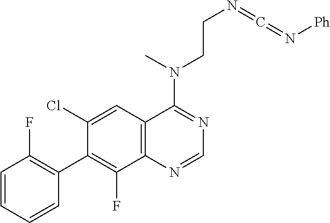 [SMILES: CN(CCN=C=Nc1ccccc1)c1ncnc2c(F)c(-c3ccccc3F)c(Cl)cc12] |
| 8 | 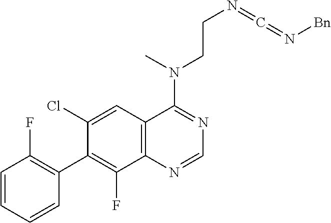 [SMILES: CN(CCN=C=NCc1ccccc1)c1ncnc2c(F)c(-c3ccccc3F)c(Cl)cc12] |
| 9 | 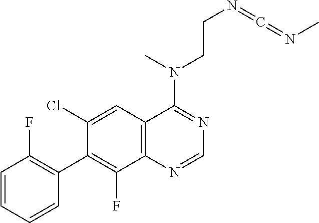 [SMILES: CN=C=NCCN(C)c1ncnc2c(F)c(-c3ccccc3F)c(Cl)cc12] |
| 10 | 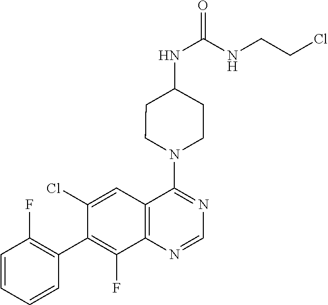 [SMILES: O=C(NCCCl)NC1CCN(c2ncnc3c(F)c(-c4ccccc4F)c(Cl)cc23)CC1] |
| 11 | 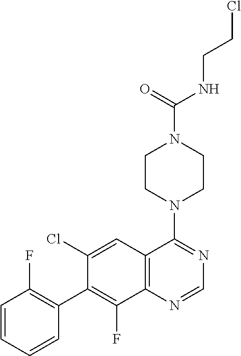 [SMILES: O=C(NCCCl)N1CCN(c2ncnc3c(F)c(-c4ccccc4F)c(Cl)cc23)CC1] |
| 13 | 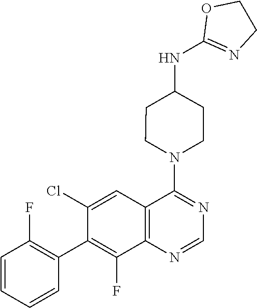 [SMILES: Fc1ccccc1-c1c(Cl)cc2c(N3CCC(NC4=NCCO4)CC3)ncnc2c1F] |
| 14 | 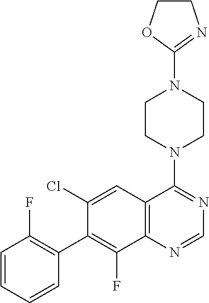 [SMILES: Fc1ccccc1-c1c(Cl)cc2c(N3CCN(C4=NCCO4)CC3)ncnc2c1F] |
| 15 | 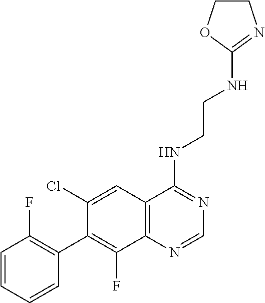 [SMILES: Fc1ccccc1-c1c(Cl)cc2c(NCCNC3=NCCO3)ncnc2c1F] |
| 16 | 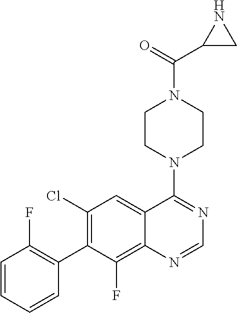 [SMILES: O=C(C1CN1)N1CCN(c2ncnc3c(F)c(-c4ccccc4F)c(Cl)cc23)CC1] |
| 17 | 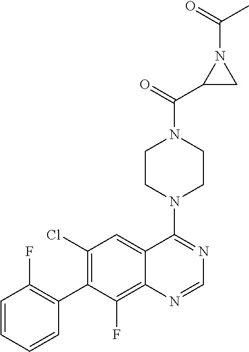 [SMILES: CC(=O)N1CC1C(=O)N1CCN(c2ncnc3c(F)c(-c4ccccc4F)c(Cl)cc23)CC1] |
| 20 | 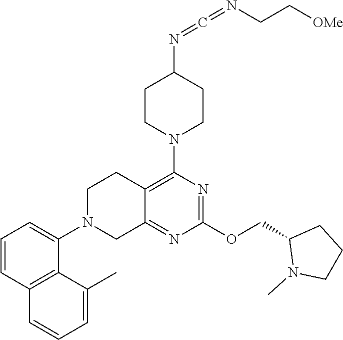 [SMILES: COCCN=C=NC1CCN(c2nc(OC[C@@H]3CCCN3C)nc3c2CCN(c2cccc4cccc(C)c24)C3)CC1] |
| 21 | 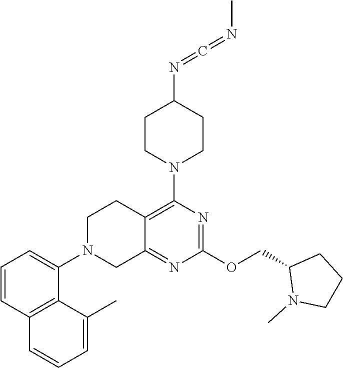 [SMILES: CN=C=NC1CCN(c2nc(OC[C@@H]3CCCN3C)nc3c2CCN(c2cccc4cccc(C)c24)C3)CC1] |
| 22 | 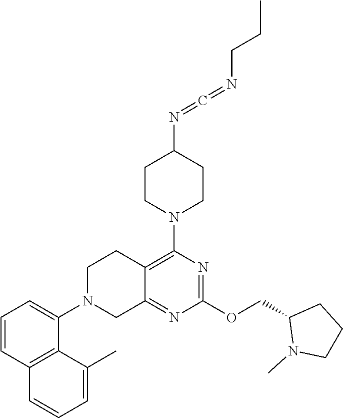 [SMILES: CCCN=C=NC1CCN(c2nc(OC[C@@H]3CCCN3C)nc3c2CCN(c2cccc4cccc(C)c24)C3)CC1] |
| 23 | 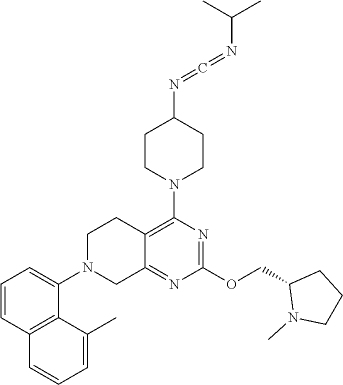 [SMILES: Cc1cccc2cccc(N3CCc4c(nc(OC[C@@H]5CCCN5C)nc4N4CCC(N=C=NC(C)C)CC4)C3)c12] |
| 24 | 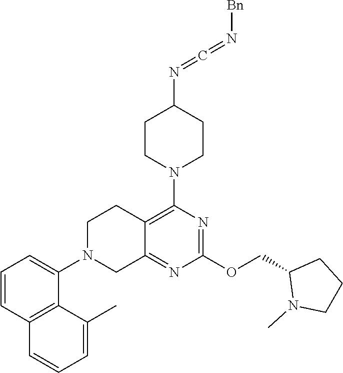 [SMILES: Cc1cccc2cccc(N3CCc4c(nc(OC[C@@H]5CCCN5C)nc4N4CCC(N=C=NCc5ccccc5)CC4)C3)c12] |
| 25 | 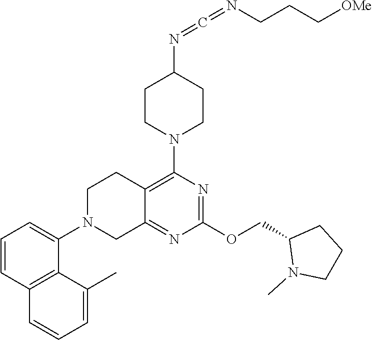 [SMILES: COCCCN=C=NC1CCN(c2nc(OC[C@@H]3CCCN3C)nc3c2CCN(c2cccc4cccc(C)c24)C3)CC1] |
| 26 | 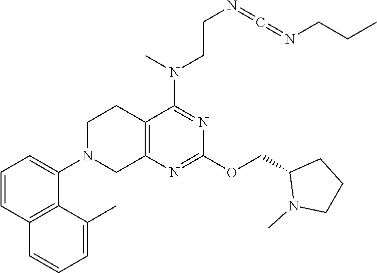 [SMILES: CCCN=C=NCCN(C)c1nc(OC[C@@H]2CCCN2C)nc2c1CCN(c1cccc3cccc(C)c13)C2] |
| 27 | 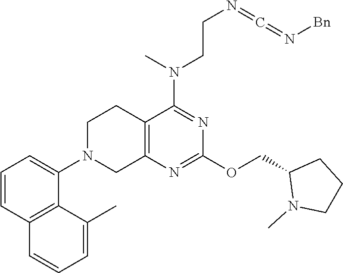 [SMILES: Cc1cccc2cccc(N3CCc4c(nc(OC[C@@H]5CCCN5C)nc4N(C)CCN=C=NCc4ccccc4)C3)c12] |
| 28 | 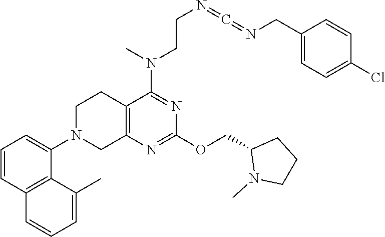 [SMILES: Cc1cccc2cccc(N3CCc4c(nc(OC[C@@H]5CCCN5C)nc4N(C)CCN=C=NCc4ccc(Cl)cc4)C3)c12] |
| 29 | 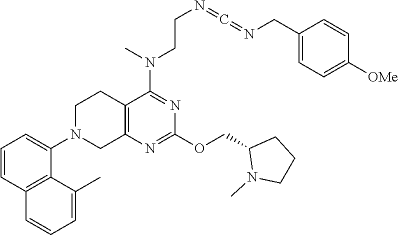 [SMILES: COc1ccc(CN=C=NCCN(C)c2nc(OC[C@@H]3CCCN3C)nc3c2CCN(c2cccc4cccc(C)c24)C3)cc1] |
| 30 | 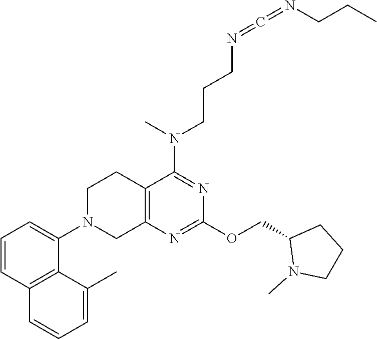 [SMILES: CCCN=C=NCCCN(C)c1nc(OC[C@@H]2CCCN2C)nc2c1CCN(c1cccc3cccc(C)c13)C2] |
| 31 | 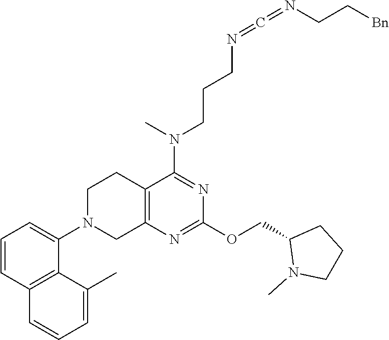 [SMILES: Cc1cccc2cccc(N3CCc4c(nc(OC[C@@H]5CCCN5C)nc4N(C)CCCN=C=NCCCc4ccccc4)C3)c12] |
| 32 | 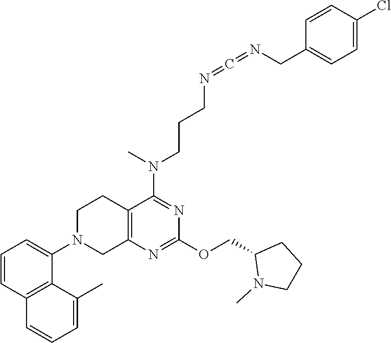 [SMILES: Cc1cccc2cccc(N3CCc4c(nc(OC[C@@H]5CCCN5C)nc4N(C)CCCN=C=NCc4ccc(Cl)cc4)C3)c12] |
| 33 | 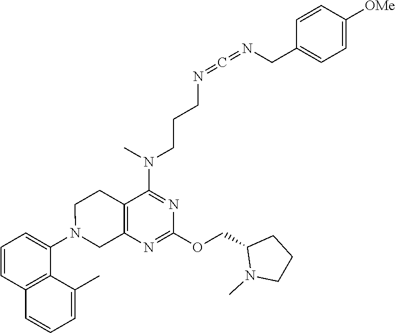 [SMILES: COc1ccc(CN=C=NCCCN(C)c2nc(OC[C@@H]3CCCN3C)nc3c2CCN(c2cccc4cccc(C)c24)C3)cc1] |
| 34 | 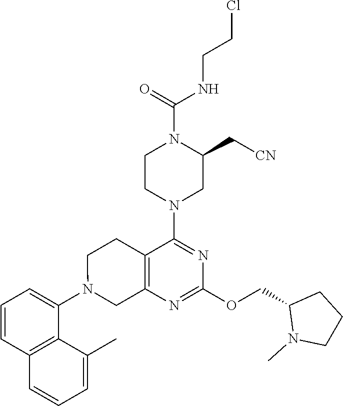 [SMILES: Cc1cccc2cccc(N3CCc4c(nc(OC[C@@H]5CCCN5C)nc4N4CCN(C(=O)NCCCl)[C@@H](CC#N)C4)C3)c12] |
| 35 | 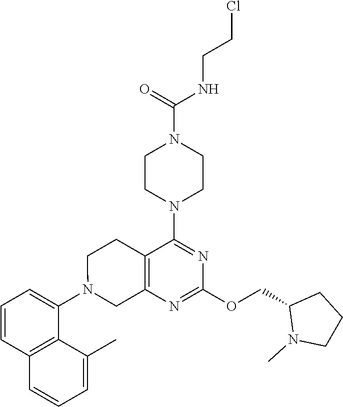 [SMILES: Cc1cccc2cccc(N3CCc4c(nc(OC[C@@H]5CCCN5C)nc4N4CCN(C(=O)NCCCl)CC4)C3)c12] |
| 36 | 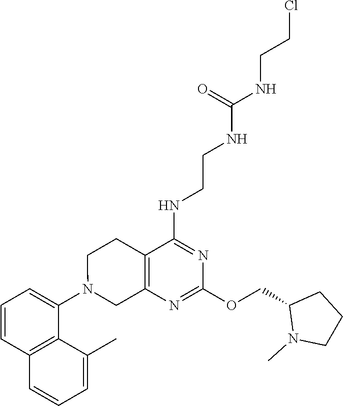 [SMILES: Cc1cccc2cccc(N3CCc4c(nc(OC[C@@H]5CCCN5C)nc4NCCNC(=O)NCCCl)C3)c12] |
| 39 | 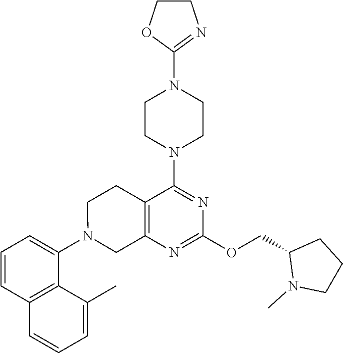 [SMILES: Cc1cccc2cccc(N3CCc4c(nc(OC[C@@H]5CCCN5C)nc4N4CCN(C5=NCCO5)CC4)C3)c12] |
| 40 | 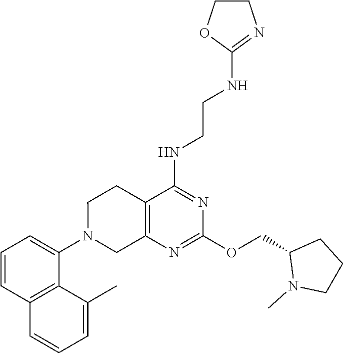 [SMILES: Cc1cccc2cccc(N3CCc4c(nc(OC[C@@H]5CCCN5C)nc4NCCNC4=NCCO4)C3)c12] |
| 42 | 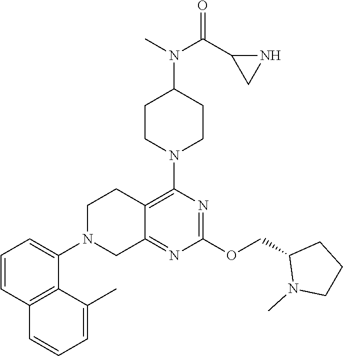 [SMILES: Cc1cccc2cccc(N3CCc4c(nc(OC[C@@H]5CCCN5C)nc4N4CCC(N(C)C(=O)C5CN5)CC4)C3)c12] |
| 43 | 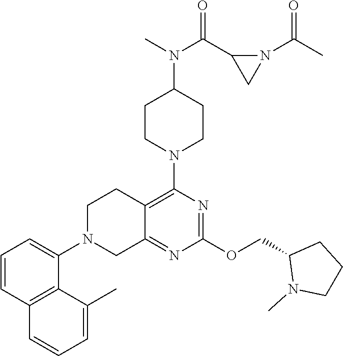 [SMILES: CC(=O)N1CC1C(=O)N(C)C1CCN(c2nc(OC[C@@H]3CCCN3C)nc3c2CCN(c2cccc4cccc(C)c24)C3)CC1] |
| 44 | 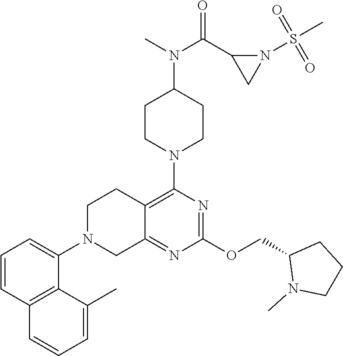 [SMILES: Cc1cccc2cccc(N3CCc4c(nc(OC[C@@H]5CCCN5C)nc4N4CCC(N(C)C(=O)C5CN5S(C)(=O)=O)CC4)C3)c12] |
| 45 | 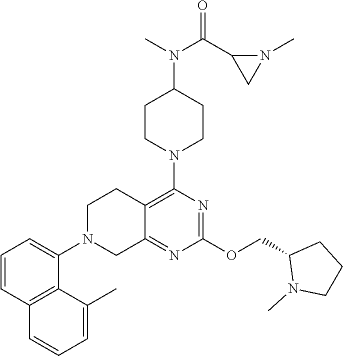 [SMILES: Cc1cccc2cccc(N3CCc4c(nc(OC[C@@H]5CCCN5C)nc4N4CCC(N(C)C(=O)C5CN5C)CC4)C3)c12] |
| 46 | 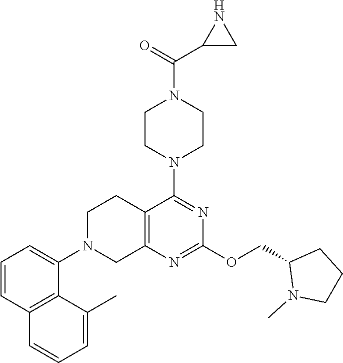 [SMILES: Cc1cccc2cccc(N3CCc4c(nc(OC[C@@H]5CCCN5C)nc4N4CCN(C(=O)C5CN5)CC4)C3)c12] |
| 47 | 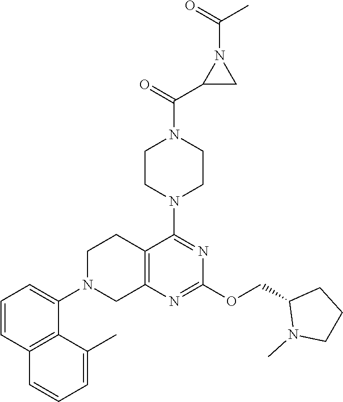 [SMILES: CC(=O)N1CC1C(=O)N1CCN(c2nc(OC[C@@H]3CCCN3C)nc3c2CCN(c2cccc4cccc(C)c24)C3)CC1] |
| 48 | 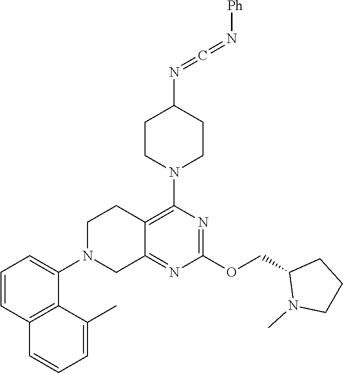 [SMILES: Cc1cccc2cccc(N3CCc4c(nc(OC[C@@H]5CCCN5C)nc4N4CCC(N=C=Nc5ccccc5)CC4)C3)c12] |
| 49 | 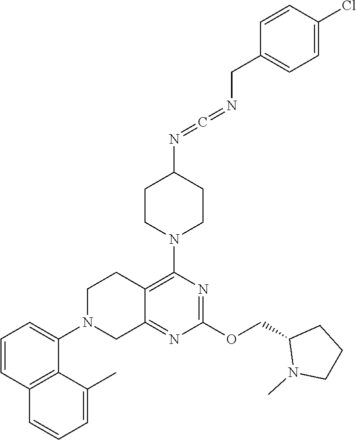 [SMILES: Cc1cccc2cccc(N3CCc4c(nc(OC[C@@H]5CCCN5C)nc4N4CCC(N=C=NCc5ccc(Cl)cc5)CC4)C3)c12] |
| 50 | 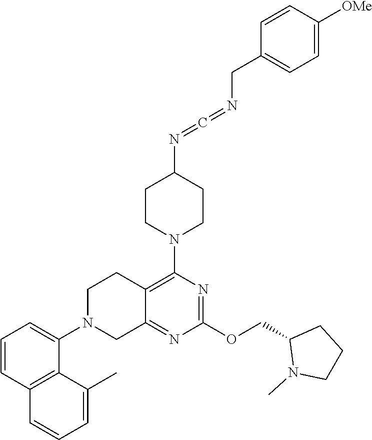 [SMILES: COc1ccc(CN=C=NC2CCN(c3nc(OC[C@@H]4CCCN4C)nc4c3CCN(c3cccc5cccc(C)c35)C4)CC2)cc1] |
| 51 | 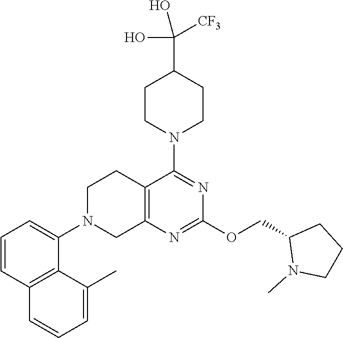 [SMILES: Cc1cccc2cccc(N3CCc4c(nc(OC[C@@H]5CCCN5C)nc4N4CCC(C(O)(O)C(F)(F)F)CC4)C3)c12] |
| 52 | 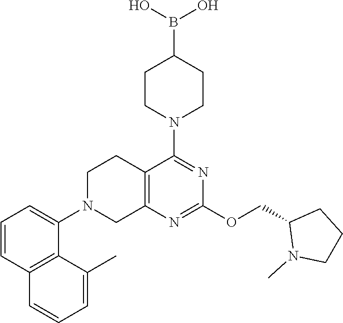 [SMILES: Cc1cccc2cccc(N3CCc4c(nc(OC[C@@H]5CCCN5C)nc4N4CCC(B(O)O)CC4)C3)c12] |
| 53 | 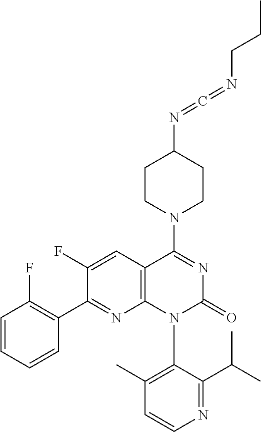 [SMILES: CCCN=C=NC1CCN(c2nc(=O)n(-c3c(C)ccnc3C(C)C)c3nc(-c4ccccc4F)c(F)cc23)CC1] |
| 54 | 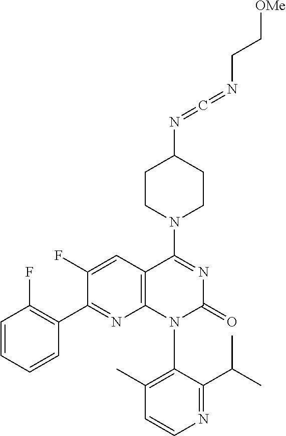 [SMILES: COCCN=C=NC1CCN(c2nc(=O)n(-c3c(C)ccnc3C(C)C)c3nc(-c4ccccc4F)c(F)cc23)CC1] |
| 55 | 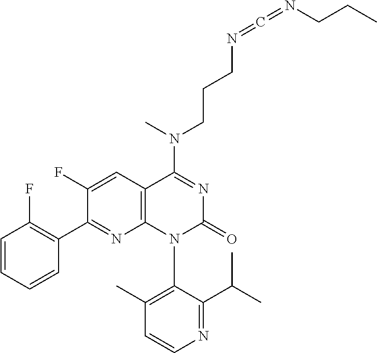 [SMILES: CCCN=C=NCCCN(C)c1nc(=O)n(-c2c(C)ccnc2C(C)C)c2nc(-c3ccccc3F)c(F)cc12] |
| 56 | 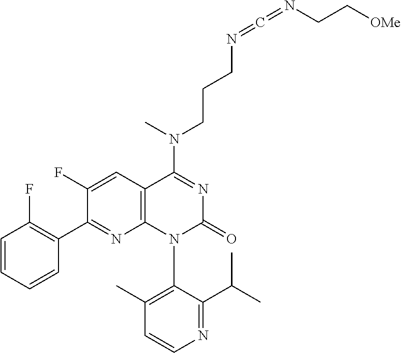 [SMILES: COCCN=C=NCCCN(C)c1nc(=O)n(-c2c(C)ccnc2C(C)C)c2nc(-c3ccccc3F)c(F)cc12] |
| 57 | 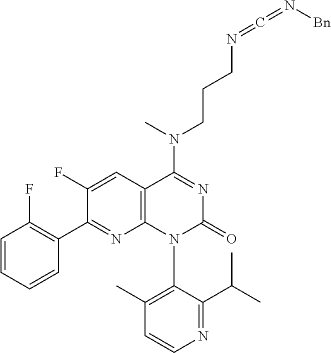 [SMILES: Cc1ccnc(C(C)C)c1-n1c(=O)nc(N(C)CCCN=C=NCc2ccccc2)c2cc(F)c(-c3ccccc3F)nc21] |
| 58 | 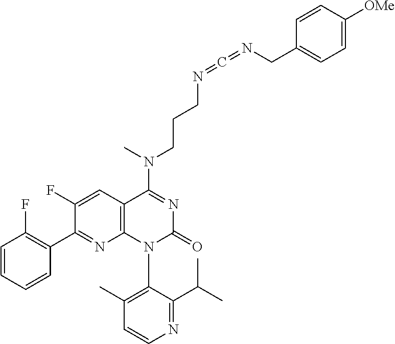 [SMILES: COc1ccc(CN=C=NCCCN(C)c2nc(=O)n(-c3c(C)ccnc3C(C)C)c3nc(-c4ccccc4F)c(F)cc23)cc1] |
| 59 | 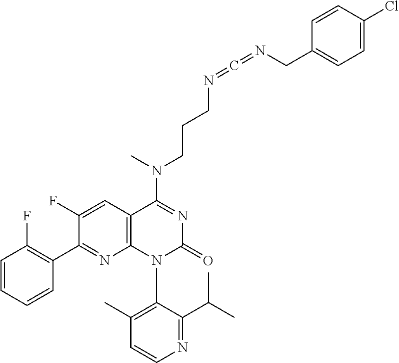 [SMILES: Cc1ccnc(C(C)C)c1-n1c(=O)nc(N(C)CCCN=C=NCc2ccc(Cl)cc2)c2cc(F)c(-c3ccccc3F)nc21] |
| 60 | 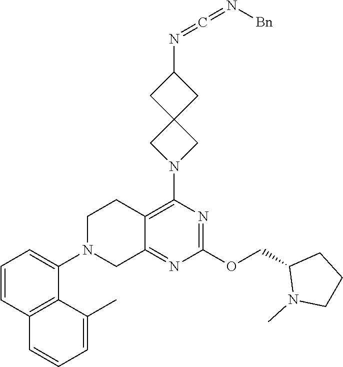 [SMILES: Cc1cccc2cccc(N3CCc4c(nc(OC[C@@H]5CCCN5C)nc4N4CC5(CC(N=C=NCc6ccccc6)C5)C4)C3)c12] |
| 61 | 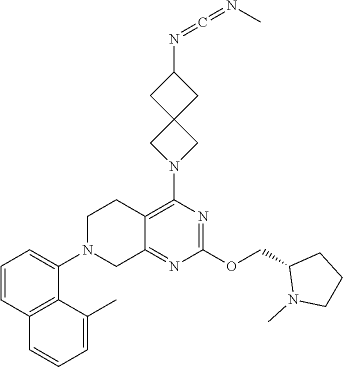 [SMILES: CN=C=NC1CC2(C1)CN(c1nc(OC[C@@H]3CCCN3C)nc3c1CCN(c1cccc4cccc(C)c14)C3)C2] |
| 62 | 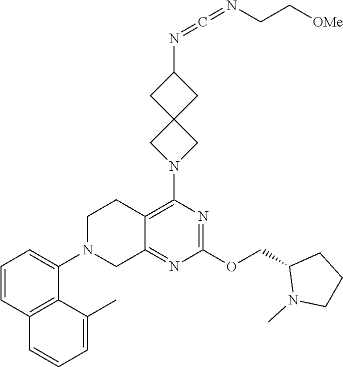 [SMILES: COCCN=C=NC1CC2(C1)CN(c1nc(OC[C@@H]3CCCN3C)nc3c1CCN(c1cccc4cccc(C)c14)C3)C2] |

In some embodiments, a compound of the present invention is selected from Table 2b:

TABLE 2b
Certain Compounds of the Present Invention

| # | Structure |
|---|---|
| 63 | 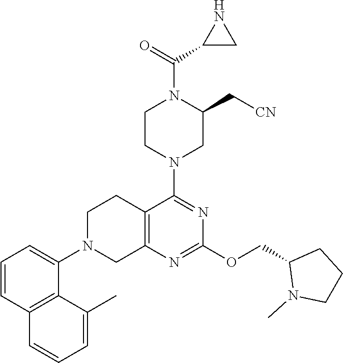 [SMILES: Cc1cccc2cccc(N3CCc4c(nc(OC[C@@H]5CCCN5C)nc4N4CCN(C(=O)[C@H]5CN5)[C@@H](CC#N)C4)C3)c12] |
| 64 | 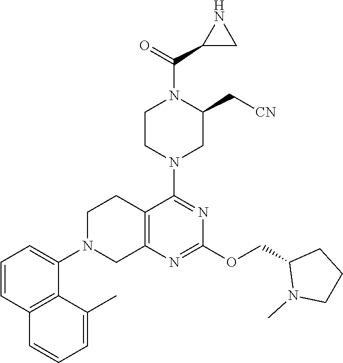 [SMILES: Cc1cccc2cccc(N3CCc4c(nc(OC[C@@H]5CCCN5C)nc4N4CCN(C(=O)[C@@H]5CN5)[C@@H](CC#N)C4)C3)c12] |
| 65 | 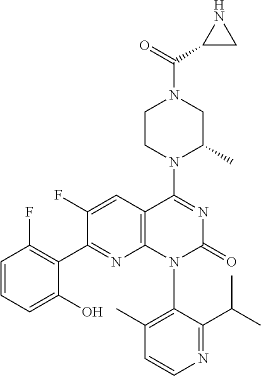 [SMILES: Cc1ccnc(C(C)C)c1-n1c(=O)nc(N2CCN(C(=O)[C@H]3CN3)C[C@@H]2C)c2cc(F)c(-c3c(O)cccc3F)nc21] |
| 66 | 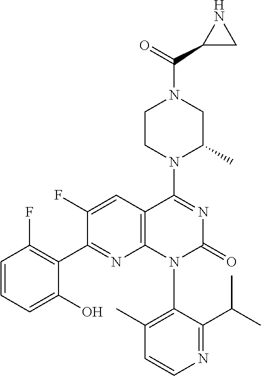 [SMILES: Cc1ccnc(C(C)C)c1-n1c(=O)nc(N2CCN(C(=O)[C@@H]3CN3)C[C@@H]2C)c2cc(F)c(-c3c(O)cccc3F)nc21] |
| 67 | 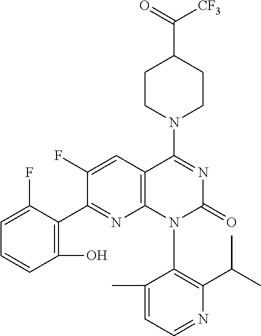 [SMILES: Cc1ccnc(C(C)C)c1-n1c(=O)nc(N2CCC(C(=O)C(F)(F)F)CC2)c2cc(F)c(-c3c(O)cccc3F)nc21] |
| 68 | 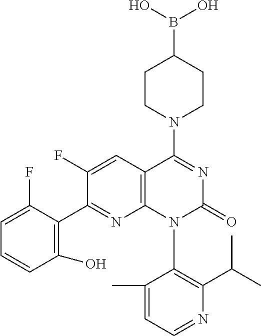 [SMILES: Cc1ccnc(C(C)C)c1-n1c(=O)nc(N2CCC(B(O)O)CC2)c2cc(F)c(-c3c(O)cccc3F)nc21] |
| 69 | 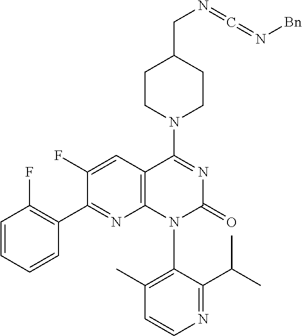 [SMILES: Cc1ccnc(C(C)C)c1-n1c(=O)nc(N2CCC(CN=C=NCc3ccccc3)CC2)c2cc(F)c(-c3ccccc3F)nc21] |
| 70 | 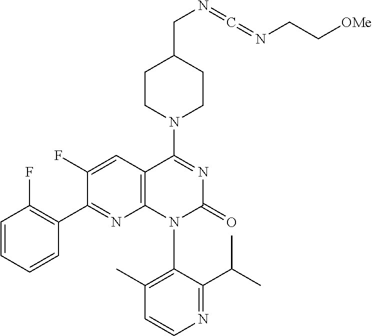 [SMILES: COCCN=C=NCC1CCN(c2nc(=O)n(-c3c(C)ccnc3C(C)C)c3nc(-c4ccccc4F)c(F)cc23)CC1] |
| 71 | 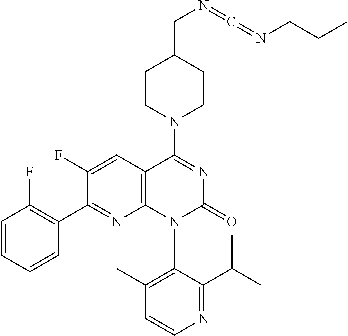 [SMILES: CCCN=C=NCC1CCN(c2nc(=O)n(-c3c(C)ccnc3C(C)C)c3nc(-c4ccccc4F)c(F)cc23)CC1] |
| 72 | 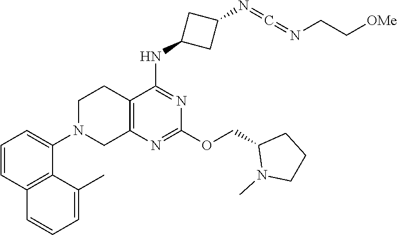 [SMILES: COCCN=C=N[C@H]1C[C@H](Nc2nc(OC[C@@H]3CCCN3C)nc3c2CCN(c2cccc4cccc(C)c24)C3)C1] |
| 73 | 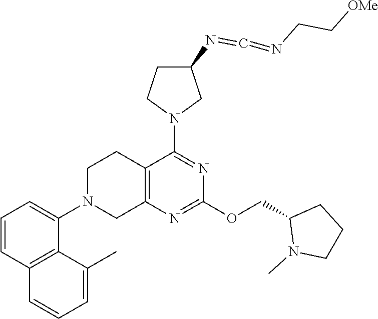 [SMILES: COCCN=C=N[C@@H]1CCN(c2nc(OC[C@@H]3CCCN3C)nc3c2CCN(c2cccc4cccc(C)c24)C3)C1] |
| 74 | 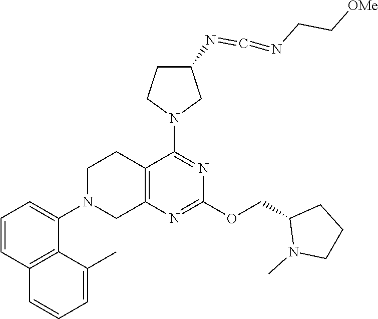 [SMILES: COCCN=C=N[C@H]1CCN(c2nc(OC[C@@H]3CCCN3C)nc3c2CCN(c2cccc4cccc(C)c24)C3)C1] |
| 75 | 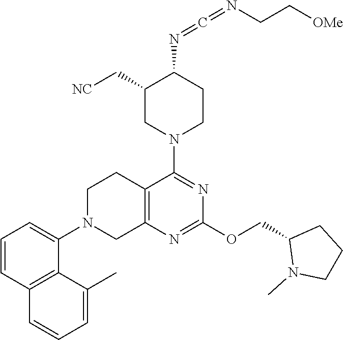 [SMILES: COCCN=C=N[C@@H]1CCN(c2nc(OC[C@@H]3CCCN3C)nc3c2CCN(c2cccc4cccc(C)c24)C3)C[C@@H]1CC#N] |
| 76 | 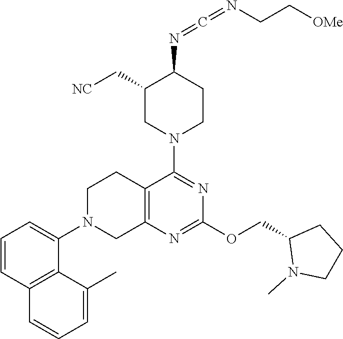 [SMILES: COCCN=C=N[C@H]1CCN(c2nc(OC[C@@H]3CCCN3C)nc3c2CCN(c2cccc4cccc(C)c24)C3)C[C@@H]1CC#N] |
| 77 | 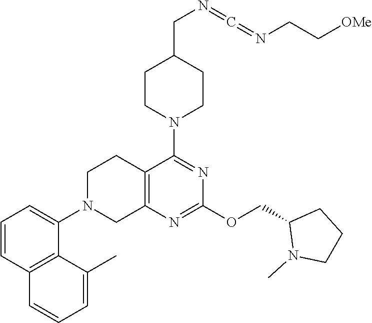 [SMILES: COCCN=C=NCC1CCN(c2nc(OC[C@@H]3CCCN3C)nc3c2CCN(c2cccc4cccc(C)c24)C3)CC1] |
| 78 | 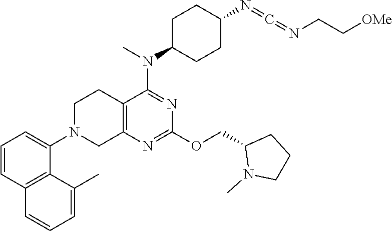 [SMILES: COCCN=C=N[C@H]1CC[C@H](N(C)c2nc(OC[C@@H]3CCCN3C)nc3c2CCN(c2cccc4cccc(C)c24)C3)CC1] |
| 79 | 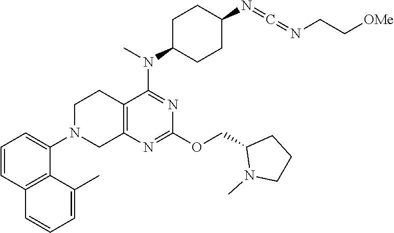 [SMILES: COCCN=C=N[C@H]1CC[C@@H](N(C)c2nc(OC[C@@H]3CCCN3C)nc3c2CCN(c2cccc4cccc(C)c24)C3)CC1] |
| 80 | 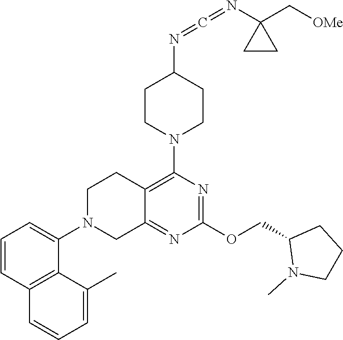 [SMILES: COCC1(N=C=NC2CCN(c3nc(OC[C@@H]4CCCN4C)nc4c3CCN(c3cccc5cccc(C)c35)C4)CC2)CC1] |
| 81 | 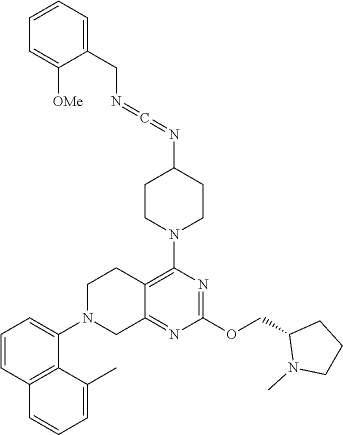 [SMILES: COc1ccccc1CN=C=NC1CCN(c2nc(OC[C@@H]3CCCN3C)nc3c2CCN(c2cccc4cccc(C)c24)C3)CC1] |
| 82 | 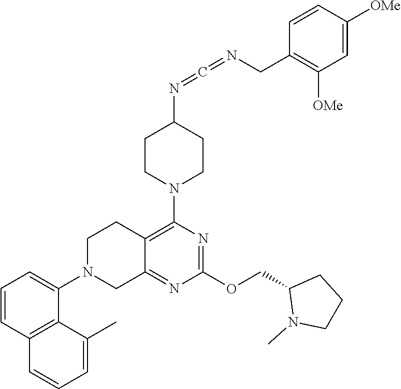 [SMILES: COc1ccc(CN=C=NC2CCN(c3nc(OC[C@@H]4CCCN4C)nc4c3CCN(c3cccc5cccc(C)c35)C4)CC2)c(OC)c1] |
| 83 | 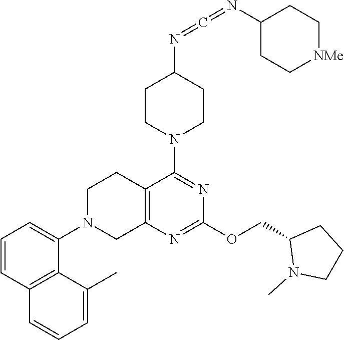 [SMILES: Cc1cccc2cccc(N3CCc4c(nc(OC[C@@H]5CCCN5C)nc4N4CCC(N=C=NC5CCN(C)CC5)CC4)C3)c12] |
| 84 | 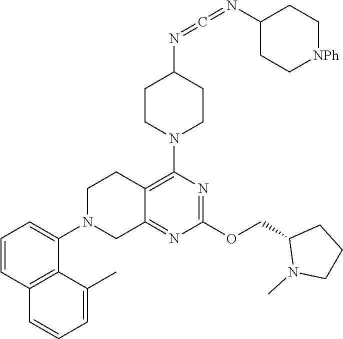 |
| 85 | 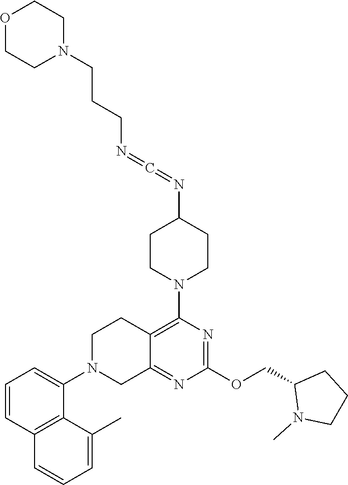 [SMILES: Cc1cccc2cccc(N3CCc4c(nc(OC[C@@H]5CCCN5C)nc4N4CCC(N=C=NCCCN5CCOCC5)CC4)C3)c12] |
| 86 | 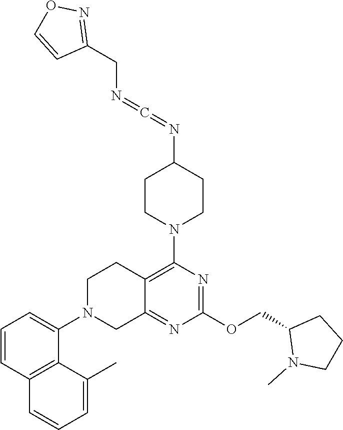 [SMILES: Cc1cccc2cccc(N3CCc4c(nc(OC[C@@H]5CCCN5C)nc4N4CCC(N=C=NCc5ccon5)CC4)C3)c12] |
| 87 | 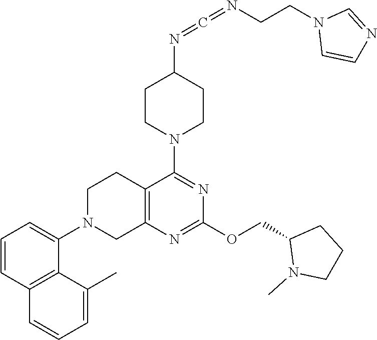 [SMILES: Cc1cccc2cccc(N3CCc4c(nc(OC[C@@H]5CCCN5C)nc4N4CCC(N=C=NCCn5ccnc5)CC4)C3)c12] |
| 88 | 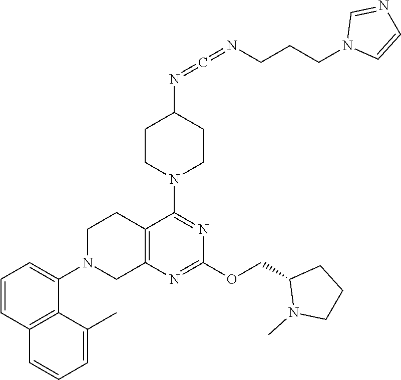 [SMILES: Cc1cccc2cccc(N3CCc4c(nc(OC[C@@H]5CCCN5C)nc4N4CCC(N=C=NCCCn5ccnc5)CC4)C3)c12] |
| 89 | 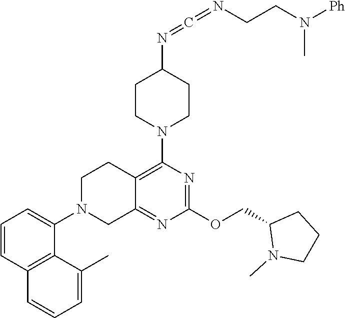 [SMILES: Cc1cccc2cccc(N3CCc4c(nc(OC[C@@H]5CCCN5C)nc4N4CCC(N=C=NCCN(C)c5ccccc5)CC4)C3)c12] |
| 90 | 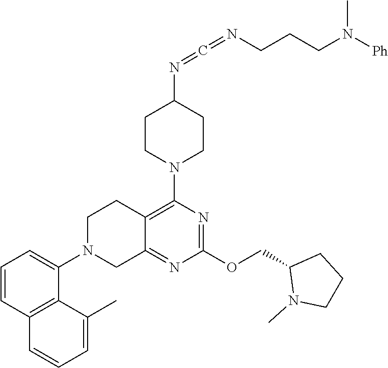 [SMILES: Cc1cccc2cccc(N3CCc4c(nc(OC[C@@H]5CCCN5C)nc4N4CCC(N=C=NCCCN(C)c5ccccc5)CC4)C3)c12] |
| 91 | 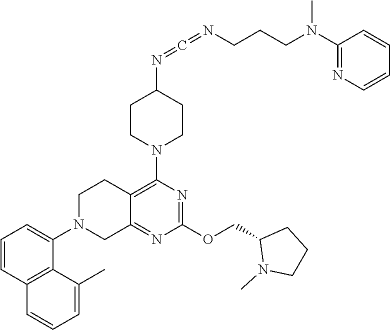 [SMILES: Cc1cccc2cccc(N3CCc4c(nc(OC[C@@H]5CCCN5C)nc4N4CCC(N=C=NCCCN(C)c5ccccn5)CC4)C3)c12] |
| 92 | 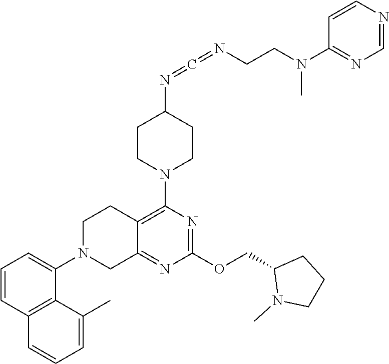 [SMILES: Cc1cccc2cccc(N3CCc4c(nc(OC[C@@H]5CCCN5C)nc4N4CCC(N=C=NCCN(C)c5ccncn5)CC4)C3)c12] |
| 93 | 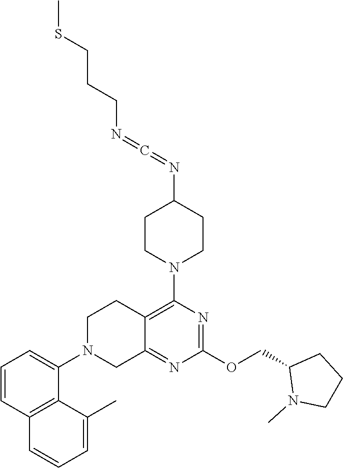 [SMILES: CSCCCN=C=NC1CCN(c2nc(OC[C@@H]3CCCN3C)nc3c2CCN(c2cccc4cccc(C)c24)C3)CC1] |
| 94 | 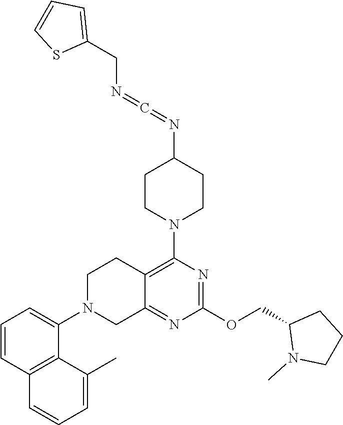 [SMILES: Cc1cccc2cccc(N3CCc4c(nc(OC[C@@H]5CCCN5C)nc4N4CCC(N=C=NCc5cccs5)CC4)C3)c12] |
| 95 | 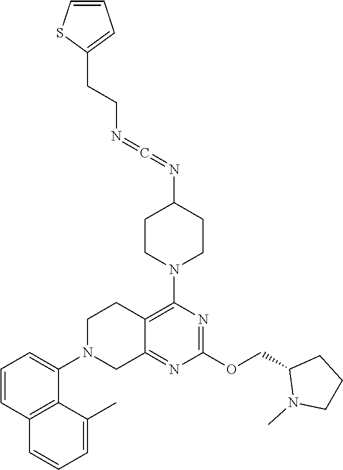 [SMILES: Cc1cccc2cccc(N3CCc4c(nc(OC[C@@H]5CCCN5C)nc4N4CCC(N=C=NCCc5cccs5)CC4)C3)c12] |

In some embodiments, a compound of the present invention is selected from Table 2c:

TABLE 2c
Certain Compounds of the Present Invention

| # | Structure |
|---|---|
| 96 | 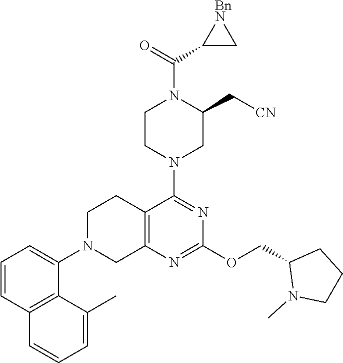 [SMILES: Cc1cccc2cccc(N3CCc4c(nc(OC[C@@H]5CCCN5C)nc4N4CCN(C(=O)[C@H]5CN5Cc5ccccc5)[C@@H](CC#N)C4)C3)c12] |
| 97 | 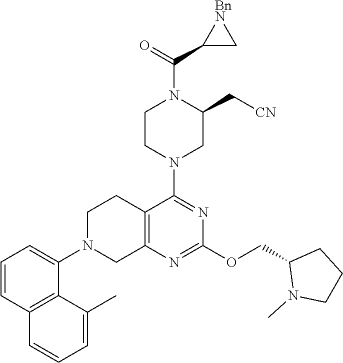 [SMILES: Cc1cccc2cccc(N3CCc4c(nc(OC[C@@H]5CCCN5C)nc4N4CCN(C(=O)[C@@H]5CN5Cc5ccccc5)[C@@H](CC#N)C4)C3)c12] |
| 98 | 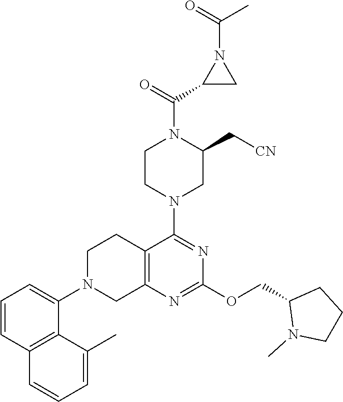 [SMILES: CC(=O)N1C[C@@H]1C(=O)N1CCN(c2nc(OC[C@@H]3CCCN3C)nc3c2CCN(c2cccc4cccc(C)c24)C3)C[C@@H]1CC#N] |
| 99 | 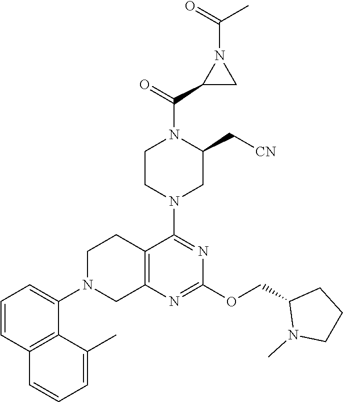 [SMILES: CC(=O)N1C[C@H]1C(=O)N1CCN(c2nc(OC[C@@H]3CCCN3C)nc3c2CCN(c2cccc4cccc(C)c24)C3)C[C@@H]1CC#N] |
| 100 | 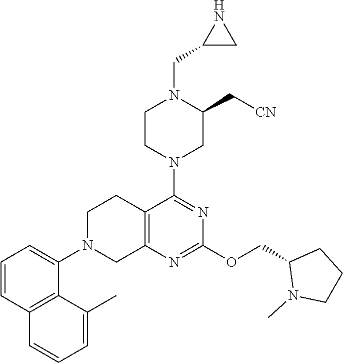 [SMILES: Cc1cccc2cccc(N3CCc4c(nc(OC[C@@H]5CCCN5C)nc4N4CCN(C[C@H]5CN5)[C@@H](CC#N)C4)C3)c12] |
| 101 | 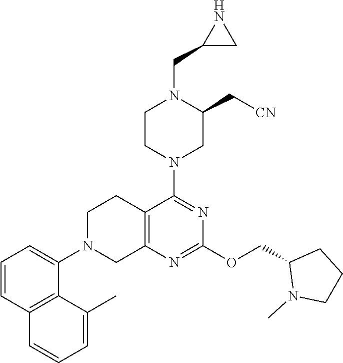 [SMILES: Cc1cccc2cccc(N3CCc4c(nc(OC[C@@H]5CCCN5C)nc4N4CCN(C[C@@H]5CN5)[C@@H](CC#N)C4)C3)c12] |
| 102 | 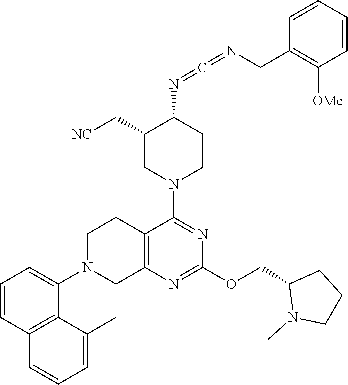 [SMILES: COc1ccccc1CN=C=N[C@@H]1CCN(c2nc(OC[C@@H]3CCCN3C)nc3c2CCN(c2cccc4cccc(C)c24)C3)C[C@@H]1CC#N] |
| 103 | 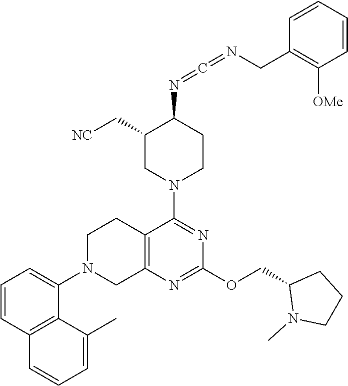 [SMILES: COc1ccccc1CN=C=N[C@H]1CCN(c2nc(OC[C@@H]3CCCN3C)nc3c2CCN(c2cccc4cccc(C)c24)C3)C[C@@H]1CC#N] |
| 104 | 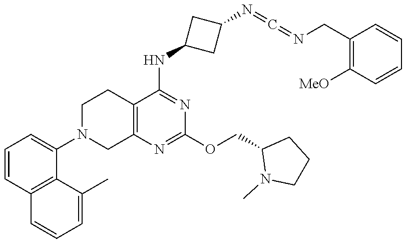 [SMILES: COc1ccccc1CN=C=N[C@H]1C[C@H](Nc2nc(OC[C@@H]3CCCN3C)nc3c2CCN(c2cccc4cccc(C)c24)C3)C1] |

In some embodiments, a compound of the present invention is selected from Table 2d:

TABLE 2d
Certain Compounds of the Present Invention

| # | Structure |
|---|---|
| 105 | 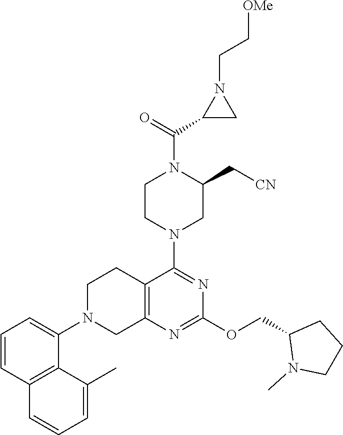 [SMILES: COCCN1C[C@@H]1C(=O)N1CCN(c2nc(OC[C@@H]3CCCN3C)nc3c2CCN(c2cccc4cccc(C)c24)C3)C[C@@H]1CC#N] |
| 106 | 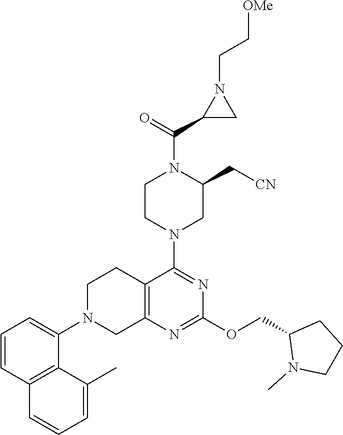 [SMILES: COCCN1C[C@H]1C(=O)N1CCN(c2nc(OC[C@@H]3CCCN3C)nc3c2CCN(c2cccc4cccc(C)c24)C3)C[C@@H]1CC#N] |
| 107 | 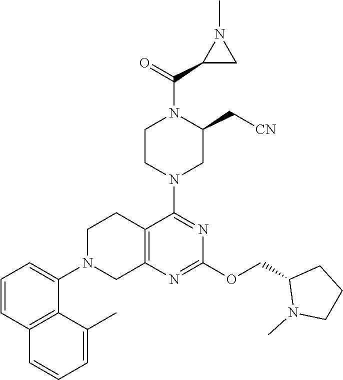 [SMILES: Cc1cccc2cccc(N3CCc4c(nc(OC[C@@H]5CCCN5C)nc4N4CCN(C(=O)[C@@H]5CN5C)[C@@H](CC#N)C4)C3)c12] |
| 108 | 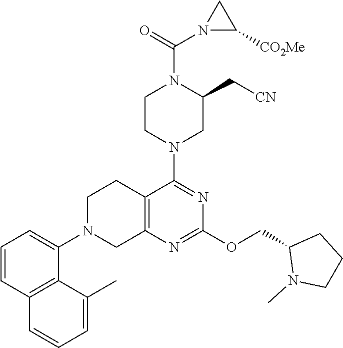 [SMILES: COC(=O)[C@H]1CN1C(=O)N1CCN(c2nc(OC[C@@H]3CCCN3C)nc3c2CCN(c2cccc4cccc(C)c24)C3)C[C@@H]1CC#N] |
| 109 | 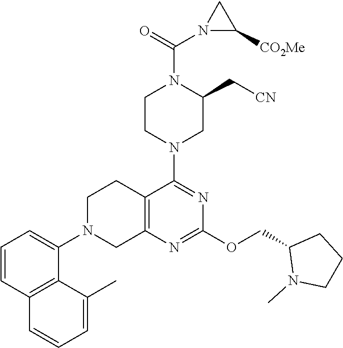 [SMILES: COC(=O)[C@@H]1CN1C(=O)N1CCN(c2nc(OC[C@@H]3CCCN3C)nc3c2CCN(c2cccc4cccc(C)c24)C3)C[C@@H]1CC#N] |
| 110 | 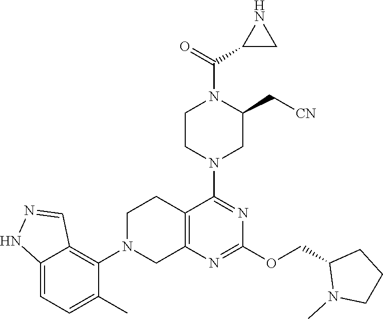 [SMILES: Cc1ccc2[nH]ncc2c1N1CCc2c(nc(OC[C@@H]3CCCN3C)nc2N2CCN(C(=O)[C@H]3CN3)[C@@H](CC#N)C2)C1] |
| 111 | 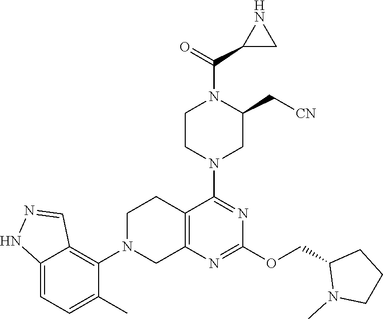 [SMILES: Cc1ccc2[nH]ncc2c1N1CCc2c(nc(OC[C@@H]3CCCN3C)nc2N2CCN(C(=O)[C@@H]3CN3)[C@@H](CC#N)C2)C1] |
| 112 | 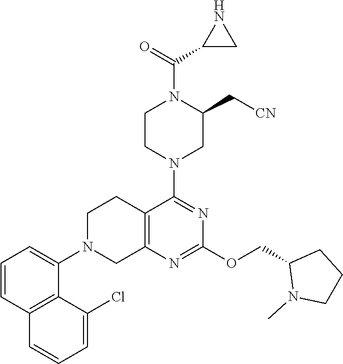 [SMILES: CN1CCC[C@H]1COc1nc2c(c(N3CCN(C(=O)[C@H]4CN4)[C@@H](CC#N)C3)n1)CCN(c1cccc3cccc(Cl)c13)C2] |
| 113 | 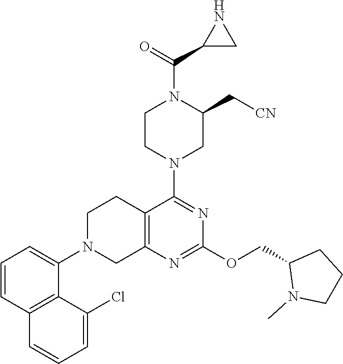 [SMILES: CN1CCC[C@H]1COc1nc2c(c(N3CCN(C(=O)[C@@H]4CN4)[C@@H](CC#N)C3)n1)CCN(c1cccc3cccc(Cl)c13)C2] |
| 114 | 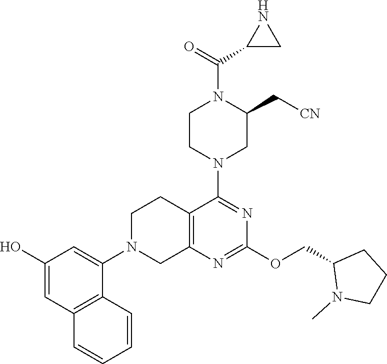 [SMILES: CN1CCC[C@H]1COc1nc2c(c(N3CCN(C(=O)[C@H]4CN4)[C@@H](CC#N)C3)n1)CCN(c1cc(O)cc3ccccc13)C2] |
| 115 | 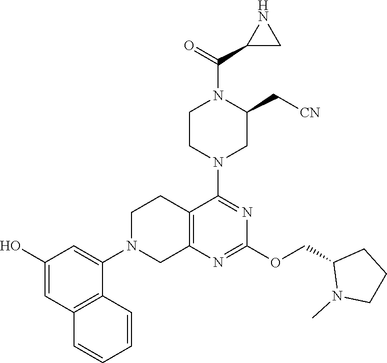 [SMILES: CN1CCC[C@H]1COc1nc2c(c(N3CCN(C(=O)[C@@H]4CN4)[C@@H](CC#N)C3)n1)CCN(c1cc(O)cc3ccccc13)C2] |
| 116 | 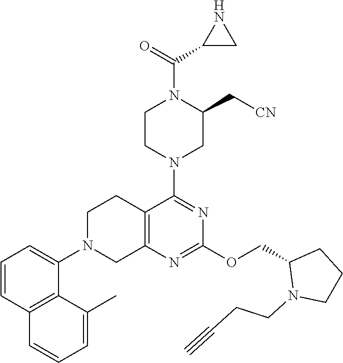 [SMILES: C#CCCN1CCC[C@H]1COc1nc2c(c(N3CCN(C(=O)[C@H]4CN4)[C@@H](CC#N)C3)n1)CCN(c1cccc3cccc(C)c13)C2] |
| 117 | 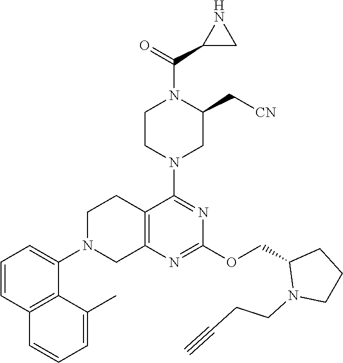 [SMILES: C#CCCN1CCC[C@H]1COc1nc2c(c(N3CCN(C(=O)[C@@H]4CN4)[C@@H](CC#N)C3)n1)CCN(c1cccc3cccc(C)c13)C2] |
| 118 | 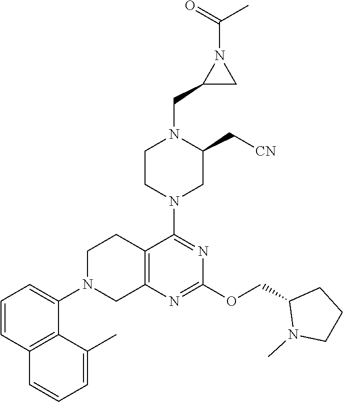 [SMILES: CC(=O)N1C[C@H]1CN1CCN(c2nc(OC[C@@H]3CCCN3C)nc3c2CCN(c2cccc4cccc(C)c24)C3)C[C@@H]1CC#N] |
| 119 | 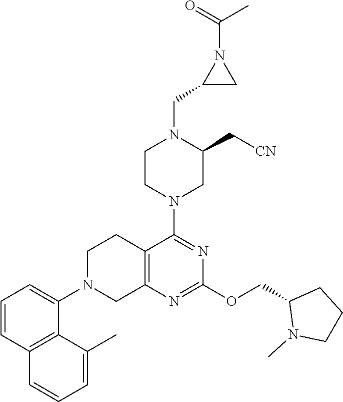 [SMILES: CC(=O)N1C[C@@H]1CN1CCN(c2nc(OC[C@@H]3CCCN3C)nc3c2CCN(c2cccc4cccc(C)c24)C3)C[C@@H]1CC#N] |
| 120 | 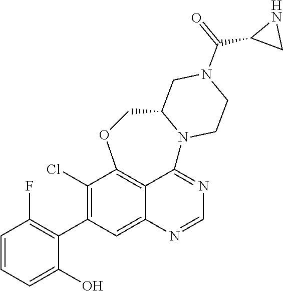 [SMILES: O=C([C@H]1CN1)N1CCN2c3ncnc4cc(-c5c(O)cccc5F)c(Cl)c(c34)OC[C@@H]2C1] |
| 121 | 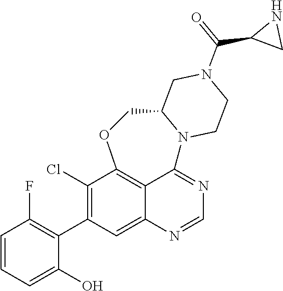 [SMILES: O=C([C@@H]1CN1)N1CCN2c3ncnc4cc(-c5c(O)cccc5F)c(Cl)c(c34)OC[C@@H]2C1] |
| 122 | 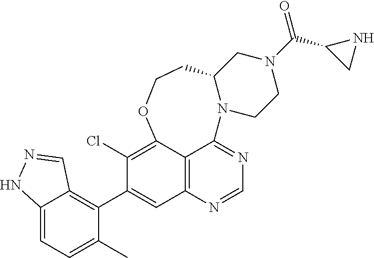 [SMILES: Cc1ccc2[nH]ncc2c1-c1cc2ncnc3c2c(c1Cl)OCC[C@@H]1CN(C(=O)[C@H]2CN2)CCN31] |
| 123 | 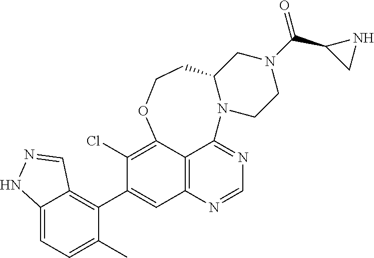 [SMILES: Cc1ccc2[nH]ncc2c1-c1cc2ncnc3c2c(c1Cl)OCC[C@@H]1CN(C(=O)[C@@H]2CN2)CCN31] |
| 124 | 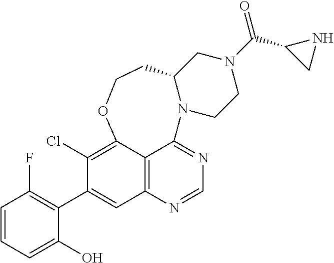 [SMILES: O=C([C@H]1CN1)N1CCN2c3ncnc4cc(-c5c(O)cccc5F)c(Cl)c(c34)OCC[C@@H]2C1] |
| 125 | 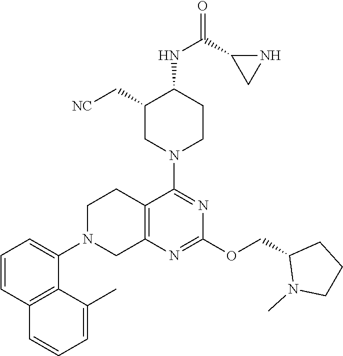 [SMILES: Cc1cccc2cccc(N3CCc4c(nc(OC[C@@H]5CCCN5C)nc4N4CC[C@@H](NC(=O)[C@H]5CN5)[C@@H](CC#N)C4)C3)c12] |
| 126 | 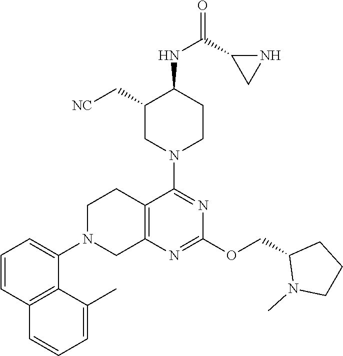 [SMILES: Cc1cccc2cccc(N3CCc4c(nc(OC[C@@H]5CCCN5C)nc4N4CC[C@H](NC(=O)[C@H]5CN5)[C@@H](CC#N)C4)C3)c12] |
| 127 | 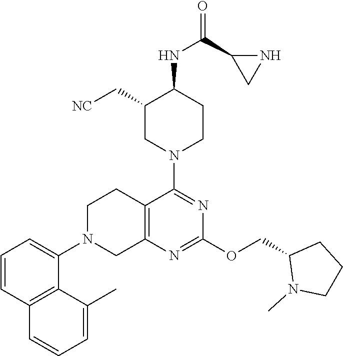 [SMILES: Cc1cccc2cccc(N3CCc4c(nc(OC[C@@H]5CCCN5C)nc4N4CC[C@H](NC(=O)[C@@H]5CN5)[C@@H](CC#N)C4)C3)c12] |
| 128 | 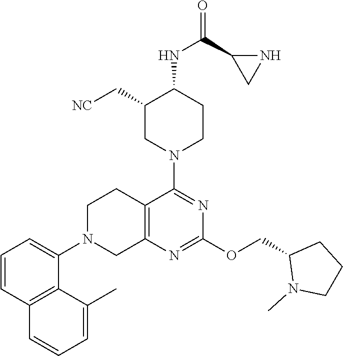 [SMILES: Cc1cccc2cccc(N3CCc4c(nc(OC[C@@H]5CCCN5C)nc4N4CC[C@@H](NC(=O)[C@@H]5CN5)[C@@H](CC#N)C4)C3)c12] |
| 129 | 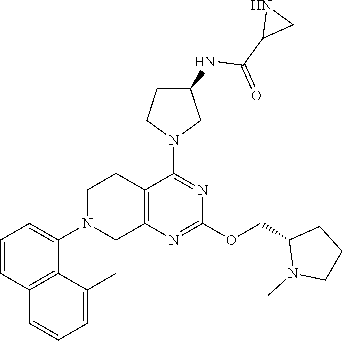 [SMILES: Cc1cccc2cccc(N3CCc4c(nc(OC[C@@H]5CCCN5C)nc4N4CC[C@@H](NC(=O)C5CN5)C4)C3)c12] |
| 130 | 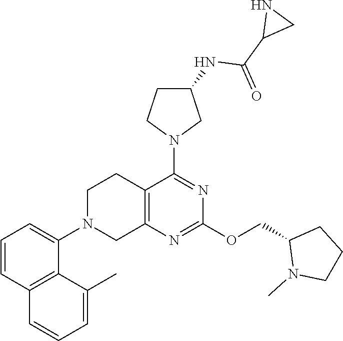 [SMILES: Cc1cccc2cccc(N3CCc4c(nc(OC[C@@H]5CCCN5C)nc4N4CC[C@H](NC(=O)C5CN5)C4)C3)c12] |
| 131 | 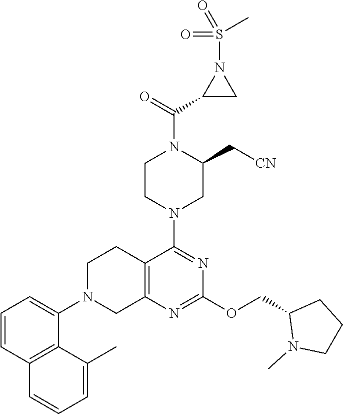 [SMILES: Cc1cccc2cccc(N3CCc4c(nc(OC[C@@H]5CCCN5C)nc4N4CCN(C(=O)[C@H]5CN5S(C)(=O)=O)[C@@H](CC#N)C4)C3)c12] |
| 132 | 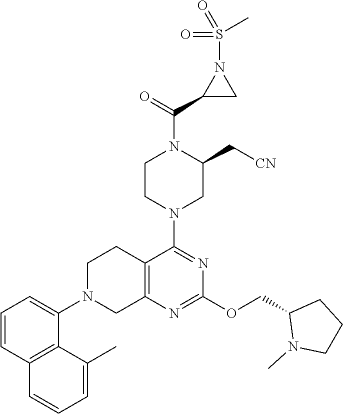 [SMILES: Cc1cccc2cccc(N3CCc4c(nc(OC[C@@H]5CCCN5C)nc4N4CCN(C(=O)[C@@H]5CN5S(C)(=O)=O)[C@@H](CC#N)C4)C3)c12] |
| 133 | 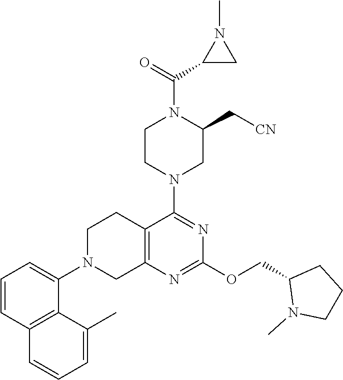 [SMILES: Cc1cccc2cccc(N3CCc4c(nc(OC[C@@H]5CCCN5C)nc4N4CCN(C(=O)[C@H]5CN5C)[C@@H](CC#N)C4)C3)c12] |
| 134 | 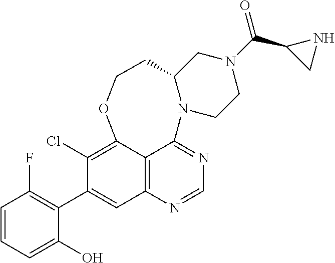 [SMILES: O=C([C@@H]1CN1)N1CCN2c3ncnc4cc(-c5c(O)cccc5F)c(Cl)c(c34)OCC[C@@H]2C1] |
| 135 | 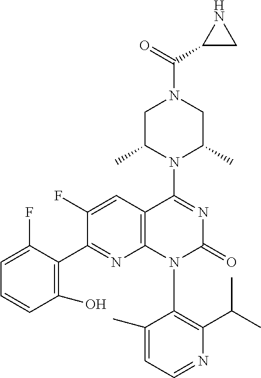 [SMILES: Cc1ccnc(C(C)C)c1-n1c(=O)nc(N2[C@H](C)CN(C(=O)[C@H]3CN3)C[C@@H]2C)c2cc(F)c(-c3c(O)cccc3F)nc21] |
| 136 | 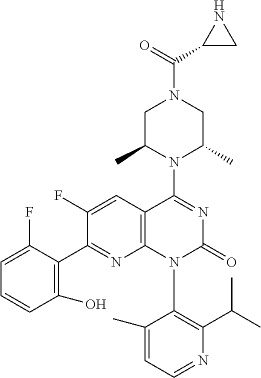 [SMILES: Cc1ccnc(C(C)C)c1-n1c(=O)nc(N2[C@@H](C)CN(C(=O)[C@H]3CN3)C[C@@H]2C)c2cc(F)c(-c3c(O)cccc3F)nc21] |
| 137 | 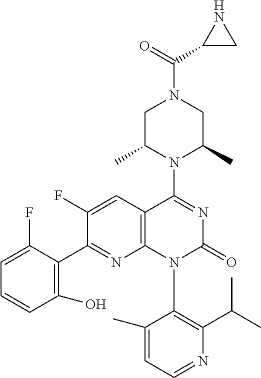 [SMILES: Cc1ccnc(C(C)C)c1-n1c(=O)nc(N2[C@H](C)CN(C(=O)[C@H]3CN3)C[C@H]2C)c2cc(F)c(-c3c(O)cccc3F)nc21] |
| 138 | 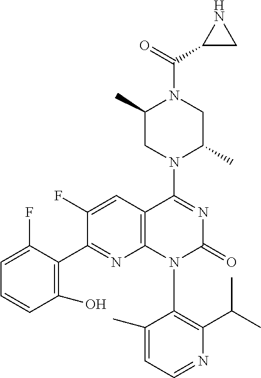 [SMILES: Cc1ccnc(C(C)C)c1-n1c(=O)nc(N2C[C@@H](C)N(C(=O)[C@H]3CN3)C[C@@H]2C)c2cc(F)c(-c3c(O)cccc3F)nc21] |
| 139 | 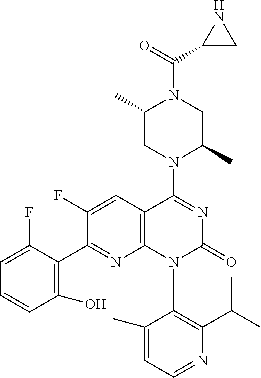 [SMILES: Cc1ccnc(C(C)C)c1-n1c(=O)nc(N2C[C@H](C)N(C(=O)[C@H]3CN3)C[C@H]2C)c2cc(F)c(-c3c(O)cccc3F)nc21] |
| 140 | 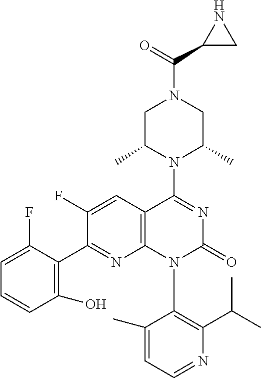 [SMILES: Cc1ccnc(C(C)C)c1-n1c(=O)nc(N2[C@H](C)CN(C(=O)[C@@H]3CN3)C[C@@H]2C)c2cc(F)c(-c3c(O)cccc3F)nc21] |
| 141 | 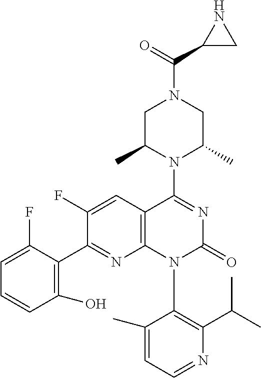 [SMILES: Cc1ccnc(C(C)C)c1-n1c(=O)nc(N2[C@@H](C)CN(C(=O)[C@@H]3CN3)C[C@@H]2C)c2cc(F)c(-c3c(O)cccc3F)nc21] |
| 142 | 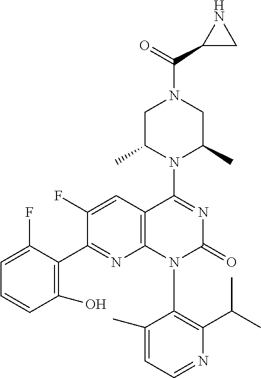 [SMILES: Cc1ccnc(C(C)C)c1-n1c(=O)nc(N2[C@H](C)CN(C(=O)[C@@H]3CN3)C[C@H]2C)c2cc(F)c(-c3c(O)cccc3F)nc21] |
| 143 | 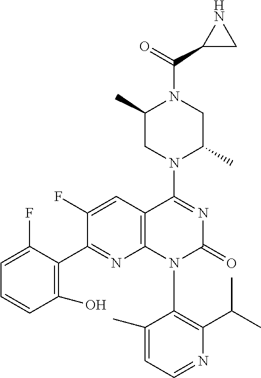 [SMILES: Cc1ccnc(C(C)C)c1-n1c(=O)nc(N2C[C@@H](C)N(C(=O)[C@@H]3CN3)C[C@@H]2C)c2cc(F)c(-c3c(O)cccc3F)nc21] |
| 144 | 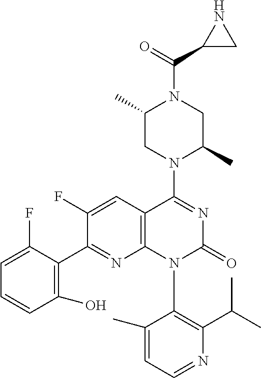 [SMILES: Cc1ccnc(C(C)C)c1-n1c(=O)nc(N2C[C@H](C)N(C(=O)[C@@H]3CN3)C[C@H]2C)c2cc(F)c(-c3c(O)cccc3F)nc21] |
| 145 | 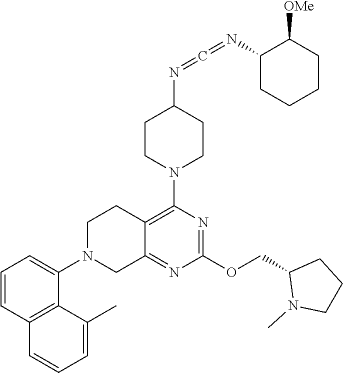 [SMILES: CO[C@H]1CCCC[C@@H]1N=C=NC1CCN(c2nc(OC[C@@H]3CCCN3C)nc3c2CCN(c2cccc4cccc(C)c24)C3)CC1] |
| 146 | 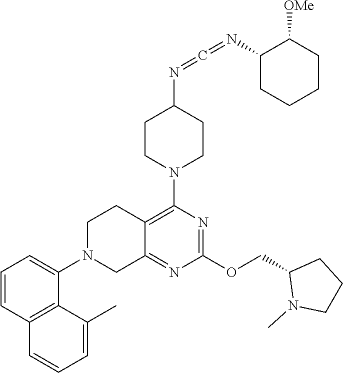 [SMILES: CO[C@@H]1CCCC[C@@H]1N=C=NC1CCN(c2nc(OC[C@@H]3CCCN3C)nc3c2CCN(c2cccc4cccc(C)c24)C3)CC1] |
| 147 | 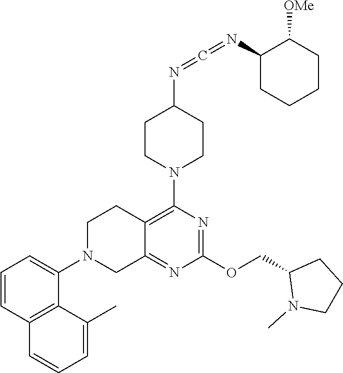 [SMILES: CO[C@@H]1CCCC[C@H]1N=C=NC1CCN(c2nc(OC[C@@H]3CCCN3C)nc3c2CCN(c2cccc4cccc(C)c24)C3)CC1] |
| 148 | 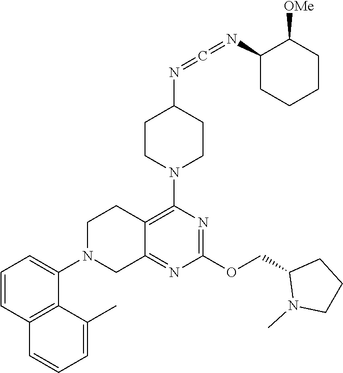 [SMILES: CO[C@H]1CCCC[C@H]1N=C=NC1CCN(c2nc(OC[C@@H]3CCCN3C)nc3c2CCN(c2cccc4cccc(C)c24)C3)CC1] |
| 149 | 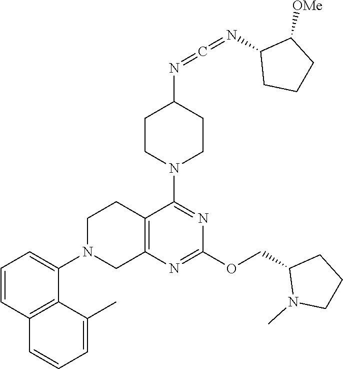 [SMILES: CO[C@@H]1CCC[C@@H]1N=C=NC1CCN(c2nc(OC[C@@H]3CCCN3C)nc3c2CCN(c2cccc4cccc(C)c24)C3)CC1] |
| 150 | 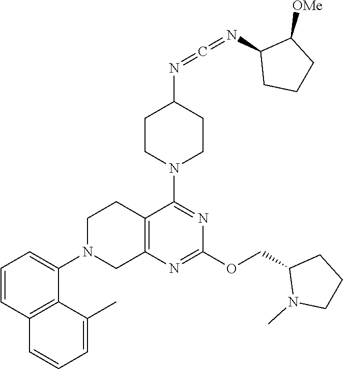 [SMILES: CO[C@H]1CCC[C@H]1N=C=NC1CCN(c2nc(OC[C@@H]3CCCN3C)nc3c2CCN(c2cccc4cccc(C)c24)C3)CC1] |
| 151 | 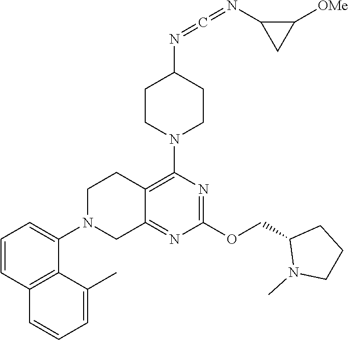 [SMILES: COC1CC1N=C=NC1CCN(c2nc(OC[C@@H]3CCCN3C)nc3c2CCN(c2cccc4cccc(C)c24)C3)CC1] |
| 152 | 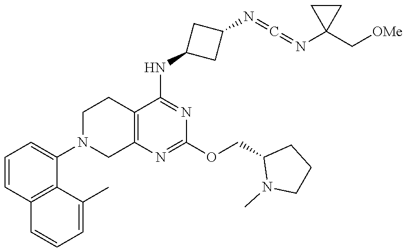 [SMILES: COCC1(N=C=N[C@H]2C[C@H](Nc3nc(OC[C@@H]4CCCN4C)nc4c3CCN(c3cccc5cccc(C)c35)C4)C2)CC1] |
| 153 | 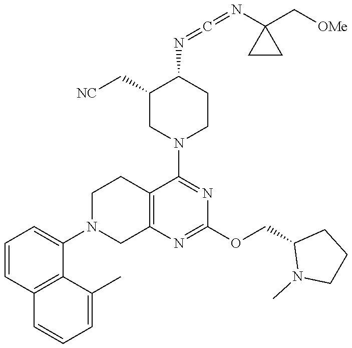 [SMILES: COCC1(N=C=N[C@@H]2CCN(c3nc(OC[C@@H]4CCCN4C)nc4c3CCN(c3cccc5cccc(C)c35)C4)C[C@@H]2CC#N)CC1] |
| 154 | 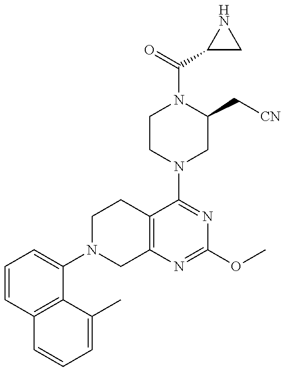 [SMILES: COc1nc2c(c(N3CCN(C(=O)[C@H]4CN4)[C@@H](CC#N)C3)n1)CCN(c1cccc3cccc(C)c13)C2] |
| 155 | 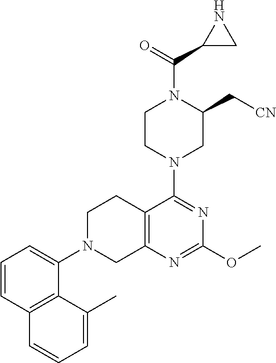 [SMILES: COc1nc2c(c(N3CCN(C(=O)[C@@H]4CN4)[C@@H](CC#N)C3)n1)CCN(c1cccc3cccc(C)c13)C2] |
| 156 | 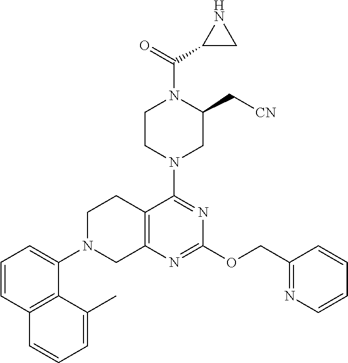 [SMILES: Cc1cccc2cccc(N3CCc4c(nc(OCc5ccccn5)nc4N4CCN(C(=O)[C@H]5CN5)[C@@H](CC#N)C4)C3)c12] |
| 157 | 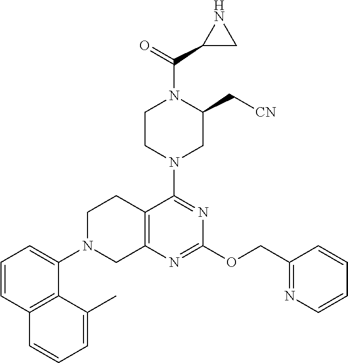 [SMILES: Cc1cccc2cccc(N3CCc4c(nc(OCc5ccccn5)nc4N4CCN(C(=O)[C@@H]5CN5)[C@@H](CC#N)C4)C3)c12] |
| 158 | 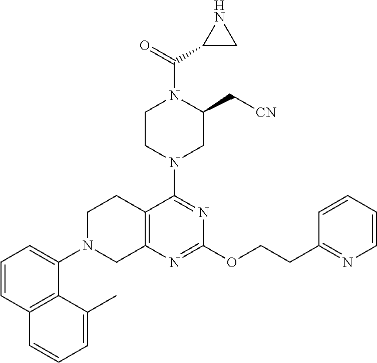 [SMILES: Cc1cccc2cccc(N3CCc4c(nc(OCCc5ccccn5)nc4N4CCN(C(=O)[C@H]5CN5)[C@@H](CC#N)C4)C3)c12] |
| 159 | 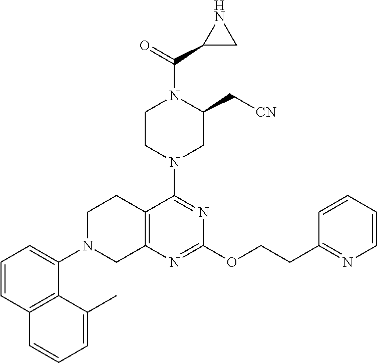 [SMILES: Cc1cccc2cccc(N3CCc4c(nc(OCCc5ccccn5)nc4N4CCN(C(=O)[C@@H]5CN5)[C@@H](CC#N)C4)C3)c12] |
| 160 | 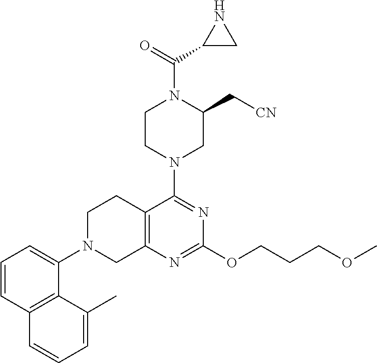 [SMILES: COCCCOc1nc2c(c(N3CCN(C(=O)[C@H]4CN4)[C@@H](CC#N)C3)n1)CCN(c1cccc3cccc(C)c13)C2] |
| 161 | 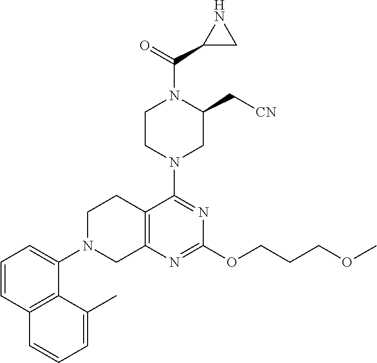 [SMILES: COCCCOc1nc2c(c(N3CCN(C(=O)[C@@H]4CN4)[C@@H](CC#N)C3)n1)CCN(c1cccc3cccc(C)c13)C2] |
| 162 | 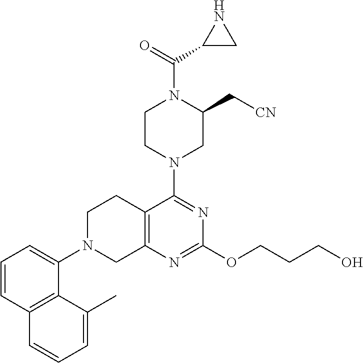 [SMILES: Cc1cccc2cccc(N3CCc4c(nc(OCCCO)nc4N4CCN(C(=O)[C@H]5CN5)[C@@H](CC#N)C4)C3)c12] |
| 163 | 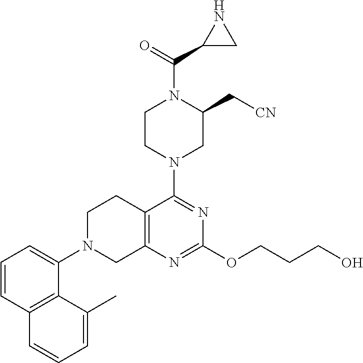 [SMILES: Cc1cccc2cccc(N3CCc4c(nc(OCCCO)nc4N4CCN(C(=O)[C@@H]5CN5)[C@@H](CC#N)C4)C3)c12] |
| 164 | 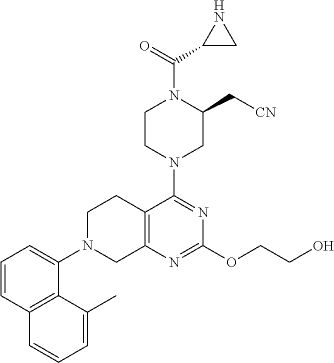 [SMILES: Cc1cccc2cccc(N3CCc4c(nc(OCCO)nc4N4CCN(C(=O)[C@H]5CN5)[C@@H](CC#N)C4)C3)c12] |
| 165 | 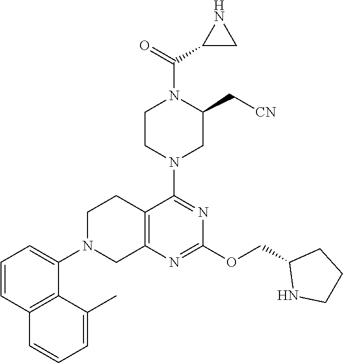 [SMILES: Cc1cccc2cccc(N3CCc4c(nc(OC[C@@H]5CCCN5)nc4N4CCN(C(=O)[C@H]5CN5)[C@@H](CC#N)C4)C3)c12] |
| 166 | 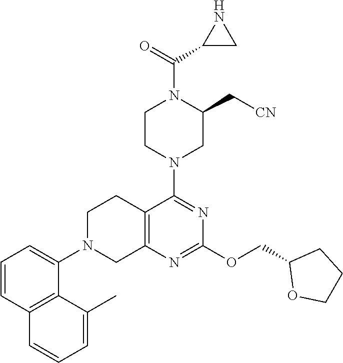 [SMILES: Cc1cccc2cccc(N3CCc4c(nc(OC[C@@H]5CCCO5)nc4N4CCN(C(=O)[C@H]5CN5)[C@@H](CC#N)C4)C3)c12] |
| 167 | 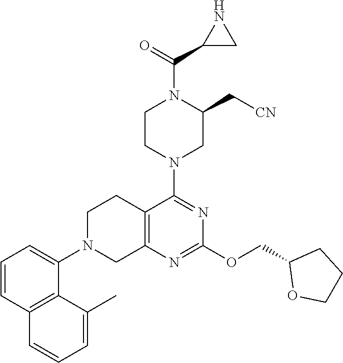 [SMILES: Cc1cccc2cccc(N3CCc4c(nc(OC[C@@H]5CCCO5)nc4N4CCN(C(=O)[C@@H]5CN5)[C@@H](CC#N)C4)C3)c12] |
| 168 | 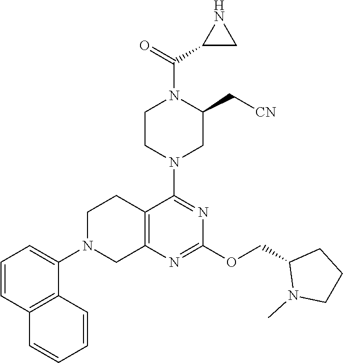 [SMILES: CN1CCC[C@H]1COc1nc2c(c(N3CCN(C(=O)[C@H]4CN4)[C@@H](CC#N)C3)n1)CCN(c1cccc3ccccc13)C2] |
| 169 | 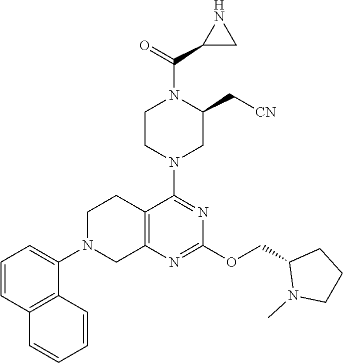 [SMILES: CN1CCC[C@H]1COc1nc2c(c(N3CCN(C(=O)[C@@H]4CN4)[C@@H](CC#N)C3)n1)CCN(c1cccc3ccccc13)C2] |
| 170 | 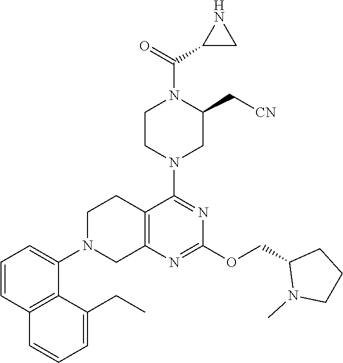 [SMILES: CCc1cccc2cccc(N3CCc4c(nc(OC[C@@H]5CCCN5C)nc4N4CCN(C(=O)[C@H]5CN5)[C@@H](CC#N)C4)C3)c12] |
| 171 | 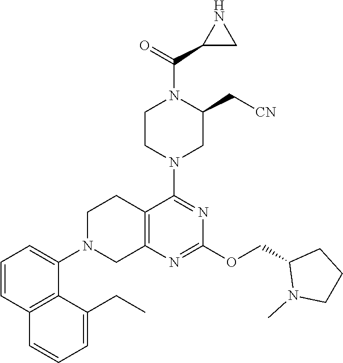 [SMILES: CCc1cccc2cccc(N3CCc4c(nc(OC[C@@H]5CCCN5C)nc4N4CCN(C(=O)[C@@H]5CN5)[C@@H](CC#N)C4)C3)c12] |
| 172 | 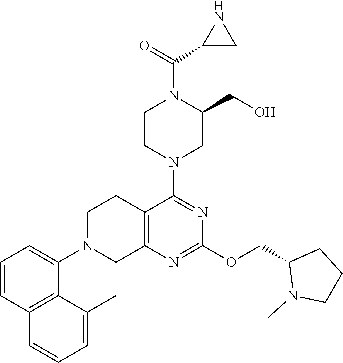 [SMILES: Cc1cccc2cccc(N3CCc4c(nc(OC[C@@H]5CCCN5C)nc4N4CCN(C(=O)[C@H]5CN5)[C@@H](CO)C4)C3)c12] |
| 173 | 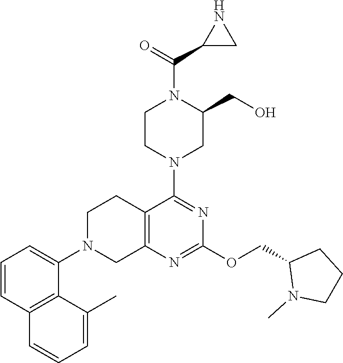 [SMILES: Cc1cccc2cccc(N3CCc4c(nc(OC[C@@H]5CCCN5C)nc4N4CCN(C(=O)[C@@H]5CN5)[C@@H](CO)C4)C3)c12] |
| 174 | 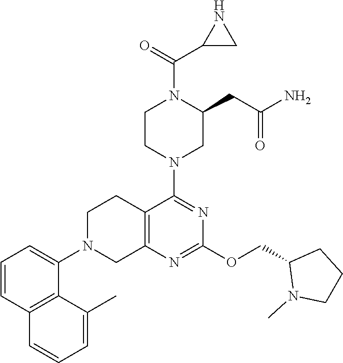 [SMILES: Cc1cccc2cccc(N3CCc4c(nc(OC[C@@H]5CCCN5C)nc4N4CCN(C(=O)C5CN5)[C@@H](CC(N)=O)C4)C3)c12] |
| 175 | 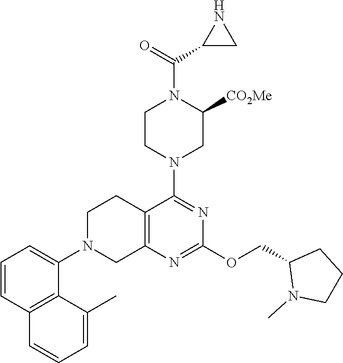 [SMILES: COC(=O)[C@H]1CN(c2nc(OC[C@@H]3CCCN3C)nc3c2CCN(c2cccc4cccc(C)c24)C3)CCN1C(=O)[C@H]1CN1] |
| 176 | 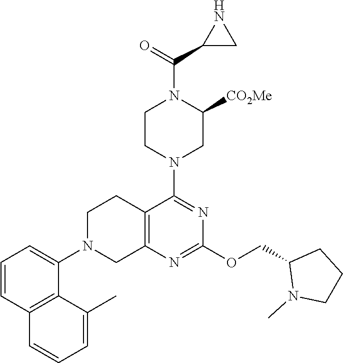 [SMILES: COC(=O)[C@H]1CN(c2nc(OC[C@@H]3CCCN3C)nc3c2CCN(c2cccc4cccc(C)c24)C3)CCN1C(=O)[C@@H]1CN1] |
| 177 | 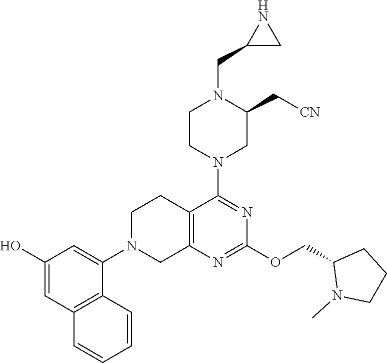 [SMILES: CN1CCC[C@H]1COc1nc2c(c(N3CCN(C[C@@H]4CN4)[C@@H](CC#N)C3)n1)CCN(c1cc(O)cc3ccccc13)C2] |
| 178 | 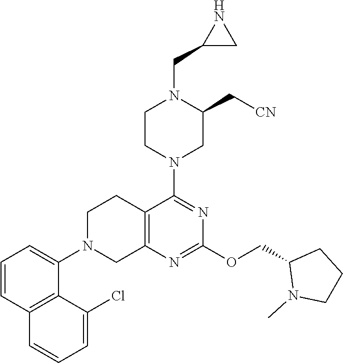 [SMILES: CN1CCC[C@H]1COc1nc2c(c(N3CCN(C[C@@H]4CN4)[C@@H](CC#N)C3)n1)CCN(c1cccc3cccc(Cl)c13)C2] |
| 179 | 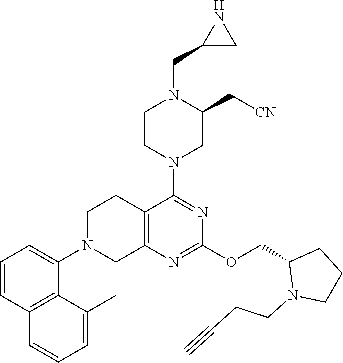 [SMILES: C#CCCN1CCC[C@H]1COc1nc2c(c(N3CCN(C[C@@H]4CN4)[C@@H](CC#N)C3)n1)CCN(c1cccc3cccc(C)c13)C2] |
| 180 | 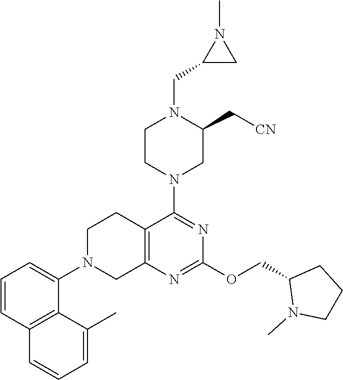 [SMILES: Cc1cccc2cccc(N3CCc4c(nc(OC[C@@H]5CCCN5C)nc4N4CCN(C[C@H]5CN5C)[C@@H](CC#N)C4)C3)c12] |

In some embodiments, a compound of the present invention is selected from Table 2e:

TABLE 2e
Certain Compounds of the Present Invention

| # | Structure |
|---|---|
| 181 | 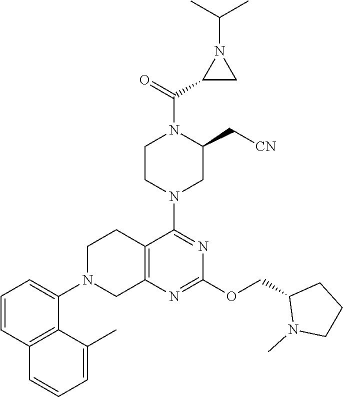 [SMILES: Cc1cccc2cccc(N3CCc4c(nc(OC[C@@H]5CCCN5C)nc4N4CCN(C(=O)[C@H]5CN5C(C)C)[C@@H](CC#N)C4)C3)c12] |
| 182 | 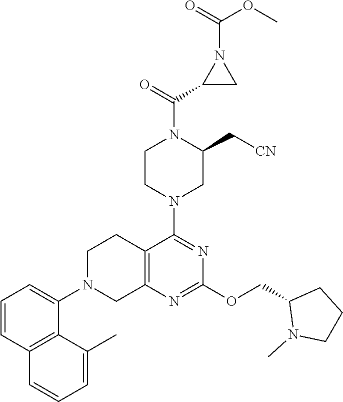 [SMILES: COC(=O)N1C[C@@H]1C(=O)N1CCN(c2nc(OC[C@@H]3CCCN3C)nc3c2CCN(c2cccc4cccc(C)c24)C3)C[C@@H]1CC#N] |
| 183 | 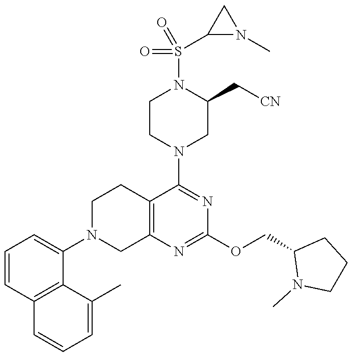 [SMILES: Cc1cccc2cccc(N3CCc4c(nc(OC[C@@H]5CCCN5C)nc4N4CCN(S(=O)(=O)C5CN5C)[C@@H](CC#N)C4)C3)c12] |
| 184 | 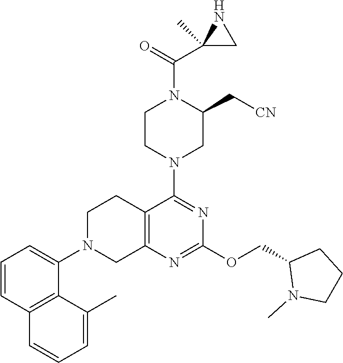 [SMILES: Cc1cccc2cccc(N3CCc4c(nc(OC[C@@H]5CCCN5C)nc4N4CCN(C(=O)[C@]5(C)CN5)[C@@H](CC#N)C4)C3)c12] |
| 185 | 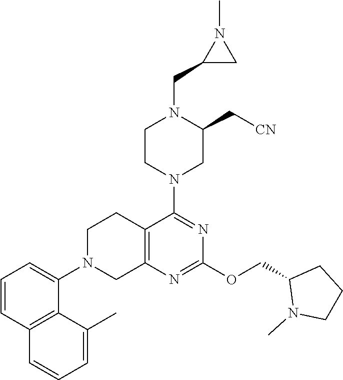 [SMILES: Cc1cccc2cccc(N3CCc4c(nc(OC[C@@H]5CCCN5C)nc4N4CCN(C[C@@H]5CN5C)[C@@H](CC#N)C4)C3)c12] |
| 186 | 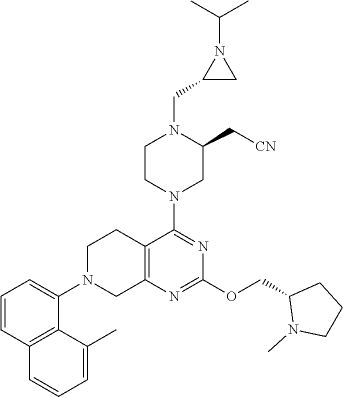 [SMILES: Cc1cccc2cccc(N3CCc4c(nc(OC[C@@H]5CCCN5C)nc4N4CCN(C[C@H]5CN5C(C)C)[C@@H](CC#N)C4)C3)c12] |
| 187 | 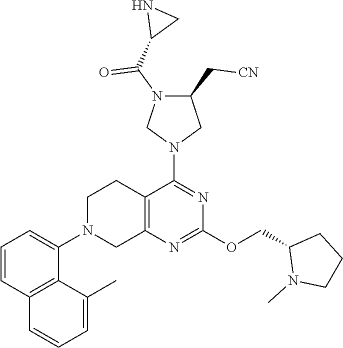 [SMILES: Cc1cccc2cccc(N3CCc4c(nc(OC[C@@H]5CCCN5C)nc4N4C[C@H](CC#N)N(C(=O)[C@H]5CN5)C4)C3)c12] |
| 188 | 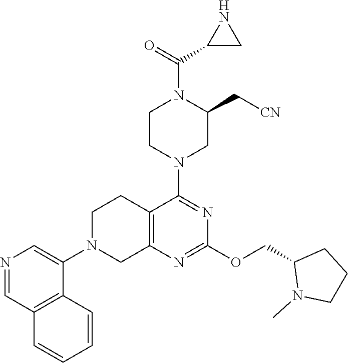 [SMILES: CN1CCC[C@H]1COc1nc2c(c(N3CCN(C(=O)[C@H]4CN4)[C@@H](CC#N)C3)n1)CCN(c1cncc3ccccc13)C2] |
| 189 | 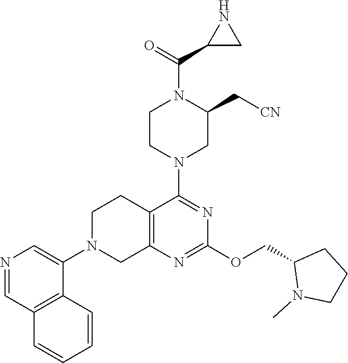 [SMILES: CN1CCC[C@H]1COc1nc2c(c(N3CCN(C(=O)[C@@H]4CN4)[C@@H](CC#N)C3)n1)CCN(c1cncc3ccccc13)C2] |
| 190 | 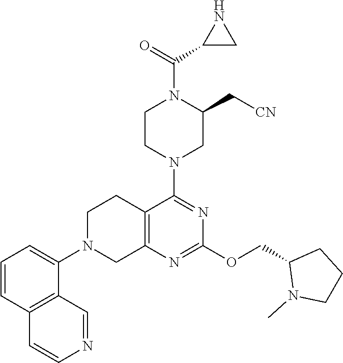 [SMILES: CN1CCC[C@H]1COc1nc2c(c(N3CCN(C(=O)[C@H]4CN4)[C@@H](CC#N)C3)n1)CCN(c1cccc3ccncc13)C2] |
| 191 | 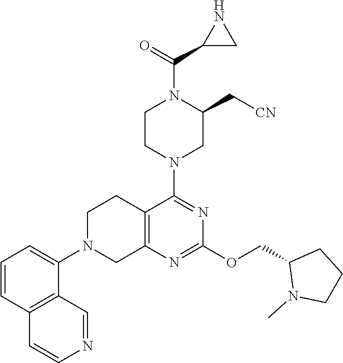 [SMILES: CN1CCC[C@H]1COc1nc2c(c(N3CCN(C(=O)[C@@H]4CN4)[C@@H](CC#N)C3)n1)CCN(c1cccc3ccncc13)C2] |
| 192 | 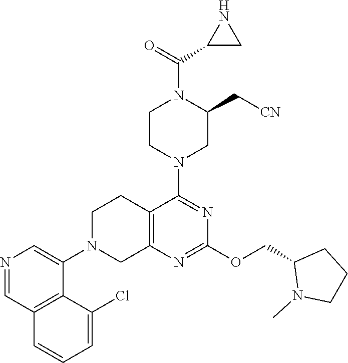 [SMILES: CN1CCC[C@H]1COc1nc2c(c(N3CCN(C(=O)[C@H]4CN4)[C@@H](CC#N)C3)n1)CCN(c1cncc3cccc(Cl)c13)C2] |
| 193 | 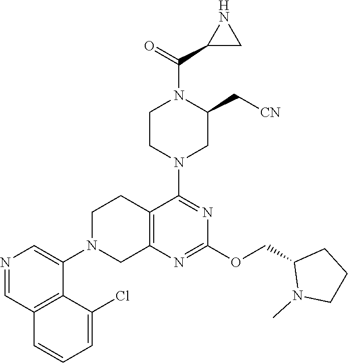 [SMILES: CN1CCC[C@H]1COc1nc2c(c(N3CCN(C(=O)[C@@H]4CN4)[C@@H](CC#N)C3)n1)CCN(c1cncc3cccc(Cl)c13)C2] |
| 194 | 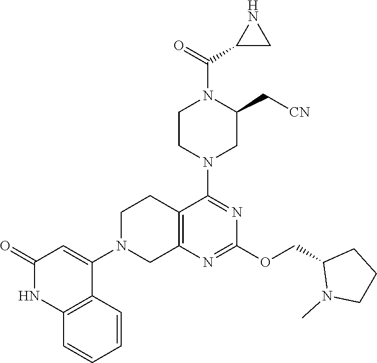 [SMILES: CN1CCC[C@H]1COc1nc2c(c(N3CCN(C(=O)[C@H]4CN4)[C@@H](CC#N)C3)n1)CCN(c1cc(=O)[nH]c3ccccc13)C2] |
| 195 | 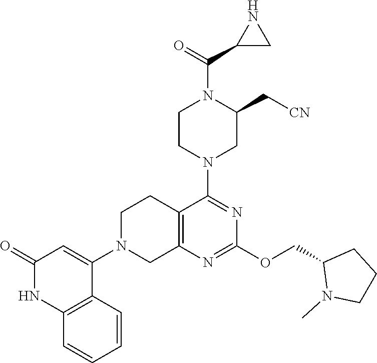 [SMILES: CN1CCC[C@H]1COc1nc2c(c(N3CCN(C(=O)[C@@H]4CN4)[C@@H](CC#N)C3)n1)CCN(c1cc(=O)[nH]c3ccccc13)C2] |
| 196 | 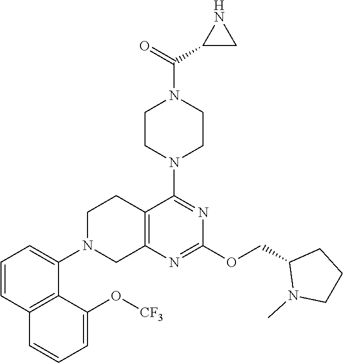 [SMILES: CN1CCC[C@H]1COc1nc2c(c(N3CCN(C(=O)[C@H]4CN4)CC3)n1)CCN(c1cccc3cccc(OC(F)(F)F)c13)C2] |
| 197 | 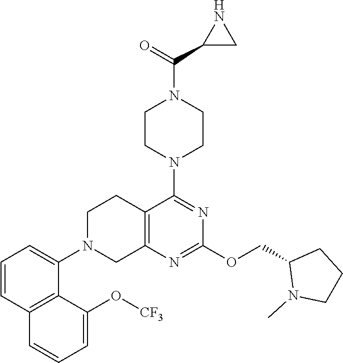 [SMILES: CN1CCC[C@H]1COc1nc2c(c(N3CCN(C(=O)[C@@H]4CN4)CC3)n1)CCN(c1cccc3cccc(OC(F)(F)F)c13)C2] |
| 198 | 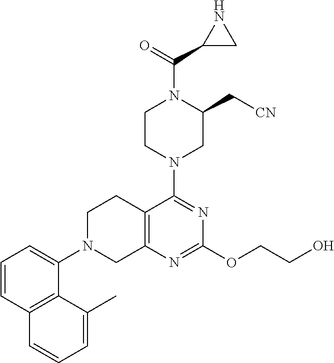 [SMILES: Cc1cccc2cccc(N3CCc4c(nc(OCCO)nc4N4CCN(C(=O)[C@@H]5CN5)[C@@H](CC#N)C4)C3)c12] |
| 199 | 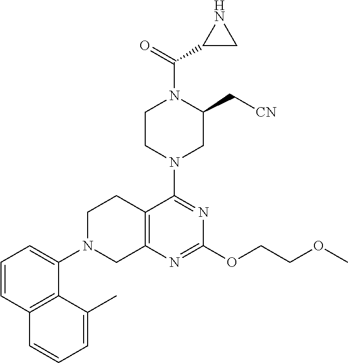 |
| 200 | 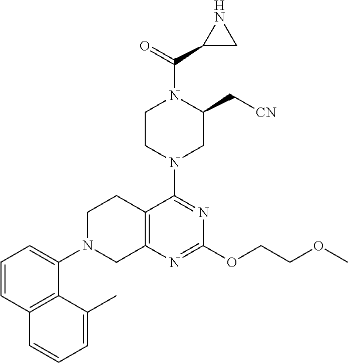 [SMILES: COCCOc1nc2c(c(N3CCN(C(=O)[C@@H]4CN4)[C@@H](CC#N)C3)n1)CCN(c1cccc3cccc(C)c13)C2] |
| 201 | 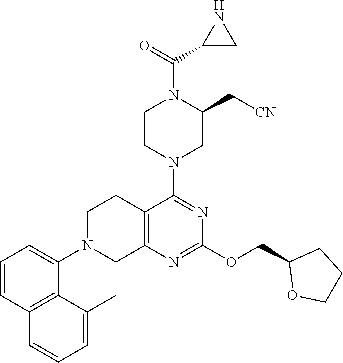 [SMILES: Cc1cccc2cccc(N3CCc4c(nc(OC[C@H]5CCCO5)nc4N4CCN(C(=O)[C@H]5CN5)[C@@H](CC#N)C4)C3)c12] |
| 202 | 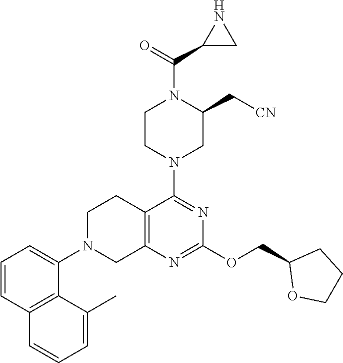 [SMILES: Cc1cccc2cccc(N3CCc4c(nc(OC[C@H]5CCCO5)nc4N4CCN(C(=O)[C@@H]5CN5)[C@@H](CC#N)C4)C3)c12] |
| 203 | 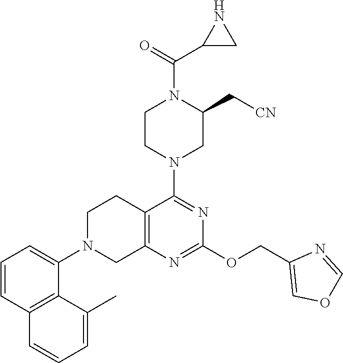 [SMILES: Cc1cccc2cccc(N3CCc4c(nc(OCc5cocn5)nc4N4CCN(C(=O)C5CN5)[C@@H](CC#N)C4)C3)c12] |
| 204 | 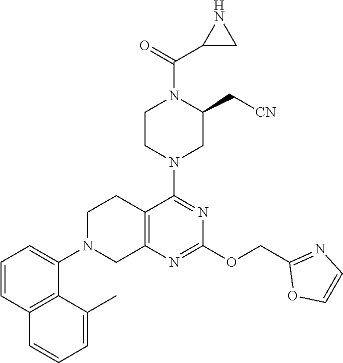 [SMILES: Cc1cccc2cccc(N3CCc4c(nc(OCc5ncco5)nc4N4CCN(C(=O)C5CN5)[C@@H](CC#N)C4)C3)c12] |
| 205 | 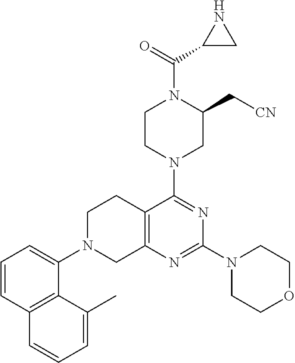 [SMILES: Cc1cccc2cccc(N3CCc4c(nc(N5CCOCC5)nc4N4CCN(C(=O)[C@H]5CN5)[C@@H](CC#N)C4)C3)c12] |
| 206 | 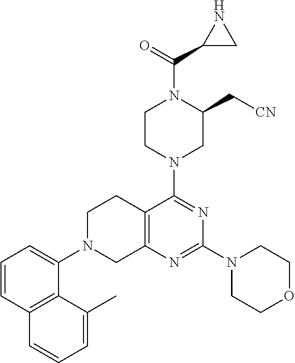 [SMILES: Cc1cccc2cccc(N3CCc4c(nc(N5CCOCC5)nc4N4CCN(C(=O)[C@@H]5CN5)[C@@H](CC#N)C4)C3)c12] |
| 207 | 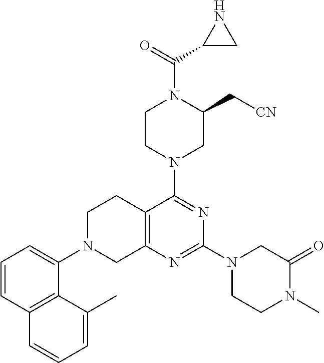 [SMILES: Cc1cccc2cccc(N3CCc4c(nc(N5CCN(C)C(=O)C5)nc4N4CCN(C(=O)[C@H]5CN5)[C@@H](CC#N)C4)C3)c12] |
| 208 | 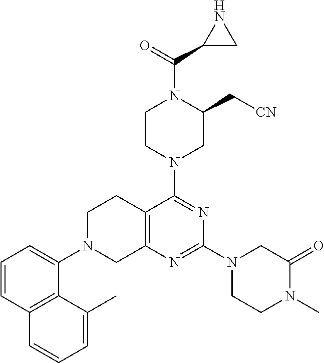 [SMILES: Cc1cccc2cccc(N3CCc4c(nc(N5CCN(C)C(=O)C5)nc4N4CCN(C(=O)[C@@H]5CN5)[C@@H](CC#N)C4)C3)c12] |
| 209 | 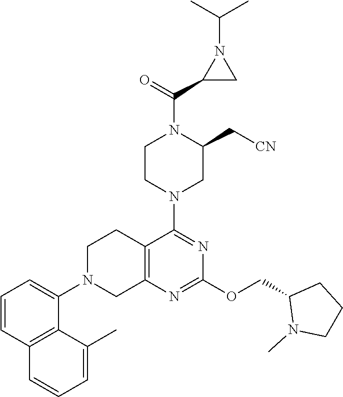 [SMILES: Cc1cccc2cccc(N3CCc4c(nc(OC[C@@H]5CCCN5C)nc4N4CCN(C(=O)[C@@H]5CN5C(C)C)[C@@H](CC#N)C4)C3)c12] |
| 210 | 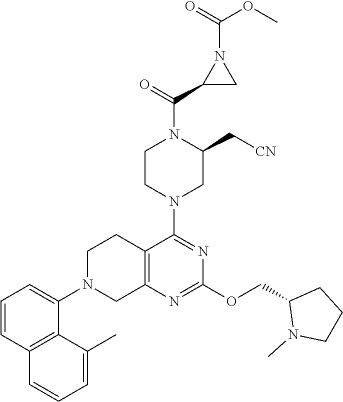 [SMILES: COC(=O)N1C[C@H]1C(=O)N1CCN(c2nc(OC[C@@H]3CCCN3C)nc3c2CCN(c2cccc4cccc(C)c24)C3)C[C@@H]1CC#N] |
| 211 | 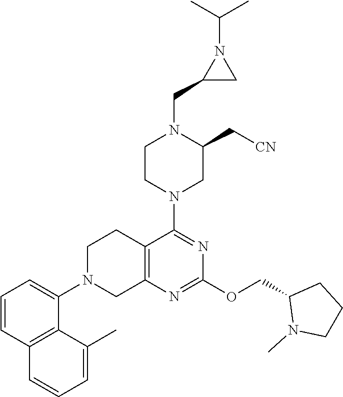 [SMILES: Cc1cccc2cccc(N3CCc4c(nc(OC[C@@H]5CCCN5C)nc4N4CCN(C[C@@H]5CN5C(C)C)[C@@H](CC#N)C4)C3)c12] |
| 212 | 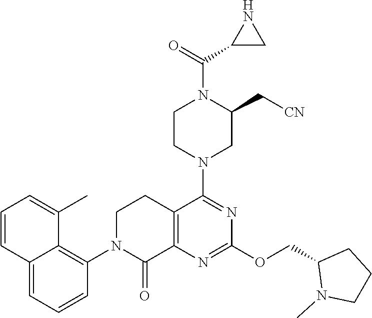 [SMILES: Cc1cccc2cccc(N3CCc4c(nc(OC[C@@H]5CCCN5C)nc4N4CCN(C(=O)[C@H]5CN5)[C@@H](CC#N)C4)C3=O)c12] |
| 213 | 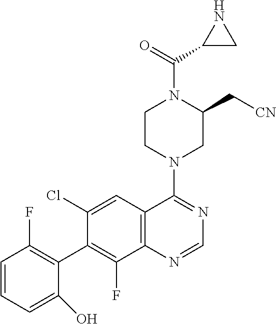 [SMILES: N#CC[C@H]1CN(c2ncnc3c(F)c(-c4c(O)cccc4F)c(Cl)cc23)CCN1C(=O)[C@H]1CN1] |
| 214 | 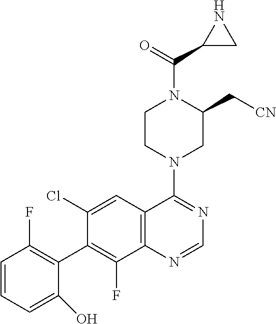 [SMILES: N#CC[C@H]1CN(c2ncnc3c(F)c(-c4c(O)cccc4F)c(Cl)cc23)CCN1C(=O)[C@@H]1CN1] |
| 215 | 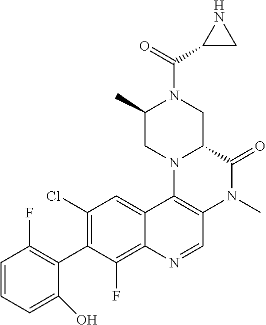 [SMILES: C[C@@H]1CN2c3c(cnc4c(F)c(-c5c(O)cccc5F)c(Cl)cc34)N(C)C(=O)[C@H]2CN1C(=O)[C@H]1CN1] |
| 216 | 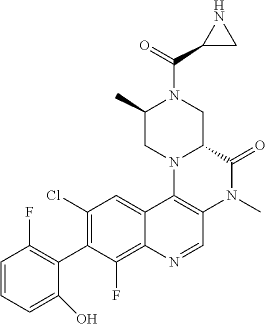 [SMILES: C[C@@H]1CN2c3c(cnc4c(F)c(-c5c(O)cccc5F)c(Cl)cc34)N(C)C(=O)[C@H]2CN1C(=O)[C@@H]1CN1] |

In some embodiments, a compound of the present invention is selected from Table 2f:

TABLE 2f
Certain Compounds of the Present Invention

| # | Structure |
|---|---|
| 217 | 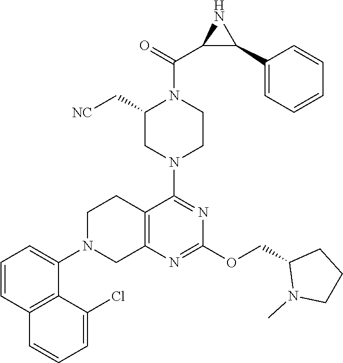 [SMILES: CN1CCC[C@H]1COc1nc2c(c(N3CCN(C(=O)[C@H]4N[C@H]4c4ccccc4)[C@@H](CC#N)C3)n1)CCN(c1cccc3cccc(Cl)c13)C2] |
| 218 | 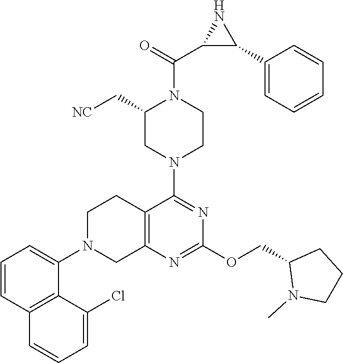 [SMILES: CN1CCC[C@H]1COc1nc2c(c(N3CCN(C(=O)[C@@H]4N[C@@H]4c4ccccc4)[C@@H](CC#N)C3)n1)CCN(c1cccc3cccc(Cl)c13)C2] |
| 219 | 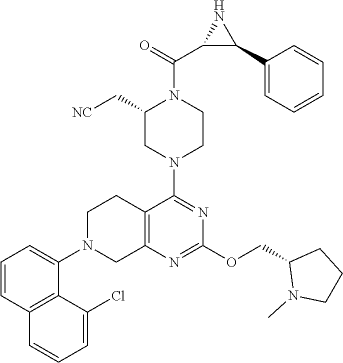 [SMILES: CN1CCC[C@H]1COc1nc2c(c(N3CCN(C(=O)[C@@H]4N[C@H]4c4ccccc4)[C@@H](CC#N)C3)n1)CCN(c1cccc3cccc(Cl)c13)C2] |
| 220 | 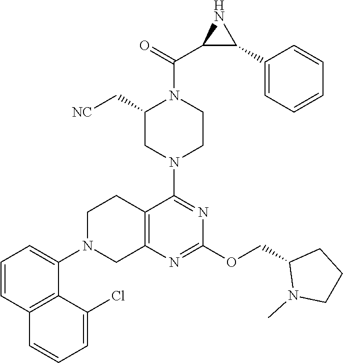 [SMILES: CN1CCC[C@H]1COc1nc2c(c(N3CCN(C(=O)[C@H]4N[C@@H]4c4ccccc4)[C@@H](CC#N)C3)n1)CCN(c1cccc3cccc(Cl)c13)C2] |
| 221 | 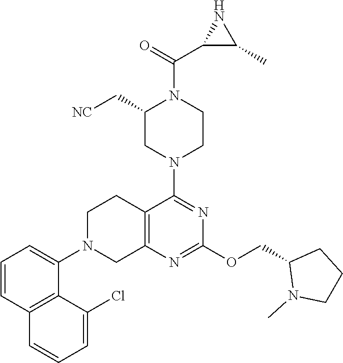 [SMILES: C[C@H]1N[C@H]1C(=O)N1CCN(c2nc(OC[C@@H]3CCCN3C)nc3c2CCN(c2cccc4cccc(Cl)c24)C3)C[C@@H]1CC#N] |
| 222 | 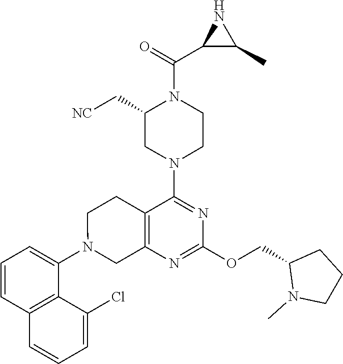 [SMILES: C[C@@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 223 | 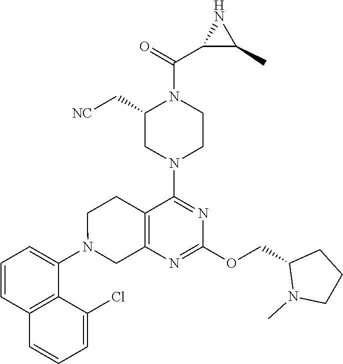 [SMILES: C[C@@H]1N[C@H]1C(=O)N1CCN(c2nc(OC[C@@H]3CCCN3C)nc3c2CCN(c2cccc4cccc(Cl)c24)C3)C[C@@H]1CC#N] |
| 224 | 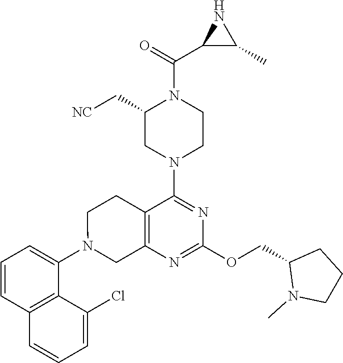 [SMILES: C[C@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 225 | 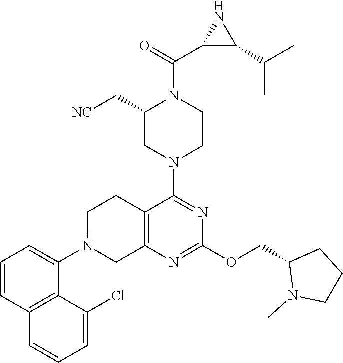 [SMILES: CC(C)[C@H]1N[C@H]1C(=O)N1CCN(c2nc(OC[C@@H]3CCCN3C)nc3c2CCN(c2cccc4cccc(Cl)c24)C3)C[C@@H]1CC#N] |
| 226 | 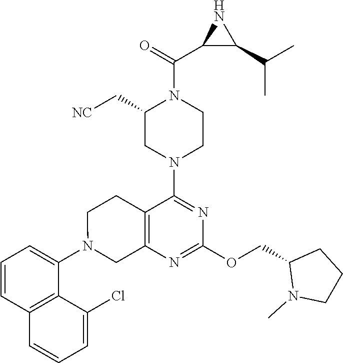 [SMILES: CC(C)[C@@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 227 | 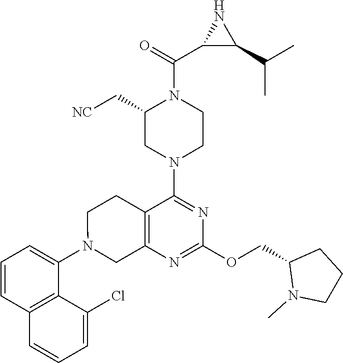 [SMILES: CC(C)[C@@H]1N[C@H]1C(=O)N1CCN(c2nc(OC[C@@H]3CCCN3C)nc3c2CCN(c2cccc4cccc(Cl)c24)C3)C[C@@H]1CC#N] |
| 228 | 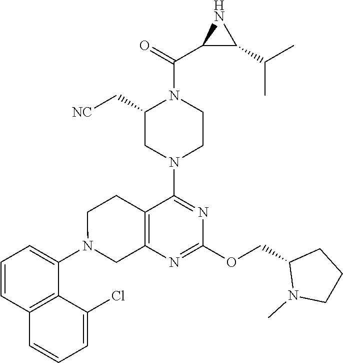 [SMILES: CC(C)[C@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 229 | 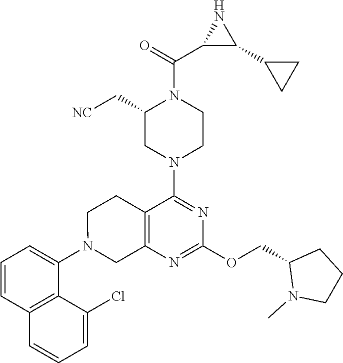 [SMILES: CN1CCC[C@H]1COc1nc2c(c(N3CCN(C(=O)[C@@H]4N[C@@H]4C4CC4)[C@@H](CC#N)C3)n1)CCN(c1cccc3cccc(Cl)c13)C2] |
| 230 | 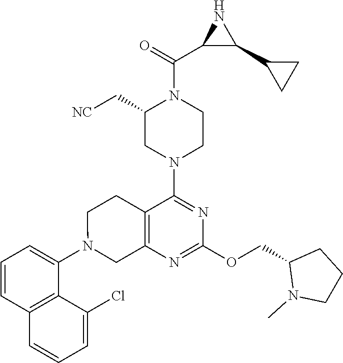 [SMILES: CN1CCC[C@H]1COc1nc2c(c(N3CCN(C(=O)[C@H]4N[C@H]4C4CC4)[C@@H](CC#N)C3)n1)CCN(c1cccc3cccc(Cl)c13)C2] |
| 231 | 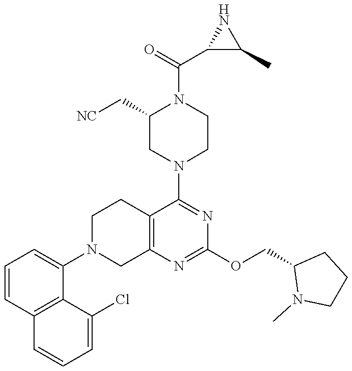 [SMILES: C[C@@H]1N[C@H]1C(=O)N1CCN(c2nc(OC[C@@H]3CCCN3C)nc3c2CCN(c2cccc4cccc(Cl)c24)C3)C[C@@H]1CC#N] |
| 232 | 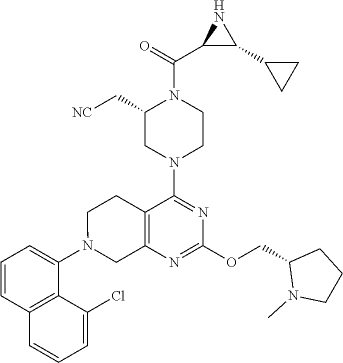 [SMILES: CN1CCC[C@H]1COc1nc2c(c(N3CCN(C(=O)[C@H]4N[C@@H]4C4CC4)[C@@H](CC#N)C3)n1)CCN(c1cccc3cccc(Cl)c13)C2] |
| 233 | 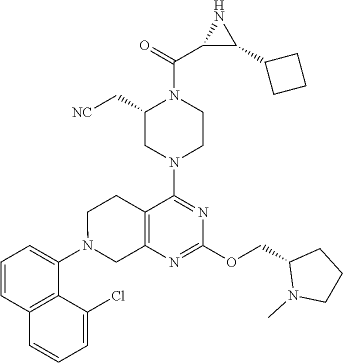 [SMILES: CN1CCC[C@H]1COc1nc2c(c(N3CCN(C(=O)[C@@H]4N[C@@H]4C4CCC4)[C@@H](CC#N)C3)n1)CCN(c1cccc3cccc(Cl)c13)C2] |
| 234 | 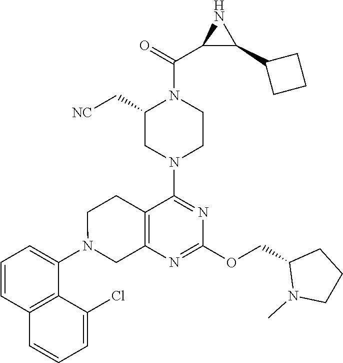 [SMILES: CN1CCC[C@H]1COc1nc2c(c(N3CCN(C(=O)[C@H]4N[C@H]4C4CCC4)[C@@H](CC#N)C3)n1)CCN(c1cccc3cccc(Cl)c13)C2] |
| 235 | 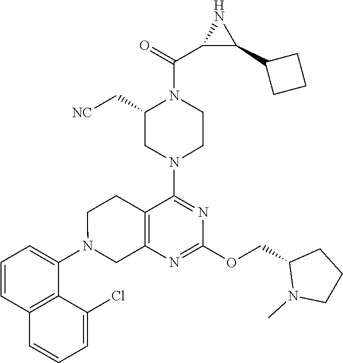 [SMILES: CN1CCC[C@H]1COc1nc2c(c(N3CCN(C(=O)[C@@H]4N[C@H]4C4CCC4)[C@@H](CC#N)C3)n1)CCN(c1cccc3cccc(Cl)c13)C2] |
| 236 | 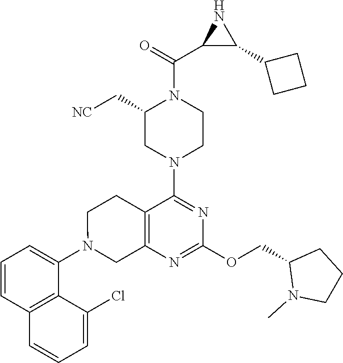 [SMILES: CN1CCC[C@H]1COc1nc2c(c(N3CCN(C(=O)[C@H]4N[C@@H]4C4CCC4)[C@@H](CC#N)C3)n1)CCN(c1cccc3cccc(Cl)c13)C2] |
| 237 | 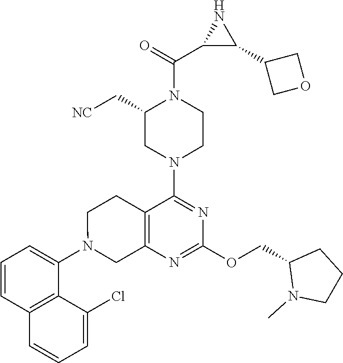 [SMILES: CN1CCC[C@H]1COc1nc2c(c(N3CCN(C(=O)[C@@H]4N[C@@H]4C4COC4)[C@@H](CC#N)C3)n1)CCN(c1cccc3cccc(Cl)c13)C2] |
| 238 | 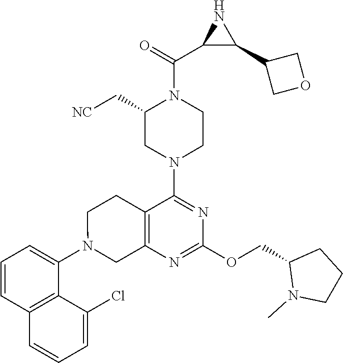 [SMILES: CN1CCC[C@H]1COc1nc2c(c(N3CCN(C(=O)[C@H]4N[C@H]4C4COC4)[C@@H](CC#N)C3)n1)CCN(c1cccc3cccc(Cl)c13)C2] |
| 239 | 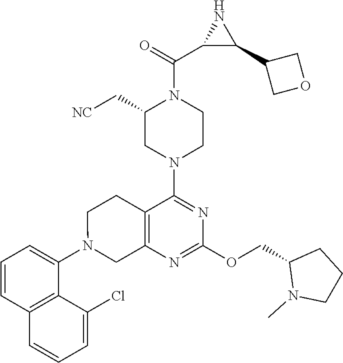 [SMILES: CN1CCC[C@H]1COc1nc2c(c(N3CCN(C(=O)[C@@H]4N[C@H]4C4COC4)[C@@H](CC#N)C3)n1)CCN(c1cccc3cccc(Cl)c13)C2] |
| 240 | 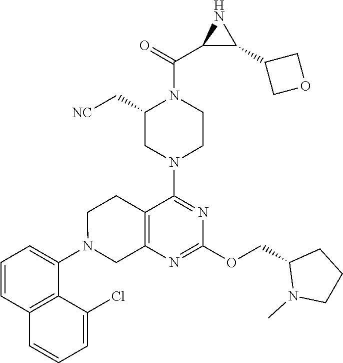 [SMILES: CN1CCC[C@H]1COc1nc2c(c(N3CCN(C(=O)[C@H]4N[C@@H]4C4COC4)[C@@H](CC#N)C3)n1)CCN(c1cccc3cccc(Cl)c13)C2] |
| 241 | 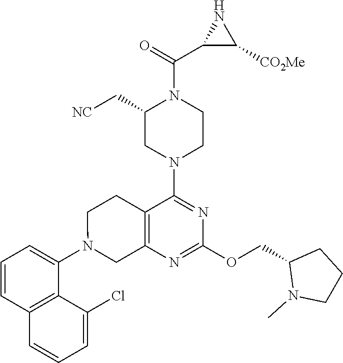 [SMILES: COC(=O)[C@H]1N[C@H]1C(=O)N1CCN(c2nc(OC[C@@H]3CCCN3C)nc3c2CCN(c2cccc4cccc(Cl)c24)C3)C[C@@H]1CC#N] |
| 242 | 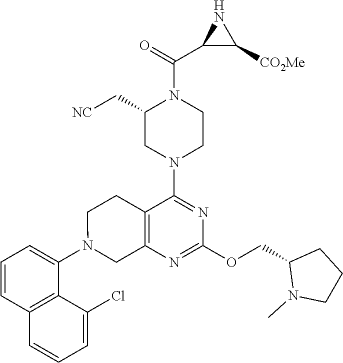 [SMILES: COC(=O)[C@@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 243 | 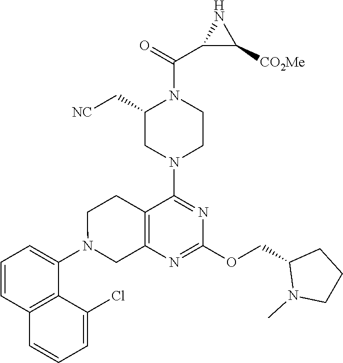 [SMILES: COC(=O)[C@@H]1N[C@H]1C(=O)N1CCN(c2nc(OC[C@@H]3CCCN3C)nc3c2CCN(c2cccc4cccc(Cl)c24)C3)C[C@@H]1CC#N] |
| 244 | 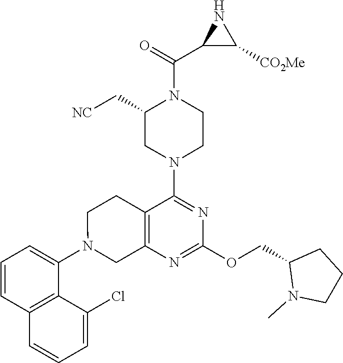 [SMILES: COC(=O)[C@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 245 | 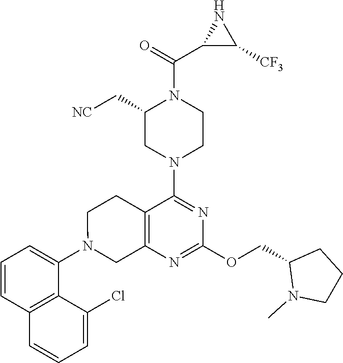 [SMILES: CN1CCC[C@H]1COc1nc2c(c(N3CCN(C(=O)[C@@H]4N[C@@H]4C(F)(F)F)[C@@H](CC#N)C3)n1)CCN(c1cccc3cccc(Cl)c13)C2] |
| 246 | 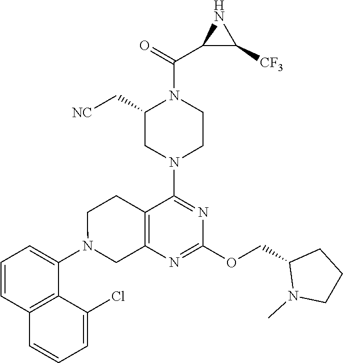 [SMILES: CN1CCC[C@H]1COc1nc2c(c(N3CCN(C(=O)[C@H]4N[C@H]4C(F)(F)F)[C@@H](CC#N)C3)n1)CCN(c1cccc3cccc(Cl)c13)C2] |
| 247 | 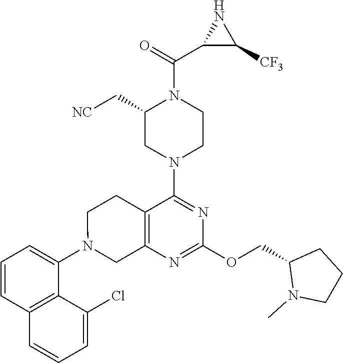 [SMILES: CN1CCC[C@H]1COc1nc2c(c(N3CCN(C(=O)[C@@H]4N[C@H]4C(F)(F)F)[C@@H](CC#N)C3)n1)CCN(c1cccc3cccc(Cl)c13)C2] |
| 248 | 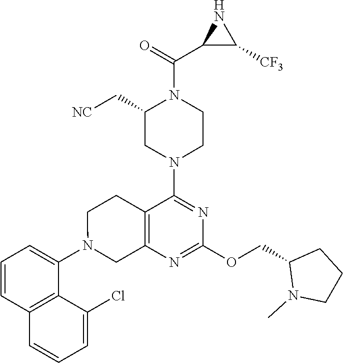 [SMILES: CN1CCC[C@H]1COc1nc2c(c(N3CCN(C(=O)[C@H]4N[C@@H]4C(F)(F)F)[C@@H](CC#N)C3)n1)CCN(c1cccc3cccc(Cl)c13)C2] |
| 249 | 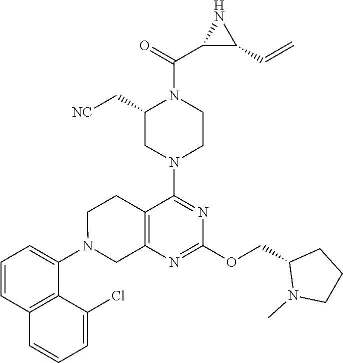 [SMILES: C=C[C@H]1N[C@H]1C(=O)N1CCN(c2nc(OC[C@@H]3CCCN3C)nc3c2CCN(c2cccc4cccc(Cl)c24)C3)C[C@@H]1CC#N] |
| 250 | 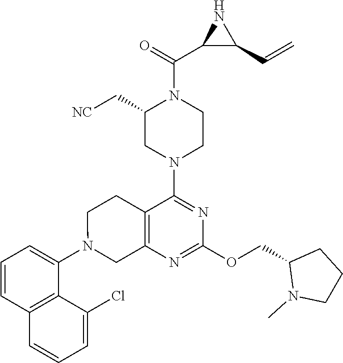 [SMILES: C=C[C@@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 251 | 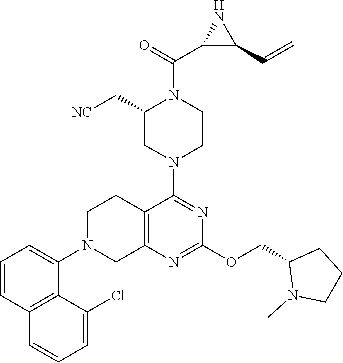 [SMILES: C=C[C@@H]1N[C@H]1C(=O)N1CCN(c2nc(OC[C@@H]3CCCN3C)nc3c2CCN(c2cccc4cccc(Cl)c24)C3)C[C@@H]1CC#N] |
| 252 | 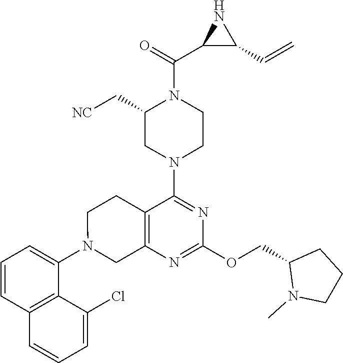 [SMILES: C=C[C@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 253 | 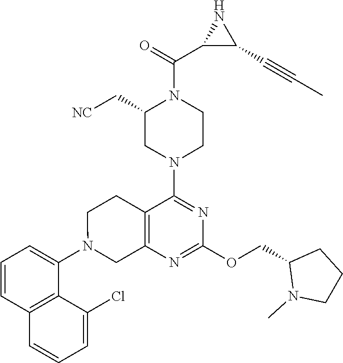 [SMILES: CC#C[C@H]1N[C@H]1C(=O)N1CCN(c2nc(OC[C@@H]3CCCN3C)nc3c2CCN(c2cccc4cccc(Cl)c24)C3)C[C@@H]1CC#N] |
| 254 | 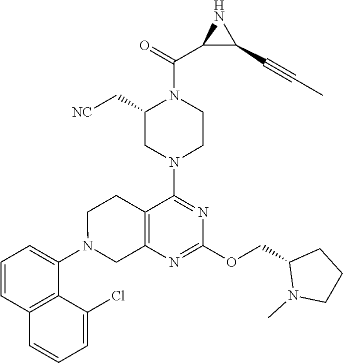 [SMILES: CC#C[C@@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 255 | 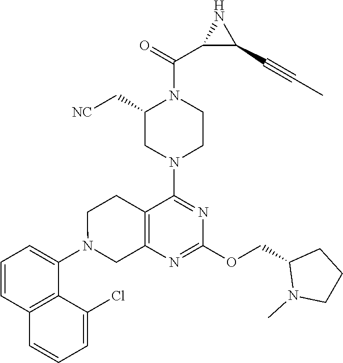 [SMILES: CC#C[C@@H]1N[C@H]1C(=O)N1CCN(c2nc(OC[C@@H]3CCCN3C)nc3c2CCN(c2cccc4cccc(Cl)c24)C3)C[C@@H]1CC#N] |
| 256 | 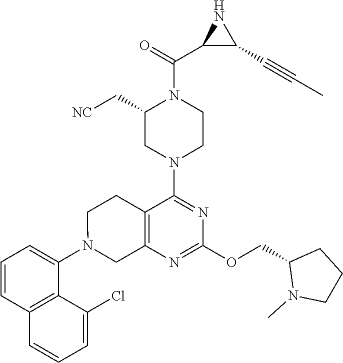 [SMILES: CC#C[C@H]1N[C@@H]1C(=O)N1CCN(c2nc(OC[C@@H]3CCCN3C)nc3c2CCN(c2cccc4cccc(Cl)c24)C3)C[C@@H]1CC#N] |
| 257 | 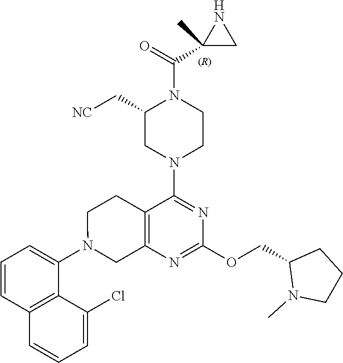 |
| 258 | 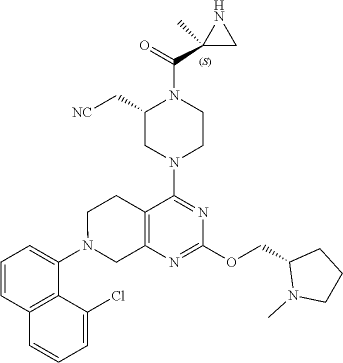 |
| 259 | 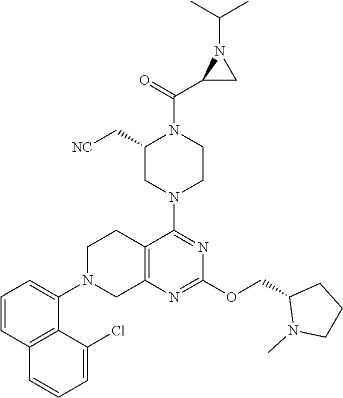 |
| 260 | 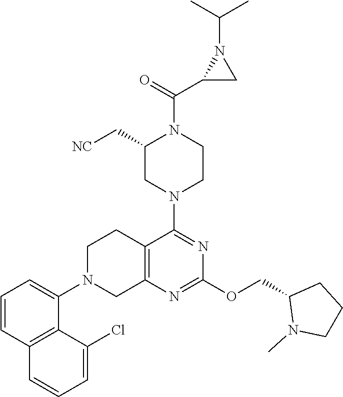 |
| 261 | 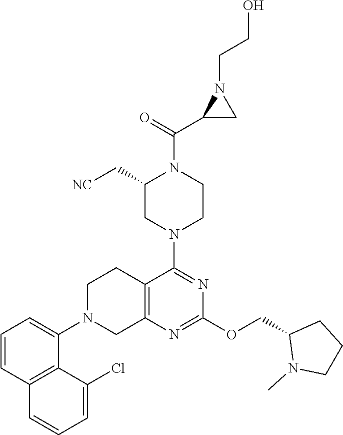 |
| 262 | 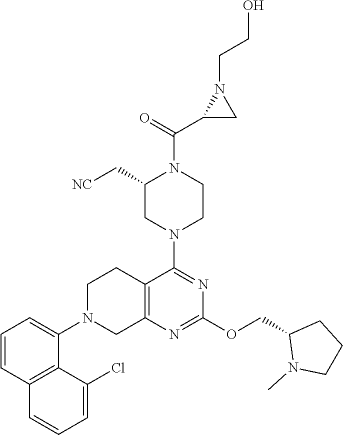 |
| 263 | 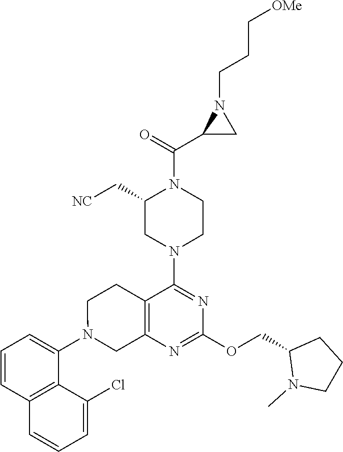 |
| 264 | 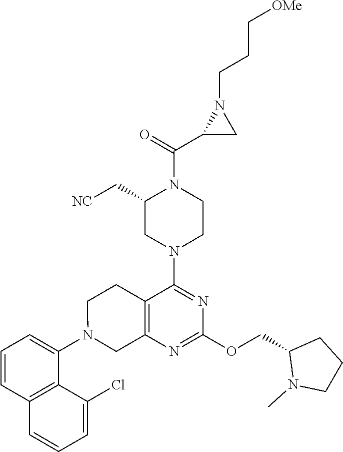 |
| 265 | 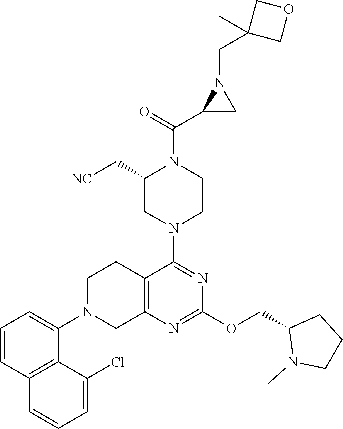 |
| 266 | 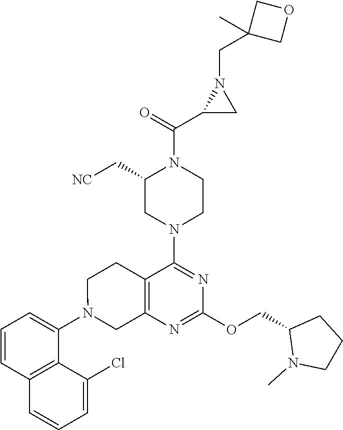 |
| 267 | 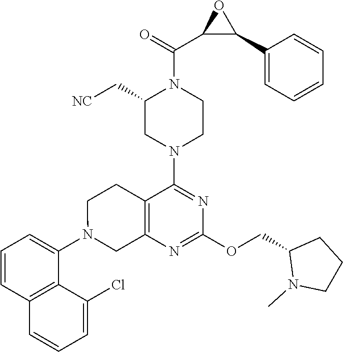 [SMILES: CN1CCC[C@H]1COc1nc2c(c(N3CCN(C(=O)[C@H]4O[C@H]4c4ccccc4)[C@@H](CC#N)C3)n1)CCN(c1cccc3cccc(Cl)c13)C2] |
| 268 | 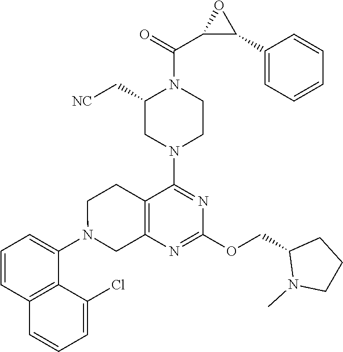 [SMILES: CN1CCC[C@H]1COc1nc2c(c(N3CCN(C(=O)[C@@H]4O[C@@H]4c4ccccc4)[C@@H](CC#N)C3)n1)CCN(c1cccc3cccc(Cl)c13)C2] |
| 269 | 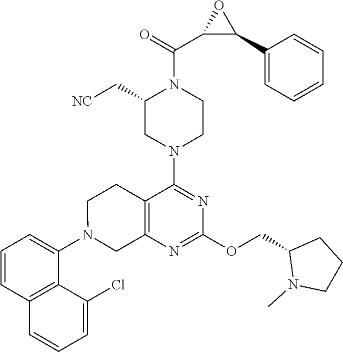 [SMILES: CN1CCC[C@H]1COc1nc2c(c(N3CCN(C(=O)[C@@H]4O[C@H]4c4ccccc4)[C@@H](CC#N)C3)n1)CCN(c1cccc3cccc(Cl)c13)C2] |
| 270 | 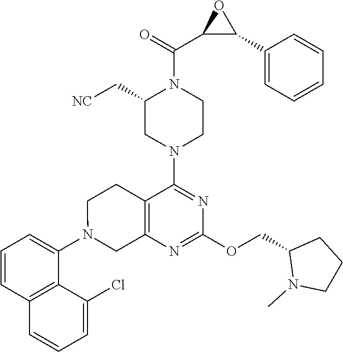 [SMILES: CN1CCC[C@H]1COc1nc2c(c(N3CCN(C(=O)[C@H]4O[C@@H]4c4ccccc4)[C@@H](CC#N)C3)n1)CCN(c1cccc3cccc(Cl)c13)C2] |
| 271 | 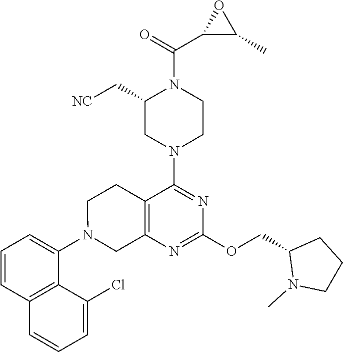 [SMILES: C[C@H]1O[C@H]1C(=O)N1CCN(c2nc(OC[C@@H]3CCCN3C)nc3c2CCN(c2cccc4cccc(Cl)c24)C3)C[C@@H]1CC#N] |
| 272 | 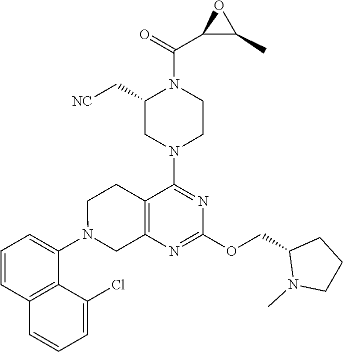 [SMILES: C[C@@H]1O[C@@H]1C(=O)N1CCN(c2nc(OC[C@@H]3CCCN3C)nc3c2CCN(c2cccc4cccc(Cl)c24)C3)C[C@@H]1CC#N] |
| 273 | 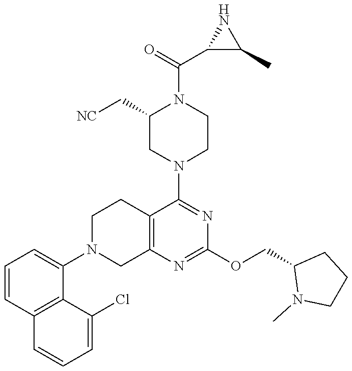 [SMILES: C[C@@H]1N[C@H]1C(=O)N1CCN(c2nc(OC[C@@H]3CCCN3C)nc3c2CCN(c2cccc4cccc(Cl)c24)C3)C[C@@H]1CC#N] |
| 274 | 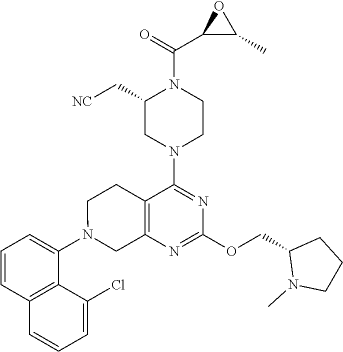 [SMILES: C[C@H]1O[C@@H]1C(=O)N1CCN(c2nc(OC[C@@H]3CCCN3C)nc3c2CCN(c2cccc4cccc(Cl)c24)C3)C[C@@H]1CC#N] |
| 275 | 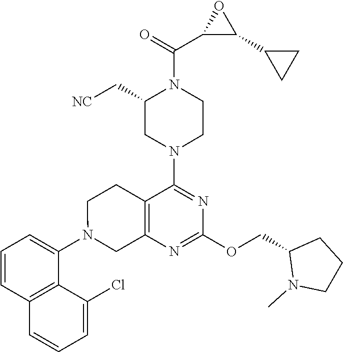 [SMILES: CN1CCC[C@H]1COc1nc2c(c(N3CCN(C(=O)[C@@H]4O[C@@H]4C4CC4)[C@@H](CC#N)C3)n1)CCN(c1cccc3cccc(Cl)c13)C2] |
| 276 | 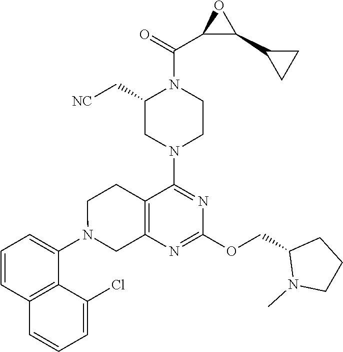 [SMILES: CN1CCC[C@H]1COc1nc2c(c(N3CCN(C(=O)[C@H]4O[C@H]4C4CC4)[C@@H](CC#N)C3)n1)CCN(c1cccc3cccc(Cl)c13)C2] |
| 277 | 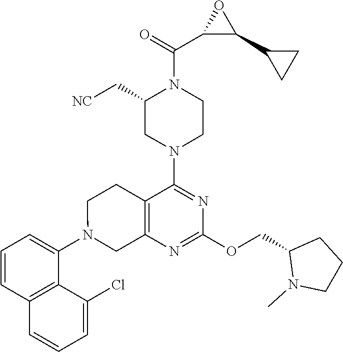 [SMILES: CN1CCC[C@H]1COc1nc2c(c(N3CCN(C(=O)[C@@H]4O[C@H]4C4CC4)[C@@H](CC#N)C3)n1)CCN(c1cccc3cccc(Cl)c13)C2] |
| 278 | 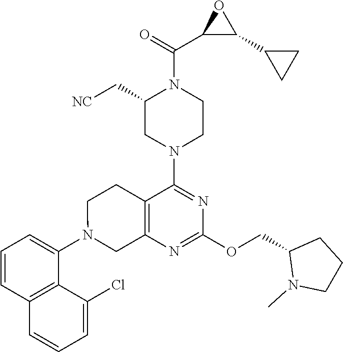 [SMILES: CN1CCC[C@H]1COc1nc2c(c(N3CCN(C(=O)[C@H]4O[C@@H]4C4CC4)[C@@H](CC#N)C3)n1)CCN(c1cccc3cccc(Cl)c13)C2] |
| 279 | 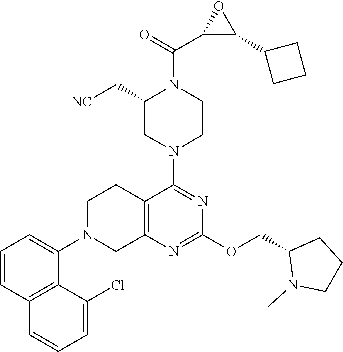 [SMILES: CN1CCC[C@H]1COc1nc2c(c(N3CCN(C(=O)[C@@H]4O[C@@H]4C4CCC4)[C@@H](CC#N)C3)n1)CCN(c1cccc3cccc(Cl)c13)C2] |
| 280 | 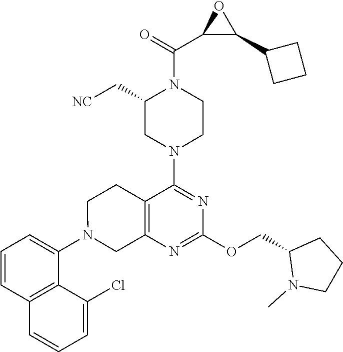 [SMILES: CN1CCC[C@H]1COc1nc2c(c(N3CCN(C(=O)[C@H]4O[C@H]4C4CCC4)[C@@H](CC#N)C3)n1)CCN(c1cccc3cccc(Cl)c13)C2] |
| 281 | 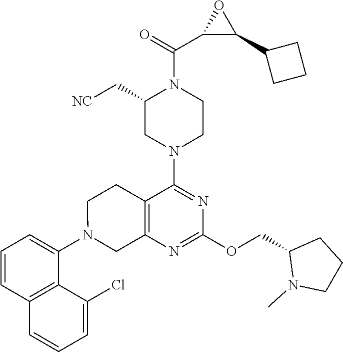 [SMILES: CN1CCC[C@H]1COc1nc2c(c(N3CCN(C(=O)[C@@H]4O[C@H]4C4CCC4)[C@@H](CC#N)C3)n1)CCN(c1cccc3cccc(Cl)c13)C2] |
| 282 | 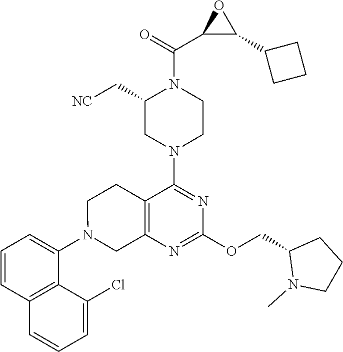 [SMILES: CN1CCC[C@H]1COc1nc2c(c(N3CCN(C(=O)[C@H]4O[C@@H]4C4CCC4)[C@@H](CC#N)C3)n1)CCN(c1cccc3cccc(Cl)c13)C2] |
| 283 | 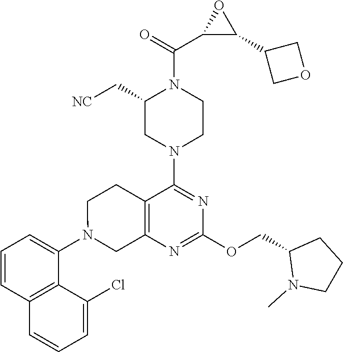 [SMILES: CN1CCC[C@H]1COc1nc2c(c(N3CCN(C(=O)[C@@H]4O[C@@H]4C4COC4)[C@@H](CC#N)C3)n1)CCN(c1cccc3cccc(Cl)c13)C2] |
| 284 | 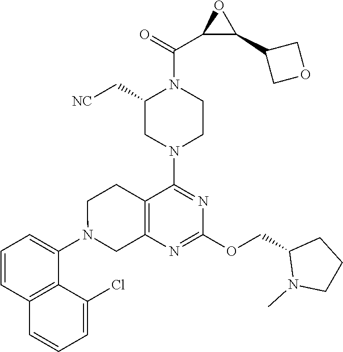 [SMILES: CN1CCC[C@H]1COc1nc2c(c(N3CCN(C(=O)[C@H]4O[C@H]4C4COC4)[C@@H](CC#N)C3)n1)CCN(c1cccc3cccc(Cl)c13)C2] |
| 285 | 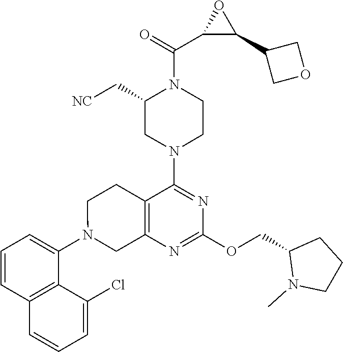 [SMILES: CN1CCC[C@H]1COc1nc2c(c(N3CCN(C(=O)[C@@H]4O[C@H]4C4COC4)[C@@H](CC#N)C3)n1)CCN(c1cccc3cccc(Cl)c13)C2] |
| 286 | 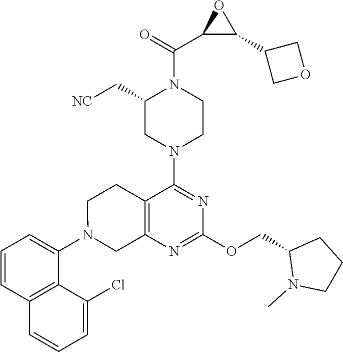 [SMILES: CN1CCC[C@H]1COc1nc2c(c(N3CCN(C(=O)[C@H]4O[C@@H]4C4COC4)[C@@H](CC#N)C3)n1)CCN(c1cccc3cccc(Cl)c13)C2] |
| 287 | 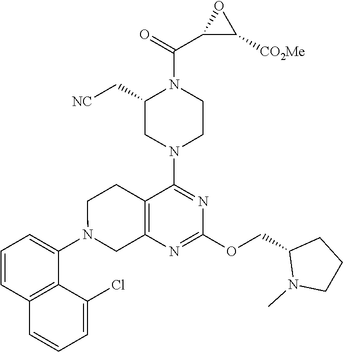 [SMILES: COC(=O)[C@H]1O[C@H]1C(=O)N1CCN(c2nc(OC[C@@H]3CCCN3C)nc3c2CCN(c2cccc4cccc(Cl)c24)C3)C[C@@H]1CC#N] |
| 288 | 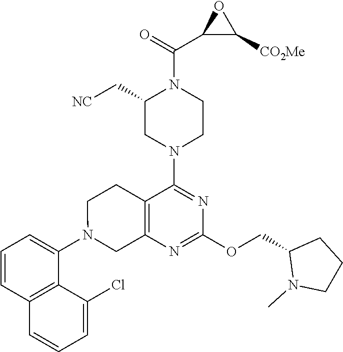 [SMILES: COC(=O)[C@@H]1O[C@@H]1C(=O)N1CCN(c2nc(OC[C@@H]3CCCN3C)nc3c2CCN(c2cccc4cccc(Cl)c24)C3)C[C@@H]1CC#N] |
| 289 | 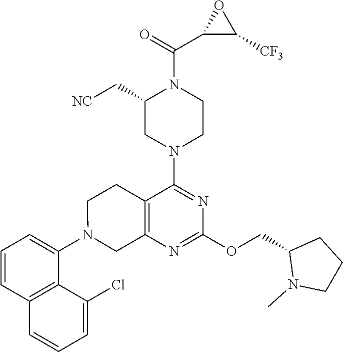 [SMILES: CN1CCC[C@H]1COc1nc2c(c(N3CCN(C(=O)[C@@H]4O[C@@H]4C(F)(F)F)[C@@H](CC#N)C3)n1)CCN(c1cccc3cccc(Cl)c13)C2] |
| 290 | 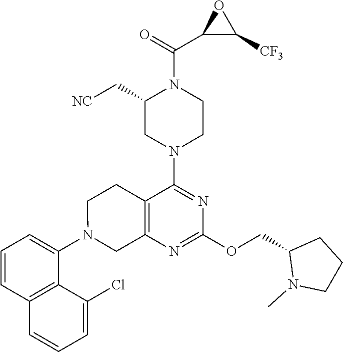 [SMILES: CN1CCC[C@H]1COc1nc2c(c(N3CCN(C(=O)[C@H]4O[C@H]4C(F)(F)F)[C@@H](CC#N)C3)n1)CCN(c1cccc3cccc(Cl)c13)C2] |
| 291 | 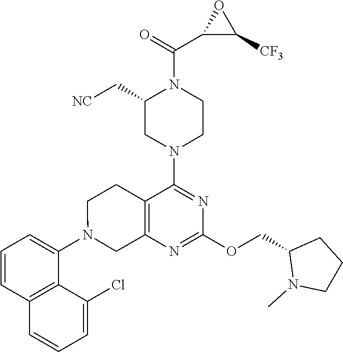 [SMILES: CN1CCC[C@H]1COc1nc2c(c(N3CCN(C(=O)[C@@H]4O[C@H]4C(F)(F)F)[C@@H](CC#N)C3)n1)CCN(c1cccc3cccc(Cl)c13)C2] |
| 292 | 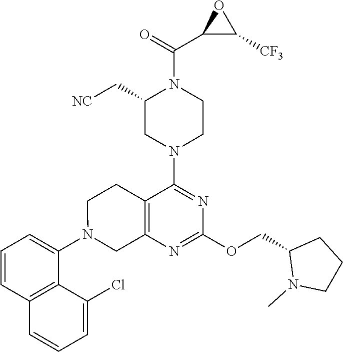 [SMILES: CN1CCC[C@H]1COc1nc2c(c(N3CCN(C(=O)[C@H]4O[C@@H]4C(F)(F)F)[C@@H](CC#N)C3)n1)CCN(c1cccc3cccc(Cl)c13)C2] |
| 293 | 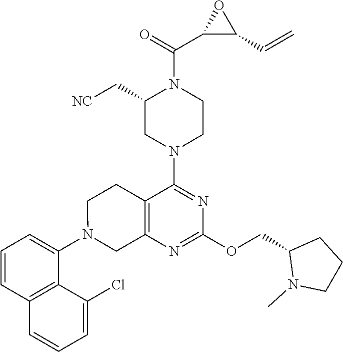 [SMILES: C=C[C@H]1O[C@H]1C(=O)N1CCN(c2nc(OC[C@@H]3CCCN3C)nc3c2CCN(c2cccc4cccc(Cl)c24)C3)C[C@@H]1CC#N] |
| 294 | 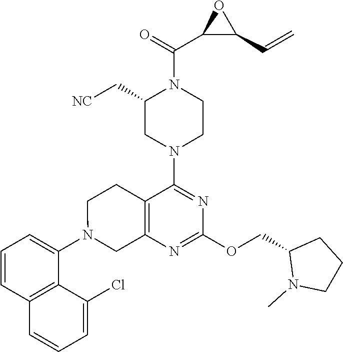 [SMILES: C=C[C@@H]1O[C@@H]1C(=O)N1CCN(c2nc(OC[C@@H]3CCCN3C)nc3c2CCN(c2cccc4cccc(Cl)c24)C3)C[C@@H]1CC#N] |
| 295 | 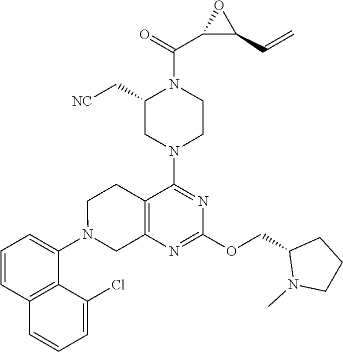 [SMILES: C=C[C@@H]1O[C@H]1C(=O)N1CCN(c2nc(OC[C@@H]3CCCN3C)nc3c2CCN(c2cccc4cccc(Cl)c24)C3)C[C@@H]1CC#N] |
| 296 | 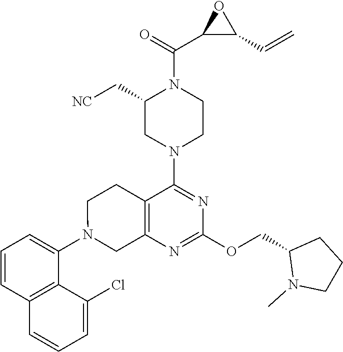 [SMILES: C=C[C@H]1O[C@@H]1C(=O)N1CCN(c2nc(OC[C@@H]3CCCN3C)nc3c2CCN(c2cccc4cccc(Cl)c24)C3)C[C@@H]1CC#N] |
| 297 | 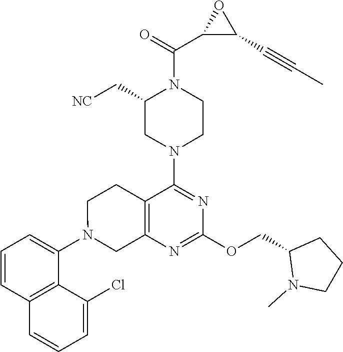 [SMILES: CC#C[C@H]1O[C@H]1C(=O)N1CCN(c2nc(OC[C@@H]3CCCN3C)nc3c2CCN(c2cccc4cccc(Cl)c24)C3)C[C@@H]1CC#N] |
| 298 | 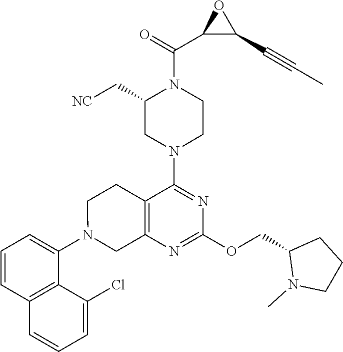 [SMILES: CC#C[C@@H]1O[C@@H]1C(=O)N1CCN(c2nc(OC[C@@H]3CCCN3C)nc3c2CCN(c2cccc4cccc(Cl)c24)C3)C[C@@H]1CC#N] |
| 299 | 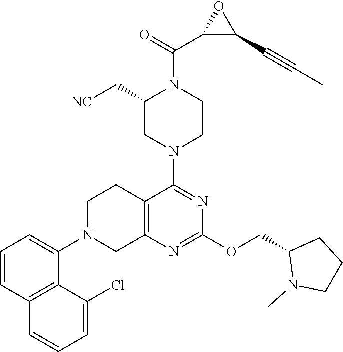 [SMILES: CC#C[C@@H]1O[C@H]1C(=O)N1CCN(c2nc(OC[C@@H]3CCCN3C)nc3c2CCN(c2cccc4cccc(Cl)c24)C3)C[C@@H]1CC#N] |
| 300 | 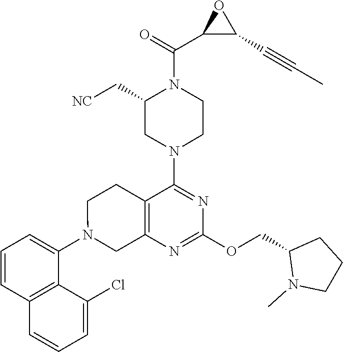 [SMILES: CC#C[C@H]1O[C@@H]1C(=O)N1CCN(c2nc(OC[C@@H]3CCCN3C)nc3c2CCN(c2cccc4cccc(Cl)c24)C3)C[C@@H]1CC#N] |

In any embodiment herein, such an embodiment does not include a compound as disclosed in WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 or WO 2013155223, or McGregor et al., Biochem., 56(25): 3178-3183 (2017).

Further provided is a Ras protein comprising a covalent bond to a compound of the present invention. In some embodiments, a conjugate, or a salt thereof, is provided wherein a Ras protein is covalently bound to a Ras binding moiety through a linker and a selective cross-linker, as those terms are defined herein, wherein said covalent bond is between the selective cross-linker and the Ras protein.

In some embodiments, a conjugate, or salt thereof, has the structure of Formula XIX:

A-LB-C Formula XIX

wherein A is a Ras binding moiety, such as a compound of Formula II, Formula III, Formula IV, or Formula V;

LB is a linker, such as a linker of Formula VI, VIIe, VIIf, or VIII, bound to a selective cross-linking group; and

C is a Ras protein, wherein C is covalently bound to B.

In some embodiments regarding a conjugate or salt thereof, the selective cross-linking group is bound to the Ras protein through a covalent bond to a carboxyl group of a Ras protein, such as a human mutant K-Ras protein, human mutant H-Ras protein, or human mutant N-Ras protein. In some embodiments, the Ras protein is K-Ras G12D, K-Ras G13D, or K-Ras G12S. In some embodiments, the carboxyl group of a residue of the Ras protein is the carboxyl group of an aspartic acid residue at the mutated position corresponding to position 12 or 13 of human wild-type K-Ras (SEQ ID NO: 1).

In some embodiments, a conjugate, or salt thereof, comprises a Ras protein covalently bound to a selective cross-linking group, which selective cross-linking group is bound to a Ras binding moiety through a linker, wherein the selective cross-linking group is a carbodiimide, an aminooxazoline, a chloroethyl urea, an aziridine, a trifluoromethyl ketone, a boronic acid, a boronic ester, an N-ethoxycarbonyl-2-ethoxy-1,2-dihydroquinoline (EEDQ), an iso-EEDQ, an epoxide, an oxazolium, or a glycal. In some embodiments, a selective cross-linking group is a carbodiimide, an aminooxazoline, a chloroethyl urea, an aziridine, a trifluoromethyl ketone, a boronic acid, a boronic ester, an epoxide, or a glycal. In some embodiments, a selective cross-linking group is a carbodiimide, an aminooxazoline, a chloroethyl urea, or an aziridine.

In some embodiments, a conjugate, or a salt thereof, comprises a linker selected from the group consisting of:

(a)

-A¹-(B¹)ₐ-(C¹)_{b}-(B²)_{c}-(D)-(B³)_{d}-(C²)ₑ-(B⁴)_{f}-A²- Formula VI

where A¹ is a bond between the linker and the Ras binding moiety; A² is a bond between the selective cross-linking group and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; a, b, c, d, e, and f are each, independently, 0 or 1; and D is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₂₋₆ heteroarylene, optionally substituted C₃₋₈ cycloalkylene, optionally substituted C₆₋₁₂ arylene, optionally substituted C₂-C₁₀ polyethylene glycol, or optionally substituted C₁₋₁₀ heteroalkylene, or a chemical bond linking A¹-(B¹)ₐ-(C¹)_{b}-(B²)_{c}- to -(B³)_{d}-(C²)ₑ-(B⁴)_{f}-A²;

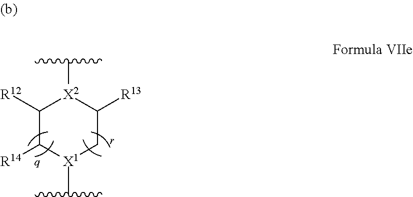

wherein q and r are, independently, 0, 1, or 2;

X¹ and X² are, independently, N or CH;

R¹² and R¹³ are, independently, hydrogen, optionally substituted C₁-C₆ alkyl or optionally substituted C₁-C₆ heteroalkyl; and

R¹⁴ is hydrogen, optionally substituted C₁-C₆ alkyl, or optionally substituted C₁-C₆ heteroalkyl, wherein R¹⁴ optionally comprises a bond to A;

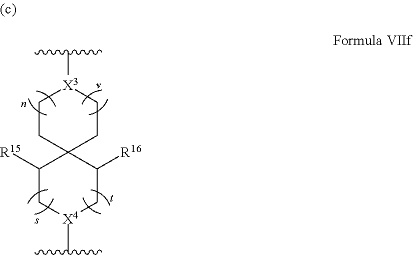

wherein s, t, u, and v are, independently, 0, 1, or 2;

X³ and X4 are, independently, N or CH; and

R¹⁵ and R^{1G} are, independently, hydrogen, optionally substituted C₁-C₆ alkyl or optionally substituted C₁-C₆ heteroalkyl; and

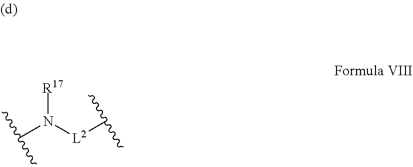

wherein R¹⁷ is hydrogen or optionally substituted C₁-C₆ alkyl; and

L² is optionally substituted C₁-C₄ alkylene or optionally substituted C₃-C₆ cycloalkylene.

Further provided is a method of producing a conjugate comprising contacting a Ras protein with a compound of the present invention, or a pharmaceutically acceptable salt thereof, or a pharmaceutical composition of such a compound or salt, under conditions sufficient for the compound to react covalently with the Ras protein. Also provided is method of producing a conjugate, the method comprising contacting a Ras protein with a compound of the present invention, or a pharmaceutically acceptable salt thereof, or a pharmaceutical composition of such a compound or salt, under conditions suitable to permit conjugate formation. Conjugates produced by such methods are also provided.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the disclosure can be synthesized using the methods described below as well as in the Examples below, together with synthetic methods known in the art of synthetic organic chemistry, or variations thereon as appreciated by those skilled in the art. These methods include but are not limited to those methods described below as well as in the Examples section.

The schemes below illustrate synthetic routes to append a selective cross-linking group (B) onto an intermediate comprised of a Ras binding moiety and linker (A-L). While only one A-L is shown, any appropriate Ras binding moiety and linker may be selected, such as from structures described in WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223, the Ras binding moieties of which are herein incorporated by reference. In view of the disclosures herein as well as general knowledge, persons of skill in the art will understand how a cross-linking group of a compound of these references may be replaced with a selective cross-linking group of the present invention.

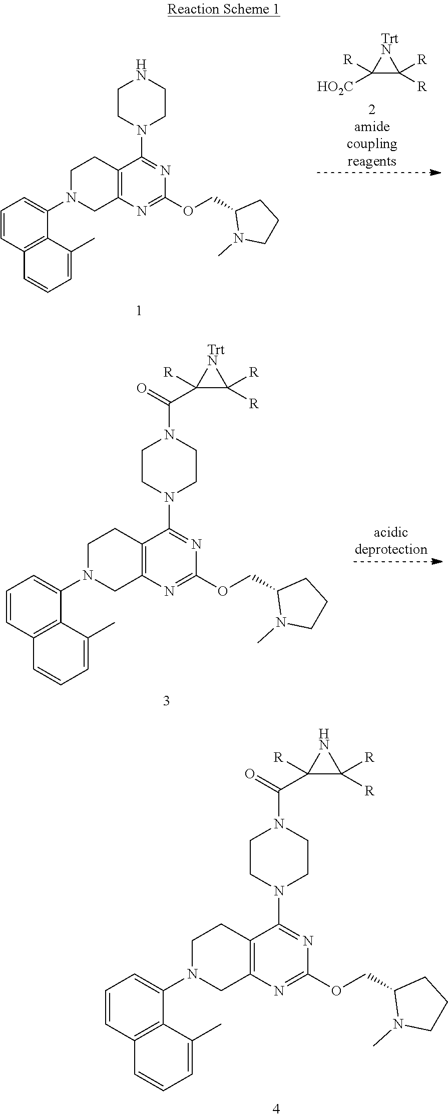

As shown in Scheme 1, compounds of type 4 may be prepared by the reaction of an appropriate amine such as compound 1 with a carboxylic acid such as compound 2 in the presence of standard amide coupling reagents, followed by trityl deprotection under acidic conditions.

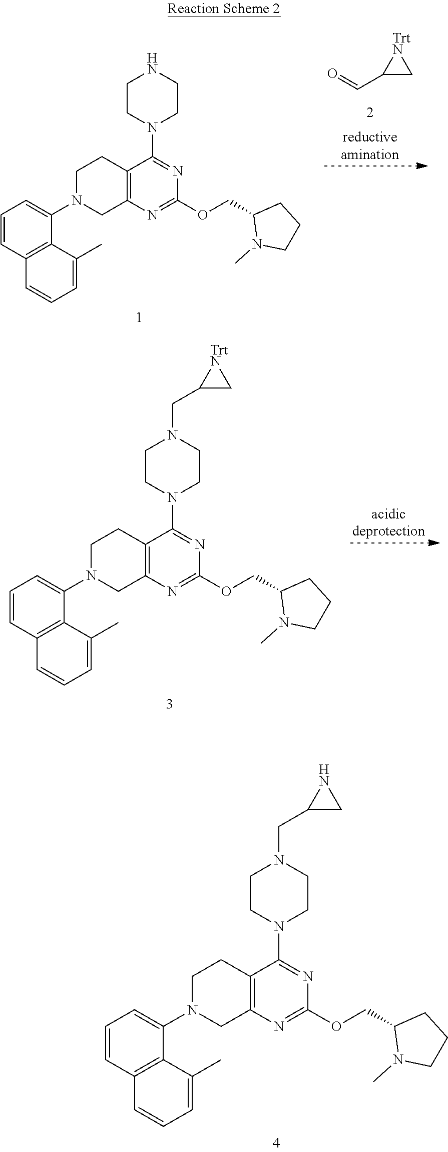

As shown in Scheme 2, compounds of type 4 may be prepared by the reductive amination of an appropriate amine such as compound 1 with an aldehyde such as compound 2, followed by trityl deprotection under acidic conditions.

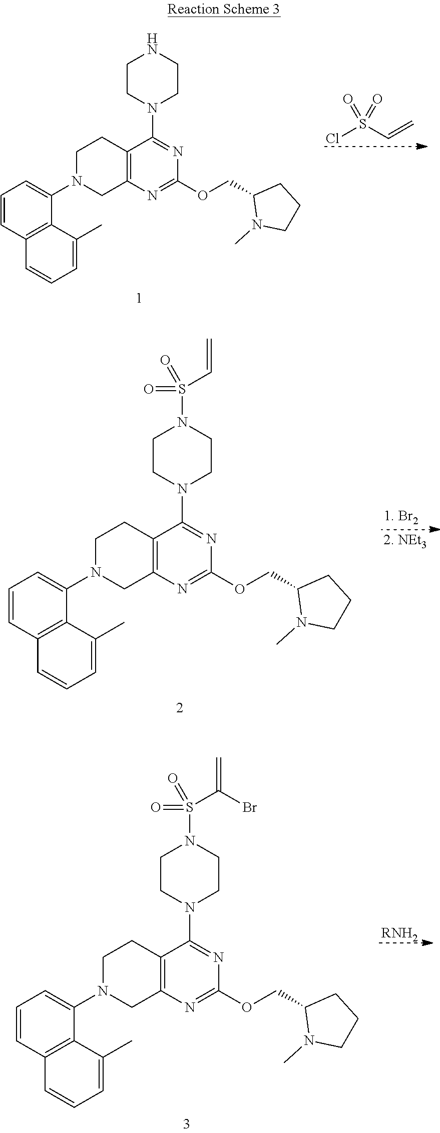

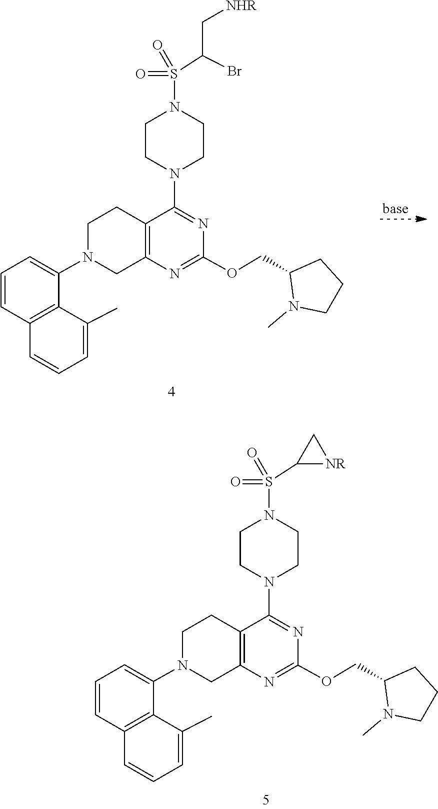

As shown in Scheme 3, compounds of type 3 may be prepared by the reaction of an appropriate amine such as compound 1 with vinylsulfonyl chloride followed by dibromination of the alkene and elimination using a suitable amine base. Reaction of compounds of type 3 with an appropriate primary amine produces compounds of type 4, which may be converted to compounds of type 5 in the presence of base.

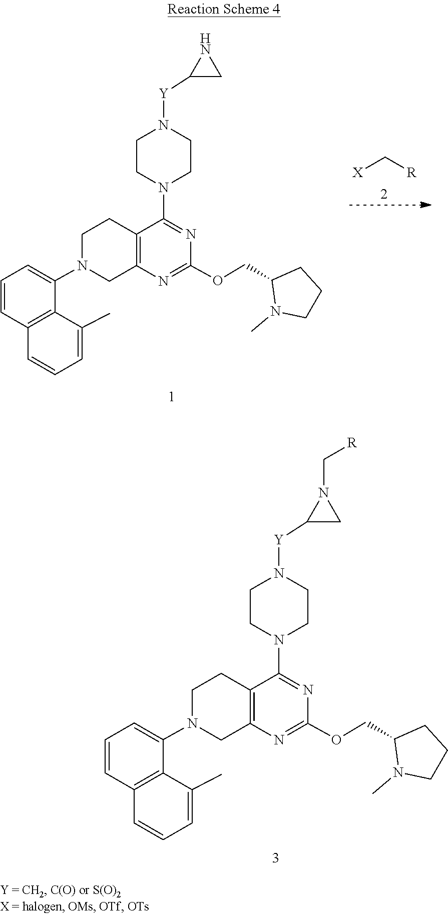

As shown in Scheme 4, compounds of type 3 may be prepared by the reaction of an appropriate amine such as compound 1 with a suitable alkyl halide or other leaving group, such as compounds of type 2.

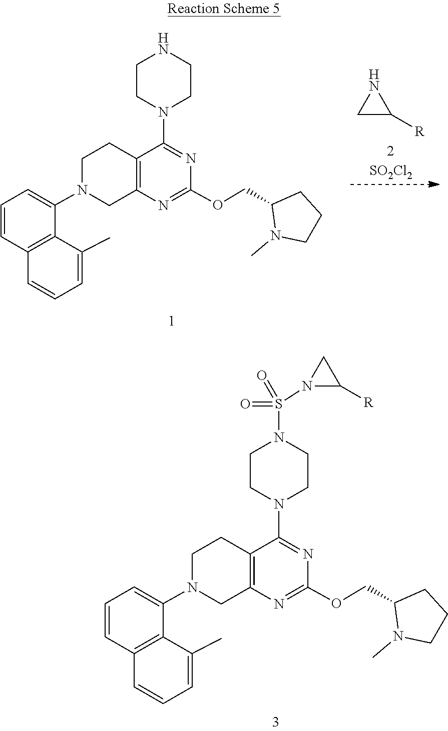

As shown in Scheme 5, compounds of type 3 may be prepared by the reaction of an appropriate amine such as compound 1 with sulfuryl chloride and an amine such as compound 2.

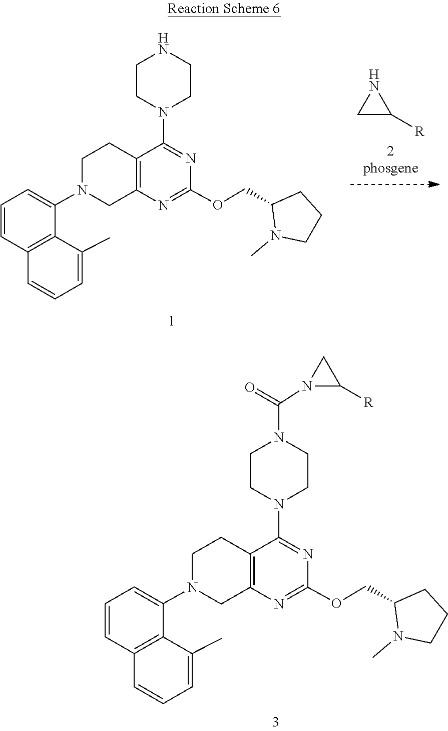

As shown in Scheme 6, compounds of type 3 may be prepared by the reaction of an appropriate amine such as compound 1 with phosgene and an amine such as compound 2.

### Pharmaceutical Compositions and Methods of Administration

As used herein, the term “pharmaceutical composition” refers to an active compound, formulated together with one or more pharmaceutically acceptable excipients. In some embodiments, a compound is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Excipients include, but are not limited to: butylated optionally substituted hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients.

Compounds described or depicted herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described here that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals, at any stage of development. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., provided compositions) that partially or completely alleviates, ameliorates, relives, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compounds, or a pharmaceutically acceptable salt thereof, described herein may be present in amounts totaling 1-95% by weight of the total weight of a composition, such as a pharmaceutical composition. The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

Compounds of the invention, or a pharmaceutically acceptable salt thereof, may be prepared and used as pharmaceutical compositions comprising a therapeutically effective amount of a compound, or a pharmaceutically acceptable salt thereof, described herein and a pharmaceutically acceptable carrier or excipient, as is well known in the art. In some embodiments, a composition includes at least two different pharmaceutically acceptable excipients or carriers.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677, which is herein incorporated by reference.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, of a combination therapy, as described herein, may be formulated in a variety of ways that are known in the art. For example, the first and second agents of the combination therapy may be formulated together or separately.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds, or a pharmaceutically acceptable salt thereof, of the combination of the invention will depend on the nature of the compound, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Methods of Use

In some embodiments, the invention discloses a method of treating a disease or disorder that is characterized by aberrant Ras activity due to a Ras mutant. In some embodiments, the disease or disorder is a cancer. In some embodiments, the cancer is colorectal cancer, non-small cell lung cancer, or small cell lung cancer. In some embodiments, the aberrant Ras activity is due to Ras G12D mutation. In some embodiments, the aberrant Ras activity is due to a K-Ras G12D mutation. In some embodiments, the aberrant Ras activity is due to Ras G13D mutation. In some embodiments, the aberrant Ras activity is due to a K-Ras G13D mutation. In some embodiments, the aberrant Ras activity is due to a Ras G12S mutation. In some embodiments, the aberrant Ras activity is due to a K-Ras G12S mutation. Other Ras mutations are described herein.

Also provided is a method of treating cancer in a subject in need thereof, the method comprising administering to the subject a therapeutically effective amount of a compound of the present invention, or a pharmaceutically acceptable salt thereof, or a pharmaceutical composition comprising such a compound or salt. In some embodiments, the cancer is colorectal cancer, non-small cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. Also provided is a method of treating a Ras protein-related disorder in a subject in need thereof, the method comprising administering to the subject a therapeutically effective amount of a compound of the present invention, or a pharmaceutically acceptable salt thereof, or a pharmaceutical composition comprising such a compound or salt. In some embodiments, the cancer comprises a Ras mutation, such as a Ras mutation described herein. In some embodiments, the Ras mutation is K-Ras G12D, K-Ras G13D, or K-Ras G12S.

In some embodiments, the compounds of the present invention or pharmaceutically acceptable salts thereof, pharmaceutical compositions comprising such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions comprising such compounds or salts, and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example:
Cardiac, for example: sarcoma (angiosarcoma, fibrosarcoma, rhabdomyosarcoma, iposarcoma), myxoma, rhabdomyoma, fibroma, lipoma and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors, neurofibromatosis type 1, spinal cord neurofibroma, meningioma, glioma, sarcoma;
Gynecological, for example: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

Also provided is a method of inhibiting a Ras protein in a cell, the method comprising contacting the cell with an effective amount of a compound of the present invention, or a pharmaceutically acceptable salt thereof. A method of inhibiting RAF-Ras binding, the method comprising contacting the cell with an effective amount of a compound of the present invention, or a pharmaceutically acceptable salt thereof. The cell may be a cancer cell. The cancer cell may be, for example, a colorectal cancer cell, a non-small cell lung cancer cell, a pancreatic cancer cell, a appendiceal cancer cell, a melanoma cell, an acute myeloid leukemia cell, a small bowel cancer cell, an ampullary cancer cell, a germ cell cancer cell, a cervical cancer cell, a cancer cell of unknown primary origin, an endometrial cancer cell, an esophagogastric cancer cell, a GI neuroendocrine cancer cell, an ovarian cancer cell, a sex cord stromal tumor cancer cell, a hepatobiliary cancer cell, or a bladder cancer cell. In some embodiments, the cancer is appendiceal, endometrial or melanoma.

### Combination Therapy

The present disclosure also provides methods for combination therapies in which an agent known to modulate other pathways, or other components of the same pathway, or even overlapping sets of targets, are used in combination with a compound of the present disclosure, or a pharmaceutically acceptable salt thereof. In one aspect, such therapy includes but is not limited to the combination of one or more compounds of the disclosure with antiproliferative agents, chemotherapeutic agents, therapeutic antibodies, and radiation treatment, to provide a synergistic or additive therapeutic effect. An example of other pharmaceuticals to combine with the compounds, or a pharmaceutically acceptable salt thereof, described herein would include pharmaceuticals for the treatment of the same indication. Another example of a potential pharmaceutical to combine with compounds, or a pharmaceutically acceptable salt thereof, described herein would include pharmaceuticals for the treatment of different yet associated or related symptoms or indications.

As used herein, the term “combination therapy” refers to those situations in which a subject is simultaneously exposed to two or more therapeutic regimens (e.g., two or more compounds, such as compounds of this invention). In some embodiments, two or more compounds may be administered simultaneously; in some embodiments, such compounds may be administered sequentially; in some embodiments, such compounds are administered in overlapping dosing regimens. In some embodiments, a combination therapeutic regimen employs two therapeutic agents, one compound of the present invention and a second selected from the therapeutic agents described herein. In some embodiments, a combination therapeutic regimen employs three therapeutic agents, one compound of the present invention and two selected from the therapeutic agents described herein. In some embodiments, a combination therapeutic regiment employs four or more therapeutic agents, one compound of the present invention and three selected from the therapeutic agents described herein. For example, a combination therapy may entail a Ras inhibitor as described herein, a MEK inhibitor, and a SHP2 inhibitor; a Ras inhibitor as described herein, a MEK inhibitor, and a SOS1 inhibitor; or a RAS inhibitor, a PDL-1 inhibitor, and a SHP2 inhibitor.

In this Combination Therapy section, all references are incorporated by reference for the agents described, whether explicitly stated as such or not.

In some embodiments, a compound of the present invention is used in combination with an EGFR inhibitor. In some embodiments, a compound of the present invention may be used in combination with an inhibitor of a member downstream of a Receptor Tyrosine Kinase (RTK)/Growth Factor Receptor, such a SHP2 inhibitor, a SOS1 inhibitor, a Raf inhibitor, a MEK inhibitor, an ERK inhibitor, a PI3K inhibitor, a PTEN inhibitor, an AKT inhibitor, or an mTORC1 inhibitor. Examples of these inhibitors are provided below.

In some embodiments, a compound of the present invention may be used in combination with a second Ras inhibitor. In some embodiments, the Ras inhibitor targets Ras in its active, or GTP-bound state. In some embodiments, the Ras inhibitor targets Ras in its inactive, or GDP-bound state, such as AMG 510, MRTX1257, MRTX849, JNJ-74699157, LY3499446, or ARS-1620.

Many chemotherapeutics are presently known in the art and can be used in combination with the compounds of the disclosure. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (Imatinib Mesylate), Kyprolis® (carfilzomib), Velcade® (bortezomib), Casodex® (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXANTM™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex®, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel and docetaxel; retinoic acid; esperamicins; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxy tamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; Xeloda®; ibandronate; camptothecin-11 (CPT-11); topoisomerase inhibitor RFS 2000; difluoromethylornithine (DMFO).

Where desired, the compounds or pharmaceutical composition of the present disclosure can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV

(chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

This disclosure further relates to a method for using the compounds or pharmaceutical compositions provided herein, in combination with radiation therapy for inhibiting abnormal cell growth or treating the hyperproliferative disorder in the mammal. Techniques for administering radiation therapy are known in the art, and these techniques can be used in the combination therapy described herein. The administration of the compound of the disclosure in this combination therapy can be determined as described herein.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, 1-131, 1-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as 1-125, 1-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

The compounds or pharmaceutical compositions of the disclosure can be used in combination with an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors, MMP-9 (matrix-metalloproteinase 9) inhibitors, and COX-11 (cyclooxygenase 11) inhibitors, can be used in conjunction with a compound of the disclosure and pharmaceutical compositions described herein. Anti-angiogenesis agents include, for example, rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO 96/33172, WO 96/27583, EP0818442, EP1004578, WO 98/07697, WO 98/03516, WO 98/34918, WO 98/34915, WO 98/33768, WO 98/30566, EP606046, WO 90/05719, WO 99/52910, WO 99/52889, WO 99/29667, WO1999007675, EP1786785, EP1181017, US20090012085, U.S. Pat. Nos. 5,863,949, 5,861,510, and EP0780386. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors useful in the disclosure are AG-3340, RO 32-3555, and RS 13-0830.

The present compounds may also be used in co-therapies with other anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-Ia, interferon beta-Ib, interferon gamma, natural interferon gamma-Ia, interferon gamma-Ib, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburi embodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aetema), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

In some embodiments, the anti-cancer agent is a HER2 inhibitor. Non-limiting examples of HER2 inhibitors include monoclonal antibodies such as trastuzumab (Herceptin®) and pertuzumab (Perjeta); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

The compounds of the invention may further be used with VEGFR inhibitors. Other compounds described in the following patents and patent applications can be used in combination therapy: U.S. Pat. No. 6,258,812, US 2003/0105091, WO 01/37820, U.S. Pat. No. 6,235,764, WO 01/32651, U.S. Pat. Nos. 6,630,500, 6,515,004, 6,713,485, 5,521,184, 5,770,599, 5,747,498, WO 02/68406, WO 02/66470, WO 02/55501, WO 04/05279, WO 04/07481, WO 04/07458, WO 04/09784, WO 02/59110, WO 99/45009, WO 00/59509, WO 99/61422, U.S. Pat. No. 5,990,141, WO 00/12089, and WO 00/02871.

In some embodiments, the combination comprises a composition of the present invention in combination with at least one anti-angiogenic agent. Agents are inclusive of, but not limited to, in vitro synthetically prepared chemical compositions, antibodies, antigen binding regions, radionuclides, and combinations and conjugates thereof. An agent can be an agonist, antagonist, allosteric modulator, toxin or, more generally, may act to inhibit or stimulate its target (e.g., receptor or enzyme activation or inhibition), and thereby promote cell death or arrest cell growth.

Exemplary anti-angiogenic agents include ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix (panitumumab), IRESSA™ (gefitinib), TARCEVA™ (erlotinib), anti-AngI and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). The pharmaceutical compositions of the present invention can also include one or more agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor “c-met”.

Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto).

Additional anti-angiogenic/anti-tumor agents include: SD-7784 (Pfizer, USA); cilengitide. (Merck KGaA, Germany, EPO 770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol, (EntreMed, USA); TLC ELL-12, (Elan, Ireland); anecortave acetate, (Alcon, USA); alpha-D148 Mab, (Amgen, USA); CEP-7055, (Cephalon, USA); anti-Vn Mab, (Crucell, Netherlands) DACantiangiogenic, (ConjuChem, Canada); Angiocidin, (InKine Pharmaceutical, USA); KM-2550, (Kyowa Hakko, Japan); SU-0879, (Pfizer, USA); CGP-79787, (Novartis, Switzerland, EP 970070); ARGENT technology, (Ariad, USA); YIGSR-Stealth, (Johnson & Johnson, USA); fibrinogen-E fragment, (BioActa, UK); angiogenesis inhibitor, (Trigen, UK); TBC-1635, (Encysive Pharmaceuticals, USA); SC-236, (Pfizer, USA); ABT-567, (Abbott, USA); Metastatin, (EntreMed, USA); angiogenesis inhibitor, (Tripep, Sweden); maspin, (Sosei, Japan); 2-methoxyestradiol, (Oncology Sciences Corporation, USA); ER-68203-00, (IV AX, USA); Benefin, (Lane Labs, USA); Tz-93, (Tsumura, Japan); TAN-1120, (Takeda, Japan); FR-111142, (Fujisawa, Japan, JP 02233610); platelet factor 4, (RepliGen, USA, EP 407122); vascular endothelial growth factor antagonist, (Borean, Denmark); bevacizumab (pINN), (Genentech, USA); angiogenesis inhibitors, (SUGEN, USA); XL 784, (Exelixis, USA); XL 647, (Exelixis, USA); MAb, alpha5beta3 integrin, second generation, (Applied Molecular Evolution, USA and MedImmune, USA); gene therapy, retinopathy, (Oxford BioMedica, UK); enzastaurin hydrochloride (USAN), (Lilly, USA); CEP 7055, (Cephalon, USA and Sanofi-Synthelabo, France); BC 1, (Genoa Institute of Cancer Research, Italy); angiogenesis inhibitor, (Alchemia, Australia); VEGF antagonist, (Regeneron, USA); rBPI 21 and BPI-derived antiangiogenic, (XOMA, USA); PI 88, (Progen, Australia); cilengitide (pINN), (Merck KGaA, German; Munich Technical University, Germany, Scripps Clinic and Research Foundation, USA); cetuximab (INN), (Aventis, France); AVE 8062, (Ajinomoto, Japan); AS 1404, (Cancer Research Laboratory, New Zealand); SG 292, (Telios, USA); Endostatin, (Boston Childrens Hospital, USA); ATN 161, (Attenuon, USA); ANGIOSTATIN, (Boston Childrens Hospital, USA); 2-methoxyestradiol, (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXIGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenesis, (EntreMed, USA); urokinase plasminogen activator inhibitor, (Dendreon, USA); oglufanide (pINN), (Melmotte, USA); HIF-Ialfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol, (EntreMed, USA); anginex, (Maastricht University, Netherlands, and Minnesota University, USA); ABT 510, (Abbott, USA); AAL 993, (Novartis, Switzerland); VEGI, (ProteomTech, USA); tumor necrosis factor-alpha inhibitors, (National Institute on Aging, USA); SU 11248, (Pfizer, USA and SUGEN USA); ABT 518, (Abbott, USA); YH16, (Yantai Rongchang, China); S-3APG, (Boston Childrens Hospital, USA and EntreMed, USA); MAb, KDR, (ImClone Systems, USA); MAb, alpha5 betaI, (Protein Design, USA); KDR kinase inhibitor, (Celltech Group, UK, and Johnson & Johnson, USA); GFB 116, (South Florida University, USA and Yale University, USA); CS 706, (Sankyo, Japan); combretastatin A4 prodrug, (Arizona State University, USA); chondroitinase AC, (IBEX, Canada); BAY RES 2690, (Bayer, Germany); AGM 1470, (Harvard University, USA, Takeda, Japan, and TAP, USA); AG 13925, (Agouron, USA); Tetrathiomolybdate, (University of Michigan, USA); GCS 100, (Wayne State University, USA) CV 247, (Ivy Medical, UK); CKD 732, (Chong Kun Dang, South Korea); MAb, vascular endothelium growth factor, (Xenova, UK); irsogladine (INN), (Nippon Shinyaku, Japan); RG 13577, (Aventis, France); WX 360, (Wilex, Germany); squalamine (pINN), (Genaera, USA); RPI 4610, (Sirna, USA); cancer therapy, (Marinova, Australia); heparanase inhibitors, (InSight, Israel); KL 3106, (Kolon, South Korea); Honokiol, (Emory University, USA); ZK CDK, (Schering AG, Germany); ZK Angio, (Schering AG, Germany); ZK 229561, (Novartis, Switzerland, and Schering AG, Germany); XMP 300, (XOMA, USA); VGA 1102, (Taisho, Japan); VEGF receptor modulators, (Pharmacopeia, USA); VE-cadherin-2 antagonists, (ImClone Systems, USA); Vasostatin, (National Institutes of Health, USA); vaccine, Flk-1, (ImClone Systems, USA); TZ 93, (Tsumura, Japan); TumStatin, (Beth Israel Hospital, USA); truncated soluble FLT 1 (vascular endothelial growth factor receptor 1), (Merck & Co, USA); Tie-2 ligands, (Regeneron, USA); and, thrombospondin 1 inhibitor, (Allegheny Health, Education and Research Foundation, USA).

Autophagy inhibitors include, but are not limited to chloroquine, 3-methyladenine, hydroxychloroquine (Plaquenil™), bafilomycin AI, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

Additional pharmaceutically active compounds/agents that can be used in the treatment of cancers and that can be used in combination with one or more compound of the present invention include: epoetin alfa; darbepoetin alfa; panitumumab; pegfilgrastim; palifermin; filgrastim; denosumab; ancestim; AMG 102; AMG 386; AMG 479; AMG 655; AMG 745; AMG 951; and AMG 706, or a pharmaceutically acceptable salt thereof.

In certain embodiments, a composition provided herein is conjointly administered with a chemotherapeutic agent. Suitable chemotherapeutic agents may include, natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs,

melphalan, and chlorambucil), ethylenimines and methylmelamines (e.g., hexaamethylmelaamine and thiotepa), CDK inhibitors (e.g., ribociclib, abemaciclib, palbociclib, seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., temsirolimus, everolimus, ridaforolimus, and sirolimus; see also below), KSP(Eg5) inhibitors (e.g., Array 520), DNA binding agents (e.g., Zalypsis), PI3K delta inhibitor (e.g., GS-1101 and TGR-1202), PI3K delta and gamma inhibitor (e.g., CAL-130), multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), P13K/Akt inhibitors (e.g., perifosine), Akt inhibitor (e.g., GSK-2141795), PKC inhibitors (e.g., enzastaurin), FTIs (e.g., Zamestra™), anti-CD138 (e.g., BT062), Torcl/2 specific kinase inhibitor (e.g., INK128), kinase inhibitor (e.g., GS-1101), ER/UPR targeting agent (e.g., MKC-3946), cFMS inhibitor (e.g., ARRY-382), JAK1/2 inhibitor (e.g., CYT387), PARP inhibitor (e.g., olaparib and veliparib (ABT-888)), and BCL-2 antagonist. Other chemotherapeutic agents may include mechlorethamine, camptothecin, ifosfamide, tamoxifen, raloxifene, gemcitabine, navelbine, sorafenib, or any analog or derivative variant of the foregoing.

Other mTOR inhibitors that may be combined with compounds of the present invention include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyl)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

The compounds of the present invention may also be used in combination with radiation therapy, hormone therapy, surgery and immunotherapy, which therapies are well known to those skilled in the art.

In certain embodiments, a pharmaceutical composition provided herein is conjointly administered with a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof. In a particular embodiment, the compounds of the present invention can also be used in combination with additional pharmaceutically active agents that treat nausea. Examples of agents that can be used to treat nausea include: dronabinol; granisetron; metoclopramide; ondansetron; and prochlorperazine; or a pharmaceutically acceptable salt thereof.

The compounds of the present invention may also be used in combination with an additional pharmaceutically active compound that disrupts or inhibits RAS-RAF-ERK or PI3K-AKT-TOR signaling pathways. In some combinations, the additional pharmaceutically active compound is a PD-1 or PD-L1 antagonist. The compounds or pharmaceutical compositions of the disclosure can also be used in combination with an amount of one or more substances selected from EGFR inhibitors, MEK inhibitors, PI3K inhibitors, AKT inhibitors, TOR inhibitors, McI-1 inhibitors, BCL-2 inhibitors, SHP2 inhibitors, proteasome inhibitors, and immune therapies, including monoclonal antibodies, immunomodulatory imides (IMiDs), anti-PD-1, anti-PDL-1, anti-CTLA4, anti-LAGI, and anti-OX40 agents, GITR agonists, CAR-T cells, and BiTEs.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Small molecule antagonists of EGFR include gefitinib, erlotinib (Tarceva®), osimertinib (Tagrisso®), and lapatinib (TykerB®). See e.g., Yan L, et. al, Pharmacogenetics and Pharmacogenomics In Oncology Therapeutic Antibody Development, BioTechniques 2005; 39(4): 565-8, and Paez J G, et. al, EGFR Mutations In Lung Cancer Correlation With Clinical Response To Gefitinib Therapy, Science 2004; 304(5676): 1497-500.

Non-limiting examples of small molecule EGFR inhibitors include any of the EGFR inhibitors described in the following patent publications, and all pharmaceutically acceptable salts and solvates of said EGFR inhibitors: European Patent Application EP 520722, published Dec. 30, 1992; European Patent Application EP 566226, published Oct. 20, 1993; PCT International Publication WO 96/33980, published Oct. 31, 1996; U.S. Pat. No. 5,747,498, issued May 5, 1998; PCT International Publication WO 96/30347, published Oct. 3, 1996; European Patent Application EP 787772, published Aug. 6, 1997; PCT International Publication WO 97/30034, published Aug. 21, 1997; PCT International Publication WO 97/30044, published Aug. 21, 1997; PCT International Publication WO 97/38994, published Oct. 23, 1997; PCT International Publication WO 97/49688, published Dec. 31, 1997; European Patent Application EP 837063, published Apr. 22, 1998; PCT International Publication WO 98/02434, published Jan. 22, 1998; PCT International Publication WO 97/38983, published Oct. 23, 1997; PCT International Publication WO 95/19774, published Jul. 27, 1995; PCT International Publication WO 95/19970, published Jul. 27, 1995; PCT International Publication WO 97/13771, published Apr. 17, 1997; PCT International Publication WO 98/02437, published Jan. 22, 1998; PCT International Publication WO 98/02438, published Jan. 22, 1998; PCT International Publication WO 97/32881, published Sep. 12, 1997; German Application DE 19629652, published Jan. 29, 1998; PCT International Publication WO 98/33798, published Aug. 6, 1998; PCT International Publication WO 97/32880, published Sep. 12, 1997; PCT International Publication WO 97/32880 published Sep. 12, 1997; European Patent Application EP 682027, published Nov. 15, 1995; PCT International Publication WO 97/02266, published January 23, 197; PCT International Publication WO 97/27199, published Jul. 31, 1997; PCT International Publication WO 98/07726, published Feb. 26, 1998; PCT International Publication WO 97/34895, published Sep. 25, 1997; PCT International Publication WO 96/31510, published Oct. 10, 1996; PCT International Publication WO 98/14449, published Apr. 9, 1998; PCT International Publication WO 98/14450, published Apr. 9, 1998; PCT International Publication WO 98/14451, published Apr. 9, 1998; PCT International Publication WO 95/09847, published Apr. 13, 1995; PCT International Publication WO 97/19065, published May 29, 1997; PCT International Publication WO 98/17662, published Apr. 30, 1998; U.S. Pat. No. 5,789,427, issued Aug. 4, 1998; U.S. Pat. No. 5,650,415, issued Jul. 22, 1997; U.S. Pat. No. 5,656,643, issued Aug. 12, 1997; PCT International Publication WO 99/35146, published Jul. 15, 1999; PCT International Publication WO 99/35132, published Jul. 15, 1999; PCT International Publication WO 99/07701, published Feb. 18, 1999; and PCT International Publication WO 92/20642 published Nov. 26, 1992. Additional non-limiting examples of small molecule EGFR inhibitors include any of the EGFR inhibitors described in Traxler, P., 1998, Exp. Opin. Ther. Patents 8(12): 1599-1625. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4).

Antibody-based EGFR inhibitors include any anti-EGFR antibody or antibody fragment that can partially or completely block EGFR activation by its natural ligand. Non-limiting examples of antibody-based EGFR inhibitors include those described in Modjtahedi, H., et al, 1993, Br. J. Cancer 67:247-253; Teramoto, T., et al, 1996, Cancer 77:639-645; Goldstein et al, 1995, Clin. Cancer Res. 1: 1311-1318; Huang, S. M., et al., 1999, Cancer Res. 15:59(8): 1935-40; and Yang, X., et al., 1999, Cancer Res. 59: 1236-1243. Thus, the EGFR inhibitor can be monoclonal antibody Mab E7.6.3 (Yang, 1999 supra), or Mab C225 (ATCC Accession No. HB-8508), or an antibody or antibody fragment having the binding specificity thereof.

MEK inhibitors include, but are not limited to, cobimetinib, trametinib, and binimetinib.

PI3K inhibitors include, but are not limited to, wortmannin, 17-hydroxywortmannin analogs described in WO 06/044453, 4-[2-(IH-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036,082 and WO 09/055,730), 2-Methyl-2-[4-[3-methyl-2-oxo-8-(quinolin-3-yl)-2,3-dihydroimidazo[4,5-c]quinolin-I-yl]phenyl]propionitrile (also known as BEZ 235 or NVP-BEZ 235, and described in PCT Publication No. WO 06/122806), (S)-I-(4-((2-(2-aminopyrinddin-5-yl)-7-methyl-4-morpholinothieno[3,2-d]pyriiTddin-6-yl)methyl)piperazin-I-yl)-2-hydroxypropan-1-one (described in PCT Publication No. WO 2008/070740), LY294002 (2-(4-Morpholinyl)-8-phenyl-4H-I-benzopyran-4-one available from Axon Medchem), PI 103 hydrochloride (3-[4-(4-morpholinylpyrido-[3′,2′:4,5]furo[3,2-d]pyrimidin-2-yl] phenol hydrochloride available from Axon Medchem), PIK 75 (N′-[(IE)-(6-bromoinddazo[I,2-a]pyridin-3-yl)methylene]-N,2-dimethyl-5-nitrobenzenesulfono-hydrazide hydrochloride available from Axon Medchem), PIK 90 (N-(7,8-dimethoxy-2,3-dihydro-imidazo[I,2-c]quinazolin-5-yl)-nicotinamide available from Axon Medchem), GDC-0941 bismesylate (2-(IH-Indazol-4-yl)-6-(4-methanesulfonyl-piperazin-1-ylmethyl)-4-mo holin-4-yl-thieno[3,2-d]pyrimidine bismesylate available from Axon Medchem), AS-252424 (5-[I-[5-(4-Fluoro-2-hydroxy-phenyl)-furan-2-yl]-meth-(Z)-ylidene]-thiazolidine-2,4-dione available from Axon Medchem), and TGX-221 (7-Methyl-2-(4-morpholinyl)-9-[I-(phenylamino)ethyl]-4H-pyrido-[1,2-a]pyrimidin-4-one available from Axon Medchem), XL-765, and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits AktI) (Barnett et al. (2005) Biochem. J., 385 (Pt. 2), 399-408); Akt-1-1,2 (inhibits AkI and 2) (Barnett et al. (2005) Biochem. J. 385 (Pt. 2), 399-408); API-59CJ-Ome (e.g., Jin et al. (2004) Br. J. Cancer 91, 1808-12); I-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134(12 Suppl), 3493S-3498S); perifosine (e.g., interferes with Akt membrane localization; Dasmahapatra et al. (2004) Clin. Cancer Res. 10(15), 5242-52, 2004); phosphatidylinositol ether lipid analogues (e.g., Gills and Dennis (2004) Expert. Opin. Investig. Drugs 13, 787-97); and triciribine (TCN or API-2 or NCI identifier: NSC 154020; Yang et al. (2004) Cancer Res. 64, 4394-9).

TOR inhibitors include, but are not limited to, inhibitors include AP-23573, CCI-779, everolimus, RAD-001, rapamycin, temsirolimus, ATP-competitive TORC1/TORC2 inhibitors, including PI-103, PP242, PP30 and Torin 1. Other TOR inhibitors in FKBP12 enhancer; rapamycins and derivatives thereof, including: CCI-779 (temsirolimus), RAD001 (Everolimus; WY 9409010) and AP23573; rapalogs, e.g., as disclosed in WO 98/02441 and WO 01/14387, e.g., AP23573, AP23464, or AP23841; 40-(2-hydroxyethyl)rapamycin, 40-[3-hydroxy(hydroxymethyl)methylpropanoate]-rapamycin (also called CC1779), 40-epi-(tetrazolyl)-rapamycin (also called ABT578), 32-deoxorapamycin, 16-pentynyloxy-32(S)-dihydrorapanycin, and other derivatives disclosed in WO 05005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; phosphorus-containing rapamycin derivatives (e.g., WO 05016252); 4H-I-benzopyran-4-one derivatives (e.g., WO 2005/056014).

Optional BRAF inhibitors that may be used in combination include, for example, vemurafenib, dabrafenib, and encorafenib.

MCI-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

Proteasome inhibitors include, but are not limited to, Kyprolis® (carfilzomib), Velcade® (bortezomib), and oprozomib.

Immune therapies include, but are not limited to, anti-PD-1 agents, anti-PDL-1 agents, anti-CTLA-4 agents, anti-LAGI agents, and anti-OX40 agents.

Monoclonal antibodies include, but are not limited to, Darzalex& (daratumumab), Herceptin® (trastuzumab), Avastin® (bevacizumab), Rituxan® (rituximab), Lucentis® (ranibizumab), and Eylea® (aflibercept).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al, Blood 110(1): 186-192 (2007), Thompson et al., Clin. Cancer Res. 13(6): 1757-1761 (2007), and Korman et al, International Application No. PCT/JP2006/309606 (publication no. WO 2006/121168 AI), each of which are expressly incorporated by reference herein, include: Yervoy™ (ipilimumab) or Tremelimumab (to CTLA-4), galiximab (to B7.1), BMS-936558 (to PD-1), MK-3475 (to PD-1) (pembrolizumab), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3), Ipilumumab (to CTLA-4). Immune therapies also include genetically engineered T-cells (e.g., CAR-T cells) and bispecific antibodies (e.g., BiTEs).

GITR agonists include, but are not limited to, GITR fusion proteins and anti-GITR antibodies (e.g., bivalent anti-GITR antibodies), such as, a GITR fusion protein described in U.S. Pat. No. 6,111,090, European Patent No. 090505B1, U.S. Pat. No. 8,586,023, PCT Publication Nos. WO 2010/003118 and 2011/090754, or an anti-GITR antibody described, e.g., in U.S. Pat. No. 7,025,962, European Patent No. 1947183B1, U.S. Pat. Nos. 7,812,135, 8,388,967, 8,591,886, European Patent No. EP 1866339, PCT Publication No. WO 2011/028683, PCT Publication No. WO 2013/039954, PCT Publication No. WO2005/007190, PCT Publication No. WO 2007/133822, PCT Publication No. WO2005/055808, PCT Publication No. WO 99/40196, PCT Publication No. WO 2001/03720, PCT Publication No. WO99/20758, PCT Publication No. WO2006/083289, PCT Publication No. WO 2005/115451, U.S. Pat. No. 7,618,632, and PCT Publication No. WO 2011/051726.

In some embodiments, the additional therapeutic agent is a SHP2 inhibitor. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N-SH2 and C-SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N-SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phophoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and PCT applications: WO2015107493; WO2015107494; WO201507495; WO2016203404; WO2016203405; WO2016203406; WO2011022440; WO2017156397; WO2017079723; WO2017211303; WO2012041524; WO2017211303; WO2019051084; WO2017211303; US20160030594; US20110281942; WO2010011666; WO2014113584; WO2014176488; WO2017100279; WO2019051469; U.S. Pat. No. 8,637,684; WO2007117699; WO2015003094; WO2005094314; WO2008124815; WO2009049098; WO2009135000; WO2016191328; WO2016196591; WO2017078499; WO2017210134; WO2018013597; WO2018129402; WO2018130928; WO20181309928; WO2018136264; WO2018136265; WO2018160731; WO2018172984; and WO2010121212, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TNO155. In some embodiments, the SHP2 inhibitor is RMC-4550. In some embodiments, the SHP2 inhibitor is RMC-4630. In some embodiments, the SHP2 inhibitor is JAB-3068. In some embodiments, the SHP2 inhibitor is RLY-1971.

In some embodiments, the additional therapeutic agent is selected from the group consisting of a HER2 inhibitor, a SHP2 inhibitor, a CDK4/6 inhibitor, an mTOR inhibitor, a SOS1 inhibitor, or a PD-L1 inhibitor. See, e.g., Hallin et al., Cancer Discovery, DOI: 10.1158/2159-8290 (Oct. 28, 2019) and Canon et al., Nature, 575:217(2019). In some embodiments, the additional therapeutic agent is selected from the group consisting of an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, and a CDK4/6 inhibitor, a HER2 inhibitor, or a combination thereof. In some embodiments, the additional therapeutic agents are a second Ras inhibitor and a PD-L1 inhibitor (i.e., triplet therapy).

The compounds described herein can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

As one aspect of the present invention contemplates the treatment of the disease/conditions with a combination of pharmaceutically active compounds that may be administered separately, the invention further relates to combining separate pharmaceutical compositions in kit form. The kit comprises two separate pharmaceutical compositions: a compound of the present invention, and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

In addition, it is to be understood that any embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any embodiment of the compositions of the invention can be excluded from any one or more claims, for any reason, whether related to the existence of prior art or not.

### Numbered Embodiments

[1] A compound having the structure of Formula I:

A-L-B Formula I

wherein A is a Ras binding moiety;

L is a linker; and

B is a selective cross-linking group,

or a pharmaceutically acceptable salt thereof,

wherein, upon contacting the compound, or a pharmaceutically acceptable salt thereof, with a sample containing a Ras protein, at least 20% of the Ras protein in the sample covalently reacts with the compound, or a pharmaceutically acceptable salt thereof, to form a conjugate.

[2] The compound, or a pharmaceutically acceptable salt thereof, of paragraph [1], wherein the Ras protein in the sample is a human H-Ras, a human N-Ras, a human K-Ras, or a combination thereof.

[3] The compound or a pharmaceutically acceptable salt thereof, of paragraphs [1] or [2], wherein the Ras protein in the sample is a mutant Ras protein.

[4] The compound, or a pharmaceutically acceptable salt thereof, of paragraph [1], wherein the Ras binding moiety is a human H-Ras binding moiety, a human N-Ras binding moiety, or a human K-Ras binding moiety.

[5] The compound, or a pharmaceutically acceptable salt thereof, of any one of paragraphs [2] to [4], wherein the Ras binding moiety is a K-Ras binding moiety and the Ras protein in the sample is a K-Ras protein.

[6] The compound, or a pharmaceutically acceptable salt thereof, of paragraph [5], wherein the K-Ras binding moiety interacts with a residue of a K-Ras Switch-II binding pocket of the K-Ras protein.

[7] The compound, or a pharmaceutically acceptable salt thereof, of paragraph [6], wherein the residue of a K-Ras Switch-II binding pocket is a residue of the K-Ras protein corresponding to V7, V8, V9, G10, A11, D12, K16, P34, T58, A59, G60, Q61, E62, E63, Y64, S65, R68, D69, Y71, M72, F78, I92, H95, Y96, Q99, I100, R102, or V103 of human wild-type K-Ras (SEQ ID NO: 1).

[8] The compound, or a pharmaceutically acceptable salt thereof, of any one of paragraphs [5] to [7], wherein the K-Ras binding moiety is the structure of any one of Formulas II-V.

[9] The compound, or a pharmaceutically acceptable salt thereof, of paragraph [8], wherein the K-Ras binding moiety is the structure of Formula II:

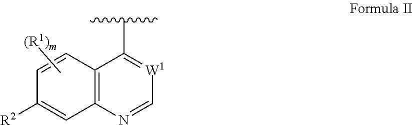

wherein m is 0, 1, 2, or 3;

W¹ is N or C, wherein C is optionally attached to the linker via an optionally substituted C₁-C₃ alkylene bridge or an optionally substituted C₁-C₃ heteroalkylene bridge;

each R¹ is, independently, CN, halo, hydroxy, optionally substituted C₁-C₆ alkyl, or optionally substituted C₁-C₆ heteroalkyl, or

R¹ is attached to the linker via a C₁-C₃ alkylene bridge or C₁-C₃ heteroalkylene bridge; and

R² is optionally substituted C₆-C₁₀ aryl or optionally substituted C₂-C₉ heteroaryl.

[10] The compound, or a pharmaceutically acceptable salt thereof, of paragraph [9], wherein the K-Ras binding moiety is the structure of Formula II-1:

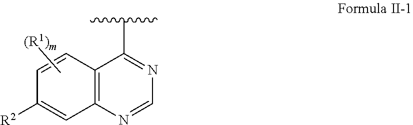

wherein m is 0, 1, 2, or 3;

each R¹ is, independently, CN, halo, hydroxy, optionally substituted C₁-C₆ alkyl, or optionally substituted C₁-C₆ heteroalkyl, or

R¹ is attached to the linker via a C₁-C₃ alkylene bridge or C₁-C₃ heteroalkylene bridge; and

R² is optionally substituted C₆-C₁₀ aryl or optionally substituted C₂-C₉ heteroaryl.

[11] The compound, or a pharmaceutically acceptable salt thereof, of paragraph [10], having the structure

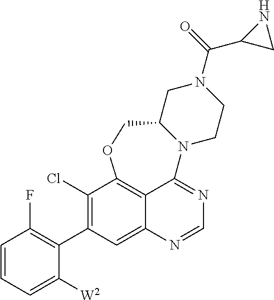

wherein W² is hydrogen or hydroxy.

[12] The compound, or a pharmaceutically acceptable salt thereof, of paragraph [9], wherein the K-Ras binding moiety is the structure of Formula II-2:

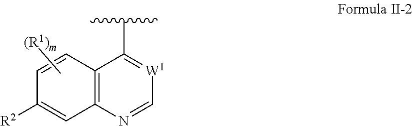

wherein m is 0, 1, 2, or 3;

W¹ is C attached to the linker via an optionally substituted C₁-C₃ alkylene bridge or an optionally substituted C₁-C₃ heteroalkylene bridge;

each R¹ is, independently, CN, halo, hydroxy, optionally substituted C₁-C₆ alkyl, or optionally substituted C₁-C₆ heteroalkyl, or

R¹ is attached to the linker via a C₁-C₃ alkylene bridge or C₁-C₃ heteroalkylene bridge; and

R² is optionally substituted C₆-C₁₀ aryl or optionally substituted C₂-C₉ heteroaryl.

[13] The compound, or a pharmaceutically acceptable salt thereof, of paragraph [8], wherein the K-Ras binding moiety is the structure of Formula III:

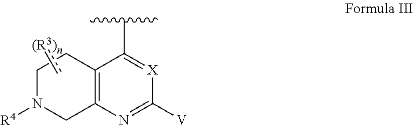

wherein n is 0, 1, 2, 3, 4, 5, or 6;

[symbol] represents a single bond or a double bond;

X is N or CR′, wherein R′ is hydrogen, or R′ is attached to the linker via an optionally substituted C₁-C₃ alkylene bridge, or optionally substituted C₁-C₃ heteroalkylene bridge;

V is CHR⁵, CR⁵R⁵, OR⁵, NHR⁵, or NR^{5a}R^{5b};

each R³ is, independently,

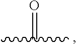 [SMILES: *=O]

optionally substituted C₁-C₆ alkyl, or optionally substituted C₁-C₆ heteroalkyl, or

R³ is attached to the linker via an optionally substituted C₁-C₃ alkylene bridge or optionally substituted C₁-C₃ heteroalkylene bridge;

R⁴ is optionally substituted C₆-C₁₀ aryl or optionally substituted C₂-C₉ heteroaryl;

each R⁵ is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl; and

each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl, or

R^{5a} and R^{5b}, together with the nitrogen atom to which each is attached, combine to form optionally substituted C₂-C₉ heterocyclyl;

provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge, and

further provided that when R³ is attached to the linker via an optionally substituted C₁-C₃ alkylene bridge or optionally substituted C₁-C₃ heteroalkylene bridge, R′ not is attached to the linker via an optionally substituted C₁-C₃ alkylene bridge or optionally substituted C₁-C₃ heteroalkylene bridge.

[14] The compound, or a pharmaceutically acceptable salt thereof, of paragraph [13], wherein the K-Ras binding moiety is the structure of Formula III-1:

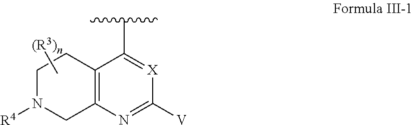

wherein n is 0, 1, 2, 3, 4, 5, or 6;

X is N or CR′, wherein R′ is hydrogen, or R′ is attached to the linker via an optionally substituted C₁-C₃ alkylene bridge or optionally substituted C₁-C₃ heteroalkylene bridge;

V is CHR⁵, CR⁵R⁵, OR⁵, NHR⁵, or NR^{5a}R^{5b};

each R³ is, independently, optionally substituted C₁-C₆ alkyl or optionally substituted C₁-C₆ heteroalkyl, or

R³ is attached to the linker via an optionally substituted C₁-C₃ alkylene bridge or optionally substituted C₁-C₃ heteroalkylene bridge;

R⁴ is optionally substituted C₆-C₁₀ aryl or optionally substituted C₂-C₉ heteroaryl;

each R⁵ is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl; and

each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl;

provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge, and

further provided that when R³ is attached to the linker via an optionally substituted C₁-C₃ alkylene bridge or optionally substituted C₁-C₃ heteroalkylene bridge, R′ not is attached to the linker via an optionally substituted C₁-C₃ alkylene bridge or optionally substituted C₁-C₃ heteroalkylene bridge.

[15] The compound, or a pharmaceutically acceptable salt thereof, of paragraph [14], wherein the K-Ras binding moiety is the structure of Formula III-1a:

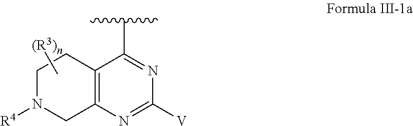

wherein n is 0, 1, 2, 3, 4, 5, or 6;

V is CHR⁵, CR⁵R⁵, OR⁵, NHR⁵, or NR^{5a}R^{5b};

each R³ is, independently, optionally substituted C₁-C₆ alkyl or optionally substituted C₁-C₆ heteroalkyl, or

R³ is attached to the linker via a C₁-C₃ alkylene bridge or C₁-C₃ heteroalkylene bridge;

R⁴ is optionally substituted C₆-C₁₀ aryl or C₂-C₉ heteroaryl;

each R⁵ is, independently, optionally substituted C₁-C₆ alkyl, optionally substituted C₁-C₆ heteroalkyl, optionally substituted C₁-C₆ alkyl-C₂-C₉ heteroaryl or optionally substituted C₁-C₆ alkyl-C₂-C₉ heterocyclyl; and

each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl.

[16] The compound, or a pharmaceutically acceptable salt thereof, of paragraph [13], wherein the K-Ras binding moiety is the structure of Formula III-2:

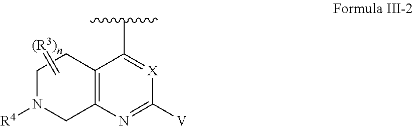

wherein n is 0, 1, 2, or 3;

X is N or CR′, wherein R′ is hydrogen, or R′ is attached to the linker via an optionally substituted C₁-C₃ alkylene bridge or optionally substituted C₁-C₃ heteroalkylene bridge;

V is CHR⁵, CR⁵R⁵, OR⁵, NHR⁵, or NR^{5a}R^{5b};

each R³ is

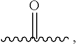 [SMILES: *=O]

R⁴ is optionally substituted C₆-C₁₀ aryl or optionally substituted C₂-C₉ heteroaryl;

each R⁵ is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl; and

each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl, or

R^{5a} and R^{5b}, together with the nitrogen atom to which each is attached, combine to form optionally substituted C₂-C₉ heterocyclyl;

provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge.

[17] The compound, or a pharmaceutically acceptable salt thereof, of paragraph [13], wherein the K-Ras binding moiety is the structure of Formula III-3:

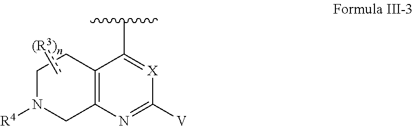

wherein n is 0, 1, 2, 3, 4, 5, or 6;

[symbol] represents a single bond or a double bond;

X is N or CR′, wherein R′ is hydrogen, or R′ is attached to the linker via an optionally substituted C₁-C₃ alkylene bridge or optionally substituted C₁-C₃ heteroalkylene bridge;

V is NR^{5a}R^{5b};

each R³ is, independently,

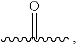 [SMILES: *=O]

optionally substituted C₁-C₆ alkyl, or optionally substituted C₁-C₆ heteroalkyl, or

R³ is attached to the linker via an optionally substituted C₁-C₃ alkylene bridge or optionally substituted C₁-C₃ heteroalkylene bridge;

R⁴ is optionally substituted C₆-C₁₀ aryl or optionally substituted C₂-C₉ heteroaryl;

R^{5a} and R^{5b}, together with the nitrogen atom to which each is attached, combine to form optionally substituted C₂-C₉ heterocyclyl;

provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge, and

further provided that when R³ is attached to the linker via an optionally substituted C₁-C₃ alkylene bridge or optionally substituted C₁-C₃ heteroalkylene bridge, R′ not is attached to the linker via an optionally substituted C₁-C₃ alkylene bridge or optionally substituted C₁-C₃ heteroalkylene bridge.

[18] The compound, or a pharmaceutically acceptable salt thereof, of any one of paragraphs [13] to [17], wherein R⁴ is

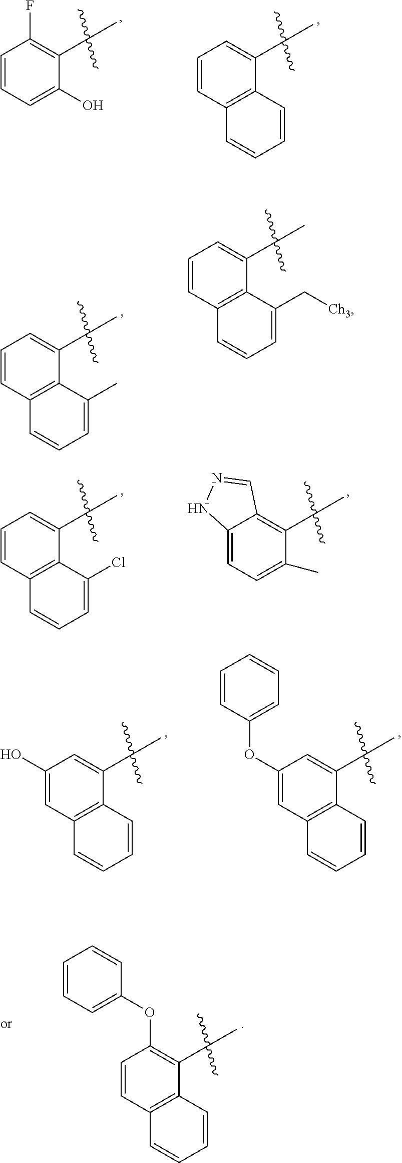

[19] The compound, or a pharmaceutically acceptable salt thereof, of any one of paragraphs [13] to [17], wherein R⁴ is

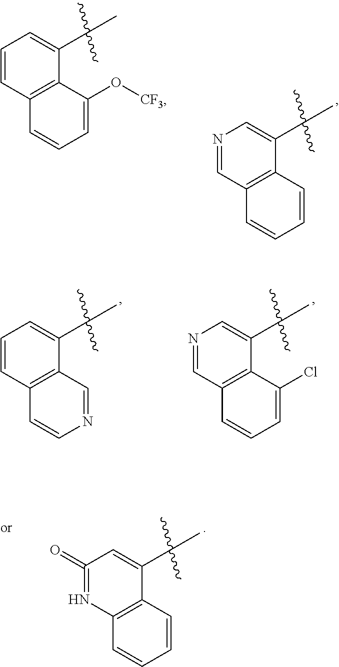

[20] The compound, or a pharmaceutically acceptable salt thereof, of any one of paragraphs [13] to [19], wherein V is

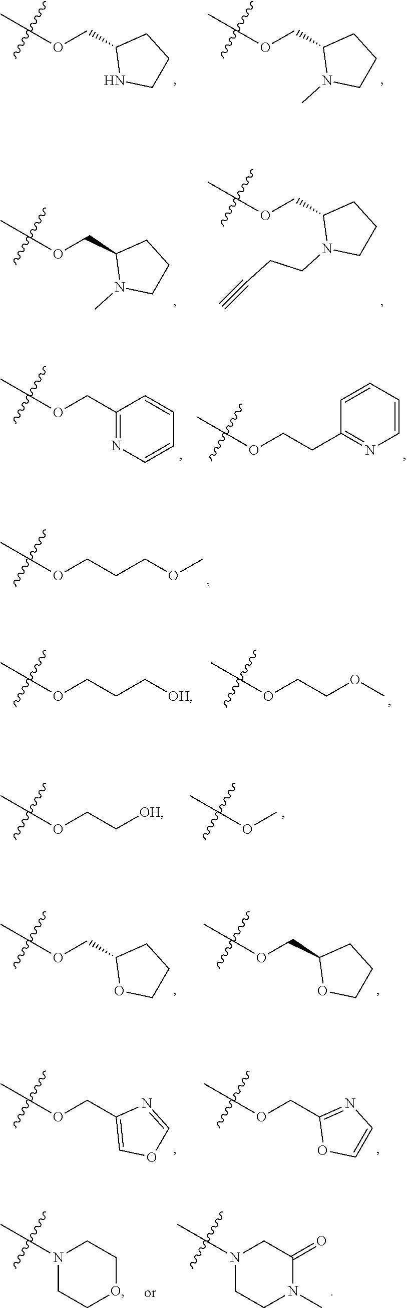

[21] The compound, or a pharmaceutically acceptable salt thereof, of any one of paragraphs [13] to [18], wherein Formula III has this structure:

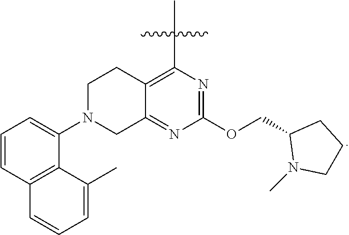 [SMILES: *c1nc(OC[C@@H]2CCCN2C)nc2c1CCN(c1cccc3cccc(C)c13)C2]

[22] The compound, or a pharmaceutically acceptable salt thereof, of paragraph [8], wherein the K-Ras binding moiety is the structure of Formula IV:

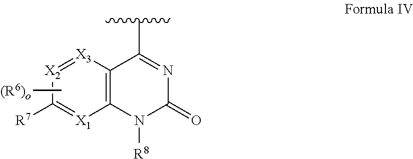

wherein o is 0, 1, or 2;

X¹, X² and X³ are each independently N, CH, or CR⁶;

each R⁶ is, independently, halo, CN, hydroxy, optionally substituted C₁-C₆ alkyl, or optionally substituted C₁-C₆ heteroalkyl, or

R⁶ is attached to the linker via a C₁-C₃ alkyl bridge or C₁-C₃ heteroalkyl bridge; and

R⁷ and R⁸ are, independently, optionally substituted C₆-C₁₀ aryl or optionally substituted C₂-C₉ heteroaryl.

[23] The compound, or a pharmaceutically acceptable salt thereof, of paragraph [22], wherein only one of X¹, X² and X³ is N.

[24] The compound, or a pharmaceutically acceptable salt thereof, of paragraph [22] or [23], wherein Formula IV has the structure:

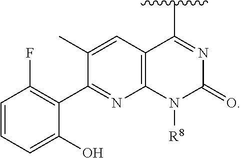

[25] The compound, or a pharmaceutically acceptable salt thereof, of paragraph [8], wherein the K-Ras binding moiety is the structure of Formula V:

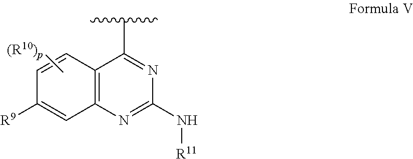

wherein p is 0, 1, 2, or 3;

R⁹ is optionally substituted C₆-C₁₀ aryl or optionally substituted C₂-C₉ heteroaryl;

each R¹⁰ is, independently, halo, CN, hydroxy, optionally substituted C₁-C₆ alkyl, or optionally substituted C₁-C₆ heteroalkyl, or R¹⁰ is attached to the linker via a C₁-C₃ alkylene bridge or C₁-C₃ heteroalkylene bridge; and

R¹¹ is optionally substituted C₂-C₉ heteroaryl or optionally substituted C₂-C₉ heterocyclyl.

[26] The compound, or a pharmaceutically acceptable salt thereof, of any one of paragraphs [1] to [25], wherein the linker positions a reactive atom of B about 5 to about 11 angstroms from the nearest atom of A.

[27] The compound, or a pharmaceutically acceptable salt thereof, of any one of paragraphs 1 to 29, wherein the linker positions a reactive atom of B 4 to 9 atoms from the nearest atom of A.

[28] The compound, or a pharmaceutically acceptable salt thereof, of any one of paragraphs [1] to [27], wherein the linker is the structure of Formula VI:

A¹-(B¹)ₐ-(C¹)_{b}-(B²)_{c}-(D)-(B³)_{d}-(C²)ₑ-(B⁴)_{f}-A² Formula VI

wherein A¹ is a bond between the linker and the Ras binding moiety;

A² is a bond between the selective cross-linking group and the linker;

B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;

C¹ and C² are each, independently, selected from carbonyl, thiocarbonyl, sulphonyl, or phosphoryl;

a, b, c, d, e, and f are each, independently, 0 or 1; and

D is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₂₋₆ heteroarylene, optionally substituted C₃₋₈ cycloalkylene, optionally substituted C₆₋₁₂ arylene, optionally substituted C₂-C₁₀ polyethylene glycol, or optionally substituted C₁₋₁₀ heteroalkylene, or a chemical bond linking A¹-(B¹)ₐ-(C¹)_{b}-(B²)_{c}- to -(B³)_{d}-(C²)ₑ-(B⁴)_{f}-A².

[29] The compound, or a pharmaceutically acceptable salt thereof, of any one of paragraphs [1] to [28], wherein the linker comprises a 3 to 8-membered heterocyclyl group.

[30] The compound, or a pharmaceutically acceptable salt thereof, of paragraph [29], wherein A-L-B is the structure of Formula VIIa or VIIb:

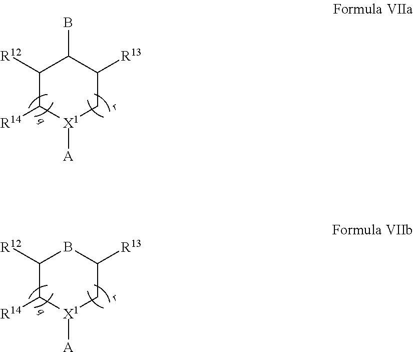

wherein q and r are, independently, 0, 1, or 2;

X¹ is N or CH;

R¹² and R¹³ are, independently, hydrogen, optionally substituted C₁-C₆ alkyl or optionally substituted C₁-C₆ heteroalkyl; and

R¹⁴ is hydrogen, optionally substituted C₁-C₆ alkyl, or optionally substituted C₁-C₆ heteroalkyl, wherein R¹⁴ optionally comprises a bond to A.

[31] The compound, or a pharmaceutically acceptable salt thereof, of paragraph [30], wherein A-L-B is selected from the group consisting of:

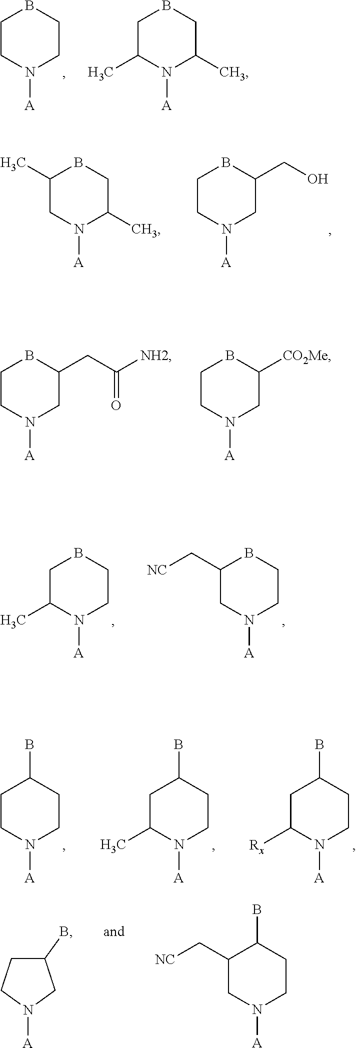

wherein Rₓ is an optionally substituted C₁-C₃ alkylene bridge or optionally substituted C₁-C₃ heteroalkylene bridge joined to A.

[32] The compound, or a pharmaceutically acceptable salt thereof, of paragraph [1], wherein -L-B is selected from the group consisting of:

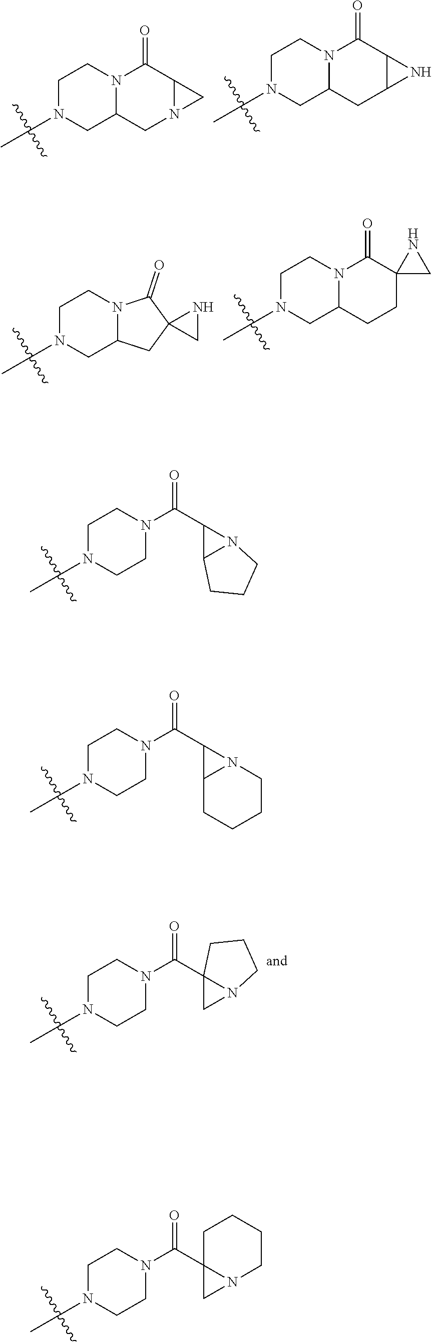

[33] The compound, or a pharmaceutically acceptable salt thereof, of paragraph [31], wherein A-L-B is:

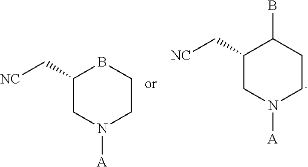

[34] The compound, or a pharmaceutically acceptable salt thereof, of paragraph [29], wherein A-L-B is the structure of Formula VIIc or Formula VIId:

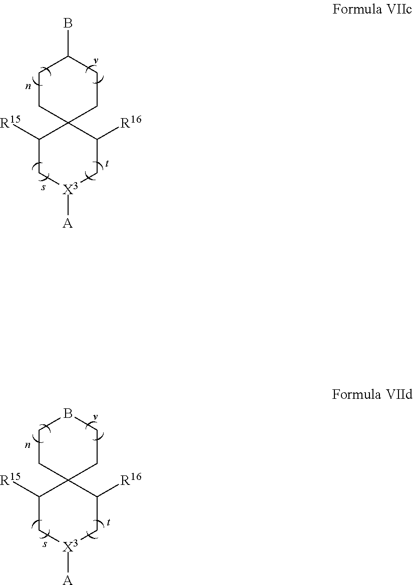

wherein s, t, u, and v are, independently, 0, 1, or 2;

X³ is N or CH; and

R¹⁵ and R¹⁶ are, independently, hydrogen, optionally substituted C₁-C₆ alkyl or optionally substituted C₁-C₆ heteroalkyl.

[35] The compound, or a pharmaceutically acceptable salt thereof, of paragraph [34], wherein A-L-B is:

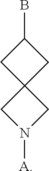

[36] The compound, or a pharmaceutically acceptable salt thereof, of any one of paragraphs [1] to [28], wherein the linker is acyclic.

[37] The compound, or a pharmaceutically acceptable salt thereof, of paragraph [36], wherein the linker is the structure of Formula VIII:

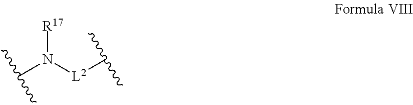

wherein R¹⁷ is hydrogen or optionally substituted C₁-C₆ alkyl; and

L² is optionally substituted C₁-C₄ alkylene or optionally substituted C₃-C₆ cycloalkylene.

[38] The compound, or a pharmaceutically acceptable salt thereof, of paragraph [37], wherein the linker is selected from the group consisting of:

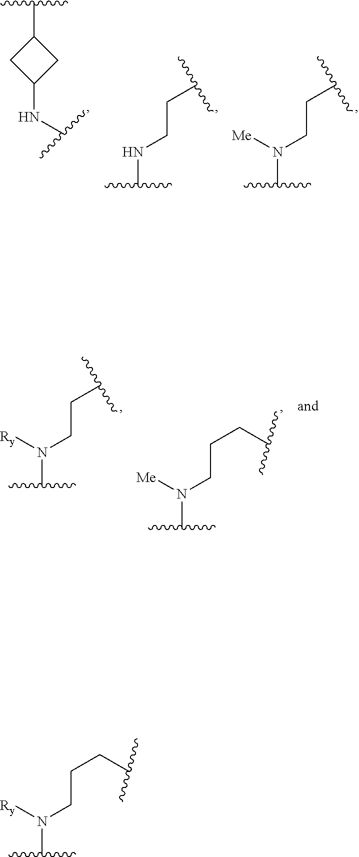

wherein R_{y} is an optionally substituted C₁-C₃ alkylene bridge or optionally substituted C₁-C₃ heteroalkylene bridge joined to A.

[39] The compound, or a pharmaceutically acceptable salt thereof, of any one of paragraphs [1] to [38], wherein the selective cross-linking group is a C—O bond forming selective cross-linking group.

[40] The compound, or a pharmaceutically acceptable salt thereof, of any one of paragraphs [1] to [39], wherein the selective cross-linking group comprises a carbodiimide, an aminooxazoline, a chloroethyl urea, an aziridine, a trifluoromethyl ketone, a boronic acid, a boronic ester, an N-ethoxycarbonyl-2-ethoxy-1,2-dihydroquinoline (EEDQ), an iso-EEDQ or other EEDQ derivative, an epoxide, an oxazolium, or a glycal.

[41] The compound, or a pharmaceutically acceptable salt thereof, of any one of paragraphs [1] to [40], wherein the selective cross-linking group is the structure of Formula IX:

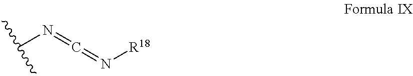

wherein R¹⁸ is optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl.

[42] The compound, or a pharmaceutically acceptable salt thereof, of paragraph [31], wherein the selective cross-linking group is selected from the group consisting of:

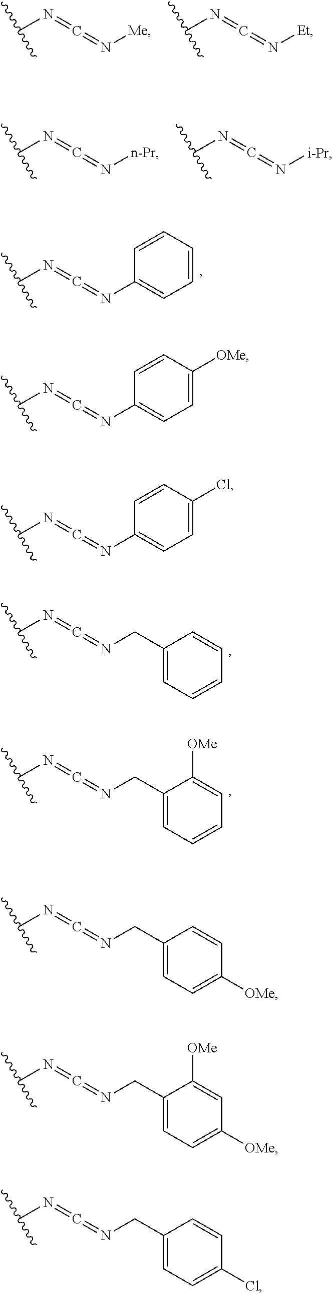

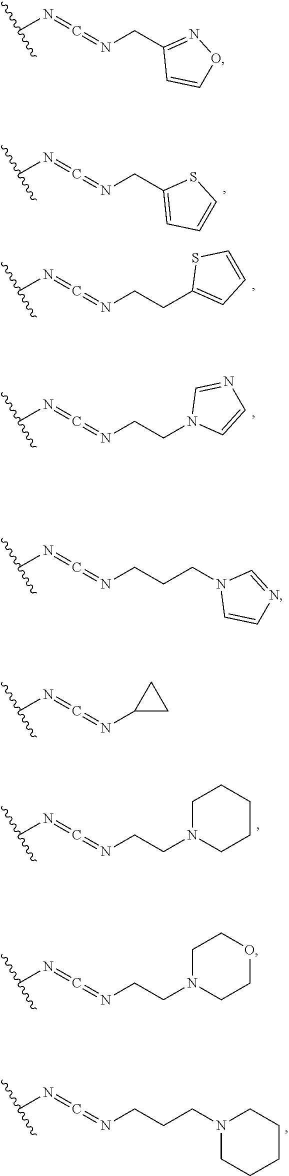

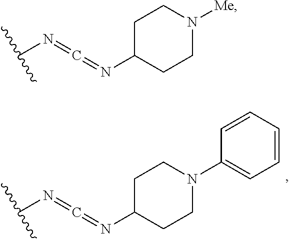 [SMILES: *N=C=NC1CCN(C)CC1 ; *N=C=NC1CCN(c2ccccc2)CC1]

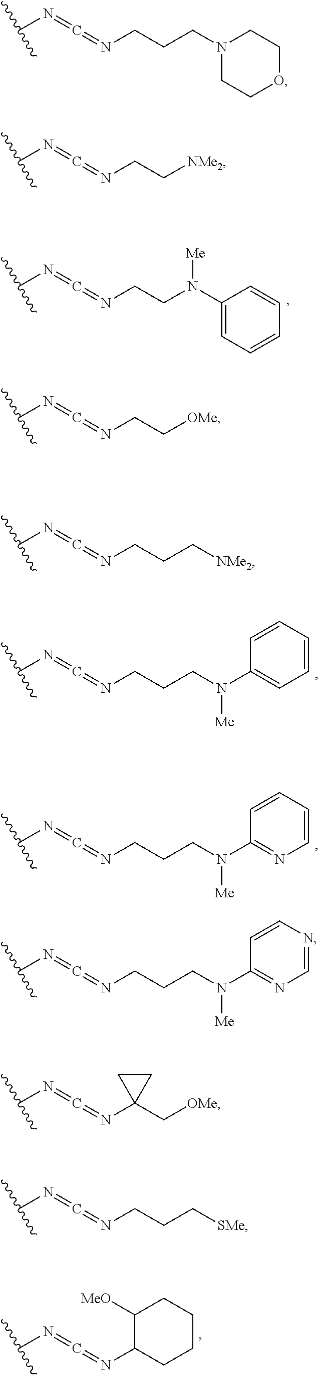

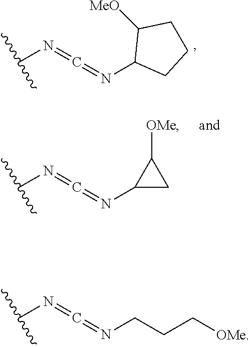 [SMILES: *N=C=NC1CCCC1OC ; *N=C=NC1CC1OC ; *N=C=NCCCOC]

[43] The compound, or a pharmaceutically acceptable salt thereof, of any one of paragraphs [1] to [40], wherein the selective cross-linking group is the structure of Formula Xa or Xb:

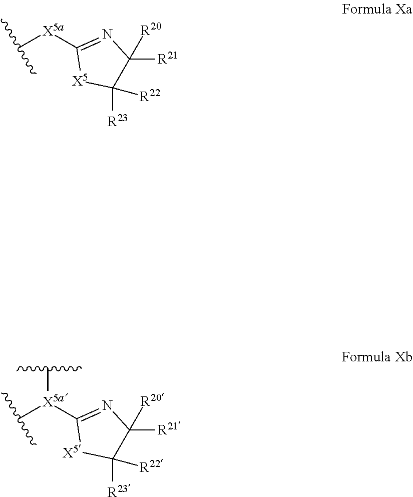

wherein X⁵ is O or S;

X^{5′} is O or S;

X^{5a} is absent or NR¹⁹;

X^{5a′} is N, wherein said N is a ring atom of an optionally substituted C₂-C₉ heterocyclyl group;

R¹⁹ is hydrogen, optionally substituted C₁-C₆ alkyl, optionally substituted C₆-C₁₀ aryl, optionally substituted C₂-C₉ heterocyclyl, or optionally substituted C₂-C₉ heteroaryl; and

R²⁰, R²¹, R²², R²³, R^{20′}, R^{21′}, R^{22′}, and R^{23′} are, independently, hydrogen or optionally substituted C₁-C₆ alkyl.

[44] The compound, or a pharmaceutically acceptable salt thereof, of paragraph [43], wherein the selective cross-linking group is selected from the group consisting of:

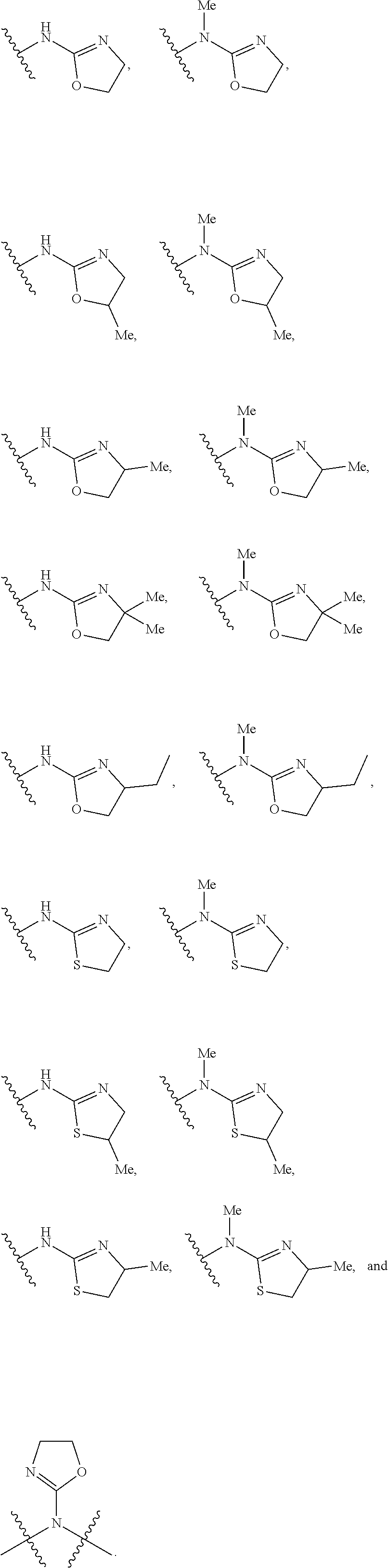

[45] The compound, or a pharmaceutically acceptable salt thereof, of any one of paragraphs [1] to [40], wherein the selective cross-linking group is the structure of Formula XIa or Formula XIb:

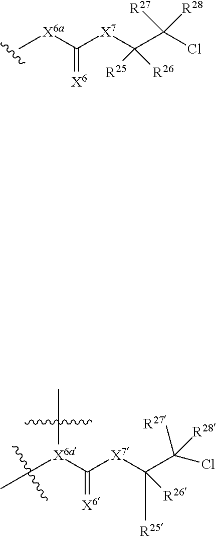

wherein X⁶ is O or S;

X^{6′} is O or S;

X^{6a} is absent or NR²⁴;

X^{6a′} is N, wherein said N is a ring atom of an optionally substituted C₂-C₉ heterocyclyl group;

X⁷ and X^{7′} are each O, S, or NR²⁹;

R²⁴ is hydrogen, optionally substituted C₁-C₆ alkyl, optionally substituted C₆-C₁₀ aryl, optionally substituted C₂-C₉ heterocyclyl, or optionally substituted C₂-C₉ heteroaryl; and

R²⁵, R²⁶, R²⁷, R²⁸, R²⁹, R^{25′}, R^{26′}, R^{27′}, and R^{28′}, are, independently, hydrogen or optionally substituted C₁-C₆ alkyl.

[46] The compound, or a pharmaceutically acceptable salt thereof, of paragraph [45], wherein the selective cross-linking group is selected from the group consisting of:

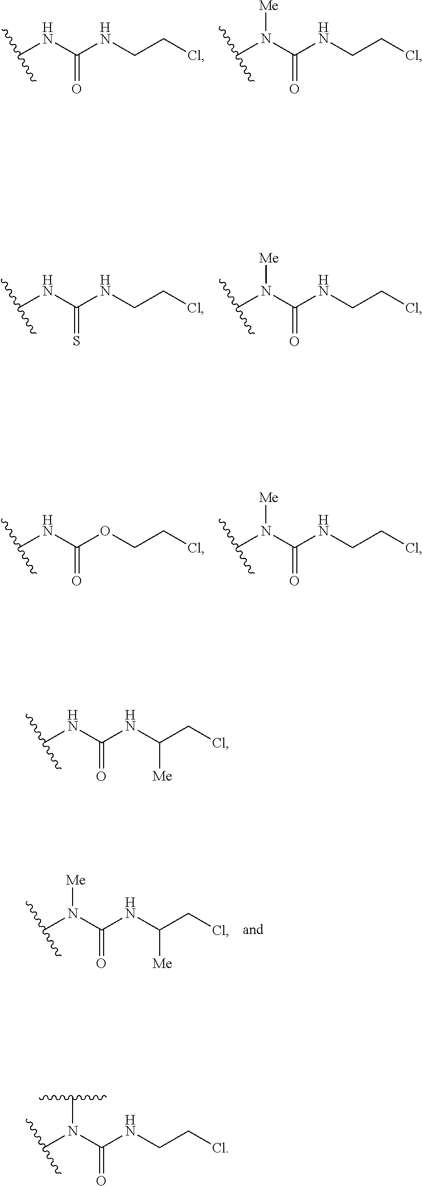

[47] The compound, or a pharmaceutically acceptable salt thereof, of any one of paragraphs [1] to [40], wherein the selective cross-linking group is the structure of Formula XIIa, XIIb, XIIc, XIId or XIIe:

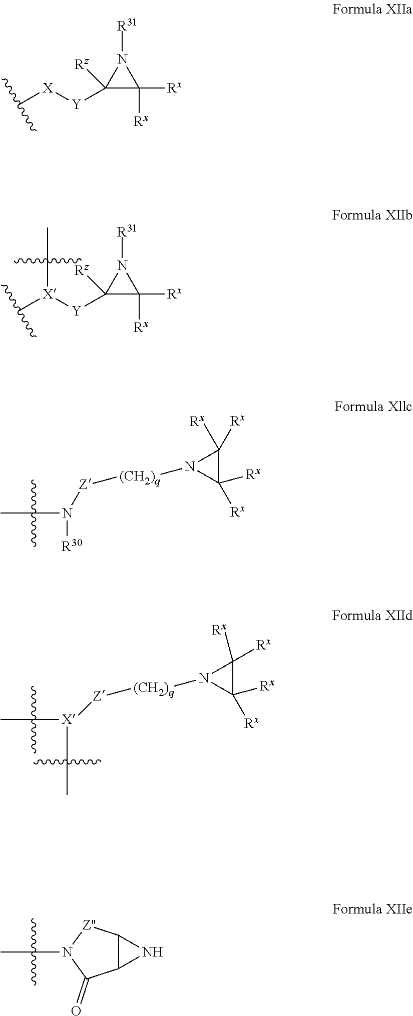

wherein X is absent or NR³⁰;

X′ is N, wherein said N is a ring atom of an optionally substituted C₂-C₉ heterocyclyl group;

Y is C(O), C(S), SO₂, or optionally substituted C₁-C₃ alkyl;

Z′ is C(O) or SO₂;

Z″ is —CH₂— or C(O);

q is 0, 1, or 2;

each R^{x} is, independently, hydrogen, CN, C(O)R^{y}, CO₂R^{y}, C(O)NR^{y}R^{y} optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl;

each R^{y} is, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl;

R^{z} is hydrogen or CH₃;

R³⁰ is hydrogen or optionally substituted C₁-C₆ alkyl;

R³¹ is hydrogen, —C(O)R³², —SO₂R³³, optionally substituted C₁-C₆ alkyl, optionally substituted C₁-C₆ heteroalkyl, optionally substituted C₆-C₁₀ aryl, optionally substituted C₂-C₉ heterocyclyl, or optionally substituted C₂-C₉ heteroaryl; and

R³² and R³³ are, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl.

[48] The compound, or a pharmaceutically acceptable salt thereof, of paragraph [47], wherein at least two of R³¹ and R^{x} is hydrogen.

[49] The compound, or a pharmaceutically acceptable salt thereof, of paragraph [47], wherein R³¹ is CH₃, C(O)CH₃, SO₂CH₃, CH₂—C₆H₅, or CH₂CH₂OCH₃.

[50] The compound, or a pharmaceutically acceptable salt thereof, of paragraph [47], wherein the selective cross-linking group is selected from the group consisting of:

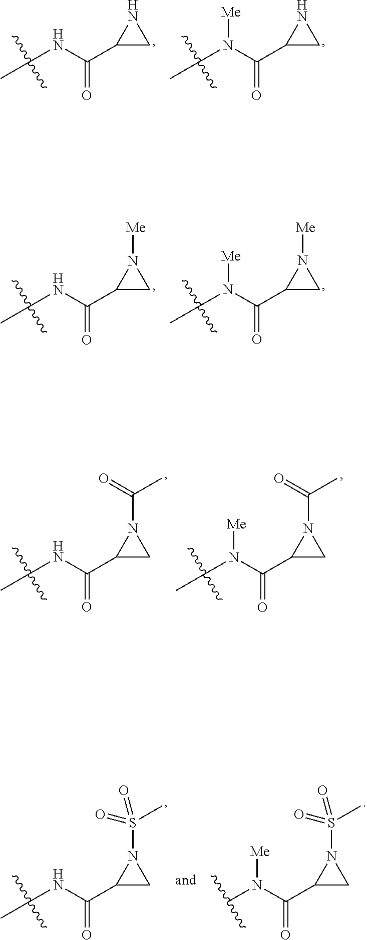

[51] The compound, or a pharmaceutically acceptable salt thereof, of paragraph [40], wherein the selective cross-linking group is selected from the group consisting of:

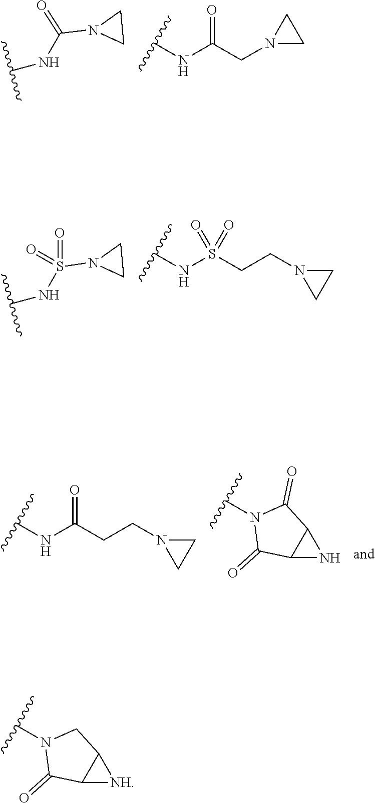

[52] The compound, or a pharmaceutically acceptable salt thereof, of paragraph [40], wherein the selective cross-linking group is selected from the group consisting of:

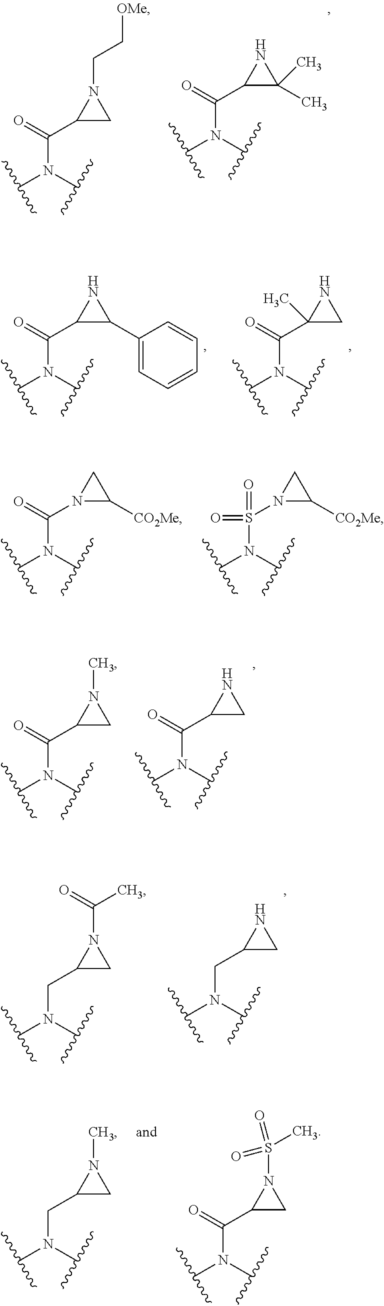

[53] The compound, or a pharmaceutically acceptable salt thereof, of paragraph [40], wherein the selective cross-linking group is selected from the group consisting of:

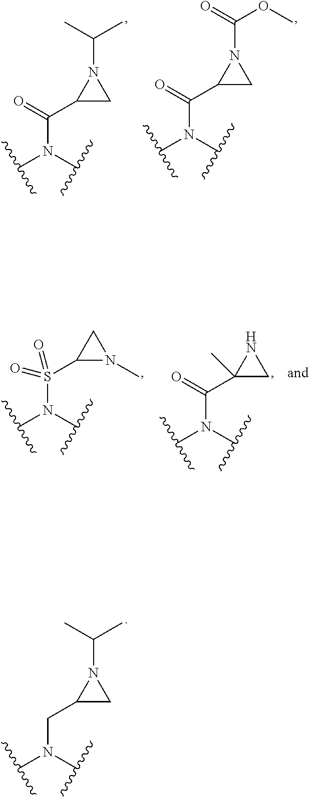

[54] The compound, or a pharmaceutically acceptable salt thereof, of paragraph [40], selected from:

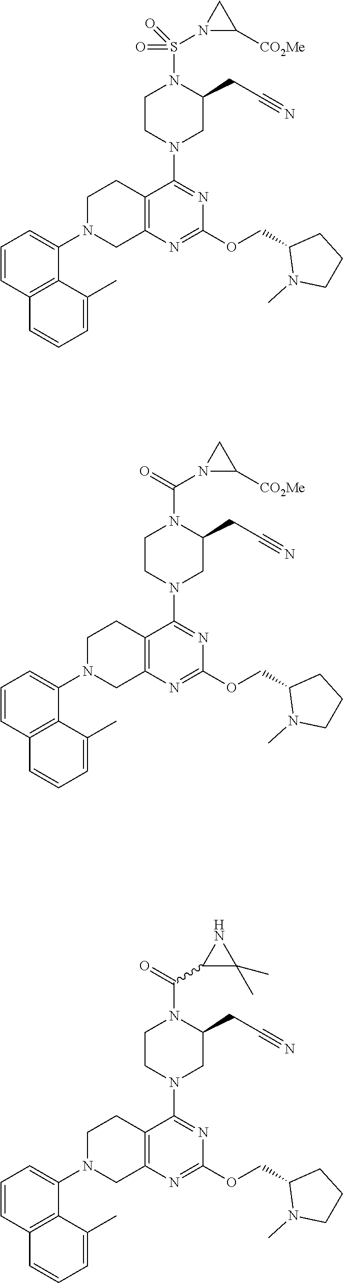 [SMILES: COC(=O)C1CN1S(=O)(=O)N1CCN(c2nc(OC[C@@H]3CCCN3C)nc3c2CCN(c2cccc4cccc(C)c24)C3)C[C@@H]1CC#N ; COC(=O)C1CN1C(=O)N1CCN(c2nc(OC[C@@H]3CCCN3C)nc3c2CCN(c2cccc4cccc(C)c24)C3)C[C@@H]1CC#N ; Cc1cccc2cccc(N3CCc4c(nc(OC[C@@H]5CCCN5C)nc4N4CCN(C(=O)C5NC5(C)C)[C@@H](CC#N)C4)C3)c12]

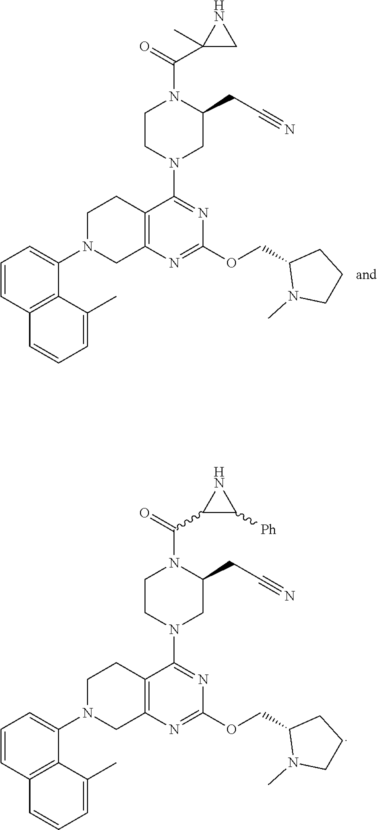 [SMILES: Cc1cccc2cccc(N3CCc4c(nc(OC[C@@H]5CCCN5C)nc4N4CCN(C(=O)C5(C)CN5)[C@@H](CC#N)C4)C3)c12 ; Cc1cccc2cccc(N3CCc4c(nc(OC[C@@H]5CCCN5C)nc4N4CCN(C(=O)C5NC5c5ccccc5)[C@@H](CC#N)C4)C3)c12]

[55] The compound, or a pharmaceutically acceptable salt thereof, of paragraph [40], having the structure of Formula XXIV:

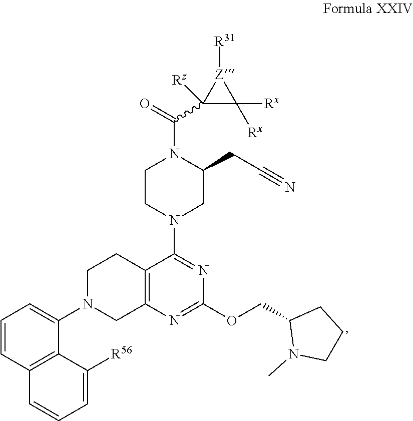

wherein R³¹ is absent, hydrogen, C(O)CH₃, SO₂CH₃, optionally substituted C₁-C₆ alkyl, optionally substituted C₁-C₆ heteroalkyl, optionally substituted C₆-C₁₀ aryl, optionally substituted C₁-C₃ alkyl-C₆-C₁₀ aryl, optionally substituted C₂-C₉ heterocyclyl, or optionally substituted C₁-C₃ alkyl-C₂-C₉ heterocyclyl;

R⁵⁶ is CH₃ or Cl;

R^{z} is hydrogen, optionally substituted C₁-C₃ alkyl;
each R^{x} is, independently, hydrogen, CO₂CH₃, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, optionally substituted C₂-C₆ alkenyl, or optionally substituted C₂-C₆ alkynyl; and

Z′″ is N or O.

[56] The compound, or a pharmaceutically acceptable salt thereof, of paragraph [40] or [55], having the structure of Formula XIII:

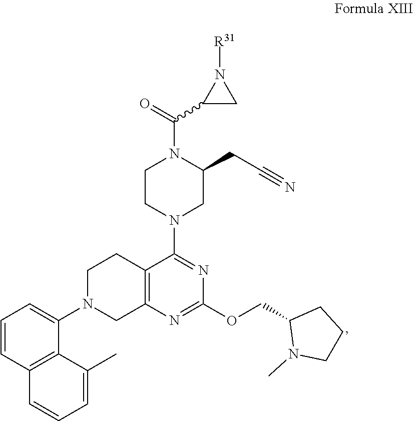

wherein R³¹ is hydrogen, CH₃, C(O)CH₃, SO₂CH₃, CH₂—C₆H₅, or CH₂CH₂OCH₃.

[57] The compound, or a pharmaceutically acceptable salt thereof, of paragraph [40] or [55], having the structure of Formula XXV:

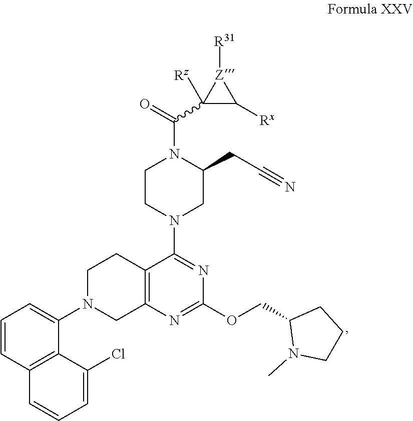

wherein R³¹ is absent, hydrogen, C(O)CH₃, optionally substituted C₁-C₆ alkyl, optionally substituted C₁-C₆ heteroalkyl, optionally substituted C₆-C₁₀ aryl, optionally substituted C₁-C₃ alkyl-C₆-C₁₀ aryl, optionally substituted C₂-C₉ heterocyclyl, or optionally substituted C₁-C₃ alkyl-C₂-C₉ heterocyclyl;

R^{z} is hydrogen, optionally substituted C₁-C₃ alkyl;

R^{x} is hydrogen, CO₂CH₃, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, optionally substituted C₂-C₆ alkenyl, or optionally substituted C₂-C₆ alkynyl; and

Z′″ is N or O.

[58] The compound, or a pharmaceutically acceptable salt thereof, of any one of paragraphs [1] to [40], wherein the selective cross-linking group is:

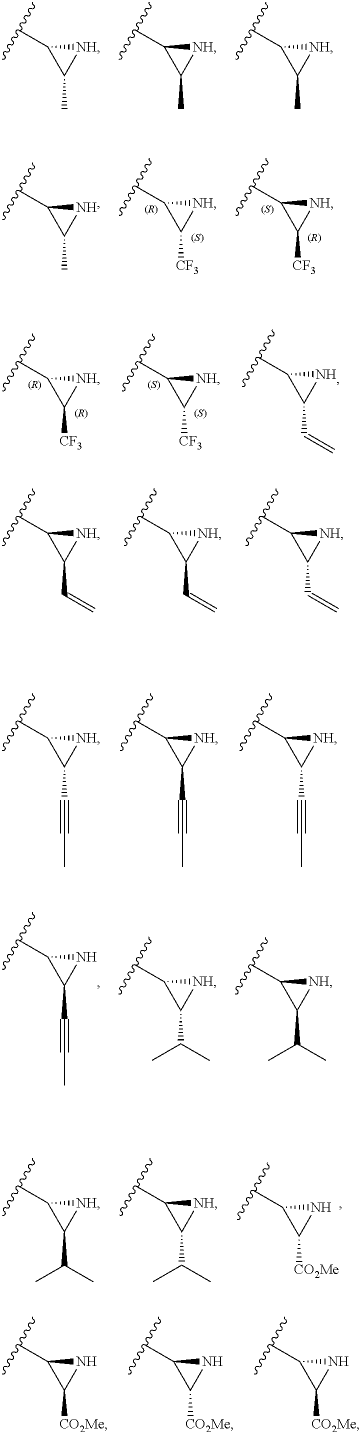

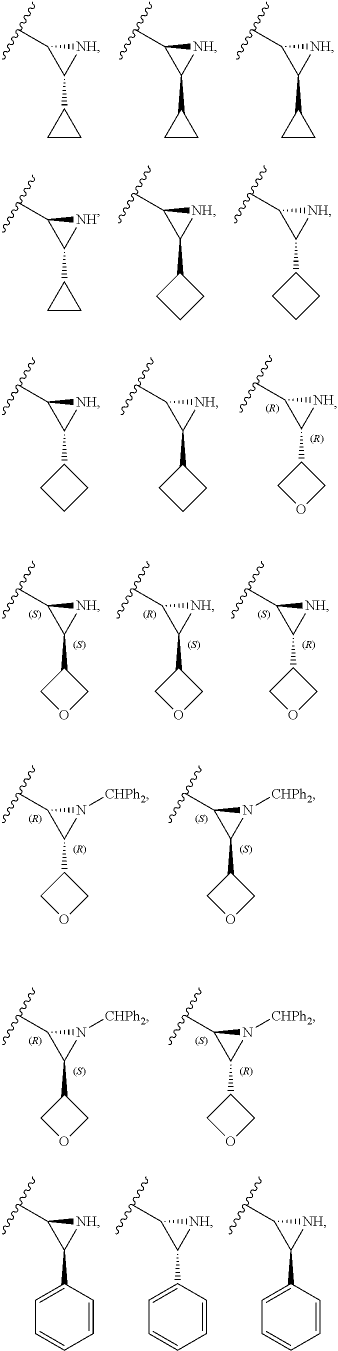

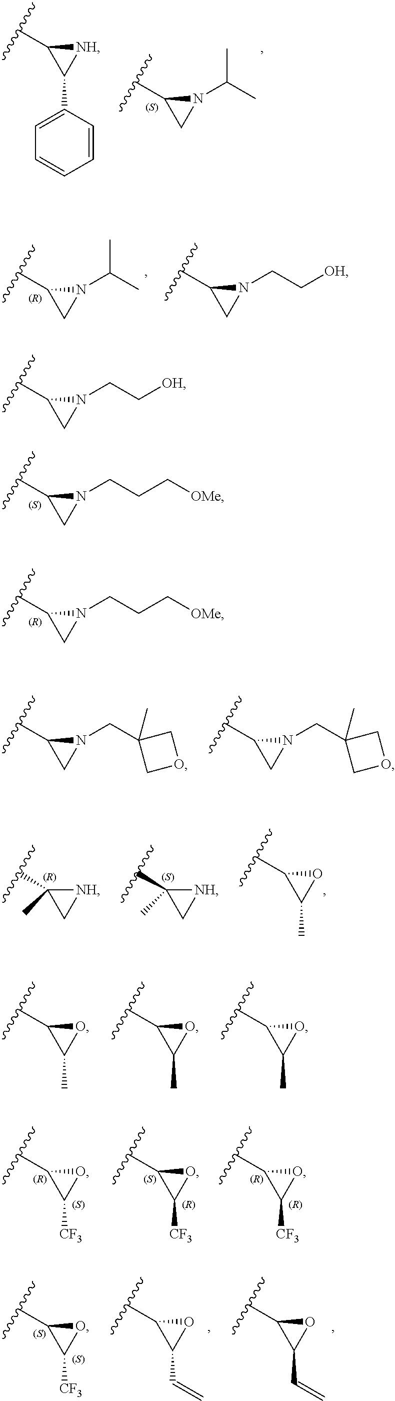

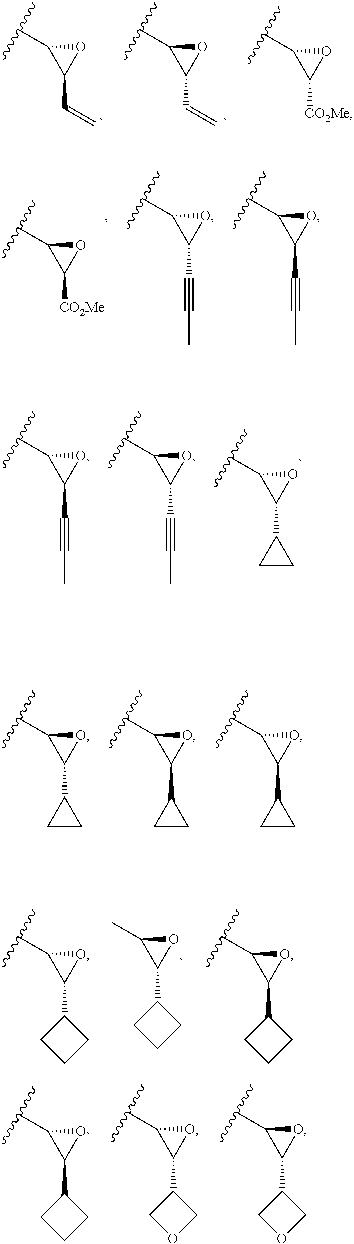

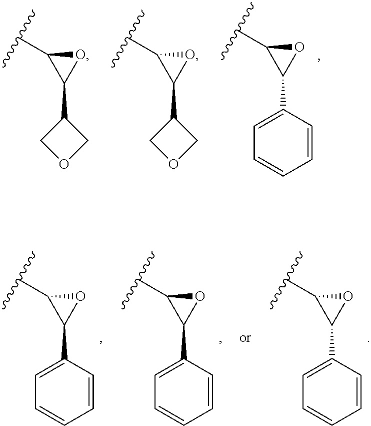 [SMILES: *[C@H]1O[C@H]1C1COC1 ; *[C@@H]1O[C@H]1C1COC1 ; *[C@H]1O[C@@H]1c1ccccc1 ; *[C@@H]1O[C@H]1c1ccccc1 ; *[C@H]1O[C@H]1c1ccccc1 ; *[C@@H]1O[C@@H]1c1ccccc1]

[59] The compound, or a pharmaceutically acceptable salt thereof, of any one of paragraphs [1] to [40] wherein the selective cross-linking group is:

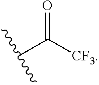 [SMILES: *C(=O)C(F)(F)F]

[60] The compound, or a pharmaceutically acceptable salt thereof, of any one of paragraphs [1] to [40], wherein the selective cross-linking is:

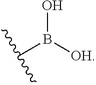 [SMILES: *B(O)O]

[61] The compound, or a pharmaceutically acceptable salt thereof, of any one of paragraphs [1] to [40], wherein the selective cross-linking group is the structure of Formula XIV:

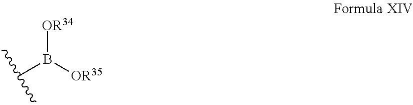

wherein R³⁴ and R³⁵ are, independently, optionally substituted C₁-C₆ alkyl, or R³⁴ and R³⁵ combine with the boron to which they are attached to form an optionally substituted heterocyclyl.

[62] The compound, or a pharmaceutically acceptable salt thereof, of any one of paragraphs [1] to [40], wherein the selective cross-linking group is the structure of Formula XV:

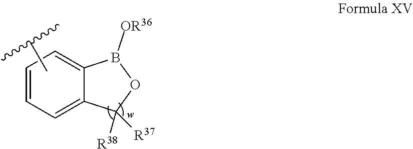

wherein w is 1 or 2;

R³⁶ is hydrogen or optionally substituted C₁-C₆ alkyl; and

each R³⁷ and R³⁸ is, independently, hydrogen or optionally substituted C₁-C₆ alkyl.

[63] The compound, or a pharmaceutically acceptable salt thereof, of paragraph [61] or [62], wherein the -selective cross-linking group is selected from the group consisting of:

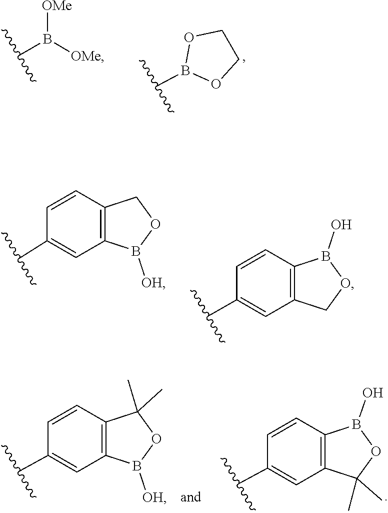

[64] The compound, or a pharmaceutically acceptable salt thereof, of any one of paragraphs [1] to [40], wherein the selective cross-linking group is the structure of Formula XVI:

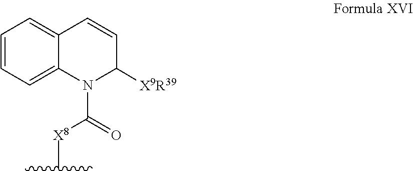

wherein X⁸ is absent, O, S, NR⁴⁰, or CH₂;

X⁹ is O, NR⁴¹, S, S(O), or S(O)₂;

R³⁹ is optionally substituted C₁-C₆ alkyl; and

R⁴⁰ and R⁴¹ are, independently, hydrogen or optionally substituted C₁-C₆ alkyl.

[65] The compound, or a pharmaceutically acceptable salt thereof, of paragraph [64], wherein the selective cross-linking group is:

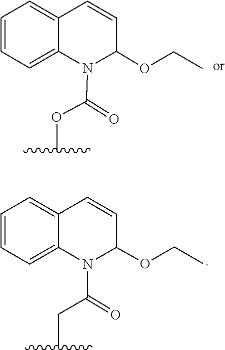

[66] The compound, or a pharmaceutically acceptable salt thereof, of any one of paragraphs [1] to [40], wherein the selective cross-linking group is the structure of Formula XVII:

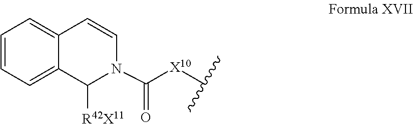

wherein X¹⁰ is absent, O, S, NR⁴³, or CH₂;

X¹¹ is O, NR⁴⁴, S, S(O), or S(O)₂;

R⁴² is optionally substituted C₁-C₆ alkyl; and

R⁴³ and R⁴⁴ are, independently, hydrogen or optionally substituted C₁-C₆ alkyl.

[67] The compound, or a pharmaceutically acceptable salt thereof, of paragraph [66], wherein the selective cross-linking group is:

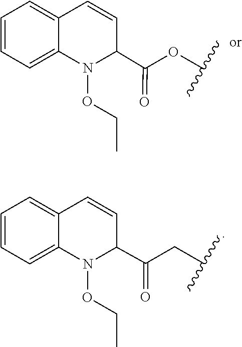 [SMILES: *OC(=O)C1C=Cc2ccccc2N1OCC ; *CC(=O)C1C=Cc2ccccc2N1OCC]

[68] The compound, or a pharmaceutically acceptable salt thereof, of any one of paragraphs [1] to [40], wherein the selective cross-linking group is the structure of Formula XVIII:

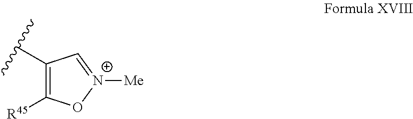

wherein R⁴⁵ is hydrogen or optionally substituted C₁-C₆ alkyl.

[69] The compound, or a pharmaceutically acceptable salt thereof, of paragraph [68], wherein the selective cross-linking group is:

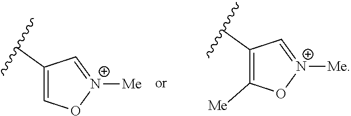 [SMILES: *c1co[n+](C)c1 ; *c1c[n+](C)oc1C]

[70] The compound, or a pharmaceutically acceptable salt thereof, of any one of paragraphs [1] to [40], wherein the selective cross-linking group is the structure of Formula XIX:

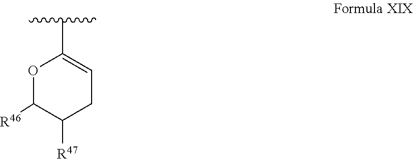

wherein R⁴⁶ and R⁴⁷ are, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl.

[71] The compound, or a pharmaceutically acceptable salt thereof, of paragraph [1], having the structure of Formula XX or XXI:

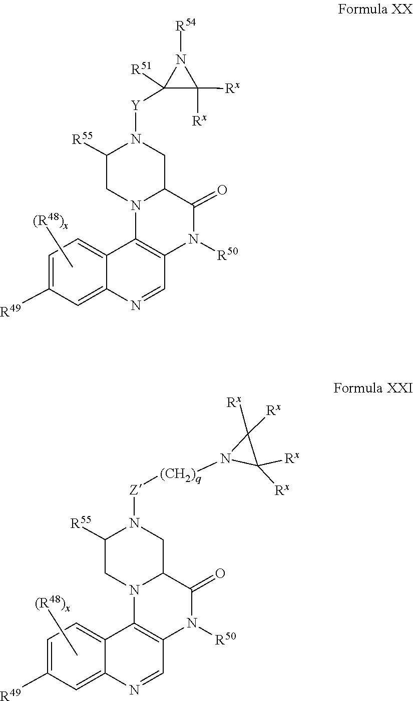

wherein Y is C(O), C(S), SO₂, or optionally substituted C₁-C₆ alkyl;

Z′ is C(O) or SO₂;

q is 0, 1 or 2;

x is0, 1, 2 or 3;

each R^{X} is, independently, hydrogen, CN, C(O)R^{y}, CO₂R^{y}, C(O)NR^{y}R^{y} optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl;

each R^{y} is, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl;

each R⁴⁸ is, independently, CN, halo, hydroxy, optionally substituted C₁-C₆ alkyl, or optionally substituted C₁-C₆ heteroalkyl, or

R⁴⁹ is optionally substituted C₆-C₁₀ aryl or optionally substituted C₂-C₉ heteroaryl;

R⁵⁰ is hydrogen or C₁-C₆ alkyl;

R⁵¹ is hydrogen, CN or C₁-C₆ alkyl;

R⁵⁴ is hydrogen, —C(O)R³², —SO₂R³³, optionally substituted C₁-C₆ alkyl, optionally substituted C₁-C₆ heteroalkyl, optionally substituted C₆-C₁₀ aryl, optionally substituted C₂-C₉ heterocyclyl, or optionally substituted C₂-C₉ heteroaryl; and

R⁵⁵ is hydrogen or optionally substituted C₁-C₆ alkyl.

[72] The compound, or a pharmaceutically acceptable salt thereof, of paragraph [71], where R⁵¹, R⁵⁴ and R^{x} are each hydrogen.

[73] The compound, or a pharmaceutically acceptable salt thereof, of paragraph [71] or [72] having the structure of Formula XXII or Formula XXIII:

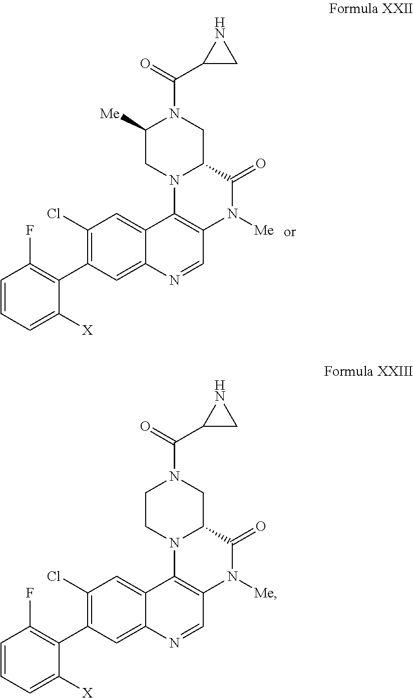

wherein X is hydrogen or hydroxy.

[74] A compound, or a pharmaceutically acceptable salt thereof, having the structure:

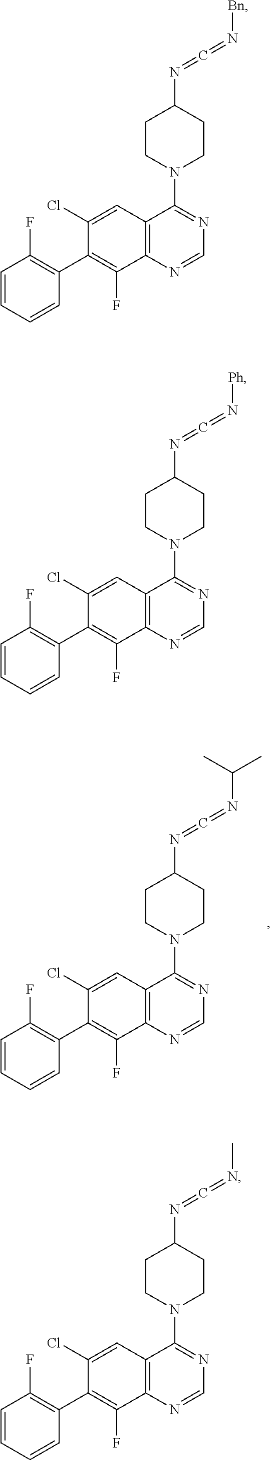 [SMILES: Fc1ccccc1-c1c(Cl)cc2c(N3CCC(N=C=NCc4ccccc4)CC3)ncnc2c1F ; Fc1ccccc1-c1c(Cl)cc2c(N3CCC(N=C=Nc4ccccc4)CC3)ncnc2c1F ; CC(C)N=C=NC1CCN(c2ncnc3c(F)c(-c4ccccc4F)c(Cl)cc23)CC1 ; CN=C=NC1CCN(c2ncnc3c(F)c(-c4ccccc4F)c(Cl)cc23)CC1]

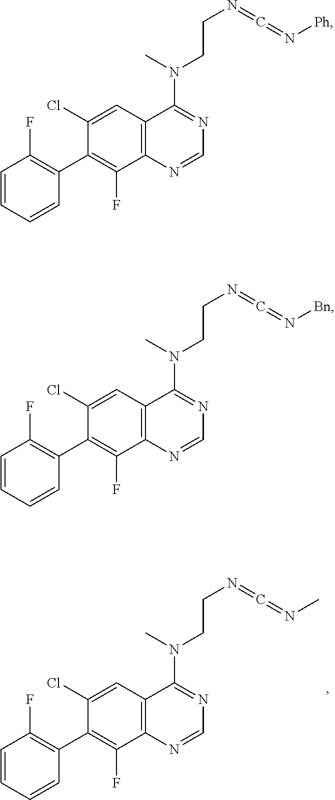 [SMILES: CN(CCN=C=Nc1ccccc1)c1ncnc2c(F)c(-c3ccccc3F)c(Cl)cc12 ; CN(CCN=C=NCc1ccccc1)c1ncnc2c(F)c(-c3ccccc3F)c(Cl)cc12 ; CN=C=NCCN(C)c1ncnc2c(F)c(-c3ccccc3F)c(Cl)cc12]

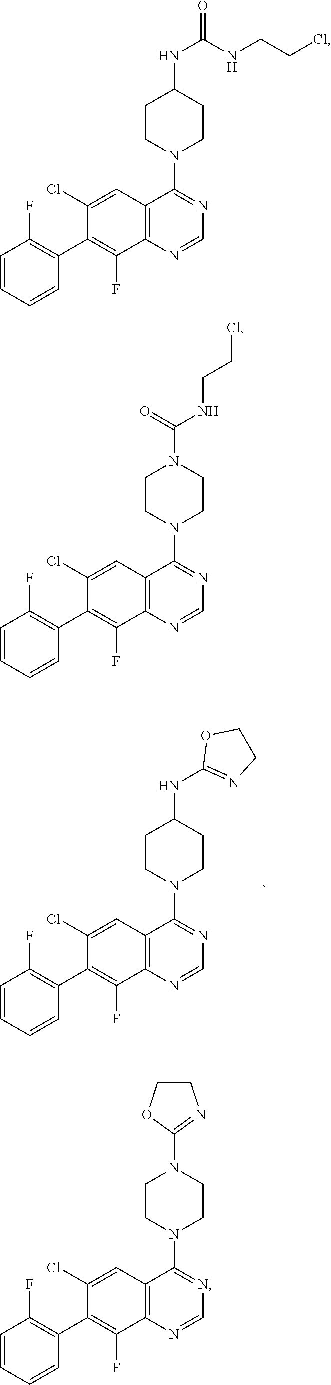 [SMILES: O=C(NCCCl)NC1CCN(c2ncnc3c(F)c(-c4ccccc4F)c(Cl)cc23)CC1 ; O=C(NCCCl)N1CCN(c2ncnc3c(F)c(-c4ccccc4F)c(Cl)cc23)CC1 ; Fc1ccccc1-c1c(Cl)cc2c(N3CCC(NC4=NCCO4)CC3)ncnc2c1F ; Fc1ccccc1-c1c(Cl)cc2c(N3CCN(C4=NCCO4)CC3)ncnc2c1F]

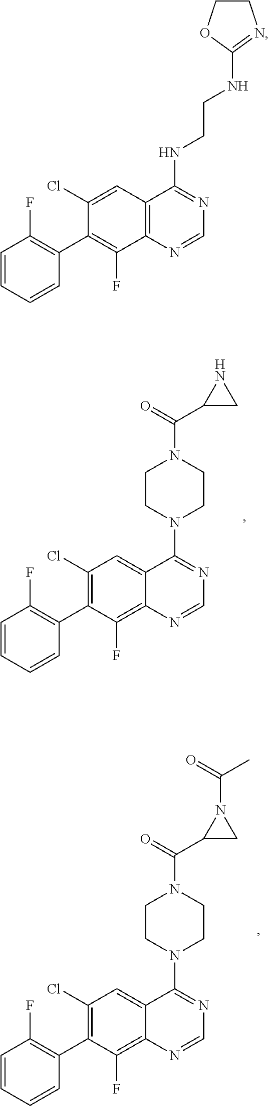 [SMILES: Fc1ccccc1-c1c(Cl)cc2c(NCCNC3=NCCO3)ncnc2c1F ; O=C(C1CN1)N1CCN(c2ncnc3c(F)c(-c4ccccc4F)c(Cl)cc23)CC1 ; CC(=O)N1CC1C(=O)N1CCN(c2ncnc3c(F)c(-c4ccccc4F)c(Cl)cc23)CC1]

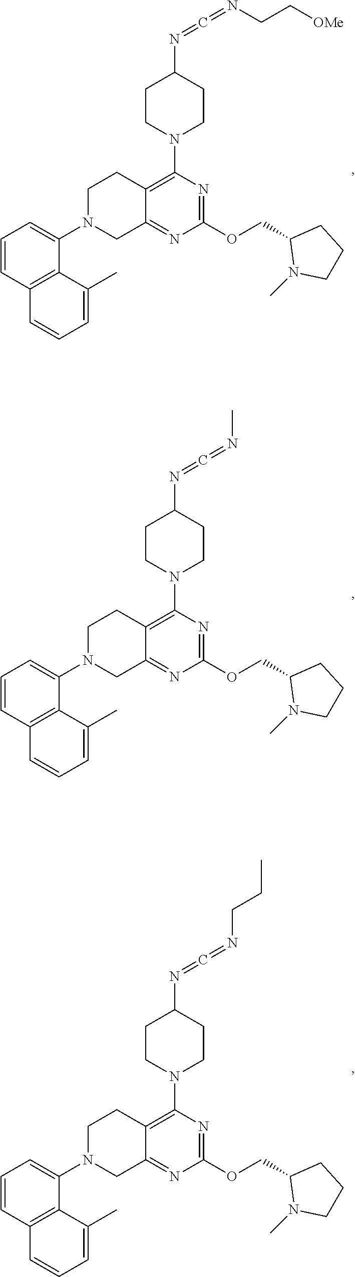 [SMILES: COCCN=C=NC1CCN(c2nc(OC[C@@H]3CCCN3C)nc3c2CCN(c2cccc4cccc(C)c24)C3)CC1 ; CN=C=NC1CCN(c2nc(OC[C@@H]3CCCN3C)nc3c2CCN(c2cccc4cccc(C)c24)C3)CC1 ; CCCN=C=NC1CCN(c2nc(OC[C@@H]3CCCN3C)nc3c2CCN(c2cccc4cccc(C)c24)C3)CC1]

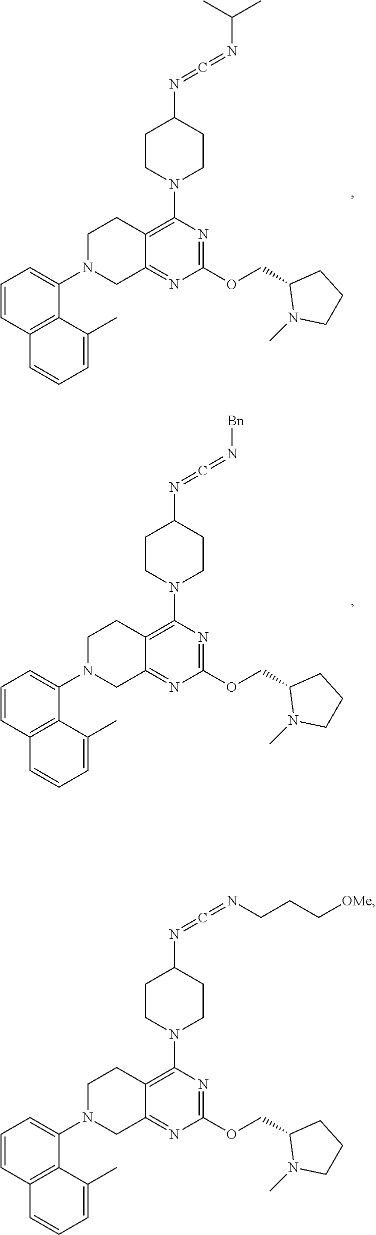 [SMILES: Cc1cccc2cccc(N3CCc4c(nc(OC[C@@H]5CCCN5C)nc4N4CCC(N=C=NC(C)C)CC4)C3)c12 ; Cc1cccc2cccc(N3CCc4c(nc(OC[C@@H]5CCCN5C)nc4N4CCC(N=C=NCc5ccccc5)CC4)C3)c12 ; COCCCN=C=NC1CCN(c2nc(OC[C@@H]3CCCN3C)nc3c2CCN(c2cccc4cccc(C)c24)C3)CC1]

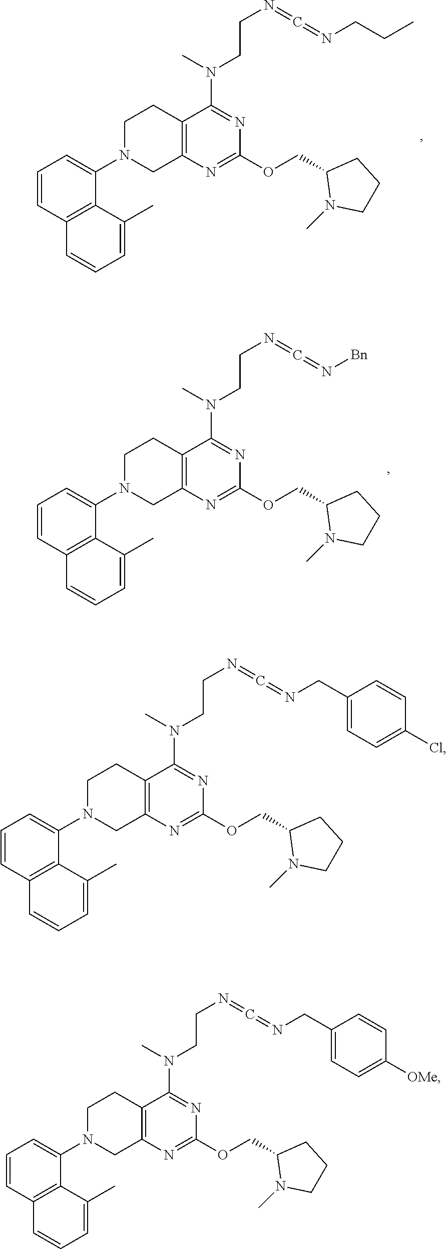 [SMILES: CCCN=C=NCCN(C)c1nc(OC[C@@H]2CCCN2C)nc2c1CCN(c1cccc3cccc(C)c13)C2 ; Cc1cccc2cccc(N3CCc4c(nc(OC[C@@H]5CCCN5C)nc4N(C)CCN=C=NCc4ccccc4)C3)c12 ; Cc1cccc2cccc(N3CCc4c(nc(OC[C@@H]5CCCN5C)nc4N(C)CCN=C=NCc4ccc(Cl)cc4)C3)c12 ; COc1ccc(CN=C=NCCN(C)c2nc(OC[C@@H]3CCCN3C)nc3c2CCN(c2cccc4cccc(C)c24)C3)cc1]

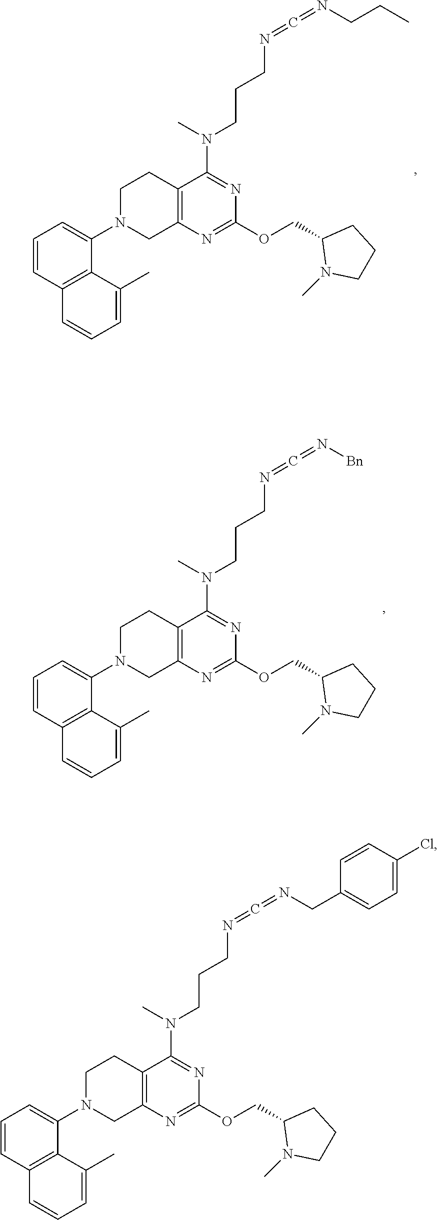 [SMILES: CCCN=C=NCCCN(C)c1nc(OC[C@@H]2CCCN2C)nc2c1CCN(c1cccc3cccc(C)c13)C2 ; Cc1cccc2cccc(N3CCc4c(nc(OC[C@@H]5CCCN5C)nc4N(C)CCCN=C=NCc4ccccc4)C3)c12 ; Cc1cccc2cccc(N3CCc4c(nc(OC[C@@H]5CCCN5C)nc4N(C)CCCN=C=NCc4ccc(Cl)cc4)C3)c12]

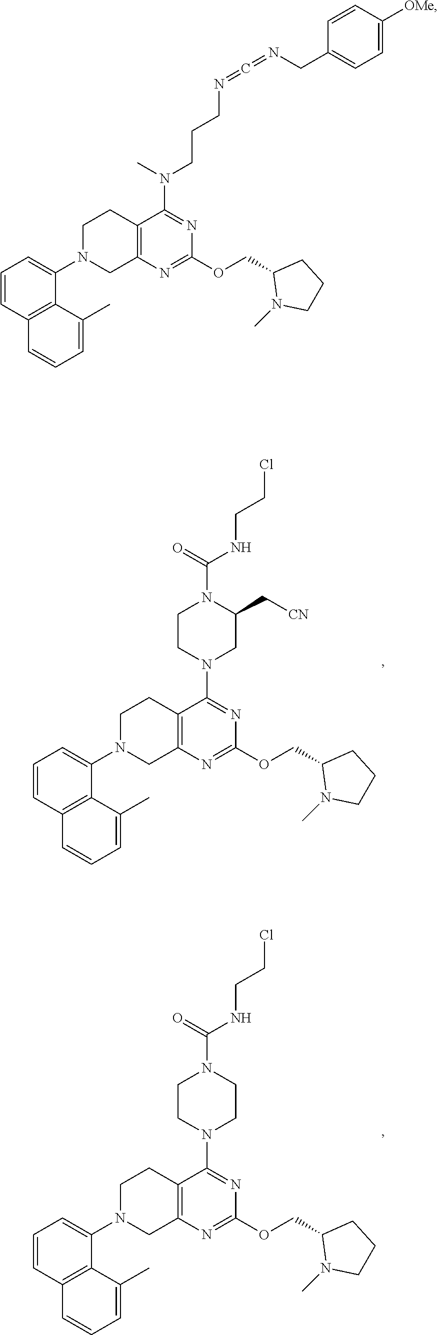 [SMILES: COc1ccc(CN=C=NCCCN(C)c2nc(OC[C@@H]3CCCN3C)nc3c2CCN(c2cccc4cccc(C)c24)C3)cc1 ; Cc1cccc2cccc(N3CCc4c(nc(OC[C@@H]5CCCN5C)nc4N4CCN(C(=O)NCCCl)[C@@H](CC#N)C4)C3)c12 ; Cc1cccc2cccc(N3CCc4c(nc(OC[C@@H]5CCCN5C)nc4N4CCN(C(=O)NCCCl)CC4)C3)c12]

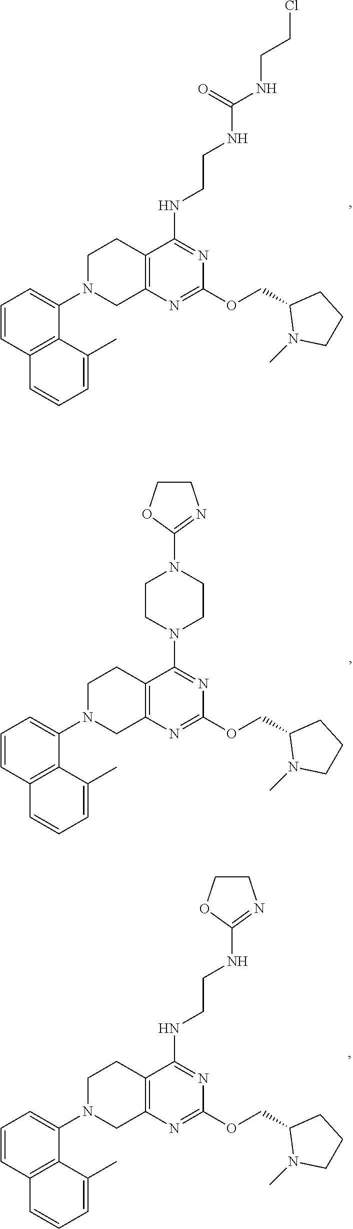 [SMILES: Cc1cccc2cccc(N3CCc4c(nc(OC[C@@H]5CCCN5C)nc4NCCNC(=O)NCCCl)C3)c12 ; Cc1cccc2cccc(N3CCc4c(nc(OC[C@@H]5CCCN5C)nc4N4CCN(C5=NCCO5)CC4)C3)c12 ; Cc1cccc2cccc(N3CCc4c(nc(OC[C@@H]5CCCN5C)nc4NCCNC4=NCCO4)C3)c12]

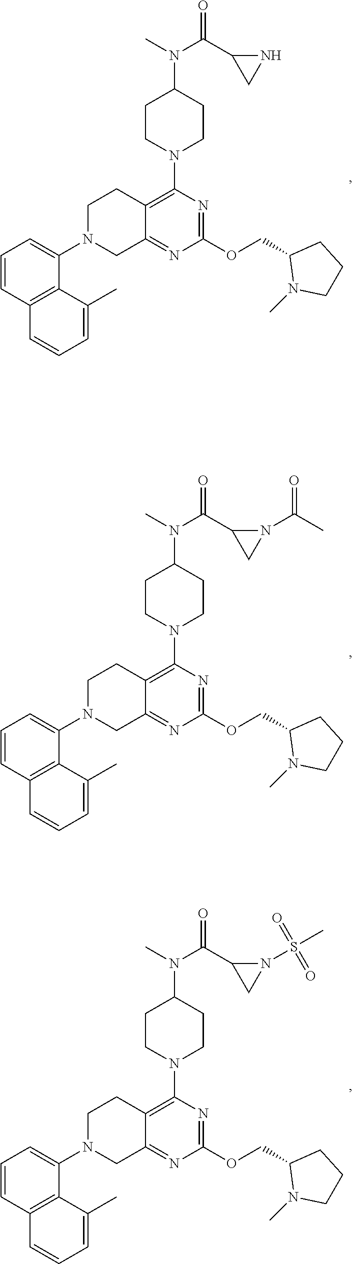 [SMILES: Cc1cccc2cccc(N3CCc4c(nc(OC[C@@H]5CCCN5C)nc4N4CCC(N(C)C(=O)C5CN5)CC4)C3)c12 ; CC(=O)N1CC1C(=O)N(C)C1CCN(c2nc(OC[C@@H]3CCCN3C)nc3c2CCN(c2cccc4cccc(C)c24)C3)CC1 ; Cc1cccc2cccc(N3CCc4c(nc(OC[C@@H]5CCCN5C)nc4N4CCC(N(C)C(=O)C5CN5S(C)(=O)=O)CC4)C3)c12]

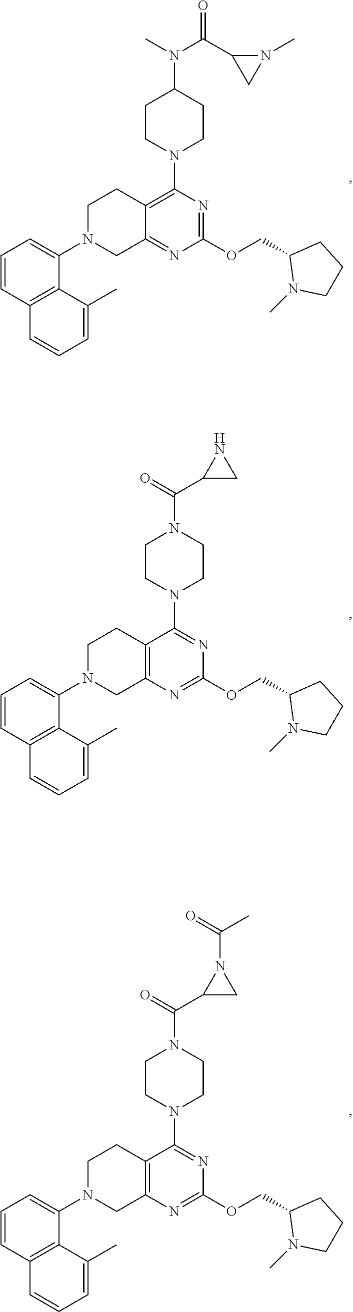 [SMILES: Cc1cccc2cccc(N3CCc4c(nc(OC[C@@H]5CCCN5C)nc4N4CCC(N(C)C(=O)C5CN5C)CC4)C3)c12 ; Cc1cccc2cccc(N3CCc4c(nc(OC[C@@H]5CCCN5C)nc4N4CCN(C(=O)C5CN5)CC4)C3)c12 ; CC(=O)N1CC1C(=O)N1CCN(c2nc(OC[C@@H]3CCCN3C)nc3c2CCN(c2cccc4cccc(C)c24)C3)CC1]

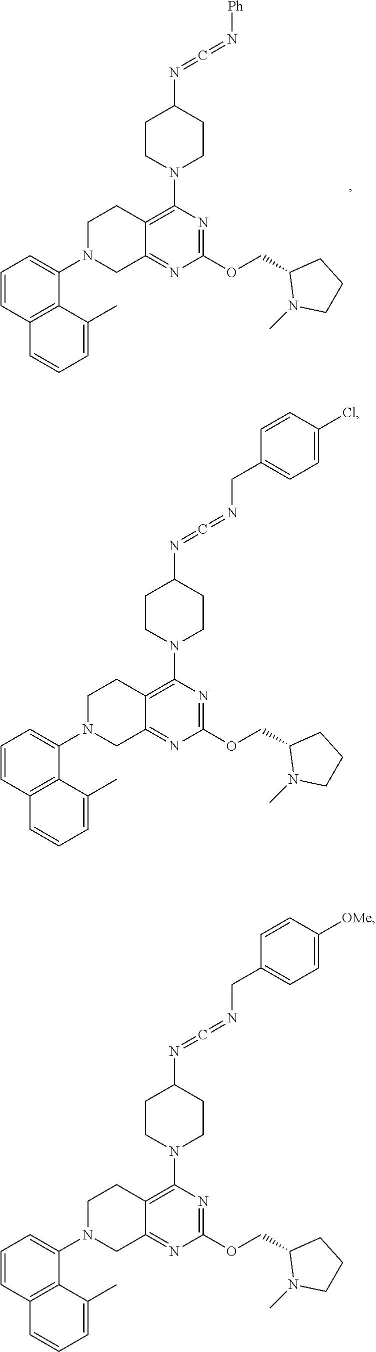 [SMILES: Cc1cccc2cccc(N3CCc4c(nc(OC[C@@H]5CCCN5C)nc4N4CCC(N=C=Nc5ccccc5)CC4)C3)c12 ; Cc1cccc2cccc(N3CCc4c(nc(OC[C@@H]5CCCN5C)nc4N4CCC(N=C=NCc5ccc(Cl)cc5)CC4)C3)c12 ; COc1ccc(CN=C=NC2CCN(c3nc(OC[C@@H]4CCCN4C)nc4c3CCN(c3cccc5cccc(C)c35)C4)CC2)cc1]

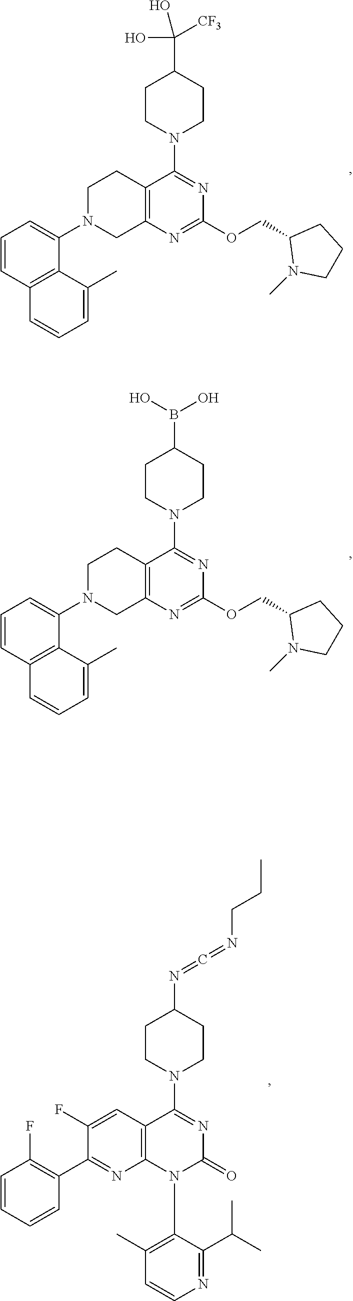 [SMILES: Cc1cccc2cccc(N3CCc4c(nc(OC[C@@H]5CCCN5C)nc4N4CCC(C(O)(O)C(F)(F)F)CC4)C3)c12 ; Cc1cccc2cccc(N3CCc4c(nc(OC[C@@H]5CCCN5C)nc4N4CCC(B(O)O)CC4)C3)c12 ; CCCN=C=NC1CCN(c2nc(=O)n(-c3c(C)ccnc3C(C)C)c3nc(-c4ccccc4F)c(F)cc23)CC1]

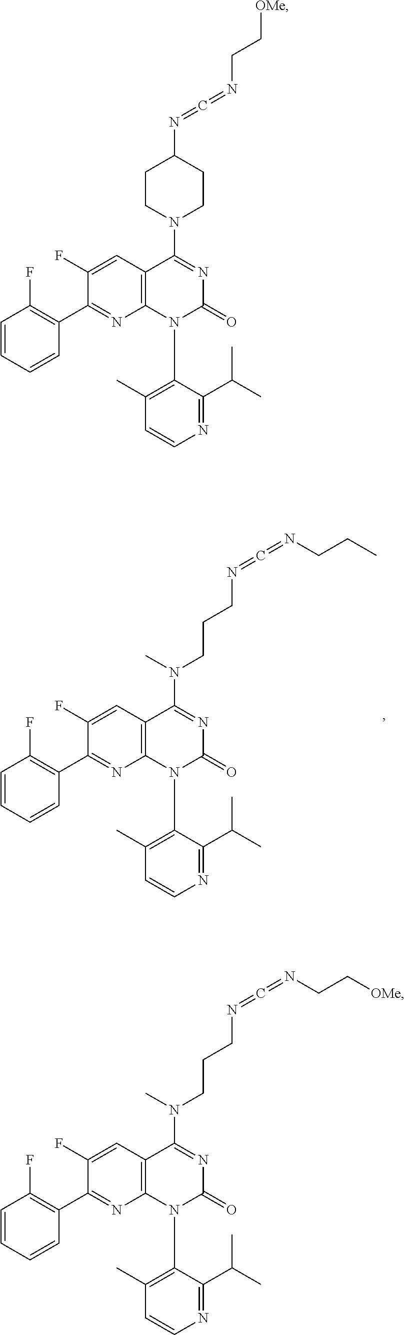 [SMILES: COCCN=C=NC1CCN(c2nc(=O)n(-c3c(C)ccnc3C(C)C)c3nc(-c4ccccc4F)c(F)cc23)CC1 ; CCCN=C=NCCCN(C)c1nc(=O)n(-c2c(C)ccnc2C(C)C)c2nc(-c3ccccc3F)c(F)cc12 ; COCCN=C=NCCCN(C)c1nc(=O)n(-c2c(C)ccnc2C(C)C)c2nc(-c3ccccc3F)c(F)cc12]

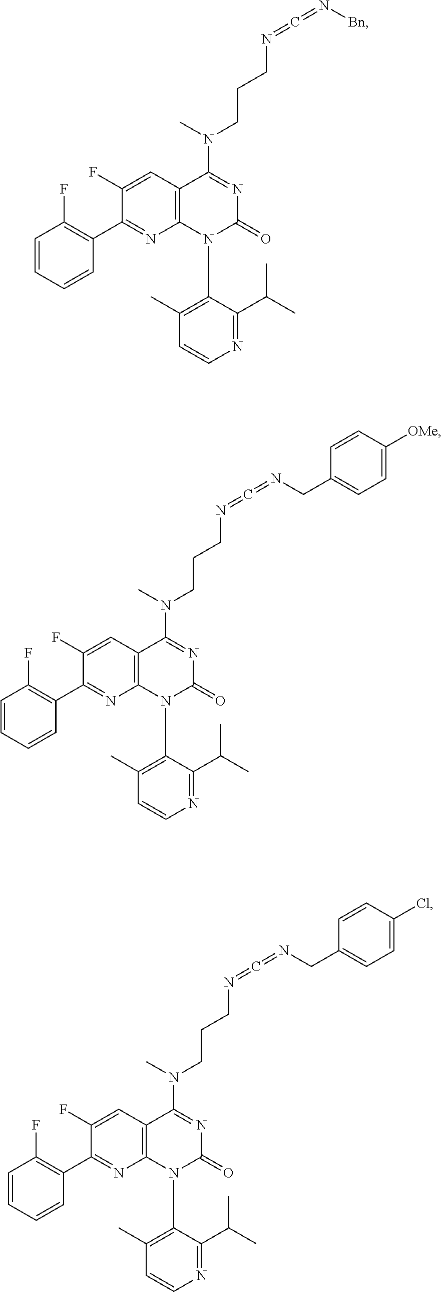 [SMILES: Cc1ccnc(C(C)C)c1-n1c(=O)nc(N(C)CCCN=C=NCc2ccccc2)c2cc(F)c(-c3ccccc3F)nc21 ; COc1ccc(CN=C=NCCCN(C)c2nc(=O)n(-c3c(C)ccnc3C(C)C)c3nc(-c4ccccc4F)c(F)cc23)cc1 ; Cc1ccnc(C(C)C)c1-n1c(=O)nc(N(C)CCCN=C=NCc2ccc(Cl)cc2)c2cc(F)c(-c3ccccc3F)nc21]

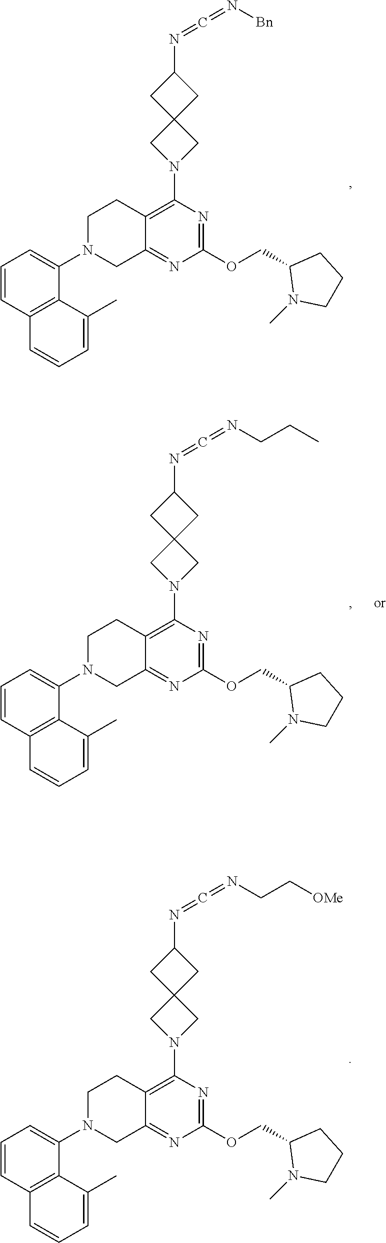 [SMILES: Cc1cccc2cccc(N3CCc4c(nc(OC[C@@H]5CCCN5C)nc4N4CC5(CC(N=C=NCc6ccccc6)C5)C4)C3)c12 ; CCCN=C=NC1CC2(C1)CN(c1nc(OC[C@@H]3CCCN3C)nc3c1CCN(c1cccc4cccc(C)c14)C3)C2 ; COCCN=C=NC1CC2(C1)CN(c1nc(OC[C@@H]3CCCN3C)nc3c1CCN(c1cccc4cccc(C)c14)C3)C2]

[75] A compound, or a pharmaceutically acceptable salt thereof, having the structure of any one of Examples 63-95 in Table 2b.

[76] A compound, or a pharmaceutically acceptable salt thereof, having the structure of any one of Examples 96-104 in Table 2c.

[77] A compound, or a pharmaceutically acceptable salt thereof, having the structure of any one of Examples 105-180 in Table 2d.

[78] A compound, or a pharmaceutically acceptable salt thereof, having the structure of any one of Examples 181-216 in Table 2e.

[79] A compound, or a pharmaceutically acceptable salt thereof, having the structure of any one of Examples 217-300 in Table 2f.

[80] A pharmaceutical composition comprising a compound, or a pharmaceutically acceptable salt thereof, of any one of paragraphs [1] to [79] and a pharmaceutically acceptable excipient.

[81] A conjugate, or salt thereof, comprising a Ras protein covalently bound to a selective cross-linking group, which selective cross-linking group is bound to a Ras binding moiety through a linker, wherein the selective cross-linking group is a carbodiimide, an aminooxazoline, a chloroethyl urea, an aziridine, a trifluoromethyl ketone, a boronic acid, a boronic ester, N-ethoxycarbonyl-2-ethoxy-1,2-dihydroquinoline (EEDQ), an iso-EEDQ or other EEDQ derivative, an epoxide, an oxazolium, or a glycal.

[82] The conjugate of paragraph [81], or a salt thereof, comprising a linker selected from the group consisting of:

(a)

-A¹-(B¹)ₐ-(C¹)_{b}-(B²)_{c}-(D)-(B³)_{d}-(C²)ₑ-(B⁴)_{f}-A²- Formula VI

wherein A¹ is a bond between the linker and the Ras binding moiety; A² is a bond between the selective cross-linking group and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; a, b, c, d, e, and f are each, independently, 0 or 1; and D is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₂₋₆ heteroarylene, optionally substituted C₃₋₈ cycloalkylene, optionally substituted C₆₋₁₂ arylene, optionally substituted C₂-C₁₀ polyethylene glycol, or optionally substituted C₁₋₁₀ heteroalkyl, or a chemical bond linking A¹-(B¹)ₐ-(C¹)_{b}-(B²)_{c}- to -(B³)_{d}-(C²)ₑ-(B⁴)_{f}-A²;

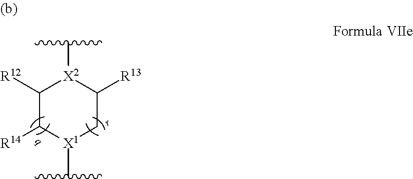

wherein q and r are, independently, 0, 1, or 2;

X¹ and X² are, independently, N or CH;

R¹² and R¹³ are, independently, hydrogen, optionally substituted C₁-C₆ alkyl or optionally substituted C₁-C₆ heteroalkyl; and

R¹⁴ is hydrogen, optionally substituted C₁-C₆ alkyl, or optionally substituted C₁-C₆ heteroalkyl, wherein R¹⁴ optionally comprises a bond to A;

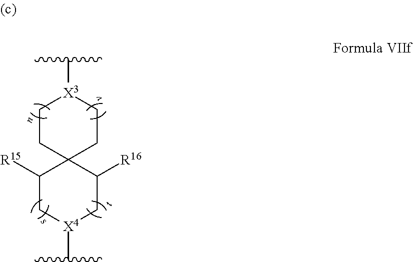

wherein s, t, u, and v are, independently, 0, 1, or 2;

X³ and X⁴ are, independently, N or CH; and

R¹⁵ and R¹⁶ are, independently, hydrogen, optionally substituted C₁-C₆ alkyl or optionally substituted C₁-C₆ heteroalkyl; and

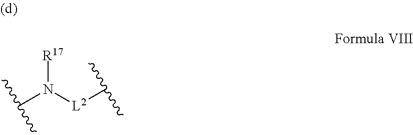

wherein R¹⁷ is hydrogen or optionally substituted C₁-C₆ alkyl; and

L² is optionally substituted C₁-C₄ alkylene.

[83] The conjugate, or salt thereof, of paragraph [81] or [82], wherein the Ras protein is K-Ras G12D, K-Ras G13D, or K-Ras G12S.

[84] The conjugate, or salt thereof, of any one of paragraphs [81] to [82], wherein the linker is bound to the Ras protein through a bond to a carboxyl group of a residue of the Ras protein.

[85] The conjugate, or salt thereof, of paragraph [83], wherein the carboxyl group of a residue of the Ras protein is the carboxyl group of an aspartic acid residue at the mutated position corresponding to position 12 or 13 of human wild-type K-Ras (SEQ ID NO: 1).

[86] A method of producing a conjugate comprising contacting a Ras protein with a compound, or a pharmaceutically acceptable salt thereof, of any one of paragraphs [1] to [79] or a pharmaceutical composition of paragraph [80] under conditions sufficient for the compound to react covalently with the Ras protein.

[87] The method of paragraph [86], wherein the Ras protein is K-Ras G12D, K-Ras G13D, or K-Ras G12S.

[88] A conjugate produced by the method of paragraph [86] or [87].

[89] A method of producing a conjugate, the method comprising contacting a Ras protein with a compound, or a pharmaceutically acceptable salt thereof, of any one of paragraphs [1] to [79] or a pharmaceutical composition of paragraph [80] under conditions suitable to permit conjugate formation.

[90] The method of paragraph [89], wherein the Ras protein is K-Ras G12D, K-Ras G13D, or K-Ras G12S.

[91] A method of treating cancer in a subject in need thereof, the method comprising administering to the subject a therapeutically effective amount of a compound, or a pharmaceutically acceptable salt thereof, of any one of paragraphs [1] to [79] or a pharmaceutical composition of paragraph [80].

[92] The method of paragraph [91], wherein the cancer is colorectal cancer, non-small cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer.

[93] The method of paragraph [91] or [92], wherein the cancer comprises a Ras mutation.

[94] The method of paragraph [93], wherein the Ras mutation is K-Ras G12D, K-Ras G13D, or K-Ras G12S.

[95] A method of treating a Ras protein-related disorder in a subject in need thereof, the method comprising administering to the subject a therapeutically effective amount of a compound, or a pharmaceutically acceptable salt thereof, of any one of paragraphs [1] to [79] or a pharmaceutical composition of paragraph [80].

[96] A method of inhibiting a Ras protein in a cell, the method comprising contacting the cell with an effective amount of a compound, or a pharmaceutically acceptable salt thereof, of any one of paragraphs 1 to [79] or a pharmaceutical composition of paragraph [80].

[97] The method of paragraph [95] or [96], wherein the Ras protein is K-Ras G12D, K-Ras G13D, or K-Ras G12S.

[98] The method of paragraph [96] or [97], wherein the cell is a cancer cell.

[99] The method of paragraph [98], wherein the cancer cell is a colorectal cancer cell, a non-small cell lung cancer cell, a pancreatic cancer cell, an appendiceal cancer cell, a melanoma cell, an acute myeloid leukemia cell, a small bowel cancer cell, an ampullary cancer cell, a germ cell cancer cell, a cervical cancer cell, a cancer cell of unknown primary origin, an endometrial cancer cell, an esophagogastric cancer cell, a GI neuroendocrine cancer cell, an ovarian cancer cell, a sex cord stromal tumor cancer cell, a hepatobiliary cancer cell, or a bladder cancer cell.

The method or use of any one of paragraphs [91] to [99], wherein the method further comprises administering an additional anticancer therapy.

The method of paragraph [100], wherein the additional anticancer therapy is an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, or a combination thereof.

The method of paragraph [100] or [101], wherein the additional anticancer therapy is a SHP2 inhibitor.

### EXAMPLES

The following examples are intended to illustrate the synthesis and use of a representative number of compounds, or a pharmaceutically acceptable salt thereof. Accordingly, the examples are intended to illustrate but not to limit the invention. Additional compounds not specifically exemplified may be synthesized using conventional methods in combination with the methods described herein.

### Abbreviations

Ac Acetyl
BnNCS Benzyl isothiocyanate
Boc tert-Butyloxycarbonyl
Cbz Benzyloxycarbonyl
CbzOSu Benzyl (2,5-dioxopyrrolidin-1-yl) carbonate
COMU (1-Cyano-2-ethoxy-2-oxoethylidenaminooxy)dimethylamino-morpholino-carbenium hexafluorophosphate
DBU 1,8-Diazabicyclo[5.4.0]undec-7-ene
DCM Dichloromethane
DMA N,N-Dimethylacetamide
DMAP N,N-Dimethylamin-4-pyridine
DMF N,N-Dimethylformamide
DMSO Dimethylsulfoxide
EDC N-(3-Dimethylaminopropy)-N′-ethyl-carbodiimide
Et Ethyl
HATU N-[(Dimethylamino)-1H-1,2,3-triazolo-[4,5-b]pyridin-1-ylmethylene]-N-methylmethanaminium hexafluorophosphate N-oxide
HOBt 1-Hydroxybenzotriazole
KHMDS Potassium bis(trimethylsilyl)amide
m-CPBA meta-Chloroperoxybenzoic acid
Me Methyl
MsCl Mesyl chloride
MTBE Methyl tert-butyl ether
NCS N-chlorosuccinimide
NMM N-methylmorpholine
n-PrNCS 1-propyl isothiocyanate
Pd₂(dba)₃ Tris(dibenzylideneacetone)dipalladium(0)
Pd(dppf)Cl₂[1,1-Bis(diphenylphosphino)ferrocene]dichloropalladium(II)
Ph₂NTf N-Phenyl-bis(trifluoromethanesulfonimide)
Pr Propyl
RuPhos Dicyclohexyl(2′,6′-diisopropoxy-[1,1′-biphenyl]-2-yl)phosphane
T₃P Propanephosphonic acid anhydride
TBAF Tetrabutylammonium fluoride
TBDPSCl tert-Butyl(chloro)diphenylsilane
Tf Triflate
TFA Trifluoroacetic acid
THF Tetrahydrofuran
Trt Trityl
TsOH Toluenesulfonic acid

### Intermediate 1—Synthesis of (2S,3S)-1-((S)-tert-butylsulfinyl)-3-phenylaziridine-2-carboxylic Acid

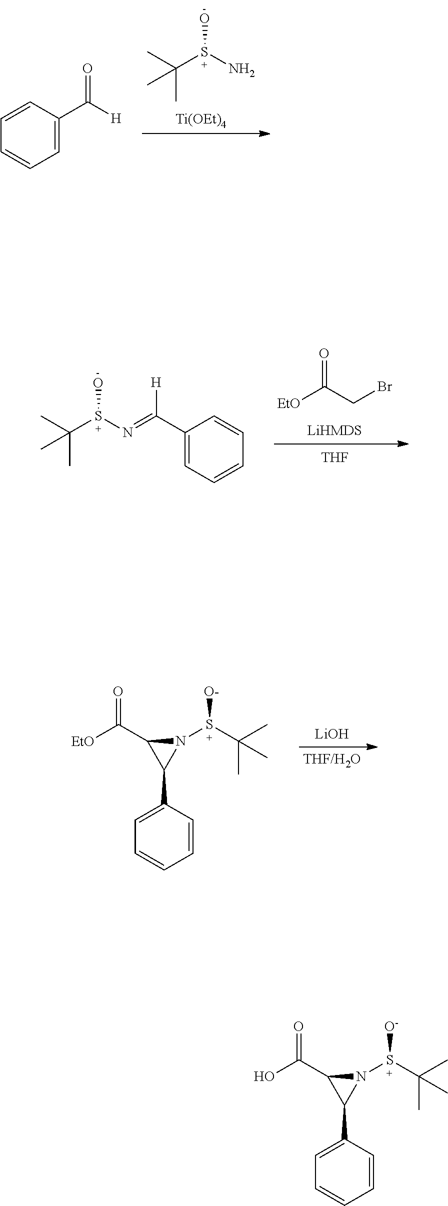

### Step 1: Synthesis of (S,E)-N-benzylidene-2-methylpropane-2-sulfinamide

A solution of (S)-2-methylpropane-2-sulfinamide (2.50 g, 20.6 mmol), titanium ethoxide (9.41 g, 41.25 mmol) and benzaldehyde (2.19 g, 20.7 mmol) was heated at 70° C. for 1 h, cooled, and diluted with H₂O (250 mL). The aqueous layer was extracted with EtOAc (3×80 mL) and the combined organic layers were washed with brine (2×100 mL), dried with Na₂SO₄, filtered and concentrated under reduced pressure to afford the desired product (4.3 g, crude) which was used without further purification. LCMS (ESI) m/z: [M+H] calcd for C₁₁H₁₅NOS: 210.10; found 210.2.

### Step 2: Synthesis of ethyl (2S,3S)-1-((S)-tert-butylsulfinyl)-3-phenylaziridine-2-carboxylate

To a solution of ethyl bromoacetate (798 mg, 4.78 mmol) in THF (15 mL) at −78° C. was added LiHMDS (1M in THF, 4.78 mL, 4.78 mmol). After 1 h, (S,E)-N-benzylidene-2-methylpropane-2-sulfinamide (500 mg, 2.39 mmol) in THF (5 mL) was added in portions over 20 min. The reaction mixture was stirred at −78° C. for 2 h and then quenched by the addition of sat. NH₄Cl. The aqueous layer was extracted with EtOAc (3×40 mL) and the combined organic layers were washed with brine (2×30 mL), dried with Na₂SO₄, filtered, and concentrated under reduced pressure. Purification by reverse phase chromatography (30→40% MeCN/H₂O, 0.1% HCO₂H) afforded the desired product (480 mg, 61% yield).

LCMS (ESI) m/z: [M+H] calcd for C₁₅H₂₁NO₃S: 296.13; found 296.2.

### Step 3: Synthesis (2S,3S)-1-((S)-tert-butylsulfinyl)-3-phenylaziridine-2-carboxylic Acid

To a solution of ethyl (2S,3S)-1-((S)-tert-butylsulfinyl)-3-phenylaziridine-2-carboxylate (600 mg, 2.03 mmol) in THF (4.0 mL) at 0° C. was added a solution of OH (97.2 mg, 4.06 mmol) in H₂O (4.0 mL). The resulting mixture was stirred for 2 h at 0° C. and then acidified to pH 5 with 1 M HCl. The aqueous layer was extracted with EtOAc (3×40 mL) and the combined organic layers were washed with brine (2×20 mL), dried with Na₂SO₄, filtered, and concentrated under reduced pressure to afford the desired compound (450 mg, crude) which was used without further purification. LCMS (ESI) m/z: [M+H] calcd for C₁₃H₁₇NO₃S: 268.10; found 268.1.

### Intermediate 2—Synthesis of (2R,3R)-1-((R)-tert-butylsulfinyl)-3-phenylaziridine-2-carboxylic Acid

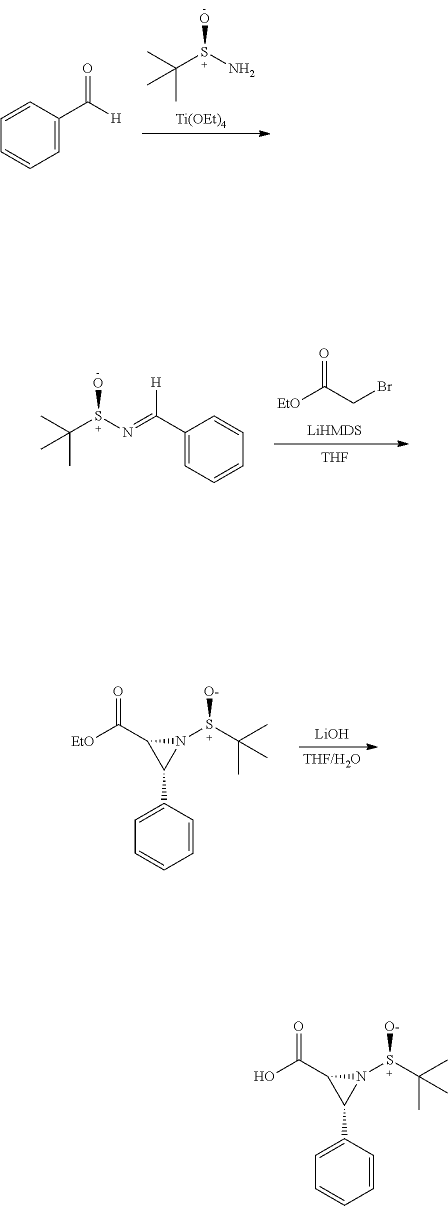

### Step 1: Synthesis (R,E)-N-benzylidene-2-methylpropane-2-sulfinamide

A solution (R)-2-methylpropane-2-sulfinamide (2.50 g, 20.6 mmol), titanium tetraethoxide (9.41 g, 41.3 mmol) and benzaldehyde (2.19 g, 20.6 mmol) was heated 70° C. for 1 h, cooled, and diluted with H₂O (250 mL). The aqueous layer was extracted with EtOAc (3×90 mL) and the combined organic layers were washed with brine (2×100 mL), dried with Na₂SO₄, filtered and concentrated under reduced pressure to afford the desired product (4.2 g, crude) which was used without further purification. LCMS (ESI) m/z: [M+H] calcd for C₁₁H₁₅NOS: 210.10; found 210.1.

### Step 2: Synthesis of ethyl (2R,3R)-1-((R)-tert-butylsulfinyl)-3-phenylaziridine-2-carboxylate

To a solution of ethyl bromoacetate (6.38 g, 38.2 mmol) in THF (150 mL) at −78° C. was added LiHMDS (1M in THF, 7.19 mL, 42.9 mmol). After 1 h, (R,E)-N-benzylidene-2-methylpropane-2-sulfinamide (4.0 g, 19.1 mmol) in THF (50 mL) was added in portions over 20 min. The reaction mixture was stirred at −78° C. for 2 h and then quenched by the addition of sat. NH₄Cl. The aqueous layer was extracted with EtOAc (3×80 mL) and the combined organic layers were washed with brine (2×60 mL), dried with Na₂SO₄, filtered and concentrated under reduced pressure. Purification by reverse phase chromatography (30→40% MeCN/H₂O, 0.1% HCO₂H) afforded the desired product (3.9 g, 62% yield). LCMS (ESI) m/z: [M+H] calcd for C₁₅H₂₁NO₃S: 296.13; found 296.2.

### Step 3: Synthesis (2R,3R)-1-((R)-tert-butylsulfinyl)-3-phenylaziridine-2-carboxylic Acid

To a solution of ethyl (2R,3R)-1-((R)-tert-butylsulfinyl)-3-phenylaziridine-2-carboxylate (200 mg, 0.677 mmol) in THF (1.5 mL) at 0° C. was added a solution of OH (32.4 mg, 1.35 mmol) in H₂O (1.3 mL). The resulting mixture was stirred for 2 h at 0° C. and then acidified to pH 5 with 1M HCl. The aqueous layer was extracted with EtOAc (3×20 mL) and the combined organic layers were washed with brine (2×10 mL), dried with Na₂SO₄, filtered, and concentrated under reduced pressure to afford the desired compound (220 mg, crude) which was used without further purification. LCMS (ESI) m/z: [M+H] calcd for C₁₃H₁₇NO₃S: 268.10; found 268.4.

### Intermediate 3—Synthesis of (2R,3S)-3-phenylaziridine-2-carboxylic Acid

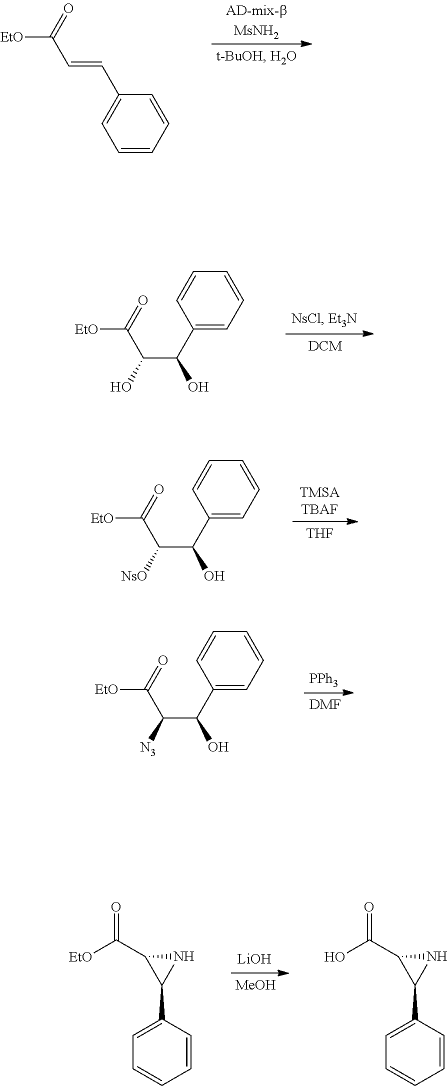

### Step 1: Synthesis of ethyl (2S,3R)-2,3-dihydroxy-3-phenylpropanoate

To a solution of ethyl cinnamate (2.0 g, 11.4 mmol) in t-BuOH (35.0 mL) and H₂O (35.0 mL) at 0° C. was added AD-mix-β (15.83 g, 20.32 mmol), and methanesulfonamide (1.08 g, 11.3 mmol). The reaction mixture was stirred at room temperature for 16 h. The reaction was cooled to 0° C. and quenched with aq. KHSO₄. The resulting mixture was extracted with EtOAc (3×100 mL) and the combined organic layers were washed with brine (2×90 mL), dried over Na₂SO₄, filtered, and concentrated under reduced pressure. The residue was purified by normal phase chromatography (50% EtOAc/pet. ether) to afford the desired product (2.2 g, 82% yield) as a solid.

### Step 2: Synthesis of ethyl (2S,3R)-3-hydroxy-2-(((4-nitrophenyl)sulfonyloxy)-3-phenylpropanoate

To a solution of ethyl (2S,3R)-2,3-dihydroxy-3-phenylpropanoate (2.0 g, 9.5 mmol) and Et₃N (3.97 mL, 28.5 mmol) in DCM (30.0 mL) at 0° C. was added 4-nitrobenzenesulfonyl chloride (2.11 g, 9.51 mmol). The resulting mixture was stirred for 1 h and was then diluted with H₂O (300 mL). The mixture was extracted with DCM (3×100 mL) and the combined organic layers were washed with brine (2×100 mL), dried over Na₂SO₄, filtered, and concentrated under reduced pressure. The residue was purified by prep-TLC (50% EtOAc/pet. ether) to afford the desired product (2.8 g, 67% yield) as a solid.

### Step 3: Synthesis of ethyl (2R,3R)-2-azido-3-hydroxy-3-phenylpropanoate

To a solution of ethyl (2S,3R)-3-hydroxy-2-(((4-nitrophenyl)sulfonyl)oxy)-3-phenylpropanoate (2.80 g, 7.08 mmol) in THF (30 mL) at room temperature was added trimethylsilyl azide (1.63 g, 14.2 mmol) and TBAF (1M in THF, 14.16 mL, 14.16 mmol). The reaction mixture was heated to 60° C. and was stirred for 16 h. The reaction mixture was then cooled to room temperature, diluted with H₂O (150 mL), and extracted with EtOAc (3×50 mL). The combined organic layers were washed with brine (2×30 mL), dried over Na₂SO₄, filtered, and concentrated under reduced pressure. The residue was purified by normal phase chromatography (50% EtOAc/pet. ether) to afford the desired product (1.2 g, 64% yield) as an oil.

### Step 4: Synthesis of ethyl (2R,3S)-3-phenylaziridine-2-carboxylate

To a solution of ethyl (2R,3R)-2-azido-3-hydroxy-3-phenylpropanoate (1.20 g, 5.10 mmol) in DMF (15.0 mL) was added PPh₃ (1.61 g, 6.12 mmol). The reaction mixture was stirred at room temperature for 30 min and then heated to 80° C. for an additional 16 h. The reaction mixture was then cooled to room temperature, diluted with H₂O (100 mL), and extracted with EtOAc (3×40 mL). The combined organic layers were washed with brine (20 mL), dried over Na₂SO, filtered, and concentrated under reduced pressure. The residue was purified by normal phase chromatography (16% EtOAc/pet. ether) to afford the desired product (620 mg, 57% yield) as an oil. LCMS (ESI) m/z: [M+H] calcd for C₁₁H₁₃NO₂: 192.10; found 192.0.

### Step 5: Synthesis of (2R,3S)-3-phenylaziridine-2-carboxylic Acid

To a solution of ethyl (2R,3S)-3-phenylaziridine-2-carboxylate (0.100 g, 0.523 mmol) in MeOH (0.70 mL) at 0° C. was added a solution of LiOH (18.8 mg, 0.784 mmol) in H₂O (0.70 mL). The reaction mixture was stirred for 1 h. The mixture was then diluted with MeCN (10 mL), and the resulting precipitate was collected by filtration and washed with MeCN (2×10 mL) to afford the crude desired product (70 mg) as a solid. LCMS (ESI) m/z: [M+H] calcd for C₉H₉NO₂: 164.07; found 164.0.

### Intermediate 4—Synthesis of (2S,3R)-3-phenylaziridine-2-carboxylic Acid

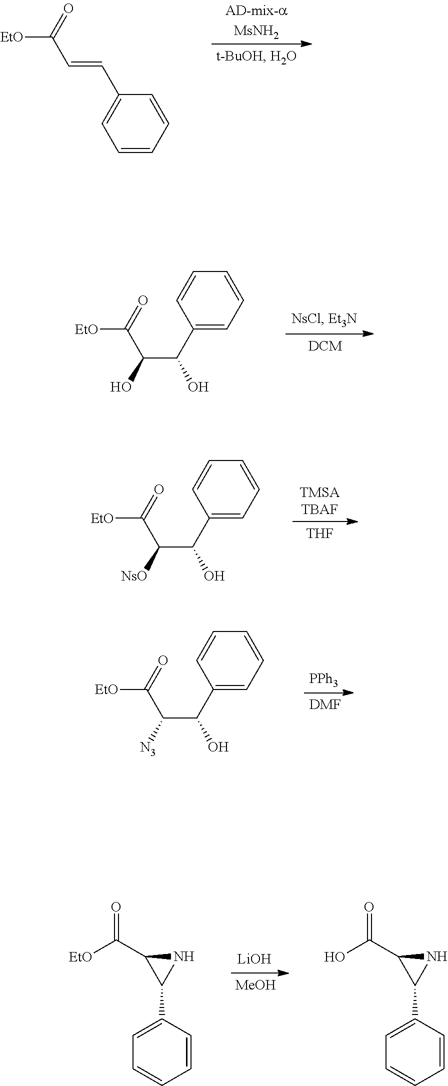

### Step 1: Synthesis of ethyl (2R,3S)-2,3-dihydroxy-3-phenylpropanoate

To a solution of ethyl cinnamate (2.0 g, 11.4 mmol) in t-BuOH (35.0 mL) and H₂O (35.0 mL) at 0° C. was added AD-mix-α (15.83 g, 20.32 mmol), and methanesulfonamide (1.08 g, 11.3 mmol). The reaction mixture was stirred at room temperature for 16 h. The reaction was cooled to 0° C. and quenched with aq. KHSO₄. The resulting mixture was extracted with EtOAc (3×100 mL) and the combined organic layers were washed with brine (2×80 mL), dried over Na₂SO₄, filtered, and concentrated under reduced pressure. The residue was purified by normal phase chromatography (50% EtOAc/pet. ether) to afford the desired product (2.2 g, 82% yield) as a solid.

### Step 2: Synthesis of ethyl (2R,3S)-3-hydroxy-2-(((4-nitrophenyl)sulfonyl)oxy)-3-phenylpropanoate

To a solution of ethyl (2R,3S)-2,3-dihydroxy-3-phenylpropanoate (2.10 g, 9.99 mmol) and Et₃N (4.18 mL, 29.9 mmol) in DCM (30.0 mL) at 0° C. was added 4-nitrobenzenesulfonyl chloride (2.21 g, 9.99 mmol). The resulting mixture was stirred for 1 h and was then diluted with H₂O (200 mL). The mixture was extracted with DCM (3×80 mL) and the combined organic layers were washed with brine (2×80 mL), dried over Na₂SO₄, filtered, and concentrated under reduced pressure. The residue was purified by prep-TLC (50% EtOAc/pet. ether) to afford the desired product (3.0 g, 68% yield) as a solid.

### Step 3: Synthesis of ethyl (2S,3S)-2-azido-3-hydroxy-3-phenylpropanoate

To a solution of ethyl (2R,3S)-3-hydroxy-2-(((4-nitrophenyl)sulfonyl)oxy)-3-phenylpropanoate (3.0 g, 7.59 mmol) in THF (30 mL) at room temperature was added trimethylsilyl azide (1.75 g, 15.2 mmol) and TBAF (1M in THF, 15.18 mL, 15.18 mmol). The reaction mixture was heated to 60° C. and was stirred for 16 h. The reaction mixture was then cooled to room temperature, diluted with H₂O (150 mL), and extracted with EtOAc (3×50 mL). The combined organic layers were washed with brine (2×30 mL), dried over Na₂SO₄, filtered, and concentrated under reduced pressure. The residue was purified by normal phase chromatography (50% EtOAc/pet. ether) to afford the desired product (1.4 g, 70% yield) as an oil.

### Step 4: Synthesis of ethyl (2S,3R)-3-phenylaziridine-2-carboxylate

To a solution of ethyl (2S,3S)-2-azido-3-hydroxy-3-phenylpropanoate (1.40 g, 5.95 mmol) in DMF (20.0 mL) was added PPh₃ (1.87 g, 7.14 mmol). The reaction mixture was stirred at room temperature for 30 min and then heated to 80° C. for an additional 16 h. The reaction mixture was then cooled to room temperature, diluted with H₂O (150 mL), and extracted with EtOAc (3×50 mL). The combined organic layers were washed with brine (40 mL), dried over Na₂SO, filtered, and concentrated under reduced pressure. The residue was purified by normal phase chromatography (16% EtOAc/pet. ether) to afford the desired product (720 mg, 56% yield) as an oil. LCMS (ESI) m/z: [M+H] calcd for C₁₁H₁₃NO₂: 192.10; found 192.0.

### Step 5: Synthesis of (2S,3R)-3-phenylaziridine-2-carboxylic Acid

To a solution of ethyl (2S,3R)-3-phenylaziridine-2-carboxylate (0.100 g, 0.523 mmol) in MeOH (0.70 mL) at 0° C. was added a solution of LiOH (18.8 mg, 0.784 mmol) in H₂O (0.70 mL). The reaction mixture was stirred for 1 h. The mixture was then diluted with MeCN (10 mL), and the resulting precipitate was collected by filtration and washed with MeCN (2×10 mL) to afford the crude desired product (68 mg) as a solid. LCMS (ESI) m/z: [M+H] calcd for C₉H₉NO₂: 164.07; found 164.0.

### Intermediate 5—Synthesis of (2R,3R)-1-((R)-tert-butylsulfinyl)-3-methylaziridine-2-carboxylic Acid

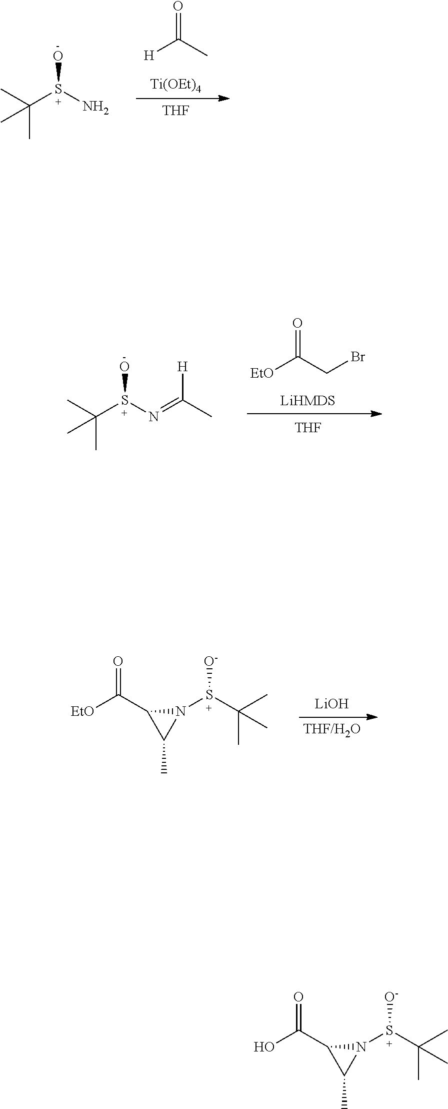

### Step 1: Synthesis of (R,E)-N-ethylidene-2-methylpropane-2-sulfinamide

To a solution of (R)-2-methylpropane-2-sulfinamide (3.0 g, 24.75 mmol) and tetraethoxytitanium (1.7 g, 7.43 mmol) in THF (30 mL) at 0° C. was added acetaldehyde (218.1 mg, 4.95 mmol). The resulting mixture was stirred for 20 min and was then quenched with H₂O (100 mL). The suspension was filtered, and the filter cake washed with EtOAc (3×100 mL). The aqueous layer was extracted with EtOAc (3×100 mL) and the combined organic layers were washed with brine (3×100 mL), dried over Na₂SO₄, filtered, and concentrated under reduced pressure. Purification by normal phase chromatography (9% EtOAc/pet. ether) afforded desired product (3 g, 82% yield). LCMS (ESI) m/z: [M+H] calcd for C₆H₁₃NOS: 148.08; found 148.0.

### Step 2: Synthesis of ethyl (2R,3R)-1-((R)-tert-butylsulfinyl)-3-methylaziridine-2-carboxylate

To a solution of 1M LiHMDS (40.75 mL, 40.75 mmol) in THF (30.0 mL) at −78° C. was added ethyl bromoacetate (6.80 g, 40.75 mmol). The resulting mixture was stirred for 1 h. To the reaction mixture was then added (R,E)-N-ethylidene-2-methylpropane-2-sulfinamide (3.0 g, 20.38 mmol). The resulting mixture was stirred for 2 h at −78° C. and then quenched with H₂O (300 mL). The aqueous layer was extracted with EtOAc (3×300 mL) and the combined organic layers were washed with brine (3×100 mL), dried over Na₂SO₄, filtered, and concentrated under reduced pressure. The residue was purified by reverse phase chromatography (10→50% MeCN/H₂O) to afford the desired product (1.4 g, 29.5% yield). LCMS (ESI) m/z: [M+H] calcd for C₁₀H₁₉NO₃S: 234.12; found 234.1.

### Step 3: Synthesis of (2R,3R)-1-((R)-tert-butylsulfinyl)-3-methylaziridine-2-carboxylic Acid

To a solution of ethyl (2R,3R)-1-((R)-tert-butylsulfinyl)-3-methylaziridine-2-carboxylate (1.0 g, 4.29 mmol) in THF (6.4 mL) and H₂O (6.4 mL) at 0° C. was added LiOH.H₂O (539.5 mg, 12.86 mmol). The resulting mixture was warmed to room temperature and stirred for 2 h and was then neutralized to pH 5 with HCl (aq.) and sat. NH₄Cl (aq.). The aqueous layer was extracted with EtOAc (3×10 mL) and the combined organic layers were washed with brine, dried over Na₂SO₄, filtered, and concentrated under reduced pressure to afford the desired crude product (489 mg, 55.6% yield). LCMS (ESI) m/z: [M+H] calcd for C₈H₁₅NO₃S: 206.09; found 206.0.

### Intermediate 6—Synthesis of (2S,3S)-1-(S)-tert-butylsulfinyl)-3-methylaziridine-2-carboxylic Acid

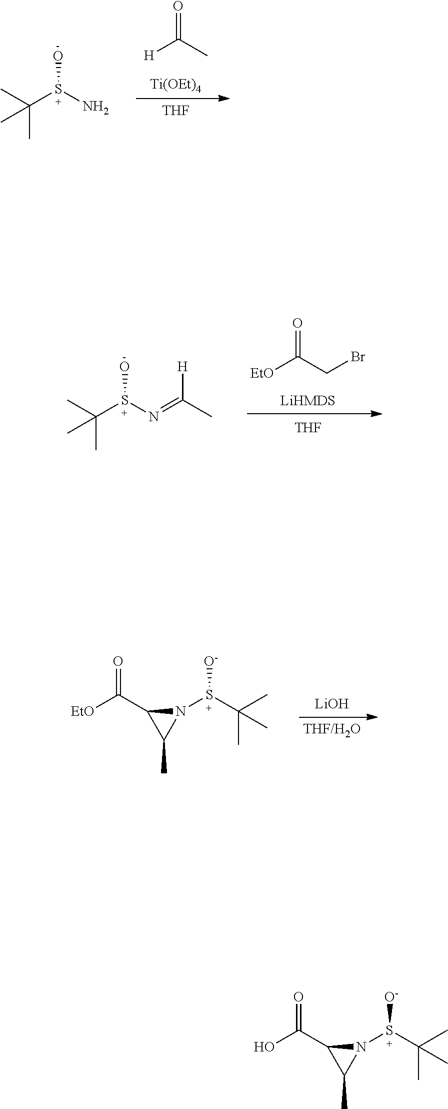

### Step 1: Synthesis of (S,E)-N-ethylidene-2-methylpropane-2-sulfinamide

To a mixture of (S)-2-methylpropane-2-sulfinamide (5.0 g, 41.25 mmol) and tetraethoxytitanium (18.82 g, 82.51 mmol) at 0° C. was added acetaldehyde (3.63 g, 82.51 mmol). The resulting mixture was warmed to room temperature and stirred for 30 min and was then quenched with H₂O (100 mL). The suspension was filtered, and the filter cake washed with EtOAc (3×100 mL). The aqueous layer was extracted with EtOAc (3×100 mL) and the combined organic layers were washed with brine (3×100 mL), dried over Na₂SO₄, filtered, and concentrated under reduced pressure to afford desired crude product (3.9 g, 64% yield). LCMS (ESI) m/z: [M+H] calcd for C₆H₁₃NOS: 148.08; found 148.2.

### Step 2: Synthesis of ethyl (2S,3S)-1-((S)-tert-butylsulfinyl)-3-methylaziridine-2-carboxylate

To a solution of 1M LiHMDS (40.75 mL, 40.75 mmol) in THF (30.0 mL) at −78° C. was added ethyl bromoacetate (6.80 g, 40.75 mmol). The resulting mixture was stirred for 1 h. To the reaction mixture was then added (S,E)-N-ethylidene-2-methylpropane-2-sulfinamide (3.0 g, 20.38 mmol). The resulting mixture was stirred for 2 h at −78° C. and then quenched with H₂O. The aqueous layer was extracted with EtOAc (3×200 mL) and the combined organic layers were washed with brine (3×300 mL), dried over Na₂SO₄, filtered, and concentrated under reduced pressure. The residue was purified by reverse phase chromatography (10→50% MeCN/H₂O) to afford the desired product (2 g, 42% yield). LCMS (ESI) M/&: [M+H] calcd for C₁₀H₁₉NO₃S: 234.12; found 234.0.

### Step 3: Synthesis of (2S,3S)-1-((S)-tert-butylsulfinyl)-3-methylaziridine-2-carboxylic Acid

To a solution of ethyl (2S,3S)-1-((S)-tert-butylsulfinyl)-3-methylaziridine-2-carboxylate (80.0 mg, 0.34 mmol) in THF (1.0 mL) and H₂O (0.2 mL) at 0° C. was added LiOH.H₂O (32.9 mg, 1.37 mmol). The resulting mixture was warmed to room temperature and stirred for 4 h and was then acidified to pH 3 with HCl (aq.). The aqueous layer was extracted with EtOAc (3×10 mL) and the combined organic layers were washed with brine, dried over Na₂SO₄, filtered, and concentrated under reduced pressure to afford the desired crude product (70 mg, 99% yield). LCMS (ESI) m/z: [M+H] calcd for C₈H₁₅NO₃S: 206.09; found 206.0.

### Intermediate 7—Synthesis of (2R,3R)-3-isopropyl-1-tritylaziridine-2-carboxylic Acid

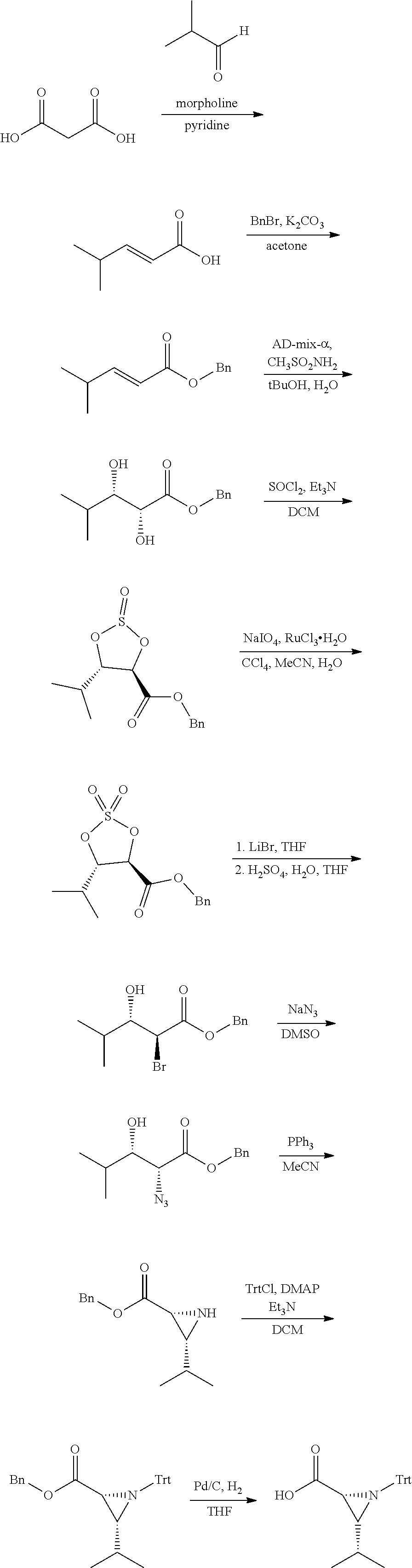

### Step 1: Synthesis of (E)-4-methylpent-2-enoic Acid

Two batches of a solution of malonic acid (25.0 ml, 240 mmol), isobutyraldehyde (34.7 mL, 380 mmol) and morpholine (380 μL, 4.32 mmol) in pyridine (75 mL) were stirred for 24 h then were heated to 115° C. and stirred for 12 h. The combined reaction mixtures were poured into H₂SO₄ (1M, 800 mL) and extracted into EtOAc (3×300 mL). The combined organic layers were washed with brine (300 mL), dried over Na₂SO₄, filtered, and concentrated under reduced pressure. The residue was dissolved in NaOH (1 M, 500 mL), washed with EtOAc (2×200 mL), acidified to pH=4-2 with HCl (4M), and extracted into EtOAc (3×300 mL). The combined organic layers were washed with brine (300 mL), dried over Na₂SO₄, filtered, and concentrated under reduced pressure which afforded product (54 g, 98% yield).

### Step 2: Synthesis of benzyl (E)-4-methylpent-2-enoate

To two batches of a solution of (E)-4-methylpent-2-enoic acid (6.25 mL, 52.6 mmol) in acetone (90 mL) was added K₂CO₃ (13.8 g, 100 mmol) and the mixtures were stirred for 30 min. Then a solution of benzyl bromide (6.31 mL, 53.1 mmol) in acetone (10 mL) was added and the mixtures were heated to 75° C. for 5 h. The reaction mixtures were cooled to room temperature and concentrated under reduced pressure. The residue was dissolved in EtOAc (200 mL) and H₂O (200 mL) then extracted into EtOAc (2×200 mL). The combined organic layers were washed with brine (300 mL), dried over Na₂SO₄, filtered, and concentrated under reduced pressure. Purification by silica gel chromatography (0→10% EtOAc/pet. ether) afforded product (9.0 g, 42% yield).

### Step 3: Synthesis of benzyl (2R,3S)-2,3-dihydroxy-4-methylpentanoate

To a solution of AD-mix-α (61.7 g) and methanesulfonamide (4.19 g, 44.1 mmol) in tert-BuOH (225 mL) and H₂O (225 mL) was added benzyl (E)-4-methylpent-2-enoate (9 g, 44.1 mmol). The mixture was stirred at room temperature for 12 h then Na₂SO₃ (67.5 g) was added and stirred for 30 min. The reaction mixture was diluted with EtOAc (300 mL) and H₂O (300 mL) and extracted into EtOAc (3×300 mL), washed with brine (300 mL), dried over Na₂SO₄, filtered, and concentrated under reduced pressure. Purification by silica gel chromatography (0→25% EtOAc/pet. ether) afforded product (8.3 g, 79% yield). LCMS (ESI) m/z: [M+Na] calcd for C₁₃H₁₈O₄: 261.11; found 261.0.

### Step 4: Synthesis of benzyl (4R,5S)-5-isopropyl-1,3,2-dioxathiolane-4-carboxylate 2-oxide

To a solution of benzyl (2R,3S)-2,3-dihydroxy-4-methylpentanoate (10 g, 42.0 mmol) in DCM (100 mL) at 0° C. was added Et₃N (17.5 mL, 126 mmol) and SOCl₂ (4.26 ml, 58.8 mmol). The reaction mixture was stirred 30 min then was diluted with DCM (30 mL) and H₂O (100 mL), extracted into DCM (3×50 mL), washed with brine (100 mL), dried over Na₂SO₄, filtered, and concentrated under reduced pressure which afforded product (11.0 g, 92% yield).

### Step 5: Synthesis of benzyl (4R,5S)-5-isopropyl-1,3,2-dioxathiolane-4-carboxylate 2,2-dioxide

To a solution of benzyl (4R,5S)-5-isopropyl-1,3,2-dioxathiolane-4-carboxylate 2-oxide (11 g, 38.7 mmol) in H₂O (250 mL), MeCN (125 mL), and CCl₄ (125 mL) was added NaIO₄ (3.22 mL, 58.0 mmol) and RuCl₃.H₂O (872 mg, 3.87 mmol). The mixture was stirred at room temperature for 1 h then was diluted with EtOAc (200 mL) and H₂O (50 mL), filtered, and the filtrate was extracted into EtOAc (3×200 mL). The combined organic layers were washed sequentially with brine (200 mL) and sat. aq. Na₂CO₃ (300 mL), dried over Na₂SO₄, filtered, and concentrated under reduced pressure. Purification by silica gel chromatography (0→17% EtOAc/pet. ether) afforded product (11 g, 95% yield).

### Step 6: Synthesis of benzyl (2S,3S)-2-bromo-3-hydroxy-4-methylpentanoate

To a solution of benzyl (4R,5S)-5-isopropyl-1,3,2-dioxathiolane-4-carboxylate 2,2-dioxide (11 g, 36.6 mmol) in THF (520 mL) was added LiBr (3.49 mL, 139 mmol). The reaction mixture was stirred at room temperature for 5 h and then concentrated under reduced pressure. The residue was diluted in THF (130 mL) and H₂O (65 mL), cooled to 0° C., then H₂SO₄ solution (20% aq., 1.3 L) was added and the mixture was warmed to room temperature and stirred for 24 h. The mixture was diluted with EtOAc (1.0 L), extracted into EtOAc (2×300 mL), washed sequentially with Na₂CO₃ (sat. aq., 300 mL) and brine (300 mL), then was concentrated under reduced pressure. Purification by silica gel chromatography (0→17% EtOAc/pet. ether) afforded product (10 g, 81% yield).

### Step 7: Synthesis of benzyl (2R,3S)-2-azido-3-hydroxy-4-methylpentanoate

To a solution of benzyl (2S,3S)-2-bromo-3-hydroxy-4-methylpentanoate (10 g, 33.2 mmol) in DMSO (100 mL) was added NaN₃ (4.32 g, 66.4 mmol). The reaction mixture was stirred at room temperature for 12 h then was diluted with EtOAc (300 mL) and H₂O (200 mL). The aqueous phase was extracted into EtOAc (2×200 mL), washed with brine (200 mL), dried over Na₂SO₄, filtered, and concentrated under reduced pressure. Purification by silica gel chromatography (0→17% EtOAc/pet. ether) afforded product (7.5 g, 79% yield).

### Step 8 Synthesis of benzyl (2R,3R)-3-isopropylaziridine-2-carboxylate

To a solution of benzyl (2R,3S)-2-azido-3-hydroxy-4-methylpentanoate (7.5 g, 28.5 mmol) in MeCN (150 mL) was added PPh₃ (7.70 g, 29.3 mmol). The reaction mixture was stirred at room temperature for 1 h and then heated to 70° C. and stirred for 4 h. The reaction mixture was concentrated under reduced pressure and purification by silica gel chromatography (0→17% EtOAc/pet. ether) afforded product (4.5 g, 66% yield). LCMS (ESI) m/z: [M+H] calcd for C₁₃H₁₇NO₂: 220.13; found 220.0.

### Step 9: Synthesis of benzyl (2R,3R)-3-isopropyl-1-tritylaziridine-2-carboxylate

To a solution of benzyl (2R,3R)-3-isopropylaziridine-2-carboxylate (2 g, 9.12 mmol) in DCM (30 mL) at 0° C. was added Et₃N (3.81 mL, 27.4 mmol) and trityl chloride (3.05 g, 10.9 mmol) followed by DMAP (111 mg, 912 μmol). The reaction mixture was stirred at 0° C. for 1 h and then was diluted with DCM (50 mL) and H₂O (50 mL) then extracted into DCM (2×30 mL). The combined organic layers were washed with brine (50 mL), dried over Na₂SO₄, filtered, and concentrated under reduced pressure. Purification by silica gel chromatography (0→25% DCM/pet. ether) afforded product (3.1 g, 72% yield).

### Step 10: Synthesis of (2R,3R)-3-isopropyl-1-tritylaziridine-2-carboxylic Acid

Two solutions of benzyl (2R,3R)-3-isopropyl-1-tritylaziridine-2-carboxylate (200 mg, 430 μmol) and Pd/C (100 mg) in THF (4 mL) were stirred for 1 h at room temperature under H₂ atmosphere. The reaction mixtures were combined, filtered, and concentrated under reduced pressure. Purification by silica gel chromatography (0→50% EtOAc/pet. ether) afforded product (160 mg, 51% yield).

### Intermediate 8—Synthesis of (2S,3S)-1-benzyl-3-isopropylaziridine-2-carboxylic Acid

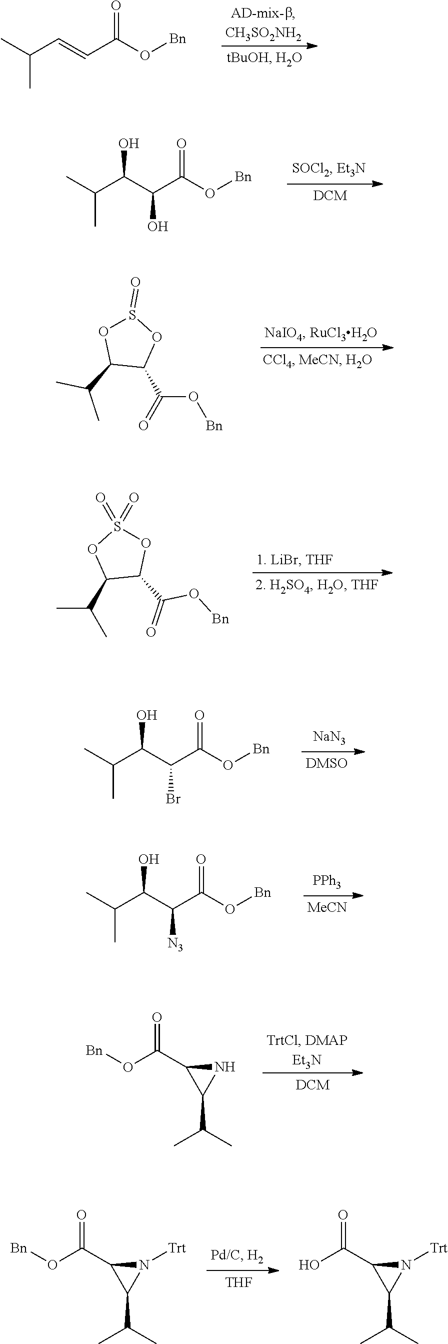

### Step 1: Synthesis of benzyl (2S,3R)-2,3-dihydroxy-4-methylpentanoate

To a solution of AD-mix-β (61.7 g) and methanesulfonamide (4.19 g, 44.1 mmol) in tert-BuOH (225 mL) and H₂O (225 mL) was added benzyl (E)-4-methylpent-2-enoate (9 g, 44.1 mmol). The mixture was stirred at room temperature for 12 h then Na₂SO₃ (67.5 g) was added and stirred for 30 min. The reaction mixture was diluted with EtOAc (300 mL) and H₂O (300 mL) and extracted into EtOAc (3×300 mL), washed with brine (300 mL), dried over Na₂SO₄, filtered, and concentrated under reduced pressure. Purification by silica gel chromatography (0→25% EtOAc/pet. ether) afforded product (8.8 g, 84% yield). LCMS (ESI) m/z: [M+Na] calcd for C₁₃H₁₈O₄: 261.11; found 261.0.

### Step 2: Synthesis of benzyl (4S,5R)-5-isopropyl-1,3,2-dioxathiolane-4-carboxylate 2-oxide

To a solution of benzyl (2S,3R)-2,3-dihydroxy-4-methylpentanoate (11.6 g, 48.7 mmol) in DCM (116 mL) at 0° C. was added Et₃N (20.3 mL, 146 mmol) and SOCl₂ (4.94 mL, 68.2 mmol). The reaction mixture was stirred 30 min then was diluted with DCM (100 mL) and H₂O (100 mL), extracted into DCM (3×100 mL), washed with brine (200 mL), dried over Na₂SO₄, filtered, and concentrated under reduced pressure which afforded product (13.0 g, 94% yield).

### Step 3: Synthesis of benzyl (4S,5R)-5-isopropyl-1,3,2-dioxathiolane-4-carboxylate 2,2-dioxide

To a solution of benzyl (4S,5R)-5-isopropyl-1,3,2-dioxathiolane-4-carboxylate 2-oxide (13 g, 45.7 mmol) in H₂O (290 mL), MeCN (145 mL), and CCl₄ (145 mL) was added NaIO₄ (3.80 mL, 68.6 mmol) and RuCl₃.H₂O (1.03 g, 4.57 mmol). The mixture was stirred at room temperature for 1 h then was diluted with DCM (500 mL) and H₂O (300 mL), filtered, and the filtrate was extracted into DCM (3×200 mL). The combined organic layers were washed sequentially with brine (500 mL) and sat. aq. Na₂CO₃ (300 mL), dried over Na₂SO₄, filtered, and concentrated under reduced pressure. Purification by silica gel chromatography (0→17% EtOAc/pet. ether) afforded product (11.5 g, 80% yield).

### Step 4: Synthesis of benzyl (2R,3R)-2-bromo-3-hydroxy-4-methylpentanoate

To a solution of benzyl (4S,5R)-5-isopropyl-1,3,2-dioxathiolane-4-carboxylate 2,2-dioxide (11.5 g, 38.3 mmol) in THF (520 mL) was added LiBr (3.65 mL, 146 mmol). The reaction mixture was stirred at room temperature for 5 h and then concentrated under reduced pressure. The residue was diluted in THF (130 mL) and H₂O (65 mL), cooled to 0° C., then H₂SO₄ solution (20% aq., 1.3 L) was added and the mixture was warmed to room temperature and stirred for 24 h. The mixture was diluted with EtOAc (1.0 L), washed with Na₂CO₃ (sat. aq., 300 mL), then was concentrated under reduced pressure. Purification by silica gel chromatography (0→17% EtOAc/pet. ether) afforded product (10 g, 83% yield).

### Step 5: Synthesis of benzyl (2S,3R)-2-azido-3-hydroxy-4-methylpentanoate

To a solution of benzyl (2R,3R)-2-bromo-3-hydroxy-4-methylpentanoate (10 g, 33.2 mmol) in DMSO (100 mL) was added NaN₃ (4.33 g, 66.6 mmol). The reaction mixture was stirred at room temperature for 12 h then was diluted with EtOAc (300 mL) and H₂O (200 mL). The mixture was extracted into EtOAc (2×200 mL), dried over Na₂SO₄, filtered, and concentrated under reduced pressure. Purification by silica gel chromatography (0→17% EtOAc/pet. ether) afforded product (7.5 g, 76% yield).

### Step 6: Synthesis of benzyl (2S,3S)-3-isopropylaziridine-2-carboxylate

To a solution of benzyl (2S,3R)-2-azido-3-hydroxy-4-methylpentanoate (7.5 g, 28.5 mmol) in MeCN (150 mL) was added PPh₃ (7.70 g, 29.3 mmol). The reaction mixture was stirred at room temperature for 1 h and then heated to 70° C. and stirred for 3 h. The reaction mixture was concentrated under reduced pressure and purification by silica gel chromatography (0→17% EtOAc/pet. ether) afforded product (4.5 g, 64% yield). LCMS (ESI) m/z: [M+H] calcd for C₁₃H₁₇NO₂: 220.13; found 220.1.

### Step 7: Synthesis of benzyl (2S,3S)-1-benzyl-3-isopropylaziridine-2-carboxylate

To a solution of benzyl (2S,3S)-3-isopropylaziridine-2-carboxylate (1 g, 4.56 mmol) in MeCN (10 mL) was added K₂CO₃ (3.15 g, 22.8 mmol) and benzyl bromide (812 μL, 6.84 mmol). The reaction mixture was stirred at room temperature for 6 h then was diluted with EtOAc (30 mL) and H₂O (30 mL), extracted into EtOAc (2×30 mL), washed with brine (50 mL), dried over Na₂SO₄, filtered, and concentrated under reduced pressure. Purification by silica gel chromatography (0→17% EtOAc/pet. ether) afforded product (1.3 g, 89% yield). LCMS (ESI) m/z: [M+H] calcd for C₂₀H₂₃NO₂: 310.18; found 310.1.

### Step 8: Synthesis of (2S,3S)-1-benzyl-3-isopropylaziridine-2-carboxylic Acid

To a solution of benzyl (2S,3S)-1-benzyl-3-isopropylaziridine-2-carboxylate (600 mg, 1.94 mmol) in THF (6 mL), MeCN (3 mL), and H₂O (6 mL) at 0° C. was added LiOH.H₂O (163 mg, 3.88 mmol). The reaction mixture was stirred at room temperature for 1 h and was adjusted to pH=7-8 with HCl (0.5M). Lyophilization afforded product (750 mg, crude). LCMS (ESI) m/z: [M+H] calcd for C₁₃H₁₇NO₂: 220.13; found 220.1.

### Intermediate 9—Synthesis of (2R,3R)-1-((R)-tert-butylsulfinyl)-3-cyclopropylaziridine-2-carboxylic Acid

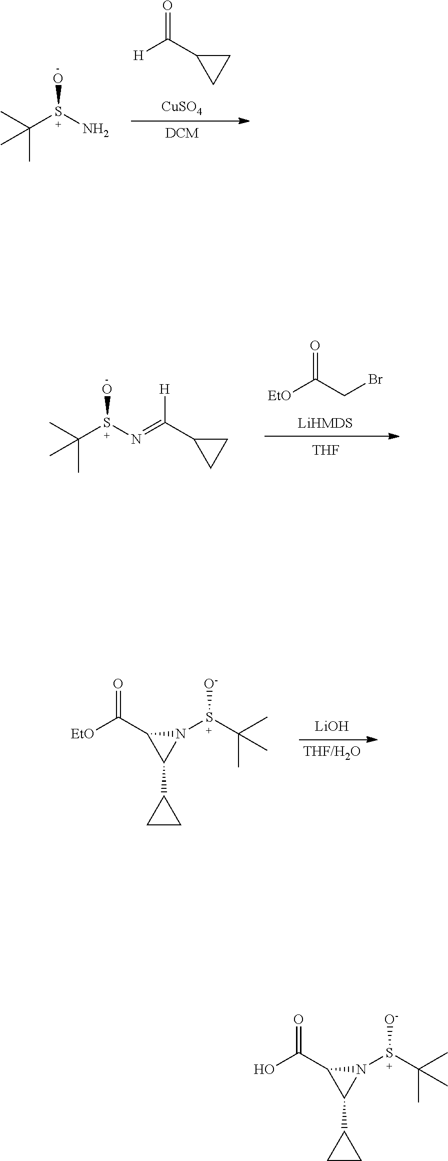

### Step 1: Synthesis of (R,E)-N-(cyclopropylmethylene)-2-methylpropane-2-sulfinamide

To a solution of (R)-2-methylpropane-2-sulfinamide (1.0 g, 8.25 mmol) and cyclopropanecarbaldehyde (1.16 g, 16.55 mmol) in DCM (50 mL) at room temperature was added CuSO₄ (3.95 g, 24.75 mmol). The resulting mixture was stirred overnight. The reaction mixture was then filtered, the filter cake washed with EtOAc, and the filtrate was concentrated under reduced pressure. The residue was purified by prep-TLC (17% EtOAc/pet. ether) to afford the desired product (1.4 g, 97.9% yield). LCMS (ESI) m/z: [M+H] calcd for C₈H₁₅NOS: 174.10; found 174.1.

### Step 2: Synthesis of ethyl (2R,3R)-1-((R)-tert-butylsulfinyl)-3-cyclopropylaziridine-2-carboxylate

To a solution of 1M LiHMDS (23 mL, 23 mmol) in THF (50.0 mL) at −78° C. was added ethyl bromoacetate (3.83 g, 22.95 mmol). The resulting mixture was warmed to −70° C. and stirred for 1 h. To the reaction mixture was then added (R,E)-N-(cyclopropylmethylene)-2-methylpropane-2-sulfinamide (2.0 g, 11.48 mmol). The resulting mixture was stirred for 1 h at −70° C. The reaction mixture was warmed to 0° C. and quenched with H₂O. The aqueous layer was extracted with EtOAc (3×100 mL). The combined organic layers were washed with brine, dried over Na₂SO₄, filtered, and concentrated under reduced pressure. The residue was purified by prep-TLC (25% EtOAc/pet. ether) to afford the desired product (1.8 g, 60.5% yield). LCMS (ESI) m/z: [M+H] calcd for C₁₂H₂₁NO₃S: 306.14; found 260.13.

### Step 3: Synthesis of (2R,3R)-1-((R)-tert-butylsulfinyl)-3-cyclopropylaziridine-2-carboxylic Acid

To a solution of ethyl (2R,3R)-1-((R)-tert-butylsulfinyl)-3-cyclopropylaziridine-2-carboxylate (900.0 mg, 3.47 mmol) in THF (3.0 mL) and H₂O (3.0 mL) at 0° C. was added LiOH.H₂O (218.4 mg, 5.21 mmol). The resulting mixture was stirred for 1 h and was then quenched by H₂O. The aqueous layer was extracted with EtOAc (3×50) and the combined organic layers were washed with brine, dried over Na₂SO₄, filtered, and concentrated under reduced pressure to afford the desired crude product (400 mg, 29.9% yield). LCMS (ESI) m/z: [M+H] calcd for C₁₀H₁₇NO₃S: 232.10; found 232.1.

### Intermediate 10—Synthesis of (2S,3S)-1-(tert-butylsufinyl)-3-cyclopropylaziridine-2-carboxylic Acid

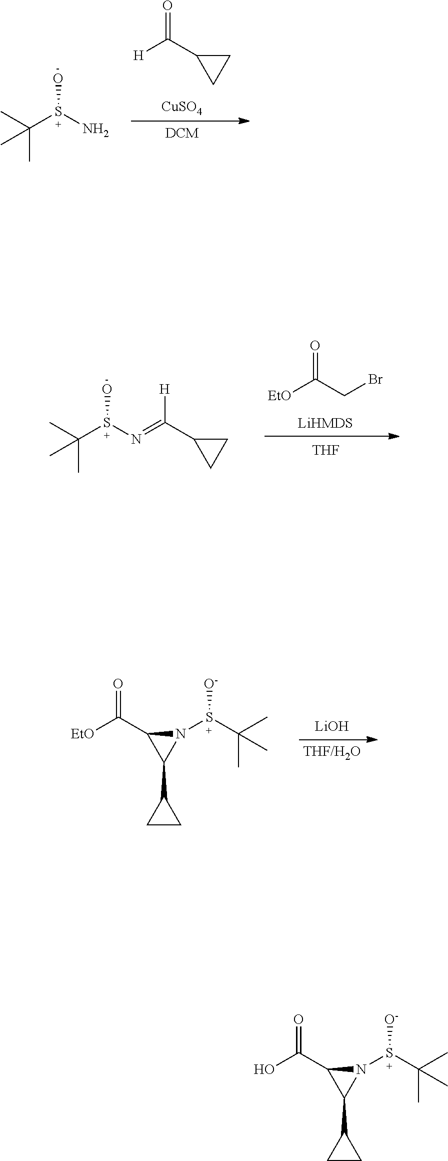

### Step 1: Synthesis of (E)-N-(cyclopropylmethylene)-2-methylpropane-2-sulfinamide

To a suspension of (S)-2-methylpropane-2-sulfinamide (4.0 g, 33.0 mmol) and CuSO₄ (15.80 g, 99.01 mmol) in DCM (200.0 mL) was added cyclopropanecarbaldehyde (4.63 g, 66.0 mmol). The resulting mixture was stirred overnight and was then filtered, the filter cake was washed with DCM (3×100 mL), and the filtrate was concentrated under reduced pressure to afford the desired product (3.5 g, 61.2% yield). LCMS (ESI) m/z: [M+H] calcd for C₈H₁₅NOS: 174.10; found 174.1.

### Step 2: Synthesis of ethyl (2S,3S)-1-(tert-butylsulfinyl)-3-cyclopropylaziridine-2-carboxylate

To a solution of ethyl bromoacetate (481.91 mg, 2.886 mmol) in THF (5.0 mL) at −78° C. was added LiHMDS (2.90 mL, 2.90 mmol). The resulting mixture was stirred for 2 h at −78° C. and then a solution of (E)-N-(cyclopropylmethylene)-2-methylpropane-2-sulfinamide (250.0 mg, 1.443 mmol) was added. The resulting mixture was stirred for 2 h at −78° C. and was then was then quenched with H₂O at 0° C. The aqueous layer was extracted with EtOAc (3×50 mL), and the combined organic layers were dried over Na₂SO₄, filtered, and concentrated under reduced pressure. The residue was purified by prep-TLC (17% EtOAc/pet. ether) to afford the desired product (250 mg, 66.8% yield). LCMS (ESI) m/z: [M+H] calcd for C₁₂H₂₁NO₃S: 260.13; found 260.1.

### Step 3: Synthesis of (2S,3S)-1-(tert-butylsulfinyl)-3-cyclopropylaziridine-2-carboxylic Acid

A solution of ethyl (2S,3S)-1-(tert-butylsulfinyl)-3-cyclopropylaziridine-2-carboxylate (500.0 mg, 1.928 mmol) in THF (2.0 mL) and H₂O (2.0 mL) at 0° C. was added LiOH.H₂O (121.34 mg, 2.89 mmol). The reaction mixture was stirred for 1 h and was then acidified to pH 6 with 1 M HCl (aq.). The resulting mixture was extracted with EtOAc (2×10 mL) and the combined organic layers were washed with brine (10 mL), dried over Na₂SO₄, filtered, and the filtrate was concentrated under reduced pressure to afford the desired product (400 mg, 89.7% yield). LCMS (ESI) m/z: [M+H] calcd for C₁₀H₁₇NO₃S: 232.10; found 232.0.

### Intermediate 11—Synthesis of (2R,3S)-3-cyclopropylaziridine-2-carboxylic Acid

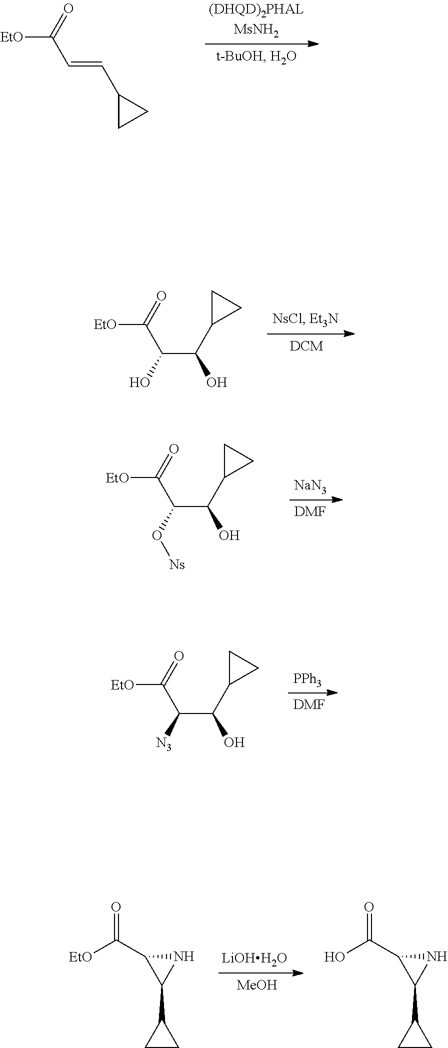

### Step 1: Synthesis of ethyl (2S,3R)-3-cyclopropyl-2,3-dihydroxypropanoate

A solution of ethyl (E)-3-cyclopropylacrylate (10.4 mL, 71 mmol) in tert-BuOH (270 mL) and H₂O (270 mL) was stirred at 0° C. After 5 min MsNH₂ (6.8 g, 71 mmol) and (DHQD)₂PHAL (100 g, 130 mmol) were added and the reaction mixture was warmed to room temperature. After stirring overnight, sat. Na₂SO₃ was added and the mixture was stirred for 30 min. The mixture was acidified to pH 6 with KH₂PO₄. Purification by silica gel column chromatography (33% EtOAC/pet. ether) afforded desired product (5.5 g, 44% yield).

### Step 2: Synthesis of ethyl (2S,3R)-3-cyclopropyl-3-hydroxy-2-(((4-nitrophenyl)sulfonyl)oxy)propanoate

A solution of ethyl (2S,3R)-3-cyclopropyl-2,3-dihydroxypropanoate (5.40 g, 31.0 mmol) and Et₃N (13.0 mL, 93.0 mmol) in DCM (20 mL) was stirred at 0° C. and a solution of 4-nitrobenzenesulfonyl chloride (6.53 g, 29.5 mmol) in DCM (10 mL) was added. The reaction mixture was stirred for 1.5 h and was then extracted with DCM (3×200 mL). The combined organic layers were washed with brine (100 mL), dried with Na₂SO₄, filtered, and concentrated under reduced pressure. Purification by silica gel column chromatography (33% EtOAc/pet. ether) afforded desired product (6.9 g, 62% yield).

### Step 3: Synthesis of ethyl (2R,3R)-2-azido-3-cyclopropyl-3-hydroxypropanoate

A mixture of ethyl (2S,3R)-3-cyclopropyl-3-hydroxy-2-(((4-nitrophenylsulfonyloxy)propanoate (6.90 g, 19.2 mmol) and NaN₃ (6.24 g, 96.0 mmol) in DMF (70.0 mL) was heated to 50° C. The reaction mixture was stirred for 5 h and then extracted with EtOAc (3×200 mL). The combined organic layers were washed with brine (100 mL), dried with Na₂SO₄, filtered, and concentrated under reduced pressure. Purification by silica gel column chromatography (20% EtOAc/pet. ether) afforded desired product (2.8 g, 73% yield).

### Step 4: Synthesis of ethyl (2R,3S)-3-cyclopropylaziridine-2-carboxylate

A mixture of triphenylphosphine (1.84 g, 7.02 mmol) in DMF (5 mL) was stirred at 0° C. After 5 min ethyl (2R,3R)-2-azido-3-cyclopropyl-3-hydroxypropanoate (1.40 g, 7.03 mmol) was added and the reaction was warmed to room temperature. The reaction mixture was heated to 80° C. and stirred for 1 h. The mixture was then cooled to room temperature and extracted with EtOAc (3×50 mL). The combined organic layers were washed with brine (50 mL), dried with Na₂SO₄, filtered, and concentrated under reduced pressure. Purification by silica gel column chromatography (20% EtOAc/pet. ether) afforded the desired product (230 mg, 46% yield). LCMS (ESI) m/z: [M+H] calcd for C₈H₁₃NO₂: 156.10; found 156.2.

### Step 5: Synthesis of Lithium (2R,3S)-3-cyclopropylaziridine-2-carboxylate

To a mixture of ethyl (2R,3S)-3-cyclopropylaziridine-2-carboxylate (230 mg, 1.5 mmol) in MeOH (3.0 mL) was added LiOH.H₂O (125 mg, 3.0 mmol). The reaction was stirred for 3 h and then filtered. The filtrate was concentrated under reduced pressure which afforded the desired product (150 mg, crude). LCMS (ESI) m/z: [M+H] calcd for C₆H₉NO₂: 128.07; found 128.2.

### Intermediate 12—Synthesis of (2S,3R)-3-cyclopropylaziridine-2-carboxylic Acid

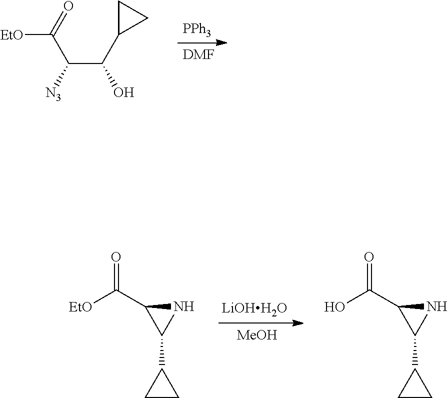

### Step 1: Synthesis of ethyl (2S,3R)-3-cyclopropylaziridine-2-carboxylate

A mixture of PPh₃ (1.4 g, 5.4 mmol) in DMF (15.0 mL) was stirred at 0° C. After 30 min, ethyl (2S,3S)-2-azido-3-cyclopropyl-3-hydroxypropanoate (980 mg, 4.92 mmol) was added. The reaction mixture was heated to 80° C. After 2 h the reaction was quenched by the addition of H₂O (20 mL) and was extracted with EtOAc (3×30 mL). Purification by silica gel column chromatography (17% EtOAc/pet. ether) afforded desired product (500 mg, 65% yield).

### Step 2: Synthesis of Lithium (2S,3R)-3-cyclopropylaziridine-2-carboxylate

To a solution of ethyl (2S,3R)-3-cyclopropylaziridine-2-carboxylate (450 mg, 2.9 mmol) in THF (6.0 mL) and H₂O (2.0 mL) was added LiOH (90 mg, 3.8 mmol). The reaction was stirred for 2 h and then filtered. The filtrate was concentrated under reduced pressure which afforded the desired product (300 mg, crude).

### Intermediate 13—Synthesis of (2S,3S)-1-((S)-tertbutylsulfinyl)-3-cyclobutylaziridine-2-carboxylic Acid

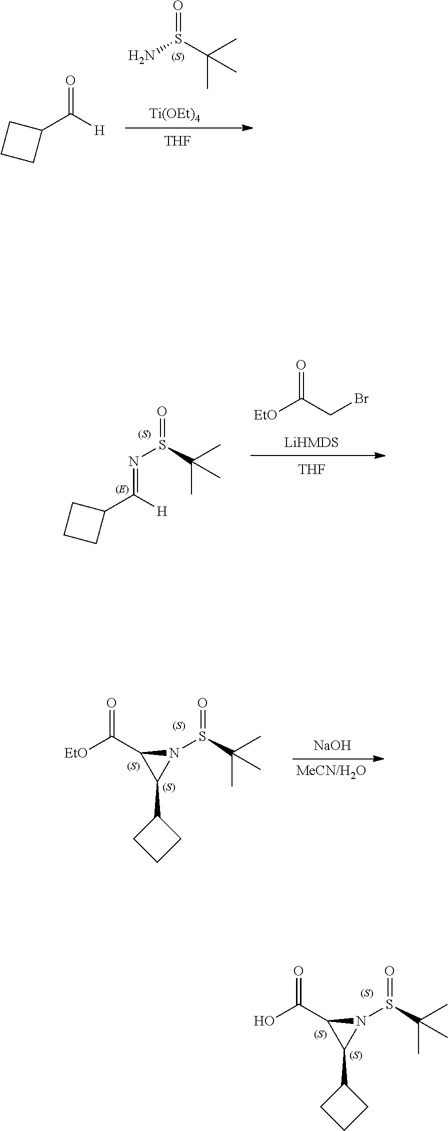

### Step 1: Synthesis of (S,E)-N-(cyclobutylmethylene)-2-methylpropane-2-sulfinamide

To a solution of cyclobutanecarbaldehyde (0.5 g, 5.94 mmol) in THF (10 mL) was added (S)-2-methylpropane-2-sulfinamide (792.48 mg, 6.54 mmol) and Ti(OEt)₄ (2.47 mL, 11.89 mmol). The mixture was stirred at 75° C. for 3 h. The reaction mixture was cooled to room temperature and quenched by addition brine (30 mL), and filtered to remove solids. The mixture was extracted with EtOAc (3×30 mL). The combined organic layers were washed with brine (2×10 mL), dried over Na₂SO₄, filtered and concentrated under reduced pressure to give a residue. The residue was purified by silica gel chromatography (2%→10% EtOAc/pet. ether) to afford the desired product (907.3 mg, 39.9% yield). LCMS (ESI) m/z: [M+H] calcd for C₉H₁₇NOS: 188.1; found 188.3.

### Step 2: Synthesis of ethyl (2S,3S)-1-((S)-tert-butylsulfinyl)-3-cyclobutylaziridine-2-carboxylate

To a solution of ethyl 2-bromoacetate (1.60 g, 9.61 mmol, 1.06 mL) in THF (9 mL) was added LiHMDS (1 M, 9.61 mL) at −78° C., after 2 min, (S,E)-N-(cyclobutylmethylene)-2-methylpropane-2-sulfinamide (0.9 g, 4.81 mmol) was added. The mixture was stirred at −78° C. for 2 h. The reaction mixture was quenched by addition H₂O (25 mL) at −78° C. and warmed to room temperature, then the mixture extracted with EtOAc (3×20 mL). The combined organic layers were washed with brine (2×5 mL), dried over Na₂SO₄, filtered and concentrated under reduced pressure to give a residue, which was purified by silica gel chromatography (10%→20% EtOAc/pet. ether) to afford the desired product (426 mg, crude). LCMS (ESI) m/z: [M+H] calcd for C₁₃H₂₃NO₃S: 274.14; found 274.3.

### Step 3: Synthesis of (2S,3S)-1-((S)-tert-butylsulfinyl)-3-cyclobutylaziridine-2-carboxylic Acid

To a solution of (2S,3S)-1-((S)-tert-butylsulfinyl)-3-cyclobutylaziridine-2-carboxylate (100 mg, 365.78 μmol) in MeCN (0.5 mL) and H₂O (0.5 mL) was added NaOH (21.95 mg, 548.67 μmol) at 0° C., the mixture was warmed to room temperature and stirred for 2 h. The reaction mixture was adjusted to pH 5 by addition aq. 10% citric acid (˜10 mL) and was then extracted with EtOAc (3×20 mL). The combined organic layers were washed with brine (2×5 mL), dried over Na₂SO₄, filtered and concentrated under reduced pressure to afford the desired product (92.6 mg, crude). LCMS (ESI) m/z: [M+H] calcd for C₁₁H₁₉NO₃S: 246.11; found 246.3.

### Intermediate 14—Synthesis of (2R,3R)-1-((R)-tert-butylsulfinyl)-3-cyclobutylaziridine-2-carboxylic Acid

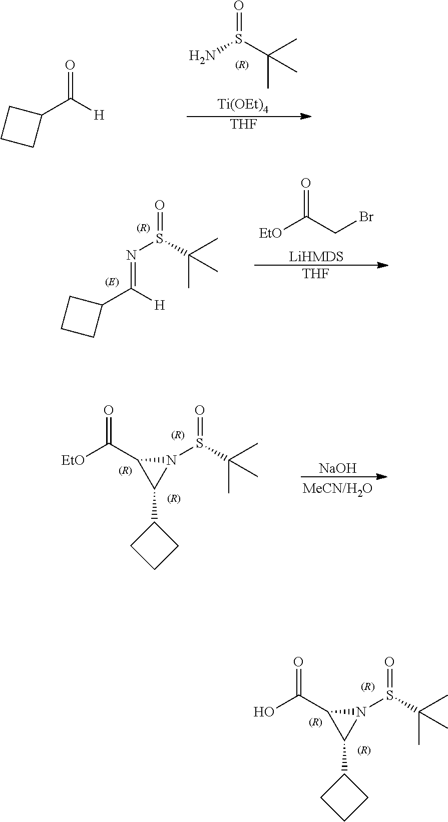

### Step 1: Synthesis of (R,E)-N-(cyclobutylmethylene)-2-methylpropane-2-sulfinamide

To a solution of cyclobutanecarbaldehyde (0.25 g, 2.97 mmol) in THF (5 mL) was added (R)-2-methylpropane-2-sulfinamide (396.24 mg, 3.27 mmol) and Ti(OEt)₄ (1.36 g, 5.94 mmol, 1.23 mL). The mixture was stirred at 75° C. for 3 h in two batches. The two batches were combined and the reaction mixture was quenched by the addition of brine (15 mL). The solution was extracted with EtOAc (3×20 mL) and the combined organic layers were washed with brine (2×5 mL), dried over Na₂SO₄, filtered and concentrated under reduced pressure to give a residue, which was purified by silica gel chromatography (10%→20% EtOAc/pet. ether) to afford the desired product (786.7 mg, 70.7% yield). LCMS (ESI) m/z: [M+H] calcd for C₉H₁₇NOS: 188.1; found 188.3.

### Step 2: Synthesis of ethyl (2R,3R)-1-((R)-tert-butylsulfinyl)-3-cyclobutylaziridine-2-carboxylate

To a solution of ethyl 2-bromoacetate (236.19 μL, 2.14 mmol) in THF (2 mL) was added LIHMDS (1 M, 2.14 mL) at −78° C., after 30 min, (R,E)-N-(cyclobutylmethylene)-2-methylpropane-2-sulfinamide (0.2 g, 1.07 mmol) was added. The mixture was warmed to −40° C. and stirred for 4 h. The reaction mixture was quenched by addition H₂O (18 mL) at −40° C. and warmed to room temperature The mixture was extracted with EtOAc (3×15 mL) and the combined organic layers were washed with brine (2×5 mL), dried over Na₂SO₄, filtered and concentrated under reduced pressure to give a residue, which was purified by prep-TLC (20% EtOAc/pet. ether) to afford the desired product (0.1 g, crude). LCMS (ESI) m/z: [M+H] calcd for C₁₃H₂₃NO₃S: 274.14; found 274.3.

### Step 3: Synthesis of (2R,3R)-1-((R)-tert-butylsulfinyl)-3-cyclobutylaziridine-2-carboxylic Acid

In two batches, to a solution ethyl (2R,3R)-1-((R)-tert-butylsulfinyl)-3-cyclobutylaziridine-2-carboxylate (25 mg, 91.44 μmol) in MeCN (0.25 mL) and H₂O (0.25 mL) was added NaOH (5.49 mg, 137.17 μmol) at 0° C., the mixture was warmed to room temperature and stirred for 5 h. The reaction mixtures were combined, and adjust to pH to 5 with aq. 10% citric acid (10 mL), then extracted with EtOAc (3×20 mL). The combined organic layers were washed with brine (2×5 mL), dried over Na₂SO₄, filtered and concentrated under reduced pressure to afford the desired product (53 mg, crude). LCMS (ESI) m/z: [M+H] calcd for C₁₁H₁₉NO₃S: 246.11; found 246.2.

### Intermediate 15, 16, 17, and 18—Synthesis of ethyl (2R,3R)-1-benzhydryl-3-(oxetan-3-yl)aziridine-2-carboxylate (15), ethyl (2S,3S)-1-benzhydryl-3-(oxetan-3-yl)aziridine-2-carboxylate (16), ethyl (2R,3S)-1-benzhydryl-3-(oxetan-3-yl)aziridine-2-carboxylate (17), and ethyl (2S,3R)-1-benzhydryl-3-(oxetan-3-yl)aziridine-2-carboxylate (18)

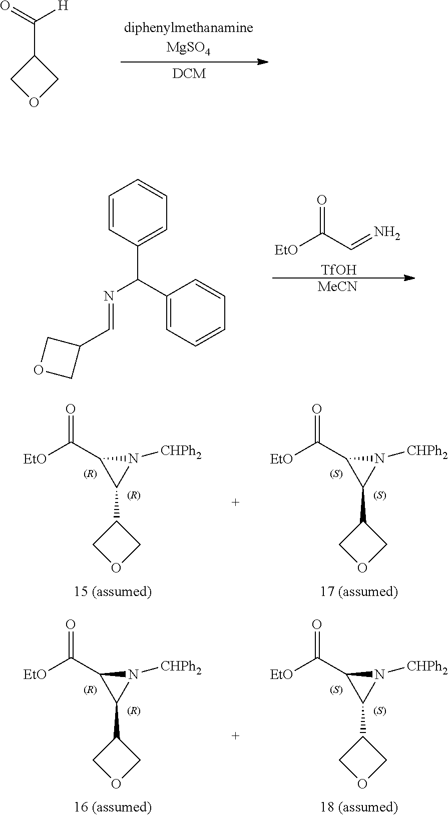 [0850] Intermediates 15, 16, 17, and 18 are used to make Intermediates 19, 20, 21, and 22 below

### Step 1: Synthesis of N-benzhydryl-1-(oxetan-3-yl)methanimine

To a solution oxetane-3-carbaldehyde (5.0 g, 58 mmol) and MgSO₄ (6.99 g, 58.1 mmol) in DCM (120 mL) at 0° C. was added diphenylmethanamine (12.1 mL, 69.7 mmol). The mixture was stirred for 12 h at room temperature then filtered and concentrated under reduced pressure to afford the desired compound (14 g, 95.9% yield) which was used without further purification.

### Step 2: Synthesis of ethyl cis-1-benzhydryl-3-(oxetan-3-yl)aziridine-2-carboxylate and ethyl trans-1-benzhydryl-3-(oxetan-3-yl)aziridine-2-carboxylate

To a solution of N-benzhydryl-1-(oxetan-3-yl)methanimine (10 g, 39.79 mmol) in MeCN (150 mL) was added TfOH (878 mL, 9.95 mmol) and after 5 min ethyl diazoacetate (5.0 mL, 47.8 mmol) was added. The reaction mixture was stirred for 12 h at room temperature then cooled to 0° C. and quenched by the addition of saturated NaHCO₃ (300 mL). The aqueous layer was extracted with EtOAc (3×200 mL) and the combined organic layers were washed with brine, dried with Na₂SO₄, filtered, and concentrated under reduced pressure. Purification by reverse phase chromatography (50→65% MeCN/H₂O, 10 mM NH₄HCO₃) afforded racemic ethyl cis-1-benzhydryl-3-(oxetan-3-yl)aziridine-2-carboxylate (1.1 g, 8.2% yield) and racemic ethyl trans-1-benzhydryl-3-(oxetan-3-yl)aziridine-2-carboxylate (780 mg, 5.8% yield)

### Step 3: Separation of Racemic ethyl cis-1-benzhydryl-3-(oxetan-3-yl)aziridine-2-carboxylate: ethyl (2R,3R)-1-benzhydryl-3-(oxetan-3-yl)aziridine-2-carboxylate and ethyl (2S,3S)-1-benzhydryl-3-(oxetan-3-yl)aziridine-2-carboxylate

Racemic ethyl cis-1-benzhydryl-3-(oxetan-3-yl)aziridine-2-carboxylate (800 mg, 2.37 mmol) was separated by chiral prep-SFC (25% MeOH/CO₂) to afford ethyl (2R,3R)-1-benzhydryl-3-(oxetan-3-yl) aziridine-2-carboxylate (320 mg, 40% yield) and ethyl (2S,3S)-1-benzhydryl-3-(oxetan-3-yl)aziridine-2-carboxylate (320 mg, 40% yield).

### Step 4: Separation of Racemic ethyl trans-1-benzhydryl-3-(oxetan-3-yl)aziridine-2-carboxylate: ethyl (2R,3S)-1-benzhydryl-3-(oxetan-3-yl)aziridine-2-carboxylate and ethyl (2S,3R)-1-benzhydryl-3-(oxetan-3-yl)aziridine-2-carboxylate

Racemic ethyl trans1-benzhydryl-3-(oxetan-3-yl)aziridine-2-carboxylate (700 mg, 2.07 mmol) was separated by chiral prep-SFC (25% EtOH, 0.1% NH₄OH/CO₂) to afford ethyl (2R,3S)-1-benzhydryl-3-(oxetan-3-yl) aziridine-2-carboxylate (300 mg, 42% yield) and ethyl (2S,3R)-1-benzhydryl-3-(oxetan-3-yl)aziridine-2-carboxylate (320 mg, 43% yield).

### Intermediate 19 and 20—Synthesis of (2R,3R)-1-benzhydryl-3-(oxetan-3-yl)aziridine-2-carboxylic Acid (19) and (2S,3S)-1-benzhydryl-3-(oxetan-3-yl)aziridine-2-carboxylic Acid (20)

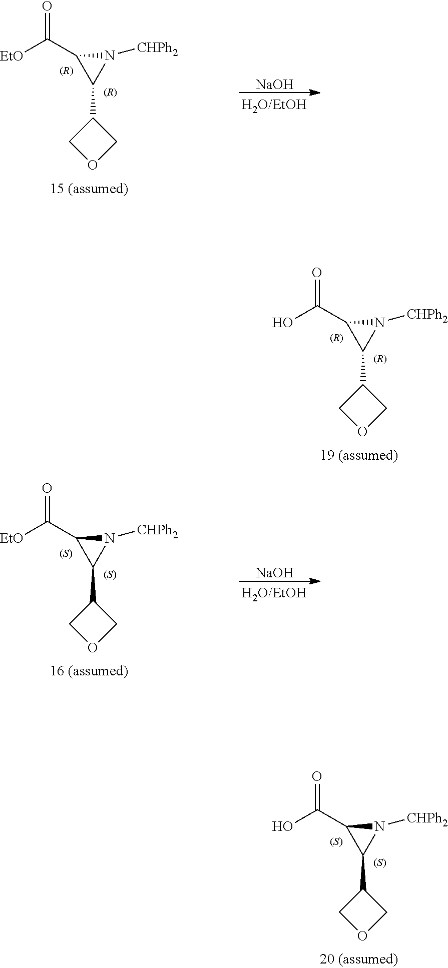 [0856] Intermediates 19 and 20 are derived from Intermediates 15 and 16 above

### Step 1: Synthesis of (2R,3R)-1-benzhydryl-3-(oxetan-3-yl)aziridine-2-carboxylic Acid (19)

To a solution of ethyl (2R,3R)-1-benzhydryl-3-(oxetan-3-yl)aziridine-2-carboxylate (15) (156 mg, 463 mmol) in EtOH (3 mL) was added 2M NaOH (347 mL, 696 mmol). The reaction mixture was stirred for 3 h at room temperature and then concentrated under reduced pressure. The concentrate was acidified to pH 5 with 1M HCl and extracted with DCM (3×5 mL) and the combined organic layers were washed with brine, dried with Na₂SO₄, filtered and concentrated under reduced pressure to afford the desired compound (110 mg, 72.6% yield).

### Step 2: Synthesis of (2S,3S)-1-benzhydryl-3-(oxetan-3-yl)aziridine-2-carboxylic Acid (20)

To a solution of ethyl (2S,3S)-1-benzhydryl-3-(oxetan-3-yl)aziridine-2-carboxylate (16) (150 mg, 444 mmol) in EtOH (5 mL) was added 2M NaOH (333 mL, 666 mmol). The reaction mixture was stirred for 3 h at room temperature and then acidified to pH 5 with 1M HCl. The aqueous layer extracted with DCM (3×10 mL) and the combined organic layers were washed with brine, dried with Na₂SO₄, filtered, and concentrated under reduced pressure to afford the desired compound (120 mg, 86.1% yield).

### Intermediate 21 and 22—Synthesis of Sodium (2R,3S)-1-benzhydryl-3-(oxetan-3-yl)aziridine-2-carboxylate (21) and Sodium (2S,3R)-1-benzhydryl-3-(oxetan-3-yl)aziridine-2-carboxylate (22)

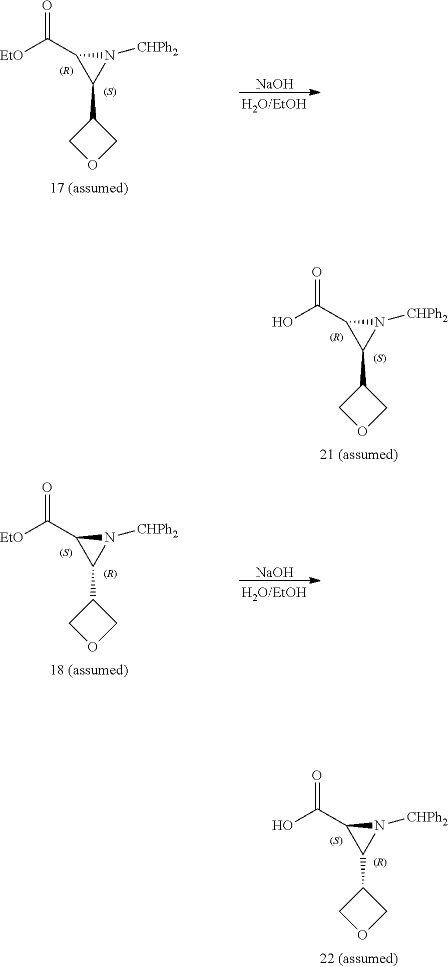 [0860] Intermediates 21 and 22 are derived from Intermediates 17 and 18 above

### Step 1: Synthesis of Sodium (2R,3S)-1-benzhydryl-3-(oxetan-3-yl)aziridine-2-carboxylate (21)

To a solution of ethyl (2R,3S)-1-benzhydryl-3-(oxetan-3-yl)aziridine-2-carboxylate (17) (150 mg, 444 mmol) in EtOH (3 mL) was added 2M NaOH (333.42 mL, 666 mmol). The reaction mixture was stirred for 3 h at room temperature and then the pH was adjusted to pH 8 with 1M HCl. The resulting solution was lyophilized to afford the desired compound (165 mg, crude) which was used without further purification. LCMS (ESI) m/z: [M] calcd for C₁₉H₁₈NO₃: 308.13; found 308.0.

### Step 2: Synthesis of Sodium (2S,3R)-1-benzhydryl-3-(oxetan-3-yl)aziridine-2-carboxylate (22)

To a solution of ethyl (2S,3R)-1-benzhydryl-3-(oxetan-3-yl)aziridine-2-carboxylate (18) (170 mg, 503 mmol) in EtOH (3 mL) was added 2M NaOH (378 mL, 754 mmol). The reaction mixture was stirred for 3 h at room temperature and then the pH was adjusted to pH 8 with 1M HCl. The resulting solution was lyophilized to afford the desired compound (230 mg, crude) which was used without further purification. LCMS (ESI) m/z: [M] calcd for C₁₉H₁₈NO₃: 308.13; found 308.0.

### Intermediate 23—Synthesis of (2R,3S)-1-((R)-tert-butylsulfinyl)-3-(methoxycarbonyl)aziridine-2-carboxylic Acid

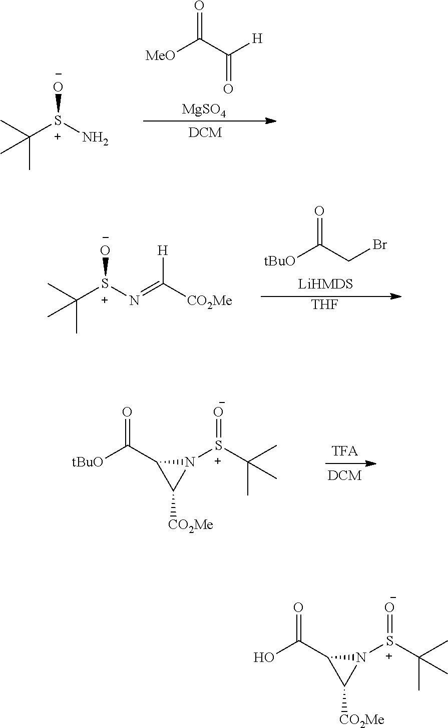

### Step 1: Synthesis of methyl (R,E)-2-((tert-butylsulfinyl)imino)acetate

To a solution of (R)-2-methylpropane-2-sulfinamide (13.21 g, 109.01 mmol) and methyl 2-oxoacetate (8.0 g, 90.85 mmol) in DCM (130 mL) at room temperature was added MgSO₄ (54.67 g, 454.23 mmol). The resulting mixture was heated to 35° C. and stirred for 16 h. The resulting mixture was filtered, the filter cake washed with EtOAc (3×50 mL), and the filtrate was concentrated under reduced pressure. The residue was purified by normal phase chromatography (25% EtOAc/pet. ether) to afford the desired (5.8 g, 33.4% yield). LCMS (ESI) m/z: [M+H] calcd for C₇H₁₃NO₃S: 192.07; found 191.9.

### Step 2: Synthesis of 2-(tert-butyl) 3-methyl (2R,3S)-1-((R)-tert-butylsulfinyl)aziridine-2,3-dicarboxylate

To a solution of 1M LiHMDS (61.40 mL, 61.40 mmol) in THF (300.0 mL) at −78° C. was added tert-butyl 2-bromoacetate (11.83 g, 60.65 mmol). The resulting mixture was stirred for 30 min. To the reaction mixture was then added methyl methyl (R,E)-2-((tert-butylsulfinyl)imino)acetate (5.8 g, 30.33 mmol). The resulting mixture was warmed to −60° C. and stirred for 2.5 h. The reaction was warmed to 0° C. and quenched with sat. NH₄Cl (aq.). The resulting mixture was extracted with EtOAc (3×200 mL). The combined organic layers were washed with brine (500 mL), dried over Na₂SO₄, filtered, and concentrated under reduced pressure. The residue was purified by reverse phase chromatography (10→50% MeCN/H₂O) to afford the desired product (1.34 g, 4.5% yield). LCMS (ESI) m/z: [M+H] calcd for C₁₃H₂₃NO₅S: 306.14; found 306.2.

### Step 3: Synthesis of (2R,3S)-1-((R)-tert-butylsulfinyl)-3-(methoxycarbonyl)aziridine-2-carboxylic Acid

To a solution of 2-(tert-butyl) 3-methyl (2R,3S)-1-((R)-tert-butylsulfinyl)aziridine-2,3-dicarboxylate (302.0 mg, 0.99 mmol) in DCM (3.0 mL) at 0° C. was added TFA (1.50 mL). The resulting mixture was stirred for 1 h and then concentrated under reduced pressure to afford the desired crude product (300 mg). LCMS (ESI) m/z: [M+H] calcd for C₉H₁₅NO₅S: 250.07; found 250.1.

### Intermediate 24—Synthesis of (2R,3S)-1-((S)-tert-butylsulfinyl)-3-(methoxycarbonyl)aziridine-2-carboxylic Acid

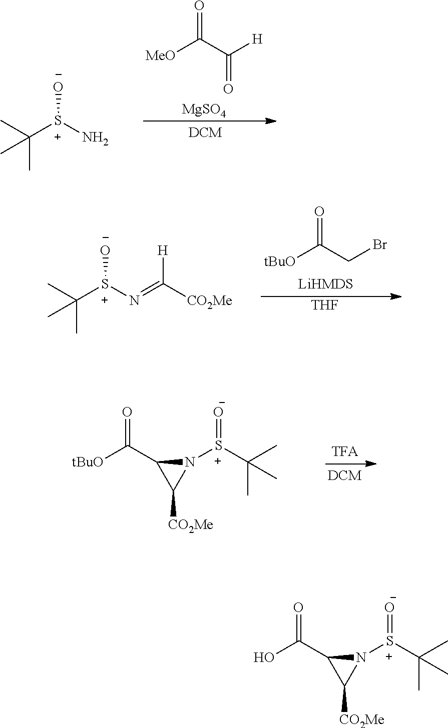

### Step 1: Synthesis of methyl (S,E)-2-((tert-butylsulfinyl)imino)acetate

To a solution of (S)-2-methylpropane-2-sulfinamide (9.81 g, 80.94 mmol) and methyl 2-oxoacetate (5.94 g, 67.45 mmol) in DCM (100 mL) at room temperature was added MgSO₄ (40.60 g, 337.26 mmol). The resulting mixture was heated to 35° C. and stirred for 16 h. The resulting mixture was filtered, the filter cake washed with EtOAc (3×50 mL), and the filtrate was concentrated under reduced pressure. The residue was purified by normal phase chromatography (25% EtOAc/pet. ether) to afford the desired (5.68 g, 44.0% yield). LCMS (ESI) m/z: [M+H] calcd for C₇H₁₃NO₃S: 192.07; found 191.1.

### Step 2: Synthesis of 2-(tert-butyl) 3-methyl (2R,3S)-1-((S)-tert-butylsulfinyl)aziridine-2,3-dicarboxylate

To a solution of 1M LiHMDS (59.40 mL, 59.40 mmol) in THF (300.0 mL) at −78° C. was added tert-butyl 2-bromoacetate (11.59 g, 59.40 mmol). The resulting mixture was stirred for 30 min. To the reaction mixture was then added methyl methyl (S,E)-2-((tert-butylsulfinyl)imino)acetate (5.68 g, 29.70 mmol). The resulting mixture was warmed to −60° C. and stirred for 2.5 h. The reaction was warmed to 0° C. and quenched with sat. NH₄Cl (aq.). The resulting mixture was extracted with EtOAc (3×200 mL). The combined organic layers were washed with brine (500 mL), dried over Na₂SO₄, filtered, and concentrated under reduced pressure. The residue was purified by reverse phase chromatography (10→50% MeCN/H₂O) to afford the desired product (1.26 g, 13.9% yield). LCMS (ESI) m/z: [M+H] calcd for C₁₃H₂₃NO₅S: 306.14; found 306.1.

### Step 3: Synthesis of (2R,3S)-1-((S)-tert-butylsulfinyl)-3-(methoxycarbonyl)aziridine-2-carboxylic Acid

To a solution of 2-(tert-butyl) 3-methyl (2R,3S)-1-((S)-tert-butylsulfinyl)aziridine-2,3-dicarboxylate (457.0 mg, 1.50 mmol) in DCM (6.0 mL) at 0° C. was added TFA (3.0 mL). The resulting mixture was stirred for 1 h and then concentrated under reduced pressure to afford the desired crude product (450 mg). LCMS (ESI) m/z: [M+H] calcd for C₉H₁₅NO₅S: 250.07; found 250.1.

### Intermediate 25 and 26—Synthesis of (2R,3S)-1-(4-methoxybenzyl)-3-(trifluoromethyl)aziridine-2-carboxylic Acid and (2S,3R)-1-(4-methoxybenzyl)-3-(trifluoromethyl)aziridine-2-carboxylic Acid

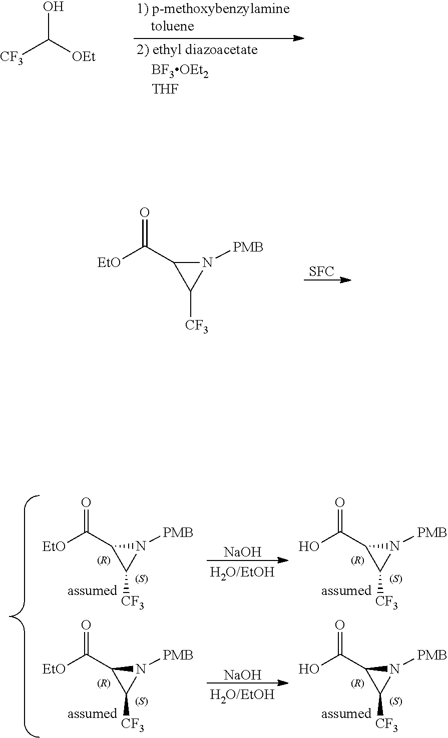

### Step 1: Synthesis of ethyl 1-(4-methoxybenzyl)-3-(trifluoromethyl)aziridine-2-carboxylate

A solution of 1-ethoxy-2,2,2-trifluoroethan-1-ol (2.17 mL, 18.37 mmol) and p-methoxybenzylamine (1.89 mL, 14.58 mmol) in toluene (46 ml) was refluxed for 16 h under Dean-Stark conditions. The reaction was concentrated under reduced pressure and the resulting residue was dissolved in THF (80 mL) and cooled to −78° C. BF₃.Et₂O (0.380 mL, 2.92 mmol) was added to the solution, followed by dropwise addition of ethyl diazoacetate (1.83 mL, 17.50 mmol). The reaction was stirred for 4 h at room temperature. The reaction mixture was quenched by addition of aq. sat. NaHCO₃ (5 mL), and the resulting solution was extracted with DCM (3×50 mL). The combined organic layers were washed with H₂O (20 mL) and brine (10 mL). The organic phase was dried over Na₂SO₄, filtered, and concentrated under reduced pressure. The residue was purified by silica gel column chromatography (1→10% EtOAc/pet. ether) afford the desired product (2 g, 45.2 yield).

### Step 2: Synthesis of ethyl (2R,3S)-1-(4-methoxybenzyl)-3-(trifluoromethyl)aziridine-2-carboxylate and ethyl (2S,3R)-1-(4-methoxybenzyl)-3-(trifluoromethyl)aziridine-2-carboxylate

Ethyl 1-(4-methoxybenzyl)-3-(trifluoromethyl)aziridine-2-carboxylate (1 g) was purified by SFC separation (column: REGIS(S,S)WHELK-O1 (250 mm*25 mm, 10 um); mobile phase: [Neu-IPA]; B %: 13%-13%, min) to afford ethyl (2R,3S)-1-(4-methoxybenzyl)-3-(trifluoromethyl)aziridine-2-carboxylate (530 mg) and ethyl (2S,3R)-1-(4-methoxybenzyl)-3-(trifluoromethyl)aziridine-2-carboxylate (470 mg).

### Step 3: Synthesis of (2R,3S)-1-(4-methoxybenzyl)-3-(trifluoromethyl)aziridine-2-carboxylic Acid

To a solution of ethyl (2R,3S)-1-(4-methoxybenzyl)-3-(trifluoromethyl)aziridine-2-carboxylate (430 mg, 1.42 mmol) in EtOH (4 ml) and H₂O (6 mL) was added NaOH (113.42 mg, 2.84 mmol). The mixture was stirred at room temperature for 5 h. The mixture was acidified with aq. HCl (2M) to pH=1-2. The reaction mixture was poured into H₂O (3 ml) and the aqueous phase was extracted with EtOAc (3×3 mL). The combined organic phase was washed with brine (5 mL), dried over Na₂SO₄, filtered, and concentrated under reduced pressure to afford the desired product (350 mg, 89.1% yield). LCMS (ESI) m/z: [M+H] calcd for C₁₂H₁₁FNO₃: 274.08; found 274.1

### Step 4: Synthesis of (2S,3R)-1-(4-methoxybenzyl)-3-(trifluoromethyl)aziridine-2-carboxylic Acid

To a solution of ethyl (2S,3R)-1-(4-methoxybenzyl)-3-(trifluoromethyl)aziridine-2-carboxylate (370 mg, 1.22 mmol) in H₂O (2 mL) and EtOH (4 mL) was added NaOH (97.59 mg, 2.44 mmol). The mixture was stirred at room temperature for 5 h. The mixture was brought to pH=1-2 with the addition of aq. HCl (2 M). The reaction mixture was poured into H₂O (3 mL) and the aqueous phase was extracted with EtOAc (3×3 mL). The combined organic phase was washed with brine (5 mL), dried over Na₂SO₄, filtered, and concentrated under reduced pressure to afford the desired product (300 mg, 89.0% yield). LCMS (ESI) m/z: [M+H] calcd for C₁₂H₁₁FNO₃: 234.08; found 234.2.

### Intermediate 27 and 28—Synthesis of (2S,3S)-1-benzyl-3-(trifluoromethyl)aziridine-2-carboxylic Acid and (2R,3R)-1-benzyl-3-(trifluoromethyl)aziridine-2-carboxylic Acid

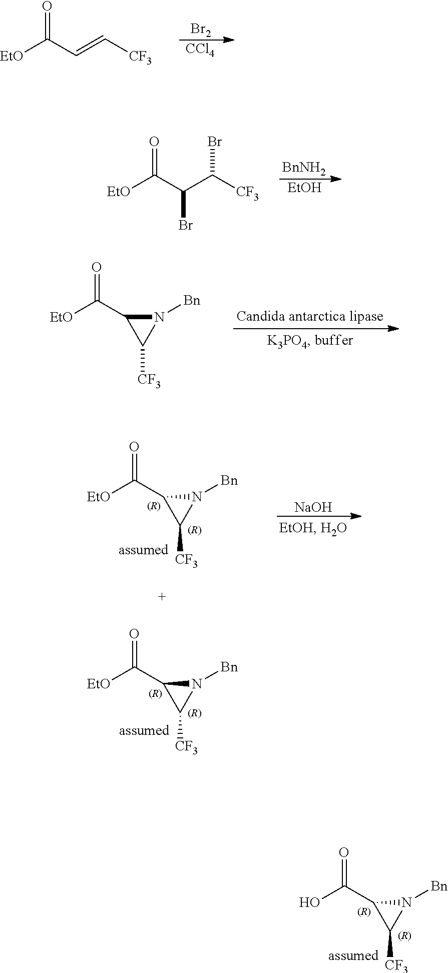

### Step 1: Synthesis of ethyl (2S,3R)-2,3-dibromo-4,4,4-trifluorobutanoate

To a solution of ethyl (E)-4,4,4-trifluorobut-2-enoate (5 g, 29.74 mmol, 4.42 mL) in CCl₄ (90 mL) was added Br₂ (1.69 mL, 32.72 mmol) and the solution was stirred at 75° C. for 5 h. The reaction mixture was concentrated under reduced pressure to give the desired product (10.72 g, crude).

### Step 2: Synthesis of ethyl (2S,3S)-1-benzyl-3-(trifluoromethyl)aziridine-2-carboxylate

To a solution of ethyl (2S,3R)-2,3-dibromo-4,4,4-trifluorobutanoate (10.72 g, 32.69 mmol) in EtOH (30 mL) was slowly added the solution of BnNH₂ (12.47 mL, 114.42 mmol) in EtOH (120 mL) at −5° C. under N₂. The mixture was warmed to room temperature and stirred for 15 h. The mixture was concentrated under reduced pressure and EtOAc (120 mL) was added to the residue. The precipitate was filtered off and the filtrate was washed with aqueous HCl (3%, 180 mL) and H₂O (100 mL), dried over Na₂SO₄, filtered and concentrated under reduced pressure. The residue was purified by silica gel chromatography (20% EtOAc/pet. ether) to afford the desired product (6.02 g, 67.4% yield).

### Step 3: Synthesis of ethyl (2R,3R)-1-benzyl-3-(trifluoromethyl)aziridine-2-carboxylate and (2S,3S)-1-benzyl-3-(trifluoromethyl)aziridine-2-carboxylic Acid

Ethyl (2R,3R)-1-benzyl-3-(trifluoromethyl)aziridine-2-carboxylate and (2S,3S)-1-benzyl-3-(trifluoromethyl)aziridine-2-carboxylic acid were synthesized in Enzyme Screening Platform, based on the procedure in Tetrahedron Asymmetry 1999, 10, 2361.

### Step 4: Synthesis of (2R,3R)-1-benzyl-3-(trifluoromethyl)aziridine-2-carboxylic Acid

To a solution of ethyl (2R,3R)-1-benzyl-3-(trifluoromethyl)aziridine-2-carboxylate (200 mg, 731.93 μmol) in EtOH (5 mL) was added NaOH (2 M, 548.95 μL) and the mixture was stirred at room temperature for 1 h. The reaction mixture was concentrated under reduced pressure to remove EtOH. Then to the mixture was added HCl (1 M) to adjust pH to 1, and extracted with EtOAc (3×5 mL). The combined organic layers were washed with brine (2×10 mL), dried over Na₂SO₄, filtered and concentrated under reduced pressure to afford the desired product (138 mg, 76.9% yield). LCMS (ESI) m/z: [M+H] calcd for C₁₁H₁₀F₃NO₂: 246.07; found 245.9.

### Intermediate 29—Synthesis of (R)-1-((benzyloxy)carbonyl)-2-methylaziridine-2-carboxylic Acid

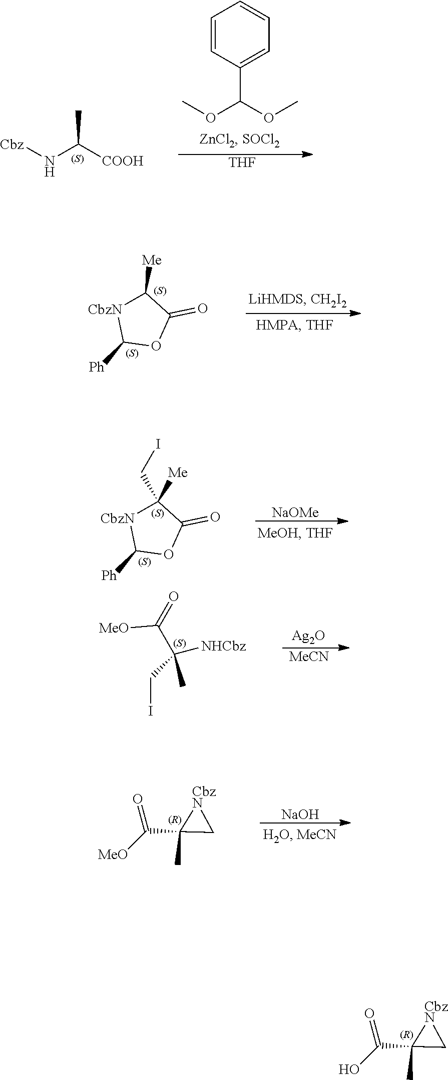

### Step 1: Synthesis of benzyl (2S,4S)-4-methyl-5-oxo-2-phenyloxazolidine-3-carboxylate

To a mixture of ((benzyloxy)carbonyl)-L-alanine (25 g, 111.99 mmol) and (dimethoxymethyl)benzene (71.38 mL, 115.35 mmol) in THF (180 mL) was added SOCl₂ (8.94 g, 123.19 mmol) in one portion at 0° C. The mixture was stirred for 10 min before ZnCl₂ (5.77 mL, 123.26 mmol) was added to the solution, then the mixture was stirred at 0° C. for 4 h. The reaction mixture was quenched by dropwise addition of cold H₂O and adjusted to pH=5 with sat. NaHCO₃, then extracted with EtOAc (2×100 mL). The organic phase was washed with a aq. sat. NaHCO₃ (30 mL) and brine (30 mL), dried over Na₂SO₄, and concentrated under reduced pressure. The residue was purified by silica gel column chromatography (1→10% EtOAc/pet. ether) to afford product (15 g, 43% yield).

### Step 2: Synthesis of benzyl (2S,4S)-4-(iodomethyl)-4-methyl-5-oxo-2-phenyloxazolidine-3-carboxylate

HMPA (5.22 mL, 29.74 mmol) and LHMDS (1 M, 6.62 mL) were mixed in THF (45 mL) under N2 atmosphere at 20° C. This solution was cooled to −78° C. and a solution of benzyl (2S,4S)-4-methyl-5-oxo-2-phenyloxazolidine-3-carboxylate (2.0 g, 6.42 mmol) in THF (12 mL) was added dropwise with stirring. After stirring an additional 30 min, a solution of CH₂I₂ (1.55 mL, 19.27 mmol) in THF (6 mL) was added dropwise. The mixture was stirred at −78° C. for 90 min. The mixture was warmed to 0° C. and quenched with sat. aq. NH₄Cl (70 mL). The mixture was extracted with EtOAc (2×30 mL), and the combined organic layers was washed with sat. aq. NH₄Cl (20 mL), H₂O (2×20 mL), and brine (30 mL) dried over Na₂SO₄, filtered, and concentrated under reduced pressure. The resulting residue was purified by silica gel column chromatography (1→20% EtOAc/pet. ether) to afford product (1.2 g, 41.4% yield).

### Step 3: Synthesis of methyl (S)-2-(((benzyloxy)carbonyl)amino)-3-iodo-2-methylpropanoate

To a mixture of benzyl (2S,4S)-4-(iodomethyl)-4-methyl-5-oxo-2-phenyloxazolidine-3-carboxylate (1.2 g, 2.66 mmol) in THF (20 mL) was added a solution of NaOMe (957.69 mg, 5.32 mmol, 30% purity) in MeOH (9 mL) dropwise over 10 min at −40° C. under N2. The mixture was stirred at −40° C. for 2 h, then warmed to −20° C. and stirred for 1 h. The reaction was quenched by addition of H₂O (20 mL), and the resulting mixture was extracted with EtOAc (3×20 mL). The combined organic layers were washed with brine (20 mL), dried over Na₂SO₄, filtered, and concentrated under reduced pressure. The residue was purified by silica gel column chromatography (1→20% EtOAc/pet. ether) to afford product (870 mg, 2.24 mmol, 84.4% yield).

### Step 4: Synthesis of 1-benzyl 2-methyl (R)-2-methylaziridine-1,2-dicarboxylate

To a mixture of methyl (S)-2-(((benzyloxy)carbonyl)amino)-3-iodo-2-methylpropanoate (0.87 g, 2.31 mmol) in MeCN (125 mL) was added Ag₂O (1.60 g, 6.92 mmol) in one portion at room temperature. The mixture was stirred at 90° C. for 30 min. The mixture was filtered and concentrated under reduced pressure to afford product (500 mg, 2.01 mmol, 86.9% yield).

### Step 5: Synthesis of 1-benzyl 2-methyl (R)-2-methylaziridine-1,2-dicarboxylate

To a mixture of 1-benzyl 2-methyl (R)-2-methylaziridine-1,2-dicarboxylate (250 mg, 1.0 mmol) in MeCN (2.5 mL) and H₂O (2.5 mL) was added NaOH (40.12 mg, 1.0 mmol) in one portion at 0° C. under N₂. The mixture was stirred at 0° C. for 30 min. The mixture was concentrated under reduced pressure to afford crude product (256 mg, crude). LCMS (ESI) m/z: [M+H] calcd for C₁₂H₁₂NO₄: 234.1; found 234.1.

### Intermediate 30—Synthesis of Potassium (S)-1-Isopropylaziridine-2-carboxylate

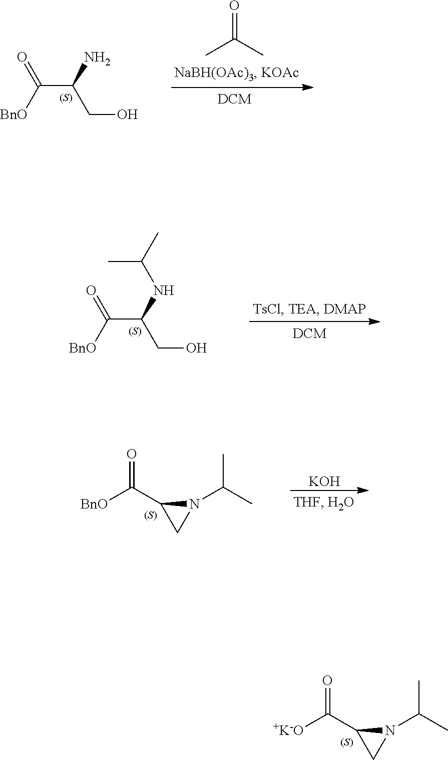

### Step 1: Synthesis benzyl isopropyl-L-serinate

To a solution of benzyl L-serinate (3.65 g, 18.69 mmol), KOAc (1.83 g, 18.69 mmol), and acetone (2.5 mL, 33.66 mmol) in DCM (60.0 mL) was added NaBH(AcO)₃ (4.76 g, 22.436 mmol) in portions at 0° C. The resulting mixture was stirred overnight at room temperature. The reaction was quenched by the addition of sat. aq. NaHCO₃ (50 mL) at room temperature. The resulting mixture was extracted with DCM (3×80 mL). The combined organic layers were washed with brine (50 mL), dried over Na₂SO₄, filtered, and concentrated under reduced pressure. The residue was purified by silica gel chromatography (67% EtOAc/hexanes) to afford the desired product (2.7 g, 60.9% yield) as an off-white solid. LCMS (ESI) m/z: [M+H] calcd for C₁₃H₁₉NO₃: 238.14; found 238.2.

### Step 2: Synthesis of benzyl (S)-1-isopropylaziridine-2-carboxylate

To a solution of benzyl isopropyl-L-serinate (2.70 g, 11.378 mmol), Et₃N (4.75 mL, 34.134 mmol) and DMAP (2.57 mg, 0.021 mmol) in DCM (50.0 mL) was added a solution of TsCl (2.60 g, 13.65 mmol) in DCM dropwise at 0° C. The resulting mixture was stirred overnight at room temperature and was then stirred for 4 h at 40° C. The reaction mixture was diluted with H₂O (80 mL) and was then extracted with DCM (2×50 mL). The combined organic layers were washed with brine (30 mL), dried over Na₂SO₄, filtered, and concentrated under reduced pressure. The residue was purified by silica gel chromatography (20% EtOAc/hexanes) to afford the desired product (2.3 g, 93.2% yield). LCMS (ESI) m/z: [M+H] calcd for C₁₃H₁₇NO₂: 220.13; found 220.1.

### Step 3: Synthesis of Potassium (S)-1-isopropylaziridine-2-carboxylate

To a solution of benzyl (S)-1-isopropylaziridine-2-carboxylate (800.0 mg, 3.65 mmol) and H₂O (6.0 mL) and THF (8.0 mL) was added a solution of KOH (245.62 mg, 4.378 mmol) in H₂O (2.0 mL) dropwise at 0° C. The resulting mixture was stirred for 2 h at room temperature. The mixture was diluted with H₂O (10 mL) and the aqueous layer was washed with MTBE (3×8 mL). The aqueous layer was dried by lyophilization to afford the desired product (400 mg, crude). LCMS (ESI) m/z: [M+H] calcd for C₆H₁₁NO₂: 130.09; found 130.0.

### Intermediate 31—Synthesis of Potassium (R)-1-Isopropylaziridine-2-carboxylate

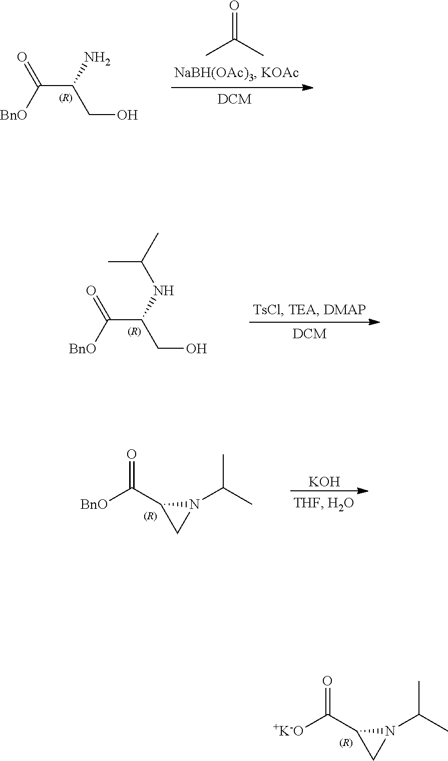

### Step 1: Synthesis benzyl isopropyl-D-serinate

To a solution of benzyl D-serinate (2.10 g, 10.757 mmol), KOAc (1.06 g, 10.757 mmol), and acetone (1.2 mL, 16.136 mmol) in DCM (40.0 mL) was added a solution of NaBH(AcO)₃ (2.96 g, 13.984 mmol) in portions at 0° C. The resulting mixture was stirred overnight at room temperature. The reaction was quenched by the addition of sat. aq. NaHCO₃ (50 mL) and the mixture was extracted with DCM (3×50 mL). The combined organic layers were washed with brine (50 mL), dried over Na₂SO₄, filtered, and concentrated under reduced pressure. The residue was purified by silica gel chromatography (67% EtOAc/hexanes) to afford the desired product (1.7 g, 66.6% yield). LCMS (ESI) m/z: [M+H] calcd for C₁₃H₁₉NO₃: 238.14; found 238.0.

### Step 2: Synthesis of benzyl (R)-1-isopropylaziridine-2-carboxylate

To a solution of benzyl isopropyl-D-serinate (1.75 g, 7.375 mmol), Et₃N (2.58 mL, 18.437 mmol) and DMAP (90.09 mg, 0.737 mmol) in DCM (30.0 mL) was added a solution of TsCl (1.69 g, 8.850 mmol) in DCM dropwise at 0° C. The resulting mixture was stirred overnight at room temperature before being stirred for 4 h at 40° C. The mixture was diluted with H₂O (80 mL) and then extracted with DCM (3×50 mL). The combined organic layers were washed with brine (50 mL), dried over Na₂SO₄, filtered, and concentrated under reduced pressure. The residue was purified by silica gel chromatography (20% EtOAc/hexanes) to afford the desired product (1.4 g, 86.6% yield). LCMS (ESI) m/z: [M+H] calcd for C₁₃H₁₇NO₂: 220.13; found 219.9.

### Step 3: Synthesis of Potassium (R)-1-isopropylaziridine-2-carboxylate

To a solution of benzyl (R)-1-isopropylaziridine-2-carboxylate (600.0 mg, 2.736 mmol) in H₂O (3.0 mL) and THF (5.0 mL) was added a solution of KOH (184.22 mg, 3.283 mmol) in H₂O (2.0 mL) dropwise at 0° C. The resulting mixture was stirred for 2 h at room temperature. The mixture was then diluted with H₂O (10 mL) and the aqueous layer was washed with MTBE (3×8 mL). The aqueous layer was then dried by lyophilization to afford the desired product (260 mg, crude). LCMS (ESI) m/z: [M+H] calcd for C₆H₁₁NO₂: 130.09; found 130.1.

### Intermediate 32—Synthesis of (S)-1-(2-((tert-butyldiphenylsilyl)oxy)ethyl)aziridine-2-carboxylic Acid

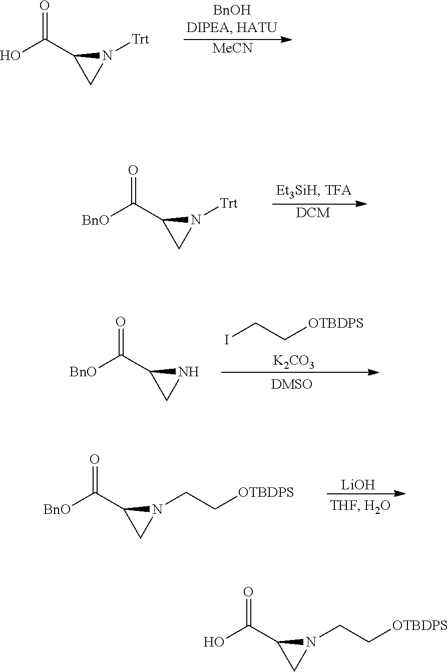

### Step 1: Synthesis of benzyl (S)-1-tritylaziridine-2-carboxylate

To a solution of (S)-1-tritylaziridine-2-carboxylic acid (500.0 mg, 1.518 mmol), benzyl alcohol (246.2 mg, 2.277 mmol) and DIPEA (0.793 mL, 4.554 mmol) in MeCN (10.0 mL) was added HATU (1.73 mg, 4.554 mmol). The resulting solution was stirred for 3 h at room temperature and was then concentrated under reduced pressure. The crude residue was purified by prep-TLC (50% EtOAc/pet. ether) to afford the desired product (300 mg, 47.1% yield) as an off-white slid. LCMS (ESI) m/z: [M+Na] calcd for C₂₉H₂₅NO₂: 442.18; found 442.3.

### Step 2: Synthesis of benzyl (S)-aziridine-2-carboxylate

To a solution of benzyl (S)-1-tritylaziridine-2-carboxylate (300.0 mg, 0.715 mmol) in DCM (5.0 mL) at 0° C. was added TFA (326.2 mg, 2.860 mmol) and Et₃SiH (332.6 mg, 2.860 mmol). The resulting mixture was stirred at 0° C. for 3 h and was then concentrated under reduced pressure. The residue was purified by prep-TLC (10% MeOH/DCM) to afford the desired product (130 mg, 82.1% yield). LCMS (ESI) m/z: [M+H] calcd for C₁₀H₁₁NO₂: 178.09; found 178.2.

### Step 3: Synthesis of benzyl (S)-1-(2-((tert-butyldiphenylsilyl)oxy)ethyl)aziridine-2-carboxylate

To a solution of benzyl (S)-aziridine-2-carboxylate (400.0 mg, 2.257 mmol) and tert-butyl(2-iodoethoxy)diphenylsilane (1.85 g, 4.52 mmol) in DMSO (10.0 mL) was added K₂CO₃ (935.9 mg, 6.772 mmol) at room temperature. The mixture was stirred at 60° C. for 5 h. The mixture was diluted with H₂O (30.0 mL) and was extracted with EtOAc (2×30 mL). The combined organic layers were washed with brine (2×50 mL), dried over Na₂SO₄, filtered, and concentrated under reduced pressure. The resulting residue was purified by prep-TLC (20% EtOAc/pet. ether) to afford the desired product (200 mg, 15.4% yield). LCMS (ESI) m/z: [M+H] calcd for C₂₈H₃₃NO₃Si: 460.23; found 460.0.

### Step 4: Synthesis of Lithium (S)-1-(2-((tert-butyldiphenylsilyl)oxy)ethyl)aziridine-2-carboxylate

To a solution of benzyl (S)-1-(2-((tert-butyldiphenylsilyl)oxy)ethyl)aziridine-2-carboxylate (200.0 mg, 0.435 mmol) in MeOH (2.0 mL) was added LiOH.H₂O (36.5 mg, 0.870 mmol). The resulting mixture was stirred overnight and was then concentrated under reduced pressure to afford the desired product (200 mg, crude). LCMS (ESI) m/z: [M+H] calcd for C₂₁H₂₇NO₃Si: 370.18; found 370.1.

### Intermediate 33—Synthesis of (R)-1-(2-((tert-butyldiphenylsilyl)oxy)ethyl)aziridine-2-carboxylic Acid

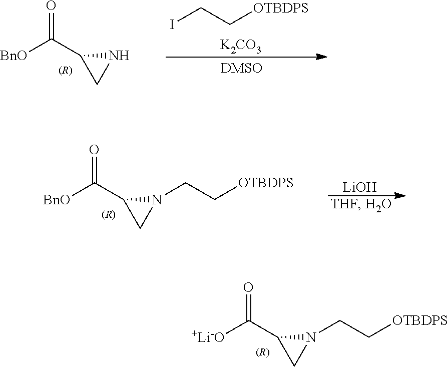

### Step 1: Synthesis of methyl benzyl (R)-1-(2-((tert-butyldiphenylsilyl)oxy)ethyl)aziridine-2-carboxylate

To a solution of benzyl (R)-aziridine-2-carboxylate (600.0 mg, 3.386 mmol) and K₂CO₃ (1.87 g, 13.544 mmol) in DMSO (8.0 mL) was added tert-butyl(2-iodoethoxy)diphenylsilane (1.39 g, 3.386 mmol) in portions at room temperature. The resulting mixture was stirred at 80° C. for 16 h. The reaction mixture was then cooled to room temperature and concentrated under reduced pressure. The residue was purified by reverse phase chromatography (60→90% MeCN/H₂O) to afford the desired product (150 mg, 9.6% yield) as a colorless solid. LCMS (ESI) m/z: [M+Na] calcd for C₂₈H₃₃NO₃Si: 482.21; found 482.3.

### Step 2: Synthesis of (R)-1-(2-((tert-butyldiphenylsilyl)oxy)ethyl)aziridine-2-carboxylic Acid

To a solution of methyl benzyl (R)-1-(2-((tert-butyldiphenylsilyl)oxy)ethyl)aziridine-2-carboxylate (180.0 mg, 0.392 mmol) in H₂O (2.0 mL) and THF (3.0 mL) at 0° C. was added a solution of LiOH.H₂O (32.87 mg, 0.392 mmol) in H₂O (1.0 mL). The resulting mixture was diluted with H₂O (6.0 mL) and the aqueous layer was washed with MTBE (3×4 mL). The aqueous layer was dried by lyophilization which afforded the desired product (140 mg, crude). LCMS (ESI) m/z: [M+H] calcd for C₂₁H+₂₇NO₃Si: 370.18; found 370.0.

### Intermediate 34—Synthesis of Lithium (S)-1-(3-methoxypropyl)aziridine-2-carboxylate

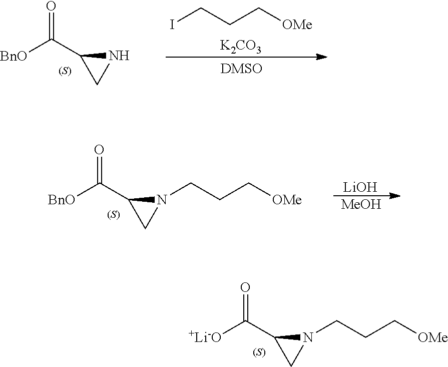

### Step 1: Synthesis of benzyl (S)-1-(3-methoxypropyl)aziridine-2-carboxylate

To a mixture of benzyl (S)-aziridine-2-carboxylate (250 mg, 1.411 mmol) and K₂CO₃ (389.96 mg, 2.822 mmol) in DMSO (4 mL) at 60° C. was added 1-iodo-3-methoxypropane (564.38 mg, 2.822 mmol). The resulting mixture was stirred for 2 h and was then cooled to room temperature, diluted with brine (50 mL), and extracted with EtOAc (3×20 mL). The combined organic layers were concentrated under reduced pressure. The crude product was purified by reverse phase chromatography (25%→40% H₂O/MeCN) to afford the desired product (234 mg, 63.2% yield). LCMS (ESI) m/z: [M+H] calcd for C₁₄H₁₉NO₃: 250.14; found 250.2.

### Step 2: Synthesis of Lithium (S)-1-(3-methoxypropyl)aziridine-2-carboxylate

A mixture of benzyl (S)-1-(3-methoxypropyl) aziridine-2-carboxylate (230 mg, 0.923 mmol) and LiOH.H₂O (77.43 mg, 1.845 mmol) in MeOH (3 mL) was stirred for 1 h at 0° C. The resulting mixture was concentrated under reduced pressure to afford the desired product (320 mg, crude). LCMS (ESI) m/z: [M+H] calcd for C₇H₁₃NO₃: 160.09; found 160.1.

### Intermediate 35—Synthesis of Lithium (R)-1-(3-methoxypropyl)aziridine-2-carboxylate

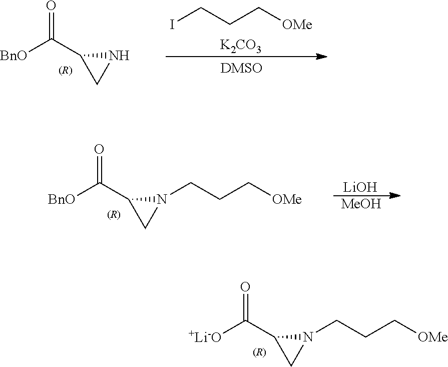

### Step 1: Synthesis of benzyl (R)-1-(3-methoxypropyl)aziridine-2-carboxylate

To a mixture of benzyl (R)-aziridine-2-carboxylate (350.0 mg, 1.975 mmol) and K₂CO₃ (545.95 mg, 3.950 mmol) in DMSO (4 mL) at 60° C. was added 1-iodo-3-methoxypropane (790.13 mg, 3.950 mmol). The resulting mixture was stirred for 2 h and was then cooled to room temperature, diluted with brine (50 mL), and extracted with EtOAc (3×20 mL). The combined organic layers were concentrated under reduced pressure. The crude product was purified by reverse phase chromatography (30%→38% MeCN/H₂O) to afford the desired product (170 mg, 31.1% yield). LCMS (ESI) m/z: [M+H] calcd for C₁₄H₁₉NO₃: 250.14; found 250.2.

### Step 2: Synthesis of Lithium (R)-1-(3-methoxypropyl)aziridine-2-carboxylate

A mixture of benzyl (R)-1-(3-methoxypropyl)aziridine-2-carboxylate (170 mg, 0.682 mmol) and LiOH (57.23 mg, 1.364 mmol) in MeOH (2 mL) was stirred at 0° C. for 1 h. The mixture was concentrated under reduced pressure to afford the desired product (200 mg, crude). LCMS (ESI) m/z: [M+H] calcd for C₇H₁₃NO₃: 160.09; found 160.3.

### Intermediate 36—Synthesis of (S)-1-((3-methyloxetan-3-yl)methyl)aziridine-2-carboxylic Acid

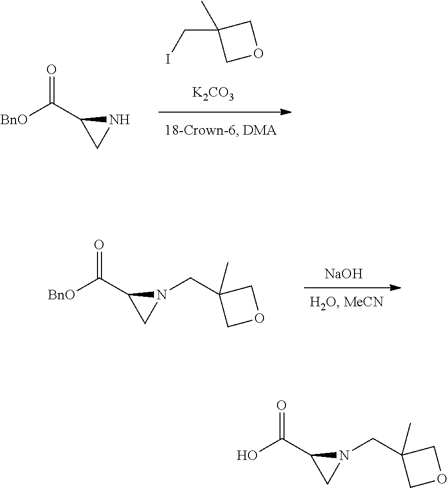

### Step 1: Synthesis of benzyl (S)-1-((3-methyloxetan-3-yl)methyl)aziridine-2-carboxylate

To a mixture of benzyl (S)-aziridine-2-carboxylate (440 mg, 2.48 mmol) and 3-(iodomethyl)-3-methyloxetane (2.11 g, 9.93 mmol) in DMA (5 mL) was added K₂CO₃ (1.72 g, 12.42 mmol) and 18-crown-6 (32.8 mg, 124 μmol). The reaction mixture was heated to 80° C. and stirred for 12 h, and was then was diluted with H₂O (25 mL) and EtOAc (25 mL). The aqueous layer was extracted with EtOAc (3×20 mL), and the combined organic layers were washed with brine (5×45 mL), dried over Na₂SO₄, filtered, and concentrated under reduced pressure. Purification by prep-TLC (50% EtOAc/pet. ether) afforded the desired product (367 mg, 57% yield). LCMS (ESI) m/z: [M+H] calcd for C₁₅H₁₉NO₃: 262.14; found 262.0.

### Step 2: Synthesis of (S)-1-((3-methyloxetan-3-yl)methyl)aziridine-2-carboxylic Acid

To a mixture of benzyl (S)-1-((3-methyloxetan-3-yl)methyl)aziridine-2-carboxylate (100 mg, 383 μmol) in MeCN (500 μL) and H₂O (500 μL) at 0° C. was added NaOH (23 mg, 574 μmol). The reaction mixture was stirred at 0° C. for 1 h then was concentrated under reduced pressure to afford the desired product (100 mg, crude). LCMS (ESI) m/z: [M+H] calcd for C₈H₁₃NO₃: 172.10; found 172.0.

### Intermediate 37—Synthesis of (2R,3R)-3-methyloxirane-2-carboxylic Acid

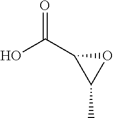

This intermediate was synthesized following the procedures outlined in WO2014071565 A1, which is incorporated by reference in its entirety.

### Intermediate 38—Synthesis of (2S,3R)-3-methyloxirane-2-carboxylic Acid

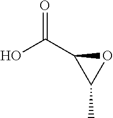

This intermediate was synthesized following the procedures outlined in Chem. Pharm. Bull. 1990, 38, 323-328, which is incorporated by reference in its entirety.

### Intermediate 39—Synthesis of (2R,3R)-3-phenyloxirane-2-carboxylic Acid

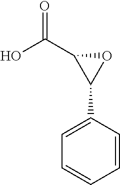 [SMILES: O=C(O)[C@@H]1O[C@@H]1c1ccccc1]

This intermediate was synthesized following the procedures outlined in J. Org. Chem. 1986, 51, 48-50, which is incorporated by reference in its entirety.

### Intermediate 40—Synthesis of (2R,3S)-3-phenyloxirane-2-carboxylic Acid

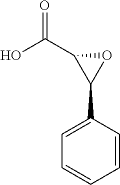 [SMILES: O=C(O)[C@@H]1O[C@H]1c1ccccc1]

This intermediate was synthesized following the procedures outlined in J. Org. Chem. 1993, 58, 7615-7618, which is incorporated by reference in its entirety.

### Intermediate 41—Synthesis of (2R,3R)-3-vinyloxirane-2-carboxylic Acid

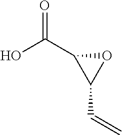

This intermediate was synthesized following the procedures outlined in Helv. Chim. Acta. 2013, 96, 268-274, which is incorporated by reference in its entirety.

### Example 1—Synthesis of N-benzyl-N-(1-(6-chloro-8-fluoro-7-(2-fluorophenyl)quinazolin-4-yl)piperidin-4-yl)methanediimine

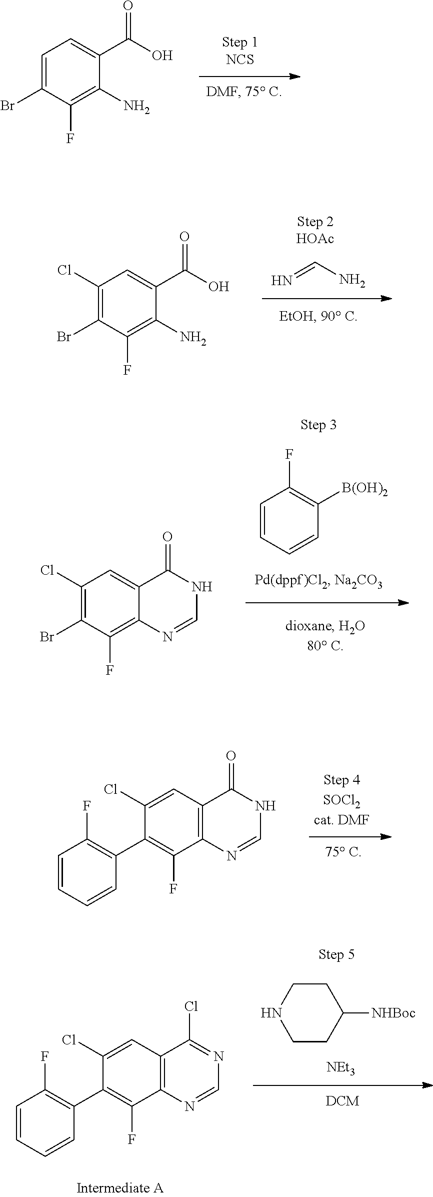

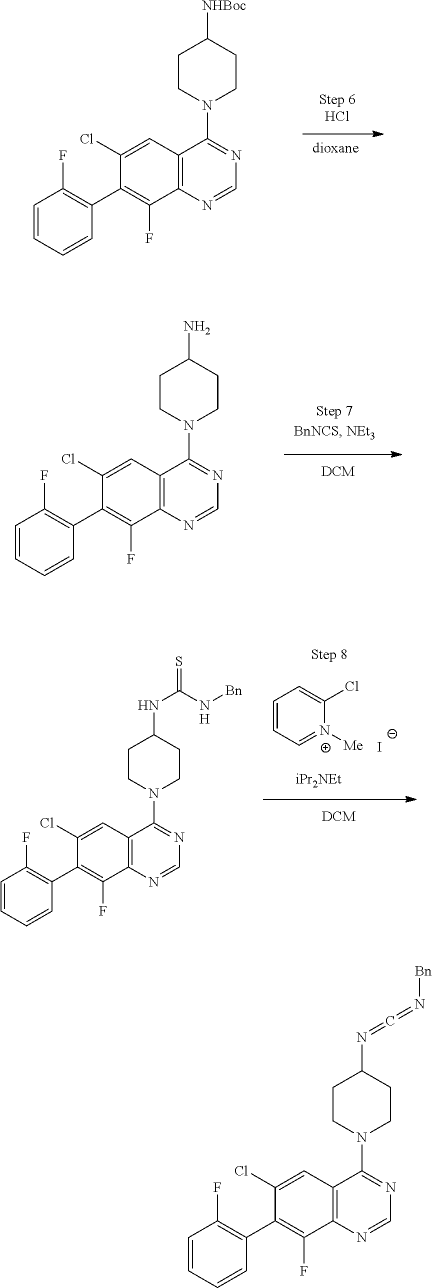

### Step 1: Synthesis of 2-amino-4-bromo-5-chloro-3-fluorobenzoic Acid

Three separate reactions were run in parallel. For each reaction, a mixture of 2-amino-4-bromo-3-fluorobenzoic acid (36 g, 154 mmol, 1 equiv) and NCS) (20.54 g, 154 mmol, 1 equiv) in DMF (2 L) was heated to 75° C. for 16 h then cooled to room temperature. The three separate reaction mixtures were combined and poured into ice cold H₂O (9 L). The resulting solids were isolated by vacuum filtration and dried under reduced pressure to afford 2-amino-4-bromo-5-chloro-3-fluorobenzoic acid (108 g, 87% yield) as a yellow solid. LCMS (ESI) m/z: [M+H] calcd for C₇H₅BrClFNO₂: 267.92; found 270.1. ¹H NMR (400 MHz, DMSO-d₆) δ 7.67 (d, J=1.83 Hz, 1H).

### Step 2: Synthesis of 7-bromo-6-chloro-8-fluoroquinazolin-4(3H)-one

Three separate reactions were run in parallel. For each reaction, to a solution of 2-amino-4-bromo-5-chloro-3-fluoro-benzoic acid (35 g, 130 mmol, 1 equiv) in EtOH (800 mL) was added formamidine acetate (149 g, 1.43 mol, 11 equiv). The reaction mixture was heated to 90° C. for 16 h then cooled to room temperature. The three separate reaction mixtures were combined and concentrated under reduced pressure. The residue was washed with H₂O (2×1 L), EtOAc (2×100 mL), then the solids were isolated by vacuum filtration and dried under reduced pressure to afford 7-bromo-6-chloro-8-fluoro-3H-quinazolin-4-one (80 g, 69% yield) as a yellow solid. LCMS (ESI) m/z: [M+H] calcd for C₆H₄BrClFN₂O: 276.92; found 276.9. ¹H NMR (400 MHz, DMSO-d₆) δ 12.69 (br s, 1H) 8.21 (s, 1H) 8.05 (d, J=1.34 Hz, 1H).

### Step 3: Synthesis of 6-chloro-8-fluoro-7-(2-fluorophenyl)quinazolin-4(3H)-one

To a mixture of 7-bromo-6-chloro-8-fluoro-3H-quinazolin-4-one (35.0 g, 126 mmol, 1 equiv) and 2-fluorophenylboronic acid (44.1 g, 315 mmol, 2.5 equiv) in dioxane (1.2 L) and H₂O (350 mL) was added portion-wise Pd(dppf)Cl₂ (9.23 g, 12.6 mmol, 0.10 equiv) and Na₂CO₃ (40.11 g, 378 mmol, 3 equiv). The resulting mixture was heated to 85° C. After 16 h the reaction was cooled to room temperature. The resulting mixture was filtered, and the filter cake was washed with EtOAc (3×30 mL). The filtrate was concentrated under reduced pressure. The residue was partially purified by silica gel column chromatography (1% petroleum ether/EtOAc), to afford crude product (16 g) as a yellow solid. The crude product was further purified by reverse phase chromatography (10→50% MeCN/H₂O, 0.05% NH₄HCO₃) to afford 6-chloro-8-fluoro-7-(2-fluorophenyl)-3H-quinazolin-4-one (9.5 g, 26% yield) an off-white solid. LCMS (ESI) m/z: [M+H] calcd for C₁₄H₈ClF₂N₂O: 293.03; found 293.2. ¹H NMR (400 MHz, DMSO-d₆) δ 12.68 (s, 1H), 8.24 (s, 1H), 8.08 (d, J=2.4 Hz, 1H), 7.93-7.65 (m, 1H), 7.64-7.38 (m, 3H).

### Step 4: Synthesis of 4,6-dichloro-8-fluoro-7-(2-fluorophenyl)quinazoline

To a solution of 6-chloro-8-fluoro-7-(2-fluorophenyl)quinazolin-4(3H)-one (5.1 g, 17.4 mmol, 1 equiv) in thionyl chloride (40 mL, 548 mmol, 31.5 equiv) was added DMF (0.1 mL). The resulting mixture was heated to 75° C. After 17 h, the reaction was cooled to room temperature and concentrated under reduced pressure. The crude residue was purified by silica gel column chromatography (0→100% EtOAc/hexanes, 1.0% NEt₃) to afford 4,6-dichloro-8-fluoro-7-(2-fluorophenyl)quinazoline (4.7 g, 87% yield) as a white solid. LCMS (ESI) m/z: [M+H] calcd for C₁₄H₁₇C_{I2}F₂N₂: 311.00; found 311.3.

### Step 5: Synthesis of tert-butyl (1-(6-chloro-8-fluoro-7-(2-fluorophenyl)quinazolin-4-yl)piperidin-4-yl)carbamate

To a solution of 4,6-dichloro-8-fluoro-7-(2-fluorophenyl)quinazoline (500 mg, 1.6 mmol, 1 equiv) in DCM (16 mL) was added NEt₃ (444 μL, 3.20 mmol, 2 equiv) followed by tert-butyl piperidin-4-ylcarbamate (480 mg, 2.4 mmol, 1.5 equiv). The resulting mixture was stirred for 20 min then diluted with DCM (100 mL), washed with sat. aq. NH₄Cl (50 mL) then sat. aq. NaCl (50 mL), dried over Na₂SO₄, filtered, and concentrated under reduced pressure. The resulting crude product was taken on without further purification. LCMS (ESI) m/z: [M+H] calcd for C₂₄H₂₆ClF₂N₄O₂: 475.17; found 475.4.

### Step 6: Synthesis of 1-(6-chloro-8-fluoro-7-(2-fluorophenyl)quinazolin-4-yl)piperidin-4-amine Hydrochloride

To a suspension of tert-butyl N-{1-[6-chloro-8-fluoro-7-(2-fluorophenyl)quinazolin-4-yl]piperidin-4-yl}carbamate (911 mg, 1.92 mmol) in dioxane (4.8 mL) was added slowly HCl (4 M in dioxane, 4.80 mL, 19.2 mmol, 10 equiv). The resulting mixture was stirred for 5 h then concentrated under reduced pressure. The crude product was concentrated from DCM (2×5 mL) under reduced pressure and taken on without further purification. LCMS (ESI) m/z: [M+H] calcd for C₁₉H₁₈ClF₂N₄: 375.12; found 375.3.

### Step 7: Synthesis of 1-benzyl-3-(1-(6-chloro-8-fluoro-7-(2-fluorophenyl)quinazolin-4-yl)piperidin-4-yl)thiourea

To a solution of 1-[6-chloro-8-fluoro-7-(2-fluorophenyl)quinazolin-4-yl]piperidin-4-amine hydrochloride (100 mg, 266 μmol, 1 equiv) in DCM (2.7 mL) was added NEt₃ (111 μL, 798 μmol, 3 equiv) followed by benzyl isothiocyanate (35.2 μL, 266 μmol, 1 equiv). The resulting mixture was stirred for 20 h then diluted with DCM (40 mL), washed with H₂O (20 mL) followed by sat. aq. NaCl (20 mL), dried over Na₂SO₄, filtered, and concentrated under reduced pressure. The resulting crude product was used without further purification. LCMS (ESI) m/z: [M+H] calcd for C₂₇H₂₅ClF₂N₅S: 524.15; found 524.6.

### Step 8: Synthesis of N-benzyl-N-(1-(6-chloro-8-fluoro-7-(2-fluorophenyl)quinazolin-4-yl)piperidin-4-yl)methanediimine

To a solution of 1-benzyl-3-{1-[6-chloro-8-fluoro-7-(2-fluorophenyl)quinazolin-4-yl]piperidin-4-yl}thiourea (64 mg, 122 μmol, 1 equiv) in DCM (1.22 mL) was added N,N-diisopropylethylamine (63.7 μL, 366 μmol, 3 equiv) followed by 2-chloro-1-methylpyridin-1-ium iodide (46.7 mg, 183 μmol, 1.5 equiv). The resulting mixture was stirred for 18 h then filtered to remove solids. The filtrate was diluted with DCM (20 mL), washed with H₂O (10 mL) then sat. aq. NaCl (10 mL), dried over Na₂SO₄, filtered, and concentrated under reduced pressure. The crude product was purified by reverse phase chromatography (60→100% MeCN/H₂O) to afford N-benzyl-N-(1-(6-chloro-8-fluoro-7-(2-fluorophenyl)quinazolin-4-yl)piperidin-4-yl)methanediimine (11.9 mg, 20% yield over 4 steps) as a white solid. LCMS (ESI) m/z: [M+H] calcd for C₂₇H₂₃ClF₂N₅: 490.16; found 490.2. ¹H NMR (500 MHz, Methanol-d₄) δ 8.63 (s, 1H), 7.92 (d, J=1.8 Hz, 1H), 7.63-7.56 (m, 1H), 7.46 (td, J=7.5, 1.9 Hz, 1H), 7.41-7.35 (m, 5H), 7.35-7.26 (m, 2H), 4.36 (s, 2H), 4.16-4.04 (m, 2H), 3.72-3.49 (m, 3H), 2.04-1.87 (m, 2H), 1.59-1.46 (m, 2H).

### Example 2—Synthesis of N-(1-(6-chloro-8-fluoro-7-(2-fluorophenyl)quinazolin-4-yl)piperidin-4-yl)-N-phenylmethanediimine

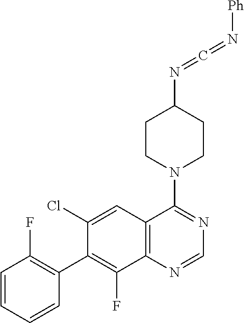 [SMILES: Fc1ccccc1-c1c(Cl)cc2c(N3CCC(N=C=Nc4ccccc4)CC3)ncnc2c1F]

Synthesized according to the method of example 1, using phenyl isothiocyanate in place of benzyl isothiocyanate in step 7. LCMS (ESI) m/z: [M+H] calcd for C₂₆H₂₁ClF₂N₅: 476.15; found 476.1.

### Example 3—Synthesis of N-(1-(6-chloro-1-fluoro-7-(2-fluorophenyl)quinazolin-4-yl)piperidin-4-yl)-N-isopropylmethanediimine

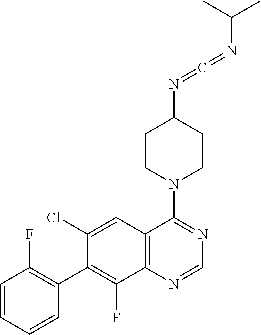 [SMILES: CC(C)N=C=NC1CCN(c2ncnc3c(F)c(-c4ccccc4F)c(Cl)cc23)CC1]

Synthesized according to the method of example 1, using 2-propyl isothiocyanate in place of benzyl isothiocyanate in step 7. LCMS (ESI) m/z: [M+H] calcd for C₂₃H₂₃ClF₂N₅: 442.16; found 442.1.

### Example 4—Synthesis of N-(1-(6-chloro-8-fluoro-7-(2-fluorophenyl)quinazolin-4-yl)piperidin-4-yl)-N-methylmethanediimine

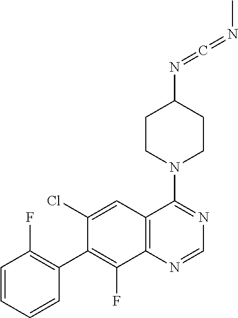 [SMILES: CN=C=NC1CCN(c2ncnc3c(F)c(-c4ccccc4F)c(Cl)cc23)CC1]

Synthesized according to the method of example 1, using methyl isothiocyanate in place of benzyl isothiocyanate in step 7. LCMS (ESI) m/z: [M+H] calcd for C₂₁H₁₉ClF₂N₅: 414.13, found 414.1.

### Example 5—Synthesis of N-(1-(6-chloro-8-fluoro-7-(2-fluorophenyl)quinazolin-4-yl)piperidin-4-yl)-N-(4-methoxyphenyl)methanediimine

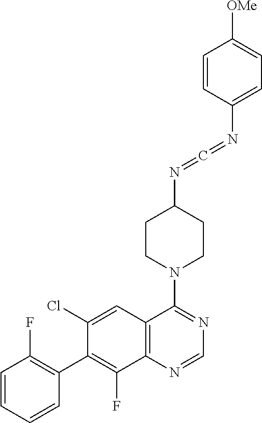 [SMILES: COc1ccc(N=C=NC2CCN(c3ncnc4c(F)c(-c5ccccc5F)c(Cl)cc34)CC2)cc1]

Synthesized according to the method of example 1, using 4-methoxyphenyl isothiocyanate in place of benzyl isothiocyanate in step 7. LCMS (ESI) m/z: [M+H] calcd for C₂₇H₂₃ClF₂N₅O: 506.16; found 506.2.

### Example 6—Synthesis of N-(1-(6-chloro-8-fluoro-7-(2-fluorophenyl)quinazolin-4-yl)piperidin-4-yl)-N-(4-chiorophenyl)methanediimine

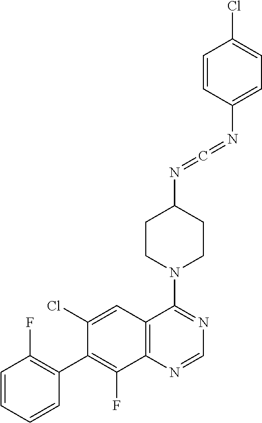 [SMILES: Fc1ccccc1-c1c(Cl)cc2c(N3CCC(N=C=Nc4ccc(Cl)cc4)CC3)ncnc2c1F]

Synthesized according to the method of example 1, using 4-chlorophenyl isothiocyanate in place of benzyl isothiocyanate in step 7. LCMS (ESI) m/z: [M+H] calcd for C₂₆H₂₀Cl₂F₂N₅: 510.11; found 510.1.

### Example 7—Synthesis of 6-chloro-8-fluoro-7-(2-fluorophenyl)-N-methyl-N-(2-(((phenylimino)methylene)amino)ethyl)quinazolin-4-amine

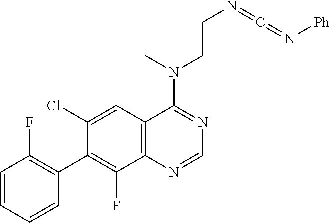 [SMILES: CN(CCN=C=Nc1ccccc1)c1ncnc2c(F)c(-c3ccccc3F)c(Cl)cc12]

Synthesized according to the method of example 1, using tert-butyl (2-(methylamino)ethyl)carbamate in place of by tert-butyl piperidin-4-ylcarbamate in step 5 and phenyl isothiocyanate in place of benzyl isothiocyanate in step 7. LCMS (ESI) m/z: [M+H] calcd for C₂₄H₁₉ClF₂N₅: 450.90; found 450.1.

### Example 8—Synthesis of N-(2-(((benzylimino)methylene)amino)ethyl)-6-chloro-8-fluoro-7-(2-fluorophenyl)-N-methylquinazolin-4-amine

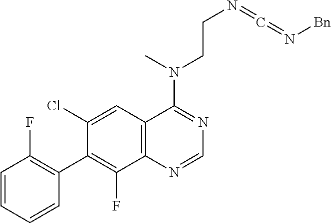 [SMILES: CN(CCN=C=NCc1ccccc1)c1ncnc2c(F)c(-c3ccccc3F)c(Cl)cc12]

Synthesized according to the method of example 1, using tert-butyl (2-(methylamino)ethyl)carbamate in place of by tert-butyl piperidin-4-ylcarbamate in step 5. LCMS (ESI) m/z: [M+H] calcd for C₂₅H₂₁ClF₂N₅: 484.92; found 484.2.

### Example 9—Synthesis of 6-chloro-8-fluoro-7-(2-fluorophenyl)-N-methyl-N-(2-(((methylimino)methylene)amino)ethyl)quinazolin-4-amine

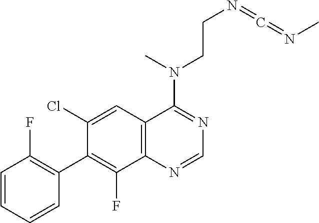 [SMILES: CN=C=NCCN(C)c1ncnc2c(F)c(-c3ccccc3F)c(Cl)cc12]

Synthesized according to the method of example 1, using tert-butyl (2-(methylamino)ethyl)carbamate in place of by tert-butyl piperidin-4-ylcarbamate in step 5 and methyl isothiocyanate in place of benzyl isothiocyanate in step 7. LCMS (ESI) m/z: [M+H] calcd for C₁₉H₁₇ClF₂N₅: 388.83; found 388.1.

### Example 10—Synthesis of 1-(1-(6-chloro-8-fluoro-7-(2-fluorophenyl)quinazolin-4-yl)piperidin-4-yl)-3-(2-chloroethyl)urea

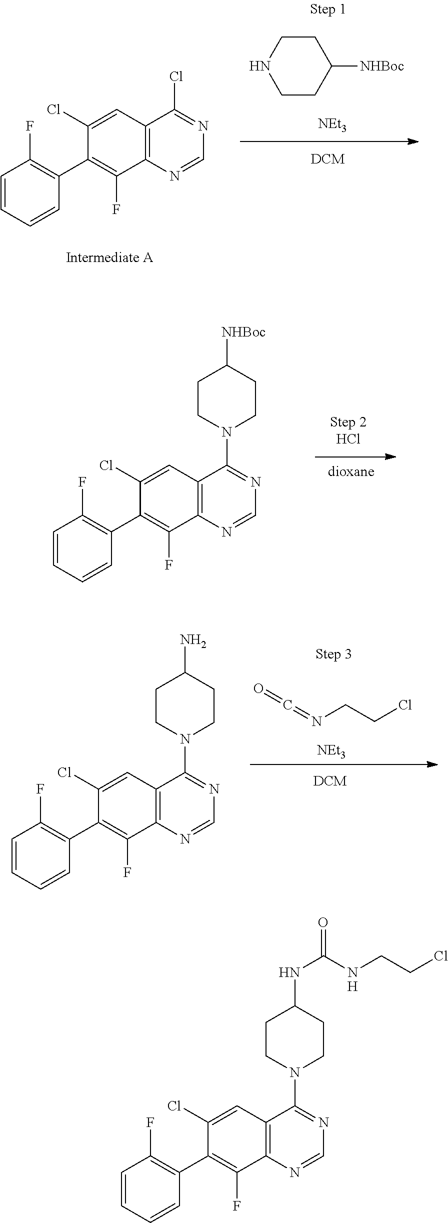

### Step 1: Synthesis of tert-butyl (1-(6-chloro-8-fluoro-7-(2-fluorophenylquinazolin-4-yl)piperidin-4-yl)carbamate

To a solution of 4,6-dichloro-8-fluoro-7-(2-fluorophenyl)quinazoline (500 mg, 1.6 mmol, 1 equiv) in DCM (16 mL) was added NEt₃ (444 μL, 3.20 mmol, 2 equiv) followed by tert-butyl piperidin-4-ylcarbamate (480 mg, 2.4 mmol, 1.5 equiv). The resulting mixture was stirred for 20 min then diluted with DCM (100 mL), washed with sat. aq. NH₄Cl (50 mL) then sat. aq. NaCl (50 mL), dried over Na₂SO₄, filtered, and concentrated under reduced pressure. The resulting crude product was taken on without further purification. LCMS (ESI) m/z: [M+H] calcd for C₂₄H₂₆ClF₂N₄O₂: 475.17; found 475.4.

### Step 2: Synthesis of 1-(6-chloro-8-fluoro-7-(2-fluorophenyl)quinazolin-4-yl)piperidin-4-amine

To a suspension of tert-butyl N-{1-[6-chloro-8-fluoro-7-(2-fluorophenyl)quinazolin-4-yl]piperidin-4-yl}carbamate (911 mg, 1.92 mmol) in dioxane (4.8 mL) was slowly added HCl (4 M in dioxane, 4.80 mL, 19.2 mmol, 10 equiv). The resulting mixture was stirred for 5 h then concentrated under reduced pressure. The crude product was concentrated from DCM (2×5 mL) under reduced pressure and taken on without further purification. LCMS (ESI) m/z: [M+H] calcd for C₁₉H₁₈ClF₂N₄: 375.12; found 375.3.

### Step 3: Synthesis of 1-(1-(6-chloro-8-fluoro-7-(2-fluorophenyl)quinazolin-4-yl)piperidin-4-yl)-3-(2-chloroethyl)urea

To a suspension of 1-[6-chloro-8-fluoro-7-(2-fluorophenylquinazolin-4-yl]piperidin-4-amine (145 mg, 386 μmol, 1 equiv) in DCM (1.5 mL) was added NEt₃ (159 μL, 1.15 mmol, 3.0 equiv) followed by 1-chloro-2-isocyanatoethane (32.9 μL, 386 μmol, 1 equiv). The resulting mixture was stirred for 30 min and then concentrated under reduced pressure. The residue was purified by silica gel column chromatography (2→25% MeOH/DCM, 1.0% NEt₃) to afford 1-(1-(6-chloro-8-fluoro-7-(2-fluorophenyl)quinazolin-4-yl)piperidin-4-yl)-3-(2-chloroethyl)urea (57.6 mg, 31% yield) as a white solid. LCMS (ESI) m/z: [M+H] calcd for C₂₂H₂₂Cl₂F₂N₅O: 480.12; found 480.5. ¹H NMR (500 MHz, Chloroform-d) δ 8.80 (s, 1H), 7.82 (d, J=1.5 Hz, 1H), 7.57-7.48 (m, 1H), 7.39 (td, J=7.4, 1.8 Hz, 1H), 7.32 (td, J=7.5, 1.0 Hz, 1H), 7.28-7.23 (m, 1H), 4.86-4.77 (m, 1H), 4.55-4.47 (m, 1H), 4.41 (t, J=11.9 Hz, 2H), 4.09-3.97 (m, 1H), 3.67 (t, J=5.3 Hz, 2H), 3.59 (q, J=5.4 Hz, 2H), 3.50-3.35 (m, 2H), 2.20 (d, J=13.2 Hz, 2H), 1.72-1.58 (m, 2H).

### Example 11—Synthesis of 4-(6-chloro-8-fluoro-7-(2-fluorophenyl)quinazolin-4-yl)-N-(2-chloroethyl)piperazine-1-carboxamide

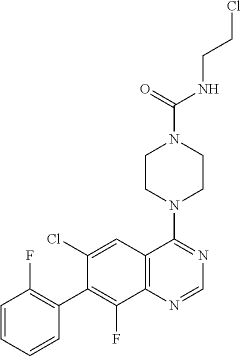 [SMILES: O=C(NCCCl)N1CCN(c2ncnc3c(F)c(-c4ccccc4F)c(Cl)cc23)CC1]

Synthesized according to the method of example 10, using tert-butyl piperazine-1-carboxylate in place of tert-butyl piperidin-4-ylcarbamate in step 1. LCMS (ESI) m/z: [M+H] calcd for C₂₁H₂₀Cl₂F₂N₅O: 466.10; found 466.3.

### Example 12—Synthesis of 1-(2-((6-chloro-8-fluoro-7-(2-fluorophenyl)quinazolin-4-yl)amino)ethyl)-3-(2-chloroethyl)urea

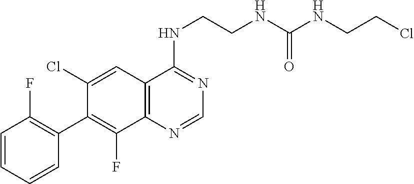 [SMILES: O=C(NCCCl)NCCNc1ncnc2c(F)c(-c3ccccc3F)c(Cl)cc12]

Synthesized according to the method of example 10, using tert-butyl (2-aminoethyl)carbamate in place of tert-butyl piperidin-4-ylcarbamate in step 1. LCMS (ESI) m/z: [M+H] calcd for C₁₉H₁₈Cl₂F₂N₅O: 440.09; found 440.1.

### Example 13—Synthesis of N-(1-(6-chloro-8-fluoro-7-(2-fluorophenyl)quinazolin-4-yl)piperidin-4-yl)-4,5-dihydrooxazol-2-amine

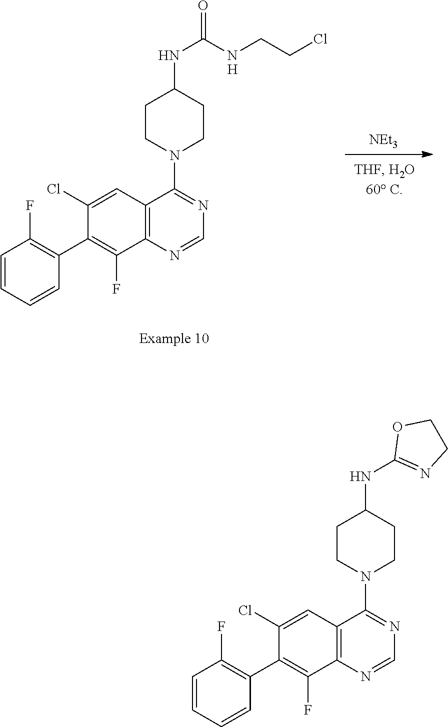

To a suspension of 1-{1-[6-chloro-8-fluoro-7-(2-fluorophenyl)quinazolin-4-yl]piperidin-4-yl}-3-(2-chloroethyl)urea (44 mg, 91.6 μmol, 1 equiv) in THF (915 μL) and H₂O (915 μL) was added NEt₃ (15.1 μL, 109 μmol, 1.2 equiv). The resulting mixture was heated to 60° C. After 18 h the reaction was cooled to room temperature and concentrated under reduced pressure. The crude residue was purified by reverse phase chromatography (10→99% MeCN/H₂O, 0.1% NEt₃) to afford N-(1-(6-chloro-8-fluoro-7-(2-fluorophenyl)quinazolin-4-yl)piperidin-4-yl)-4,5-dihydrooxazol-2-amine (17.2 mg, 42%). LCMS (ESI) m/z: [M+H] calcd for C₂₂H₂₁ClF₂N₅O: 444.14; found 444.2. ¹H NMR (500 MHz, Chloroform-d) δ 8.80 (s, 1H), 7.81 (d, J=1.7 Hz, 1H), 7.55-7.48 (m, 1H), 7.39 (td, J=7.4, 1.8 Hz, 1H), 7.32 (td, J=7.5, 1.1 Hz, 1H), 7.28-7.24 (m, 1H), 4.35 (t, J=8.6 Hz, 4H), 3.85 (t, J=8.5 Hz, 3H), 3.39 (qd, J=13.7, 2.6 Hz, 2H), 2.28 (dd, J=12.7, 3.0 Hz, 2H), 1.77-1.64 (m, 2H).

### Example 14—Synthesis of 2-(4-(6-chloro-8-fluoro-7-(2-fluorophenyl)quinazolin-4-yl)piperazin-1-yl)-4,5-dihydrooxazole

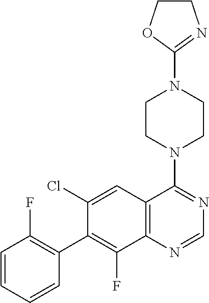 [SMILES: Fc1ccccc1-c1c(Cl)cc2c(N3CCN(C4=NCCO4)CC3)ncnc2c1F]

Synthesized according to the method of example 13, using example 11 in place of example 10. LCMS (ESI) m/z: [M+H] calcd for C₂₁H₁₉ClF₂N₅O: 430.13; found 430.4.

### Example 15—Synthesis of N-(6-chloro-8-fluoro-7-(2-fluorophenyl)quinazolin-4-yl)-N²-(4,5-dihydrooxazol-2-yl)ethane-1,2-diamine

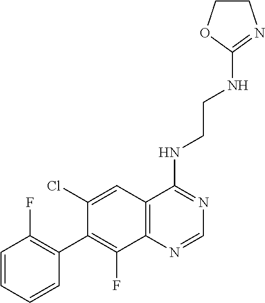 [SMILES: Fc1ccccc1-c1c(Cl)cc2c(NCCNC3=NCCO3)ncnc2c1F]

Synthesized according to the method of example 13, using example 12 in place of example 10. LCMS (ESI) m/z: [M+H] calcd for C₁₉H₁₇ClF₂N₅O: 404.11; found 404.4.

### Example 16—Synthesis of aziridin-2-yl(4-(6-chloro-8-fluoro-7-(2-fluorophenyl)quinazolin-4-yl)piperazin-1-yl)methanone

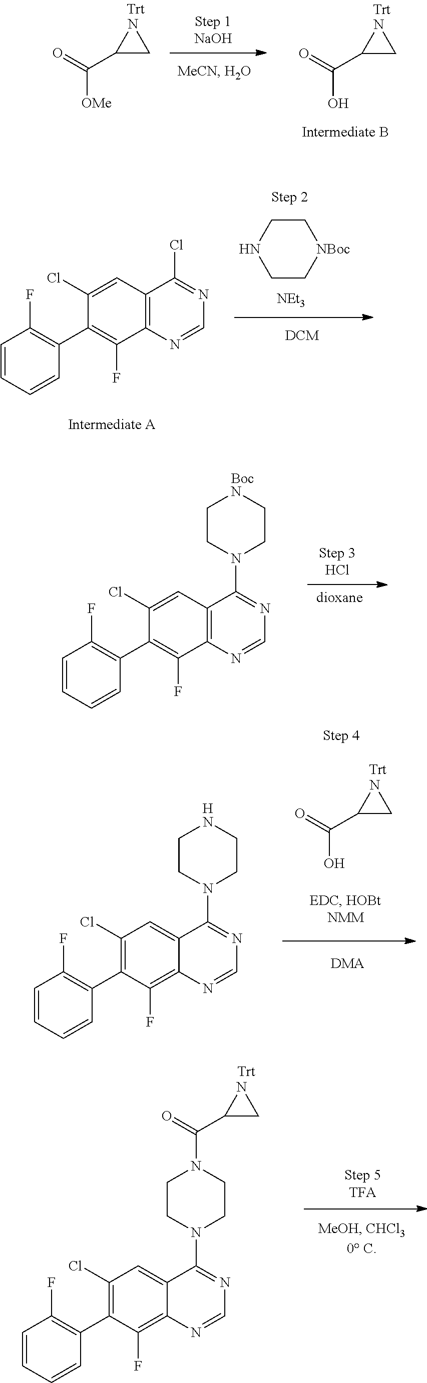

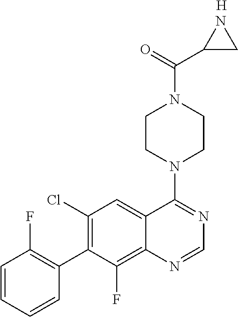

### Step 1: Synthesis of 1-tritylaziridine-2-carboxylic Acid

To a solution of methyl 1-tritylaziridine-2-carboxylate (300 mg, 0.873 mmol, 1 equiv) in MeCN (1.57 mL) was added a solution of sodium hydroxide (52.4 mg, 1.31 mmol, 1.5 equiv) in H₂O (1.57 mL). The resulting mixture was stirred for 18 h then concentrated under reduced pressure to afford 1-tritylaziridine-2-carboxylic acid that was used without further purification.

### Step 2: Synthesis of tert-butyl 4-(6-chloro-8-fluoro-7-(2-fluorophenyl)quinazolin-4-yl)piperazine-1-carboxylate

To a suspension of 4,6-dichloro-8-fluoro-7-(2-fluorophenyl)quinazoline (203 mg, 520 μmol, 1 equiv) and powdered 3 Λ mol. sieves (200 mg) in DCM (13 mL) was added NEt₃ (143 μL, 1.03 mmol, 2 equiv) followed by tert-butyl piperazine-1-carboxylate (129 mg, 692 μmol, 1.3 equiv). The resulting mixture was stirred for 1 h then concentrated under reduced pressure. The residue was purified by silica gel column chromatography (0→100% EtOAc/hexanes) to afford tert-butyl 4-(6-chloro-8-fluoro-7-(2-fluorophenyl)quinazolin-4-yl)piperazine-1-carboxylate. LCMS (ESI) m/z: [M+H] calcd for C₂₃H₂₄ClF₂N₄O₂: 461.16; found 461.4.

### Step 3: Synthesis of 6-chloro-8-fluoro-7-(2-fluorophenyl)-4-(piperazin-1-yl)quinazoline

A suspension of tert-butyl 4-(6-chloro-8-fluoro-7-(2-fluorophenyl)quinazolin-4-yl)piperazine-1-carboxylate (90 mg, 195 μmol, 1 equiv) in HCl (4M in dioxane, 1 mL, 109 mmol, 559 equiv) was stirred for 1 h, then concentrated under reduced pressure. The crude product was taken on without further purification. LCMS (ESI) m/z: [M+H] calcd for C₁₈H₁₆ClF₂N₄: 361.11; found 361.3.

### Step 4: Synthesis of (4-(6-chloro-8-fluoro-7-(2-fluorophenyl)quinazolin-4-yl)piperazin-1-yl)(1-tritylaziridin-2-yl)methanone

To a solution of 1-tritylaziridine-2-carboxylic acid (164 mg, 0.499 mmol, 1 equiv), 6-chloro-8-fluoro-7-(2-fluorophenyl)-4-(piperazin-1-yl)quinazoline (180 mg, 0.499 mmol, 1 equiv) and HOBt (3.36 mg, 0.0249 mmol, 0.05 equiv) in DMA (2.5 mL) was added NMM (119 μL, 1.09 mmol, 2.2 equiv) followed by EDC (104 mg, 0.549 mmol, 1.1 equiv). The resulting mixture was stirred for 2 h then diluted with EtOAc, washed with 1:1 H₂O/sat. aq. NaCl, dried over Na₂SO₄, filtered, and concentrated under reduced pressure. The resulting solid was purified by silica gel column chromatography (0%→100% EtOAc/hexanes) to afford (4-(6-chloro-8-fluoro-7-(2-fluorophenyl)quinazolin-4-yl)piperazin-1-yl)(1-tritylaziridin-2-yl)methanone. LCMS (ESI) m/z: [M+H] calcd for C₄₀H₃₃ClF₂N₅O: 672.23; found 672.3.

### Step 5: Synthesis of aziridin-2-yl(4-(6-chloro-8-fluoro-7-(2-fluorophenyl)quinazolin-4-yl)piperazin-1-yl)methanone

To a solution of (4-(6-chloro-8-fluoro-7-(2-fluorophenyl)quinazolin-4-yl)piperazin-1-yl)(1-tritylaziridin-2-yl)methanone (50 mg, 0.0744 mmol, 1 equiv) in MeOH (371 μL) and CHCl₃ (371 μL) at 0° C. was added TFA (45.5 μL, 0.595 mmol, 8 equiv) dropwise. The resulting mixture was stirred at 0° C. for 2 h then quenched with N,N-diisopropylethylamine (129 μL, 0.744 mmol, 10 equiv) and warmed to room temperature. The reaction was diluted with DCM, washed with sat. aq. NaCl, dried over Na₂SO₄, filtered, and concentrated under reduced pressure. The resulting solid was purified by reverse phase chromatography (10→100% MeCN/H₂O) to afford aziridin-2-yl(4-(6-chloro-8-fluoro-7-(2-fluorophenyl)quinazolin-4-yl)piperazin-1-yl)methanone (11 mg, 35% yield) as a white solid. LCMS (ESI) m/z: [M+H] calcd for C₂₁H₁₉ClF₂N₅O: 430.12; found 430.4. ¹H NMR (500 MHz, Methanol-d₄) δ 8.69 (s, 1H), 8.08 (d, J=1.7 Hz, 1H), 7.58 (tdd, J=7.6, 5.3, 1.8 Hz, 1H), 7.45 (td, J=7.4, 1.9 Hz, 1H), 7.37 (td, J=7.5, 1.1 Hz, 1H), 7.32 (dd, J=9.7, 8.4 Hz, 1H), 4.19-4.09 (m, 1H), 4.09-3.94 (m, 6H), 3.87 (t, J=5.3 Hz, 2H), 2.98 (dd, J=5.8, 3.4 Hz, 1H), 1.92 (d, J=5.8 Hz, 1H), 1.89 (d, J=3.3 Hz, 1H).

### Example 17—Synthesis of 1-(2-(4-(6-chloro-8-fluoro-7-(2-fluorophenyl)quinazolin-4-yl)piperazine-1-carbonyl)aziridin-1-yl)ethan-1-one

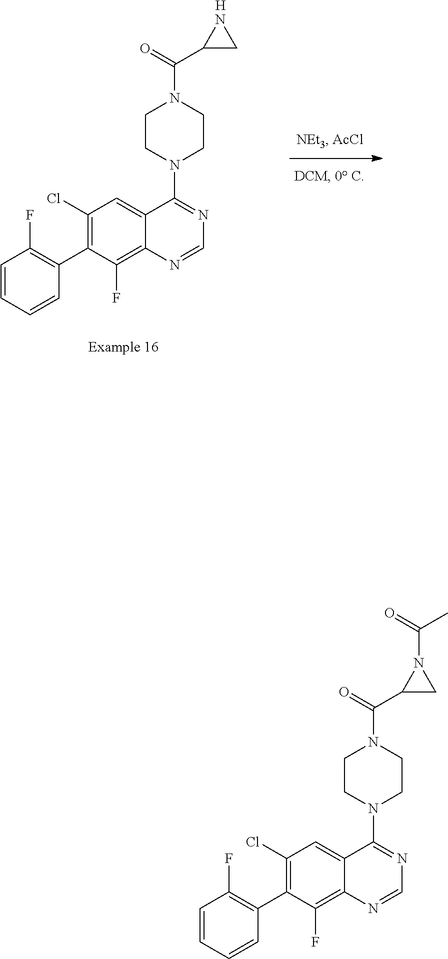

To a solution of aziridin-2-yl(4-(6-chloro-8-fluoro-7-(2-fluorophenyl)quinazolin-4-yl)piperazin-1-yl)methanone (20 mg, 0.0465 mmol, 1 equiv) in DCM (465 μL) at 0° C. was added NEt₃ (32.3 μL, 0.233 mmol, 5 equiv) followed by acetyl chloride (6.6 μL, 0.093 mmol, 2 equiv). The resulting mixture was stirred at 0° C. for 1 h then diluted with DCM, washed with sat. aq. NaHCO₃ followed by sat. aq. NaCl, dried over Na₂SO₄, filtered, and concentrated under reduced pressure. The resulting residue was purified by reverse phase chromatography (10→100% MeCN/H₂O) to afford 1-(2-(4-(6-chloro-8-fluoro-7-(2-fluorophenyl)quinazolin-4-yl)piperazine-1-carbonyl)aziridin-1-yl)ethan-1-one (14 mg, 62% yield) as a white solid. LCMS (ESI) m/z: [M+H] calcd for C₂₃H₂₁ClF₂N₅O₂: 472.14; found 472.2. ¹H NMR (500 MHz, Methanol-d₄) δ 8.69 (s, 1H), 8.09 (d, J=1.8 Hz, 1H), 7.59 (dddd, J=8.4, 7.3, 5.3, 1.8 Hz, 1H), 7.45 (td, J=7.4, 1.8 Hz, 1H), 7.38 (td, J=7.5, 1.1 Hz, 1H), 7.32 (ddd, J=9.6, 8.4, 1.0 Hz, 1H), 4.20-3.97 (m, 6H), 3.91 (ddt, J=13.7, 7.0, 3.5 Hz, 1H), 3.82 (ddt, J=13.4, 7.5, 3.9 Hz, 1H), 3.71 (dd, J=5.5, 3.1 Hz, 1H), 2.61 (dd, J=5.5, 1.8 Hz, 1H), 2.57 (dd, J=3.1, 1.8 Hz, 1H), 2.17 (s, 3H).

### Example 18—Synthesis of 1-(1-(6-chloro-8-fluoro-7-(2-fluorophenyl)quinazolin-4-yl)piperidin-4-yl)-2,2,2-trifluoroethan-1-one

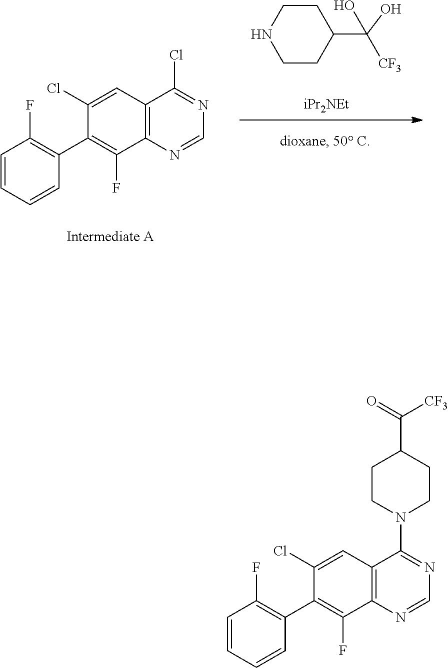 [SMILES: "iPr2NEt" ; Fc1ccccc1-c1c(Cl)cc2c(Cl)ncnc2c1F ; OC(O)(C1CCNCC1)C(F)(F)F ; O=C(C1CCN(c2ncnc3c(F)c(-c4ccccc4F)c(Cl)cc23)CC1)C(F)(F)F]

To a solution of 4,6-dichloro-8-fluoro-7-(2-fluorophenyl)quinazoline (70 mg, 0.22 mmol, 1 equiv) and 2,2,2-trifluoro-1-(piperidin-4-yl)ethane-1,1-diol hydrochloride (132 mg, 0.563 mmol, 2.5 equiv) in dioxane (2.3 mL) was added N,N-diisopropylethylamine (192 μL, 1.12 mmol, 5 equiv). The mixture was heated to 50° C. for 2.5 h and then concentrated under reduced pressure. The crude residue was purified by silica gel column chromatography (0→50% EtOAc/hexanes) to afford 1-(1-(6-chloro-8-fluoro-7-(2-fluorophenyl)quinazolin-4-yl)piperidin-4-yl)-2,2,2-trifluoroethan-1-one (29 mg, 27% yield) as a white powder. LCMS (ESI) m/z: [M+H₂O+H] calcd for C₂₁H₁₈ClF₅N₃O₂: 474.10; found 474.3. Observed as a 2:1 mixture of ketone/hydrate by NMR. ¹H NMR (ketone product) (500 MHz, DMSO-d₆) δ 8.71 (s, 1H), 8.00 (d, J=1.5 Hz, 1H), 7.62 (dddd, J=8.7, 7.4, 5.4, 1.8 Hz, 1H), 7.53 (tt, J=7.5, 1.7 Hz, 1H), 7.49-7.36 (m, 2H), 4.38 (d, J=13.0 Hz, 2H), 3.51-3.38 (m, 2H), 3.23 (t, J=12.2 Hz, 1H), 2.08 (d, J=13.5 Hz, 2H), 1.81 (qd, J=11.7, 3.9 Hz, 2H).

### Example 19—Synthesis of (1-(6-chloro-8-fluoro-7-(2-fluorophenyl)quinazolin-4-yl)piperidin-4-yl)boronic Acid

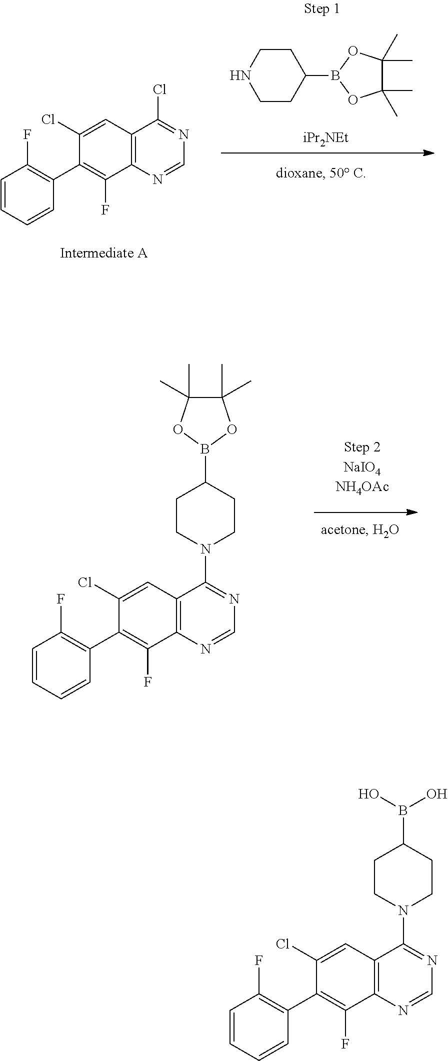 [SMILES: "iPr2NEt" ; Fc1ccccc1-c1c(Cl)cc2c(Cl)ncnc2c1F ; CC1(C)OB(C2CCNCC2)OC1(C)C ; CC1(C)OB(C2CCN(c3ncnc4c(F)c(-c5ccccc5F)c(Cl)cc34)CC2)OC1(C)C ; OB(O)C1CCN(c2ncnc3c(F)c(-c4ccccc4F)c(Cl)cc23)CC1]

### Step 1. Synthesis of 6-chloro-8-fluoro-7-(2-fluorophenyl-4-(4-(4,4,5,5-tetramethyl-1,3,2-dioxaborolan-2-yl)piperidin-1-yl)quinazoline

To a solution of 4,6-dichloro-8-fluoro-7-(2-fluorophenyl)quinazoline (102 mg, 0.327 mmol, 1 equiv) and 4-(4,4,5,5-tetramethyl-1,3,2-dioxaborolan-2-yl)piperidine (173 mg, 0.820 mmol, 2.5 equiv) in dioxane (3.3 mL) was added N,N-diisopropylethylamine (281 μL, 1.63 mmol, 5 equiv). The mixture was heated to 50° C. for 3 h and then concentrated under reduced pressure. The crude residue was purified by silica gel column chromatography (0→25% EtOAc/hexanes) to afford 6-chloro-8-fluoro-7-(2-fluorophenyl)-4-(4-(4,4,5,5-tetramethyl-1,3,2-dioxaborolan-2-yl)piperidin-1-yl)quinazoline (102 mg, 64%) as a foam. LCMS (ESI) m/z: [M+H] calcd for C₂₅H₂₈BClF₂N₃O₂: 486.20; found 486.4.

### Step 2. Synthesis of (1-(6-chloro-8-fluoro-7-(2-fluorophenyl)quinazolin-4-yl)piperidin-4-yl)boronic Acid

To a solution of 6-chloro-8-fluoro-7-(2-fluorophenyl)-4-(4-(4,4,5,5-tetramethyl-1,3,2-dioxaborolan-2-yl)piperidin-1-yl)quinazoline (85 mg, 0.17 mmol, 1 equiv) in acetone/H₂O (10:1, 874 μL) was added ammonium acetate (1M, 524 μL, 0.525 mmol, 3 equiv) and sodium periodate (112 mg, 0.525 mmol, 3 equiv). After 24 h the mixture was diluted with H₂O (500 μL), acidified with 1N HCl, and purified by reverse phase chromatography (10→99% MeCN/H₂O, 0.1% formic acid) to afford (1-(6-chloro-8-fluoro-7-(2-fluorophenyl)quinazolin-4-yl)piperidin-4-yl)boronic acid (34 mg, 48% yield) as a white powder. LCMS (ESI) m/z: [M+H] calcd for: C₁₉H₁₈BClF₂N₃O₂: 404.11; found 404.3. ¹H NMR (500 MHz, DMSO-d₆) δ 8.63 (s, 1H), 7.92 (d, J=1.5 Hz, 1H), 7.64-7.58 (m, 1H), 7.51 (dd, J=7.4, 1.8 Hz, 1H), 7.46-7.38 (m, 2H), 4.28 (dt, J=13.2, 3.7 Hz, 2H), 3.31-3.25 (m, 2H), 1.85-1.72 (m, 2H), 1.64 (qt, J=11.2, 3.6 Hz, 2H), 1.12 (tt, J=11.5, 3.9 Hz, 1H).

### Example 20—Synthesis of N-(2-methoxyethyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

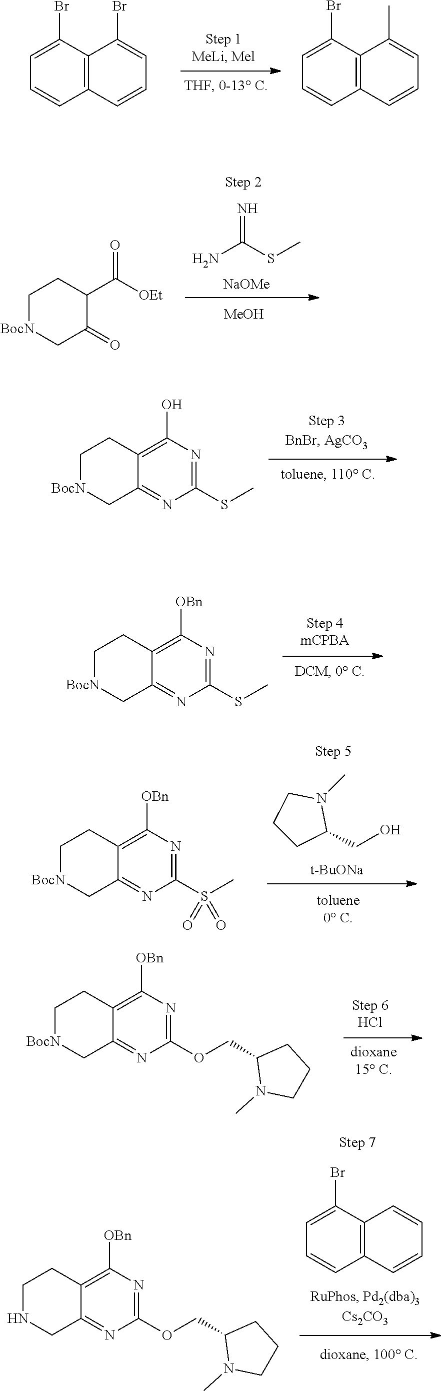

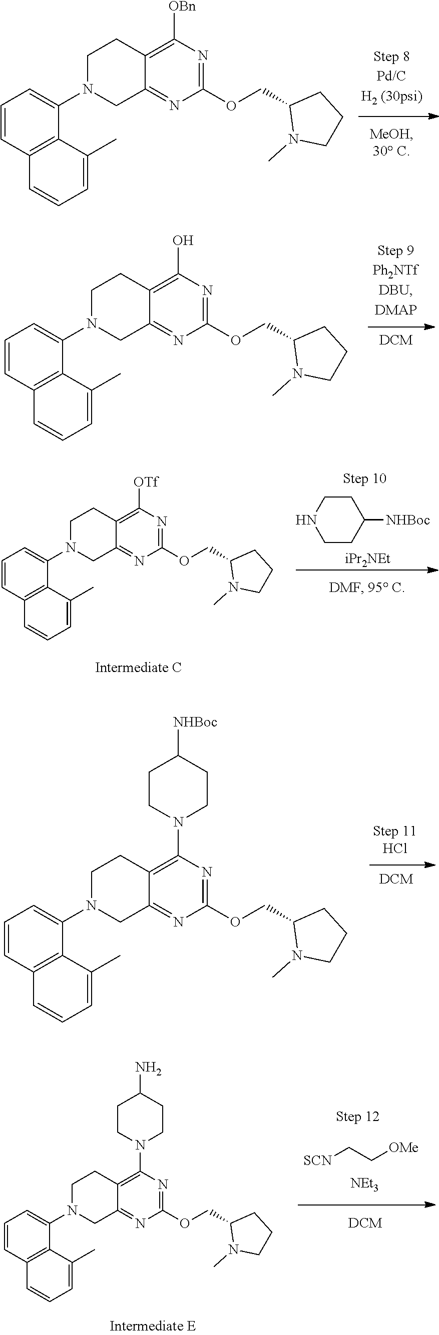

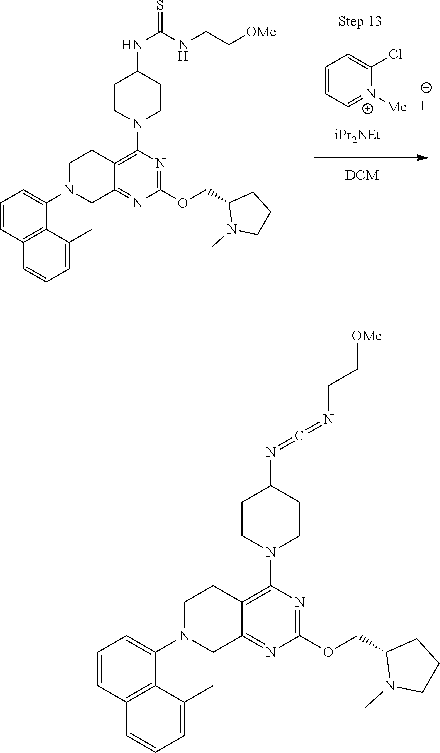

### Step 1: Synthesis of 1-bromo-8-methylnaphthalene

Two separate reactions were run in parallel. For each reaction, to a solution of 1,8-dibromonaphthalene (75 g, 262 mmol, 1 equiv) in THF (1.5 L) at 0° C. was added MeLi (1 M in 2-methyltetrahydrofuran, 420 mL, 1.6 equiv) dropwise, then the mixture was warmed to 13° C. After 0.5 h, MeI (253 g, 1.78 mol, 6.8 equiv) was added dropwise to the mixture. After 0.5 h, the two separate reaction mixtures were combined. H₂O (2 L) was poured into the mixture and the aqueous phase was extracted with EtOAc (2×800 mrL). The combined organic phase was washed with sat. aq. NaCl (800 mL), dried over Na₂SO₄, filtered and concentrated under reduced pressure. The residue was purified by silica gel column chromatography (100% petroleum ether) followed by reverse phase chromatography to afford 1-bromo-8-methyl-naphthalene (60 g, 51% yield) as a white solid. ¹H NMR (400 MHz, Chloroform-d) δ 7.84 (dd, J=1.2, 7.4 Hz, 1H), 7.79 (d, J=8.1 Hz, 1H), 7.75-7.68 (m, 1H), 7.39-7.33 (m, 2H), 7.22 (t, J=7.8 Hz, 1H), 3.14 (s, 3H).

### Step 2: Synthesis of tert-butyl 4-hydroxy-2-(methylthio)-5,6-dihydropyrido[3,4-d] pyrimidine-7(8H)-carboxylate

Five separate reactions were run in parallel. For each reaction, to a solution of 1-tert-butyl 4-ethyl 3-oxopiperidine-1,4-dicarboxylate (50 g, 184 mmol, 1 equiv) in MeOH (1 L) was added NaOMe (49.8 g, 921 mmol, 5 equiv) dropwise, followed by methyl carbamimidothioate sulfate (46.17 g, 332 mmol, 1.8 equiv). The reaction mixture was stirred at room temperature for 3 h, then the five separate reaction mixtures were combined, acidified with 2M HCl to pH 5, and concentrated under reduced pressure. The residue was suspended in EtOAc (1.5 L) and H₂O (1.5 L) and the mixture was stirred rapidly for 10 min. The resulting suspension was filtered, and the white solid was dried under vacuum. The mixture was azeotroped with anhydrous toluene (500 mL) then concentrated under reduced pressure to afford tert-butyl 4-hydroxy-2-(methylthio)-5,6-dihydropyrido[3,4-d]pyrimidine-7(8H)-carboxylate (250 g, 76% yield) as a white solid. ¹H NMR (400 MHz, Chloroform-d) δ 4.41-4.18 (m, 2H), 3.60 (s, 2H), 2.57 (s, 5H), 1.50 (s, 9H).

### Step 3: Synthesis of tert-butyl 4-(benzyloxy)-2-(methylthio)-5,6-dihydropyrido[3,4-d] pyrimidine-7(8H)-carboxylate

Three separate reactions were run in parallel. For each reaction to a solution of tert-butyl 4-hydroxy-2-(methylthio)-5,6-dihydropyrido[3,4-d]pyrimidine-7(8H)-carboxylate (60 g, 202 mmol, 1 equiv) in toluene (1.3 L) at 0° C. was added Ag₂CO₃ (44.5 g, 161 mmol, 0.8 equiv) and bromomethylbenzene (41.4 g, 242 mmol, 1.2 equiv). The resulting mixture was heated to 110° C. for 8 h then cooled to room temperature. The three separate reaction mixtures were combined, filtered and the solid cake was washed with EtOAc (2×500 mL) and H₂O (2×600 mL). The aqueous phase was extracted with EtOAc (3×500 mL) and the combined organic phase was washed with sat. aq. NaCl (1 L), dried over anhydrous Na₂SO₄, filtered and concentrated under reduced pressure. The residue was purified by silica gel column chromatography (0→15% EtOAc/petroleum ether) to afford tert-butyl 4-(benzyloxy)-2-(methylthio)-5,6-dihydropyrido[3,4-d] pyrimidine-7(8H)-carboxylate (230 g, 98% yield) as a white solid. ¹H NMR (400 MHz, Chloroform-d) δ 7.46-7.29 (m, 5H), 5.46 (s, 2H), 4.48 (s, 2H), 3.64 (t, J=5.6 Hz, 2H), 2.65 (s, 2H), 2.58-2.51 (m, 3H), 1.48 (s, 9H).

### Step 4: Synthesis of tert-butyl 4-(benzyloxy)-2-(methylsulfonyl)-5,6-dihydropyrido[3,4-d] pyrimidine-7(8H)-carboxylate

Two separate reactions were run in parallel. For each reaction, to a solution of tert-butyl 4-(benzyloxy)-2-(methylthio)-5,6-dihydropyrido[3,4-d] pyrimidine-7(8H)-carboxylate (75 g, 193 mmol, 1 equiv) in DCM (780 mL) at 0° C. was added m-CPBA (117 g, 542 mmol, 80% purity, 2.8 equiv) portion wise. The resulting mixture was stirred at 0° C. for 3 h. The two separate reaction mixtures were combined and quenched with sat. aq. Na₂SO₃ until no oxidant remained, as determined by KI starch paper. The organic phase was washed with sat. aq. NaCl (400 mL), dried over Na₂SO₄, filtered and concentrated under reduced pressure. The product was purified by silica gel column chromatography (0→25% EtOAc/petroleum ether) to afford tert-butyl 4-(benzyloxy)-2-(methylsulfonyl)-5,6-dihydropyrido[3,4-d] pyrimidine-7(8H)-carboxylate (100 g, 62% yield) as a white solid. ¹H NMR (400 MHz, Chloroform-d) δ 7.50-7.30 (m, 5H), 5.55 (s, 2H), 4.65 (s, 2H), 3.70 (t, J=5.6 Hz, 2H), 3.29 (s, 3H), 2.78 (s, 2H), 1.49 (s, 9H).

### Step 5: Synthesis of (S)-tert-butyl 4-(benzyloxy)-2-((1-methylpyrrolidin-2-yl)methoxy)-5,6-dihydropyrido[3,4-d]pyrimidine-7(8H)-carboxylate

Three separate reactions were run in parallel. For each reaction, to a solution of tert-butyl 4-(benzyloxy)-2-(methylsulfonyl)-5,6-dihydropyrido[3,4-d] pyrimidine-7(8H)-carboxylate (80 g, 191 mmol, 1 equiv) and (S)-(1-methylpyrrolidin-2-yl)methanol (43.9 g, 381 mmol, 2 equiv) in toluene (560 mL) at 0° C. was added t-BuONa (36.7 g, 381 mmol, 2 equiv). The resulting mixture was stirred at 0° C. for 10 min then the two separate reaction mixtures were combined, quenched with H₂O (1 L) and extracted into EtOAc (2×300 mL). The combined organic phase was washed with sat. aq. NaCl (300 mL), dried over Na₂SO₄, filtered and concentrated under reduced pressure. The residue was purified by silica gel column chromatography (0→10% MeOH/EtOAc) to afford crude product which was suspended in MTBE (800 mL) and stirred for 20 min. The mixture was filtered and the solid cake was washed with MTBE (3×50 mL), then the filtrate was concentrated under reduced pressure to afford (S)-tert-butyl 4-(benzyloxy)-2-((1-methylpyrrolidin-2-yl)methoxy)-5,6-dihydropyrido[3,4-d]pyrimidine-7(8H)-carboxylate (100 g, 46% yield) as a yellow liquid. ¹H NMR (400 MHz, Chloroform-d) δ 7.48-7.32 (m, 5H), 5.44 (s, 2H), 4.46 (s, 1H), 4.49-4.41 (m, 2H), 4.19 (m, 1H), 3.64 (t, J=5.4 Hz, 2H), 3.10 (t, J=7.5 Hz, 1H), 2.64 (m, 3H), 2.49 (s, 3H), 2.36-2.22 (m, 1H), 1.88-1.60 (m, 4H), 1.48 (s, 9H).

### Step 6: Synthesis of (S)-4-(benzyloxy)-2-((1-methylpyrrolidin-2-yl)methoxy)-5,6,7,8-tetrahydropyrido[3,4-d]pyrimidine

To a solution of (S)-tert-butyl 4-(benzyloxy)-2-((1-methylpyrrolidin-2-yl)methoxy)-5,6-dihydropyrido[3,4-d]pyrimidine-7(8H)-carboxylate (80 g, 176 mmol, 1 equiv) in dioxane (400 mL) was added HCl (4 M in dioxane, 1.10 L, 25 equiv). After 30 min the mixture was filtered and the solid cake was washed with MTBE (2×100 mL), then triturated with MTBE (300 mL) for 20 min. The mixture was filtered, and the solid cake was dried under reduced pressure. The solid was suspended in DCM (200 mL) and the pH was adjusted to pH 7-8 with sat. aq. NaHCO₃. The mixture was extracted into DCM (20×100 mL), dried over Na₂SO₄, filtered and concentrated under reduced pressure to afford (S)-4-(benzyloxy)-2-((1-methylpyrrolidin-2-yl)methoxy)-5,6,7,8-tetrahydropyrido[3,4-d] pyrimidine (52 g, 83% yield) as a red solid. ¹H NMR (400 MHz, Chloroform-d) δ 7.46-7.30 (m, 5H), 5.48-5.40 (s, 2H), 4.44 (dd, J=5.1, 10.8 Hz, 1H), 4.20 (dd, J=6.7, 10.7 Hz, 1H), 3.88 (s, 2H), 3.17 (t, J=7.6 Hz, 1H), 3.10 (t, J=5.9 Hz, 2H), 2.83-2.69 (m, 1H), 2.58 (t, J=5.8 Hz, 2H), 2.53 (s, 3H), 2.40-2.29 (m, 1H), 2.13-2.03 (m, 1H), 1.93-1.70 (m, 3H).

### Step 7: Synthesis of (S)-4-(benzyloxy)-7-(8-methylnaphthalen-1-yl)-2-((1-methylpyrrolidin-2-yl)methoxy)-5,6,7,8-tetrahydropyrido[3,4-d]pyrimidine

To a solution of (S)-4-(benzyloxy)-2-((1-methylpyrrolidin-2-yl)methoxy)-5,6,7,8-tetrahydropyrido[3,4-d]pyrimidine (20 g, 56.4 mmol, 1 equiv) in dioxane (400 mL) was added 1-bromo-8-methylnaphthalene (18.7 g, 84.6 mmol, 1.5 equiv), Cs₂CO₃ (46.0 g, 141 mmol, 2.5 equiv), RuPhos (5.27 g, 11.3 mmol, 0.2 equiv) and Pd₂(dba)₃ (5.17 g, 5.64 mmol, 0.1 equiv). The resulting mixture was heated to 100° C. for 13 h then cooled to room temperature. The mixture was filtered, and the solid cake was washed with DCM (3×80 mL), then the filtrate was concentrated under reduced pressure. The mixture was suspended in EtOAc (90 mL) and H₂O (90 mL). The aqueous phase was extracted into EtOAc (3×60 mL), the combined organic phase was washed with sat. aq. NaCl (50 mL), dried over Na₂SO₄, filtered and concentrated under reduced pressure. The residue was purified by silica gel column chromatography (100% EtOAc) to afford (S)-4-(benzyloxy)-7-(8-methylnaphthalen-1-yl)-2-((1-methylpyrrolidin-2-yl)methoxy)-5,6,7,8-tetrahydropyrido[3,4-d]pyrimidine (16 g, 44% yield) as a brown oil. ¹H NMR (400 MHz, Chloroform-d) δ 7.72-7.61 (m, 2H), 7.50-7.45 (m, 2H), 7.44-7.30 (m, 5H), 7.26-7.21 (m, 1H), 5.58-5.40 (m, 2H), 4.49-4.27 (m, 1H), 4.25-4.05 (m, 2H), 3.84 (d, J=17.4 Hz, 1H), 3.52 (dd, J=5.4, 11.9 Hz, 1H), 3.28-3.04 (m, 2H), 2.98-2.83 (s, 3H), 2.73 (d, J=16.6 Hz, 2H), 2.49 (s, 3H), 2.36-2.23 (m, 1H), 2.15-1.99 (m, 1H), 1.89-1.55 (m, 5H).

### Step 8: Synthesis of (S)-7-(8-methylnaphthalen-1-yl)-2-((1-methylpyrrolidin-2-yl)methoxy)-5,6,7,8-tetrahydropyrido[3,4-d]pyrimidin-4-ol

To a solution of (S)-4-(benzyloxy)-7-(8-methylnaphthalen-1-yl)-2-((1-methylpyrrolidin-2-yl)methoxy)-5,6,7,8-tetrahydropyrido[3,4-d]pyrimidine (20 g, 40.4 mmol, 1 equiv) in MeOH (400 mL) was added Pd/C (7 g, 10% purity). The mixture was stirred under H₂ (30 psi) at 30° C. for 1 h then filtered and the cake was washed with MeOH (5×100 mL). The solid cake was suspended in DCM (100 mL), and the mixture was stirred at 15° C. for 10 min then filtered. The cake was washed with DCM (5×50 mL) and the combined organic phase was concentrated under reduced pressure. The resulting crude product was triturated with MTBE (100 mL) for 20 min then dried under reduced pressure to afford (S)-7-(8-methylnaphthalen-1-yl)-2-((1-methylpyrrolidin-2-yl)methoxy)-5,6,7,8-tetrahydropyrido[3,4-d]pyrimidin-4-ol (10 g, 59% yield) as a white solid. LCMS (ESI) m/z: [M+H] calcd for C₂₄H₂₉N₄O₂: 405.22; found 405.2. ¹H NMR (400 MHz, Methanol-d₄) δ 7.66 (dd, J=7.5, 18.3 Hz, 2H), 7.41 (t, J=7.7 Hz, 1H), 7.35-7.29 (m, 2H), 7.26-7.20 (m, 1H), 4.48-4.40 (m, 1H), 4.39-4.30 (m, 1H), 3.89-3.81 (m, 1H), 3.65 (d, J=17.2 Hz, 1H), 3.48 (dd, J=6.0, 11.7 Hz, 1H), 3.25-3.13 (m, 2H), 2.91 (s, 1H), 2.88 (s, 3H), 2.77 (d, J=8.6 Hz, 1H), 2.63 (s, 1H), 2.57 (d, J=1.8 Hz, 3H), 2.56-2.46 (m, 1H), 2.17-2.04 (m, 1H), 1.94-1.69 (m, 3H).

### Step 9: Synthesis of (S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate

To a solution of (S)-7-(8-methylnaphthalen-1-yl)-2-((1-methylpyrrolidin-2-yl)methoxy)-5,6,7,8-tetrahydropyrido[3,4-d]pyrimidin-4-ol (1.5 g, 3.70 mmol, 1 equiv) in DCM (10 mL) was added DBU (553 μL, 3.70 mmol, 1 equiv), N-phenyl-bis(trifluoromethanesulfonimide) (1.98 g, 5.55 mmol, 1.5 equiv) and DMAP (9.04 mg, 0.074 mmol, 0.02 equiv). After 4 h, the reaction was concentrated under reduced pressure. The resulting residue was purified by silica gel column chromatography (0→70% EtOAc/hexanes, 1% NEt₃) to afford (S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate (1.5 g, 76% yield) as a brown oil. LCMS (ESI) m/z: [M+H] calcd for C₂₅H₂₇F₃N₄O₄S: 537.58; found 537.2.

### Step 10: Synthesis of tert-butyl (S)-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)carbamate

(S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate (500 mg, 931 μmol, 1 equiv), tert-butyl piperidin-4-ylcarbamate (372 mg, 1.86 mmol, 2.0 equiv) and N,N-diisopropylethylamine (485 μL, 2.79 mmol, 3.0 equiv) were added to DMF (5 mL). The reaction was heated to 95° C. for 3 h. The reaction was cooled to room temperature and diluted with H₂O (10 mL), the aqueous phase was washed with EtOAc (3×10 mL) and the organic layers were combined and washed with sat. aq. NaCl (10 mL). The organic layer was dried over Na₂SO₄, filtered, and concentrated under reduced pressure. The crude product was used without further purification. LCMS (ESI) m/z: [M+H] calcd for C₃₄H₄₇N₆O₃: 587.79; found 587.4.

### Step 11: Synthesis of (S)-1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-amine Hydrochloride

To a solution of tert-butyl (S)-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)carbamate (600 mg, 1.02 mmol, 1 equiv) in DCM (3 mL) was added HCl (4M in dioxane, 1.02 mL, 2 equiv). The reaction was stirred for 18 h and then concentrated under reduced pressure to afford (S)-1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-amine hydrochloride which was used without further purification. LCMS (ESI) m/z: [M+H] calcd for C₂₉H₃₉N₆O: 487.67; found 487.3.

### Step 12: Synthesis of (S)-1-(2-methoxyethyl)-3-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)thiourea

To a suspension of (S)-1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-amine hydrochloride (70 mg, 133 μmol, 1 equiv) in DCM (1.33 mL) was added NEt₃ (55.5 μL, 399 μmol, 3 equiv) followed by 1-isothiocyanato-2-methoxyethane (2-methoxyethyl isothiocyanate) (17.1 mg, 146 μmol, 1.1 equiv). The resulting mixture was stirred for 17 h then diluted with DCM (20 mL), washed with H₂O (10 mL) then sat. aq. NaCl (10 mL), dried over Na₂SO₄, filtered, and concentrated under reduced pressure to afford (S)-1-(2-methoxyethyl)-3-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)thiourea (53.1 mg, 66% yield over 3 steps) as a brown solid. LCMS (ESI) m/z: [M+H] calcd for C₃₃H₄₆N₇O₂S: 604.34; found 604.6.

### Step 13: Synthesis of N-(2-methoxyethyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

To a solution of (S)-1-(2-methoxyethyl)-3-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)thiourea (53.1 mg, 87.9 μmol, 1 equiv) in DCM (878 μL) was added N,N-diisopropylethylamine (45.6 μL, 263 μmol, 3 equiv) followed by 2-chloro-1-methylpyridin-1-ium iodide (33.4 mg, 131 μmol, 1.5 equiv). The resulting mixture was stirred for 16 h then filtered to remove solids and concentrated under reduced pressure. The crude residue was purified by reverse phase chromatography (40→100% MeCN/H₂O, 0.4% NH₄OH) to afford N-(2-methoxyethyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine (15.4 mg, 31% yield) as a pale brown oil. LCMS (ESI) m/z: [M+H] calcd for C₃₃H₄₄N₇O₂: 570.36; found 570.4; ¹H NMR (500 MHz, Methanol-d₄) δ 7.70 (d, J=8.3 Hz, 1H), 7.66 (dd, J=8.1, 1.3 Hz, 1H), 7.42 (t, J=7.7 Hz, 1H), 7.36-7.29 (m, 2H), 7.26 (d, J=7.0 Hz, 1H), 4.39 (ddd, J=12.9, 10.9, 6.1 Hz, 1H), 4.29 (ddd, J=16.6, 10.9, 5.8 Hz, 1H), 4.13-4.02 (m, 2H), 4.01-3.93 (m, 1H), 3.71-3.58 (m, 2H), 3.57-3.50 (m, 3H), 3.41-3.34 (m, 6H), 3.27-3.12 (m, 3H), 3.08 (dt, J=9.6, 4.5 Hz, 1H), 2.94 (s, 3H), 2.78-2.69 (m, 1H), 2.66-2.57 (m, 1H), 2.50 (d, J=1.1 Hz, 3H), 2.35 (q, J=9.0 Hz, 1H), 2.16-2.04 (m, 2H), 2.04-1.97 (m, 1H), 1.87-1.76 (m, 3H), 1.76-1.56 (m, 2H).

### Example 21—Synthesis of N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

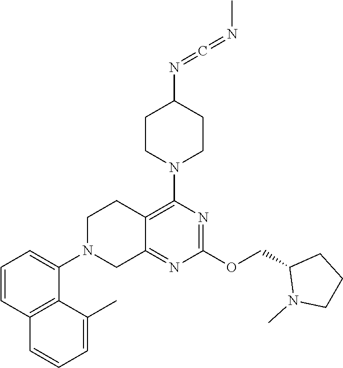 [SMILES: CN=C=NC1CCN(c2nc(OC[C@@H]3CCCN3C)nc3c2CCN(c2cccc4cccc(C)c24)C3)CC1]

Synthesized according to the method of example 20, using methyl isothiocyanate in place of 2-methoxyethyl isothiocyanate in step 12. LCMS (ESI) m/z: [M+H] calcd for C₃₁H₄₀N₇O: 526.71; found 526.3.

### Example 22—Synthesis of N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-N-propylmethanediimine

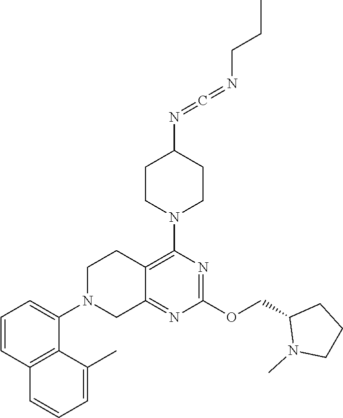 [SMILES: CCCN=C=NC1CCN(c2nc(OC[C@@H]3CCCN3C)nc3c2CCN(c2cccc4cccc(C)c24)C3)CC1]

Synthesized according to the method of example 20, using 1-propyl isothiocyanate in place of 2-methoxyethyl isothiocyanate in step 12. LCMS (ESI) m/z: [M+H] calcd for C₃₃H₄₄N₇O: 554.36; found 554.3.

### Example 23—Synthesis of N-isopropyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

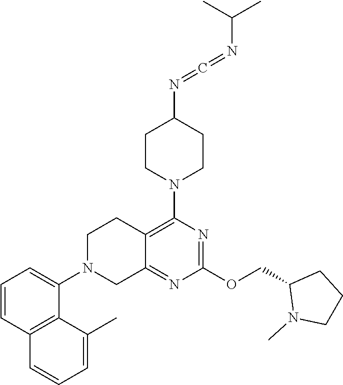 [SMILES: Cc1cccc2cccc(N3CCc4c(nc(OC[C@@H]5CCCN5C)nc4N4CCC(N=C=NC(C)C)CC4)C3)c12]

Synthesized according to the method of example 20, using 2-propyl isothiocyanate in place of 2-methoxyethyl isothiocyanate in step 12. LCMS (ESI) m/z: [M+H] calcd for C₃₃H₄₄N₇O: 554.36; found 554.3.

### Example 24—Synthesis of N-benzyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

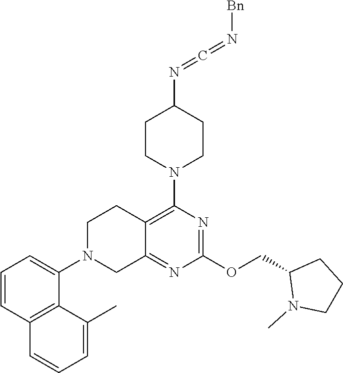 [SMILES: Cc1cccc2cccc(N3CCc4c(nc(OC[C@@H]5CCCN5C)nc4N4CCC(N=C=NCc5ccccc5)CC4)C3)c12]

Synthesized according to the method of example 20, using benzyl isothiocyanate in place of 2-methoxyethyl isothiocyanate in step 12. LCMS (ESI) m/z: [M+H] calcd for C₃₇H₄₆N₇O: 602.81; found 602.4.

### Example 25—Synthesis of N-(3-methoxypropyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

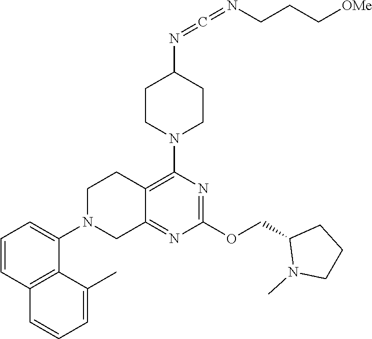 [SMILES: COCCCN=C=NC1CCN(c2nc(OC[C@@H]3CCCN3C)nc3c2CCN(c2cccc4cccc(C)c24)C3)CC1]

Synthesized according to the method of example 20, using 3-methoxypropyl isothiocyanate in place of 2-methoxyethyl isothiocyanate in step 12. LCMS (ESI) m/z: [M+H] calcd for C₃₄H₄₆N₇O₂: 584.37; found 584.4.

### Example 26—Synthesis of N-methyl-7-(8-methylnaphthalen-1-yl)-2-(((S)-1-methylpyrrolidin-2-yl)methoxy)-N-(2-(((propylimino)methylene)amino)ethyl)-5,6,7,8-tetrahydropyrido[3,4-d]pyrimidin-4-amine

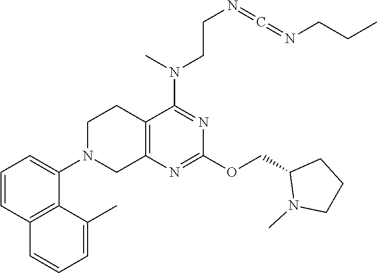 [SMILES: CCCN=C=NCCN(C)c1nc(OC[C@@H]2CCCN2C)nc2c1CCN(c1cccc3cccc(C)c13)C2]

Synthesized according to the method of example 20, using tert-butyl (2-(methylamino)ethyl)carbamate in place of tert-butyl piperidin-4-ylcarbamate in step 10, and 1-propyl isothiocyanate in place of 2-methoxyethyl in step 12. LCMS (ESI) m/z: [M+H] calcd for C₃₁H₄₂N₇O: 528.35; found 528.3.

### Example 27—Synthesis of N-(2-(((benzylimino)methylene)amino)ethyl)-N-methyl-7-(8-methylnaphthalen-1-yl)-2-(((S)-1-methylpyrrolidin-2-yl)methoxy)-5,6,7,8-tetrahydropyrido[3,4-d]pyrimidin-4-amine

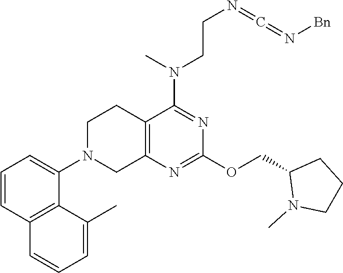 [SMILES: Cc1cccc2cccc(N3CCc4c(nc(OC[C@@H]5CCCN5C)nc4N(C)CCN=C=NCc4ccccc4)C3)c12]

Synthesized according to the method of example 20, using tert-butyl (2-(methylamino)ethyl)carbamate in place of tert-butyl piperidin-4-ylcarbamate in step 10, and benzyl isothiocyanate in place of 2-methoxyethyl isothiocyanate in step 12. LCMS (ESI) m/z: [M+H] calcd for C₃₅H₄₂N₇O: 576.77; found 576.5.

### Example 28—Synthesis of N-(2-((((4-chlorobenzyl)imino)methylene)amino)ethyl)-N-methyl-7-(8-methylnaphthalen-1-yl)-2-(((S)-1-methylpyrrolidin-2-yl)methoxy)-5,6,7,8-tetrahydropyrido[3,4-d]pyrimidin-4-amine

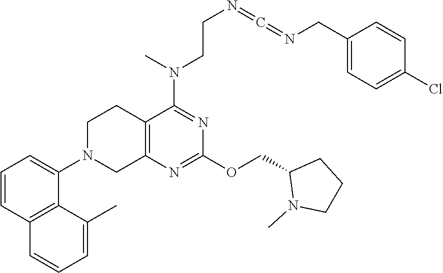 [SMILES: Cc1cccc2cccc(N3CCc4c(nc(OC[C@@H]5CCCN5C)nc4N(C)CCN=C=NCc4ccc(Cl)cc4)C3)c12]

Synthesized according to the method of example 20, using tert-butyl (2-(methylamino)ethyl)carbamate in place of tert-butyl piperidin-4-ylcarbamate in step 10, and 4-chlorobenzyl isothiocyanate in place of 2-methoxyethyl isothiocyanate in step 12. LCMS (ESI) m/z: [M+H] calcd for C₃₅H₄₁ClN₇O: 610.31; found 610.3.

### Example 29—Synthesis of N-(2-((((4-methoxybenzyl)imino)methylene)amino)ethyl)-N-methyl-7-(8-methylnaphthalen-1-yl)-2-(((S)-1-methylpyrrolidin-2-yl)methoxy)-5,6,7,8-tetrahydropyrido[3,4-d]pyrimidin-4-amine

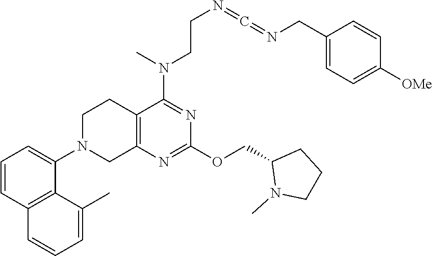 [SMILES: COc1ccc(CN=C=NCCN(C)c2nc(OC[C@@H]3CCCN3C)nc3c2CCN(c2cccc4cccc(C)c24)C3)cc1]

Synthesized according to the method of example 20, using tert-butyl (2-(methylamino)ethyl)carbamate in place of tert-butyl piperidin-4-ylcarbamate in step 10, and 4-methoxybenzyl isothiocyanate in place of 2-methoxyethyl isothiocyanate in step 12. LCMS (ESI) m/z: [M+H] calcd for C₃₆H₄₄N₇O₂: 606.36; found 606.4.

### Example 30—Synthesis of N-methyl-7-(8-methylnaphthalen-1-yl)-2-(((S)-1-methylpyrrolidin-2-yl)methoxy)-N-(3-(((propylimino)methylene)amino)propyl)-5,6,7,8-tetrahydropyrido[3,4-d]pyrimidin-4-amine

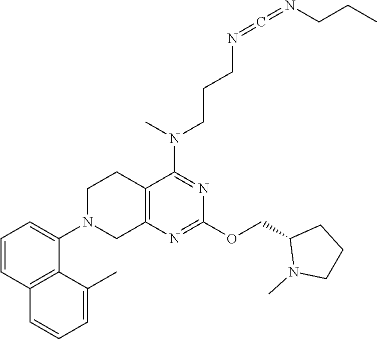 [SMILES: CCCN=C=NCCCN(C)c1nc(OC[C@@H]2CCCN2C)nc2c1CCN(c1cccc3cccc(C)c13)C2]

Synthesized according to the method of example 20, using tert-butyl (3-(methylamino)propyl)carbamate in place of tert-butyl piperidin-4-ylcarbamate in step 10 and 1-propyl isothiocyanate in place of 2-methoxyethyl in step 12. LCMS (ESI) m/z: [M+H] calcd for C₃₂H₄₄N₇O: 542.75; found 542.4.

### Example 31—Synthesis of N-(3-(((benzylimino)methylene)amino)propyl)-N-methyl-7-(8-methylnaphthalen-1-yl)-2-(((S)-1-methylpyrrolidin-2-yl)methoxy)-5,6,7,8-tetrahydropyrido[3,4-d]pyrimidin-4-amine

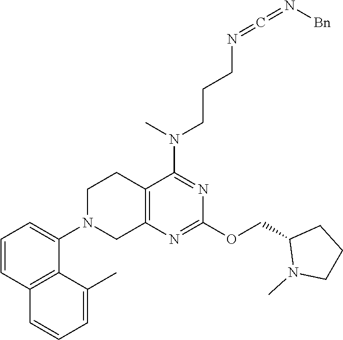 [SMILES: Cc1cccc2cccc(N3CCc4c(nc(OC[C@@H]5CCCN5C)nc4N(C)CCCN=C=NCc4ccccc4)C3)c12]

Synthesized according to the method of example 20, using tert-butyl (3-(methylamino)propyl)carbamate in place of tert-butyl piperidin-4-ylcarbamate in step 10, and benzyl isothiocyanate in place of 2-methoxyethyl isothiocyanate in step 12. LCMS (ESI) m/z: [M+H] calcd for C₃₆H₄₄N₇O: 590.36; found 590.3.

### Example 32—Synthesis of N-(3-((((4-chlorobenzyl)imino)methylene)amino)propyl)-N-methyl-7-(8-methylnaphthalen-1-yl)-2-(((S)-1-methylpyrrolidin-2-yl)methoxy)-5,6,7,8-tetrahydropyrido[3,4-d]pyrimidin-4-amine

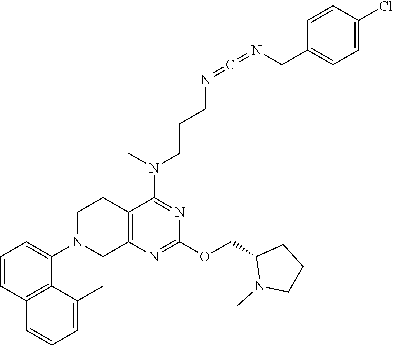 [SMILES: Cc1cccc2cccc(N3CCc4c(nc(OC[C@@H]5CCCN5C)nc4N(C)CCCN=C=NCc4ccc(Cl)cc4)C3)c12]

Synthesized according to the method of example 20, using tert-butyl (3-(methylamino)propyl)carbamate in place of tert-butyl piperidin-4-ylcarbamate in step 10, and 4-chlorobenzyl isothiocyanate in place of 2-methoxyethyl isothiocyanate in step 12. LCMS (ESI) m/z: [M+H] calcd for C₃₆H₄₃ClN₇O: 625.24; found 625.3.

### Example 33—Synthesis of N-(3-((((4-methoxybenzyl)imino)methylene)amino)propyl)-N-methyl-7-(8-methylnaphthalen-1-yl)-2-(((S)-1-methylpyrrolidin-2-yl)methoxy)-5,6,7,8-tetrahydropyrido[3,4-d]pyrimidin-4-amine

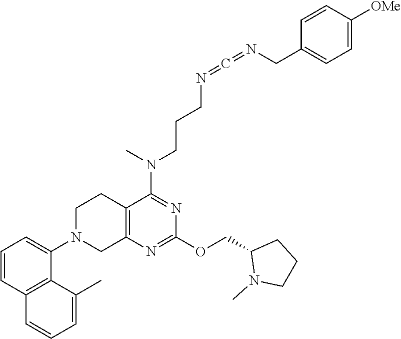 [SMILES: COc1ccc(CN=C=NCCCN(C)c2nc(OC[C@@H]3CCCN3C)nc3c2CCN(c2cccc4cccc(C)c24)C3)cc1]

Synthesized according to the method of example 20, using tert-butyl (3-(methylamino)propyl)carbamate in place of tert-butyl piperidin-4-ylcarbamate in step 10, and 4-methoxybenzyl isothiocyanate in place of 2-methoxyethyl isothiocyanate in step 12. LCMS (ESI) m/z: [M+H] calcd for C₃₇H₄₆N₇O₂: 620.82; found 620.4.

### Example 34—Synthesis of (S)—N-(2-chloroethyl)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carboxamide

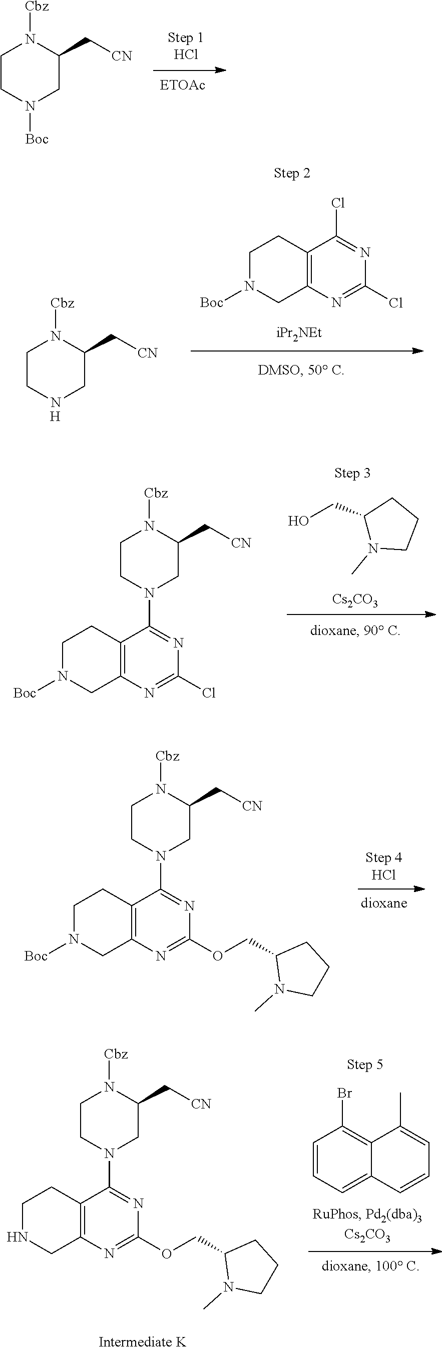

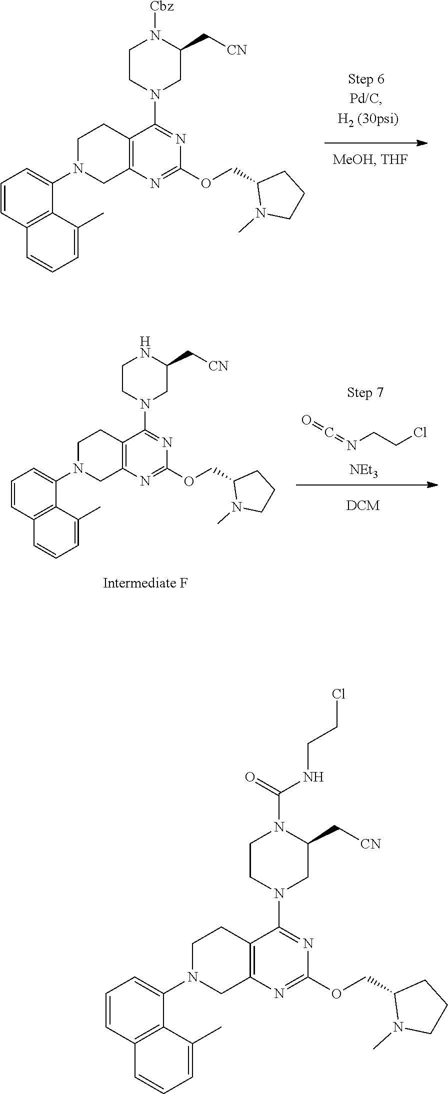

### Step 1: Synthesis of (S)-benzyl 2-(cyanomethyl)piperazine-1-carboxylate

To a solution of (S)-1-benzyl 4-tert-butyl 2-(cyanomethyl)piperazine-1,4-dicarboxylate (50 g, 139 mmol, 1 equiv) in EtOAc (500 mL) was added HCl (4 M in EtOAc, 174 mL, 5 equiv). After 12 h the reaction mixture was concentrated under reduced pressure to afford (S)-benzyl 2-(cyanomethyl)piperazine-1-carboxylate (41 g, HCl salt) as a yellow solid. LCMS (ESI) m/z: [M+H] calcd for C₁₄H₁₈N₃O₂: 260.13; found 260.2. ¹H NMR (400 MHz, Chloroform-d) δ 7.47-7.31 (m, 5H), 5.18 (s, 2H), 4.81 (s, 1H), 4.25 (br s, 1H), 3.66 (d, J=12.6 Hz, 1H), 3.56-3.32 (m, 3H), 3.18 (br d, J=11.6 Hz, 1H), 3.08-2.90 (m, 2H).

### Step 2: Synthesis of (S)-tert-butyl 4-(4-((benzyloxy)carbonyl)-3-(cyanomethyl)piperazin-1-yl)-2-chloro-5,6-dihydropyrido[3,4-d]pyrimidine-7(8H)-carboxylate

A solution of (S)-benzyl 2-(cyanomethyl)piperazine-1-carboxylate (41 g, 158 mmol, 1 equiv), tert-butyl 2,4-dichloro-5,6-dihydropyrido[3,4-d]pyrimidine-7(8H)-carboxylate (48 g, 158 mmol, 1 equiv), and N,N-diisopropylethylamine (41 g, 316 mmol, 55 mL, 2.0 equiv) in DMSO (410 mL) was heated to 50° C. After 3 h, the reaction mixture was cooled to room temperature and partitioned between EtOAc (500 mL) and sat. aq. NaCl (200 mL). The organic phase was washed with sat. aq. NaCl (3×300 mL), dried over Na₂SO₄, filtered and concentrated under reduced pressure. The residue was purified by silica gel chromatography (2→50% EtOAc/petroleum ether) to afford (S)-tert-butyl 4-(4-((benzyloxy)carbonyl)-3-(cyanomethyl)piperazin-1-yl)-2-chloro-5,6-dihydropyrido[3,4-d]pyrimidine-7(8H)-carboxylate (67 g, 79% yield) as a yellow solid. LCMS (ESI) m/z: [M+H] calcd for C₂₆H₃₂ClN₆O₄: 527.21; found 527.2. ¹H NMR (400 MHz, Chloroform-d) δ 7.39 (s, 5H), 5.20 (s, 2H), 4.72-4.60 (m, 2H), 4.50-4.39 (m, 1H), 4.18-4.02 (m, 2H), 3.88 (m, J=13.0 Hz, 2H), 3.39 (m, J=11.7 Hz, 3H), 3.11 (m, 1H), 2.88-2.58 (m, 4H), 1.49 (s, 9H).

### Step 3: Synthesis of tert-butyl 4-((S)-4-((benzyloxy)carbonyl)-3-(cyanomethyl)piperazin-1-yl)-2-(((S)-1-methylpyrrolidin-2-yl)methoxy)-5,6-dihydropyrido[3,4-d]pyrimidine-7(8H)-carboxylate

Two separate reactions were run in parallel. For each reaction, to a solution of (S)-tert-butyl 4-(4-((benzyloxy)carbonyl)-3-(cyanomethyl)piperazin-1-yl)-2-chloro-5,6-dihydropyrido[3,4-d]pyrimidine-7(8H)-carboxylate (28 g, 53 mmol, 1 equiv) and (S)-(1-methylpyrrolidin-2-yl)methanol (30.6 g, 266 mmol, 31.5 mL, 5.0 equiv) in dioxane (40 mL) was added Cs₂CO₃ (34.6 g, 106 mmol, 2 equiv), and the resulting mixture was heated to 90° C. After 12 h, the reaction was cooled to room temperature. The two separate reaction mixtures were combined and poured into H₂O (100 mL). The aqueous phase was extracted with DCM (2×200 mL). The combined organic phase was washed with sat. aq. NaCl (2×100 mL), dried over anhydrous Na₂SO₄, filtered and concentrated under reduced pressure. The resulting residue was purified by silica gel column chromatography (10→100% EtOAc/petroleum ether→20% MeOH/EtOAc) to afford tert-butyl 4-((S)-4-((benzyloxy)carbonyl)-3-(cyanomethyl)piperazin-1-yl)-2-(((S)-1-methylpyrrolidin-2-yl)methoxy)-5,6-dihydropyrido[3,4-d]pyrimidine-7(8H)-carboxylate (43 g, 57% yield) as a yellow solid. LCMS (ESI) m/z: [M+H] calcd for C₃₂H₄₄N₇O₅: 606.33; found 606.4. ¹H NMR (400 MHz, Methanol-d₄) δ 7.45-7.27 (m, 5H), 5.17-5.19 (m, 2H), 4.69 (br s, 1H), 4.55-4.45 (m, 1H), 4.39-4.22 (m, 3H), 4.16-3.91 (m, 4H), 3.82-3.70 (m, 1H), 3.38 (br d, J=8.8 Hz, 2H), 3.16-2.82 (m, 4H), 2.77-2.64 (m, 3H), 2.53-2.45 (s, 3H), 2.41 (m, 1H), 2.38-2.24 (m, 1H), 2.03 (m, 2H), 1.87-1.55 (m, 1H), 1.49 (s, 9H).

### Step 4: Synthesis of (S)-benzyl 2-(cyanomethyl)-4-(2-(((S)-1-methylpyrrolidin-2-ylmethoxy)-5,6,7,8-tetrahydropyrido[3,4-d]pyrimidin-4-yl)piperazine-1-carboxylate

To a solution of tert-butyl 4-((S)-4-((benzyloxy)carbonyl)-3-(cyanomethyl)piperazin-1-yl)-2-(((S)-1-methylpyrrolidin-2-yl)methoxy)-5,6-dihydropyrido[3,4-d]pyrimidine-7(8H)-carboxylate (33 g, 54.5 mmol, 1 equiv) in dioxane (150 mL) was added HCl (4 M in dioxane, 454 mL, 25 equiv). After 1 h the reaction was concentrated under reduced pressure. The resulting residue was poured into sat. aq. NaHCO₃ (100 mL) and the aqueous phase was extracted into DCM (5×100 mL). The combined organic phase was dried over anhydrous Na₂SO₄, filtered and concentrated under reduced pressure to afford (S)-benzyl 2-(cyanomethyl)-4-(2-(((S)-1-methylpyrrolidin-2-yl)methoxy)-5,6,7,8-tetrahydropyrido[3,4-d]pyrimidin-4-yl)piperazine-1-carboxylate (25 g, 90% yield) as a yellow solid. LCMS (ESI) m/z: [M+H] calcd for C₂₇H₃₆N₇O₃: 506.28; found 506.3. ¹H NMR (400 MHz, Methanol-d₄) δ 7.48-7.22 (m, 5H), 5.27-5.09 (m, 2H), 4.69 (s, 1H), 4.38-4.28 (m, 2H), 4.15-3.95 (m, 3H), 3.82 (s, 2H), 3.72-3.53 (m, 2H), 3.18-2.78 (m, 7H), 2.73-2.61 (m, 2H), 2.53 (s, 3H), 2.48-2.37 (m, 1H), 2.17-2.00 (m, 1H), 1.89-1.63 (m, 3H).

### Step 5: Synthesis of (S)-benzyl 2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carboxylate

To a solution of (S)-benzyl 2-(cyanomethyl)-4-(2-(((S)-1-methylpyrrolidin-2-yl)methoxy)-5,6,7,8-tetrahydropyrido[3,4-d]pyrimidin-4-yl)piperazine-1-carboxylate (20 g, 39.6 mmol, 1 equiv) in dioxane (300 mL) was added 1-bromo-8-methyl-naphthalene (13.1 g, 59.3 mmol, 1.5 equiv), Cs₂CO₃ (32.2 g, 99 mmol, 2.5 equiv), RuPhos (3.69 g, 7.91 mmol, 0.2 equiv) and Pd₂(dba)₃ (3.62 g, 4.0 mmol, 0.1 equiv) and the resulting mixture was heated to 100° C. After 12 h the reaction was cooled to room temperature, filtered, and concentrated under reduced pressure. The resulting residue was poured into H₂O (200 mL). The aqueous phase was extracted with DCM (3×300 mL) and the combined organic phase was washed with sat. aq. NaCl (2×100 mL), dried over anhydrous Na₂SO₄, filtered and concentrated under reduced pressure. The residue was purified by silica gel column chromatography (2→100% EtOAc/petroleum ether→20% MeOH/EtOAc) to afford (S)-benzyl 2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carboxylate (8.2 g, 30% yield) as a yellow solid. LCMS (ESI) m/z: [M+H] calcd for C₃₈H₄₄N₇O₃: 646.34; found 646.4. ¹H NMR (400 MHz, Methanol-d₄) δ 7.71-7.61 (m, 2H), 7.48-7.19 (m, 9H), 5.26-5.13 (m, 2H), 4.70 (br s, 1H), 4.38-4.25 (m, 2H), 4.20-3.98 (m, 3H), 3.77-3.62 (m, 1H), 3.57-3.39 (m, 2H), 3.26-3.14 (m, 3H), 3.13-3.01 (m, 2H), 2.90 (s, 3H), 2.84 (br s, 1H), 2.77-2.61 (m, 2H), 2.48 (d, J=4.5 Hz, 3H), 2.41-2.28 (m, 1H), 2.14-2.02 (m, 1H), 1.87-1.62 (m, 3H).

### Step 6: Synthesis of 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of (S)-benzyl 2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carboxylate (8.2 g, 12.70 mmol, 1 equiv) in MeOH (120 mL) and THF (120 mL) was added Pd/C (5 g, 10% purity) and the resulting mixture was stirred under H2 (30 psi). After 3 h the reaction mixture was filtered through celite, washed with MeOH (2×200 mL), and concentrated under reduced pressure. The residue was purified by reverse phase chromatography (40→60% MeCN/H₂O, 10 mM NH₄HCO₃) to afford 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (3.3 g, 50% yield) as a white solid. LCMS (ESI) m/z: [M+H] calcd for C₃₀H₃₈N₇O: 512.31; found 512.2. ¹H NMR (400 MHz, Methanol-d₄) δ 7.66 (dd, J=8.0, 17.3 Hz, 2H), 7.41 (dt, J=1.8, 7.7 Hz, 1H), 7.34-7.27 (m, 2H), 7.26-7.22 (m, 1H), 4.41-4.25 (m, 2.5H), 4.13-4.02 (m, 2H), 3.92 (d, J=11.9 Hz, 0.5H), 3.67 (dd, J=11.6, 17.8 Hz, 1H), 3.51 (m, 1H), 3.29-3.22 (m, 1H), 3.22-2.96 (m, 6H), 2.91 (s, 3H), 2.88-2.80 (m, 1H), 2.78-2.58 (m, 4H), 2.49 (d, J=1.6 Hz, 3H), 2.34 (q, J=8.9 Hz, 1H), 2.13-2.01 (m, 1H), 1.86-1.65 (m, 3H).

### Step 7: Synthesis of (S)—N-(2-chloroethyl)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carboxamide

To a solution of 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (30 mg, 58.6 μmol, 1 equiv) in THF (586 μL) was added NEt₃ (16.2 μL, 117 μmol, 2.0 equiv) followed by 1-chloro-2-isocyanatoethane (4.99 μL, 58.6 μmol, 1 equiv). The resulting mixture was stirred for 10 min then diluted with DCM, washed with H₂O, dried over Na₂SO₄, filtered, and concentrated under reduced pressure. The residue was purified by reverse phase chromatography (40→100% MeCN/H₂O, 0.1% NEt₃) to afford (S)—N-(2-chloroethyl)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carboxamide (13.8 mg, 38%) as a white solid. LCMS (ESI) m/z: [M+H] calcd for C₃₃H₄₂ClN₈O₂: 617.31; found 617.3. ¹H NMR (500 MHz, DMSO-d₆) δ 7.76 (d, J=8.3 Hz, 1H), 7.70 (t, J=6.9 Hz, 1H), 7.46 (dt, J=11.5, 7.7 Hz, 1H), 7.40-7.34 (m, 2H), 7.27 (d, J=7.8 Hz, 1H), 4.60-4.52 (m, 1H), 4.44-4.22 (m, 3H), 4.11-3.92 (m, 3H), 3.88-3.78 (m, 1H), 3.78-3.71 (m, 1H), 3.71-3.63 (m, 2H), 3.61 (t, J=6.6 Hz, 1H), 3.47-3.35 (m, 2H), 3.19-3.00 (m, 6H), 3.00-2.92 (m, 3H), 2.92-2.80 (m, 5H), 2.75-2.68 (m, 1H), 2.63-2.54 (m, 1H), 2.14-1.95 (m, 1H), 1.87-1.61 (m, 3H).

### Example 35—Synthesis of (S)—N-(2-chloroethyl)-4-(7-(8-methylnaphthalen-1-yl)-2-((1-methylpyrrolidin-2-yl)methoxy)-5,6,7,8-tetrahydropyrido[3,4-d]pyrimidin-4-yl)piperazine-1-carboxamide

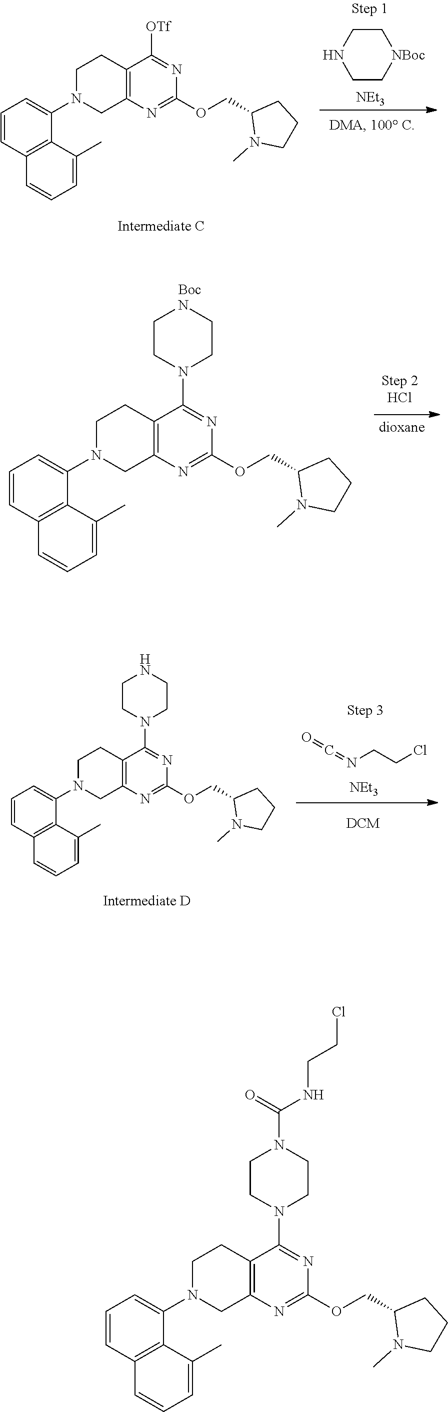

### Step 1: Synthesis of tert-butyl (S)-4-(7-(8-methylnaphthalen-1-yl)-2-((1-methylpyrrolidin-2-yl)methoxy)-5,6,7,8-tetrahydropyrido[3,4-d]pyrimidin-4-yl)piperazine-1-carboxylate

To a solution of (S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate (203 mg, 378 μmol, 1 equiv) in DMA (1 mL) was added tert-butyl piperazine-1-carboxylate (56 mg, 303 μmol, 0.8 equiv) followed by NEt₃ (52.6 μL, 378 μmol, 1 equiv). The resulting mixture was heated to 100° C. After 1 h the reaction was cooled to room temperature and concentrated under reduced pressure. The crude residue was purified by reverse phase chromatography (50→100% MeCN/H₂O, 0.1% NEt₃) to afford tert-butyl (S)-4-(7-(8-methylnaphthalen-1-yl)-2-((1-methylpyrrolidin-2-yl)methoxy)-5,6,7,8-tetrahydropyrido[3,4-d]pyrimidin-4-yl)piperazine-1-carboxylate. LCMS (ESI) m/z: [M+H] calcd for C₃₃H₄₆N₆O₃: 573.36; found 573.3.

### Step 2: Synthesis of (S)-7-(8-methylnaphthalen-1-yl)-2-((1-methylpyrrolidin-2-yl)methoxy)-4-(piperazin-1-yl)-5,6,7,8-tetrahydropyrido[3,4-d]pyrimidine Hydrochloride

To a solution of tert-butyl (S)-4-(7-(8-methylnaphthalen-1-yl)-2-((1-methylpyrrolidin-2-yl)methoxy)-5,6,7,8-tetrahydropyrido[3,4-d]pyrimidin-4-yl)piperazine-1-carboxylate (126 mg, 219 μmol) in MeOH (1.09 mL) was added HCl (4M in dioxane, 1.09 mL, 4.36 mmol, 20 equiv). The resulting mixture was stirred for 1 h then concentrated under reduced pressure. The crude product was taken on without further purification. LCMS (ESI) m/z: [M+H] calcd for C₂₈H₃₇N₆O: 473.31; found 473.3.

### Step 3: Synthesis of (S)—N-(2-chloroethyl)-4-(7-(8-methylnaphthalen-1-yl)-2-((1-methylpyrrolidin-2-yl)methoxy)-5,6,7,8-tetrahydropyrido[3,4-d]pyrimidin-4-yl)piperazine-1-carboxamide

Synthesized according to the method of example 34, step 7, using (S)-7-(8-methylnaphthalen-1-yl)-2-((1-methylpyrrolidin-2-yl)methoxy)-4-(piperazin-1-yl)-5,6,7,8-tetrahydropyrido[3,4-d]pyrimidine hydrochloride in place of 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile. LCMS (ESI) m/z: [M+H] calcd for C₃₁H₄₁ClN₇O₂: 578.30; found 578.3.

### Example 36—Synthesis of (S)-1-(2-chloroethyl)-3-(2-((7-(8-methylnaphthalen-1-yl)-2-((1-methylpyrrolidin-2-yl)methoxy)-5,6,7,8-tetrahydropyrido[3,4-d]pyrimidin-4-yl)amino)ethyl)urea

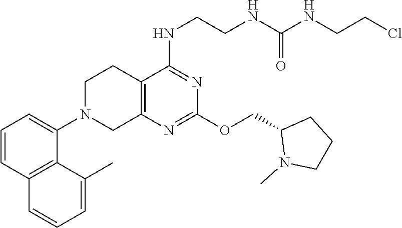 [SMILES: Cc1cccc2cccc(N3CCc4c(nc(OC[C@@H]5CCCN5C)nc4NCCNC(=O)NCCCl)C3)c12]

Synthesized according to the method of example 35, using tert-butyl (2-aminoethyl)carbamate in place of tert-butyl piperazine-1-carboxylate in step 1. LCMS (ESI) m/z: [M+H] calcd for C₂₉H₃₉ClN₇O₂: 552.29; found 552.3.

### Example 37—Synthesis of (S)-3-(2-chloroethyl)-1-methyl-1-(2-((7-(8-methylnaphthalen-1-yl)-2-((1-methylpyrrolidin-2-yl)methoxy)-5,6,7,8-tetrahydropyrido[3,4-d]pyrimidin-4-yl)amino)ethyl)urea

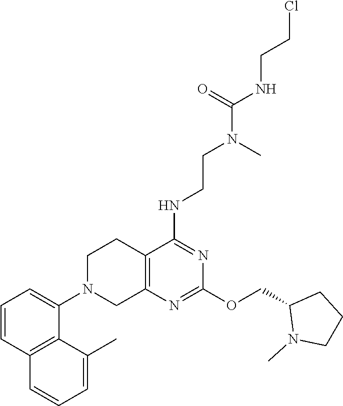 [SMILES: Cc1cccc2cccc(N3CCc4c(nc(OC[C@@H]5CCCN5C)nc4NCCN(C)C(=O)NCCCl)C3)c12]

Synthesized according to the method of example 35, using tert-butyl (2-aminoethyl)(methyl)carbamate in place of tert-butyl piperazine-1-carboxylate in step 1. LCMS (ESI) m/z: [M+H] calcd for C₃₀H₄₁ClN₇O₂: 566.30; found 566.3.

### Example 38—Synthesis of 2-((S)-1-(4,5-dihydrooxazol-2-yl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

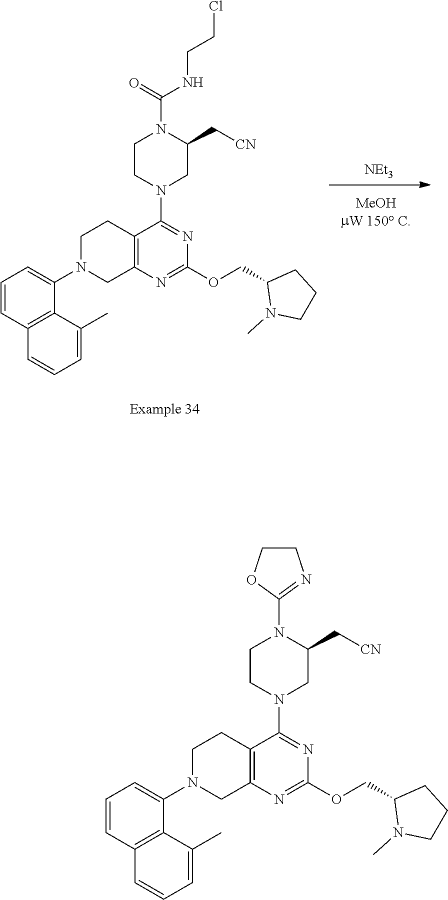 [SMILES: Cc1cccc2cccc(N3CCc4c(nc(OC[C@@H]5CCCN5C)nc4N4CCN(C(=O)NCCCl)[C@@H](CC#N)C4)C3)c12 ; Cc1cccc2cccc(N3CCc4c(nc(OC[C@@H]5CCCN5C)nc4N4CCN(C5=NCCO5)[C@@H](CC#N)C4)C3)c12 ; "NEt3"]

To a solution of (S)—N-(2-chloroethyl)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carboxamide (50 mg, 81.0 μmol) in MeOH (1.5 mL) was added N,N-diisopropylethylamine (58 μL, 332 μmol) and the solution was heated in the microwave to 150° C. for 45 seconds. The reaction mixture was concentrated under reduced pressure then purified by reverse phase chromatography (40→100% MeCN/H₂O, 0.1% NEt₃) to afford 2-((S)-1-(4,5-dihydrooxazol-2-yl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (8.5 mg, 18% yield) as a white solid. LCMS (ESI) m/z: [M+H] calcd for C₃₃H₄₁N₈O₂: 581.34; found 581.3. ¹H NMR (500 MHz, Methanol-d₄) δ 7.71 (d, J=8.1 Hz, 1H), 7.67 (d, J=8.0 Hz, 1H), 7.43 (t, J=7.7 Hz, 1H), 7.36-7.30 (m, 2H), 7.27 (d, J=7.2 Hz, 1H), 4.42-4.08 (m, 6H), 3.93-3.79 (m, 5H), 3.71 (dd, J=17.8, 2.5 Hz, 1H), 3.63 (dd, J=10.1, 4.2 Hz, 1H), 3.56 (d, J=7.4 Hz, 1H), 3.29-3.17 (m, 3H), 3.16-3.03 (m, 2H), 3.00-2.92 (m, 4H), 2.88 (dd, J=13.0, 11.0 Hz, 1H), 2.79-2.72 (m, 1H), 2.72-2.65 (m, 1H), 2.51 (d, J=3.0 Hz, 3H), 2.37 (q, J=9.0 Hz, 1H), 2.15-2.05 (m, 1H), 1.88-1.79 (m, 2H), 1.76-1.67 (m, 1H).

### Example 39—Synthesis of 2-((S)-1-(4,5-dihydrooxazol-2-yl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

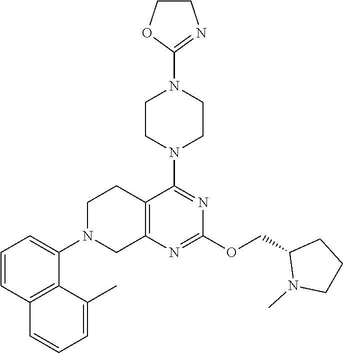 [SMILES: Cc1cccc2cccc(N3CCc4c(nc(OC[C@@H]5CCCN5C)nc4N4CCN(C5=NCCO5)CC4)C3)c12]

Synthesized according to the method of example 38, using example 35 in place of example 34. LCMS (ESI) m/z: [M+H] calcd for C₃₁H₄₀N₇O₂: 542.32; found 542.4.

### Example 40—Synthesis of (S)—N¹-(4,5-dihydrooxazol-2-yl)-N²-(7-(8-methylnaphthalen-1-yl)-2-((1-methylpyrrolidin-2-yl)methoxy)-5,6,7,8-tetrahydropyrido[3,4-d]pyrimidin-4-yl)ethane-1,2-diamine

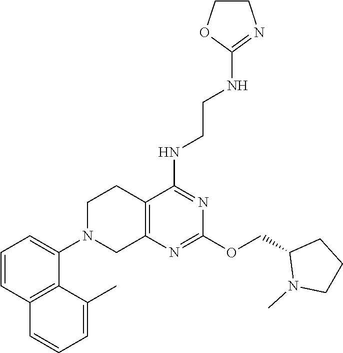 [SMILES: Cc1cccc2cccc(N3CCc4c(nc(OC[C@@H]5CCCN5C)nc4NCCNC4=NCCO4)C3)c12]

Synthesized according to the method of example 38, using example 36 in place of example 34. LCMS (ESI) m/z: [M+H] calcd for C₂₉H₃₈N₇O₂: 516.31; found 516.3.

### Example 41—Synthesis of (S)—N¹-(4,5-dihydrooxazol-2-yl)-N¹-methyl-N²-(7-(8-methylnaphthalen-1-yl)-2-((1-methylpyrrolidin-2-yl)methoxy)-5,6,7,8-tetrahydropyrido[3,4-d]pyrimidin-4-yl)ethane-1,2-diamine

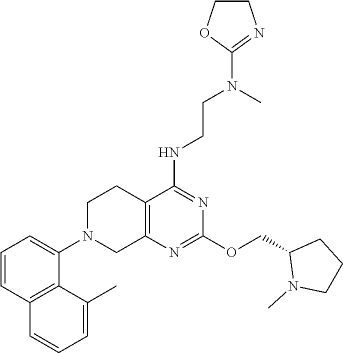 [SMILES: Cc1cccc2cccc(N3CCc4c(nc(OC[C@@H]5CCCN5C)nc4NCCN(C)C4=NCCO4)C3)c12]

Synthesized according to the method of example 38, using example 37 in place of example 34. LCMS (ESI) m/z: [M+H] calcd for C₃₀H₄₀N₇O₂: 530.33; found 530.4.

### Example 42—Synthesis of N-methyl-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)aziridine-2-carboxamide

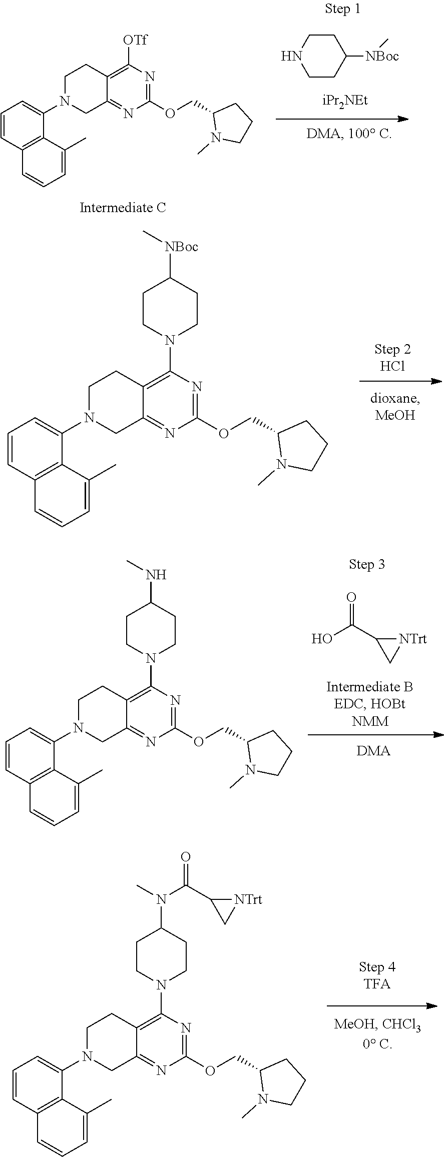

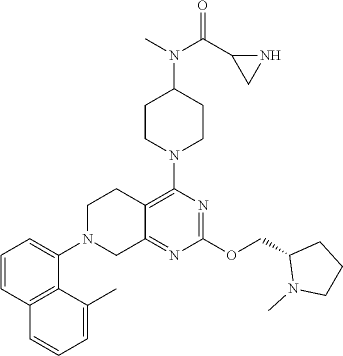 [SMILES: Cc1cccc2cccc(N3CCc4c(nc(OC[C@@H]5CCCN5C)nc4N4CCC(N(C)C(=O)C5CN5)CC4)C3)c12]

### Step 1: Synthesis of tert-butyl (S)-methyl(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)carbamate

Synthesized according to the method of example 35 step 1, using tert-butyl methyl(piperidin-4-yl)carbamate in place of tert-butyl piperazine-1-carboxylate.

### Step 2: Synthesis of (S)—N-methyl-1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-amine Hydrochloride

Synthesized according to the method of example 35 step 2, using tert-butyl (S)-methyl(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)carbamate in place of tert-butyl (S)-4-(7-(8-methylnaphthalen-1-yl)-2-((1-methylpyrrolidin-2-yl)methoxy)-5,6,7,8-tetrahydropyrido[3,4-d]pyrimidin-4-yl)piperazine-1-carboxylate. LCMS (ESI) m/z: [M+H] calcd for C₃₀H₄₁N₆O: 501.33; found 501.5.

### Step 3: Synthesis of N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-1-tritylaziridine-2-carboxamide

To a solution of 1-tritylaziridine-2-carboxylic acid (3.34 g, 3.58 mmol, 1.5 equiv), (S)—N-methyl-1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-amine (1.2 g, 2.39 mmol, 1 equiv), and HOBt (16.1 mg, 0.120 mmol, 0.05 equiv) in DMA (11.9 mL) was added NMM (2.61 mL, 23.9 mmol, 10 equiv) followed by EDC (1 g, 5.25 mmol, 2.2 equiv). The resulting mixture was stirred for 3 h then diluted with EtOAc, washed with 1:1 H₂O/sat. aq. NaCl, dried over Na₂SO₄, filtered, and concentrated under reduced pressure. The resulting residue was purified by silica gel column chromatography (0→20% MeOH/DCM) to afford N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-1-tritylaziridine-2-carboxamide (840 mg, 43% yield). LCMS (ESI) m/z: [M+H] calcd for C₅₂H₅₈N₇O₂: 821.47; found 812.7.

### Step 4: Synthesis of N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)aziridine-2-carboxamide

To a solution of N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-1-tritylaziridine-2-carboxamide (840 mg, 1.03 mmol, 1 equiv) in MeOH (5.15 ml) and CHCl₃ (5.15 mL) at 0° C. was added TFA (630 μL, 8.24 mmol, 8 equiv) dropwise. The resulting mixture was stirred for 2 h then quenched with lutidine (1.19 ml, 10.3 mmol, 10 equiv), diluted with DCM, washed with H₂O, dried over Na₂SO₄, filtered, and concentrated under reduced pressure. The resulting solid was purified by reverse phase chromatography (10→100% MeCN/H₂O) to afford of N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)aziridine-2-carboxamide (48 mg, 8% yield) as a white solid. LCMS (ESI) m/z: [M+H] calcd for C₃₃H₄₄N₇O₂: 570.36; found 570.5.

### Example 43—Synthesis of 1-acetyl-N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)aziridine-2-carboxamide

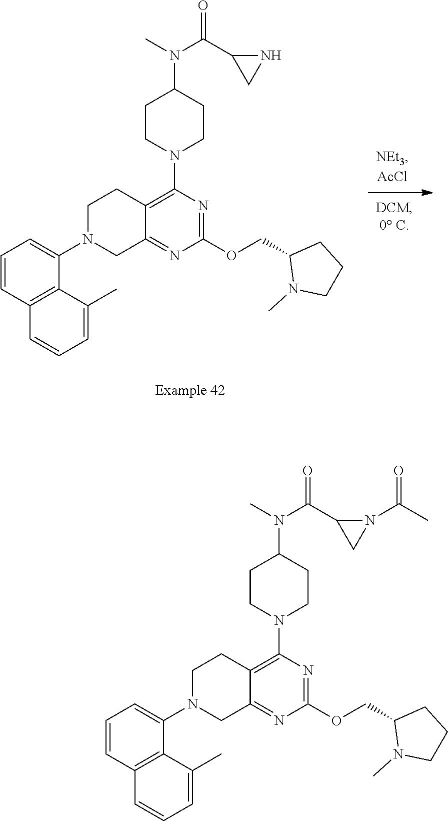 [SMILES: Cc1cccc2cccc(N3CCc4c(nc(OC[C@@H]5CCCN5C)nc4N4CCC(N(C)C(=O)C5CN5)CC4)C3)c12 ; CC(=O)N1CC1C(=O)N(C)C1CCN(c2nc(OC[C@@H]3CCCN3C)nc3c2CCN(c2cccc4cccc(C)c24)C3)CC1]

To a solution of N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)aziridine-2-carboxamide (13 mg, 0.023 mmol, 1 equiv) in DCM (456 μL) at 0° C. was added NEt₃ (15.8 μL, 0.11 mmol, 5 equiv) followed by acetyl chloride (3.25 μL, 0.046 mmol, 2 equiv). The resulting mixture was stirred at 0° C. for 1 h then diluted with DCM (5 mL), washed with NaHCO₃ (5 mL), dried over Na₂SO₄, filtered, and concentrated under reduced pressure. The resulting oil was lyophilized to afford 1-acetyl-N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)aziridine-2-carboxamide (7 mg, 51% yield) as a white solid. LCMS (ESI) m/z: [M+H] calcd for C₃₅H₄₆N₇O₃: 612.37; found 612.5.

### Example 44—Synthesis of N-methyl-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-1-(methylsulfonyl)aziridine-2-carboxamide

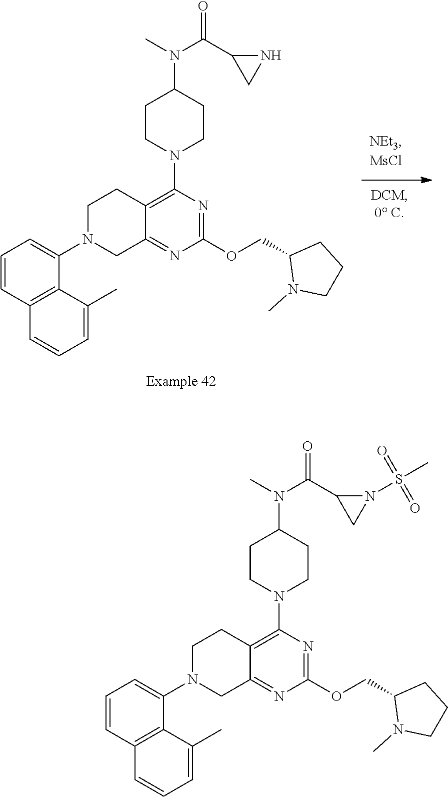 [SMILES: Cc1cccc2cccc(N3CCc4c(nc(OC[C@@H]5CCCN5C)nc4N4CCC(N(C)C(=O)C5CN5)CC4)C3)c12 ; Cc1cccc2cccc(N3CCc4c(nc(OC[C@@H]5CCCN5C)nc4N4CCC(N(C)C(=O)C5CN5S(C)(=O)=O)CC4)C3)c12]

To a solution of N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)aziridine-2-carboxamide (13 mg, 0.023 mmol, 1 equiv) in DCM (456 μL) at 0° C. was added NEt₃ (15.8 μL, 0.114 mmol, 5 equiv) followed by methanesulfonyl chloride (3.52 μL, 0.046 mmol, 2 equiv). The resulting mixture was stirred at 0° C. for 1 h then was diluted with DCM (5 mL), washed with NaHCO₃ (5 mL), dried over Na₂SO₄, filtered, and concentrated under reduced pressure. The resulting oil was lyophilized to afford N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-1-(methylsulfonyl)aziridine-2-carboxamide (8 mg, 53% yield) as a white solid. LCMS (ESI) m/z: [M+H] calcd for C₃₄H₄₆N₇O₄S: 648.33; found 648.5.

### Example 45—Synthesis of N,1-dimethyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)aziridine-2-carboxamide

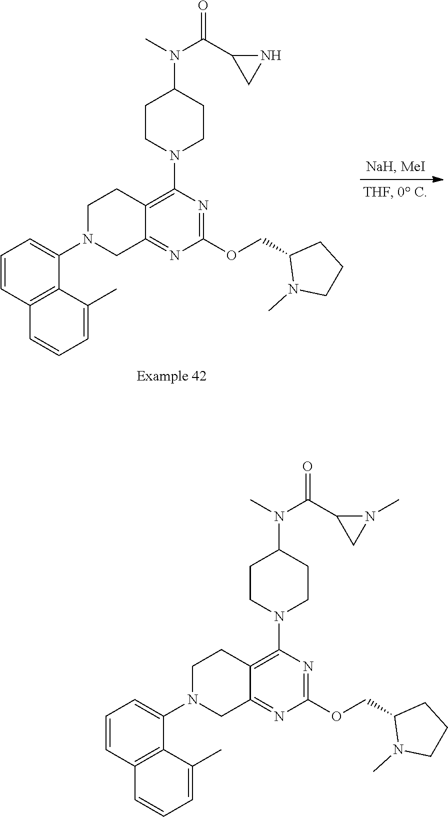 [SMILES: Cc1cccc2cccc(N3CCc4c(nc(OC[C@@H]5CCCN5C)nc4N4CCC(N(C)C(=O)C5CN5)CC4)C3)c12 ; Cc1cccc2cccc(N3CCc4c(nc(OC[C@@H]5CCCN5C)nc4N4CCC(N(C)C(=O)C5CN5C)CC4)C3)c12]

To a solution of N-methyl-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)aziridine-2-carboxamide (13 mg, 0.023 mmol) and MeI (7.1 μL, 0.11 mmol, 5 equiv) in THF (325 μL) at 0° C. was added NaH (656 μg, 0.027 mmol, 1.2 equiv). The resulting mixture was stirred at 0° C. for 3 h then warmed to room temperature and stirred for 24 h. The reaction was diluted with EtOAc, washed with 1:1 H₂O/sat. aq. NaCl, dried over Na₂SO₄, filtered, and concentrated under reduced pressure. The resulting oil was lyophilized to afford N,1-dimethyl-N-(1-(7-(8-methylnaphthalen-1-yl-2-(((S)-1-methylpyrrolidin-2-yl)methoxy)-5,6,7,8-tetrahydropyrido[3,4-d]pyrimidin-4-yl)piperidin-4-yl)aziridine-2-carboxamide (12 mg, 86% yield) as a white solid. LCMS (ESI) m/z: [M+H] calcd for C₃₄H₄₆N₇O₂: 584.37; found 584.5.

### Example 46—Synthesis of aziridin-2-yl(4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-1-yl)methanone

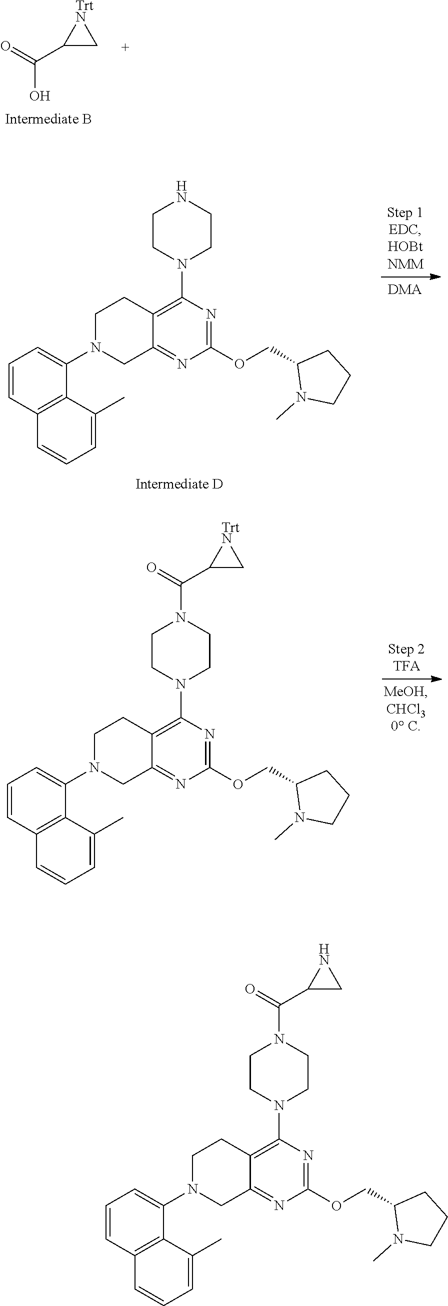

### Step 1: Synthesis of (4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-1-yl)(1-tritylaziridin-2-yl)methanone

To a solution of 1-tritylaziridine-2-carboxylic acid (1.56 g, 1.66 mmol, 1.5 equiv), (S)-7-(8-methylnaphthalen-1-yl)-2-((1-methylpyrrolidin-2-yl)methoxy)-4-(piperazin-1-yl)-5,6,7,8-tetrahydropyrido[3,4-d]pyrimidine hydrochloride (1.16 g, 1.11 mmol, 1 equiv), and HOBt (7.49 mg, 0.055 mmol, 0.05 equiv) in DMA (5.55 mL) was added NMM (1.21 mL, 11.1 mmol, 10 equiv) followed by EDC (466 mg, 2.44 mmol, 2.2 equiv). The resulting mixture was stirred for 18 h then diluted with EtOAc, washed with 1:1 H₂O/sat. aq. NaCl, dried over Na₂SO₄, filtered, and concentrated under reduced pressure. The resulting residue was purified by silica gel column chromatography (0→10% MeOH/DCM) to afford (4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-1-yl)(1-tritylaziridin-2-yl)methanone. LCMS (ESI) m/z: [M+H] calcd for C₅₀H₅₄N₇O₂: 784.43; found 784.7.

### Step 2: Synthesis of aziridin-2-yl(4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-1-yl)methanone

To a solution of (4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-1-yl)(1-tritylaziridin-2-yl)methanone (400 mg, 0.510 mmol, 1 equiv) in MeOH (2.55 mL) and CHCl₃ (2.55 mL) at 0° C. was added TFA (312 μL, 4.08 mmol, 8 equiv) dropwise. The resulting mixture was stirred for 1 h, warmed to room temperature, and stirred for 1 h. The reaction was quenched with N,N-diisopropylethylamine (888 μL, 5.10 mmol, 10 equiv), diluted with DCM, washed with sat. aq. NaCl, dried over Na₂SO₄, filtered, and concentrated under reduced pressure. The resulting solid was purified by reverse phase chromatography (10→100% MeCN/H₂O) to afford aziridin-2-yl(4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-1-yl)methanone (55 mg, 20% yield) as a white solid. LCMS (ESI) m/z: [M+H] calcd for C₃₁H₄₀N₇O₂: 542.32; found 542.5.

### Example 47—Synthesis of 1-(2-(4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl)aziridin-1-yl)ethan-1-one

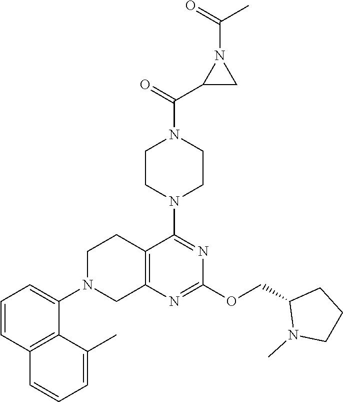 [SMILES: CC(=O)N1CC1C(=O)N1CCN(c2nc(OC[C@@H]3CCCN3C)nc3c2CCN(c2cccc4cccc(C)c24)C3)CC1]

Synthesized according to the method of example 43, using example 46 in place example 42. LCMS (ESI) m/z: [M+H] calcd for C₃₃H₄₂N₇O₃: 584.33; found 584.5.

### Example 48—Synthesis of N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-N-phenylmethanediimine

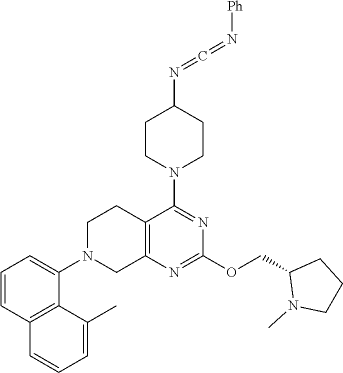 [SMILES: Cc1cccc2cccc(N3CCc4c(nc(OC[C@@H]5CCCN5C)nc4N4CCC(N=C=Nc5ccccc5)CC4)C3)c12]

Synthesized according to the method of example 20, using phenyl isothiocyanate in place of 2-methoxyethyl isothiocyanate in step 12. LCMS (ESI) m/z: [M+H] calcd for C₃₆H₄₂N₇O: 588.35; found 588.4.

### Example 49—Synthesis of N-(4-chlorobenzyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

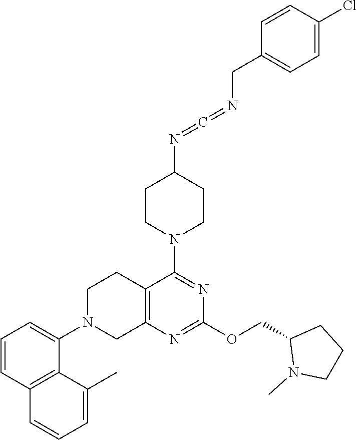 [SMILES: Cc1cccc2cccc(N3CCc4c(nc(OC[C@@H]5CCCN5C)nc4N4CCC(N=C=NCc5ccc(Cl)cc5)CC4)C3)c12]

Synthesized according to the method of example 20, using 4-chlorophenyl isothiocyanate in place of 2-methoxyethyl isothiocyanate in step 12. LCMS (ESI) m/z: [M+H] calcd for C₃₇H₄₃ClN₇O: 636.32; found 636.3.

### Example 50—Synthesis of N-(4-methoxybenzyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

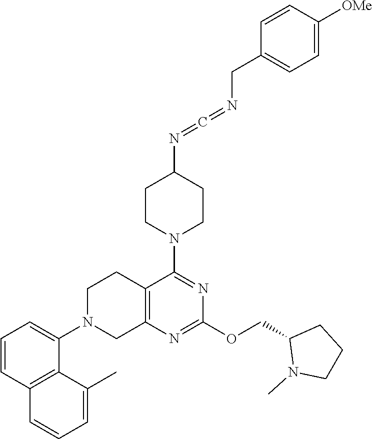 [SMILES: COc1ccc(CN=C=NC2CCN(c3nc(OC[C@@H]4CCCN4C)nc4c3CCN(c3cccc5cccc(C)c35)C4)CC2)cc1]

Synthesized according to the method of example 20, using 4-methoxyphenyl isothiocyanate in place of 2-methoxyethyl isothiocyanate in step 12. LCMS (ESI) m/z: [M+H] calcd for C₃₈H₄₆N₇O₂: 632.37; found 632.4.

### Example 51—Synthesis of (S)-2,2,2-trifluoro-1-(1-(7-(8-methylnaphthalen-1-yl)-2-((1-methyl pyrrolidin-2-yl)methoxy)-5,6,7,8-tetrahydropyrido[3,4-d]pyrimidin-4-yl)piperidin-4-yl)ethane-1,1-diol

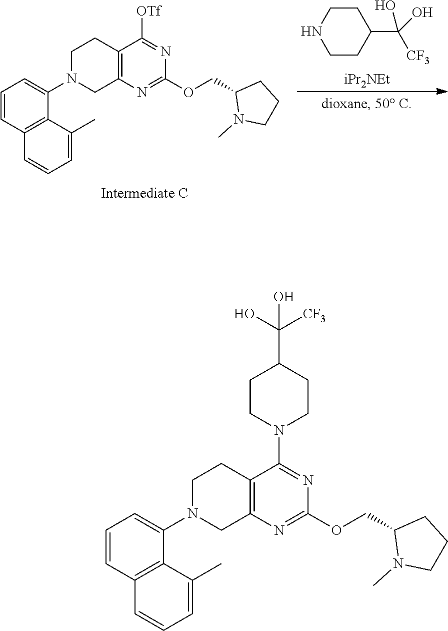 [SMILES: "iPr2NEt" ; Cc1cccc2cccc(N3CCc4c(nc(OC[C@@H]5CCCN5C)nc4OS(=O)(=O)C(F)(F)F)C3)c12 ; OC(O)(C1CCNCC1)C(F)(F)F ; Cc1cccc2cccc(N3CCc4c(nc(OC[C@@H]5CCCN5C)nc4N4CCC(C(O)(O)C(F)(F)F)CC4)C3)c12]

To a solution of (S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate (188 mg, 0.350 mmol, 1 equiv) and 2,2,2-trifluoro-1-(piperidin-4-yl)ethane-1,1-diol hydrochloride (206 mg, 0.876 mmol, 2.5 equiv) in dioxane (3.50 mL) was added N,N-diisopropylethylamine (302 μL, 1.75 mmol, 5 equiv). The mixture was heated to 50° C. for 24 h and then concentrated under reduced pressure. The crude residue was dissolved in EtOAc, washed with H₂O then sat. aq. NaCl, dried over MgSO₄, filtered and concentrated under reduced pressure. The residue was purified by column chromatography (0→10% MeOH/DCM) to afford (S)-2,2,2-trifluoro-1-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)ethane-1,1-diol (35 mg, 17% yield) as an off white powder. LCMS (ESI) m/z: [M+H] calcd for C₃₁H₃₉F₃N₅O₃: 586.30; found 586.5.

### Example 52—Synthesis of (S)-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)boronic Acid

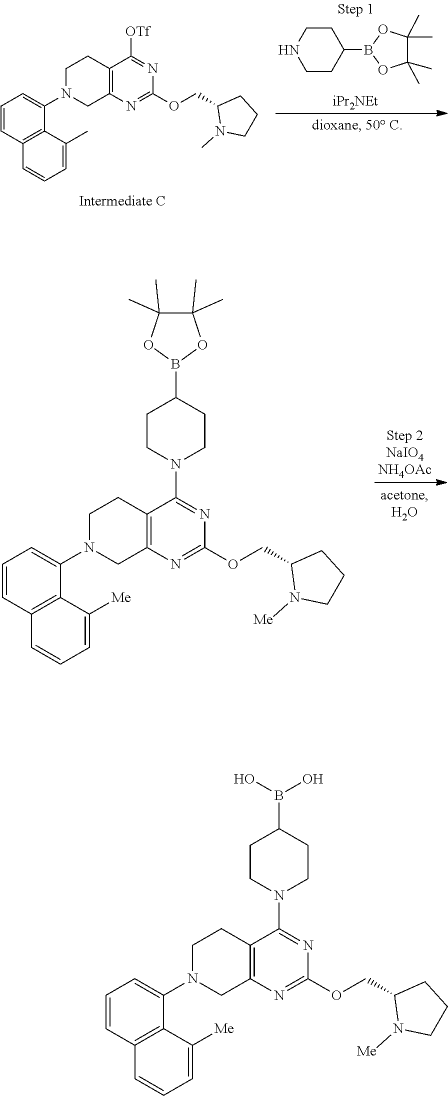 [SMILES: "iPr2NEt" ; Cc1cccc2cccc(N3CCc4c(nc(OC[C@@H]5CCCN5C)nc4OS(=O)(=O)C(F)(F)F)C3)c12 ; CC1(C)OB(C2CCNCC2)OC1(C)C ; Cc1cccc2cccc(N3CCc4c(nc(OC[C@@H]5CCCN5C)nc4N4CCC(B5OC(C)(C)C(C)(C)O5)CC4)C3)c12 ; Cc1cccc2cccc(N3CCc4c(nc(OC[C@@H]5CCCN5C)nc4N4CCC(B(O)O)CC4)C3)c12]

### Step 1: Synthesis of (S)-7-(8-methylnaphthalen-1-yl)-2-((1-methylpyrrolidin-2-yl)methoxy)-4-(4-(4,4,5,5-tetramethyl-1,3,2-dioxaborolan-2-yl)piperidin-1-yl)-5,6,7,8-tetrahydropyrido[3,4-d]pyrimidine

To a solution of (S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate (272 mg, 0.507 mmol, 1 equiv) and 4-(4,4,5,5-tetramethyl-1,3,2-dioxaborolan-2-yl)piperidine (213 mg, 1.01 mmol, 2 equiv), in dioxane (5.1 mL) was added N,N-diisopropylethylamine (436 μL, 2.53 mmol, 5 equiv). The mixture was heated to 50° C. for 2 h then concentrated under reduced pressure. The crude residue was purified by silica gel column chromatography (0→10% MeOH/DCM) to afford (S)-7-(8-methylnaphthalen-1-yl)-2-((1-methylpyrrolidin-2-yl)methoxy)-4-(4-(4,4,5,5-tetramethyl-1,3,2-dioxaborolan-2-yl)piperidin-1-yl)-5,6,7,8-tetrahydropyrido[3,4-d]pyrimidine (189 mg, 62% yield) as an oil. LCMS (ESI) m/z: [M+H] calcd for C₃₅H₄₉BN₅O₃: 598.40; found 598.6.

### Step 2: Synthesis of (S)-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)boronic Acid

To a solution of (S)-7-(8-methylnaphthalen-1-yl)-2-((1-methylpyrrolidin-2-yl)methoxy)-4-(4-(4,4,5,5-tetramethyl-1,3,2-dioxaborolan-2-yl)piperidin-1-yl)-5,6,7,8-tetrahydropyrido[3,4-d]pyrimidine (189 mg, 0.316 mmol, 1 equiv) in acetone/H₂O (10:1, 1.6 mL) was added ammonium acetate (1M. 948 μL. 0.949 mmol, 3 equiv) and sodium periodate (202 mg, 0.949 mmol, 3 equiv). The resulting mixture was stirred for 4 h then purified by reverse phase chromatography (10→99% MeCN/H₂O, 0.1% formic acid) to afford (S)-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)boronic acid (38 mg, 23% yield) as a white powder. LCMS (ESI) m/z: [M+H] calcd for C₂₉H₃₉BN₅O₃: 516.32; found 516.5. ¹H NMR observed as a 2:1 mixture of boronate and boronic acid. ¹H NMR (400 MHz, DMSO-d₆) δ 7.74-7.58 (m, 2H), 7.47-7.38 (m, 1H), 7.36-7.25 (m, 2H), 7.24-7.17 (m, 1H), 4.22 (ddd, J=11.1, 5.1, 1.8 Hz, 1H), 4.02 (dd, J=11.4, 6.3 Hz, 1H), 3.97-3.71 (m, 3H), 3.67-3.47 (m, 1H), 3.43-3.34 (m, 1H), 3.09-2.88 (m, 4H), 2.83 (s, 3H), 2.78 (dt, J=9.8, 4.4 Hz, 2H), 2.71-2.60 (m, 1H), 2.36 (d, J=2.0 Hz, 3H), 2.31-2.20 (m, 1H), 1.91 (dq, J=12.3, 8.4, 7.9 Hz, 1H), 1.73-1.49 (m, 6H), 1.40 (q, J=11.3 Hz, 1H), 1.04-0.87 (m, 0.67H), 0.83-0.73 (m, 0.33H).

### Example 53—Synthesis of 6-fluoro-7-(2-fluorophenyl)-1-(2-isopropyl-4-methylpyridin-3-yl)-4-(4-(((propylimino)methylene)amino)piperidin-1-yl)pyrido[2,3-d]pyrimidin-2(1H)-one

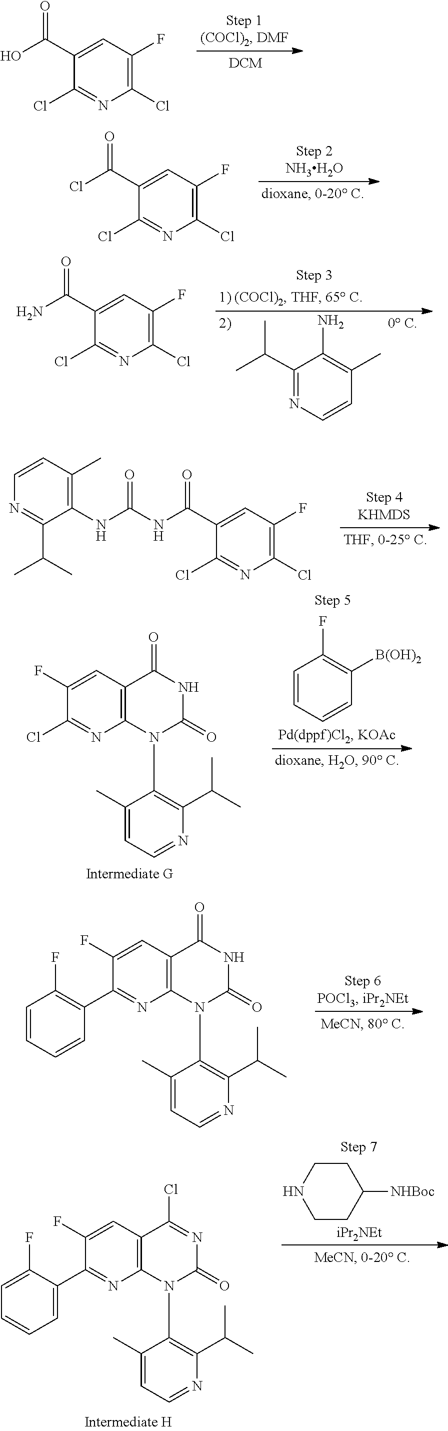

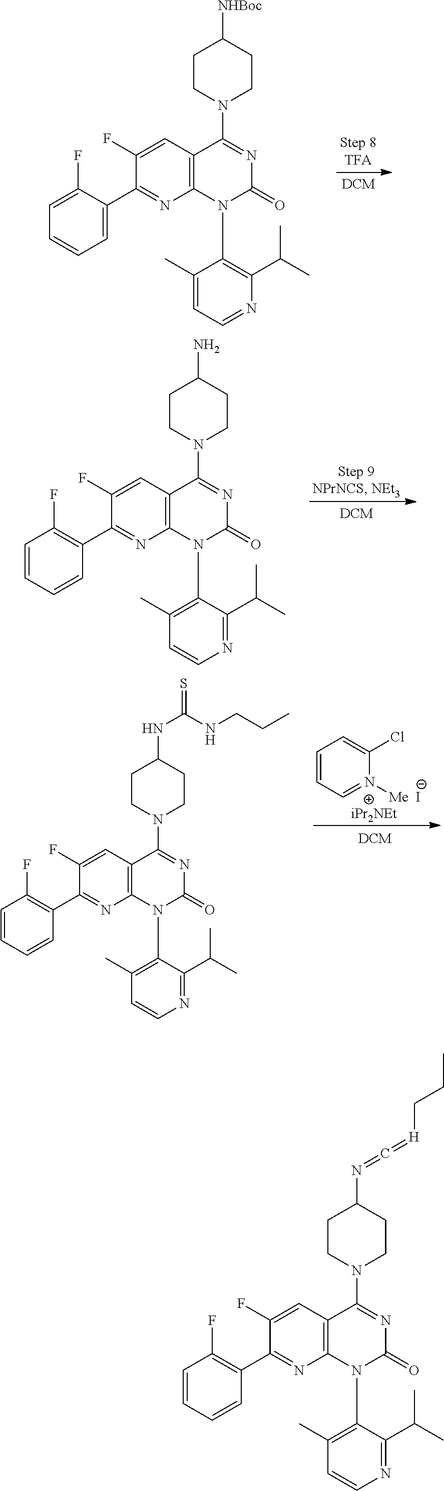

### Step 1: Synthesis of 2,6-dichloro-5-fluoronicotinoyl Chloride

To a solution of 2,6-dichloro-5-fluoronicotinic acid (100 g, 476 mmol, 1 equiv) in DCM (1 L) was added (COCl)₂ (72.5 g, 571 mmol, 1.2 equiv) followed by DMF (1 mL). The resulting mixture was stirred for 3 h then concentrated under reduced pressure to afford 2,6-dichloro-5-fluoronicotinoyl chloride as a pale-yellow liquid, which was used without further purification.

### Step 2: Synthesis of 2,6-dichloro-5-fluoronicotinamide

To a solution of 2,6-dichloro-5-fluoronicotinoyl chloride (109 g, 476 mmol, 1 equiv) in dioxane (1 L) at 0° C. was added NH₃.H₂O (133 g, 952 mmol, 25% w/w, 2 equiv). The resulting mixture was warmed to room temperature, stirred for 1 h then concentrated under reduced pressure. The residue was triturated with EtOAc (500 mL) for 30 min then filtered and the filtrate was concentrated under reduced pressure to afford 2,6-dichloro-5-fluoronicotinamide (95.2 g, 96% yield over two steps) as white solid. LCMS (ESI) m/z: [M+H] calcd for C₆H₄Cl₂FN₂O: 208.96; found 208.9. ¹H NMR (400 MHz, DMSO-d₆) δ 8.24 (d, J=7.9 Hz, 1H), 8.11 (br s, 1H), 7.95 (br s, 1H).

### Step 3: Synthesis of 2,6-dichloro-5-fluoro-N-((2-isopropyl-4-methylpyridin-3-yl)carbamoyl) nicotinamide

To a solution of 2,6-dichloro-5-fluoronicotinamide (60 g, 287 mmol, 1 equiv) in THF (250 mL) was added (COCl)₂ (43.72 g, 344 mmol, 1.2 equiv) and the resulting mixture was heated to 65° C. After 1 h the reaction was cooled to 0° C. and 2-isopropyl-4-methylpyridin-3-amine (43.1 g, 287 mmol, 1 equiv) was added. After 1 h the reaction was quenched with 1:1 sat. aq. NaCl/sat. aq. NH₄Cl (200 mL), extracted into EtOAc (3×300 mL), dried over Na₂SO₄, filtered, and concentrated under reduced pressure to afford 2,6-dichloro-5-fluoro-N-((2-isopropyl-4-methylpyridin-3-yl)carbamoyl) nicotinamide as a white solid, which was used without further purification. LCMS (ESI) m/z: [M+H] calcd for C₁₆H₁₆Cl₂FN₄O₂: 385.06; found 385.0.

### Step 4: Synthesis of 7-choro-8-fluoro-1-(2-isopropyl-4-methylpyridin-3-yl)pyrido [2,3-d]pyrimidine-2,4(1H,3H)-dione

To a solution of 2,6-dichloro-5-fluoro-N-((2-isopropyl-4-methylpyridin-3-yl)carbamoyl) nicotinamide (115 g, 298 mmol, 1 equiv) in THF (550 mL) at 0° C. was added KHMDS (1 M, 627 mL, 2.1 equiv) dropwise, and the resulting mixture was warmed to room temperature. After 2 h the reaction was quenched with sat. aq. NH₄Cl (500 mL) and extracted into EtOAc (3×400 mL). The combined organic phase was dried over Na₂SO₄, filtered, and concentrated under reduced pressure. The residue was purified by silica gel column chromatography (0→100% EtOAc/petroleum ether) to afford 7-chloro-8-fluoro-1-(2-isopropyl-4-methylpyridin-3-yl)pyrido [2,3-d]pyrimidine-2,4(1H,3H)-dione (70 g, 67% yield) as a white solid. LCMS (ESI) m/z: [M+H] calcd for C₁₆H₁₅ClFN₄O₂: 349.08; found 349.1. ¹H NMR (400 MHz, DMSO-d₆) δ 12.28 (br s, 1H), 8.63-8.39 (m, 2H), 7.28 (d, J=4.9 Hz, 1H), 2.86 (quin, J=6.6 Hz, 1H), 2.03 (s, 3H), 1.07 (d, J=6.8 Hz, 3H), 1.00 (d, J=6.6 Hz, 3H).

### Step 5: Synthesis of 6-fluoro-7-(2-fluorophenyl)-1-(2-isopropyl-4-methylpyridin-3-yl)pyrido [2,3-d]pyrimidine-2,4(1H,3H)-dione

To a solution of 7-chloro-8-fluoro-1-(2-isopropyl-4-methylpyridin-3-yl)pyrido [2,3-d]pyrimidine-2,4(1H,3H)-dione (2.0 g, 5.73 mmol, 1 equiv), (2-fluorophenyl)boronic acid (2.41 g, 17.2 mmol, 3 equiv) and KOAc (2.81 g, 28.7 mmol, 5 equiv) in dioxane (20 mL) and H₂O (4 mL) was added Pd(dppf)Cl₂ (420 mg, 573 μmol, 0.1 equiv) and the resulting mixture was heated to 90° C. for 2 h. The reaction was quenched with sat. aq. NaHCO₃ (15 mL) and extracted into EtOAc (3×20 mL). The combined organic phase was dried over Na₂SO₄, filtered, and concentrated under reduced pressure. The residue was purified by silica gel column chromatography (0→100% EtOAc/petroleum ether) to afford 6-fluoro-7-(2-fluorophenyl)-1-(2-isopropyl-4-methylpyridin-3-yl)pyrido [2,3-d]pyrimidine-2,4(1H,3H)-dione (2.2 g, 93% yield) as a yellow solid. LCMS (ESI) m/z: [M+H] calcd for C₂₂H₁₉F₂N₄O₂: 409.14; found 409.1.

### Step 6: Synthesis of 4-chloro-8-fluoro-7-(2-fluorophenyl)-1-(2-isopropyl-4-methylpyridin-3-yl) pyrido[2,3-d]pyrimidin-2(1H)-one

To a solution of 6-fluoro-7-(2-fluorophenyl)-1-(2-isopropyl-4-methylpyridin-3-yl)pyrido [2,3-d]pyrimidine-2,4(1H,3H)-dione (2.15 g, 5.26 mmol, 1 equiv) and N,N-diisopropylethylamine (3.06 g, 23.7 mmol, 4.5 equiv) in MeCN (20 mL) was added POCl₃ (3.23 g, 21.1 mmol, 4 equiv). The resulting mixture was heated to 80° C. for 1 h then concentrated under reduced pressure to afford 4-chloro-8-fluoro-7-(2-fluorophenyl)-1-(2-isopropyl-4-methylpyridin-3-yl) pyrido[2,3-d]pyrimidin-2(1H)-one as a brown oil which was used with further purification.

### Step 7: Synthesis of tert-butyl (1-(6-fluoro-7-(2-fluorophenyl)-1-(2-isopropyl-4-methylpyridin-3-yl)-2-oxo-1,2-dihydropyrido[2,3-d]pyrimidin-4-yl)piperidin-4-yl)carbamate

To a solution of 4-chloro-8-fluoro-7-(2-fluorophenyl)-1-(2-isopropyl-4-methylpyridin-3-yl) pyrido[2,3-d]pyrimidin-2(1H)-one (4.18 g, 9.79 mmol, 1 equiv) in MeCN at 0° C. was added N,N-diisopropylethylamine (6.33 g, 49.0 mmol, 5 equiv) followed by tert-butyl piperidin-4-ylcarbamate (2.35 g, 11.7 mmol, 1.2 equiv). The resulting mixture was warmed to room temperature. After 1 h the reaction was quenched with sat. aq. NH₄Cl (20 mL) and extracted into EtOAc (3×30 mL). The combined organic phase was dried over dried over Na₂SO₄, filtered and concentrated under reduced pressure. The residue was purified by silica gel column chromatography (0→100% EtOAc/petroleum ether) followed by reverse phase chromatography to afford tert-butyl (1-(6-fluoro-7-(2-fluorophenyl)-1-(2-isopropyl-4-methylpyridin-3-yl)-2-oxo-1,2-dihydropyrido[2,3-d]pyrimidin-4-yl)piperidin-4-yl)carbamate (2.05 g, 35% yield over two steps) as a white solid. LCMS (ESI) m/z: [M+H] calcd for C₃₂H₃₇F₂N₆O₃: 591.28; found 591.4. ¹H NMR (400 MHz, DMSO-d₆) δ 8.42 (d, J=4.9 Hz, 1H), 8.28 (d, J=9.7 Hz, 1H), 7.60-7.49 (m, 1H), 7.38-7.17 (m, 4H), 7.01 (br d, J=7.1 Hz, 1H), 4.38 (d, J=13.4 Hz, 2H), 3.66 (d, J=3.3 Hz, 1H), 3.43 (t, J=12.0 Hz, 2H), 2.71 (quin, J=6.7 Hz, 1H), 1.98-1.87 (m, 5H), 1.72-1.52 (m, 2H), 1.41 (s, 9H), 1.07 (d, J=6.7 Hz, 3H), 0.95 (d, J=6.7 Hz, 3H).

### Step 8: Synthesis of 4-(4-aminopiperidin-1-yl)-8-fluoro-7-(2-fluorophenyl)-1-(2-isopropyl-4-methylpyridin-3-yl)pyrido[2,3-d]pyrimidin-2(1H)-one

To a solution of tert-butyl (1-(6-fluoro-7-(2-fluorophenyl)-1-(2-isopropyl-4-methylpyridin-3-yl)-2-oxo-1,2-dihydropyrido[2,3-d]pyrimidin-4-yl)piperidin-4-ylcarbamate (310 mg, 524 μmol, 1 equiv) in DCM (3 mL) was added TFA (4.19 mmol, 321 μL, 8 equiv). The reaction was stirred for 18 h and then concentrated under reduced pressure to afford 4-(4-aminopiperidin-1-yl)-8-fluoro-7-(2-fluorophenyl)-1-(2-isopropyl-4-methylpyridin-3-yl)pyrido[2,3-d]pyrimidin-2(1H)-one as the TFA salt, which was used without further purification. LCMS (ESI) m/z: [M+H] calcd for C₂₈H₃₀F₂N₅O: 491.24; found 491.3.

### Step 9: Synthesis of 1-(1-(6-fluoro-7-(2-fluorophenyl)-1-(2-isopropyl-8-methylphenyl)-2-oxo-1,2-dihydropyrido[2,3-d]pyrimidin-4-yl)piperidin-4-yl)-3-propylthiourea

To a suspension of 4-(4-aminopiperidin-1-yl)-8-fluoro-7-(2-fluorophenyl)-1-(2-isopropyl-4-methylpyridin-3-yl)pyrido[2,3-d]pyrimidin-2(1H)-one TFA salt (71.5 mg, 121 μmol, 1 equiv) in DCM (1.00 mL) was added NEt₃ (50.5 μL, 363 μmol, 3 equiv) followed by 1-propyl isothiocyanate (12.4 μL, 121 μmol, 1 equiv). The resulting mixture was stirred for 17 h then diluted with DCM (20 mL), washed with H₂O (10 mL) then sat. aq. NaCl (10 mL), dried over Na₂SO₄, filtered, and concentrated under reduced pressure to afford 1-(1-(6-fluoro-7-(2-fluorophenyl)-1-(2-isopropyl-4-methylpyridin-3-yl)-2-oxo-1,2-dihydropyrido[2,3-d]pyrimidin-4-yl)piperidin-4-yl)-3-propylthiourea (56.2 mg, 78.4% yield) as a brown solid. LCMS (ESI) m/z: [M+H] calcd for C₃₂H₃₇F₂N₆OS: 592.27; found 592.5.

### Step 10: Synthesis of 6-fluoro-7-(2-fluorophenyl)-1-(2-isopropyl-4-methylpyridin-3-yl)-4-(4-(((propylimino)methylene)amino)piperidin-1-yl)pyrido[2,3-d]pyrimidin-2(1H)-one

To a solution of 1-(1-(6-fluoro-7-(2-fluorophenyl)-1-(2-isopropyl-4-methylpyridin-3-yl)-2-oxo-1,2-dihydropyrido[2,3-d]pyrimidin-4-yl)piperidin-4-yl)-3-propylthiourea (56.2 mg, 94.9 μmol, 1 equiv) in DCM (1.0 mL) was added N,N-diisopropylethylamine (49.4 μL, 284 μmol, 3 equiv) followed by 2-chloro-1-methylpyridin-1-ium iodide (36.2 mg, 142 μmol, 1.5 equiv). The resulting mixture was stirred for 16 h then filtered to remove solids and concentrated under reduced pressure. The crude residue was purified by reverse phase chromatography (40→100% MeCN/H₂O, 0.4% NH₄OH) to afford 6-fluoro-7-(2-fluorophenyl)-1-(2-isopropyl-4-methylpyridin-3-yl)-4-(4-(((propylimino)methylene)amino)piperidin-1-yl)pyrido[2,3-d]pyrimidin-2(1H)-one (6.1 mg, 11.5% yield) as a pale brown oil. LCMS (ESI) m/z: [M+H] calcd for C₃₁H₃₄F₂N₇O: 558.28; found 558.3; ¹H NMR (500 MHz, DMSO-d₆) δ 8.43 (d, J=4.9 Hz, 1H), 8.35 (d, J=9.5 Hz, 1H), 7.59-7.51 (m, 1H), 7.38-7.33 (m, 1H), 7.33-7.23 (m, 2H), 7.22 (dd, J=4.8, 0.8 Hz, 1H), 4.26 (dt, J=13.6, 4.3 Hz, 2H), 3.73 (tt, J=8.9, 4.0 Hz, 1H), 3.57 (ddd, J=13.3, 9.9, 2.9 Hz, 2H), 3.20 (t, J=6.8 Hz, 2H), 2.72 (p, J=6.7 Hz, 1H), 2.11-2.04 (m, 2H), 1.94 (s, 3H), 1.78-1.66 (m, 2H), 1.57 (h, J=7.2 Hz, 3H), 1.37 (d, J=9.1 Hz, 1H), 1.31-1.22 (m, 4H), 1.08 (d, J=6.7 Hz, 3H), 0.98-0.89 (m, 6H), 0.89-0.83 (m, 1H).

### Example 54—Synthesis of 6-fluoro-7-(2-fluorophenyl)-1-(2-isopropyl-4-methylpyridin-3-yl)-4-(4-((((2-methoxyethyl)imino)methylene)amino)piperidin-1-yl)pyrido[2,3-d]pyrimidin-2(1H)-one

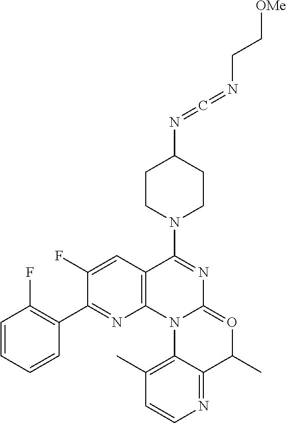

Synthesized according to the method of example 53, using 2-methoxyethyl isothiocyanate in place of 1-propyl isothiocyanate in step 9. LCMS (ESI) m/z: [M+H] calcd for C₃₁H₃₄F₂N₇O₂: 574.27; found 574.3.

### Example 55—Synthesis of 6-fluoro-7-(2-fluorophenyl)-1-(2-isopropyl-4-methylpyridin-3-yl)-4-(methyl(3-(((propylimino)methylene)amino)propyl)amino)pyrido[2,3-d]pyrimidin-2(1H)-one

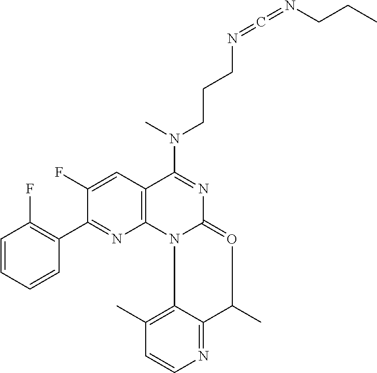

Synthesized according to the method of example 53, using tert-butyl (3-(methylamino)propyl)carbamate in place of tert-butyl piperidin-4-ylcarbamate in step 7. LCMS (ESI) m/z: [M+H] calcd for C₃₀H₃₄F₂N₇O: 546.28; found 546.3.

### Example 56—Synthesis of 6-fluoro-7-(2-fluorophenyl)-1-(2-isopropyl-4-methylpyridin-3-yl)-4-((3-((((2-methoxyethyl)imino)methylene)amino)propyl)methyl)amino)pyrido[2,3-d]pyrimidin-2(1H)-one

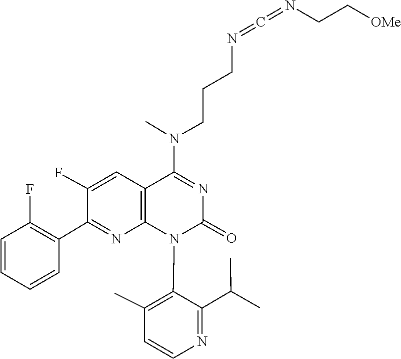 [SMILES: COCCN=C=NCCCN(C)c1nc(=O)n(-c2c(C)ccnc2C(C)C)c2nc(-c3ccccc3F)c(F)cc12]

Synthesized according to the method of example 53, using tert-butyl (3-(methylamino)propyl)carbamate in place of tert-butyl piperidin-4-ylcarbamate in step 7, and 2-methoxyethyl isothiocyanate in place of 1-propyl isothiocyanate in step 9. LCMS (ESI) m/z: [M+H] calcd for C₃₀H₃₄F₂N₇O₂: 562.27; found 562.3.

### Example 57—Synthesis of 4-((3-(((benzylimino)methylene)amino)propyl)methyl)amino)-6-fluoro-7-(2-fluorophenyl)-1-(2-isopropyl-4-methylpyridin-3-yl)pyrido[2,3-d]pyrimidin-2(1H)-one

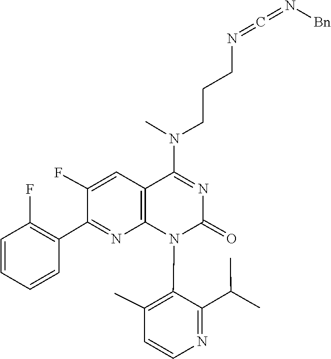 [SMILES: Cc1ccnc(C(C)C)c1-n1c(=O)nc(N(C)CCCN=C=NCc2ccccc2)c2cc(F)c(-c3ccccc3F)nc21]

Synthesized according to the method of example 53, using tert-butyl (3-(methylamino)propyl)carbamate in place of tert-butyl piperidin-4-ylcarbamate in step 7, and benzyl isothiocyanate in place of 2-methoxyethyl isothiocyanate in step 9. LCMS (ESI) m/z: [M+H] calcd for C₇₄H₃₄F₂N₇O: 594.28; found 594.3.

### Example 58—Synthesis of 6-fluoro-7-(2-fluorophenyl)-1-(2-isopropyl-4-methylpyridin-3-yl)-4-((3-((((4-methoxybenzyl)imino)methylene)amino)propyl)methyl)amino)pyrido[2,3-d]pyrimidin-2(1H)-one

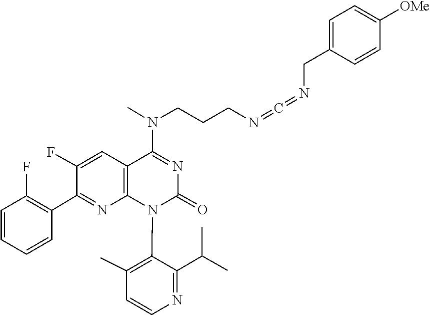 [SMILES: COc1ccc(CN=C=NCCCN(C)c2nc(=O)n(-c3c(C)ccnc3C(C)C)c3nc(-c4ccccc4F)c(F)cc23)cc1]

Synthesized according to the method of example 53, using tert-butyl (3-(methylamino)propyl)carbamate in place of tert-butyl piperidin-4-ylcarbamate in step 7, and 4-methoxybenzyl isothiocyanate in place of 2-methoxyethyl isothiocyanate in step 9. LCMS (ESI) m/z: [M+H] calcd for C₃₅H₃₆F₂N₇O₂: 624.29; found 624.3.

### Example 59—Synthesis of 4-((3-((((4-chlorobenzyl)imino)methylene)amino)propyl)methyl)amino)-6-fluoro-7-(2-fluorophenyl)-1-(2-isopropyl-4-methylpyridin-3-yl)pyrido[2,3-d]pyrimidin-2(1H)-one

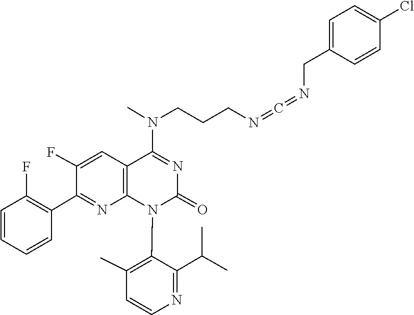 [SMILES: Cc1ccnc(C(C)C)c1-n1c(=O)nc(N(C)CCCN=C=NCc2ccc(Cl)cc2)c2cc(F)c(-c3ccccc3F)nc21]

Synthesized according to the method of example 53, using tert-butyl (3-(methylamino)propy)carbamate in place of tert-butyl piperidin-4-ylcarbamate in step 7, and 4-chlorobenzyl isothiocyanate in place of 2-methoxyethyl isothiocyanate in step 9. LCMS (ESI) m/z: [M+H] calcd for C₃₄H₃₃ClF₂N₇O: 628.24; found 628.2.

### Example 60—Synthesis of N-benzyl-N-(2-(7-(8-methylnaphthalen-1-yl)-2-(((S)-1-methylpyrrolidin-2-yl)methoxy)-5,6,7,8-tetrahydropyrido[3,4-d]pyrimidin-4-yl)-2-azaspiro[3.3]heptan-6-yl)methanediimine

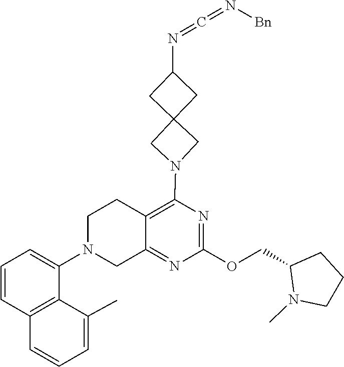 [SMILES: Cc1cccc2cccc(N3CCc4c(nc(OC[C@@H]5CCCN5C)nc4N4CC5(CC(N=C=NCc6ccccc6)C5)C4)C3)c12]

Synthesized according to the method of example 20, using tert-butyl (2-azaspiro[3.3]heptan-8-yl)carbamate in place of tert-butyl piperidin-4-ylcarbamate in step 10, and benzyl isothiocyanate in place of 2-methoxyethyl isothiocyanate in step 12. LCMS (ESI) m/z: [M+H] calcd for C₃₈H₄₄N₇O: 614.36; found 614.3.

### Example 61—Synthesis of N-(2-(7-(8-methylnaphthalen-1-yl)-2-(((S)-1-methylpyrrolidin-2-yl)methoxy)-5,6,7,8-tetrahydropyrido[3,4-d]pyrimidin-4-yl)-2-azaspiro[3.3]heptan-6-yl)-N-propylmethanediimine

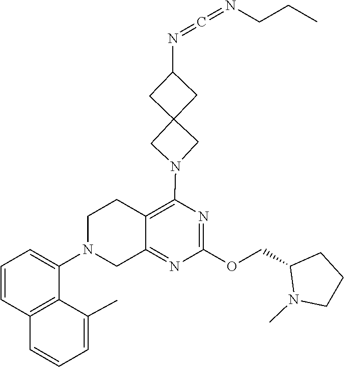 [SMILES: CCCN=C=NC1CC2(C1)CN(c1nc(OC[C@@H]3CCCN3C)nc3c1CCN(c1cccc4cccc(C)c14)C3)C2]

Synthesized according to the method of example 20, using tert-butyl (2-azaspiro[3.3]heptan-8-yl)carbamate in place of tert-butyl piperidin-4-ylcarbamate in step 10, and 1-propyl isothiocyanate in place of 2-methoxyethyl isothiocyanate in step 12. LCMS (ESI) m/z: [M+H] calcd for C₃₄H₄₄N₇O: 566.36; found 566.4.

### Example 62—Synthesis of N-(2-methoxyethyl)-N-(2-(7-(8-methylnaphthalen-1-yl)-2-(((S)-1-methylpyrrolidin-2-yl)methoxy)-5,6,7,8-tetrahydropyrido[3,4-d]pyrimidin-4-yl)-2-azaspiro[3.3]heptan-6-yl)methanediimine

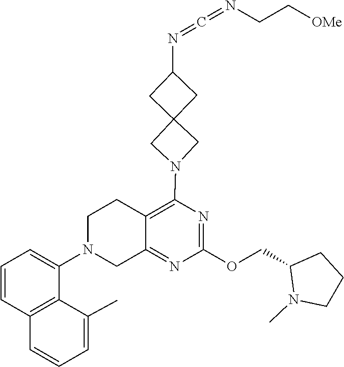 [SMILES: COCCN=C=NC1CC2(C1)CN(c1nc(OC[C@@H]3CCCN3C)nc3c1CCN(c1cccc4cccc(C)c14)C3)C2]

Synthesized according to the method of example 20, using tert-butyl (2-azaspiro[3.3]heptan-8-yl)carbamate in place of tert-butyl piperidin-4-ylcarbamate in step 10. LCMS (ESI) m/z: [M+H] calcd for C₃₄H₄₄N₇O₂: 582.36; found 582.4.

### Example 63—Synthesis of 2-((S)-1-((R)-aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

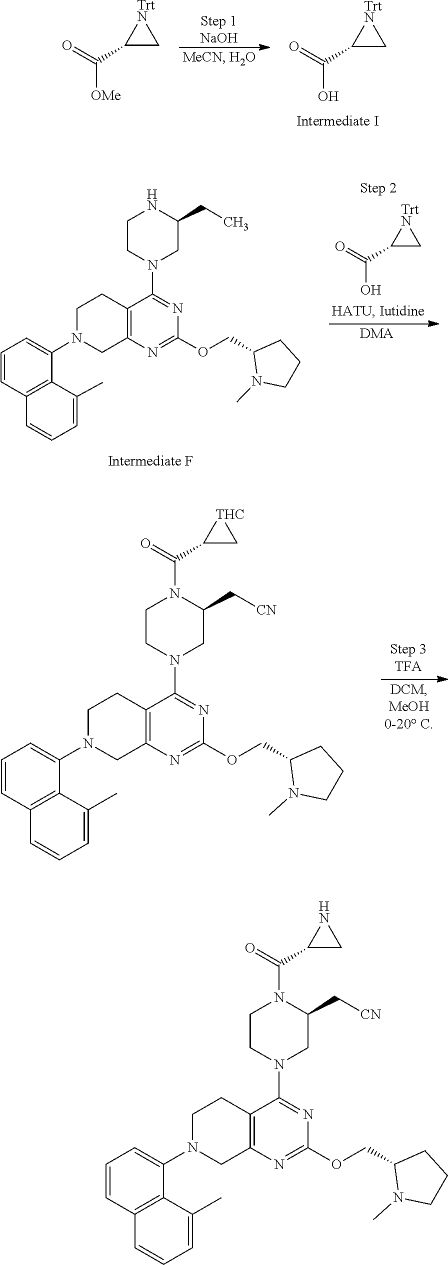

### Step 1: Synthesis of (R)-1-tritylaziridine-2-carboxylic Acid

Synthesized according to the method of example 16, step 1, using methyl (R)-1-tritylaziridine-2-carboxylate in place of methyl 1-tritylaziridine-2-carboxylate.

### Step 2: Synthesis of 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)-1-((R)-1-tritylaziridine-2-carbonyl)piperazin-2-yl)acetonitrile

To a suspension of (R)-1-tritylaziridine-2-carboxylic acid (178 mg, 0.51 mmol, 1.5 equiv) and HATU (193 mg, 0.51 mmol, 1.5 equiv) in DMA (3.4 mL) was added 2-((S)-4-(7-(8-methylnaphthalen-1-yl)-2-(((S)-1-methylpyrrolidin-2-yl)(methoxy)-5,6,7,8-tetrahydropyrido[3,4-d]pyrimidin-4-yl)piperazin-2-yl)acetonitrile (174 mg, 0.34 mmol, 1 equiv). The resulting mixture was stirred for 1 h then diluted with H₂O (10 mL) and DCM (10 mL). The aqueous layer was extracted into DCM (10 mL), and the combined organic phase was washed with sat. aq. NaHCO₃, dried over Na₂SO₄, filtered, and concentrated under reduced pressure. The crude product was taken on without further purification. LCMS (ESI) m/z: [M+Na] calcd for C₅₂H₅₄N₈O₂Na: 845.43; found 845.7.

### Step 3: Synthesis of 2-((S)-1-((R)-aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)-1-((R)-1-tritylaziridine-2-carbonyl)piperazin-2-yl)acetonitrile (279 mg, 0.34 mmol, 1 equiv) in DCM (1.7 mL) and MeOH (0.85 mL) at 0° C. was added TFA (0.20 mL, 2.7 mmol, 8 equiv). The resulting mixture was stirred for 10 min then warmed to room temperature. After 40 min the reaction was concentrated under reduced pressure and the resulting crude product was purified by reverse phase chromatography (20→100% MeCN/H₂O, 0.4% NH₄OH) to afford 2-((S)-1-((R)-aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (43.9 mg, 22%) as a white solid. LCMS (ESI) m/z: [M+H] calcd for C₃₃H₄₁N₈O₂: 581.34; found 581.3. ¹H NMR (500 MHz, Methanol-d₄) δ 7.70 (d, J=8.0 Hz, 1H), 7.65 (dd, J=7.9, 3.5 Hz, 1H), 7.42 (q, J=7.3 Hz, 1H), 7.36-7.27 (m, 2H), 7.25 (d, J=7.0 Hz, 1H), 4.60-4.48 (m, 1H), 4.41-4.03 (m, 6H), 3.82-3.64 (m, 2H), 3.59-3.35 (m, 3H), 3.29-3.01 (m, 6H), 2.92 (s, 3H), 2.90-2.81 (m, 1H), 2.82-2.61 (m, 2H), 2.50 (d, J=3.8 Hz, 3H), 2.35 (qd, J=9.1, 4.1 Hz, 1H), 2.09 (dq, J=12.8, 8.3 Hz, 1H), 1.98-1.78 (m, 4H), 1.71 (tt, J=13.0, 7.2 Hz, 1H).

### Example 64—Synthesis of 2-((S)-1-((S)-aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

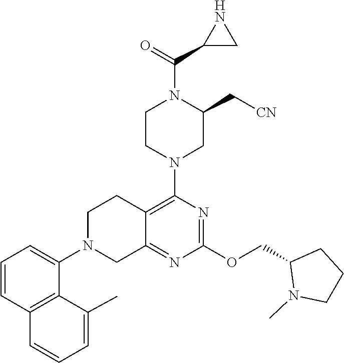 [SMILES: Cc1cccc2cccc(N3CCc4c(nc(OC[C@@H]5CCCN5C)nc4N4CCN(C(=O)[C@@H]5CN5)[C@@H](CC#N)C4)C3)c12]

Synthesized according to the method of example 63, using methyl (S)-1-tritylaziridine-2-carboxylate in place of methyl (R)-1-tritylaziridine-2-carboxylate in step 1. LCMS (ESI) m/z: [M+H] calcd for C₃₃H₄₁N₈O₂: 581.34; found 581.3.

### Example 65—Synthesis of 4-((S)-4-((R)-aziridine-2-carbonyl)-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one

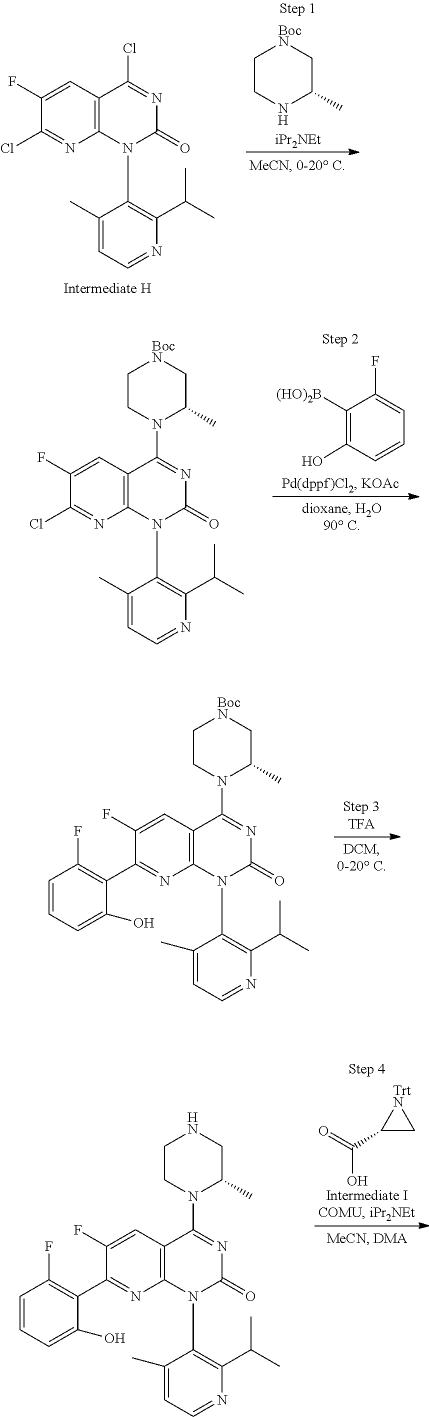

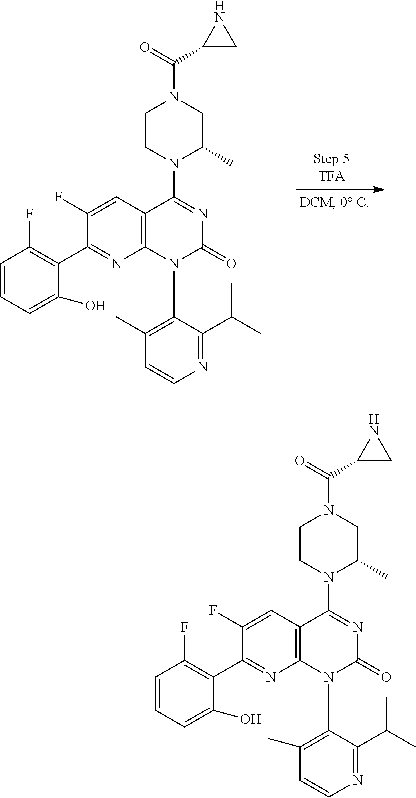 [SMILES: Cc1ccnc(C(C)C)c1-n1c(=O)nc(N2CCN(C(=O)[C@H]3CN3)C[C@@H]2C)c2cc(F)c(-c3c(O)cccc3F)nc21 ; Cc1ccnc(C(C)C)c1-n1c(=O)nc(N2CCN(C(=O)[C@H]3CN3)C[C@@H]2C)c2cc(F)c(-c3c(O)cccc3F)nc21]

### Step 1: Synthesis of (S)-tert-butyl 4-(7-chloro-6-fluoro-1-(2-isopropyl-4-methylpyridin-3-yl)-2-oxo-1,2-dihydropyrido[2,3-d]pyrimidin-4-yl)-3-methylpiperazine-1-carboxylate

To a solution of 4,7-dichloro-8-fluoro-1-(2-isopropyl-4-methylpyridin-3-yl) pyrido[2,3-d] pyrimidin-2(1H)-one (6.9 g crude, 17.2 mmol, 1 equiv) in MeCN (25 mL) at 0° C. was added N,N-diisopropylethylamine (11.1 g, 86.0 mmol, 5 equiv) followed by (S)-tert-butyl 3-methylpiperazine-1-carboxylate (4.35 g, 20.6 mmol, 1.2 equiv). The resulting mixture was warmed to room temperature, stirred for 1 h, then quenched with sat. aq. NaHCO₃ (20 mL) and extracted into EtOAc (3×20 mL). The combined organic phase was dried over dried over Na₂SO₄, filtered and concentrated under reduced pressure. The resulting crude product was purified by silica gel column chromatography (0→66% EtOAc/petroleum ether) to afford (S)-tert-butyl 4-(7-chloro-8-fluoro-1-(2-isopropyl-4-methylpyridin-3-yl)-2-oxo-1,2-dihydropyrido[2,3-d]pyrimidin-4-yl)-3-methylpiperazine-1-carboxylate (3.4 g, 37% yield) as a brown solid. ¹H NMR (400 MHz, DMSO-d₆) δ 8.48 (d, J=4.9 Hz, 1H), 8.37 (dd, J=5.4, 8.5 Hz, 1H), 7.25 (d, J=4.9 Hz, 1H), 4.83 (s, 1H), 4.24-4.09 (m, 1H), 3.82 (d, J=13.2 Hz, 1H), 3.73-3.56 (m, 2H), 3.18-2.88 (m, 2H), 2.71-2.56 (m, 1H), 1.93 (d, J=2.2 Hz, 3H), 1.45 (s, 9H), 1.31 (t, J=6.0 Hz, 3H), 1.08-1.02 (m, 3H), 1.00 (dd, J=2.8, 6.7 Hz, 3H).

### Step 2: Synthesis of (3S)-tert-butyl 4-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-3-methylpiperazine-1-carboxylate

To a solution of (S)-tert-butyl 4-(7-chloro-8-fluoro-1-(2-isopropyl-4-methylpyridin-3-yl)-2-oxo-1,2-dihydropyrido[2,3-d]pyrimidin-4-yl)-3-methylpiperazine-1-carboxylate (5 g, 9.42 mmol, 1 equiv), (2-fluoro-8-hydroxyphenyl)boronic acid (2.94 g, 18.8 mmol, 2 equiv) and KOAc (4.62 g, 47.1 mmol, 5 equiv) in dioxane (20 mL) and H₂O (4 mL) was added Pd(dppf)Cl₂ (689 mg, 0.942 mmol, 0.1 equiv). The resulting mixture was heated to 90° C. for 2 h then cooled to room temperature. The reaction was quenched with sat. aq. NaHCO₃ (20 mL), extracted into EtOAc (4×20 mL), and the combined organic phase was dried over Na₂SO₄, filtered and concentrated under reduced pressure. The resulting crude product was purified by silica gel column chromatography (0→66% EtOAc/petroleum ether) to afford (3S)-tert-butyl 4-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-3-methylpiperazine-1-carboxylate (2.9 g, 51% yield) as a yellow solid. LCMS (ESI) m/z: [M+H] calcd for C₃₂H₃₇F₂N₆O₄: 607.28; found 607.2. ¹H NMR (400 MHz, DMSO-d₆) δ 10.21 (br d, J=1.2 Hz, 1H), 8.38 (d, J=4.9 Hz, 1H), 8.27 (dd, J=9.2, 12.6 Hz, 1H), 7.32-7.22 (m, 1H), 7.18 (d, J=5.0 Hz, 1H), 6.77-6.64 (m, 2H), 5.02-4.75 (m, 1H), 4.24 (t, J=14.1 Hz, 1H), 3.84 (d, J=12.7 Hz, 2H), 3.75-3.56 (m, 1H), 3.26-2.87 (m, 2H), 2.79-2.63 (m, 1H), 1.93-1.86 (m, 3H), 1.45 (s, 9H), 1.35 (dd, J=6.7, 10.7 Hz, 3H), 1.07 (dd, J=1.7, 6.7 Hz, 3H), 0.93 (dd, J=2.1, 6.7 Hz, 3H).

### Step 3: Synthesis of 6-fluoro-7-(2-fluoro-6-hydroxyphenyl)-1-(2-isopropyl-4-methylpyridin-3-yl)-4-((S)-2-methylpiperazin-1-yl)pyrido[2,3-d]pyrimidin-2(1H)-one

To a solution of (3S)-tert-butyl 4-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-3-methylpiperazine-1-carboxylate (1.8 g, 2.97 mmol, 1 equiv) in DCM (18 mL) at 0° C. was added TFA (6.77 g, 59.3 mmol, 20 equiv). The resulting mixture was warmed to room temperature, stirred for 2 h then concentrated under reduced pressure. The residue was dissolved in MeCN (2 mL) then added dropwise to MTBE (20 mL). The mixture was stirred for 20 min, filtered and the solid cake was dried under reduced pressure to afford 6-fluoro-7-(2-fluoro-8-hydroxyphenyl)-1-(2-isopropyl-4-methylpyridin-3-yl)-4-((S)-2-methylpiperazin-1-yl)pyrido[2,3-d]pyrimidin-2(1H)-one trifluoroacetate (1.78 g, 93% yield) as a pale yellow solid. LCMS (ESI) m/z: [M+H] calcd for C₂₇H₂₉F₂N₆O₂: 507.22; found 507.3. ¹H NMR (400 MHz, Methanol-d₄) δ 8.61-8.54 (m, 1H), 8.27 (dd, J=8.7, 12.8 Hz, 1H), 7.66 (br d, J=5.5 Hz, 1H), 7.31-7.20 (m, 1H), 6.70-6.56 (m, 2H), 5.29-5.09 (m, 1H), 4.68-4.53 (m, 1H), 4.07-3.87 (m, 1H), 3.59-3.40 (m, 4H), 3.16-2.97 (m, 1H), 2.20 (d, J=14.9 Hz, 3H), 1.69 (dd, J=7.1, 12.0 Hz, 3H), 1.30 (dd, J=4.7, 6.8 Hz, 3H), 1.14 (t, J=6.5 Hz, 3H).

### Step 4: Synthesis of 6-fluoro-7-(2-fluoro-6-hydroxyphenyl)-1-(2-isopropyl-4-methylpyridin-3-yl)-4-((S)-2-methyl-4-((R)-1-tritylaziridine-2-carbonyl)piperazin-1-yl)pyrido[2,3-d]pyrimidin-2(1H)-one

To a suspension of (R)-1-tritylaziridine-2-carboxylic acid (48.7 mg, 148 μmol, 1.5 equiv) in DMA (0.18 mL) was added a solution of 6-fluoro-7-(2-fluoro-6-hydroxyphenyl)-1-(2-isopropyl-4-methylpyridin-3-yl)-4-((S)-2-methylpiperazin-1-yl)pyrido[2,3-d]pyrimidin-2(1H)-one trifluoroacetate (61.3 mg, 98.7 μmol, 1 equiv) in MeCN (1 mL), followed by N,N-diisopropylethylamine (51.4 μL, 296 μmol, 3 equiv) and COMU (59.1 mg, 138 μmol, 1.4 equiv). The resulting mixture was stirred for 1 h 30 min then diluted with DCM (20 mL), washed with 5% aq. citric acid (10 mL) then sat. aq. NaHCO₃ (10 mL), dried over Na₂SO₄, filtered, and concentrated under reduced pressure. The resulting crude product was used without further purification. LCMS (ESI) m/z: [M+H] calcd for C₄₉H₄₆F₂N₇O₃: 818.36; found 818.3.

### Step 5: Synthesis of 4-((S)-4-((R)-aziridine-2-carbonyl)-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one

To a solution of 6-fluoro-7-(2-fluoro-6-hydroxyphenyl)-1-(2-isopropyl-4-methylpyridin-3-yl)-4-((S)-2-methyl-4-((R)-1-tritylaziridine-2-carbonyl)piperazin-1-yl)pyrido[2,3-d]pyrimidin-2(1H)-one (80 mg, 97.8 μmol, 1 equiv) in DCM (2 mL) at 0° C. was added TFA (0.5 mL, 6.52 mmol, 67 equiv). The resulting mixture was stirred for 1 h then concentrated under reduced pressure. The resulting crude product was purified by reverse phase chromatography (10→100% MeCN/H₂O, 0.4% NH₄OH) to afford 6-fluoro-7-(2-fluoro-6-hydroxyphenyl)-1-(2-isopropyl-4-methylpyridin-3-yl)-4-((S)-2-methyl-4-((R)-1-tritylaziridine-2-carbonyl)piperazin-1-yl)pyrido[2,3-d]pyrimidin-2(1H)-one (23.7 mg, 42% yield) as a white solid. LCMS (ESI) m/z: [M+H] calcd for C₃₀H₃₂F₂N₇O: 576.25; found 576.2. ¹H NMR (500 MHz, Methanol-d₄) δ 8.42 (d, J=5.0 Hz, 1H), 8.31-8.22 (m, 1H), 7.29-7.21 (m, 2H), 6.67 (d, J=8.3 Hz, 1H), 6.60 (t, J=8.9 Hz, 1H), 5.26-4.98 (m, 1H), 4.65-4.28 (m, 3H), 4.21 (s, 1H), 4.07-3.56 (m, 2H), 3.50-3.37 (m, 1H), 3.30-3.16 (m, 1H), 3.12-2.90 (m, 1H), 2.85 (dq, J=12.7, 6.7 Hz, 1H), 2.05 (dd, J=9.3, 4.2 Hz, 3H), 1.98-1.79 (m, 2H), 1.53 (dt, J=20.1, 6.1 Hz, 3H), 1.22 (d, J=6.4 Hz, 3H), 1.05 (dd, J=6.6, 2.6 Hz, 3H).

### Example 66—Synthesis of 4-((S)-4-((S)-aziridine-2-carbonyl)-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one

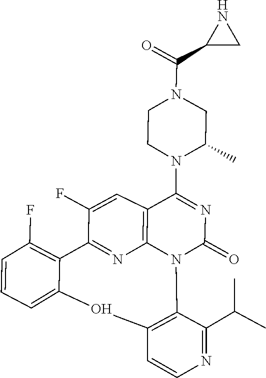 [SMILES: CC(C)c1nccc2c1-n1c(=O)nc(N3CCN(C(=O)[C@@H]4CN4)C[C@@H]3C)c3cc(F)c(nc31)-c1c(F)cccc1O2]

Synthesized according to the method of example 65, using (S)-1-tritylaziridine-2-carboxylic acid in place of (R)-1-tritylaziridine-2-carboxylic acid in step 4. LCMS (ESI) m/z: [M+H] calcd for C₃₀H₃₂F₂N₇O: 576.25; found 576.2.

### Example 67—Synthesis of 6-fluoro-7-(2-fluoro-6-hydroxyphenyl)-1-(2-isopropyl-4-methylpyridin-3-yl)-4-(4-(2,2,2-trifluoroacetyl)piperidin-1-yl)pyrido[2,3-d]pyrimidin-2(1H)-one

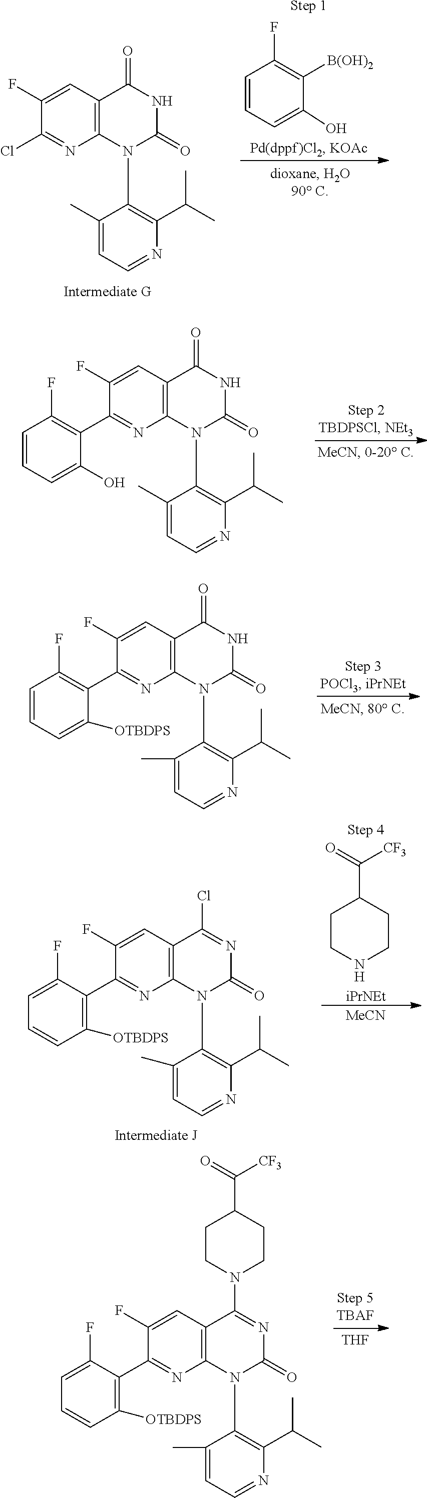

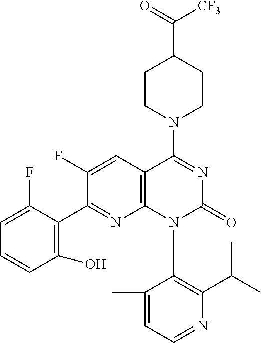 [SMILES: Cc1ccnc(C(C)C)c1-n1c(=O)nc(N2CCC(C(=O)C(F)(F)F)CC2)c2cc(F)c(-c3c(O)cccc3F)nc21]

### Step 1: Synthesis of 6-fluoro-7-(2-fluoro-6-hydroxyphenyl)-1-(2-isopropyl-4-methylpyridin-3-yl)pyrido[2,3-d]pyrimidine-2,4(1H,3H)-dione

To a solution of 7-chloro-6-fluoro-1-(2-isopropyl-4-methylpyridin-3-yl)pyrido[2,3-d]pyrimidine-2,4(1H,3H)-dione (3 g, 8.60 mmol, 1 equiv), (2-fluoro-6-hydroxyphenyl)boronic acid (2.68 g, 17.2 mmol, 2 equiv) and KOAc (4.22 g, 43.0 mmol, 5 equiv) in dioxane (15 mL) and H₂O (3 mL) was added Pd(dppf)Cl₂ (629 mg, 0.86 mmol, 0.1 equiv). The resulting mixture was heated to 90° C. for 2 h, cooled to room temperature and quenched with sat. aq. NaHCO₃ (15 mL), then extracted into EtOAc (3×20 mL). The combined organic phase was dried over Na₂SO₄, filtered and concentrated under reduced pressure. The resulting crude residue was purified by silica gel column chromatography (0→100% EtOAc/petroleum ether) to afford 6-fluoro-7-(2-fluoro-6-hydroxyphenyl)-1-(2-isopropyl-4-methylpyridin-3-yl)pyrido[2,3-d]pyrimidine-2,4(1H,3H)-dione (2.7 g, 74% yield) as a yellow solid.

### Step 2: 7-(2-((tert-butyldiphenylsilyl)oxy)-6-fluorophenyl)-6-fluoro-1-(2-isopropyl-4-methylpyridin-3-yl)pyrido[2,3-d]pyrimidine-2,4(1H,3H)-dione

To a solution of 6-fluoro-7-(2-fluoro-6-hydroxyphenyl)-1-(2-isopropyl-4-methylpyridin-3-yl) pyrido[2,3-d]pyrimidine-2,4(1H,3H)-dione (2.4 g, 5.66 mmol, 1 equiv) and NEt₃ (2.29 g, 22.6 mmol, 4 equiv) in MeCN (24 mL) at 0° C. was added TBDPSCl (1.87 g, 6.79 mmol, 1.2 equiv). The resulting mixture was warmed to room temperature, stirred for 1 h, quenched with sat. aq. NaHCO₃ (20 mL), then extracted into EtOAc (3×30 mL). The combined organic phase was dried over Na₂SO₄, filtered and concentrated under reduced pressure. The resulting crude residue was purified by silica gel column chromatography (0→50% EtOAc/petroleum ether) to afford 7-(2-((tert-butyldiphenylsilyl)oxy)-6-fluorophenyl)-6-fluoro-1-(2-isopropyl-4-methylpyridin-3-yl)pyrido[2,3-d]pyrimidine-2,4(1H,3H)-dione (2.4 g, 64% yield) as a white solid. LCMS (ESI) m/z: [M+H] calcd for C₃₈H₃₇F₂N₄O₃Si: 663.25; found 663.3. ¹H NMR (400 MHz, DMSO-d₆) δ 12.29 (s, 1H), 8.58-8.43 (m, 2H), 7.72-7.65 (m, 1H), 7.64-7.58 (m, 1H), 7.55-7.39 (m, 5H), 7.39-7.22 (m, 4H), 7.16-7.04 (m, 1H), 6.81 (dt, J=3.9, 8.7 Hz, 1H), 6.14 (dd, J=8.4, 16.3 Hz, 1H), 2.96 (td, J=6.5, 13.3 Hz, 1H), 2.81 (quin, J=6.7 Hz, 1H), 2.08 (s, 1H), 1.81 (s, 2H), 1.10 (t, J=6.2 Hz, 3H), 1.04 (d, J=6.6 Hz, 2H), 0.82 (d, J=6.7 Hz, 1H), 0.72 (d, J=12.5 Hz, 9H).

### Step 3: Synthesis of 7-(2-((tert-butyldiphenylsilyl)oxy)-6-fluorophenyl)-4-chloro-6-fluoro-1-(2-isopropyl-4-methylpyridin-3-yl)pyrido[2,3-d]pyrimidin-2(1H)-one

To a solution of 7-(2-((tert-butyldiphenylsilyl)oxy)-6-fluorophenyl)-6-fluoro-1-(2-isopropyl-4-methylpyridin-3-yl)pyrido[2,3-d]pyrimidine-2,4(1H,3H)-dione (0.25 g, 0.377 mmol, 1 equiv) and N,N-diisopropylethylamine (439 mg, 3.39 mmol, 9 equiv) in MeCN (5 mL) was added POCl₃ (463 mg, 3.02 mmol, 8 equiv). The resulting mixture was heated to 80° C. for 1 h then concentrated under reduced pressure to afford 7-(2-((tert-butyldiphenylsilyl)oxy)-6-fluorophenyl)-4-chloro-6-fluoro-1-(2-isopropyl-4-methylpyridin-3-yl)pyrido[2,3-d]pyrimidin-2(1H)-one as a brown oil, which was used without further purification.

### Step 4: Synthesis of 7-(2-((tert-butyldiphenylsilyl)oxy)-6-fluorophenyl)-6-fluoro-1-(2-isopropyl-4-methylpyridin-3-yl)-4-(4-(2,2,2-trifluoroacetyl)piperidin-1-yl)pyrido[2,3-d]pyrimidin-2(1H)-one

To a solution of 7-(2-((tert-butyldiphenylsiyl)oxy)-6-fluorophenyl)-4-chloro-6-fluoro-1-(2-isopropyl-4-methylpyridin-3-yl)pyrido[2,3-d]pyrimidin-2(1H)-one (0.25 g, 0.367 mmol, 1 equiv) in MeCN (5 mL) at 0° C. was added N,N-diisopropylethylamine (237 mg, 1.83 mmol, 5 equiv) followed by 2,2,2-trifluoro-1-(piperidin-4-yl)ethenone hydrochloride (66.5 mg, 0.305 mmol, 0.83 equiv). The resulting mixture was warmed to room temperature, stirred for 30 min, then quenched with sat. aq. NH₄Cl (20 mL) and extracted into EtOAc (3×20 mL). The combined organic phase was dried over dried over Na₂SO₄, filtered and concentrated under reduced pressure. The resulting crude product was purified by silica gel column chromatography (15-50% EtOAc/petroleum ether) to afford 7-(2-((tert-butyldiphenylsilyl)oxy)-6-fluorophenyl)-6-fluoro-1-(2-isopropyl-4-methylpyridin-3-yl)-4-(4-(2,2,2-trifluoroacetyl)piperidin-1-yl)pyrido[2,3-d]pyrimidin-2(1H)-one (0.2 g, 66% yield over 2 steps) as yellow solid. ¹H NMR (400 MHz, DMSO-d₆) δ 8.51-8.38 (m, 2H), 7.71-7.58 (m, 2H), 7.55-7.20 (m, 9H), 7.17-7.06 (m, 1H), 6.90-6.73 (m, 2H), 6.14 (dd, J=8.6, 15.9 Hz, 1H), 4.62-4.39 (m, 2H), 2.76-2.69 (m, 1H), 2.18-2.06 (m, 1H), 1.92 (s, 3H), 1.74 (s, 2H), 1.08 (dd, J=2.8, 6.7 Hz, 3H), 1.01 (d, J=6.5 Hz, 2H), 0.84 (d, J=6.7 Hz, 1H), 0.71 (d, J=6.0 Hz, 9H).

### Step 5: Synthesis of 6-fluoro-7-(2-fluoro-6-hydroxyphenyl)-1-(2-isopropyl-4-methylpyridin-3-yl)-4-(4-(2,2,2-trifluoroacetyl)piperidin-1-yl)pyrido[2,3-d]pyrimidin-2(1H)-one

To a suspension of 7-(2-((tert-butyldiphenylsiyl)oxy)-6-fluorophenyl)-6-fluoro-1-(2-isopropyl-4-methylpyridin-3-yl)-4-(4-(2,2,2-trifluoroacetyl)piperidin-1-yl)pyrido[2,3-d]pyrimidin-2(1H)-one (0.16 g, 0.194 mmol, 1 equiv) in THF (2 mL) at 0° C. was added TBAF (1 M, 0.387 mL, 0.387 mmol, 2 equiv). The resulting mixture was stirred for 10 min then extracted into EtOAc (3×20 mL). The combined organic phase was washed with sat. aq. NaCl, dried over Na₂SO₄, filtered and concentrated under reduced pressure. The crude residue was purified by reverse phase chromatography (30→55% MeCN/H₂O, 10 mM NH₄HCO₃) to afford 6-fluoro-7-(2-fluoro-6-hydroxyphenyl)-1-(2-isopropyl-4-methylpyridin-3-yl)-4-(4-(2,2,2-trifluoroacetyl)piperidin-1-yl)pyrido[2,3-d]pyrimidin-2(1H)-one (70 mg, 61% yield) as yellow solid. LCMS (ESI) m/z: [M+H] calcd for C₂₉H₂₇F₅N₅O₃: 588.20; found 588.2. ¹H NMR (400 MHz, Methanol-d₄) δ 8.43-8.33 (m, 1H), 8.27-8.18 (m, 1H), 7.33-7.18 (m, 2H), 6.75-6.47 (m, 2H), 4.81-4.64 (m, 2H), 3.54-3.32 (m, 2H), 2.94-2.72 (m, 1H), 2.30-2.14 (m, 1H), 2.13-2.06 (m, 1H), 2.05-1.96 (m, 3H), 1.91-1.68 (m, 2H), 1.25-1.13 (m, 3H), 1.03 (d, J=6.8 Hz, 2H).

### Example 68—Synthesis of (1-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)piperidin-4-yl)boronic Acid

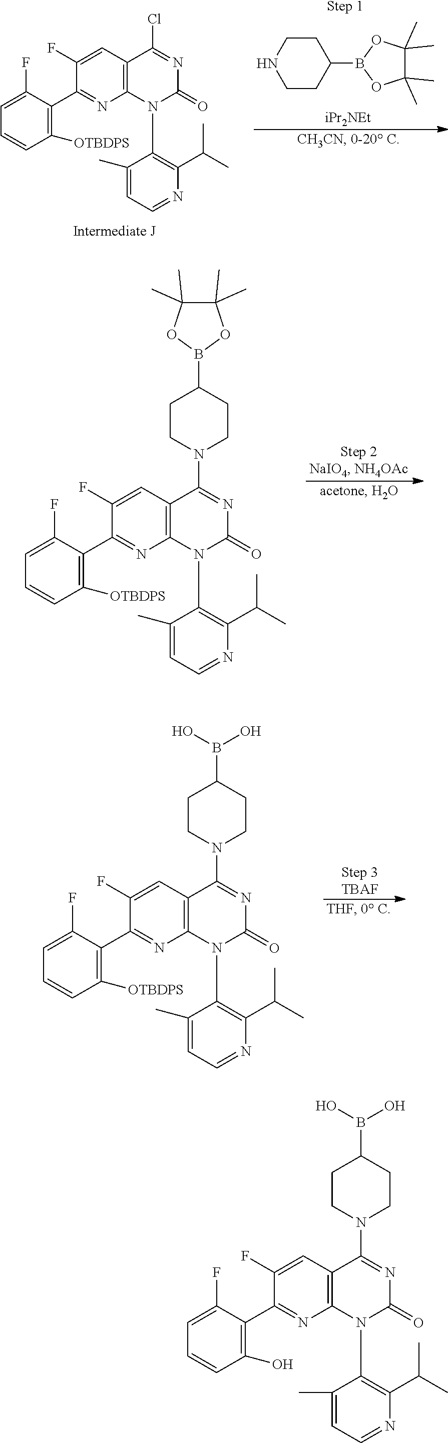

### Step 1: 7-(2-((tert-butyldiphenylsilyl)oxy)-6-fluorophenyl)-6-fluoro-1-(2-isopropyl-4-methylpyridin-3-yl)-4-(4-(4,4,5,5-tetramethyl-1,3,2-dioxaborolan-2-yl)piperidin-1-yl)pyrido[2,3-d]pyrimidin-2(1H)-one

To a solution of 7-(2-((tert-butyldiphenylsilyl)oxy)-6-fluorophenyl)-4-chloro-6-fluoro-1-(2-isopropyl-4-methylpyridin-3-yl)pyrido[2,3-d]pyrimidin-2(1H)-one (420 mg crude, 0.57 mmol, 1 equiv) in MeCN (4 mL) at 0° C. was added N,N-diisopropylethylamine (370 mg, 2.87 mmol, 5 equiv) followed by 4-(4,4,5,5-tetramethyl-1,3,2-dioxaborolan-2-yl)piperidine hydrochloride (170 mg, 0.69 mmol, 1.2 equiv). The resulting mixture was warmed to room temperature, stirred for 30 min, then quenched with sat. aq. NH₄Cl (4 mL) and extracted into EtOAc (3×4 mL). The combined organic phase was dried over Na₂SO₄, filtered and concentrated under reduced pressure. The resulting crude product was purified by silica gel column chromatography (0→50% EtOAc/petroleum ether) to afford 7-(2-((tert-butyldiphenylsilyl)oxy)-6-fluorophenyl-6-fluoro-1-(2-isopropyl-4-methylpyridin-3-yl)-4-(4-(4,4,5,5-tetramethyl-1,3,2-dioxaborolan-2-yl)piperidin-1-yl)pyrido[2,3-d]pyrimidin-2(1H)-one (410 mg, 83% yield) as a yellow solid. ¹H NMR (400 MHz, DMSO-d₆) δ 8.49-8.40 (m, 2H), 7.72-7.59 (m, 2H), 7.54-7.20 (m, 9H), 7.15-7.06 (m, 1H), 6.83 (t, J=8.7 Hz, 1H), 6.14 (dd, J=8.3, 16.2 Hz, 1H), 4.26-4.07 (m, 2H), 3.69-3.45 (m, 2H), 2.76-2.68 (m, 1H), 1.99 (s, 2H), 1.86-1.69 (m, 5H), 1.41-1.30 (m, 1H), 1.21 (d, J=3.1 Hz, 12H), 1.10-1.05 (m, 3H), 1.00 (d, J=6.8 Hz, 2H), 0.83 (d, J=6.8 Hz, 2H), 0.70 (d, J=5.7 Hz, 9H).

### Step 2: Synthesis of (1-(7-(2-((tert-butyldiphenylsilyl)oxy)-6-fluorophenyl)-6-fluoro-1-(2-isopropyl-4-methylpyridin-3-yl)-2-oxo-1,2-dihydropyrido[2,3-d]pyrimidin-4-yl)piperidin-4-yl)boronic Acid

To a solution of the 7-(2-((tert-butyldiphenylsilyl)oxy)-6-fluorophenyl)-6-fluoro-1-(2-isopropyl-4-methylpyridin-3-yl)-4-(4-(4,4,5,5-tetramethyl-1,3,2-dioxaborolan-2-yl)piperidin-1-yl)pyrido[2,3-d]pyrimidin-2(1)-one (400 mg, 0.467 mmol, 1 equiv) in acetone (4 mL) and H₂O (0.4 mL) was added NaIO₄ (300 mg, 1.40 mmol, 3 equiv) and NH₄OAc (1 M in H₂O, 4.00 mL, 8.56 equiv). The resulting mixture was stirred for 2 h then poured into H₂O (4 mL), extracted into EtOAc (2×4 mL), washed with sat. aq. NaCl (4 mL), dried over Na₂SO₄, filtered and concentrated under reduced pressure. The resulting crude product was purified by silica gel column chromatography (0→100% EtOAc/petroleum ether) to afford (1-(7-(2-((tert-butyldiphenylsilyl)oxy)-6-fluorophenyl-6-fluoro-1-(2-isopropyl-4-methylpyridin-3-yl)-2-oxo-1,2-dihydropyrido[2,3-d]pyrimidin-4-yl)piperidin-4-yl)boronic acid (270 mg, 75% yield) as a yellow solid.

¹H NMR (400 MHz, DMSO-d₆) δ 8.50-8.34 (m, 2H), 7.72-7.60 (m, 2H), 7.58 (br s, 2H), 7.53-7.20 (m, 9H), 7.10 (q, J=8.0 Hz, 1H), 6.83 (t, J=8.8 Hz, 1H), 6.14 (dd, J=8.3, 16.1 Hz, 1H), 4.47-4.21 (m, 2H), 3.58-3.36 (m, 2H), 2.80-2.70 (m, 1H), 1.93-1.88 (m, 3H), 1.86-1.68 (m, 4H), 1.08 (d, J=6.6 Hz, 3H), 1.01 (d, J=6.8 Hz, 2H), 0.84 (d, J=6.8 Hz, 2H), 0.70 (d, J=5.9 Hz, 9H).

### Step 3: Synthesis of (1-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)piperidin-4-yl)boronic Acid

To a solution of (1-(7-(2-((tert-butyldiphenylsilyl)oxy)-6-fluorophenyl)-6-fluoro-1-(2-isopropyl-4-methylpyridin-3-yl)-2-oxo-1,2-dihydropyrido[2,3-d]pyrimidin-4-yl)piperidin-4-yl)boronic acid (260 mg, 0.336 mmol, 1 equiv) in THF (2.5 mL) at 0° C. was added TBAF (1 M in THF, 0.67 mL, 2 equiv). The resulting mixture was stirred for 10 min then poured into H₂O (3 mL), extracted into EtOAc (3×3 mL), washed with sat. aq. NaCl (3 mL), dried over Na₂SO₄, filtered and concentrated under reduced pressure. The resulting crude product was purified by reverse phase chromatography (25→45% MeCN/H₂O, 10 mM NH₄HCO₃) to afford (1-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)piperidin-4-yl)boronic acid (110 mg, 60% yield) as a pale yellow solid. LCMS (ESI) m/z: [M+H] calcd for C₂₇H₂₉BF₂N₅O₄: 536.22; found 536.3. ¹H NMR (400 MHz, CDCl₃) δ 9.47 (br s, 1H), 8.62 (d, J=4.9 Hz, 1H), 8.00 (d, J=9.5 Hz, 1H), 7.35-7.28 (m, 1H), 7.22 (d, J=4.9 Hz, 1H), 6.77-6.62 (m, 2H), 6.35 (br s, 1H), 4.39 (d, J=13.3 Hz, 2H), 3.73-3.38 (m, 2H), 2.80 (td, J=6.6, 13.4 Hz, 1H), 2.14-2.00 (m, 5H), 1.98-1.85 (m, 2H), 1.48-1.35 (m, 1H), 1.31-1.21 (m, 3H), 1.08 (d, J=6.7 Hz, 3H).

### Example 69—Synthesis of 4-(4-((((benzylimino)methylene)amino)methyl)piperidin-1-yl)-6-fluoro-7-(2-fluorophenyl)-1-(2-isopropyl-4-methylpyridin-3-yl)pyrido[2,3-d]pyrimidin-2(1H)-one

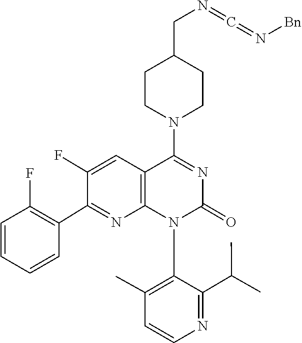 [SMILES: Cc1ccnc(C(C)C)c1-n1c(=O)nc(N2CCC(CN=C=NCc3ccccc3)CC2)c2cc(F)c(-c3ccccc3F)nc21]

Synthesized according to the method of example 53, using tert-butyl (piperidin-4-ylmethyl)carbamate in place of tert-butyl piperidin-4-ylcarbamate in step 7, and benzyl isothiocyanate in place of 1-propyl isothiocyanate in step 9. LCMS (ESI) m/z: [M+H] calcd for C₃₆H₃₆F₂N₇O: 620.29; found 620.3.

### Example 70—Synthesis of 6-fluoro-7-(2-fluorophenyl)-1-(2-isopropyl-4-methylpyridin-3-yl)-4-(4-(((((2-methoxyethyl)imino)methylene)amino)methyl)piperidin-1-yl)pyrido[2,3-d]pyrimidin-2(1H)-one

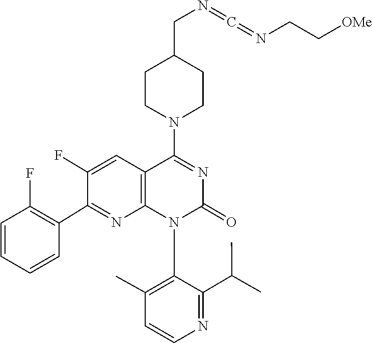 [SMILES: COCCN=C=NCC1CCN(c2nc(=O)n(-c3c(C)ccnc3C(C)C)c3nc(-c4ccccc4F)c(F)cc23)CC1]

Synthesized according to the method of example 53, using tert-butyl (piperidin-4-ylmethyl)carbamate in place of tert-butyl piperidin-4-ylcarbamate in step 7, and 2-methoxyethyl isothiocyanate in place of 1-propyl isothiocyanate in step 9. LCMS (ESI) m/z: [M+H] calcd for C₃₂H₃₆F₂N₇O₂: 588.29; found 588.3.

### Example 71—Synthesis of 6-fluoro-7-(2-fluorophenyl)-1-(2-isopropyl-4-methylpyridin-3-yl)-4-(4-((((propylimino)methylene)amino)methyl)piperidin-1-yl)pyrido[2,3-d]pyrimidin-2(1H)-one

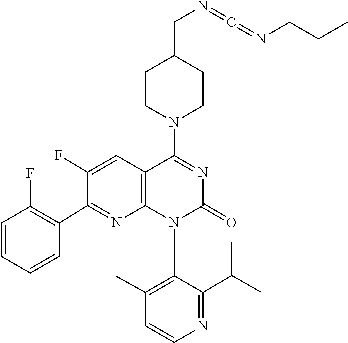 [SMILES: CCCN=C=NCC1CCN(c2nc(=O)n(-c3c(C)ccnc3C(C)C)c3nc(-c4ccccc4F)c(F)cc23)CC1]

Synthesized according to the method of example 53, using tert-butyl (piperidin-4-ylmethyl)carbamate in place of tert-butyl piperidin-4-ylcarbamate in step 7. LCMS (ESI) m/z: [M+H] calcd for C₃₂H₃₅F₂N₇O: 572.29; found 572.3.

### Example 72—Synthesis of N-((1r,3S)-3-((((2-methoxyethyl)imino)methylene)amino)cyclobutyl)-7-(8-methylnaphthalen-1-yl)-2-(((S)-1-methylpyrrolidin-2-yl)methoxy)-5,6,7,8-tetrahydropyrido[3,4-d]pyrimidin-4-amine

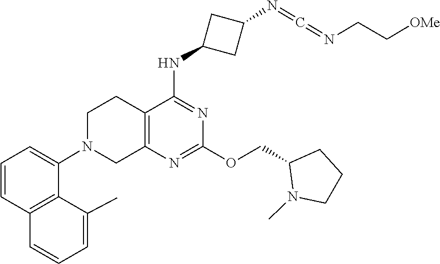 [SMILES: COCCN=C=N[C@H]1C[C@H](Nc2nc(OC[C@@H]3CCCN3C)nc3c2CCN(c2cccc4cccc(C)c24)C3)C1]

Synthesized according to the method of example 20, using tert-butyl ((1r,3r)-3-aminocyclobutyl)carbamate in place of tert-butyl piperidin-4-ylcarbamate in step 10. LCMS (ESI) m/z: [M+H] calcd for C₃₂H₄₂N₇O₂: 556.34; found 556.4.

### Example 73—Synthesis of N-(2-methoxyethyl)-N—((R)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)pyrrolidin-3-yl)methanediimine

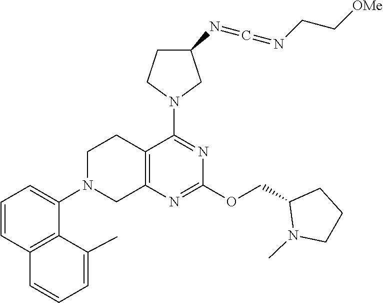 [SMILES: COCCN=C=N[C@@H]1CCN(c2nc(OC[C@@H]3CCCN3C)nc3c2CCN(c2cccc4cccc(C)c24)C3)C1]

Synthesized according to the method of example 20, using tert-butyl (R)-pyrrolidin-3-ylcarbamate in place of tert-butyl piperidin-4-ylcarbamate in step 10. LCMS (ESI) m/z: [M+H] calcd for C₃₂H₄₂N₇O₂: 556.34; found 556.4.

### Example 74—Synthesis of N-(2-methoxyethyl)-N—((S)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)pyrrolidin-3-yl)methanediimine

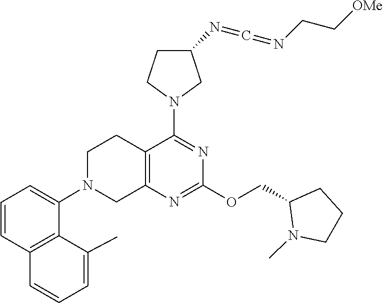 [SMILES: COCCN=C=N[C@H]1CCN(c2nc(OC[C@@H]3CCCN3C)nc3c2CCN(c2cccc4cccc(C)c24)C3)C1]

Synthesized according to the method of example 20, using tert-butyl (S)-pyrrolidin-3-ylcarbamate in place of tert-butyl piperidin-4-ylcarbamate in step 10. LCMS (ESI) m/z: [M+H] calcd for C₃₂H₄₂N₇O₂: 556.34; found 556.4.

### Example 75—Synthesis of 2-((3S,4R)-4-((((2-methoxyethyl)imino)methylene)amino)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile

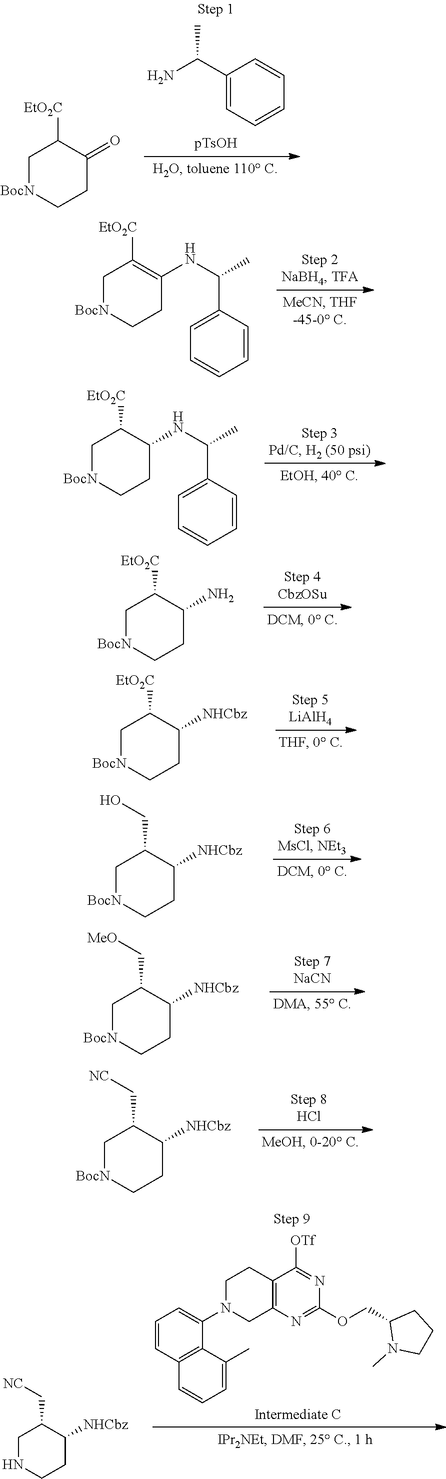

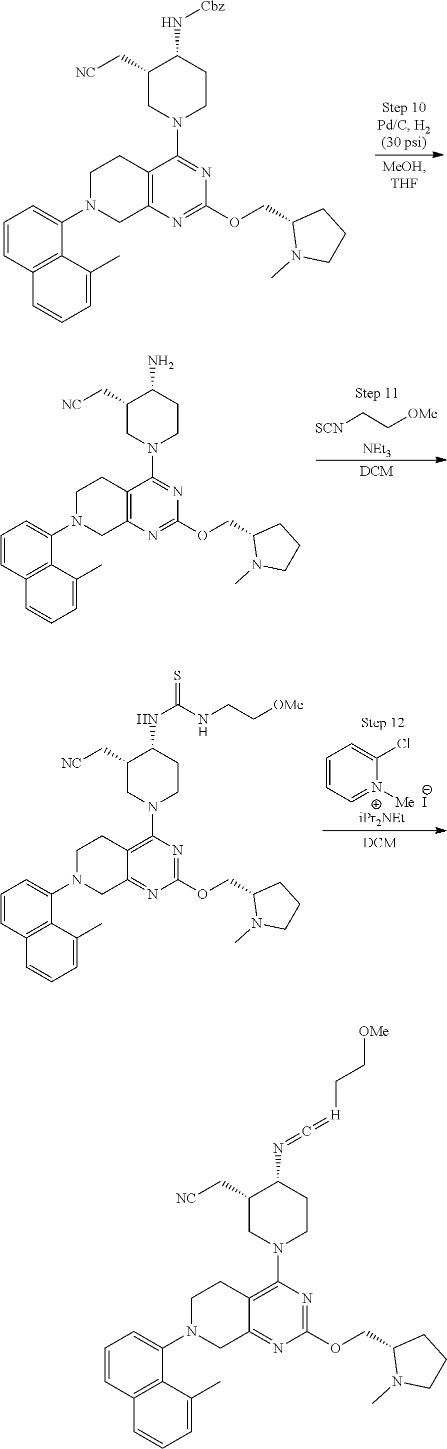

### Step 1: Synthesis of 1-(tert-butyl) 3-ethyl (R)-4-((1-phenylethyl)amino)-5,6-dihydropyridine-1,3(2H)-dicarboxylate

To a solution of 1-tert-butyl 3-ethyl 4-oxopiperidine-1,3-dicarboxylate (150 g, 553 mmol, 1 equiv) in toluene (2.25 L) was added (R)-1-phenylethanamine (77.05 g, 636 mmol, 1.15 equiv) and p-TsOH (10.47 g, 60.8 mmol, 0.11 equiv). The resulting mixture was heated to 140° C. with a Dean Stark trap for 18 h, then cooled to room temperature. The reaction was washed with sat. aq. NaHCO₃ (3×900 mL), and the organic phase was washed with sat. aq. NaCl (800 ml), dried over Na₂SO₄, filtered, and concentrated under reduced pressure. The crude residue was purified by silica gel column chromatography (0→10% EtOAc/petroleum ether) to afford 1-(tert-butyl) 3-ethyl (R)-4-((1-phenylethyl)amino)-5,6-dihydropyridine-1,3(2H)-dicarboxylate (200 g, 97% yield) as a yellow solid. ¹H NMR (400 MHz, CDCl₃) δ 9.25 (br d, J=7.3 Hz, 1H), 7.36-7.30 (m, 2H), 7.26-7.21 (m, 3H), 4.61 (m, J=6.9 Hz, 1H), 4.24-4.15 (m, 2H), 4.11-4.03 (m, 2H), 3.49-3.38 (m, 1H), 3.37-3.24 (m, 1H), 2.39 (d, J=17.1 Hz, 1H), 2.13-2.05 (m, 1H), 1.50 (d, J=6.7 Hz, 3H), 1.44 (s, 9H), 1.34-1.27 (m, 3H).

### Step 2: Synthesis of 1-(tert-butyl) 3-ethyl (3S,4R)-4-(((R)-1-phenylethyl)amino)piperidine-1,3-dicarboxylate

Two separate reactions were run in parallel. For each reaction, to a suspension of NaBH₄ (8.08 g, 214 mmol, 2 equiv) in THF (0.58 L) at 0° C. was added dropwise TFA (73.1 g, 641 mmol, 6 equiv). The resulting mixture was stirred for 10 min then cooled to −45° C. and a solution of 1-(tert-butyl) 3-ethyl (R)-4-((1-phenylethylamino)-5,6-dihydropyridine-1,3(2)-dicarboxylate (40 g, 107 mmol, 1 equiv) in MeCN (192 mL) was added. The reaction was stirred for 1 h, then warmed to 0° C. and stirred for 1 h. The two separate reaction mixtures were combined, adjusted to pH 7 with 25% aq. NH₄OH, and concentrated under reduced pressure. The resulting residue was poured into H₂O (500 mL), cooled to 10° C., and aq. NH₄OH (108 mL) was added. The mixture was extracted into EtOAc (3×200 mL) and the combined organic phase was dried over Na₂SO₄, filtered and concentrated under reduced pressure. The residue was purified by silica gel column chromatography (0→10% EtOAc/petroleum ether) followed by reverse phase chromatography (15→45% MeCN/H₂O, 0.1% formic acid). Fractions containing desired product were combined, cooled to 0° C., adjusted to pH 7 with sat. aq. NaHCO₃, and concentrated under reduced pressure to remove MeCN. The resulting mixture was extracted into EtOAc (3×600 mL), and the combined organic phase was washed with sat. aq. NaCl (3×400 mL), dried over Na₂SO₄, filtered, and concentrated under reduced pressure. The resulting colorless oil was dissolved in MTBE (55 mL), cooled to 0° C., and added HCl (1 M in dioxane, 85 mL, 0.8 equiv). The mixture was stirred for 30 min, then added heptane (200 mL), stirred for 1 h, and filtered. The filter cake was washed with heptane (3×30 mL), then triturated with heptane (100 mL) for 10 min. The mixture was filtered, and the cake was washed with heptane (3×30 mL) then dried under vacuum. The resulting solid was suspended in H₂O (200 mL), adjusted to pH 8 with sat. aq. NaHCO₃, and extracted into EtOAc (3×200 mL). The combined organic phase was washed with sat. aq. NaCl (3×100 mL), dried over Na₂SO₄, filtered and concentrated under reduced pressure to afford 1-(tert-butyl) 3-ethyl (3S,4R)-4-(((R)-1-phenylethyl)amino)piperidine-1,3-dicarboxylate (39.8 g, 49% yield) as a colorless oil. ¹H NMR (400 MHz, CDCl₃) δ 7.36-7.28 (m, 4H), 7.27-7.21 (m, 1H), 4.25-4.15 (m, 2H), 3.98 (d, J=9.2 Hz, 1H), 3.88 (q, J=6.5 Hz, 1H), 3.67 (s, 1H), 3.20 (dd, J=3.8, 13.8 Hz, 1H), 3.02 (ddd, J=3.7, 9.1, 13.2 Hz, 1H), 2.87 (td, J=4.0, 8.5 Hz, 1H), 2.82-2.71 (m, 1H), 1.81-1.70 (m, 2H), 1.43 (s, 9H), 1.35-1.27 (m, 6H).

### Step 3: Synthesis of 1-(tert-butyl) 3-ethyl (3S,4R)-4-aminopiperidine-1,3-dicarboxylate

To a solution of 1-(tert-butyl) 3-ethyl (3S,4R)-4-(((R)-1-phenylethyl)amino)piperidine-1,3-dicarboxylate (39 g, 104 mmol, 1 equiv) in EtOH (156 mL) was added Pd/C (13 g, 10% purity). The resulting mixture was stirred under H₂ (50 psi) at 40° C. for 24 h then filtered through celite, washed with EtOAc (3×200 mL), and concentrated under reduced pressure to afford 1-(tert-butyl) 3-ethyl (3S,4R)-4-aminopiperidine-1,3-dicarboxylate (28 g, 99% yield) as a colorless liquid. ¹H NMR (400 MHz, CDCl₃) δ 4.14-4.02 (m, 2H), 3.70-3.52 (m, 3H), 3.44-3.25 (m, 3H), 1.76-1.56 (m, 2H), 1.45-1.33 (m, 9H), 1.27-1.14 (m, 3H).

### Step 4: Synthesis of 1-(tert-butyl) 3-ethyl (3S,4R)-4-(((benzyloxy)carbonyl)amino)piperidine-1,3-dicarboxylate

To a solution of 1-(tert-butyl) 3-ethyl (3S,4R)-4-aminopiperidine-1,3-dicarboxylate (23 g, 84.4 mmol, 1 equiv) in THF (230 mL) at 0° C. was added benzyl (2,5-dioxopyrrolidin-1-yl) carbonate (21.05 g, 84.4 mmol, 1 equiv). The resulting mixture was stirred for 10 min then added dropwise to H₂O (900 mL) at 0° C. and extracted into DCM (3×200 mL). The combined organic phase was washed with sat. aq. NaCl (300 mL), dried over Na₂SO₄, filtered, and concentrated under reduced pressure. The residue was purified by silica gel column chromatography (10→25% EtOAc/petroleum ether) to afford 1-(tert-butyl) 3-ethyl (3S,4R)-4-(((benzyloxy)carbonyl)amino)piperidine-1,3-dicarboxylate (30 g, 73% yield) as a colorless liquid. ¹H NMR (400 MHz, CDCl₃) δ 7.41-7.29 (m, 5H), 5.11 (s, 2H), 4.93 (br s, 1H), 4.32-4.20 (m, 1H), 4.19-4.11 (m, 1H), 4.03 (br s, 1H), 3.68 (s, 1H), 3.66-3.58 (m, 1H), 3.37 (d, J=3.5 Hz, 1H), 3.34 (d, J=3.1 Hz, 1H), 3.32-3.22 (m, 1H), 2.98 (s, 3H), 2.39 (s, 1H), 1.74 (s, 1H), 1.46 (s, 9H).

### Step 5: Synthesis of tert-butyl (3S,4R)-4-(((benzyloxy)carbonylamino)-3-(hydroxymethyl)piperidine-1-carboxylate

To a solution of 1-(tert-butyl) 3-ethyl (3S,4R)-4-(((benzyloxy)carbonyl)amino)piperidine-1,3-dicarboxylate (32 g, 78.7 mmol, 1 equiv) in THF (320 mL) at 0° C. was added LiAlH₄ (4.48 g, 118 mmol, 1.5 equiv) over 20 min. The resulting mixture was stirred for 2 h then quenched with dropwise addition of H₂O (4.48 mL), filtered, and the filter cake was washed with EtOAc (3×100 mL). The filtrate was poured into H₂O (300 mL) and the aqueous phase was extracted into EtOAc (3×200 mL). The combined organic phase was washed with sat. aq. NaCl (200 mL), dried over Na₂SO₄, filtered, and concentrated under reduced pressure. The residue was purified by silica gel column chromatography (10→50% EtOAc/petroleum ether) to afford tert-butyl (3S,4R)-4-(((benzyloxy)carbonyl)amino)-3-(hydroxymethylpiperidine-1-carboxylate (16.5 g, 57% yield) as a colorless oil. ¹H NMR (400 MHz, CDCl₃) δ 7.40-7.31 (m, 5H), 5.21 (s, 1H), 5.12 (s, 2H), 4.17-4.04 (m, 1H), 3.73-3.37 (m, 3H), 3.34-2.97 (m, 3H), 2.15-1.98 (m, 1H), 1.70 (s, 1H), 1.60 (s, 1H), 1.46 (s, 9H).

### Step 6: Synthesis of tert-butyl (3S,4R)-4-(((benzyloxy)carbonylamino)-3-(((methylsulfonyloxy)methylpiperidine-1-carboxylate

To a solution of tert-butyl (3S,4R)-4-(((benzyloxy)carbonyl)amino)-3-(hydroxymethyl)piperidine-1-carboxylate (16 g, 43.9 mmol, 1 equiv) in DCM (160 mL) at 0° C. was added NEt₃ (6.66 g, 65.9 mmol, 1.5 equiv) followed by MsCl (5.48 g, 47.8 mmol, 1.09 equiv). The resulting mixture was warmed to room temperature and stirred for 1 h, then added dropwise to H₂O (180 mL) at 0° C. and extracted into DCM (3×80 mL). The combined organic phase was washed with sat. aq. NaCl (200 mL), dried over Na₂SO₄, filtered, and concentrated under reduced pressure to afford tert-butyl (3S,4R)-4-(((benzyloxy)carbonyl)amino)-3-(((methylsulfonyl)oxy)methyl)piperidine-1-carboxylate which was used without further purification.

### Step 7: Synthesis of tert-butyl (3S,4R)-4-(((benzyloxy)carbonylamino)-3-(cyanomethyl)piperidine-1-carboxylate

To a solution of tert-butyl (3S,4R)-4-(((benzyloxy)carbonyl)amino)-3-(((methylsulfonyloxy)methylpiperidine-1-carboxylate (19 g, 42.9 mmol, 1 equiv) in DMA (380 mL) was added NaCN (4.21 g, 85.9 mmol, 2 equiv) and the resulting mixture was heated to 55° C. for 18 h then cooled to room temperature, poured into H₂O (1 L), and extracted into EtOAc (3×300 mL). The combined organic phase was washed with sat. aq. NaCl (3×200 mL), dried over Na₂SO₄, filtered, and concentrated under reduced pressure. The crude residue was purified by silica gel column chromatography (10→100% EtOAc/petroleum ether) to afford tert-butyl (3S,4R)-4-(((benzyloxy)carbonyl)amino)-3-(cyanomethyl)piperidine-1-carboxylate (10.5 g, 58% yield over 2 steps) as a colorless solid. ¹H NMR (400 MHz, CDCl₃) δ 7.43-7.32 (m, 5H), 5.23-5.01 (m, 2H), 4.76 (br s, 1H), 4.13-3.65 (m, 3H), 3.60-3.01 (m, 2H), 2.51-2.36 (m, 2H), 2.34-2.20 (m, 1H), 1.71 (dt, J=4.8, 8.8 Hz, 2H), 1.53-1.42 (m, 9H).

### Step 8: Synthesis of benzyl ((3S,4R)-3-(cyanomethyl)piperidin-4-yl)carbamate

Three separate reactions were run in parallel. For each reaction, to a solution of tert-butyl (3S,4R)-4-(((benzyloxy)carbonyl)amino)-3-(cyanomethyl)piperidine-1-carboxylate (3 g, 8.03 mmol, 1 equiv) in MeOH (24 mL) at 0° C. was added HCl (4M in MeOH, 45 mL, 22.4 equiv) and the resulting mixture was stirred for 30 min. The three separate reaction mixtures were combined and concentrated under reduced pressure to afford benzyl ((3S,4R)-3-(cyanomethyl)piperidin-4-yl)carbamate hydrochloride as a white solid, which was used without further purification. ¹H NMR (400 MHz, Methanol-d₄) δ 7.51-7.24 (m, 5H), 5.12 (s, 2H), 4.18 (d, J=3.2 Hz, 1H), 3.35 (s, 1H), 3.27 (t, J=3.9 Hz, 1H), 3.23-3.05 (m, 2H), 2.67-2.58 (m, 1H), 2.57-2.47 (m, 2H), 2.10-1.90 (m, 2H).

### Step 9: Synthesis of benzyl ((3S,4R)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)carbamate

Two separate reactions were run in parallel. For each reaction, to a solution of (S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate (7.95 g, 7.41 mmol, 1 equiv) and N,N-diisopropylethylamine (9.57 g, 74.1 mmol, 10 equiv) in DMF (37.5 mL) was added benzyl ((3S,4R)-3-(cyanomethyl)piperidin-4-yl)carbamate hydrochloride (2.41 g, 7.78 mmol, 1.05 equiv) and the resulting mixture was stirred for 20 min. The two separate reaction mixtures were combined and added dropwise to H₂O (800 mL), then extracted into EtOAc (3×200 mL). The combined organic phase was washed with sat. aq. NaCl (3×150 mL), dried over Na₂SO₄, filtered and concentrated under reduced pressure. The residue was purified by silica gel column chromatography (0→10% MeOH/EtOAc) to afford benzyl ((3S,4R)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)carbamate (8.3 g, 78% yield over 2 steps) as a brown solid. ¹H NMR (400 MHz, CDCl₃) δ 7.68 (d, J=8.2 Hz, 1H), 7.63 (dd, J=3.2, 7.8 Hz, 1H), 7.43-7.36 (m, 5H), 7.35-7.30 (m, 2H), 7.25-7.19 (m, 2H), 5.23-5.05 (m, 2H), 4.95 (br s, 1H), 4.51-4.36 (m, 1H), 4.27-4.07 (m, 3H), 3.85 (d, J=18.1 Hz, 1H), 3.72-3.57 (m, 2H), 3.56-3.45 (m, 2H), 3.41-3.24 (m, 1H), 3.22-3.04 (m, 3H), 2.92 (d, J=4.0 Hz, 3H), 2.83-2.67 (m, 1H), 2.52 (d, J=7.9 Hz, 5H), 2.39-2.25 (m, 2H), 1.87-1.72 (m, 4H).

### Step 10: Synthesis of 2-((3S,4R)-4-amino-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile

Two separate reactions were run in parallel. For each reaction, to a solution of benzyl ((3S,4R)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)carbamate (4 g, 6.06 mmol, 1 equiv) in MeOH (100 mL) and THF (100 mL) was added Pd/C (4 g, 10% purity). The resulting mixture was stirred under H₂ (30 psi) for 0.5 h then Pd/C (2 g, 10% purity) was added to the mixture. After 0.5 h, the two separate reaction mixtures were combined, filtered through celite, washed with MeOH (3×300 mL) then THF (3×300 mL), and concentrated under reduced pressure. The crude residue was purified by reverse phase chromatography (5→35% MeCN/H₂O, 0.225% formic acid). Fractions containing 2-((3S,4R)-4-amino-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile were combined, adjusted to pH 7 with sat. aq. NaHCO₃, and concentrated under reduce pressure to remove MeCN. The resulting aqueous phase was extracted into EtOAc (3×200 mL) and the combined organic phase was washed with sat. aq. NaCl (3×100 mL), dried over Na₂SO₄, filtered and concentrated under reduced pressure to afford 2-((3S,4R)-4-amino-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile (2.6 g, 41% yield) as a white solid. LCMS (ESI) m/z: [M+H] calcd for C₃₁H₄₀N₇O: 526.32; found 526.4. ¹H NMR (400 MHz, Methanol-d₄) δ 7.68 (d, J=7.9 Hz, 1H), 7.66-7.61 (m, 1H), 7.40 (q, J=7.7 Hz, 1H), 7.34-7.26 (m, 2H), 7.26-7.20 (m, 1H), 4.42-4.28 (m, 2H), 4.15-4.01 (m, 1H), 3.93 (dd, J=5.7, 13.3 Hz, 1H), 3.83 (dd, J=7.3, 13.6 Hz, 1H), 3.71 (d, J=17.7 Hz, 1H), 3.67-3.56 (m, 2H), 3.55-3.44 (m, 1H), 3.29-3.19 (m, 2H), 3.18-2.99 (m, 3H), 2.91 (s, 3H), 2.73 (td, J=6.8, 13.6 Hz, 1H), 2.69-2.58 (m, 2H), 2.48 (d, J=2.0 Hz, 3H), 2.46-2.27 (m, 2H), 2.26-1.95 (m, 2H), 1.95-1.58 (m, 5H).

### Step 11: Synthesis of 1-((3S,4R)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-3-(2-methoxyethyl)thiourea

To a solution of 2-((3S,4R)-4-amino-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile (50 mg, 95.1 μmol, 1 equiv) in DCM (951 μL) was added NEt₃ (79.3 μL, 570 μmol, 6 equiv) followed by 2-methoxyethyl isothiocyanate (11.2 μL, 104 μmol, 1.1 equiv). The resulting mixture was stirred for 24 h then diluted with DCM (20 mL), washed with H₂O (10 mL) followed by sat. aq. NaCl (10 mL), dried over Na₂SO₄, filtered, and concentrated under reduced pressure to afford 1-((3S,4R)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-3-(2-methoxyethyl)thiourea as a pale brown oil which was used without further purification. LCMS (ESI) m/z: [M+H] calcd for C₃₅H₄₇N₈O₂S: 643.35; found 643.3.

### Step 12: Synthesis of 2-((3S,4R)-4-((((2-methoxyethyl)imino)methylene)amino)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile

To a solution of 1-((3S,4R)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-3-(2-methoxyethyl)thiourea (61.0 mg, 95 μmol, 1 equiv) in DCM (949 μL) was added N,N-diisopropylethylamine (49.5 μL, 285 μmol, 3 equiv) followed by 2-chloro-1-methylpyridin-1-ium iodide (36.2 mg, 142 μmol, 1.5 equiv). The resulting mixture was stirred for 24 h then filtered to remove solids and concentrated under reduced pressure. The crude residue was purified by reverse phase chromatography (40→100% MeCN/H₂O, 0.4% NH₄OH) to afford 2-((3S,4R)-4-((((2-methoxyethyl)imino)methylene)amino)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile (18.8 mg, 33% over 2 steps) as a pale brown solid. LCMS (ESI) m/z: [M+H] calcd for C₃₅H₄₅N₈O₂: 609.37; found 609.4. ¹H NMR (500 MHz, Methanol-d₄) δ 7.70 (d, J=8.1 Hz, 1H), 7.66 (ddd, J=8.2, 3.2, 1.3 Hz, 1H), 7.42 (q, J=7.5 Hz, 1H), 7.37-7.28 (m, 2H), 7.26 (d, J=7.0 Hz, 1H), 4.39 (ddd, J=11.2, 6.3, 2.1 Hz, 1H), 4.32 (ddd, J=11.0, 9.2, 5.4 Hz, 1H), 4.10 (dd, J=17.8, 14.1 Hz, 1H), 4.01 (dt, J=8.7, 3.8 Hz, 2H), 3.96 (d, J=13.3 Hz, 1H), 3.83 (d, J=13.9 Hz, 1H), 3.76-3.61 (m, 1H), 3.61-3.49 (m, 3H), 3.44-3.37 (m, 5H), 3.32-3.21 (m, 1H), 3.21-3.14 (m, 1H), 3.08 (dt, J=9.7, 4.7 Hz, 1H), 2.94 (s, 3H), 2.75 (p, J=7.0 Hz, 1H), 2.68-2.62 (m, 1H), 2.61-2.53 (m, 2H), 2.51 (d, J=2.0 Hz, 3H), 2.48-2.40 (m, 1H), 2.40-2.31 (m, 1H), 2.25-1.89 (m, 4H), 1.87-1.78 (m, 2H), 1.74 (td, J=12.3, 11.5, 5.4 Hz, 1H).

### Example 76—Synthesis of 2-((3S,4S)-4-((((2-methoxyethyl)imino)methylene)amino)-1-(7-(8-methylnaphthalen-1-yl)-2-(((S)-1-methylpyrrolidin-2-yl)methoxy)-5,6,7,8-tetrahydropyrido[3,4-d]pyrimidin-yl)piperidin-3-yl)acetonitrile

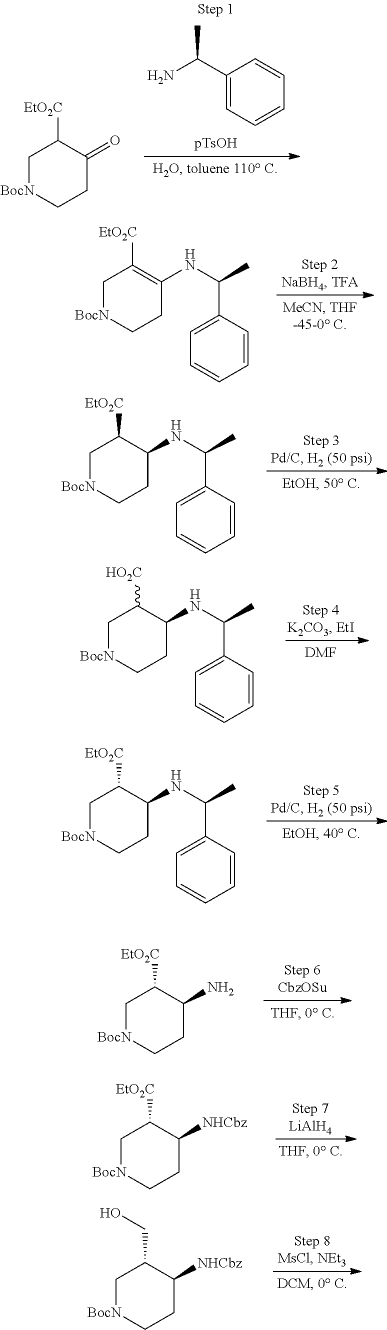

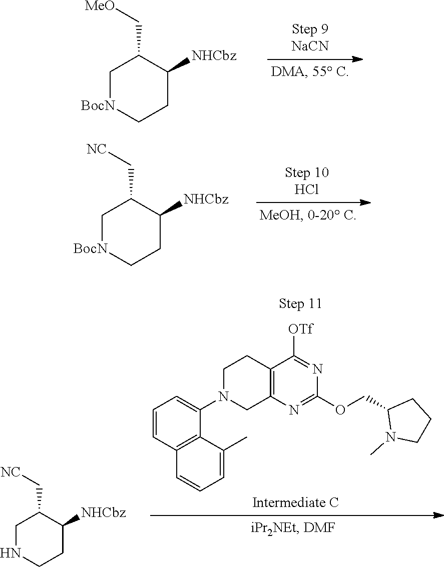 [SMILES: COC[C@H]1CN(C(=O)OC(C)(C)C)CC[C@@H]1NC(=O)OCc1ccccc1 ; CC(C)(C)OC(=O)N1CC[C@H](NC(=O)OCc2ccccc2)[C@@H](CC#N)C1 ; N#CC[C@H]1CNCC[C@@H]1NC(=O)OCc1ccccc1 ; Cc1cccc2cccc(N3CCc4c(nc(OC[C@@H]5CCCN5C)nc4OS(=O)(=O)C(F)(F)F)C3)c12]

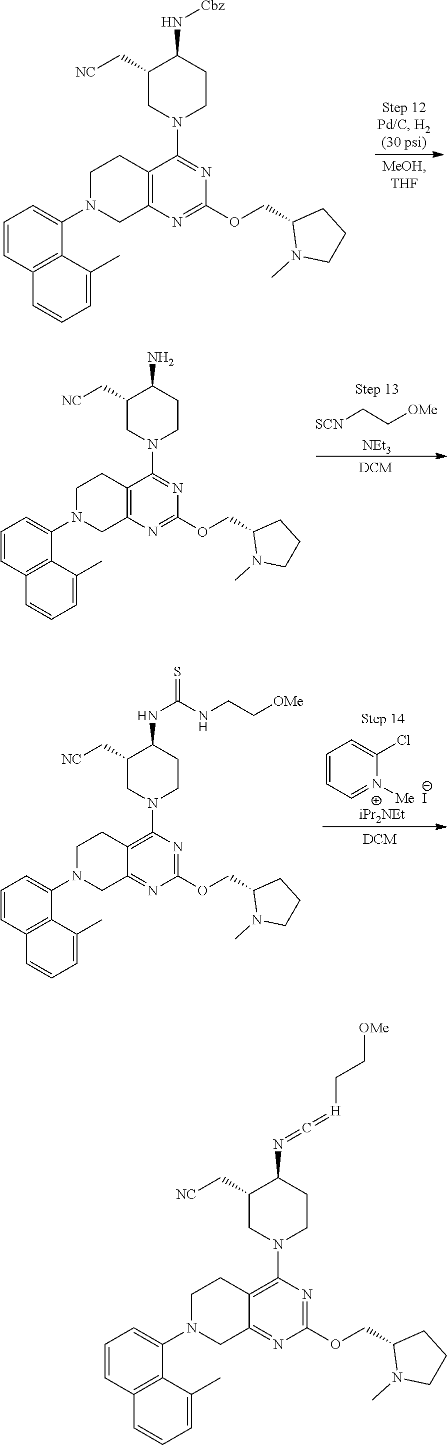

### Step 1: Synthesis of 1-(tert-butyl) 3-ethyl (S)-4-((1-phenylethyl)amino)-5,6-dihydropyridine-1,3(2H)-dicarboxylate

To a solution of 1-tert-butyl 3-ethyl 4-oxopiperidine-1,3-dicarboxylate (150 g, 553 mmol, 1 equiv) in toluene (1.5 L) was added (S)-1-phenylethanamine (75.0 g, 619 mmol, 1.12 equiv) and p-TsOH (4.32 g, 27.6 mmol, 0.05 equiv). The resulting mixture was heated to 135° C. with a Dean Stark trap for 12 h, then cooled to room temperature. The reaction was washed with sat. aq. NaHCO₃ (2×300 mL), and the organic phase was washed with sat. aq. NaCl (800 mL), dried over Na₂SO₄, filtered, and concentrated under reduced pressure. The crude residue was purified by silica gel column chromatography (0→10% EtOAc/petroleum ether) to afford 1-(tert-butyl) 3-ethyl (S)-4-((1-phenylethyl)amino)-5,6-dihydropyridine-1,3(2H)-dicarboxylate (190 g, 92% yield) as a pale yellow oil. ¹H NMR (400 MHz, CDCl₃) δ 9.24 (br d, J=7.5 Hz, 1H), 7.36-7.28 (m, 2H), 7.27-7.19 (m, 3H), 4.67-4.54 (m, 1H), 4.26-4.13 (m, 2H), 4.09-4.00 (m, 2H), 3.48-3.36 (m, 1H), 3.34-3.24 (m, 1H), 2.44-2.32 (m, 1H), 2.10-2.00 (m, 1H), 1.49 (d, J=6.8 Hz, 3H), 1.43 (s, 9H), 1.35-1.23 (m, 3H).

### Step 2: Synthesis of 1-(tert-butyl) 3-ethyl (3R,4S)-4-(((S)-1-phenylethyl)amino)piperidine-1,3-dicarboxylate

Two separate reactions were run in parallel. For each reaction, to a suspension of NaBH₄ (11.1 g, 294 mmol, 2 equiv) in THF (0.83 L) at 0° C. was added dropwise TFA (100 g, 881 mmol, 6 equiv). The resulting mixture was stirred for 10 min then cooled to −45° C. and a solution of 1-(tert-butyl) 3-ethyl (S)-4-((1-phenylethyl)amino)-5,6-dihydropyridine-1,3(2H)-dicarboxylate (55 g, 147 mmol, 1 equiv) in MeCN (275 mL) was added. The reaction was stirred for 1 h, then warmed to 0° C. and stirred for 1 h. The two separate reaction mixtures were combined, adjusted to pH 8 with 25% aq. NH₄OH, and concentrated under reduced pressure. The resulting yellow oil was dissolved in EtOAc (1 L) and H₂O (1 L), and 25% aq. NH₄OH (240 mL) was added. The resulting mixture was extracted into EtOAc (3×800 mL) and the combined organic phase was dried over Na₂SO₄, filtered and concentrated under reduced pressure. The resulting crude product was dissolved in MeCN (2.5 L) and filtered to afford 1-(tert-butyl) 3-ethyl (3R,4S)-4-(((S)-1-phenylethyl)amino)piperidine-1,3-dicarboxylate (30 g) as a white solid. The filtrate was concentrated under reduced pressure and purified by reverse phase chromatography (10→40% MeCN/H₂O, 0.1% TFA). Fractions containing the desired product were combined, adjusted to pH 7 with sat. aq. NaHCO₃, and concentrated under reduced pressure to remove MeCN. The resulting mixture was extracted into EtOAc (3×3.5 L), and the combined organic phase was washed with sat. aq. NaCl (2×1.5 L), dried over Na₂SO₄, filtered, and concentrated under reduced pressure to afford 1-(tert-butyl) 3-ethyl (3R,4S)-4-(((S)-1-phenylethyl)amino)piperidine-1,3-dicarboxylate (86 g, 78% yield) as a yellow oil. ¹H NMR (400 MHz, CDCl₆) δ 7.37-7.28 (m, 4H), 7.25 (dd, J=3.0, 5.6 Hz, 1H), 4.27-4.15 (m, 2H), 4.05-3.93 (m, 1H), 3.88 (q, J=6.4 Hz, 1H), 3.75-3.60 (m, 1H), 3.20 (dd, J=3.9, 13.8 Hz, 1H), 3.02 (ddd, J=3.7, 9.1, 13.2 Hz, 1H), 2.88 (td, J=4.1, 8.4 Hz, 1H), 2.78 (d, J=0.7 Hz, 1H), 1.83-1.71 (m, 1H), 1.57-1.49 (m, 1H), 1.47-1.42 (m, 9H), 1.37-1.23 (m, 6H).

### Step 3: Synthesis of (4S)-1-(tert-butoxycarbonyl)-4-(((S)-1-phenylethyl)amino)piperidine-3-carboxylic Acid

To a solution of Na (15.5 g, 674 mmol, 2.95 equiv) in EtOH (1.9 L) was added a solution of 1-(tert-butyl) 3-ethyl (3R,4S)-4-(((S)-1-phenylethyl)amino)piperidine-1,3-dicarboxylate (86 g, 228 mmol, 1 equiv) in EtOH (344 mL) and the resulting mixture was heated to 50° C. for 15 h then cooled to room temperature and concentrated under reduced pressure. The resulting residue was diluted with sat. aq. NaCl (1.5 L), extracted into EtOAc (3×1 L), and the combined organic phase was dried over Na₂SO₄, filtered and concentrated under reduced pressure to afford (4S)-1-(tert-butoxycarbonyl)-4-(((S)-1-phenylethyl)amino)piperidine-3-carboxylic acid as a yellow oil which was used without further purification.

### Step 4: Synthesis of 1-(tert-butyl) 3-ethyl (3S,4S)-4-(((S)-1-phenylethyl)amino)piperidine-1,3-dicarboxylate

To a solution of (4S)-1-(tert-butoxycarbonyl)-4-(((S)-1-phenylethyl)amino) piperidine-3-carboxylic acid (80 g, 230 mmol, 1 equiv) in DMF (800 mL) was added K₂CO₃ (34.9 g, 252 mmol, 1.1 equiv). The resulting mixture was stirred for 30 min then iodoethane (39.4 g, 252 mmol, 1.1 equiv) was added. After 12 h the reaction was poured into H₂O (4 L) and extracted into EtOAc (3×2 L). The combined organic phase was washed with sat. aq. NaCl (2×800 mL), dried over Na₂SO₄, filtered, and concentrated under reduced pressure. The resulting crude residue was purified by silica gel column chromatography (1→15% EtOAc/petroleum ether) followed by reverse phase reverse phase chromatography (10→40% MeCN/H₂O, 0.1% TFA). Fractions containing the desired product were combined, adjusted to pH 7 with sat. aq. NaHCO₃, and concentrated under reduced pressure to remove MeCN. The resulting aqueous phase was extracted into EtOAc (3×3.5 L) and the combined organic phase was washed with sat. aq. NaCl (2×1.5 L), dried over Na₂SO₄, filtered, and concentrated under reduced pressure. The resulting yellow oil was dissolved in MTBE (80 mL) and added dropwise to HCl (1 M in dioxane, 50.5 mL, 0.5 equiv) at 0° C. The resulting mixture was stirred for 30 min then heptane (96 mL) was added. After 1 h then mixture was filtered to afford a white solid. The filtrate was concentrated under reduced pressure to afford crude product (17 g) which was repurified. The isolated solid was washed with heptane, triturated with heptane (200 mL) and filtered, then dissolved in sat. aq. NaHCO₃, extracted into EtOAc (3×200 mL), and the combined organic phase was washed with sat. aq. NaCl (15 mL), dried over Na₂SO₄, filtered, and concentrated under reduced pressure to afford 1-(tert-butyl) 3-ethyl (3S,4S)-4-(((S)-1-phenylethyl)amino)piperidine-1,3-dicarboxylate (18 g) as a colorless oil. The crude product was repurified by the same method to afford 25 g total (27% yield). ¹H NMR (400 MHz, CDCl₃) δ 7.34-7.28 (m, 4H), 7.26-7.20 (m, 1H), 4.32-4.15 (m, 3H), 4.06-3.91 (m, 1H), 3.82 (q, J=6.5 Hz, 1H), 3.04-2.78 (m, 2H), 2.67 (t, J=12.3 Hz, 1H), 2.37-2.23 (m, 1H), 1.75 (dd, J=2.8, 6.3 Hz, 1H), 1.44 (s, 9H), 1.36-1.23 (m, 6H), 1.10 (d, J=12.1 Hz, 1H).

### Step 5: Synthesis of 1-(tert-butyl) 3-ethyl (3S,4S)-4-aminopiperidine-1,3-dicarboxylate

To a solution of 1-(tert-butyl) 3-ethyl (3S,4S)-4-(((S)-1-phenylethyl)amino)piperidine-1,3-dicarboxylate (18 g, 47.8 mmol, 1 equiv) in EtOH (72 mL) was added Pd/C (6 g, 10% purity). The resulting mixture was stirred under H₂ (50 psi) at 40° C. for 12 h, then filtered through celite, washed with EtOH (8×200 mL) and concentrated under reduced pressure to afford 1-(tert-butyl) 3-ethyl (3S,4S)-4-aminopiperidine-1,3-dicarboxylate (11.3 g, 87% yield) as pale yellow oil which was used without further purification.

### Step 6: Synthesis of 1-(tert-butyl) 3-ethyl (3S,4S)-4-(((benzyloxy)carbonyl)amino)piperidine-1,3-dicarboxylate

To a solution of 1-(tert-butyl) 3-ethyl (3S,4S)-4-aminopiperidine-1,3-dicarboxylate (11.3 g, 41.5 mmol, 1 equiv) in THF (110 mL) at 0° C. was added benzyl (2,5-dioxopyrrolidin-1-yl) carbonate (10.3 g, 41.5 mmol, 1 equiv). The resulting mixture was stirred for 15 min then poured into H₂O (200 mL) and extracted into EtOAc (3×80 mL). The combined organic phase was washed with sat. aq. NaCl (15 mL), dried over Na₂SO₄, filtered, and concentrated under reduced pressure. The residue was purified by silica gel column chromatography (10→25% EtOAc/petroleum ether) to afford 1-(tert-butyl) 3-ethyl (3S,4S)-4-(((benzyloxy)carbonyl)amino)piperidine-1,3-dicarboxylate (14.7 g, 78% yield) as a pale yellow oil. ¹H NMR (400 MHz, CDCl₃) δ 7.46-7.29 (m, 5H), 5.17-5.00 (m, 2H), 4.81 (s, 1H), 4.40-3.98 (m, 4H), 3.97-3.81 (m, 1H), 3.18-2.74 (m, 2H), 2.44-2.29 (m, 1H), 2.12-1.93 (m, 1H), 1.46 (s, 9H), 1.21 (t, J=7.2 Hz, 3H).

### Step 7: Synthesis of tert-butyl (3S,4S)-4-(((benzyloxy)carbonyl)amino)-3-(hydroxymethyl)piperidine-1-carboxylate

Two separate reactions were run in parallel. For each reaction, to a solution of 1-(tert-butyl) 3-ethyl (3S,4S)-4-(((benzyloxy)carbonyl)amino)piperidine-1,3-dicarboxylate (9 g, 22.1 mmol, 1 equiv) in THF (90 mL) at 0° C. was added slowly LiAlH₄ (1.26 g, 33.2 mmol, 1.5 equiv). The resulting mixture was stirred for 1 h then the two separate reactions were combined. H₂O (2.6 mL) was added dropwise followed by 15% aq. NaOH (2.6 mL) and the mixture was filtered. The filter cake was washed with EtOAc (6×50 mL) and the filtrate was diluted with H₂O (300 mL). The aqueous phase was extracted into EtOAc (3×100 mL) and the combined organic phase was washed with sat. aq. NaCl (20 mL), dried over Na₂SO₄, filtered, and concentrated under reduced pressure. The residue was purified by silica gel column chromatography (10→50% EtOAc/petroleum ether) to afford tert-butyl (3S,4S)-4-(((benzyloxy)carbonyl)amino)-3-(hydroxymethyl)piperidine-1-carboxylate (9.7 g, 53% yield) as colorless oil. ¹H NMR (400 MHz, CDCl₃) δ 7.46-7.30 (m, 5H), 5.19-5.08 (s, 2H), 4.71 (d, J=8.7 Hz, 1H), 4.19-3.96 (m, 2H), 3.82-3.63 (m, 2H), 3.54-3.42 (m, 1H), 3.31 (s, 1H), 3.00-2.65 (m, 2H), 1.98-1.85 (m, 1H), 1.46 (s, 9H).

### Step 8: Synthesis of tert-butyl (3S,4S)-4-(((benzyloxy)carbonyl)amino)-3-(((methylsulfonyl)oxy)methyl)piperidine-1-carboxylate

To a solution of tert-butyl (3S,4S)-4-(((benzyloxy)carbonyl)amino)-3-(hydroxymethyl)piperidine-1-carboxylate (12.7 g, 34.8 mmol, 1.0 equiv) in DCM (127 mL) at 0° C. was added NEt₃ (5.29 g, 52.3 mmol, 1.5 equiv) followed by MsCl (4.35 g, 38.0 mmol, 1.09 equiv). The resulting mixture was warmed to room temperature and stirred for 1 h then diluted with H₂O (200 mL) and extracted into DCM (3×100 mL). The combined organic phase was washed with sat. aq. NaCl (20 mL), dried over Na₂SO₄, filtered, and concentrated under reduced pressure to afford tert-butyl (3S,4S)-4-(((benzyloxy)carbonyl)amino)-3-(((methylsulfonyloxy)methylpiperidine-1-carboxylate which was used without further purification. ¹H NMR (400 MHz, CDCl₃) δ 7.46-7.30 (m, 5H), 5.22-4.99 (m, 2H), 4.69 (br d, J=8.8 Hz, 1H), 4.35-4.04 (m, 4H), 3.71-3.49 (m, 1H), 2.96 (s, 3H), 2.86-2.60 (m, 2H), 1.96 (dd, J=3.2, 13.0 Hz, 1H), 1.87-1.74 (m, 1H), 1.46 (s, 9H).

### Step 9: Synthesis of tert-butyl (3S,4S)-4-(((benzyloxy)carbonyl)amino)-3-(cyanomethyl)piperidine-1-carboxylate

To a solution of tert-butyl (3S,4S)-4-(((benzyloxy)carbonyl)amino)-3-(((methylsulfonyloxy)methylpiperidine-1-carboxylate (15.2 g, 34.3 mmol, 1.0 equiv) in DMA (228 mL) was added NaCN (3.37 g, 68.7 mmol, 2 equiv) and the resulting mixture was heated to 55° C. for 12 h then cooled to room temperature, poured into H₂O (1 L) at 0° C., and extracted into EtOAc (3×500 mL). The combined organic phase was washed with sat. aq. NaCl (3×200 mL), dried over Na₂SO₄, filtered, and concentrated under reduced pressure. The crude residue was purified by silica gel column chromatography (10→100% EtOAc/petroleum ether) to afford tert-butyl (3S,4S)-4-(((benzyloxy)carbonylamino)-3-(cyanomethyl)piperidine-1-carboxylate (10.2 g, 76% yield) as pale yellow oil. ¹H NMR (400 MHz, CDCl₃) δ 7.45-7.31 (m, 5H), 5.20-5.03 (m, 2H), 4.65 (d, J=8.5 Hz, 1H), 4.36-4.02 (m, 2H), 3.63-3.44 (m, 1H), 2.90-2.76 (m, 1H), 2.72-2.48 (m, 2H), 2.32 (dd, J=6.4, 15.4 Hz, 1H), 2.00-1.90 (m, 1H), 1.75 (s, 1H), 1.53-1.38 (m, 9H).

### Step 10: Synthesis of benzyl ((3S,4S)-3-(cyanomethyl)piperidin-4-yl)carbamate

To a solution of tert-butyl (3S,4S)-4-(((benzyloxy)carbonylamino)-3-(cyanomethyl)piperidine-1-carboxylate (3 g, 8.03 mmol, 1 equiv) in MeOH (15 mL) at 0° C. was added HCl (4M in MeOH, 60 mL, 30 equiv) and the resulting mixture was warmed to room temperature and stirred for 1 h then concentrated under reduced pressure to afford benzyl ((3S,4S)-3-(cyanomethyl)piperidin-4-yl)carbamate hydrochloride (2.49 g, 67% yield) as a white solid, which was used without further purification. LCMS (ESI) m/z: [M+H] calcd for C₁₅H₂₀N₃O₂: 274.15; found 274.2.

### Step 11: Synthesis of benzyl ((3S,4S)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)carbamate

To a solution of (S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate (4.1 g, 7.64 mmol, 1.0 equiv) and N,N-diisopropylethylamine (9.88 g, 76.4 mmol, 10 equiv) in DMF (41 mL) was added benzyl ((3S,4S)-3-(cyanomethyl)piperidin-4-yl)carbamate hydrochloride (2.49 g, 8.02 mmol, 1.05 equiv) and the resulting mixture was stirred for 40 min then slowly added to H₂O (500 mL) at 0° C. The resulting solid was filtered to afford benzyl ((3S,4S)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)carbamate as a yellow solid that was used without further purification. ¹H NMR (400 MHz, CDCl₃) δ 7.67 (dd, J=8.1, 19.7 Hz, 2H), 7.44-7.31 (m, 6H), 7.26-7.19 (m, 3H), 5.21-5.07 (m, 2H), 4.74-3.97 (m, 6H), 3.93-3.44 (m, 4H), 3.33-3.03 (m, 4H), 2.99-2.93 (m, 3H), 2.79-2.64 (m, 3H), 2.62-2.45 (m, 3H), 2.17-1.89 (m, 5H), 1.22-1.14 (m, 2H).

### Step 12: Synthesis of 2-((3S,4S)-4-amino-1-(7-(8-methyl)naphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile

To a solution of benzyl ((3S,4S)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)carbamate (7.8 g, 11.8 mmol, 1.0 equiv) in MeOH (25 mL) and THF (200 mL) was added Pd/C (3 g, 10% purity). The resulting mixture was stirred under H₂ (30 psi) for 0.5 h then Pd/C (1 g, 10% purity) was added to the mixture. After 0.5 h the reaction was filtered through celite, washed with MeOH (8×100 mL), and concentrated under reduced pressure. The crude residue was purified by reverse phase chromatography (35→65% MeCN/H₂O, 10 mM NH₄HCO₃) to afford 2-((3S,4S)-4-amino-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile (3.73 g, 60% yield) as white solid. ¹H NMR (400 MHz, Methanol-d₄) δ 7.67 (dd, J=8.1, 17.5 Hz, 20H), 7.41 (dt, J=3.9, 7.7 Hz, 1H), 7.35-7.21 (m, 3H), 4.50-4.14 (m, 4H), 4.07 (dd, J=7.3, 17.7 Hz, 1H), 3.74-3.60 (m, 1H), 3.56-3.46 (m, 1H), 3.29-3.11 (m, 3H), 3.10-3.03 (m, 1H), 3.01-2.94 (m, 1H), 2.92 (d, J=3.1 Hz, 3H), 2.89-2.52 (m, 5H), 2.50 (s, 3H), 2.34 (q, J=8.8 Hz, 1H), 2.15-1.91 (m, 2.5H), 1.86-1.62 (m, 4H), 1.47-1.34 (m, 0.5H).

### Step 13: Synthesis of 1-((3S,4S)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-3-(2-methoxyethyl)thiourea

To a solution of 2-((3S,4S)-4-amino-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile (50 mg, 95.1 μmol, 1 equiv) in DCM (951 μL) was added NEt₃ (79.3 μL, 570 μmol, 6 equiv) followed by 2-methoxyethyl isothiocyanate (11.2 μL, 104 μmol, 1.1 equiv). The resulting mixture was stirred for 21 h then diluted with DCM (20 mL), washed with H₂O (10 mL) followed by sat. aq. NaCl (10 mL), dried over Na₂SO₄, filtered, and concentrated under reduced pressure to afford 1-((3S,4S)-3-(cyanomethyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-3-(2-methoxyethyl)thiourea as a pale brown oil which was used without further purification. LCMS (ESI) m/z: [M+H] calcd for C₃₅H₄₇N₈O₂S: 643.35; found 643.4.

### Step 14: Synthesis of 2-((3S,4S)-4-((((2-methoxyethyl)imino)methylene)amino)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile

To a solution of 1-((3S,4S)-3-(cyanomethyl)-1-(7-(8-methylnaphthalen-1-yl)-2-(((S)-1-methylpyrrolidin-2-yl)(methoxy)-5,6,7,8-tetrahydropyrido[3,4-d]pyrimidin-4-yl)piperidin-4-yl)-3-(2-methoxyethyl)thiourea (61.0 mg, 95 μmol, 1 equiv) in DCM (949 μL) was added N,N-diisopropylethylamine (49.5 μL, 285 μmol, 3 equiv) followed by 2-chloro-1-methylpyridin-1-ium iodide (36.2 mg, 142 μmol, 1.5 equiv). The resulting mixture was stirred for 16 h then filtered to remove solids and concentrated under reduced pressure. The crude residue was purified by reverse phase chromatography (40→100% MeCN/H₂O, 0.4% NH₄OH) to afford 2-((3S,4S)-4-((((2-methoxyethyl)imino)methylene)amino)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile (27.1 mg, 47% yield over 2 steps) as a pale brown solid. LCMS (ESI) m/z: [M+H] calcd for C₃₅H₄₅N₈O₂: 609.37; found 609.4. ¹H NMR (500 MHz, Methanol-d₄) δ 7.70 (d, J=8.1 Hz, 1H), 7.66 (d, J=8.2 Hz, 1H), 7.42 (td, J=7.8, 4.2 Hz, 1H), 7.36-7.28 (m, 2H), 7.26 (d, J=7.0 Hz, 1H), 4.49-4.29 (m, 3H), 4.29-4.17 (m, 1H), 4.14-4.05 (m, 1H), 3.69 (dd, J=25.3, 17.8 Hz, 1H), 3.60-3.48 (m, 3H), 3.41-3.35 (m, 5H), 3.32-3.12 (m, 3H), 3.12-2.97 (m, 2H), 2.93 (d, J=5.6 Hz, 3H), 2.91-2.82 (m, 2H), 2.74 (dt, J=11.8, 6.8 Hz, 1H), 2.69-2.52 (m, 2H), 2.51 (s, 3H), 2.35 (q, J=9.0 Hz, 1H), 2.23-2.02 (m, 3H), 1.97-1.58 (m, 4H).

### Example 77—Synthesis of N-(2-methoxyethyl)-N-((1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methyl)methanediimine

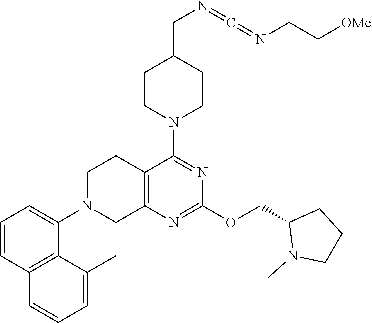 [SMILES: COCCN=C=NCC1CCN(c2nc(OC[C@@H]3CCCN3C)nc3c2CCN(c2cccc4cccc(C)c24)C3)CC1]

Synthesized according to the method of example 20, using tert-butyl (piperidin-4-ylmethyl)carbamate in place of tert-butyl piperidin-4-ylcarbamate in step 10. LCMS (ESI) m/z: [M+H] calcd for C₃₄H₄₆N₇O₂: 584.37; found 584.4.

### Example 78—Synthesis of N-((1r,4S)-4-((((2-methoxyethyl)imino)methylene)amino)cyclohexyl)-N-methyl-7-(8-methylnaphthalen-1-yl)-2-(((S)-1-methylpyrrolidin-2-yl)methoxy)-5,6,7,8-tetrahydropyrido[3,4-d]pyrimidin-4-amine

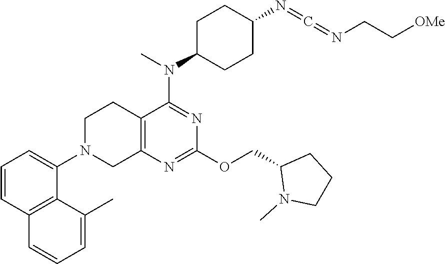 [SMILES: COCCN=C=N[C@H]1CC[C@H](N(C)c2nc(OC[C@@H]3CCCN3C)nc3c2CCN(c2cccc4cccc(C)c24)C3)CC1]

Synthesized according to the method of example 20, using tert-butyl ((1r,4r)-4-(methylamino)cyclohexyl)carbamate in place of tert-butyl piperidin-4-ylcarbamate in step 10. LCMS (ESI) m/z: [M+H] calcd for C₃₅H₄₈N₇O₂: 598.39; found 598.4.

### Example 79—Synthesis of N-((1s,4R)-4-((((2-methoxyethyl)imino)methylene)amino)cyclohexyl)-N-methyl-7-(8-methylnaphthalen-1-yl)-2-(((S)-1-methylpyrrolidin-2-yl)methoxy)-5,6,7,8-tetrahydropyrido[3,4-d]pyrimidin-4-amine

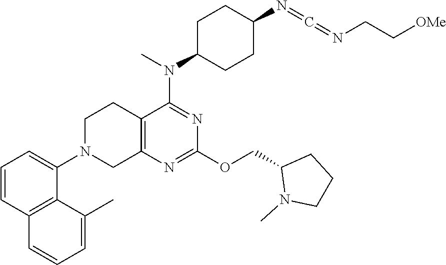 [SMILES: COCCN=C=N[C@H]1CC[C@@H](N(C)c2nc(OC[C@@H]3CCCN3C)nc3c2CCN(c2cccc4cccc(C)c24)C3)CC1]

Synthesized according to the method of example 20, using tert-butyl ((1s,4s)-4-(methylamino)cyclohexyl)carbamate in place of tert-butyl piperidin-4-ylcarbamate in step 10. LCMS (ESI) m/z: [M+H] calcd for C₃₅H₄₈N₇O₂: 598.39; found 598.4.

### Example 80—Synthesis of N-(1-(methoxymethyl)cyclopropyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

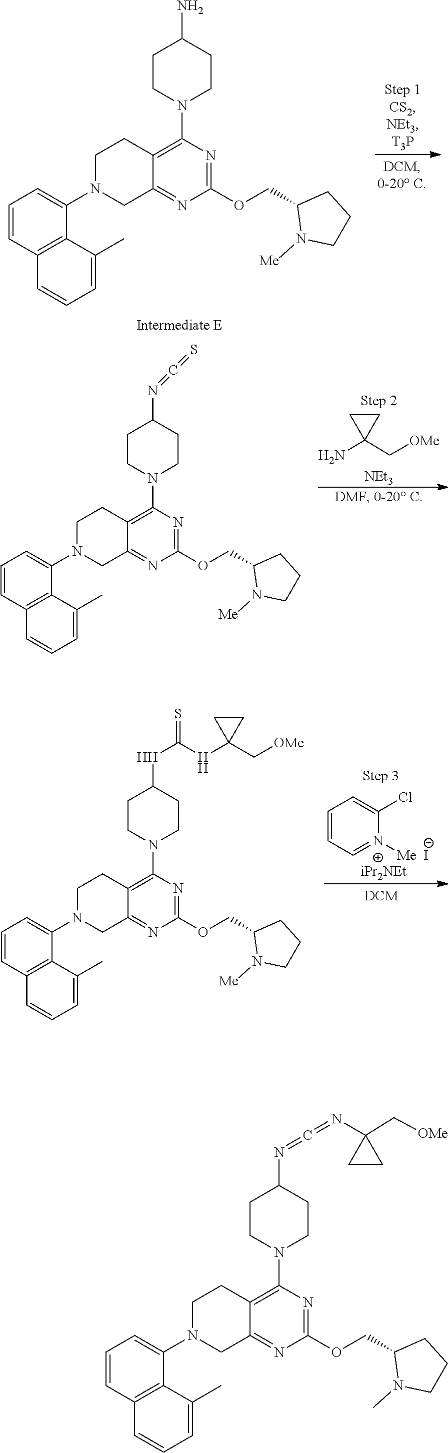

### Step 1: Synthesis of (S)-4-(4-isothiocyanatopiperidin-1-yl)-7-(8-methylnaphthalen-1-yl)-2-((1-methylpyrrolidin-2-yl)methoxy)-5,6,7,8-tetrahydropyrido[3,4-d]pyrimidine

To a solution of (S)-1-(7-(8-methylnaphthalen-1-yl)-2-((1-methylpyrrolidin-2-yl) methoxy)-5,6,7,8-tetrahydropyrido[3,4-d]pyrimidin-4-yl)piperidin-4-amine hydrochloride (4.2 g, 8.03 mmol, 1 equiv) and NEt₃ (4.06 g, 40.1 mmol, 5 equiv) in DCM (60 mL) at 0° C. was added a mixture of NEt₃ (4.06 g, 40.1 mmol, 5 equiv) and CS₂ (1.83 g, 24.1 mmol, 3 equiv). The resulting mixture was stirred at room temperature for 0.5 h, then cooled to 0° C. and T₃P (50%, 9.20 g, 14.4 mmol, 1.8 equiv) was added. The reaction was stirred at room temperature for 0.5 h then extracted into DCM (3×30 mL). The combined organic phase was washed with sat. aq. NH₄Cl (3×30 mL) then sat. aq. NaCl (40 mL), dried over Na₂SO₄, filtered, and concentrated under reduced pressure. The resulting crude yellow solid was used without further purification.

### Step 2: Synthesis of (S)-1-(1-(methoxymethyl)cyclopropyl)-3-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)thiourea

To a solution of (S)-4-(4-isothiocyanatopiperidin-1-yl)-7-(8-methylnaphthalen-1-yl)-2-((1-methylpyrrolidin-2-yl)methoxy)-5,6,7,8-tetrahydropyrido[3,4-d]pyrimidine (600 mg, 1.13 mmol, 1 equiv) in DMF (6 mL) was added NEt₃ (287 mg, 2.84 mmol, 2.5 equiv). The resulting mixture was cooled to 0° C. and 1-(methoxymethyl)cyclopropan-1-amine hydrochloride (172 mg, 1.25 mmol, 1.1 equiv) was added. The reaction was stirred for 3 h then quenched with H₂O (60 mL) and extracted into DCM (3×20 mL). The combined organic phase was washed with sat. aq. NH₄Cl (3×30 mL) then sat. aq. NaCl (30 mL), dried over Na₂SO₄, filtered, and concentrated under reduced pressure. The crude product was purified by reverse phase chromatography (55-75% MeCN/H₂O, 0.05% NH₄OH+10 mM NH₄HCO₃) to afford (S)-1-(1-(methoxymethyl)cyclopropyl)-3-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)thiourea (190 mg, 26% yield) as a white solid. LCMS (ESI) m/z: [M+H] calcd for C₃₅H₄₈N₇O₂S: 630.35; found 630.4. ¹H NMR (400 MHz, Methanol-d₄) δ 7.69 (d, J=7.8 Hz, 1H), 7.66-7.60 (m, 1H), 7.41 (t, J=7.7 Hz, 1H), 7.35-7.27 (m, 2H), 7.27-7.21 (m, 1H), 4.48 (s, 1H), 4.42-4.33 (m, 1H), 4.33-4.23 (m, 1H), 4.20-3.96 (m, 3H), 3.66 (d, J=17.7 Hz, 1H), 3.56-3.40 (m, 3H), 3.39-3.33 (m, 4H), 3.23-3.11 (m, 3H), 3.07 (td, J=4.6, 9.7 Hz, 1H), 2.92 (s, 3H), 2.81-2.69 (m, 1H), 2.68-2.57 (m, 1H), 2.49 (d, J=0.9 Hz, 3H), 2.34 (q, J=9.0 Hz, 1H), 2.23-2.01 (m, 3H), 1.89-1.65 (m, 4H), 1.64-1.49 (m, 1H), 1.01-0.81 (m, 4H).

### Step 3: Synthesis of N-(1-(methoxymethyl)cyclopropyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

To a solution of (S)-1-(1-(methoxymethyl)cyclopropyl)-3-(1-(7-(8-methylnaphthalen-1-yl)-2-((1-methylpyrrolidin-2-yl)methoxy)-5,6,7,8-tetrahydropyrido[3,4-d]pyrimidin-4-yl)piperidin-4-yl)thiourea (70 mg, 111 μmol, 1 equiv) in DCM (1.1 mL) was added N,N-diisopropylethylamine (57.9 μL, 333 μmol, 3 equiv) followed by 2-chloro-1-methylpyridin-1-ium iodide (42.4 mg, 166 μmol, 1.5 equiv). The resulting mixture was stirred for 16 h then filtered to remove solids and concentrated under reduced pressure. The crude residue was purified by reverse phase chromatography (40→100% MeCN/H₂O, 0.4% NH₄OH) to afford N-(1-(methoxymethyl)cyclopropyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine (27.5 mg, 42%) as a yellow oil. LCMS (ESI) m/z: [M+H] calcd for C₃₅H₄₆N₇O₂: 596.37; found 596.4. ¹H NMR (500 MHz, Methanol-d₄) δ 7.70 (d, J=8.1 Hz, 1H), 7.65 (d, J=8.2 Hz, 1H), 7.42 (t, J=7.7 Hz, 1H), 7.36-7.28 (m, 2H), 7.25 (d, J=7.0 Hz, 1H), 4.38 (ddd, J=13.1, 10.9, 6.0 Hz, 1H), 4.29 (ddd, J=16.9, 10.9, 5.8 Hz, 1H), 4.12-4.03 (m, 2H), 3.97 (d, J=13.8 Hz, 1H), 3.70-3.58 (m, 2H), 3.54-3.49 (m, 1H), 3.47 (s, 2H), 3.41-3.35 (m, 4H), 3.25-3.13 (m, 3H), 3.08 (dt, J=9.6, 4.6 Hz, 1H), 2.93 (s, 3H), 2.74 (p, J=6.9 Hz, 1H), 2.66-2.56 (m, 1H), 2.50 (s, 3H), 2.35 (q, J=9.0 Hz, 1H), 2.15-2.06 (m, 2H), 2.05-1.98 (m, 1H), 1.87-1.76 (m, 3H), 1.76-1.58 (m, 2H), 0.78 (s, 4H).

### Example 81—Synthesis of N-(2-methoxybenzyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

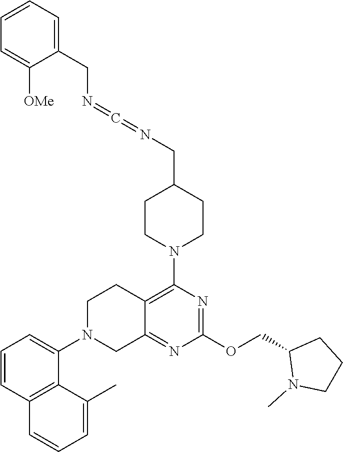 [SMILES: COc1ccccc1CN=C=NCC1CCN(c2nc(OC[C@@H]3CCCN3C)nc3c2CCN(c2cccc4cccc(C)c24)C3)CC1]

Synthesized according to the method of example 20, using 2-methoxybenzyl isothiocyanate in place of 2-methoxyethyl isothiocyanate in step 12. LCMS (ESI) m/z: [M+H] calcd for C₃₈H₄₆N₇O₂: 632.37; found 632.4.

### Example 82—Synthesis of N-(2,4-dimethoxybenzyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

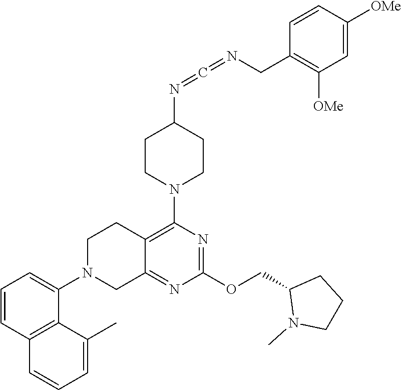 [SMILES: COc1ccc(CN=C=NC2CCN(c3nc(OC[C@@H]4CCCN4C)nc4c3CCN(c3cccc5cccc(C)c35)C4)CC2)c(OC)c1]

Synthesized according to the method of example 80, using (2,4-dimethoxyphenyl)methanamine in place of 1-(methoxymethyl)cyclopropan-1-amine in step 2. LCMS (ESI) m/z: [M+H] calcd for C₃₉H₄₈N₇O₃: 662.38; found 662.4.

### Example 83—Synthesis of N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-N-(1-methylpiperidin-4-yl)methanediimine

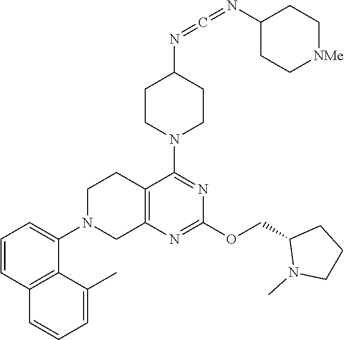 [SMILES: Cc1cccc2cccc(N3CCc4c(nc(OC[C@@H]5CCCN5C)nc4N4CCC(N=C=NC5CCN(C)CC5)CC4)C3)c12]

Synthesized according to the method of example 80, using 1-methylpiperidin-4-amine in place of 1-(methoxymethyl)cyclopropan-1-amine in step 2. LCMS (ESI) m/z: [M+H] calcd for C₃₆H₄₉N₈O: 609.40; found 609.4.

### Example 84—Synthesis of N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-N-(1-phenylpiperidin-4-yl)methanediimine

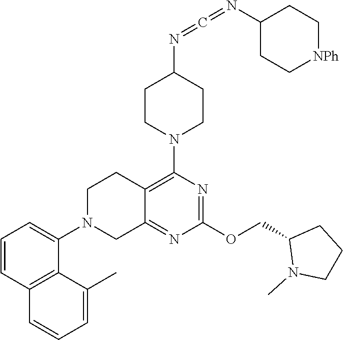

Synthesized according to the method of example 80, using 1-phenylpiperidin-4-amine in place of 1-(methoxymethyl)cyclopropan-1-amine in step 2. LCMS (ESI) m/z: [M+H] calcd for C₄₁H₅₁N₈O: 671.42; found 671.4.

### Example 85—Synthesis of N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-N-(3-morpholinopropyl)methanediimine

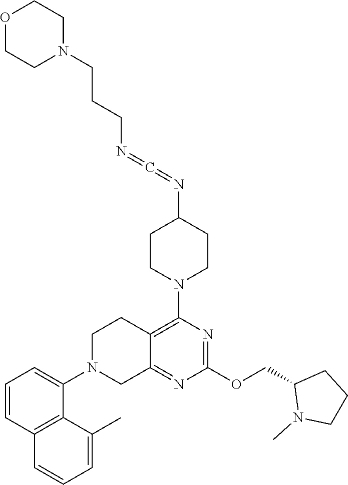 [SMILES: Cc1cccc2cccc(N3CCc4c(nc(OC[C@@H]5CCCN5C)nc4N4CCC(N=C=NCCCN5CCOCC5)CC4)C3)c12]

Synthesized according to the method of example 20, using 4-(3-isothiocyanatopropyl)morpholine in place of 2-methoxyethyl isothiocyanate in step 12. LCMS (ESI) m/z: [M+H] calcd for C₃₇H₅₁N₈O₂: 639.41; found 639.5.

### Example 86—Synthesis of N-(isoxazol-3-ylmethyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

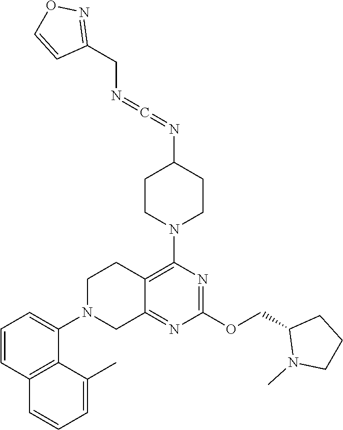 [SMILES: Cc1cccc2cccc(N3CCc4c(nc(OC[C@@H]5CCCN5C)nc4N4CCC(N=C=NCc5ccon5)CC4)C3)c12]

Synthesized according to the method of example 20, using 3-(isothiocyanatomethyl)isoxazole in place of 2-methoxyethyl isothiocyanate in step 12. LCMS (ESI) m/z: [M+H] calcd for C₃₄H₄₁N₈O₂: 593.34; found 593.4.

### Example 87—Synthesis of N-(2-(1H-imidazol-1-yl)ethyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

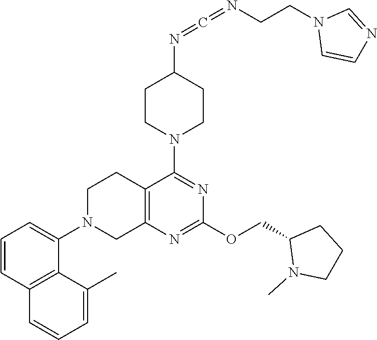 [SMILES: Cc1cccc2cccc(N3CCc4c(nc(OC[C@@H]5CCCN5C)nc4N4CCC(N=C=NCCn5ccnc5)CC4)C3)c12]

Synthesized according to the method of example 80, using 2-(1H-imidazol-1-yl)ethan-1-amine in place of 1-(methoxymethyl)cyclopropan-1-amine in step 2. LCMS (ESI) m/z: [M+H] calcd for C₃₅H₄₄N₉O: 606.37; found 606.4.

### Example 88—Synthesis of N-(3-(1H-imidazol-1-yl)propyl)-N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)methanediimine

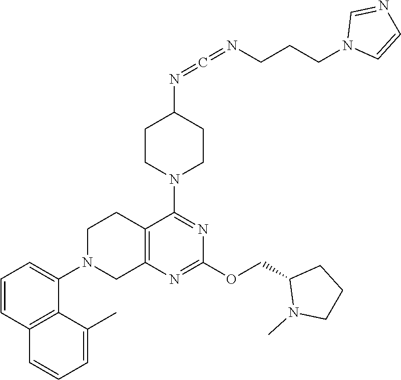 [SMILES: Cc1cccc2cccc(N3CCc4c(nc(OC[C@@H]5CCCN5C)nc4N4CCC(N=C=NCCCn5ccnc5)CC4)C3)c12]

Synthesized according to the method of exaample 80, using 3-(1H-imidazol-1-yl)propan-1-amine in place of 1-(methoxymethyl)cyclopropan-1-amine in step 2. LCMS (ESI) m/z: [M+H] calcd for C₃₆H₄₆N₉O: 620.38; found 620.4.

### Example 89—Synthesis of N-methyl-N-(2-((((1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)imino)methylene)amino)ethyl)aniline

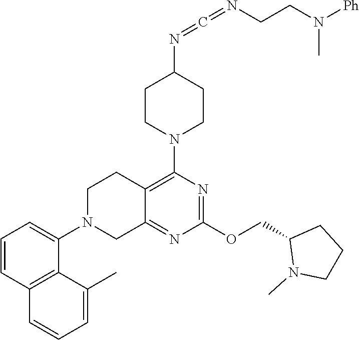 [SMILES: Cc1cccc2cccc(N3CCc4c(nc(OC[C@@H]5CCCN5C)nc4N4CCC(N=C=NCCN(C)c5ccccc5)CC4)C3)c12]

Synthesized according to the method of example 80, using N¹-methyl-N¹-phenylethane-1,2-diamine in place of 1-(methoxymethyocyclopropan-1-amine in step 2. LCMS (ESI) m/z: [M+H] calcd for C₃₉H₄₉N₈O: 645.40; found 645.4.

### Example 90—Synthesis of N-methyl-N-(3-((((1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)imino)methylene)amino)propyl)aniline

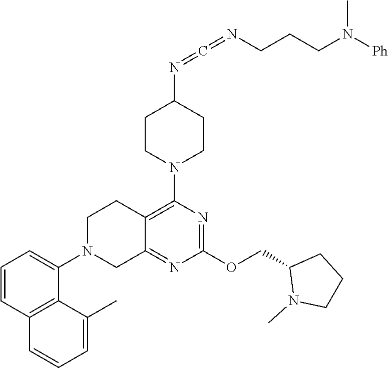 [SMILES: Cc1cccc2cccc(N3CCc4c(nc(OC[C@@H]5CCCN5C)nc4N4CCC(N=C=NCCCN(C)c5ccccc5)CC4)C3)c12]

Synthesized according to the method of example 80, using M-methyl-M-phenylpropane-1,3-diamine in place of 1-(methoxymethyl)cyclopropan-1-amine in step 2. LCMS (ESI) m/z: [M+H] calcd for C₄₀H₅₁N₈O: 659.42; found 659.4.

### Example 91—Synthesis of N-methyl-N-(3-((((1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)imino)methylene)amino)propyl)pyridin-2-amine

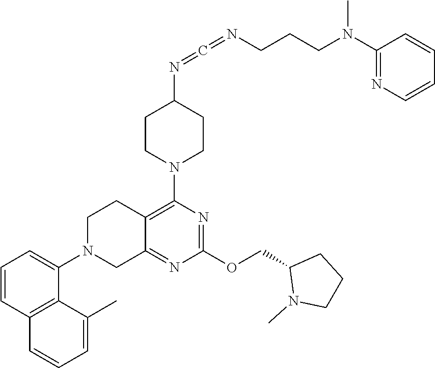 [SMILES: Cc1cccc2cccc(N3CCc4c(nc(OC[C@@H]5CCCN5C)nc4N4CCC(N=C=NCCCN(C)c5ccccn5)CC4)C3)c12]

Synthesized according to the method of example 80, using N¹-methyl-N¹-(pyridin-2-yl)propane-1,3-diamine in place of 1-(methoxymethyl)cyclopropan-1-amine in step 2. LCMS (ESI) m/z: [M+H] calcd for C₃₉H₅₀N₉O: 660.41; found 660.4.

### Example 92—Synthesis of N-methyl-N-(2-((((1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)imino)methylene)amino)ethyl)pyrimidin-4-amine

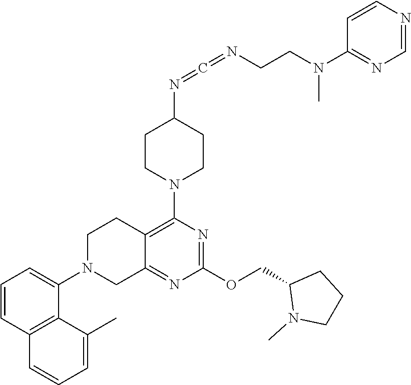 [SMILES: Cc1cccc2cccc(N3CCc4c(nc(OC[C@@H]5CCCN5C)nc4N4CCC(N=C=NCCN(C)c5ccncn5)CC4)C3)c12]

Synthesized according to the method of example 80, using N¹-methyl-N¹-(pyrimidin-4-yl)ethane-1,2-diamine in place of 1-(methoxymethyl)cyclopropan-1-amine in step 2. LCMS (ESI) m/z: [M+H] calcd for C₃₇H₄₇N₁₀O: 647.39; found 647.4.

### Example 93—Synthesis of N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-N-(3-(methylthio)propyl)methanediimine

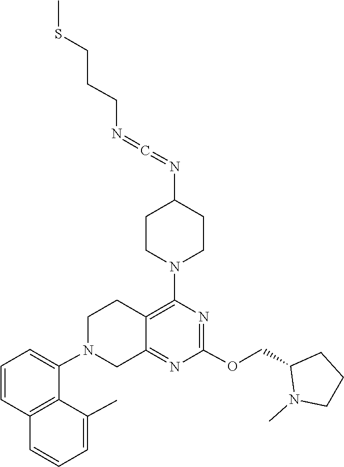 [SMILES: CSCCCN=C=NC1CCN(c2nc(OC[C@@H]3CCCN3C)nc3c2CCN(c2cccc4cccc(C)c24)C3)CC1]

Synthesized according to the method of example 20, using (3-isothiocyanatopropyl)(methyl)sulfane in place of 2-methoxyethyl isothiocyanate in step 12. LCMS (ESI) m/z: [M+H] calcd for C₃₄H₄₈N₇OS: 600.35; found 600.4.

### Example 94—Synthesis of N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-N-(thiophen-2-ylmethyl)methanediimine

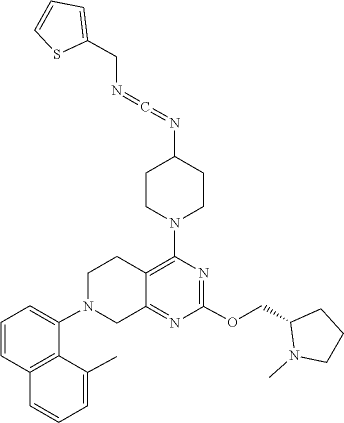 [SMILES: Cc1cccc2cccc(N3CCc4c(nc(OC[C@@H]5CCCN5C)nc4N4CCC(N=C=NCc5cccs5)CC4)C3)c12]

Synthesized according to the method of example 20, using 2-(isothiocyanatomethyl)thiophene in place of 2-methoxyethyl isothiocyanate in step 12. LCMS (ESI) m/z: [M+H] calcd for C₃₅H₄₂N₇OS: 608.32; found 608.3.

### Example 95—Synthesis of N-(1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-4-yl)-N-(2-(thiophen-2-yl)ethyl)methanediimine

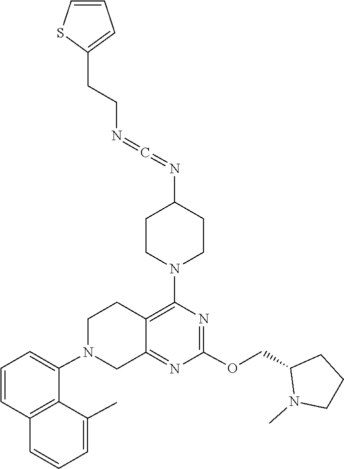 [SMILES: Cc1cccc2cccc(N3CCc4c(nc(OC[C@@H]5CCCN5C)nc4N4CCC(N=C=NCCc5cccs5)CC4)C3)c12]

Synthesized according to the method of example 20, using 2-(2-isothiocyanatoethyl)thiophene in place of 2-methoxyethyl isothiocyanate in step 12. LCMS (ESI) m/z: [M+H] calcd for C₃₆H₄₄N₇OS: 622.33; found 622.3.

### Example 96—Synthesis of 2-((S)-1-((R)-1-benzylaziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

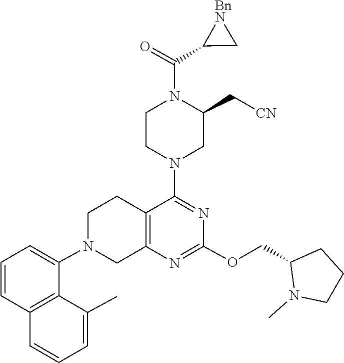 [SMILES: Cc1cccc2cccc(N3CCc4c(nc(OC[C@@H]5CCCN5C)nc4N4CCN(C(=O)[C@H]5CN5Cc5ccccc5)[C@@H](CC#N)C4)C3)c12]

Synthesized according to the method of reaction scheme 4, using example 63 in place of compound 1 and benzyl bromide in place of compound 2. LCMS (ESI) m/z: [M+H] calcd for C₄₀H₄₇N₈O₂: 671.38; found 671.4.

### Example 97—Synthesis of 2-((S)-1-((S)-1-benzylaziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

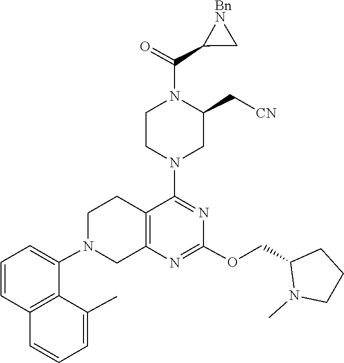 [SMILES: Cc1cccc2cccc(N3CCc4c(nc(OC[C@@H]5CCCN5C)nc4N4CCN(C(=O)[C@@H]5CN5Cc5ccccc5)[C@@H](CC#N)C4)C3)c12]

Synthesized according to the method of reaction scheme 4, using example 64 in place of compound 1 and benzyl bromide in place of compound 2. LCMS (ESI) m/z: [M+H] calcd for C₄₀H₄₇N₈O₂: 671.38; found 671.4.

### Example 98—Synthesis of 2-((S)-1-((R)-1-acetylaziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

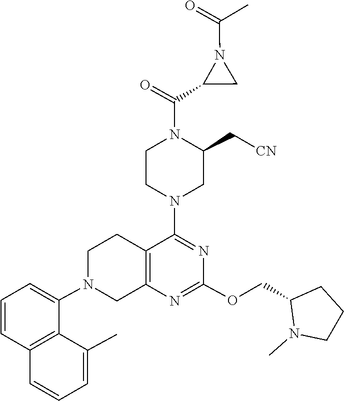 [SMILES: CC(=O)N1C[C@@H]1C(=O)N1CCN(c2nc(OC[C@@H]3CCCN3C)nc3c2CCN(c2cccc4cccc(C)c24)C3)C[C@@H]1CC#N]

Synthesized according to the method of example 43, using example 63 in place of example 42. LCMS (ESI) m/z: [M+H] calcd for C₃₅H₄₃N₈O₃: 623.35; found 623.4.

### Example 99—Synthesis of 2-((S)-1-((S)-1-acetylaziridine-2-carbonyl)-4-(7-(8-methylnaphthaien-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

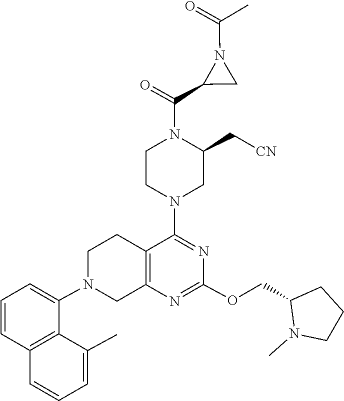 [SMILES: CC(=O)N1C[C@H]1C(=O)N1CCN(c2nc(OC[C@@H]3CCCN3C)nc3c2CCN(c2cccc4cccc(C)c24)C3)C[C@@H]1CC#N]

Synthesized according to the method of example 43, using example 64 in place of example 42. LCMS (ESI) m/z: [M+2H]/2 calcd for C₃₅H₄₄N₈O₃: 312.17; found 312.3.

### Example 100—Synthesis of 2-((S)-1-(((R)-aziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

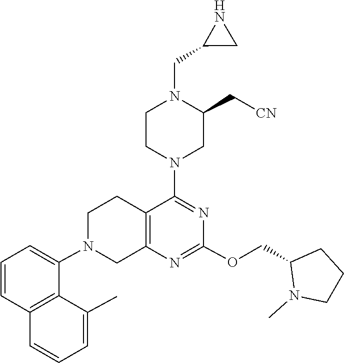 [SMILES: Cc1cccc2cccc(N3CCc4c(nc(OC[C@@H]5CCCN5C)nc4N4CCN(C[C@H]5CN5)[C@@H](CC#N)C4)C3)c12]

Synthesized according to the method of reaction scheme 2, using Intermediate F in place of compound 1 in step 1. LCMS (ESI) m/z: [M+H] calcd for C₃₃H₄₃N₈O: 567.36; found 567.4.

### Example 101—Synthesis of 2-((S)-1-(((S)-aziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

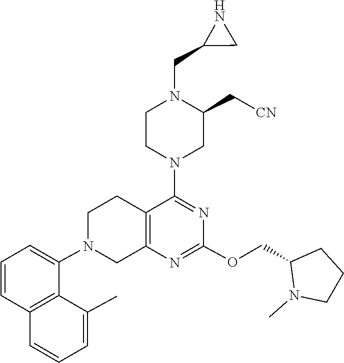 [SMILES: Cc1cccc2cccc(N3CCc4c(nc(OC[C@@H]5CCCN5C)nc4N4CCN(C[C@@H]5CN5)[C@@H](CC#N)C4)C3)c12]

Synthesized according to the method of reaction scheme 2, using Intermediate F in place of compound 1 in step 1. LCMS (ESI) m/z: [M+H] calcd for C₃₃H₄₃N₈O: 567.36; found 567.4.

### Example 102—Synthesis of 2-((3S,4R)-4-((((2-methoxybenzyl)imino)methylene)amino)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile

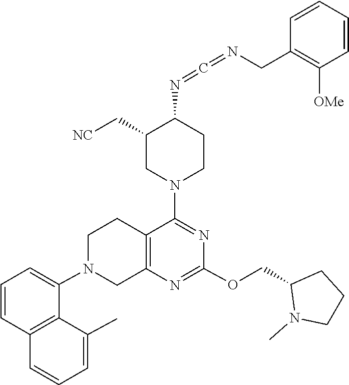 [SMILES: COc1ccccc1CN=C=N[C@@H]1CCN(c2nc(OC[C@@H]3CCCN3C)nc3c2CCN(c2cccc4cccc(C)c24)C3)C[C@@H]1CC#N]

Synthesized according to the method of example 75, using 2-methoxybenzyl isothiocyanate in place of 2-methoxyethyl isothiocyanate in step 11. LCMS (ESI) m/z: [M+H] calcd for C₄₀H₄₇N₈O₂: 671.38; found 671.4.

### Example 103—Synthesis of 2-((3S,4S)-4-((((2-methoxybenzyl)imino)methylene)amino)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperidin-3-yl)acetonitrile

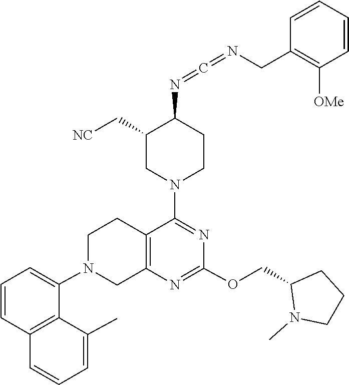 [SMILES: COc1ccccc1CN=C=N[C@H]1CCN(c2nc(OC[C@@H]3CCCN3C)nc3c2CCN(c2cccc4cccc(C)c24)C3)C[C@@H]1CC#N]

Synthesized according to the method of example 76, using 2-methoxybenzyl isothiocyanate in place of 2-methoxyethyl isothiocyanate in step 13. LCMS (ESI) m/z: [M+H] calcd for C₄₀H₄₇N₈O₂: 671.38; found 671.5.

### Example 104—Synthesis of N-((1R,3S)-3-((((2-methoxybenzyl)imino)methylene)amino)cyclobutyl)-7-(8-methylnaphthalen-1-yl)-2-(((S)-1-methylpyrrolidin-2-yl)methoxy)-5,6,7,8-tetrahydropyrido[3,4-d]pyrimidin-4-amine

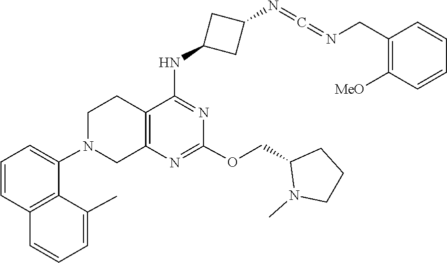 [SMILES: COc1ccccc1CN=C=N[C@H]1C[C@H](Nc2nc(OC[C@@H]3CCCN3C)nc3c2CCN(c2cccc4cccc(C)c24)C3)C1]

Synthesized according to the method of example 20, using tert-butyl ((1R,3R)-3-aminocyclobutyl)carbamate in place of tert-butyl piperidin-4-ylcarbamate in step 10, and 2-methoxybenzyl isothiocyanate in place of 2-methoxyethyl isothiocyanate in step 12. LCMS (ESI) m/z: [M+H] calcd for C₃₇H₄₄N₇O₂: 618.36; found 618.4.

### Example 105—Synthesis of 2-((S)-1-((R)-1-(2-methoxyethyl)aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

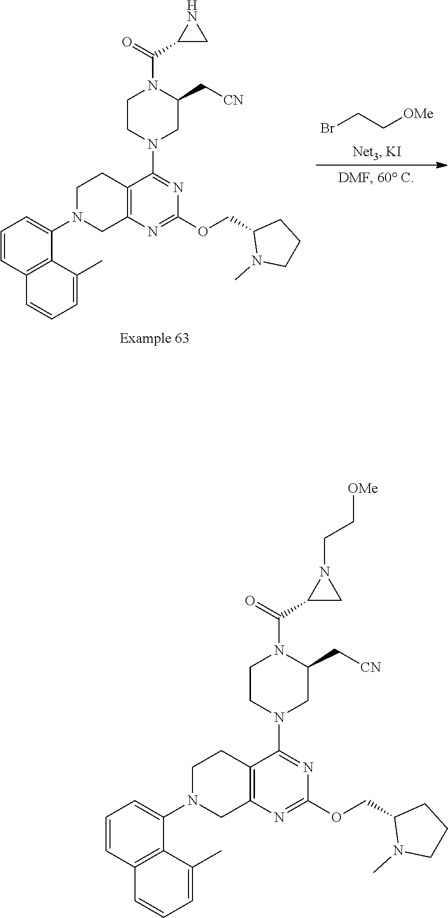 [SMILES: Cc1cccc2cccc(N3CCc4c(nc(OC[C@@H]5CCCN5C)nc4N4CCN(C(=O)[C@H]5CN5)[C@@H](CC#N)C4)C3)c12 ; COCCBr ; COCCN1C[C@@H]1C(=O)N1CCN(c2nc(OC[C@@H]3CCCN3C)nc3c2CCN(c2cccc4cccc(C)c24)C3)C[C@@H]1CC#N]

To a solution of 2-((S)-1-((R)-aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (400 mg, 689 μmol, 1 equiv) and 1-bromo-2-methoxyethane (97 μL, 1.03 mmol, 1.5 equiv) in DMF (4 mL) was added NEt₃ (960 μL, 6.89 mmol, 10 equiv) and KI (57.2 mg, 344 μmol, 0.5 equiv). The resulting mixture was heated to 60° C. After 12 h the reaction was cooled to room temperature, filtered, then purified by reverse phase chromatography (50→75% MeOH/H₂O, 10 mM NH₄HCO₃) to afford 2-((S)-1-((R)-1-(2-methoxyethyl)aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (45.1 mg, 10% yield) as a white solid. LCMS (ESI) m/z: [M+H] calcd for C₃₆H₄₇N₈O₃: 639.38; found 639.4. ¹H NMR (400 MHz, CDCl₃) δ 7.76-7.57 (m, 2H), 7.47 (d, J=0.9 Hz, 1H), 7.34 (t, J=7.7 Hz, 1H), 7.27-7.17 (m, 2H), 5.11-4.82 (m, 1H), 4.64-4.01 (m, 6H), 3.99-3.61 (m, 3H), 3.58-3.34 (m, 5H), 3.32-3.05 (m, 4H), 3.02-2.87 (m, 4H), 2.86-2.57 (m, 4H), 2.57-2.42 (m, 3H), 2.31 (s, 2H), 2.22-1.99 (m, 1H), 1.96-1.51 (m, 5H).

### Example 106—Synthesis of 2-((S)-1-((S)-1-(2-methoxyethyl)aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

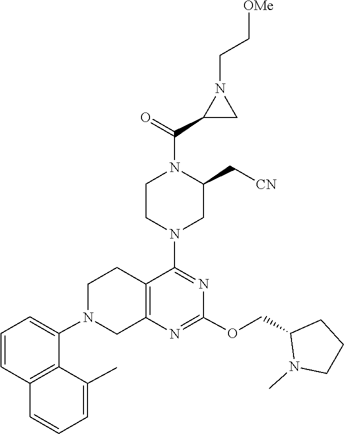 [SMILES: COCCN1C[C@H]1C(=O)N1CCN(c2nc(OC[C@@H]3CCCN3C)nc3c2CCN(c2cccc4cccc(C)c24)C3)C[C@@H]1CC#N]

Synthesized according to the method of example 105, using example 64 in place of example 63. LCMS (ESI) m/z: [M+H] calcd for C₃₆H₄₇N₈O₃: 639.38; found 639.4.

### Example 107—Synthesis of 2-((S)-1-((S)-1-methylaziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

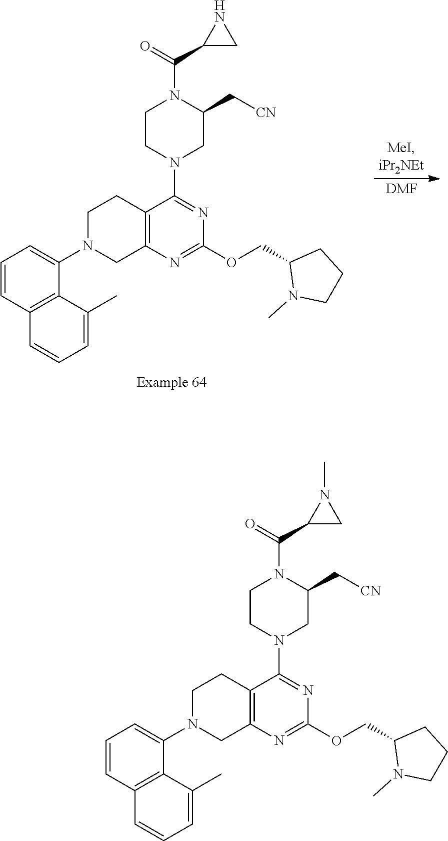

A solution of2-((S)-1-((S)-aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (500 mg, 861 μmol, 1 equiv), MeI (54 μL, 861 μmol, 1 equiv), and N,N-diisopropylethylamine (450 μL, 2.58 mmol, 3 equiv) in DMF (5 mL) was stirred at room temperature for 2 h then filtered. The resulting filtrate was purified by reverse phase chromatography (20→40% MeCN/H₂O, 0.2% formic acid) to afford a white solid then repurified by reverse phase chromatography (25→45% MeCN/H₂O, 10 mM NH₄HCO₃) to afford 2-((S)-1-((S)-1-methylaziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (40.7 mg, 8% yield) as a white solid. LCMS (ESI) m/z: [M+H] calcd for C₃₄H₄₃N₈O₂: 595.35; found 595.4. ¹H NMR (400 MHz, CDCl₃) δ 8.85 (s, 1H), 7.73-7.59 (m, 2H), 7.47-7.37 (m, 1H), 7.33 (t, J=7.5 Hz, 1H), 7.29 (s, 1H), 7.26-7.11 (m, 1H), 5.21-5.03 (m, 1H), 5.22-4.76 (m, 1H), 4.43-4.03 (m, 4H), 4.75-4.03 (m, 2H), 4.00-3.83 (m, 2H), 4.00-3.64 (m, 2H), 3.47 (d, J=10.6 Hz, 5H), 3.34-3.08 (m, 7H), 3.06-2.96 (m, 1H), 2.96-2.86 (m, 3H), 2.85-2.42 (m, 3H), 2.39-2.01 (m, 3H), 2.00-1.76 (m, 2H).

### Example 108—Synthesis of methyl (R)-1-((S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl)aziridine-2-carboxylate

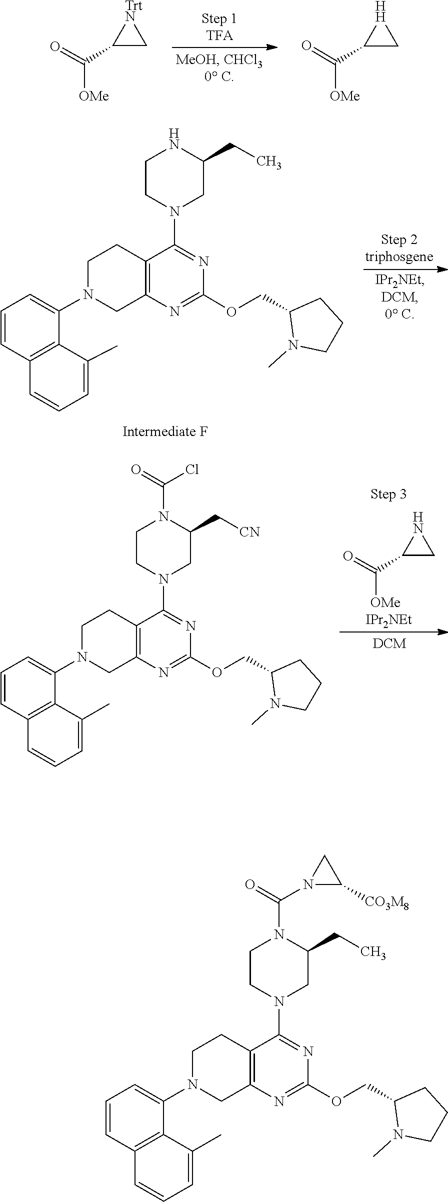

### Step 1: Synthesis of methyl (R)-aziridine-2-carboxylate

To a solution of methyl (R)-1-tritylaziridine-2-carboxylate (500 mg, 1.48 mmol, 1 equiv) in MeOH (2.5 mL) and CHCl₃ (2.5 mL) at 0° C. was added TFA (1.67 mL, 21.84 mmol, 15 equiv). The resulting mixture was stirred for 1 h then quenched with H₂O (5 mL) and extracted into DCM (4×2 mL). The combined organic phase was washed with sat. aq. NaCl (3 mL), dried over Na₂SO₄, filtered, and concentrated under reduced pressure to afford methyl (R)-aziridine-2-carboxylate (200 mg, crude) as white solid which was used without further purification.

### Step 2: Synthesis of (S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl chloride

To a solution of 2-((S)-4-(7-(8-methylnaphthalen-1-yl)-2-(((S)-1-methylpyrrolidin-2-yl)-methoxy)-5,6,7,8-tetrahydropyrido[3,4-d]pyrimidin-4-yl)piperazin-2-yl)acetonitrile (200 mg, 391 μmol, 1 equiv) in DCM (2 mL) at 0° C. was added N,N-diisopropylethylamine (341 μL, 1.95 mmol, 5 equiv) and triphosgene (69.6 mg, 234 μmol, 0.6 equiv). The resulting mixture was stirred for 1 h then used directly in the next step.

### Step 3: Synthesis of methyl (R)-1-((S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl)aziridine-2-carboxylate

To a solution of methyl (R)-aziridine-2-carboxylate (108 mg, 1.05 mmol, 3 equiv) in DCM (1 mL) at 0° C. was added N,N-diisopropylethylamine (606 μL, 3.48 mmol, 10 equiv) followed by a solution of (S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl chloride in DCM (1 mL). The resulting mixture was stirred for 30 min then quenched with sat. aq. NH₄Cl (2 mL) and extracted into DCM (4×2 mL). The combined organic phase was washed with sat. aq. NaCl (3 mL), dried over Na₂SO₄, filtered, and concentrated under reduced pressure. The resulting crude product was purified by reverse phase chromatography (40→70% MeCN/H₂O, 10 mM NH₄HCO₃) then repurified by reverse phase chromatography (45→70% MeCN/H₂O, 0.05% NH₄OH+10 mM NH₄HCO₃) to afford methyl (R)-1-((S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl)aziridine-2-carboxylate (59.1 mg, 26% yield) as white solid. LCMS (ESI) m/z: [M+H] calcd for C₃₅H₄₃N₈O₄: 639.34; found 639.4. ¹H NMR (400 MHz, CDCl₃) δ 7.71-7.69 (m, 2H), 7.65-7.33 (m, 2H), 7.27-7.20 (m, 2H), 4.83 (s, 1H), 4.38-4.29 (m, 3H), 4.24-4.18 (m, 3H), 3.87-3.82 (m, 1H), 3.81 (s, 3H), 3.19-3.16 (m, 1H), 3.11-3.10 (m, 1H), 3.08-3.07 (m, 5H), 3.06-3.05 (m, 1H), 2.92 (s, 3H), 2.66-2.65 (m, 5H), 2.48 (s, 3H), 1.78-1.76 (m, 1H), 1.75-1.74 (m, 1H), 1.74-1.73 (m, 3H), 1.64 (s, 1H).

### Example 109—Synthesis of methyl (S)-1-((S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl)aziridine-2-carboxylate

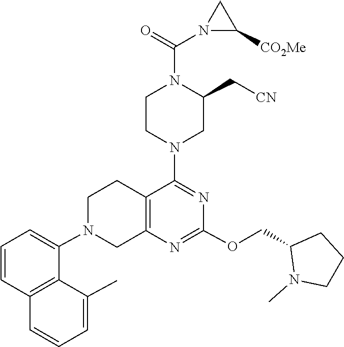 [SMILES: COC(=O)[C@@H]1CN1C(=O)N1CCN(c2nc(OC[C@@H]3CCCN3C)nc3c2CCN(c2cccc4cccc(C)c24)C3)C[C@@H]1CC#N]

Synthesized according to the method of example 108, using methyl (S)-1-tritylaziridine-2-carboxylate in place of methyl (R)-1-tritylaziridine-2-carboxylate in step 1. LCMS (ESI) m/z: [M+H] calcd for C₃₅H₄₃N₈O₄: 639.34; found 639.4.

### Example 110—Synthesis of 2-((S)-1-((R)-aziridine-2-carbonyl)-4-(7-(5-methyl-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile

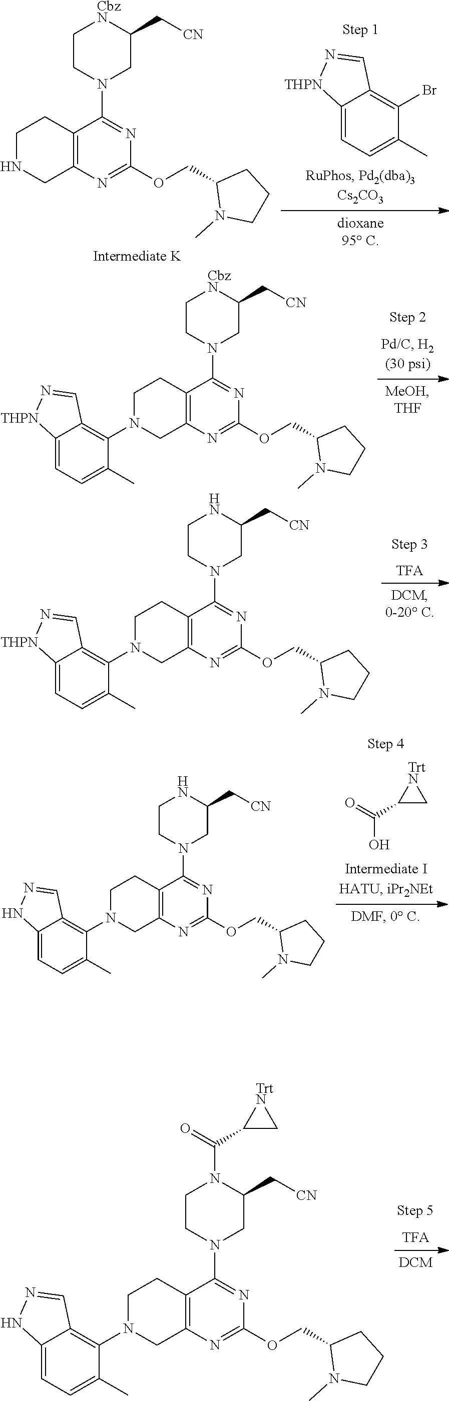

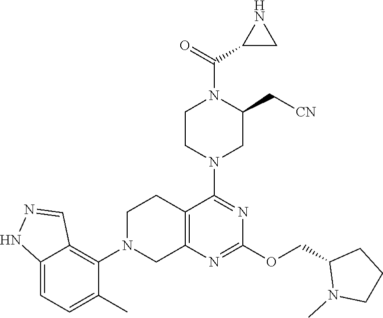 [SMILES: Cc1ccc2[nH]ncc2c1N1CCc2c(nc(OC[C@@H]3CCCN3C)nc2N2CCN(C(=O)[C@H]3CN3)[C@@H](CC#N)C2)C1]

### Step 1: Synthesis of benzyl (2S)-2-(cyanomethyl)-4-(7-(5-methyl-1-(tetrahydro-2H-pyran-2-yl)-1H-indazol-4-yl)-2-(((S)-1-methylpyrrolidin-2-yl)methoxy)-5,6,7,8-tetrahydropyrido[3,4-d]pyrimidin-4-yl)piperazine-1-carboxylate

To a solution of (S)-benzyl 2-(cyanomethyl)-4-(2-(((S)-1-methylpyrrolidin-2-yl)methoxy)-5,6,7,8-tetrahydropyrido[3,4-d]pyrimidin-4-yl)piperazine-1-carboxylate (7 g, 13.8 mmol, 1 equiv) in dioxane (105 mL) was added 4-bromo-5-methyl-1-(tetrahydro-2H-pyran-2-yl)-1H-indazole (8.17 g, 27.7 mmol, 2 equiv), RuPhos (1.29 g, 2.77 mmol, 0.2 equiv), Pd₂(dba)₃ (1.90 g, 2.08 mmol, 0.15 equiv) and Cs₂CO₃ (11.3 g, 34.6 mmol, 2.5 equiv). The resulting mixture was heated to 95° C. After 4 h the reaction was cooled to room temperature, filtered through Celite, and washed with DCM (4×30 mL). The filtrate was washed with H₂O (2×70 mL), sat. aq. NaCl (70 mL), dried over Na₂SO₄, filtered, and concentrated under reduced pressure. The resulting residue was purified by silica gel column chromatography (50→100% EtOAc/petroleum ether) to afford benzyl (2S)-2-(cyanomethyl)-4-(7-(5-methyl-1-(tetrahydro-2H-pyran-2-yl)-1H-indazol-4-yl)-2-(((S)-1-methylpyrrolidin-2-yl)methoxy)-5,6,7,8-tetrahydropyrido[3,4-d]pyrimidin-4-yl)piperazine-1-carboxylate (7.35 g, 63% yield) as an orange solid. ¹H NMR (400 MHz, Methanol-d₄) δ 8.07 (s, 1H), 7.41-7.34 (m, 5H), 7.29 (br t, J=8.3 Hz, 2H), 5.74 (br d, J=8.3 Hz, 1H), 5.26-5.12 (m, 2H), 4.69 (br s, 1H), 4.33 (dq, J=5.9, 11.2 Hz, 2H), 4.21 (s, 2H), 4.16-4.02 (m, 3H), 3.99 (br d, J=11.6 Hz, 1H), 3.84-3.74 (m, 1H), 3.53-3.45 (m, 2H), 3.28 (br s, 1H), 3.13-3.03 (m, 2H), 3.00-2.69 (m, 5H), 2.50 (s, 4H), 2.43-2.28 (m, 4H), 2.16-2.03 (m, 2H), 1.98 (br d, J=13.2 Hz, 1H), 1.87-1.76 (m, 3H), 1.75-1.56 (m, 4H).

### Step 2: Synthesis of 2-((2S)-4-(7-(5-methyl-1-(tetrahydro-2H-pyran-2-yl)-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of benzyl (2S)-2-(cyanomethyl)-4-(7-(5-methyl-1-(tetrahydro-2H-pyran-2-yl)-1H-indazol-4-yl)-2-(((S)-1-methylpyrrolidin-2-yl)methoxy)-5,6,7,8-tetrahydropyrido[3,4-d]pyrimidin-4-yl)piperazine-1-carboxylate (7 g, 9.72 mmol, 1 equiv) in MeOH (35 mL) and THF (35 mL) was added Pd/C (4.2 g, 10% purity) and the resulting mixture was stirred under H₂ (30 psi). After 2.5 h the reaction mixture was filtered through celite and concentrated under reduced pressure to afford 2-((2S)-4-(7-(5-methyl-1-(tetrahydro-2H-pyran-2-yl)-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile (5.6 g, 80% yield) as brown solid. ¹H NMR (400 MHz, Methanol-d₄) δ 8.08 (s, 1H), 7.41-7.34 (m, 1H), 7.32-7.26 (m, 1H), 5.75 (dd, J=2.3, 9.8 Hz, 1H), 4.41-4.34 (m, 1H), 4.34-4.27 (m, 1H), 4.24-4.12 (m, 3H), 4.03-3.92 (m, 2H), 3.79 (dt, J=3.1, 11.0 Hz, 1H), 3.52-3.45 (m, 2H), 3.22-3.10 (m, 2H), 3.10-3.00 (m, 2H), 2.96-2.84 (m, 2H), 2.84-2.69 (m, 3H), 2.65 (d, J=6.5 Hz, 2H), 2.55-2.45 (m, 4H), 2.40 (s, 3H), 2.37-2.25 (m, 1H), 2.16-2.02 (m, 2H), 1.98 (br dd, J=2.7, 13.2 Hz, 1H), 1.88-1.76 (m, 3H), 1.76-1.57 (m, 4H).

### Step 3: Synthesis of 2-((S)-4-(7-(5-methyl-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of 2-((2S)-4-(7-(5-methyl-1-(tetrahydro-2H-pyran-2-yl)-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile (4 g, 6.83 mmol, 1 equiv) in DCM (40 mL) at 0° C. was added TFA (10.5 mL, 137 mmol, 20 equiv) and the resulting mixture was warmed to room temperature. After 2 h, the reaction was poured into a mixture of ice and sat. aq. NaHCO₃ then extracted into DCM (3×50 mL). The combined organic phase was washed with sat. aq. NaCl (50 mL), dried over Na₂SO₄, filtered, and concentrated under reduced pressure. The resulting residue was purified by reverse phase chromatography (0→30% MeCN/H₂O, 0.1% TFA) to afford 2-((S)-4-(7-(5-methyl-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile (3.07 g, TFA salt) as a brown solid. LCMS (ESI) m/z: [M+H] calcd for C₂₇H₃₆N₉O: 502.30; found: 502.3. ¹H NMR (400 MHz, Methanol-d₄) δ 8.08 (s, 1H), 7.28 (s, 2H), 4.76 (dd, J=3.2, 12.6 Hz, 1H), 4.59 (dd, J=7.4, 12.7 Hz, 1H), 4.48 (br d, J=14.2 Hz, 1H), 4.29 (s, 2H), 4.25 (br d, J=15.0 Hz, 1H), 4.03-3.92 (m, 1H), 3.88 (br s, 1H), 3.74 (br s, 1H), 3.62-3.49 (m, 4H), 3.46-3.33 (m, 3H), 3.24 (br d, J=8.3 Hz, 1H), 3.09 (br s, 1H), 3.08 (s, 3H), 2.97 (br d, J=14.8 Hz, 1H), 2.89-2.79 (m, 1H), 2.42 (s, 3H), 2.41-2.33 (m, 1H), 2.27-2.17 (m, 1H), 2.16-1.98 (m, 2H).

### Step 4: Synthesis of 2-((S)-4-(7-(5-methyl-1H-indazol-4-yl)-2-(((S)-1-methylpyrrolidin-2-yl)methoxy)-5,6,7,8-tetrahydropyrido[3,4-d]pyrimidin-4-yl)-1-((R)-1-tritylaziridine-2-carbonyl)piperazin-2-yl)acetonitrile

To a suspension of (R)-1-tritylaziridine-2-carboxylic acid lithium salt (39.9 mg, 119 μmol, 1.2 equiv), 2-((S)-4-(7-(5-methyl-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile (50 mg, 99.6 μmol, 1 equiv), and HATU (45.2 mg, 119 μmol, 1.2 equiv) in DMF (1 mL) at 0° C. was added N,N-diisopropylethylamine (52 μL, 298 μmol, 3 equiv). The resulting mixture was stirred for 3 h then diluted with EtOAc (10 mL). The organic phase was washed with 5% aq. citric acid (20 mL), sat. aq. NaHCO₃ (20 mL), sat. aq. NaCl (10 mL), dried over Na₂SO₄, filtered, and concentrated under reduced pressure. The crude product was taken on without further purification.

### Step 5: Synthesis of 2-((S)-1-((R)-aziridine-2-carbonyl)-4-(7-(5-methyl-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of 2-((S)-4-(7-(5-methyl-1H-indazol-4-yl)-2-(((S)-1-methylpyrrolidin-2-yl)methoxy)-5,6,7,8-tetrahydropyrido[3,4-d]pyrimidin-4-yl)-1-((R)-1-tritylaziridine-2-carbonyl)piperazin-2-yl)acetonitrile (80 mg, 98.3 μmol, 1 equiv) in DCM (0.5 mL) at 0° C. was added TFA (150 μL, 2.0 mmol, 20 equiv). The resulting mixture was stirred for 10 min then quenched with MeOH (0.5 mL) followed by NEt₃ (273 μL, 1.96 mmol, 20 equiv) and concentrated under reduced pressure. The residue was purified by reverse phase chromatography (10→80% MeCN/H₂O, 0.4% NH₄OH) to afford 2-((S)-1-((R)-aziridine-2-carbonyl)-4-(7-(5-methyl-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile (10.0 mg, 18% yield over 2 steps) as a white solid. LCMS (ESI) m/z: [M+H] calcd for C₃₀H₃₉N₁₀O₂: 571.33; found 571.8. ¹H NMR (500 MHz, Methanol-d₄) δ 8.11 (d, J=3.1 Hz, 1H), 7.32-7.24 (m, 2H), 5.08-4.90 (m, 2H), 4.45-4.09 (m, 7H), 3.67 (t, J=12.8 Hz, 1H), 3.57 (t, J=5.3 Hz, 2H), 3.44-3.35 (m, 2H), 3.31-3.23 (m, 1H), 3.14-2.84 (m, 6H), 2.77 (q, J=7.1 Hz, 1H), 2.52 (s, 3H), 2.44 (s, 3H), 2.37 (q, J=9.0 Hz, 1H), 2.12 (dq, J=12.8, 8.3 Hz, 1H), 1.96-1.79 (m, 4H), 1.73 (dq, J=14.2, 7.2 Hz, 1H).

### Example 111—Synthesis of 2-((S)-1-((S)-aziridine-2-carbonyl)-4-(7-(5-methyl-1H-indazol-4-yl)-2-(((S)-1-methylpyrrolidin-2-yl)methoxy)-5,6,7,8-tetrahydropyrido[3,4-d]pyrimidin-4-yl)piperazin-2-yl)acetonitrile

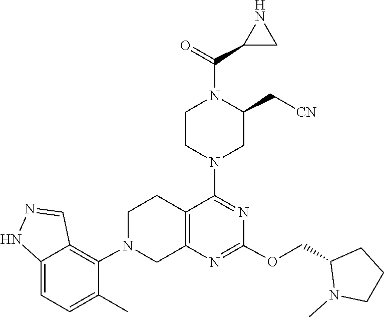 [SMILES: Cc1ccc2[nH]ncc2c1N1CCc2c(nc(OC[C@@H]3CCCN3C)nc2N2CCN(C(=O)[C@@H]3CN3)[C@@H](CC#N)C2)C1]

Synthesized according to the method of example 110, using (S)-1-tritylaziridine-2-carboxylic acid lithium salt in place of (R)-1-tritylaziridine-2-carboxylic acid lithium salt in step 4. LCMS (ESI) m/z: [M+H] calcd for C₃₀H₃₉N₁₀O₂: 571.33; found 571.8.

### Example 112—Synthesis of 2-((S)-1-((R)-aziridine-2-carbonyl)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

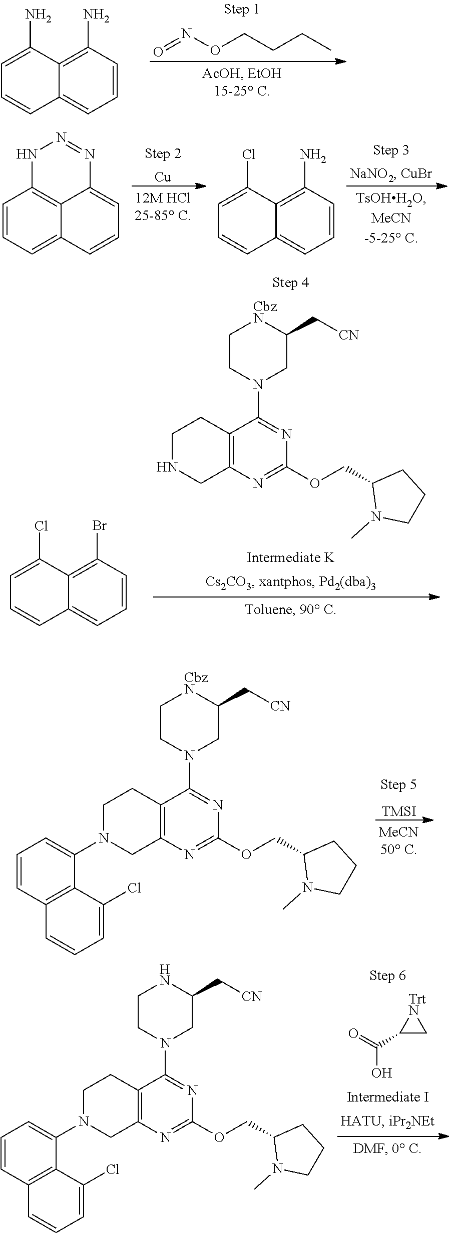

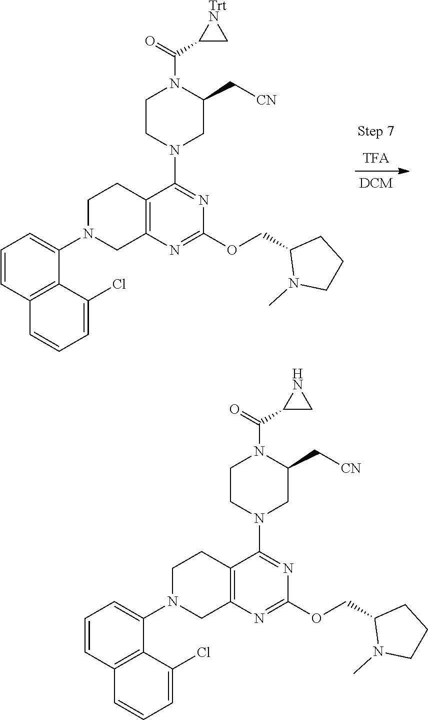

### Step 1: Synthesis of 1H-naphtho[1,8-de][1,2,3]triazine

To a solution of naphthalene-1,8-diamine (15 g, 95 mmol, 1 equiv) in AcOH (30 mL) and EtOH (150 mL) at 15° C. was added butyl nitrite (12.5 mL, 93 mmol, 0.98 equiv) dropwise, keeping the temperature between 15-20° C. The mixture was warmed to room temperature. After 3 h the reaction mixture was filtered and the filter cake was washed with EtOH (3×25 mL), petroleum ether (25 mL), and dried under reduced pressure to afford 1H-naphtho[1,8-de][1,2,3]triazine (11.5 g, 72% yield) as a red solid. ¹H NMR (400 MHz, DMSO-d₆) δ 13.26 (br s, 1H), 7.27 (br s, 2H), 6.96-7.18 (m, 2H), 6.89 (br s, 1H), 6.14 (br d, J=7.21 Hz, 1H).

### Step 2: Synthesis of 8-chloronaphthalen-1-amine

To a solution of 1H-naphtho[1,8-de][1,2,3]triazine (22.7 g, 134 mmol, 1 equiv) in HCl (12 N, 480 mL) was added Cu (588 mg, 8.91 mmol, 0.066 equiv). After 12 h the reaction mixture was diluted with H₂O (50 mL) and heated to 85° C. After 30 min the solution was filtered, cooled, and basified to pH 8-9 with sat. aq. NH₄OH. The reaction mixture was extracted into EtOAc (3×50 mL) then the combined organic phase was washed with sat. aq. NaCl (50 mL), dried over Na₂SO₄, filtered, and concentrated under reduced pressure. The crude residue was purified by silica gel column chromatography (2→17% EtOAc/petroleum ether) to afford 8-chloronaphthalen-1-amine (18.5 g, 78% yield) as a red solid. ¹H NMR (400 MHz, CDCl₃) δ 7.56 (d, J=8.07 Hz, 1H), 7.25-7.31 (m, 1H), 7.11-7.20 (m, 3H), 6.63 (dd, J=7.09, 1.47 Hz, 1H).

### Step 3: Synthesis of 1-bromo-8-chloronaphthalene

To a solution of 8-chloronaphthalen-1-amine (20.2 g, 114 mmol, 1 equiv) and TsOH.H₂O (77.9 g, 409 mmol, 3.6 equiv) in MeCN (360 mL) at −5° C. was added NaNO₂ (14.12 g, 205 mmol, 1.8 equiv) followed by a solution of CuBr (10.4 mL, 341 mmol, 3 equiv) in H₂O (48 mL). The reaction mixture was warmed to room temperature. After 12 h sat. aq. Na₂SO₃ (200 mL) was added. After 30 min of stirring the reaction mixture was concentrated under reduced pressure to remove organic solvents. The aqueous phase was extracted into EtOAc (3×80 mL), then the combined organic phase was washed with sat. aq. NaCl (80 mL), dried over Na₂SO₄, filtered, and concentrated under reduced pressure. The crude residue was purified by silica gel column chromatography (3→5% EtOAc/petroleum ether) to afford 1-bromo-8-chloronaphthalene (17.2 g, 63% yield) as yellow solid. ¹H NMR (400 MHz, CDCl₃) δ 7.93 (dd, J=7.46, 1.22 Hz, 1H), 7.80 (ddd, J=12.35, 8.19, 0.98 Hz, 2H), 7.67 (dd, J=7.52, 1.28 Hz, 1H), 7.38 (t, J=7.83 Hz, 1H), 7.25-7.32 (m, 1H).

### Step 4: Synthesis of benzyl (S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-2-(cyanomethyl)piperazine-1-carboxylate

To a solution of benzyl (S)-2-(cyanomethyl)-4-(2-(((S)-1-methylpyrrolidin-2-yl)methoxy)-5,6,7,8-tetrahydropyrido[3,4-d]pyrimidin-4-yl)piperazine-1-carboxylate (3.0 g, 5.9 mmol, 1 equiv) in toluene (80 mL) under N₂ was added 1-bromo-8-chloronaphthalene (4.3 g, 17.7 mmol, 3 equiv), Cs₂CO₃ (5.8 g, 17.7 mmol, 3 equiv), Pd₂(dba)₃ (815 mg, 890 μmol, 0.15 equiv) and xantphos (687 mg, 1.2 mmol, 0.2 equiv). The heterogeneous mixture was heated to 90° C. After 12 h the suspension was filtered and washed with EtOAc (3×50 mL). The combined filtrate was washed with H₂O (50 mL), sat. aq. NaCl (50 mL), dried over Na₂SO₄, filtered, and concentrated under reduced pressure. The crude residue was purified by silica gel column chromatography (0→10% MeOH/EtOAc) to afford benzyl (S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-2-(cyanomethyl)piperazine-1-carboxylate (2.6 g, 66% yield) as yellow solid. ¹H NMR (400 MHz, CDCl₃) δ 7.81 (d, J=8.16 Hz, 1H), 7.67 (dd, J=8.27, 3.20 Hz, 1H), 7.50 (dd, J=16.76, 7.50 Hz, 2H), 7.25-7.45 (m, 9H), 5.09-5.27 (m, 2H), 4.71 (br s, 1H), 4.25-4.39 (m, 3H), 4.07-4.15 (m, 2H), 3.62-3.75 (m, 1H), 3.38-3.61 (m, 2H), 2.97-3.29 (m, 6H), 2.58-2.96 (m, 3H), 2.51 (d, J=4.41 Hz, 3H), 2.32-2.42 (m, 1H), 2.05-2.15 (m, 1H), 1.77-1.88 (m, 2H), 1.62-1.74 (m, 1H).

### Step 5: Synthesis of 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of benzyl (S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-2-(cyanomethyl)piperazine-1-carboxylate (3.50 g, 5.25 mmol, 1 equiv) in MeCN (35 mL) was added TMSI (2.50 mL, 18.4 mmol, 3.5 equiv) and heated to 50° C. After 2 h the reaction mixture was cooled to room temperature and quenched with MeOH (20 mL). After 15 min of stirring the reaction mixture was poured into 0° C. HCl (1 N, 100 mL) and extracted into EtOAc (3×20 mL). The aqueous layer was basified to pH 8-9 with 0° C. NaOH (1 N) then extracted into EtOAc (3×30 mL). The combined organic phase was washed with sat. aq. NaCl (50 mL), dried over Na₂SO₄, filtered, and concentrated under reduced pressure. The crude residue was purified by reverse phase chromatography (15→45% MeCN/H₂O, 0.2% formic acid) to afford 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (1.81 g, 65% yield) as white solid. LCMS (ESI) m/z: [M+H] calcd for C₂₉H₃₅ClN₇O: 532.26; found 532.3; ¹H NMR (400 MHz, Methanol-d₄) δ 7.77-7.82 (m, 1H), 7.65 (d, J=8.19 Hz, 1H), 7.41-7.53 (m, 2H), 7.31-7.37 (m, 1H), 7.28 (dd, J=6.72, 3.67 Hz, 1H), 4.20-4.41 (m, 4H), 3.95-4.08 (m, 1H), 3.89 (br d, J=13.20 Hz, 0.5H), 3.66 (br dd, J=17.36, 13.57 Hz, 1H), 3.52 (br dd, J=5.99, 3.67 Hz, 1H), 2.91-3.29 (m, 7.5H), 2.72-2.87 (m, 2H), 2.61-2.70 (m, 2H), 2.48-2.60 (m, 4H), 2.37 (qd, J=8.91, 3.85 Hz, 1H), 1.99-2.14 (m, 1H), 1.76-1.85 (m, 2H), 1.66-1.76 (m, 1H).

### Step 6: Synthesis of 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((R)-1-tritylaziridine-2-carbonyl)piperazin-2-yl)acetonitrile

To a suspension of (R)-1-tritylaziridine-2-carboxylic acid lithium salt (34.5 mg, 103 μmol, 1.1 equiv), 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (50 mg, 93.9 μmol, 1 equiv), and HATU (39.1 mg, 103 μmol, 1.1 equiv) in DMF (0.9 mL) was added N,N-diisopropylethylamine (49 μL, 281 μmol, 3 equiv). The resulting mixture was stirred for 2 h then diluted with EtOAc (10 mL). The organic phase was washed with 5% aq. citric acid (20 mL), sat. aq. NaHCO₃ (20 mL), sat. aq. NaCl (10 mL), dried over Na₂SO₄, filtered, and concentrated under reduced pressure. The crude product was taken on without further purification.

### Step 7: Synthesis of 2-((S)-1-((R)-aziridine-2-carbonyl)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-((R)-1-tritylaziridine-2-carbonyl)piperazin-2-yl)acetonitrile (79 mg, 93.6 μmol, 1 equiv) in DCM (0.5 mL) was added TFA (143 μL, 1.87 mmol, 20 equiv). The resulting mixture was stirred for 10 min then quenched with MeOH (1 mL) followed by NEt₃ (260 μL, 1.87 mmol, 20 equiv) and concentrated under reduced pressure. The residue was purified by reverse phase chromatography (10→60% MeCN/H₂O, 0.4% NH₄OH) then repurified by reverse phase chromatography (10→60% MeCN/H₂O, 0.4% NH₄OH) to afford 2-((S)-1-((R)-aziridine-2-carbonyl)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (9.5 mg, 17% yield over 2 steps) as white solid. LCMS (ESI) m/z: [M+H] calcd for C₃₂H₃₈ClN₈O₂: 601.28; found 601.8. ¹H NMR (500 MHz, Methanol-d₄) δ 7.84 (d, J=8.1 Hz, 1H), 7.70 (dd, J=8.2, 3.1 Hz, 1H), 7.58-7.47 (m, 2H), 7.43-7.31 (m, 2H), 5.06-4.93 (m, 1H), 4.62-4.48 (m, 1H), 4.35 (dt, J=18.1, 9.5 Hz, 4H), 4.27-4.04 (m, 2H), 3.81-3.68 (m, 2H), 3.67-3.41 (m, 2H), 3.29-3.17 (m, 3H), 3.17-3.01 (m, 3H), 3.00-2.84 (m, 1H), 2.82-2.63 (m, 2H), 2.52 (s, 3H), 2.37 (dd, J=9.0, 3.7 Hz, 1H), 2.11 (dq, J=16.5, 8.2 Hz, 1H), 1.94-1.79 (m, 4H), 1.79-1.64 (m, 1H).

### Example 113—Synthesis of 2-((S)-1-((S)-aziridine-2-carbonyl)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

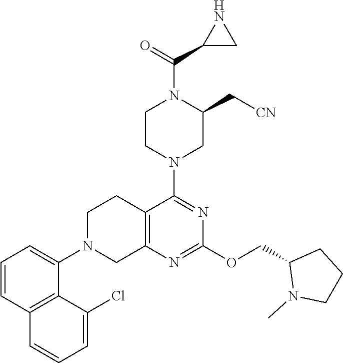 [SMILES: CN1CCC[C@H]1COc1nc2c(c(N3CCN(C(=O)[C@@H]4CN4)[C@@H](CC#N)C3)n1)CCN(c1cccc3cccc(Cl)c13)C2]

Synthesized according to the method of example 112, using (S)-1-tritylaziridine-2-carboxylic acid lithium salt in place of (R)-1-tritylaziridine-2-carboxylic acid lithium salt in step 6. LCMS (ESI) m/z: [M+H] calcd for C₃₂H₃₈ClN₈O₂: 601.28; found 601.8.

### Example 114—Synthesis of 2-((S))-1-((R)-aziridine-2-carbonyl)-4-(7-(3-hydroxynaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

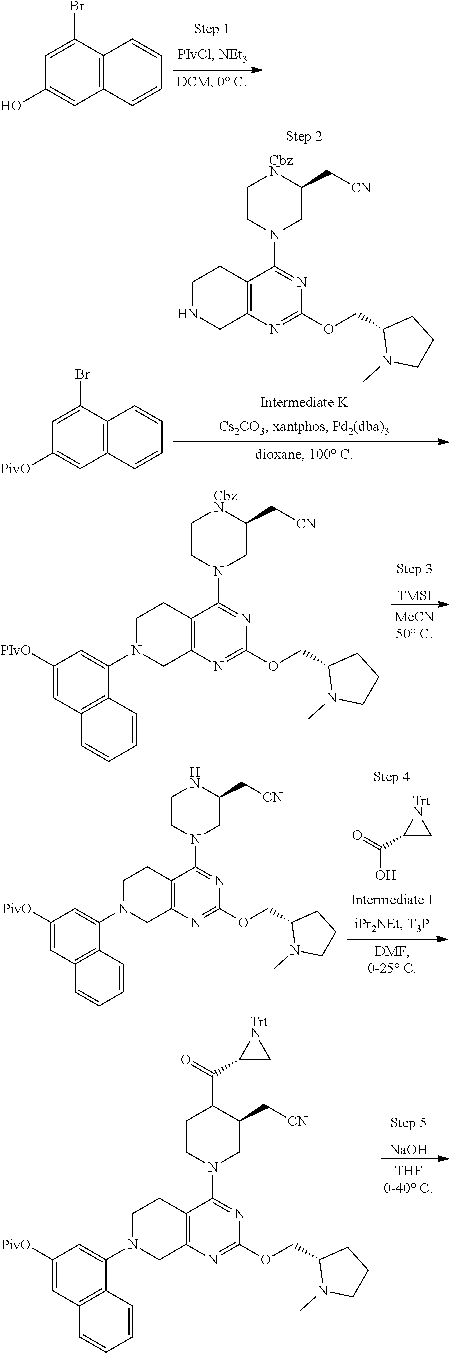

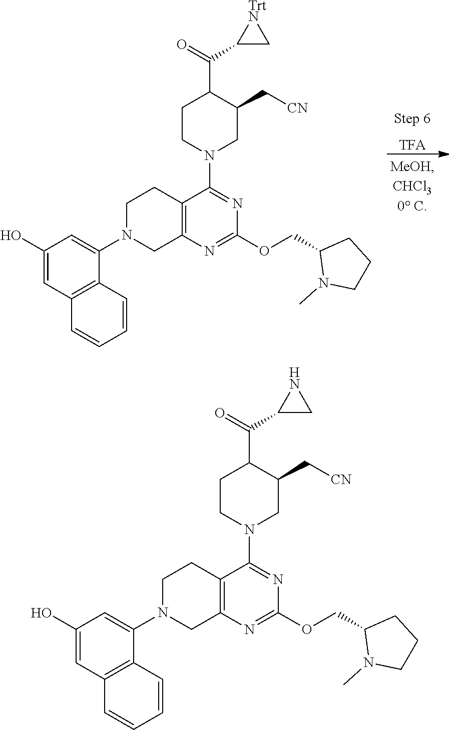

### Step 1: Synthesis of 4-bromonaphthalen-2-yl pivalate

To a solution of 4-bromonaphthalen-2-ol (1.0 g, 4.5 mmol, 1 equiv) in DCM (10 mL) at 0° C. was added NEt₃ (1.25 mL, 9.0 mmol, 2 equiv) and PivCl (830 μL, 6.7 mmol, 1.5 equiv). After 10 min the reaction was quenched with H₂O (60 mL), extracted into EtOAc (3×30 mL), dried over Na₂SO₄, filtered, and concentrated under reduced pressure. The crude residue was purified by silica gel column chromatography (2→5% EtOAc/petroleum ether) to afford 4-bromonaphthalen-2-yl pivalate (1.28 g, 93% yield) as a yellow oil. ¹HNMR (400 MHz, CDCl₃) δ 8.22 (d, J=8.16 Hz, 1H), 7.76-7.83 (m, 1H), 7.51-7.62 (m, 4H), 1.41 (s, 9H).

### Step 2: Synthesis of benzyl (S)-2-(cyanomethyl)-4-(2-(((S)-1-methylpyrrolidin-2-yl)methoxy)-7-(3-(pivaloyloxy)naphthalen-1-yl)-5,6,7,8-tetrahydropyrido[3,4-d]pyrimidin-4-yl)piperazine-1-carboxylate

A mixture of 4-bromonaphthalen-2-yl pivalate (1.3 g, 4.15 mmol, 1.5 equiv), benzyl (S)-2-(cyanomethyl)-4-(2-(((S)-1-methylpyrrolidin-2-yl)methoxy)-5,6,7,8-tetrahydropyrido[3,4-d]pyrimidin-4-yl)piperazine-1-carboxylate (1.4 g, 2.8 mmol, 1 equiv), RuPhos (260 mg, 550 μmol, 0.2 equiv), Pd₂(dba)₃ (260 mg, 280 μmol, 0.1 equiv), and Cs₂CO₃ (2.3 g, 6.9 mmol, 2.5 equiv) in dioxane (15 mL) was heated to 100° C. After 1 h the reaction mixture was filtered and concentrated under reduced pressure. The crude residue was diluted with sat. aq. NaCl (60 mL), extracted into EtOAc (2×50 mL), dried over Na₂SO₄, filtered, and concentrated under reduced pressure. The crude residue was purified by silica gel column chromatography (0→5% MeOH/DCM) to afford benzyl (S)-2-(cyanomethyl)-4-(2-(((S)-1-methylpyrrolidin-2-yl)methoxy)-7-(3-(pivaloyloxy)naphthalen-1-yl)-5,6,7,8-tetrahydropyrido[3,4-d]pyrimidin-4-yl)piperazine-1-carboxylate (1.7 g, 84% yield) as a yellow solid. LCMS (ESI) m/z: [M+H] calcd for C₄₂H₅₀N₇O₅: 732.39; found 732.4. ¹HNMR (400 MHz, CDCl₃) δ 8.12-8.18 (m, 1H), 7.77-7.83 (m, 1H), 7.48 (ddd, J=7.72, 5.84, 1.43 Hz, 2H), 7.35-7.42 (m, 5H), 7.29 (d, J=1.98 Hz, 1H), 6.83 (d, J=1.98 Hz, 1H), 5.22 (s, 2H), 4.70 (br d, J=2.87 Hz, 1H), 4.35-4.44 (m, 1H), 4.26 (br d, J=5.29 Hz, 2H), 4.17-4.22 (m, 1H), 4.03-4.09 (m, 1H), 3.91-4.01 (m, 1H), 3.44-3.56 (m, 1H), 3.22-3.41 (m, 3H), 2.95-3.16 (m, 3H), 2.86 (br d, J=1.32 Hz, 2H), 2.49 (s, 3H), 2.29 (br d, J=7.72 Hz, 1H), 1.64-1.92 (m, 6H), 1.41 (s, 9H).

### Step 3: Synthesis of 4-(4-((S)-3-(cyanomethyl)piperazin-1-yl)-2-(((S)-1-methylpyrrolidin-2-yl)methoxy)-5,6-dihydropyrido[3,4-d]pyrimidin-7(6H)-yl)naphthalen-2-yl pivalate

To a solution of benzyl (S)-2-(cyanomethyl)-4-(2-(((S)-1-methylpyrrolidin-2-yl)methoxy)-7-(3-(pivaloyloxy)naphthalen-1-yl)-5,6,7,8-tetrahydropyrido[3,4-d]pyrimidin-4-yl)piperazine-1-carboxylate (1.0 g, 1.4 mmol, 1 equiv) in MeCN (10 mL) was added TMSI (930 μL, 6.8 mmol, 5 equiv). The reaction mixture was heated to 50° C. After 1 h the reaction was quenched with MeOH (20 mL). After 15 min of stirring the mixture was added into HCl (1 N, 60 mL) and extracted into EtOAc (3×30 mL). The aqueous layer was basified to pH 8-9 with NaOH (1 N), extracted into EtOAc (3×30 mL), washed with sat. aq. NaCl (40 mL), dried over Na₂SO₄, filtered, and concentrated under reduced pressure to afford 4-(4-((S)-3-(cyanomethyl)piperazin-1-yl)-2-(((S)-1-methylpyrrolidin-2-yl)methoxy)-5,6-dihydropyrido[3,4-d]pyrimidin-7(6H)-yl)naphthalen-2-yl pivalate (1.27 g) as a yellow solid, which was used without further purification. LCMS (ESI) m/z: [M+H] calcd for C₃₄H₄₄N₇O₃: 598.35; found 598.3. ¹HNMR (400 MHz, CDCl₃) δ 8.16 (d, J=7.72 Hz, 1H), 7.75-7.84 (m, 1H), 7.44-7.54 (m, 2H), 7.29 (d, J=1.76 Hz, 1H), 6.83 (d, J=1.98 Hz, 1H), 4.43 (br s, 1H), 4.25 (s, 2H), 4.17-4.22 (m, 1H), 14.03 (br d, J=12.79 Hz, 1H), 3.89 (br d, J=11.69 Hz, 1H), 3.24-3.47 (m, 3H), 2.99-3.20 (m, 4H), 2.82-2.97 (m, 3H), 2.66-2.77 (m, 1H), 2.56 (dd, J=6.39, 2.87 Hz, 2H), 2.52 (br s, 3H), 2.26-2.40 (m, 1H), 1.71-1.95 (m, 4H), 1.41 (s, 9H).

### Step 4: Synthesis of 4-(4-((S)-3-(cyanomethyl)-4-((R)-1-tritylaziridine-2-carbonyl)piperazin-1-yl)-2-(((S)-1-methylpyrrolidin-2-yl)methoxy)-5,8-dihydropyrido[3,4-d]pyrimidin-7(6H)-yl)naphthalen-2-yl pivalate

To a solution of 4-(4-((S)-3-(cyanomethyl)piperazin-1-yl)-2-(((S)-1-methylpyrrolidin-2-yl)methoxy)-5,6-dihydropyrido[3,4-d]pyrimidin-7(6H)-yl)naphthalen-2-yl pivalate (570 mg, 950 μmol, 1 equiv) and (R)-1-tritylaziridine-2-carboxylic acid (940 mg, 2.9 mmol, 3 equiv) in DMF (6 mL) at 0° C. was added N,N-diisopropylethylamine (1.66 mL, 9.5 mmol, 10 equiv) and T₃P (1.70 mL, 2.9 mmol, 50% purity, 3 equiv). The reaction mixture was warmed to room temperature. After 1 h the reaction was quenched with H₂O (60 mL), extracted into EtOAc (3×30 mL), then the combined organic phase was washed with sat. aq. NaCl (2×30 mL), dried over Na₂SO₄, filtered, and concentrated under reduced pressure. The crude residue was purified by silica gel column chromatography (0→5% MeOH/DCM to afford 4-(4-((S)-3-(cyanomethyl)-4-((R)-1-tritylaziridine-2-carbonyl)piperazin-1-yl)-2-(((S)-1-methylpyrrolidin-2-yl)methoxy)-5,8-dihydropyrido[3,4-d]pyrimidin-7(6H)-yl)naphthalen-2-yl pivalate (1.14 g) as a yellow solid, which was used without further purification. LCMS (ESI) m/z: [M+H] calcd for C₅₆H₆₁N₈O₄: 909.48; found 909.5. ¹HNMR (400 MHz, CDCl₃) δ 7.93 (br d, J=7.72 Hz, 1H), 7.55-7.61 (m, 1H), 7.33 (br d, J=7.50 Hz, 5H), 7.06-7.12 (m, 7H), 6.93-6.99 (m, 6H), 6.61 (d, J=1.98 Hz, 1H), 4.89 (br d, J=2.87 Hz, 1H), 4.28 (br dd, J=10.69, 5.62 Hz, 1H), 3.95-4.11 (m, 4H), 3.54-3.63 (m, 1H), 3.37-3.46 (m, 1H), 3.15-3.32 (m, 3H), 3.06 (br s, 2H), 2.55-2.84 (m, 7H), 2.32-2.39 (m, 3H), 2.24 (br s, 2H), 1.52-1.78 (m, 5H), 1.19 (s, 9H).

### Step 5: Synthesis of 2-((S)-4-(7-(3-hydroxynaphthalen-1-yl)-2-(((S)-1-methylpyrrolidin-2-yl)methoxy)-5,6,7,8-tetrahydropyrido[3,4-d]pyrimidin-4-yl)-1-((R)-1-tritylaziridine-2-carbonyl)piperazin-2-yl)acetonitrile

To a solution of 4-(4-((S)-3-(cyanomethyl)-4-((R)-1-tritylaziridine-2-carbonyl)piperazin-1-yl)-2-(((S)-1-methylpyrrolidin-2-yl)methoxy)-5,8-dihydropyrido[3,4-d]pyrimidin-7(6H)-yl)naphthalen-2-yl pivalate (1.0 g, 1.1 mmol, 1 equiv) in THF (10 mL) at 0° C. was added NaOH (1.2 mL, 5 N, 5.5 equiv). The reaction mixture was heated to 40° C. After 16 h the reaction mixture was quenched with H₂O (60 mL), adjusted to pH 7 by 20% aq. formic acid, extracted into EtOAc (3×30 mL), dried over Na₂SO₄, filtered, and concentrated under reduced pressure to afford 2-((S)-4-(7-(3-hydroxynaphthalen-1-yl)-2-(((S)-1-methylpyrrolidin-2-yl)methoxy)-5,6,7,8-tetrahydropyrido[3,4-d]pyrimidin-4-yl)-1-((R)-1-tritylaziridine-2-carbonyl)piperazin-2-yl)acetonitrile (640 mg) as a yellow solid, which was used without further purification. LCMS (ESI) m/z: [M+H] calcd for C₅₁H₅₃N₈O₃: 825.42; found 825.4.

### Step 6: Synthesis of 2-((S)-1-((R)-aziridine-2-carbonyl)-4-(7-(3-hydroxynaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of 2-((S)-4-(7-(3-hydroxynaphthalen-1-yl)-2-(((S)-1-methylpyrrolidin-2-yl)methoxy)-5,6,7,8-tetrahydropyrido[3,4-d]pyrimidin-4-yl)-1-((R)-1-tritylaziridine-2-carbonyl)piperazin-2-yl)acetonitrile (400 mg, 490 μmol, 1 equiv) in CHCl₃ (2 mL) and MeOH (2 mL) at 0° C. was added TFA (540 μL, 7.3 mmol, 15 equiv). After 30 min the reaction mixture was quenched with 0° C. sat. aq. NaHCO₃ (60 mL), extracted into EtOAc (3×30 mL), dried over Na₂SO₄, filtered, and concentrated under reduced pressure. The crude residue was purified by reverse phase chromatography (25-55% MeCN/H₂O, 10 mM NH₄HCO₃) to afford 2-((S)-1-((R)-aziridine-2-carbonyl)-4-(7-(3-hydroxynaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (161 mg, 55% yield) as a yellow solid. LCMS (ESI) m/z: [M+H] calcd for C₃₂H₃₉N₈O₃: 583.31; found 583.4. ¹H NMR (400 MHz, CDCl₃) δ 7.94-8.02 (m, 1H), 7.60-7.68 (m, 1H), 7.38-7.50 (m, 1H), 7.28-7.34 (m, 1H), 6.88 (d, J=0.88 Hz, 1H), 6.53-6.63 (m, 1H), 4.80-4.95 (m, 1H), 4.46-4.64 (m, 1H), 3.80-4.37 (m, 5H), 3.08-3.51 (m, 4H), 2.92-3.06 (m, 1H), 2.31-2.90 (m, 10H), 2.05-2.19 (m, 1H), 1.68-2.02 (m, 7H).

### Example 115—Synthesis of 2-((S)-1-((S)-aziridine-2-carbonyl)-4-(7-(3-hydroxynaphthalen-1-yl)-2-(((S)-1-methylpyrrolidin-2-yl)ethoxy)-5,6,7,8-tetrahydropyrido[3,4-d]pyrimidin-4-yl)piperazin-2-yl)acetonitrile

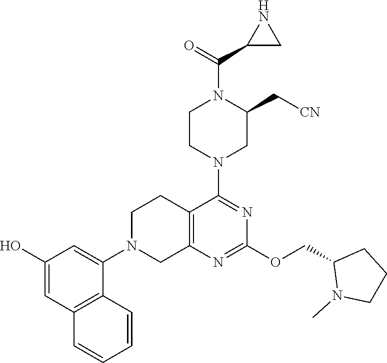 [SMILES: CN1CCC[C@H]1COc1nc2c(c(N3CCN(C(=O)[C@@H]4CN4)[C@@H](CC#N)C3)n1)CCN(c1cc(O)cc3ccccc13)C2]

Synthesized according to the method of example 114, using (S)-1-tritylaziridine-2-carboxylic acid in place of (R)-1-tritylaziridine-2-carboxylic acid in step 4. LCMS (ESI) m/z: [M+H] calcd for C₃₂H₃₉N₈O₃: 583.31; found 583.4.

### Example 116—Synthesis of 2-((S)-1-((R)-aziridine-2-carbonyl)-4-(2-(((S)-1-(but-3-yn-1-yl)pyrrolidin-2-yl)methoxy)-7-(8-methylnaphthalen-1-yl)-6,6,7,8-tetrahydropyrldo[3,4-d]pyrimidin-4-yl)piperazin-2-yl)acetonitrile

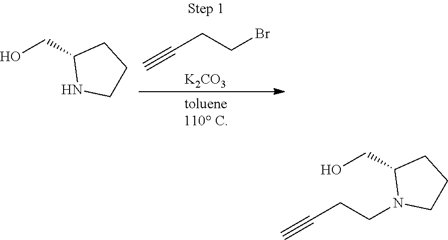

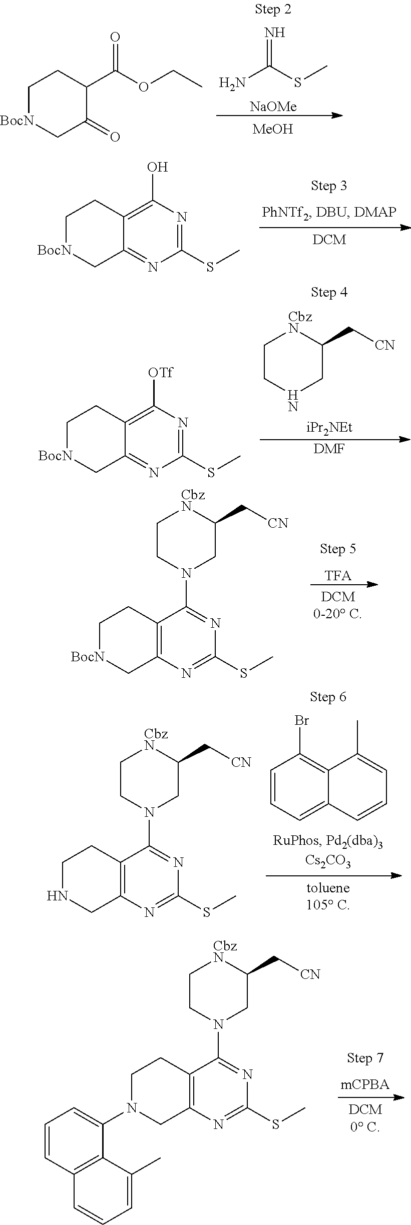

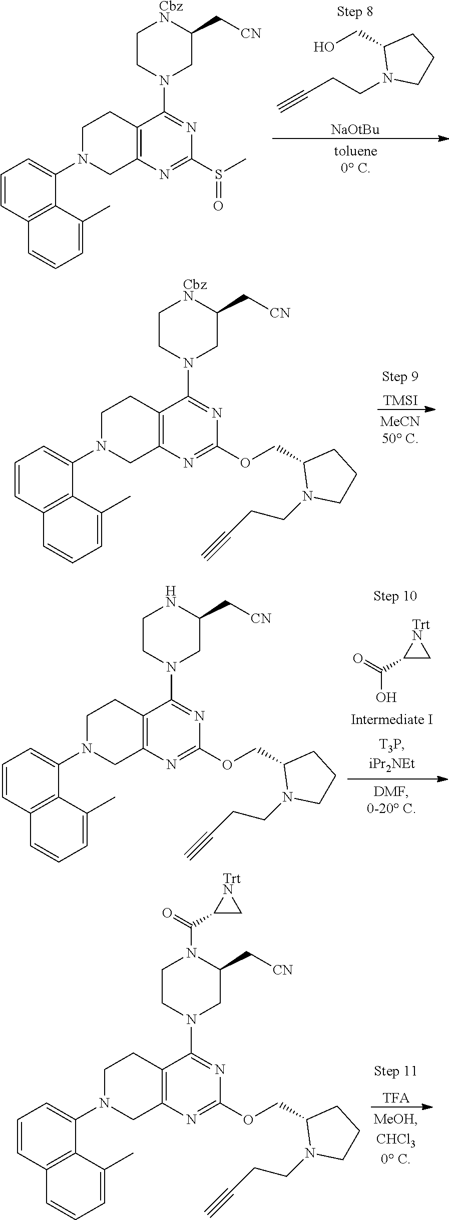

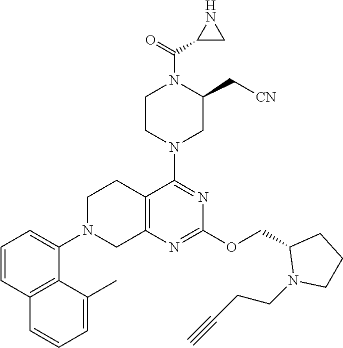 [SMILES: C#CCCN1CCC[C@H]1COc1nc2c(c(N3CCN(C(=O)[C@H]4CN4)[C@@H](CC#N)C3)n1)CCN(c1cccc3cccc(C)c13)C2]

### Step 1: Synthesis of (S)-(1-(but-3-yn-1-yl)pyrrolidin-2-yl)methanol

To a mixture of (S)-pyrrolidin-2-ylmethanol (9.6 mL, 99 mmol, 1 equiv) and K₂CO₃ (14 g, 100 mmol, 1.01 equiv) in toluene (100 mL) was added 4-bromobut-1-yne (11 mL, 120 mmol, 1.2 equiv). The resulting reaction mixture was heated to 110° C. After 16 h the mixture was quenched with HCl (50 mL, 2 N), washed with MTBE (50 mL), adjusted to pH 8-9 with sat. aq. NH₄OH, extracted into DCM (4×20 mL), washed with sat. aq. NaCl (3×20 mL), dried over Na₂SO₄, filtered, and concentrated under reduced pressure to afford (S)-(1-(but-3-yn-1-yl)pyrrolidin-2-yl)methanol (9.48 g, 63% yield) as yellow oil. ¹H NMR (400 MHz, CDCl₃) δ 3.69-3.55 (m, 1H), 3.37 (d, J=10.58 Hz, 1H), 3.25-3.11 (m, 1H), 2.91 (dt, J=12.07, 7.86 Hz, 1H), 2.80 (s, 1H), 2.65 (dd, J=5.40, 2.98 Hz, 1H), 2.51 (dt, J=12.13, 6.06 Hz, 1H), 2.41-2.22 (m, 3H), 2.04-1.92 (m, 1H), 1.90-1.82 (m, 1H), 1.80-1.59 (m, 3H).

### Step 2: Synthesis of tert-butyl 4-hydroxy-2-(methylthio)-5,8-dihydropyrido[3,4-d]pyrimidine-7(6H)-carboxylate

To a solution of 1-(tert-butyl) 4-ethyl 3-oxopiperidine-1,4-dicarboxylate (50 g, 180 mmol, 1 equiv) in NaOMe (50 mL, 920 mmol, 5 equiv, 56 wt % in MeOH) was added methyl carbamimidothioate (92 g, 330 mmol, 1.8 equiv). After 6 h the reaction mixture was quenched with HCl (2 N) to pH 5, concentrated under reduced pressure, suspended in EtOAc/H₂O (1.0 L, 1:1), and stirred. After 10 min the mixture was filtered, and the filter cake was washed with petroleum ether (100 mL) then dried under reduced pressure to afford tert-butyl 4-hydroxy-2-(methylthio)-5,8-dihydropyrido[3,4-d]pyrimidine-7(6H)-carboxylate (40.1 g, 73% yield) as white solid. ¹H NMR (400 MHz, CDCl₃) δ 4.34 (s, 2H), 3.60 (s, 2H), 2.64-2.43 (m, 5H), 1.50 (s, 9H).

### Step 3: Synthesis of tert-butyl 2-(methylthio)-4-(((trifluoromethyl)sulfonyl)oxy)-5,8-dihydropyrido[3,4-d]pyrimidine-7(6H)-carboxylate

To a solution of tert-butyl 4-hydroxy-2-(methylthio)-5,8-dihydropyrido[3,4-d]pyrimidine-7(6H)-carboxylate (15 g, 50 mmol, 1 equiv) in DCM (150 mL) was added PhNTf₂ (27 g, 76 mmol, 1.5 equiv), DBU (7.6 mL, 50 mmol, 1 equiv), and DMAP (120 mg, 1.01 mmol, 0.02 equiv) sequentially. After 1 h the reaction was quenched with H₂O (100 mL), extracted into DCM (3×50 mL), then the combined organic phase was washed with sat. aq. NaCl (2×30 mL), dried over Na₂SO₄, filtered, and concentrated under reduced pressure to afford tert-butyl 2-(methylthio)-4-(((trifluoromethyl)sulfonyl)oxy)-5,8-dihydropyrido[3,4-d]pyrimidine-7(6H)-carboxylate (50.25 g, crude) as yellow solid, which was used without further purification.

### Step 4: Synthesis of tert-butyl (S)-4-(4-((benzyloxy)carbonyl)-3-(cyanomethyl)piperazin-1-yl)-2-(methylthio)-5,8-dihydropyrido[3,4-d]pyrimidine-7(6H)-carboxylate

To a solution of tert-butyl 2-(methylthio)-4-(((trifluoromethylsulfonyloxy)-5,8-dihydropyrido[3,4-d]pyrimidine-7(6H)-carboxylate (20.2 g, 47.1 mmol, 43% purity, 1 equiv) and benzyl (S)-2-(cyanomethyl)piperazine-1-carboxylate.3HCl (20.0 g, 54.1 mmol, 1.15 equiv) in DMF (200 mL) was added N,N-diisopropylethylamine (41.0 mL, 235 mmol, 5 equiv). After 1 h the reaction was quenched with H₂O (600 mL), extracted into EtOAc (3×100 mL), then the combined organic phase was washed with sat. aq. NaCl (3×100 mL), dried over Na₂SO₄, filtered, and concentrated under reduced pressure. The crude residue was purified by silica gel column chromatography (0→33% EtOAc/petroleum ether) to afford tert-butyl (S)-4-(4-((benzyloxy)carbonyl)-3-(cyanomethyl)piperazin-1-yl)-2-(methylthio)-5,8-dihydropyrido[3,4-d]pyrimidine-7(6H)-carboxylate (21.5 g, 85% yield) as yellow oil. ¹H NMR (400 MHz, CDCl₃) δ 7.44-7.28 (m, 5H), 5.24-5.14 (m, 2H), 4.73-4.57 (m, 2H), 4.38 (d, J=18.96 Hz, 1H), 4.04-3.73 (m, 3H), 3.30 (d, J=11.03 Hz, 3H), 3.00 (td, J=12.46, 3.53 Hz, 1H), 2.86-2.56 (m, 4H), 2.51 (s, 3H), 1.49 (s, 9H).

### Step 5: Synthesis of benzyl (S)-2-(cyanomethyl)-4-(2-(methylthio)-5,6,7,8-tetrahydropyrido[3,4-d]pyrimidin-4-yl)piperazine-1-carboxylate

To a solution of tert-butyl (S)-4-(4-((benzyloxy)carbonyl)-3-(cyanomethyl)piperazin-1-yl)-2-(methylthio)-5,8-dihydropyrido[3,4-d]pyrimidine-7(6H)-carboxylate (18.0 g, 33.4 mmol, 1 equiv) in DCM (60 mL) at 0° C. was added TFA (61.9 mL, 835 mmol, 25 equiv). The reaction mixture was warmed to room temperature. After 1 h the mixture was concentrated under reduced pressure, added into sat. aq. NaHCO₃ (200 mL) at 0° C., extracted into DCM (3×30 mL), then the combined organic phase was washed with sat. aq. NaCl (3×20 mL), dried over Na₂SO₄, filtered, and concentrated under reduced pressure to afford benzyl (S)-2-(cyanomethyl)-4-(2-(methylthio)-5,6,7,8-tetrahydropyrido[3,4-d]pyrimidin-4-yl)piperazine-1-carboxylate (17.72 g, crude) as yellow solid, which was used without further purification. LCMS (ESI) m/z: [M+H] calcd for C₂₂H₂₇N₆O₂S: 439.19; found 439.2. ¹H NMR (400 MHz, CDCl₃) δ 7.42-7.29 (m, 5H), 5.22-5.13 (m, 2H), 4.72-4.59 (m, 1H), 4.26-4.09 (m, 4H), 3.99 (d, J=13.45 Hz, 1H), 3.88-3.75 (m, 1H), 3.48-3.17 (m, 4H), 3.13-2.98 (m, 1H), 2.96-2.75 (m, 3H), 2.73-2.60 (m, 1H), 2.51-2.46 (m, 3H).

### Step 6: Synthesis of benzyl (S)-2-(cyanomethyl)-4-(7-(8-methylnaphthalen-1-yl)-2-(methylthio)-5,6,7,8-tetrahydropyrido[3,4-d]pyrimidin-4-yl)piperazine-1-carboxylate

A solution of benzyl (S)-2-(cyanomethyl)-4-(2-(methylthio)-5,6,7,8-tetrahydropyrido[3,4-d]pyrimidin-4-yl)piperazine-1-carboxylate (15.7 g, 35.9 mmol, 1 equiv), 1-bromo-8-methyl-naphthalene (15.9 g, 71.7 mmol, 2 equiv), Cs₂CO₃ (29.2 g, 89.6 mmol, 2.5 equiv), RuPhos (3.35 g, 7.17 mmol, 0.2 equiv), and Pd₂(dba)₃ (3.28 g, 3.58 mmol, 0.1 equiv) in toluene (160 mL) was heated to 105° C. After 12 h the mixture was filtered to remove solids, added to H₂O (300 mL), and the aqueous layer was extracted with EtOAc (2×50 mL). The combined organic phase was washed with sat. aq. NaCl (3×30 mL), dried over Na₂SO₄, filtered, and concentrated under reduced pressure. The crude residue was purified by silica gel column chromatography (0→50% EtOAc/petroleum ether) to afford benzyl (S)-2-(cyanomethyl)-4-(7-(8-methylnaphthalen-1-yl)-2-(methylthio)-5,6,7,8-tetrahydropyrido[3,4-d]pyrimidin-4-yl)piperazine-1-carboxylate (6.63 g, 32% yield) as yellow solid. LCMS (ESI) m/z: [M+H] calcd for C₃₃H₃₅N₆O₂S: 579.25; found 579.3; ¹H NMR (400 MHz, CDCl₃) δ 7.72-7.62 (m, 2H), 7.42-7.34 (m, 7H), 7.25-7.14 (m, 2H), 5.21 (s, 2H), 4.69 (s, 1H), 4.31-4.19 (m, 1H), 4.00-3.74 (m, 3H), 3.56-3.39 (m, 2H), 3.25-3.09 (m, 3H), 3.07-2.94 (m, 2H), 2.91 (s, 3H), 2.80-2.58 (m, 3H), 2.50 (d, J=4.77 Hz, 3H).

### Step 7: Synthesis of benzyl (2S)-2-(cyanomethyl)-4-(7-(8-methylnaphthalen-1-yl)-2-(methylsulfinyl)-5,6,7,8-tetrahydropyrido[3,4-d]pyrimidin-4-yl)piperazine-1-carboxylate

To a solution of benzyl (S)-2-(cyanomethyl)-4-(7-(8-methylnaphthalen-1-yl)-2-(methylthio)-5,6,7,8-tetrahydropyrido[3,4-d]pyrimidin-4-yl)piperazine-1-carboxylate (7.66 g, 13.2 mmol, 1 equiv) in DCM (80 mL) at 0° C. was added mCPBA (3.22 g, 15.9 mmol, 85% purity, 1.2 equiv) portion wise. After 2 h the reaction mixture was quenched with sat. aq. Na₂SO₃, extracted into DCM (3×40 mL), then the combined organic phase was washed with sat. aq. NaCl (2×30 mL), dried over Na₂SO₄, filtered, and concentrated under reduced pressure. The crude residue was purified by silica gel column chromatography (0→100% EtOAc/petroleum ether) to afford benzyl (2S)-2-(cyanomethyl)-4-(7-(8-methylnaphthalen-1-yl)-2-(methylsulfinyl-5,6,7,8-tetrahydropyrido[3,4-d]pyrimidin-4-yl)piperazine-1-carboxylate (5.25 g, 67% yield) as yellow solid. LCMS (ESI) m/z: [M+H] calcd for C₃₃H₃₅N₆O₃S: 595.25; found 595.3. ¹H NMR (400 MHz, CDCl₃) δ 7.75-7.62 (m, 2H), 7.46-7.32 (m, 7H), 7.26-7.10 (m, 2H), 5.21 (s, 2H), 4.68 (s, 1H), 4.51-4.22 (m, 2H), 4.11-3.89 (m, 2H), 3.65-3.50 (m, 2H), 3.35-3.07 (m, 4H), 2.94-2.89 (m, 6H), 2.82-2.58 (m, 3H).

### Step 8 Synthesis of benzyl (S)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)-2-(cyanomethyl)piperazine-1-carboxylate

To a solution of benzyl (2S)-2-(cyanomethyl)-4-(7-(8-methylnaphthalen-1-yl)-2-(methylsulfinyl)-5,6,7,8-tetrahydropyrido[3,4-d]pyrimidin-4-yl)piperazine-1-carboxylate (5.25 g, 8.83 mmol, 1 equiv) and (S)-(1-(but-3-yn-1-yl)pyrrolidin-2-yl) methanol (2.71 g, 17.7 mmol, 2 equiv) in toluene (50 mL) at 0° C. was added NaOtBu (1.70 g, 17.7 mmol, 2 equiv). After 20 min the mixture quenched with H₂O (60 mL), extracted into EtOAc (2×30 mL), then the combined organic phase was washed with sat. aq. NaCl (3×20 mL), dried over Na₂SO₄, filtered, and concentrated under reduced pressure. The crude residue was purified by silica gel column chromatography (0→50% EtOAc/petroleum ether) to afford benzyl (S)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)-2-(cyanomethyl)piperazine-1-carboxylate (3.82 g, 63% yield) as yellow solid. LCMS (ESI) m/z: [M+H] calcd for C₄₁H₄₆N₇O₃: 684.37; found 684.4. ¹H NMR (400 MHz, CDCl₃) δ 7.90-7.68 (m, 1H), 7.71-7.59 (m, 1H), 7.44-7.34 (m, 6H), 7.26-7.18 (m, 2H), 5.21 (s, 2H), 4.69 (s, 1H), 4.44-3.69 (m, 8H), 3.57-3.33 (m, 2H), 3.26-3.03 (m, 5H), 2.92 (s, 5H), 2.79-2.60 (m, 3H), 2.46-2.25 (m, 3H), 2.02-1.62 (m, 6H).

### Step 9: Synthesis of 2-((S)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)piperazin-2-yl)acetonitrile

To a solution of benzyl (S)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)-2-(cyanomethyl)piperazine-1-carboxylate (3.82 g, 5.59 mmol, 1 equiv) in MeCN (40 mL) was added TMSI (3.04 mL, 22.3 mmol, 4 equiv), and the reaction mixture was heated to 50° C. After 2 h the reaction was cooled to room temperature, quenched with MeOH (20 mL), and stirred. After 15 min the mixture was poured into HCl (1 N, 100 mL), which was kept between 0-15° C., extracted into EtOAc (3×20 mL), then the aqueous layer was basified to pH 8-9 with NaOH (1 N), while being kept between 0-10° C., and then extracted with EtOAc (3×30 mL). The combined organic phase was washed with sat. aq. NaCl (3×20 mL), dried over Na₂SO₄, filtered, and concentrated under reduced pressure. The crude residue was purified by silica gel column chromatography (0→50% EtOAc/petroleum ether) to afford 2-((S)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)piperazin-2-yl)acetonitrile (1.04 g, 30% yield) as yellow solid. LCMS (ESI) m/z: [M+H] calcd for C₃₃H₄₀N₇O: 550.33; found 550.3. ¹H NMR (400 MHz, CDCl₃) δ 7.65-7.54 (m, 2H), 7.27 (m, 2H), 7.17-7.12 (m, 1H), 4.31 (s, 1H), 4.15 (dd, J=18.18, 6.85 Hz, 1H), 4.01 (m, 1H), 3.87-3.63 (m, 3H), 3.46-3.24 (m, 1H), 3.16-3.00 (m, 5H), 2.94-2.74 (m, 7H), 2.65-2.46 (m, 4H), 2.44-2.24 (m, 3H), 1.96-1.48 (m, 7H).

### Step 10: Synthesis of 2-((S)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)-1-((R)-1-tritylaziridine-2-carbonyl)piperazin-2-yl)acetonitrile

To a mixture of 2-((S)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)piperazin-2-yl)acetonitrile (100 mg, 180 μmol, 1 equiv) and (R)-1-tritylaziridine-2-carboxylic acid (420 mg, 1.27 mmol, 7 equiv) in DMF (1 mL) at 0° C. was added N,N-diisopropylethylamine (160 μL, 910 μmol, 5 equiv) followed by T₃P (216 μL, 360 μmol, 50% purity, 2 equiv). The mixture was warmed to room temperature. After 2 h the reaction was quenched with cold sat. aq. NH₄Cl, extracted into EtOAc (3×5 mL), then the combined organic phase was washed with sat. aq. NaCl (10 mL), dried over Na₂SO₄, filtered, and concentrated under reduced pressure. The crude residue was purified by silica gel column chromatography (0→70% EtOAc/petroleum ether) to afford 2-((S)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)-1-((R)-1-tritylaziridine-2-carbonyl)piperazin-2-yl)acetonitrile (220 mg, crude) as brown solid, which was used without further purification. ¹H NMR (400 MHz, CDCl₃) δ 7.63-7.73 (m, 1H), 7.52-7.62 (m, 4H), 7.38-7.51 (m, 7H), 7.27 (m, 15H), 5.12 (br s, 1H), 4.44 (br s, 1H), 4.21-4.38 (m, 1H), 3.73-4.05 (m, 2H), 3.39-3.71 (m, 2H), 3.00-3.35 (m, 4H), 2.93 (br s, 3H), 2.53-2.79 (m, 3H), 2.36-2.52 (m, 3H), 2.24 (s, 1H), 1.95-2.04 (m, 2H), 1.70-1.93 (m, 3H), 1.39-1.57 (m, 2H).

### Step 11: Synthesis of 2-((S)-1-((R)-aziridine-2-carbonyl)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)piperazin-2-yl)acetonitrile

To a solution of 2-((S)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)-1-((R)-1-tritylaziridine-2-carbonyl)piperazin-2-yl)acetonitrile (170 mg, 200 μmol, 1 equiv) in MeOH (800 μL) and CHCl₃ (800 μL) at 0° C. was added TFA (290 μL, 4.0 mmol, 20 equiv). After 30 min the mixture was quenched with cold sat. aq. NaHCO₃, extracted into DCM (3×5 mL), then the combined organic phase was washed with sat. aq. NaCl (20 mL), dried over Na₂SO₄, filtered, and concentrated under reduced pressure. The crude residue was purified by reverse phase chromatography (40→70% MeCN/H₂O, 10 mM NH₄HCO₃) to afford 2-((S)-1-((R)-aziridine-2-carbonyl)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)piperazin-2-yl)acetonitrile (25.1 mg, 13% yield) as white solid. LCMS (ESI) m/z: [M+H] calcd for C₃₆H₄₃N₈O₂: 619.35; found 619.4. ¹H NMR (400 MHz, CDCl₃) δ 7.59-7.76 (m, 2H), 7.38-7.45 (m, 1H), 7.35 (t, J=7.64 Hz, 1H), 7.27-7.32 (m, 1H), 7.18-7.26 (m, 1H), 4.56-5.08 (m, 1H), 4.36 (br d, J=10.51 Hz, 1H), 4.19-4.32 (m, 1H), 4.02-4.18 (m, 3H), 3.85-4.01 (m, 1H), 3.68-3.84 (m, 1H), 3.36-3.59 (m, 2H), 2.98-3.33 (m, 5H), 2.93 (s, 4H), 2.54-2.89 (m, 4H), 2.24-2.46 (m, 3H), 1.71-2.08 (m, 6H), 1.59 (br s, 4H).

### Example 117—Synthesis of 2-((S)-1-((S)-aziridine-2-carbonyl)-4-(2-(((S)-1-(but-3-yn-1-yl)pyrrolidin-2-yl)methoxy)-7-(8-methylnaphthalen-1-yl)-5,6,7,8-tetrahydropyrido[3,4-d]pyrimidin-4-yl)piperazin-2-yl)acetonitrile

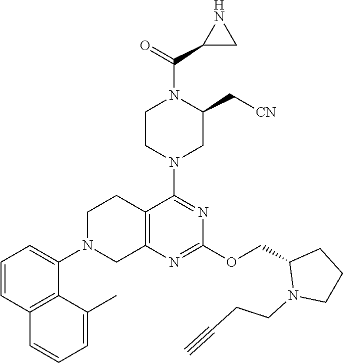 [SMILES: C#CCCN1CCC[C@H]1COc1nc2c(c(N3CCN(C(=O)[C@@H]4CN4)[C@@H](CC#N)C3)n1)CCN(c1cccc3cccc(C)c13)C2]

Synthesized according to the method of example 116, using (S)-1-tritylaziridine-2-carboxylic acid in place of (R)-1-tritylaziridine-2-carboxylic acid in step 10. LCMS (ESI) m/z: [M+H] calcd for C₃₆H₄₃N₈O₂: 619.35; found 619.4.

### Example 118—Synthesis of 2-((S)-1-(((R)-1-acetylaziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

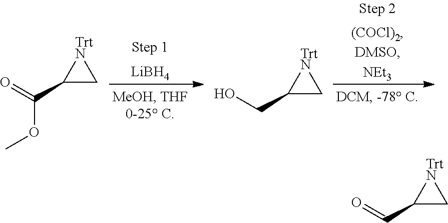

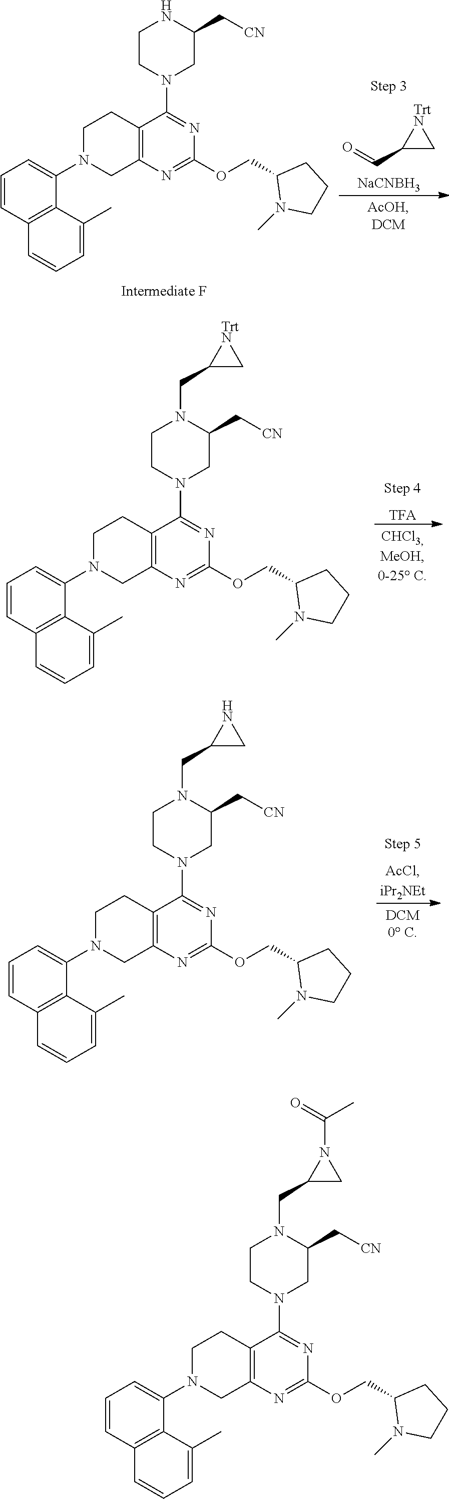

### Step 1: Synthesis of (S)-(1-tritylaziridin-2-yl)methanol

To a solution of methyl (S)-1-tritylaziridine-2-carboxylate (1.3 g, 3.8 mmol, 1 equiv) in THF (13 mL) at 0° C. was added LiBH₄ (412 mg, 18.9 mmol, 5 equiv) followed by the dropwise addition of MeOH (2.6 mL). The resulting mixture was warmed to room temperature. After 3 h the reaction was slowly quenched with H₂O (20 mL), extracted into EtOAc (3×10 mL), dried over Na₂SO₄, filtered, and concentrated under reduced pressure to afford crude (S)-(1-tritylaziridin-2-yl)methanol as white solid, which was used without further purification. ¹H NMR (400 MHz, CDCl₃) δ 1.11 (d, J=6.17 Hz, 1H), 1.56 (dq, J=6.28, 3.12 Hz, 1H), 1.85 (d, J=3.31 Hz, 1H), 2.20 (br s, 1H), 3.68 (m, 1H), 3.87 (m, 1H), 7.17-7.23 (m, 3H), 7.27 (s, 6H), 7.38-7.51 (m, 6H).

### Step 2: Synthesis of (S)-1-tritylaziridine-2-carbaldehyde

To a solution of (COCl)₂ (395 μL, 4.5 mmol, 1.2 equiv) in DCM (4 mL) at −78° C. was added a solution of DMSO (734 μL, 9.4 mmol, 2.5 equiv) in DCM (4.5 mL) dropwise. After 30 min a solution of (S)-(1-tritylaziridin-2-yl)methanol (1.5 g, 3.8 mmol, 1 equiv) in DCM (10 mL) was added dropwise to the reaction mixture. After 30 min NEt₃ (2.6 mL, 19 mmol, 5 equiv) was added. After 1 h the reaction was warmed to room temperature, quenched with H₂O (10 mL) and extracted into DCM. The combined organic phase was washed with sat. aq. NaCl, dried over Na₂SO₄, filtered, and concentrated under reduced pressure to afford (S)-1-tritylaziridine-2-carbaldehyde as white solid, which was used without further purification. ¹H NMR (400 MHz, CDCl₃) δ 1.54 (d, J=6.32 Hz, 1H), 1.94 (td, J=6.32, 2.50 Hz, 1H), 2.32 (d, J=2.03 Hz, 1H), 7.19-7.24 (m, 3H), 7.27 (s, 6H), 7.45 (d, J=7.39 Hz, 6H), 9.32 (d, J=6.44 Hz, 1H).

### Step 3: Synthesis of 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)-1-(((R)-1-tritylaziridin-2-yl)methyl)piperazin-2-yl)acetonitrile

Two separate reactions were run in parallel. For each reaction, to a suspension of 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (583 mg, 1.14 mmol, 1 equiv) and (S)-1-tritylaziridine-2-carbaldehyde (500 mg, 1.60 mmol, 1.4 equiv) in DCM (8 mL) was added AcOH (261 μL, 4.56 mmol, 4 equiv). After 10 min NaCNBH₃ (100 mg, 1.60 mmol, 1.4 equiv) was added. After 1 h the reaction was quenched slowly with H₂O (20 mL) and extracted into DCM (3×10 mL). The combined organic phase was washed with sat. aq. NaCl (30 mL), dried over Na₂SO₄, filtered, and concentrated under reduced pressure. The two separate crude residues were combined and purified by reverse phase chromatography (75→95% MeOH/H₂O, 9% MeCN, 0.05% NH₃OH), to afford 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)-1-(((R)-1-tritylaziridin-2-yl)methyl)piperazin-2-yl)acetonitrile (1.2 g, 65% yield) as a white solid. LCMS (ESI) m/z: [M+H] calcd for C₅₂H₅₇N₈O: 809.46; found 809.4. ¹H NMR (400 MHz, Methanol-d₄) δ 0.89 (br d, J=8.93 Hz, 1H), 1.21-1.33 (m, 2H), 1.43 (br d, J=2.93 Hz, 1H), 1.62-1.88 (m, 4H), 1.99-2.14 (m, 1H), 2.27-2.40 (m, 1H), 2.40-2.54 (m, 4H), 2.61 (br d, J=12.10 Hz, 1H), 2.67-2.78 (m, 2H), 2.79-2.96 (m, 4.5H), 2.97-3.09 (m, 2H), 3.10-3.23 (m, 2H), 3.32-3.38 (m, 1H), 3.42-3.60 (m, 3H), 3.61-3.79 (m, 2H), 3.85-3.95, (m, 0.5H), 4.08 (br dd, J=17.79, 7.64 Hz, 1H), 4.21-4.41 (m, 2H), 7.16-7.25 (m, 4H), 7.26-7.35 (m, 8H), 7.40 (td, J=7.67, 3.36 Hz, 1H), 7.51 (br d, J=7.95 Hz, 6H), 7.56-7.72 (m, 2H).

### Step 4: Synthesis of 2-((S)-1-(((S)-aziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of 2-((S)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)-1-(((R)-1-tritylaziridin-2-yl)methyl)piperazin-2-yl)acetonitrile (300 mg, 371 μmol, 1 equiv) in CHCl₃ (1.2 mL) and MeOH (1.8 mL) at 0° C. was added TFA (1.65 mL, 22 mmol, 60 equiv). After 1 h the reaction was warmed to room temperature. After 12 h the reaction mixture was concentrated under reduce pressure to remove excess TFA, dissolved in DCM (3 mL), then added dropwise sat. aq. NaHCO₃ (10 mL) at 0° C. and extracted into DCM (3×5 mL). The combined organic phase was washed with sat. aq. NaCl (10 mL), dried over Na₂SO₄, filtered, and concentrated under reduced pressure. The crude residue was purified by reverse phase chromatography (50→80% MeCN/H₂O, 10 mM NH₄HCO₃) to afford 2-((S)-1-(((S)-aziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (25 mg, 12% yield) as white solid. LCMS (ESI) m/z: [M+H] calcd for C₃₃H₄₃N₈O: 567.36; found 567.4. ¹H NMR (400 MHz, Methanol-d₄) δ 1.44 (br s, 1H), 1.62-1.74 (m, 1H), 1.74-1.89 (m, 3H), 2.00-2.13 (m, 1H), 2.18 (br s, 1H), 2.28-2.40 (m, 1H), 2.49 (d, J=2.57 Hz, 3H), 2.59-2.86 (m, 6.5H), 2.91 (s, 3H), 2.97-3.11 (m, 3H), 3.11-3.27 (m, 3H), 3.40-3.85 (m, 6H), 3.87-3.96 (m, 0.5H), 4.08 (br dd, J=17.73, 10.27 Hz, 1H), 4.25-4.43 (m, 2H), 7.18-7.36 (m, 3H), 7.40 (td, J=7.73, 4.22 Hz, 1H), 7.66 (br dd, J=17.18, 8.01 Hz, 2H).

### Step 5: Synthesis of 2-((S)-1-(((R)-1-acetylaziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a mixture of 2-((S)-1-((S)-aziridin-2-ylmethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (88 mg, 155 μmol, 1 equiv) in DCM (200 μL) at 0° C. was added N,N-diisopropylethylamine (135 μL, 776 μmol, 5 equiv) followed by the dropwise addition of AcCl (13.3 μL, 186 μmol, 1.2 equiv). After 5 min the reaction was added dropwise into H₂O (5 mL) at 0° C. then extracted into DCM (3×5 mL). The combined organic phase was washed with sat. aq. NaCl (3×20 mL), dried over Na₂SO₄, filtered, and concentrated under reduced pressure. The crude residue was purified by reverse phase chromatography (50→70% MeCN/H₂O, 10 mM NH₄HCO₃) to afford 2-((S)-1-(((R)-1-acetylaziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (40 mg, 20% yield) as a white solid. LCMS (ESI) m/z: [M+H] calcd for C₃₅H₄₅N₈O₂: 609.37; found 609.4. ¹H NMR (400 MHz, Methanol-d₄) δ 7.67 (dd, J=17.18, 8.01 Hz, 2H), 7.41 (td, J=7.73, 3.97 Hz, 1H), 7.27-7.37 (m, 2H), 7.21-7.27 (m, 1H), 4.28-4.42 (m, 2H), 4.09 (br dd, J=17.73, 10.03 Hz, 1H), 3.92 (br dd, J=13.20, 2.57 Hz, 1H), 3.56-3.66 (m, 1H), 3.42-3.56 (m, 2H), 3.41-3.86 (m, 3H), 3.12-3.27 (m, 3H), 3.01-3.12 (m, 2H), 2.95-3.01 (m, 1H), 2.92 (s, 3H), 2.63-2.86 (m, 7H), 2.50 (d, J=3.30 Hz, 3H), 2.45 (br d, J=4.77 Hz, 1H), 2.30-2.41 (m, 1H), 2.17 (s, 3H), 2.03-2.13 (m, 1H), 1.76-1.87 (m, 2H), 1.65-1.76 (m, 1H).

### Example 119—Synthesis of 2-((S)-1-(((S)-1-acetylaziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

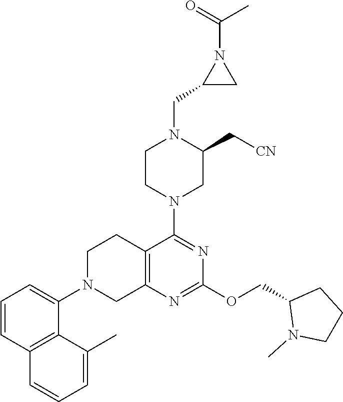 [SMILES: CC(=O)N1C[C@@H]1CN1CCN(c2nc(OC[C@@H]3CCCN3C)nc3c2CCN(c2cccc4cccc(C)c24)C3)C[C@@H]1CC#N]

Synthesized according to the method of example 118, using methyl (R)-1-tritylaziridine-2-carboxylate in place of methyl (S)-1-tritylaziridine-2-carboxylate in step 10. LCMS (ESI) m/z: [M+H] calcd for C₃₅H₄₅N₈O₂: 609.37; found 609.5.

### Example 120—Synthesis of ((R)-aziridin-2-yl)((8aS)-6-chloro-5-(2-fluoro-6-hydroxyphenyl)-8a,9,11,12-tetrahydropyrazino[2′,1′:3,4][1,4]oxazepino[5,6,7-de]quinazolin-10(8H)-yl)methanone

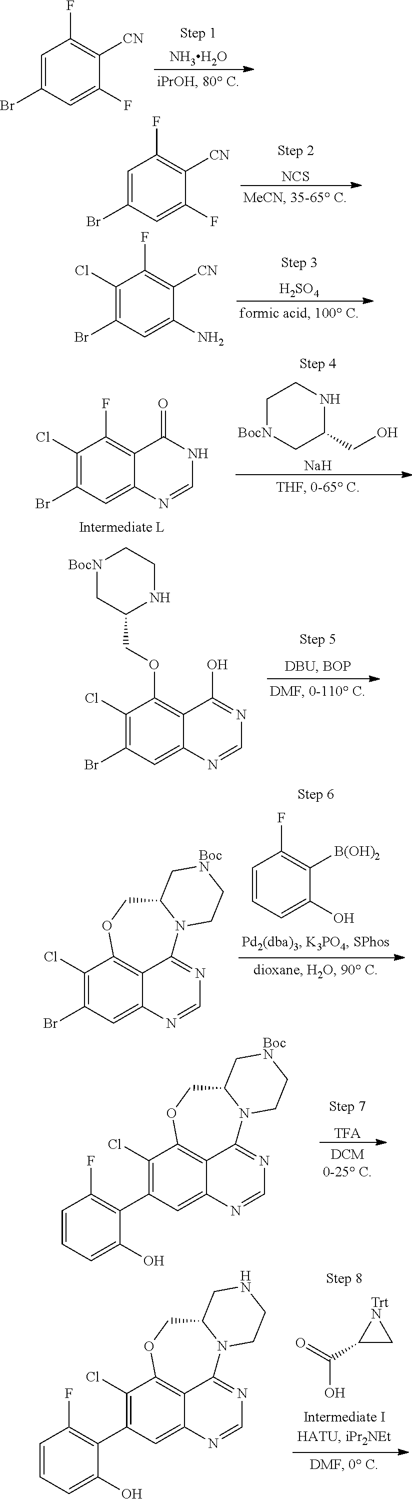

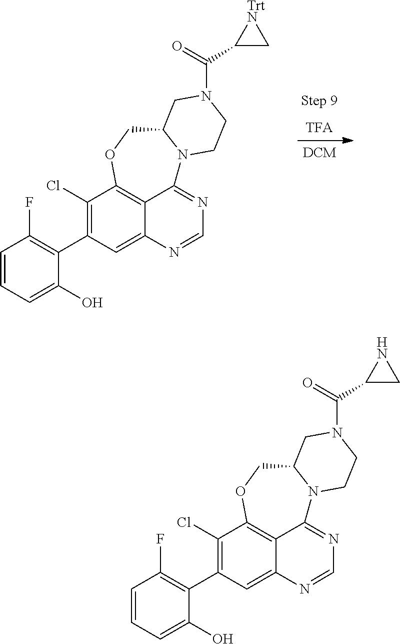

### Step 1: Synthesis of 2-amino-4-bromo-6-fluorobenzonitrile

Four separate reactions were run in parallel. For each reaction, to a solution of 4-bromo-2,6-difluorobenzonitrile (4.0 g, 18 mmol, 1 equiv) in iPrOH (40 mL) was added NH₃.H₂O (20 mL, 130 mmol, 25% w/w, 7 equiv). The resulting mixture was sealed and heated to 80° C. for 12 h then cooled to room temperature. The four separate reaction mixtures were combined and quenched with H₂O (640 mL), and stirred for 15 min. The resulting solids were filtered, washed with H₂O (100 mL), dissolved in toluene (3×10 mL) and concentrated under reduced pressure to afford 2-amino-4-bromo-6-fluorobenzonitrile (14.5 g, 92% yield) as a white solid. ¹H NMR (400 MHz, DMSO-d₆) δ 6.74 (br s, 2H), 6.78 (dd, J=9.26, 1.54 Hz, 1H), 6.82 (s, 1H).

### Step 2: Synthesis of 6-amino-4-bromo-3-chloro-2-fluorobenzonitrile

To a solution of 2-amino-4-bromo-8-fluorobenzonitrile (9.5 g, 44 mmol, 1 equiv) in MeCN (50 mL) at 35° C. was added NCS (5.9 g, 44 mmol, 1 equiv) portion wise and the resulting mixture was gradually heated to 65° C. After 24 h the reaction was quenched with H₂O (400 mL) and extracted into EtOAc (3×100 mL). The combined organic phase was washed with sat. aq. NaCl (80 mL), dried over Na₂SO₄, filtered, and concentrated under reduced pressure. The crude residue was triturated with 1:1 EtOAc/petroleum ether at 17° C. for 20 min, then filtered to afford crude product which was combined with crude product recrystallized from DCM (28 mL) and purified by silica gel column chromatography (0→10% EtOAc/petroleum ether) to afford 6-amino-4-bromo-3-chloro-2-fluorobenzonitrile (14 g, 37% yield) as white solid. ¹H NMR (400 MHz, DMSO-d₆) δ 6.84 (s, 2H), 7.02 (d, 1H).

### Step 3: Synthesis of 7-bromo-6-chloro-5-fluoroquinazolin-4(3H)-one

To a solution of 6-amino-4-bromo-3-chloro-2-fluorobenzonitrile (10 g, 40 mmol, 1 equiv) in formic acid (100 mL) was added H₂SO₄ (3.2 mL, 60 mmol, 1.5 equiv) and the resulting mixture was heated to 100° C. After 30 min the reaction mixture was cooled to room temperature, quenched with H₂O (200 mL), stirred for 10 min, then filtered. The filter cake was washed sequentially with 1:1 H₂O/iPrOH (100 mL), 1:1 IPrOH/MTBE (100 mL), and MTBE (100 mL), then triturated with EtOAc (100 mL) for 30 min, filtered, and concentrated under reduced pressure to afford 7-bromo-6-choro-5-fluoroquinazolin-4(3H)-one (8.8 g, 79% yield) as white solid. ¹H NMR (400 MHz, DMSO-d₆) δ 7.92 (d, J=1.76 Hz, 1H), 8.15 (s, 1H), 12.55 (br s, 1H).

### Step 4: Synthesis of tert-butyl (S)-3-(((7-bromo-6-chloro-4-hydroxyquinazolin-5-yl)oxy)methyl)piperazine-1-carboxylate

To a solution of tert-butyl (S)-3-(hydroxymethyl)piperazine-1-carboxylate (7.5 g, 35 mmol, 1.1 equiv) in THF (120 mL) at 0° C. was added NaH (3.8 g, 95 mmol, 60% purity, 3 equiv) portion wise. After 10 min the reaction was warmed to room temperature. After 30 min 7-bromo-6-chloro-5-fluoroquinazolin-4(3H)-one (8.8 g, 32 mmol, 1 equiv) was added to the mixture and the reaction was heated to 65° C. After 16 h the reaction was quenched with sat. aq. NH₄Cl (520 mL), extracted with EtOAc (4×200 mL), then the combined organic phase was washed with sat. aq. NaCl (300 mL), dried over Na₂SO₄, filtered, and concentrated under reduced pressure. The resulting residue was purified by silica gel column chromatography (9→17% MeOH/DCM) to afford tert-butyl (S)-3-(((7-bromo-8-chloro-4-hydroxyquinazolin-5-yl)oxy)methyl)piperazine-1-carboxylate (11.1 g, 66% yield) as yellow solid. ¹H NMR (400 MHz, DMSO-d₆) δ 8.10 (s, 1H), 7.84 (s, 1H), 5.74-5.77 (m, 1H), 4.06 (br d, J=11.69 Hz, 1H), 3.95 (br d, J=5.95 Hz, 2H), 3.76 (br d, J=11.69 Hz, 1H), 2.99 (br d, J=6.17 Hz, 1H), 2.91 (br d, J=11.91 Hz, 1H), 2.54-2.86 (m, 3H), 1.40 (s, 9H).

### Step 5: Synthesis of tert-butyl (S)-5-bromo-6-chloro-8a,9,11,12-tetrahydropyrazino[2′,1′:3,4][1,4]oxazepino[5,6,7-de]quinazoline-10(8H)-carboxylate

To a solution of tert-butyl (S)-3-(((7-bromo-8-chloro-4-hydroxyquinazolin-5-yl)oxy)methyl) piperazine-1-carboxylate (7.4 g, 15.6 mmol, 1 equiv) and BOP (18 g, 41 mmol, 2.6 equiv) in DMF (150 mL) at 0° C. was added DBU (11.8 mL, 78.1 mmol, 5 equiv) dropwise. After 10 min the reaction mixture was heated to 110° C. After 2 h the reaction mixture was quenched with H₂O (1.5 L), extracted with EtOAc (3×700 mL), then the combined organic phase was washed with sat. aq. NaCl (3×500 mL), dried over Na₂SO₄, filtered, and concentrated under reduced pressure. The crude residue was purified by silica gel column chromatography (44→100% EtOAc/petroleum ether) to afford tert-butyl (S)-5-bromo-8-chloro-8a,9,11,12-tetrahydropyrazino[2′,1′:3,4][1,4]oxazepino[5,6,7-de]quinazoline-10(8H)-carboxylate (7.0 g, 95% yield) as a yellow solid. ¹H NMR (400 MHz, DMSO-d₆) δ 8.53 (s, 1H), 7.80 (s, 1H), 4.79 (br d, J=13.11 Hz, 1H), 4.56-4.67 (m, 2H), 4.04-4.10 (m, 1H), 3.96-4.01 (m, 1H), 3.91 (br d, J=12.99 Hz, 1H), 3.15-3.26 (m, 1H), 3.06 (br s, 2H), 1.43 (s, 9H).

### Step 6: Synthesis of tert-butyl (8aS)-6-chloro-5-(2-fluoro-6-hydroxyphenyl)-8a,9,11,12-tetrahydropyrazino[2′,1′:3,4][1,4]oxazepino[5,6,7-de]quinazoline-10(8H)-carboxylate

To a solution tert-butyl (S)-5-bromo-8-chloro-8a,9,11,12-tetrahydropyrazino[2′,1′:3,4][1,4]oxazepino[5,6,7-de]quinazoline-10(8H)-carboxylate (10 g, 22 mmol, 1 equiv) and (2-fluoro-6-hydroxyphenyl)boronic acid (4.1 g, 26 mmol, 1.2 equiv) in dioxane (100 mL) at 15° C. was added SPhos (900 mg, 2.2 mmol, 0.1 equiv), Pd₂(dba)₃ (2.0 g, 2.2 mmol, 0.1 equiv), and a solution of K₃PO₄ (9.3 g, 44 mmol, 2 equiv) in H₂O (25 mL). The resulting mixture was heated to 90° C. After 2 h the reaction was cooled to room temperature, filtered to remove solids, added to H₂O (500 mL), and extracted into EtOAc (3×300 mL). The combined organic phase was washed with sat. aq. NaCl (200 mL), dried over Na₂SO₄, filtered, and concentrated under reduced pressure. The crude residue was purified by silica gel column chromatography (44-100% EtOAc/petroleum ether) to afford tert-butyl (8aS)-6-chloro-5-(2-fluoro-6-hydroxyphenyl)-8a,9,11,12-tetrahydropyrazino[2′,1′:3,4][1,4]oxazepino[5,6,7-de]quinazoline-10(8H)-carboxylate (9 g, 82% yield) as red solid. ¹H NMR (400 MHz, DMSO-d₆) δ 10.04 (br s, 1H), 8.54 (s, 1H), 7.33 (d, J=1.54 Hz, 1H), 7.24-7.32 (m, 1H), 6.81 (dd, J=8.27, 4.08 Hz, 1H), 6.75 (td, J=8.71, 2.43 Hz, 1H), 4.82 (br d, J=11.25 Hz, 1H), 4.58-4.69 (m, 2H), 4.04-4.12 (m, 1H), 3.89-4.01 (m, 2H), 2.96-3.27 (m, 3H), 1.44 (s, 9H).

### Step 7: Synthesis of 2-((8aS)-6-chloro-8,8a,9,10,11,12-hexahydropyrazino[2′,1′:3,4][1,4]oxazepino[5,6,7-de]quinazolin-5-yl)-3-fluorophenol

To a solution of tert-butyl (8aS)-6-chloro-5-(2-fluoro-6-hydroxyphenyl)-8a,9,11,12-tetrahydropyrazino[2′,1′:3,4][1,4]oxazepino[5,6,7-de]quinazoline-10(8H)-carboxylate (2.5 g, 5.1 mmol, 1 equiv) in DCM (15 mL) at 0° C. was added TFA (12 mL, 160 mmol, 32 equiv) dropwise then the reaction was warmed to room temperature. After 30 min the reaction mixture was concentrated under reduced pressure. The crude residue was dissolved in MeCN (3 mL), added dropwise to MTBE (450 mL), stirred for 5 min, and filtered. The filter cake was dried under reduced pressure to afford 2-((8aS)-6-chloro-8,8a,9,10,11,12-hexahydropyrazino[2′,1′:3,4][1,4]oxazepino[5,6,7-de]quinazolin-5-yl)-3-fluorophenol (2.28 g, 85% yield, TFA salt) as yellow solid. LCMS (ESI) m/z: [M+H] calcd for C₁₉H₁₇ClFN₄O₂: 387.10; found 387.1. ¹H NMR (400 MHz, Methanol-d₄) δ 8.78 (s, 1H), 7.50 (d, J=2.45 Hz, 1H), 7.27-7.35 (m, 1H), 6.67-6.82 (m, 2H), 5.65 (br d, J=14.67 Hz, 1H), 4.70-4.82 (m, 2H), 4.53 (br d, J=11.25 Hz, 1H), 3.78 (br d, J=12.23 Hz, 1H), 3.56-3.70 (m, 3H), 3.33-3.39 (m, 1H).

### Step 8: Synthesis of ((8aS)-6-chloro-5-(2-fluoro-6-hydroxyphenyl)-8a,9,11,12-tetrahydropyrazino[2′,1′:3,4][1,4]oxazepino[5,6,7-de]quinazolin-10(8H)-yl)((R)-1-tritylaziridin-2-yl)methanone

To a suspension of (R)-1-tritylaziridine-2-carboxylic acid lithium salt (53.6 mg, 160 μmol, 1.2 equiv), 2-((8aS)-6-chloro-8,8a,9,10,11,12-hexahydropyrazino[2′,1′:3,4][1,4]oxazepino[5,6,7-de]quinazolin-5-yl)-3-fluorophenol (52 mg, 134 μmol, 1 equiv), and HATU (60.8 mg, 160 μmol, 1.2 equiv) in DMF (0.7 mL) at 0° C. was added N,N-diisopropylethylamine (47 μL, 268 μmol, 2 equiv). The resulting mixture was stirred for 1 h then diluted with EtOAc (10 mL). The organic phase was washed with 5% aq. citric acid (20 mL), sat. aq. NaHCO₃ (20 mL), sat. aq. NaCl (10 mL), dried over Na₂SO₄, filtered, and concentrated under reduced pressure. The crude product was taken on without further purification. LCMS (ESI) m/z: [M+H] calcd for C₄₁H₃₄ClFN₅O₃: 698.23; found 698.7.

### Step 9: Synthesis of ((R)-aziridin-2-yl((8aS)-6-chloro-5-(2-fluoro-6-hydroxyphenyl)-8a,9,11,12-tetrahydropyrazino[2′,1′:3,4][1,4]oxazepino[5,6,7-de]quinazolin-10(8H)-yl)methanone

To a solution of ((8aS)-6-chloro-5-(2-fluoro-6-hydroxyphenyl)-8a,9,11,12-tetrahydropyrazino[2′,1′:3,4][1,4]oxazepino[5,6,7-de]quinazolin-10(8H)-yl)((R)-1-tritylaziridin-2-yl)methanone (95 mg, 136 μmol, 1 equiv) in DCM (0.7 mL) at 0° C. was added TFA (210 μL, 2.7 mmol, 20 equiv). The resulting mixture was stirred for 5 min then quenched with MeOH (1 mL) and concentrated under reduced pressure. The residue was dissolved in DMSO (0.8 mL) then NEt₃ (380 μL, 2.7 mmol, 20 equiv) was added. The resulting mixture was purified by reverse phase chromatography (5→50% MeCN/H₂O, 0.4% NH₄OH) then repurified by reverse phase chromatography (5→50% MeCN/H₂O, 0.4% NH₄OH) to afford ((R)-aziridin-2-yl((8aS)-6-chloro-5-(2-fluoro-6-hydroxyphenyl-8a,9,11,12-tetrahydropyrazino[2′,1′:3,4][1,4]oxazepino[5,6,7-de]quinazolin-10(8H)-yl)methanone (13.5 mg, 22% yield over 2 steps) as a white solid. LCMS (ESI) m/z: [M+H] calcd for C₂₂H₂₀ClFN₅O₃: 456.13; found 456.6. ¹H NMR (500 MHz, Methanol-d₄) δ 8.55 (s, 1H), 7.42 (s, 1H), 7.28 (q, J=8.2 Hz, 1H), 6.77 (dd, J=8.3, 3.0 Hz, 1H), 6.71 (t, J=8.7 Hz, 1H), 5.11-4.99 (m, 1H), 4.73-4.65 (m, 2H), 4.65-4.57 (m, 1H), 4.57-4.33 (m, 2H), 4.14 (s, 1H), 3.80-3.69 (m, 1H), 3.62-3.48 (m, 1H), 3.23-3.14 (m, 1H), 3.11-2.81 (m, 2H), 1.98-1.80 (m, 2H).

### Example 121—Synthesis of ((S)-aziridin-2-yl)(8aS)-6-chloro-6-(2-fluoro-6-hydroxyphenyl)-8a,9,11,12-tetrahydropyrazino[2′,1′:3,4][1,4]oxazepino[5,6,7-de]quinazolin-10(8H)-yl)methanone

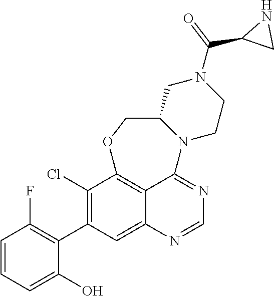 [SMILES: O=C([C@@H]1CN1)N1CCN2c3ncnc4cc(-c5c(O)cccc5F)c(Cl)c(c34)OC[C@@H]2C1]

Synthesized according to the method of example 120, using (S)-1-tritylaziridine-2-carboxylic acid lithium salt in place of (R)-1-tritylaziridine-2-carboxylic acid lithium salt in step 8. LCMS (ESI) m/z: [M+H] calcd for C₂₂H₂₀ClFN₅O₃: 456.13; found 456.5.

### Example 122—Synthesis of ((R)-aziridin-2-yl)((14aR)-11-chloro-10-(5-methyl-1H-indazol-4-yl)-1,3,4,13,14,14a-hexahydro-2H-pyrazino[1′,2′:5,6][1,5]oxazocino[4,3,2-de]quinazolin-2-yl)methanone

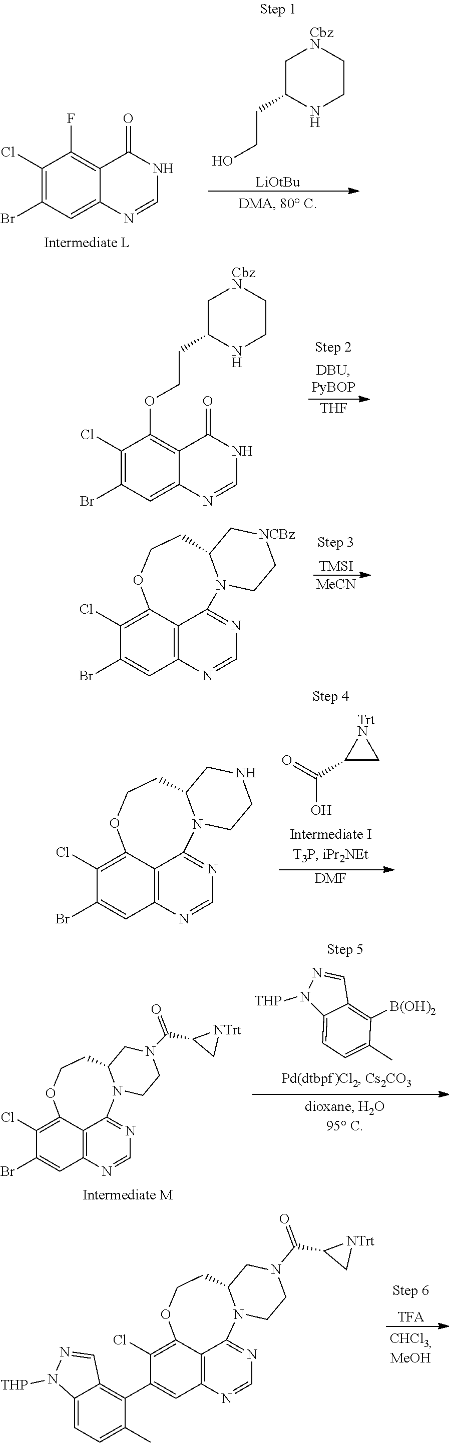

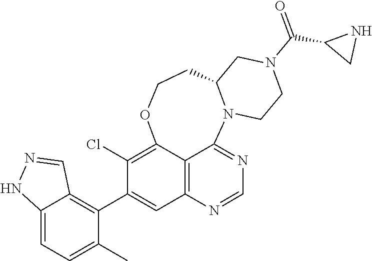 [SMILES: Cc1ccc2[nH]ncc2c1-c1cc2ncnc3c2c(c1Cl)OCC[C@@H]1CN(C(=O)[C@H]2CN2)CCN31]

### Step 1: Synthesis of benzyl (R)-3-(2-((7-bromo-6-chloro-4-oxo-3,4-dihydroquinazolin-5-yl)oxy)ethyl)piperazine-1-carboxylate

To a solution of 7-bromo-6-chloro-5-fluoroquinazolin-4(3H)-one (2.6 g, 9.4 mmol, 1 equiv) and benzyl (R)-3-(2-hydroxyethyl)piperazine-1-carboxylate (3.50 g, 13.2 mmol, 1.4 equiv) in DMA (60 mL) was added lithium tert-butoxide (3.43 g, 42.8 mmol, 4.4 equiv) and the resulting mixture was heated to 80° C. After 2 h the reaction was quenched with MeOH (5 mL) and acidified to pH 2-3 with 1N HCl at 0° C. The resulting solution was diluted with H₂O (80 mL), extracted with DCM (3×50 mL) and the combined organic phase was washed with sat. aq. NaCl (5×80 mL), dried over Na₂SO₄, filtered, and concentrated under reduced pressure to afford benzyl (R)-3-(2-((7-bromo-6-chloro-4-oxo-3,4-dihydroquinazolin-5-yl)oxy)ethyl)piperazine-1-carboxylate as a yellow solid which was used without further purification.

### Step 2: Synthesis of benzyl (R)-10-bromo-11-chloro-1,3,4,13,14,14a-hexahydro-2H-pyrazino[1′,2′:5,6][1,5]oxazocino[4,3,2-de]quinazoline-2-carboxylate

To a solution of benzyl (R)-3-(2-((7-bromo-8-chloro-4-oxo-3,4-dihydroquinazolin-5-yl)oxy)ethyl)piperazine-1-carboxylate (6.0 g, 9.78 mmol, 1 equiv) and PyBop (12.7 g, 24.4 mmol, 2.5 equiv) in THF (140 mL) at 0° C. was added DBU (8.84 mL, 58.6 mmol, 6 equiv) and the resulting mixture was warmed to room temperature. After 2 h the reaction was diluted with EtOAc (800 mL), and washed with 1N HCl (300 mL), H₂O (2×300 mL), sat. aq. NaCl (180 mL), dried over Na₂SO₄, filtered, and concentrated under reduced pressure. The residue was purified by reverse phase chromatography (45%→75% MeCN/H₂O, 10 mM NH₄HCO₃) and fractions containing the desired product were concentrated under reduced pressure to remove MeCN then extracted with EtOAc (5×200 mL). The combined organic phase was concentrated under reduced pressure to afford benzyl (R)-10-bromo-11-chloro-1,3,4,13,14,14a-hexahydro-2H-pyrazino[1′,2′:5,6][1,5]oxazocino[4,3,2-de]quinazoline-2-carboxylate (4.66 g, 98% yield over two steps) as white solid. ¹H NMR (400 MHz, CDCl₃) δ 8.48 (s, 1H), 7.89 (s, 1H), 7.43-7.29 (m, 5H), 5.25-5.08 (m, 2H), 4.86-4.55 (m, 1H), 4.45 (br s, 2H), 4.06-3.45 (m, 6H), 2.31-2.16 (m, 1H), 2.07-1.93 (m, 1H).

### Step 3: Synthesis of (R)-10-bromo-11-chloro-1,3,4,13,14,14a-hexahydro-2H-pyrazino[1′,2′:5,6][1,5]oxazocino[4,3,2-de]quinazoline

To a solution of benzyl (R)-10-bromo-11-chloro-1,3,4,13,14,14a-hexahydro-2H-pyrazino[1′,2′:5,6][1,5]oxazocino[4,3,2-de]quinazoline-2-carboxylate (2.0 g, 4.0 mmol, 1 equiv) in MeCN (40 mL) was added TMSI (1.62 mL, 11.9 mmol, 3 equiv). After 2 h the reaction mixture was quenched with MeOH (20 mL) and acidified to pH to 5 with 1N HCl at 0° C. The resulting solution was diluted with H₂O (50 mL) and washed with MTBE (3×30 mL). The aqueous phase was then basified to pH 8-9 with 1N NaOH at 0° C. and extracted with DCM (3×30 mL). The combined organic phase was washed with sat. aq. NaCl (10 mL), dried over Na₂SO₄, filtered, and concentrated under reduced pressure to (R)-10-bromo-11-chloro-1,3,4,13,14,14a-hexahydro-2H-pyrazino[1′,2′:5,6][1,5]oxazocino[4,3,2-de]quinazoline (1.3 g, 88% yield) as yellow solid which was used without further purification. ¹H NMR (400 MHz, CDCl₃) δ 8.42 (s, 1H), 7.83 (s, 1H), 5.04 (br, 1H), 4.62-4.50 (m, 1H), 4.31 (dt, J=11.2, 3.1 Hz, 1H), 3.85-3.74 (m, 1H), 3.31 (t, J=10.7 Hz, 1H), 3.23-3.11 (m, 1H), 2.97 (d, J=3.4 Hz, 2H), 2.86 (dt, J=11.4, 3.4 Hz, 1H), 2.60 (t, J=12.3 Hz, 1H), 2.02-1.93 (m, 2H).

### Step 4: Synthesis ((R)-10-bromo-11-chloro-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone

To a solution of (R)-10-bromo-11-chloro-1,3,4,13,14,14a-hexahydro-2H-pyrazino[1′,2′:5,6][1,5]oxazocino[4,3,2-de]quinazoline (350 mg, 0.947 mmol, 1 equiv) and (R)-1-tritylaziridine-2-carboxylic acid (1.56 g, 4.73 mmol, 5 equiv) in DMF (3.5 mL) at 0° C. was added N,N-diisopropylethylamine (1.15 mL, 6.63 mmol, 7 equiv) and T₃P (845 □L, 1.42 mmol, 50% solution, 1.5 equiv) and the resulting mixture was warmed to room temperature. After 1 h the reaction mixture was diluted with H₂O (20 mL) and extracted into EtOAc (2×5 mL). The combined organic phase was washed with sat. aq. NaCl (2×10 mL), dried over Na₂SO₄, filtered, and concentrated under reduced pressure. The residue was purified by silica gel chromatography (0→100% EtOAc/petroleum ether) to ((R)-10-bromo-11-chloro-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone (550 mg, 85% yield) as brown solid. LCMS (ESI) m/z: [M+H] calcd for C₃₆H₃₂BrClN₅O₂: 680.13; found 680.1. ¹H NMR (400 MHz, Methanol-d₄) δ 8.37-8.26 (m, 1H), 7.82-7.69 (m, 1H), 7.54-7.51 (m, 2H), 7.46 (td, J=4.9, 2.5 Hz, 4H), 7.34-7.29 (m, 3H), 7.26-7.23 (m, 6H), 4.78-4.69 (m, 0.5H), 4.58-4.50 (m, 0.5H), 4.49-4.43 (m, 1H), 4.37-4.31 (m, 1H), 4.24-4.15 (m, 0.5H), 4.00-3.92 (m, 0.5H), 3.88-3.81 (m, 1.5H), 3.73-3.65 (m, 1.5H), 3.57-3.44 (m, 2H), 2.34-2.14 (m, 2H), 2.05 (td, J=6.2, 3.2 Hz, 1.5H), 1.48-1.38 (m, 1.5H).

### Step 5: Synthesis of ((14aR)-11-chloro-10-(5-methyl-1-(tetrahydro-2H-pyran-2-yl)-1H-indazol-4-yl)-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone

To a solution of ((R)-10-bromo-11-chloro-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone (550 mg, 0.808 mmol, 1 equiv) in dioxane (5.5 mL) and H₂O (280 □L) was added (5-methyl-1-(tetrahydro-2H-pyran-2-yl)-1H-indazol-4-yl)boronic acid (420 mg, 1.62 mmol, 2 equiv), Pd(dtbpf)Cl₂ (52.6 mg, 80.8 □mol, 0.1 equiv) and Cs₂CO₃ (789 mg, 2.42 mmol, 3 equiv) and the resulting mixture was heated to 95° C. After 2 h the reaction mixture was diluted with EtOAc (20 mL), filtered, and added to H₂O (50 mL). The solution was extracted into EtOAc (3×20 mL) and the combined organic phase was washed with sat. aq. NaCl (50 mL), dried over Na₂SO₄, filtered, and concentrated under reduced pressure. The residue was purified by silica gel chromatography (0→100% EtOAc/petroleum ether) to afford ((14aR)-11-chloro-10-(5-methyl-1-(tetrahydro-2H-pyran-2-yl)-1H-indazol-4-yl)-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone (0.4 g, 59% yield) as brown solid. LCMS (ESI) m/z: [M+H] calcd for C₄₉H₄₇ClN₇O₃: 816.34; found 816.3. ¹H NMR (400 MHz, CDCl₃) δ 8.56-8.50 (m, 1H), 7.61-7.53 (m, 9H), 7.39-7.28 (m, 8H), 7.26-7.22 (m, 2H), 5.74 (dt, J=6.8, 2.6 Hz, 1H), 4.80-4.71 (m, 0.5H), 4.54-4.39 (m, 2H), 4.37-4.27 (m, 1H), 4.09-3.99 (m, 1.5H), 3.99-3.92 (m, 0.5H), 3.90-3.85 (m, 1H), 3.83-3.64 (m, 3H), 3.61-3.44 (m, 2H), 2.65-2.52 (m, 1H), 2.46-2.40 (m, 1H), 2.27-2.22 (m, 2.5H), 2.21-2.08 (m, 4H), 2.01-1.92 (m, 1H), 1.83-1.75 (m, 2H), 1.70-1.64 (m, 1H), 1.47-1.41 (m, 1H).

### Step 6: Synthesis of ((R)-aziridin-2-yl)((14aR)-11-chloro-10-(5-methyl-1H-indazol-4-yl)-1,3,4,13,14,14a-hexahydro-2H-pyrazino[1′,2′:5,6][1,5]oxazocino[4,3,2-de]quinazolin-2-yl)methanone

To a solution of ((14aR)-11-chloro-10-(5-methyl-1-(tetrahydro-2H-pyran-2-yl)-1H-indazol-4-yl)-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone (200 mg, 0.245 mmol, 1 equiv) in CHCl₃ (1 mL) and MeOH (1 mL) was added TFA (2.45 mL, 33.1 mmol, 135 equiv) at 0° C. After 30 min the reaction was warmed to room temperature. After 3.5 h the reaction was added sat. aq. NaHCO₃ (100 mL) at ° C. The solution was extracted with EtOAc (2×40 mL) and the combined organic phase was washed with sat. aq. NaCl (40 mL), dried over Na₂SO₄, filtered, and concentrated under reduced pressure. The residue was purified by reserve phase chromatography (20%→40% MeCN/H₂O, 10 mM NH₄HCO₃) to afford ((R)-aziridin-2-yl((14aR)-11-chloro-10-(5-methyl-1H-indazol-4-yl)-1,3,4,13,14,14a-hexahydro-2H-pyrazino[1′,2′:5,6][1,5]oxazocino[4,3,2-de]quinazolin-2-yl)methanone (50.5 mg, 41% yield) as a white solid. LCMS (ESI) m/z: [M+H] calcd for C₂₅H₂₅ClN₇O₂: 490.17; found 490.2; ¹H NMR (400 MHz, Methanol d-₄) δ 8.49-8.42 (m, 1H), 7.57-7.47 (m, 2H), 7.44-7.33 (m, 2H), 4.98-4.88 (m, 1H), 4.63-4.49 (m, 2H), 4.21-4.04 (m, 2.5H), 4.03-3.94 (m, 1H), 3.92-3.75 (m, 1.5H), 3.75-3.59 (m, 1H), 2.99-2.91 (m, 1H), 2.49-2.38 (m, 0.5H), 2.37-2.26 (m, 0.5H), 2.25-2.19 (m, 3H), 2.17 (br d, J=7.9 Hz, 0.5H), 2.06-1.97 (m, 0.5H), 1.95-1.87 (m, 1H), 1.86-1.79 (m, 1H).

### Example 123—Synthesis of ((S)-aziridin-2-yl)(14aR)-11-chloro-10-(5-methyl-1H-Indazol-4-yl)-1,3,4,13,14,14a-hexahydro-2H-pyrazino[1′,2′:5,6][1,5]oxazocino[4,3,2-de]quinazolin-2-yl)methanone

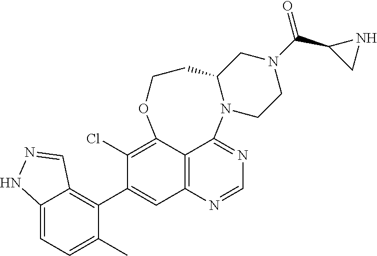 [SMILES: Cc1ccc2[nH]ncc2c1-c1cc2ncnc3c2c(c1Cl)OCC[C@@H]1CN(C(=O)[C@@H]2CN2)CCN31]

Synthesized according to the method of example 122, using (S)-1-tritylaziridine-2-carboxylic acid in place of (R)-1-tritylaziridine-2-carboxylic acid in step 4. LCMS (ESI) m/z: [M+H] calcd for C₂₅H₂₅ClN₇O₂: 490.17; found 490.2.

### Example 124—Synthesis of ((R)-aziridin-2-yl)(14aR)-11-chloro-10-(2-fluoro-6-hydroxyphenyl)-1,3,4,13,14,14a-hexahydro-2H-pyrazino[1′,2′:5,6][1,5]oxazocino[4,3,2-de]quinazolin-2-yl)methanone

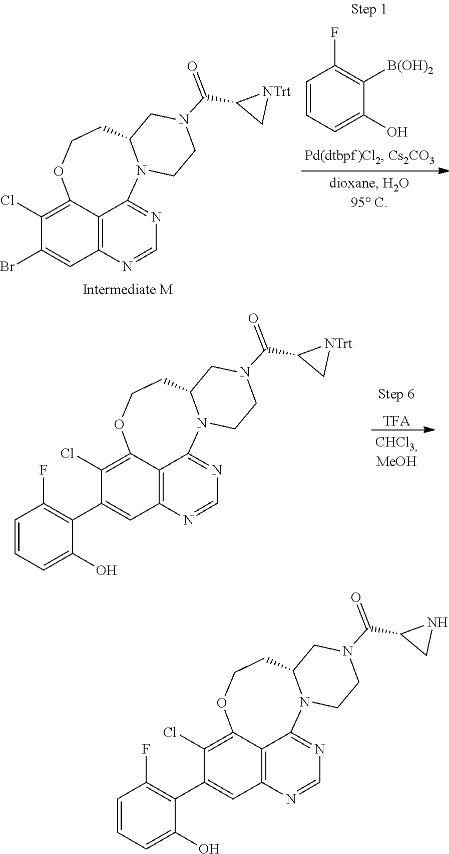

### Step 1: Synthesis of ((14aR)-11-chloro-10-(2-fluoro-6-hydroxyphenyl)-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone

To a solution of ((R)-10-bromo-11-chloro-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone (1 g, 1.47 mmol, 1 equiv) and (2-fluoro-6-hydroxyphenyl)boronic acid (687 mg, 4.41 mmol, 3 equiv) in dioxane (10 mL) and H₂O (0.5 mL) was added Pd(dtbpf)Cl₂ (95.7 mg, 147 □mol, 0.1 equiv) and Cs₂CO₃ (1.44 g, 4.41 mmol, 3 equiv). The resulting mixture was heated at 95° C. for 2 h, then diluted with EtOAc (100 mL), filtered and added to H₂O (300 mL). The separated aqueous phase was extracted with EtOAc (3×100 mL) and the combined organic phase was washed with sat. aq. NaCl, dried over Na₂SO₄, filtered, and concentrated under reduced pressure to afford ((14aR)-11-chloro-10-(2-fluoro-6-hydroxyphenyl)-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone as a brown solid which was used without further purification.

### Step 2: Synthesis of ((R)-aziridin-2-yl)((14aR)-11-chloro-10-(2-fluoro-6-hydroxyphenyl)-1,3,4,13,14,14a-hexahydro-2H-pyrazino[1′,2′:5,6][1,5]oxazocino[4,3,2-de]quinazolin-2-yl)methanone

To a solution of ((14aR)-11-chloro-10-(2-fluoro-6-hydroxyphenyl)-1,3,4,13,14,14a-hexahydro-2H-pyrazino[1′,2′:5,6][1,5]oxazocino[4,3,2-de]quinazolin-2-yl)((R)-1-tritylaziridin-2-yl)methanone (1.31 g, 1.84 mmol, 1 equiv) in CHCl₃ (6.5 mL) and MeOH (6.5 mL) at 0° C. was added TFA (5.41 mL, 70.7 mmol, 38 equiv). After 30 min the reaction was quenched with sat. aq. NaHCO₃ (200 mL) at 0° C. The aqueous phase was extracted with EtOAc (2×70 mL) and the combined organic phase was washed with sat. aq. NaCl (70 mL), dried over Na₂SO₄, filtered, and concentrated under reduced pressure. The residue was purified by reserve phase chromatography (30%→52% MeCN/H₂O, 10 mM NH₄HCO₃) and further purified by additional reserve phase chromatography (10%→40% MeCN/H₂O, 10 mM NH₄HCO₃) to afford ((R)-aziridin-2-yl)((14aR)-11-chloro-10-(2-fluoro-6-hydroxyphenyl-1,3,4,13,14,14a-hexahydro-2H-pyrazino[1′,2′:5,6][1,5]oxazocino[4,3,2-de]quinazolin-2-yl)methanone (33 mg, 4.8% yield over two steps) as a white solid. LCMS (ESI) m/z: [M+H] calcd for C₂₃H₂₂ClFN₅O₃: 470.13; found 470.1. ¹H NMR (400 MHz, Methanol-d₄) δ 8.47-8.38 (m, 1H), 7.46-7.38 (m, 1H), 7.30-7.22 (m, 1H), 6.79-6.64 (m, 2H), 4.86-4.74 (m, 1H), 4.56-4.45 (m, 2H), 4.18-3.97 (m, 3H), 3.96-3.83 (m, 1H), 3.81-3.61 (m, 2H), 2.99-2.88 (m, 1H), 2.47-2.22 (m, 1H), 2.16-2.07 (m, 0.5H), 2.01-1.87 (m, 1.5H), 1.85-1.74 (m, 1H).

### Examples 125 to 180—Synthesis of Exemplary Compounds

The following table of compounds were prepared using the aforementioned methods or variations thereof, as is known to those of skill in the art.

TABLE 3a
Exemplary Compounds Prepared by Methods of the Present Invention

| | | Molecular | Calculated | Observed MW |
|---|---|---|---|---|
| Ex. # | Structure | Formula | MW | LCMS (ESI) m/z |
| 125 | 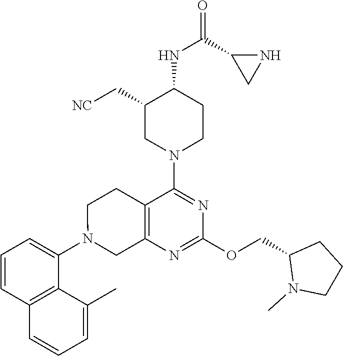 [SMILES: Cc1cccc2cccc(N3CCc4c(nc(OC[C@@H]5CCCN5C)nc4N4CC[C@@H](NC(=O)[C@H]5CN5)[C@@H](CC#N)C4)C3)c12] | C₃₄H₄₂N₈O₂ | [M + H] = 595.35 | [M + H] = 595.3 |
| 126 | 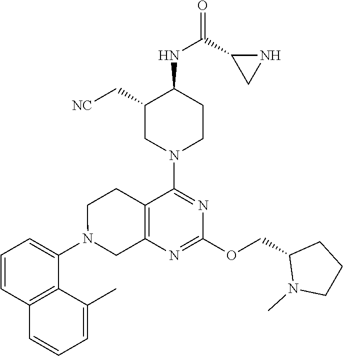 [SMILES: Cc1cccc2cccc(N3CCc4c(nc(OC[C@@H]5CCCN5C)nc4N4CC[C@H](NC(=O)[C@H]5CN5)[C@@H](CC#N)C4)C3)c12] | C₃₄H₄₂N₈O₂ | [M + H] = 595.35 | [M + H] = 595.4 |
| 127 | 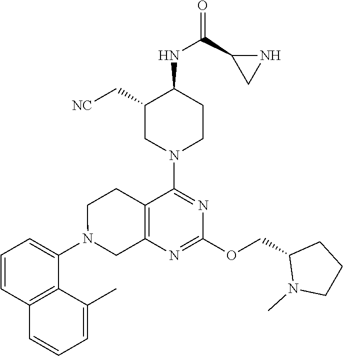 [SMILES: Cc1cccc2cccc(N3CCc4c(nc(OC[C@@H]5CCCN5C)nc4N4CC[C@H](NC(=O)[C@@H]5CN5)[C@@H](CC#N)C4)C3)c12] | C₃₄H₄₂N₈O₂ | [M + H] = 595.35 | [M + H] = 595.4 |
| 128 | 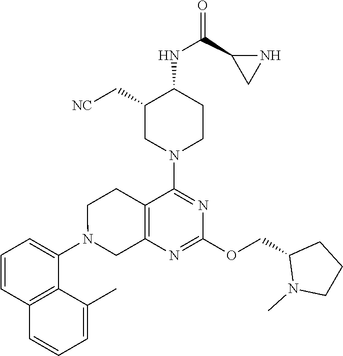 [SMILES: Cc1cccc2cccc(N3CCc4c(nc(OC[C@@H]5CCCN5C)nc4N4CC[C@@H](NC(=O)[C@@H]5CN5)[C@@H](CC#N)C4)C3)c12] | C₃₄H₄₂N₈O₂ | [M + H] = 595.35 | [M + H] = 595.4 |
| 129 | 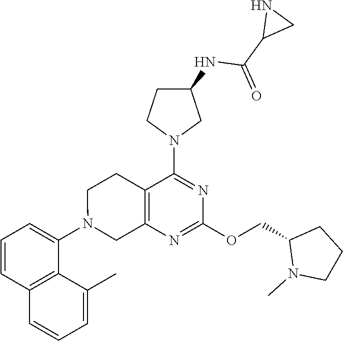 [SMILES: Cc1cccc2cccc(N3CCc4c(nc(OC[C@@H]5CCCN5C)nc4N4CC[C@@H](NC(=O)C5CN5)C4)C3)c12] | C₃₁H₃₉N₇O₂ | [M + H] = 542.33 | [M + H] = 542.3 |
| 130 | 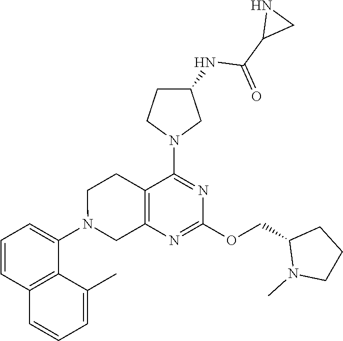 [SMILES: Cc1cccc2cccc(N3CCc4c(nc(OC[C@@H]5CCCN5C)nc4N4CC[C@H](NC(=O)C5CN5)C4)C3)c12] | C₃₁H₃₉N₇O₂ | [M + H] = 542.33 | [M + H] = 542.3 |
| 131 | 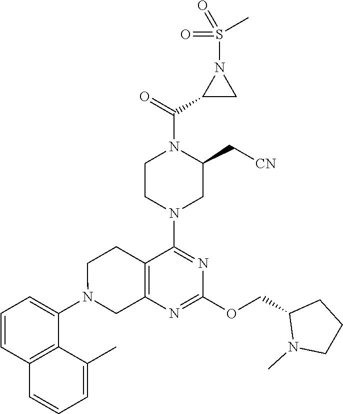 [SMILES: Cc1cccc2cccc(N3CCc4c(nc(OC[C@@H]5CCCN5C)nc4N4CCN(C(=O)[C@H]5CN5S(C)(=O)=O)[C@@H](CC#N)C4)C3)c12] | C₃₄H₄₂N₈O₄S | [M + H] = 659.31 | [M + H] = 659.3 |
| 132 | 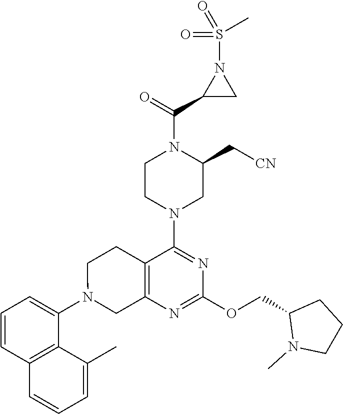 [SMILES: Cc1cccc2cccc(N3CCc4c(nc(OC[C@@H]5CCCN5C)nc4N4CCN(C(=O)[C@@H]5CN5S(C)(=O)=O)[C@@H](CC#N)C4)C3)c12] | C₃₄H₄₂N₈O₄S | [M + H] = 659.31 | [M + H] = 659.4 |
| 133 | 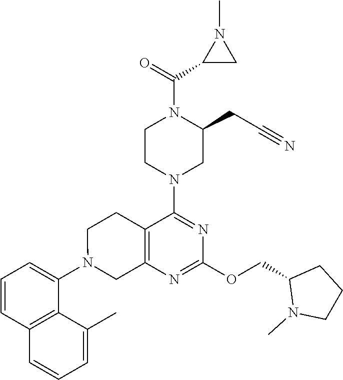 [SMILES: Cc1cccc2cccc(N3CCc4c(nc(OC[C@@H]5CCCN5C)nc4N4CCN(C(=O)[C@H]5CN5C)[C@@H](CC#N)C4)C3)c12] | C₃₄H₄₂N₈O₂ | [M + H] = 595.35 | [M + H] = 595.3 |
| 134 | 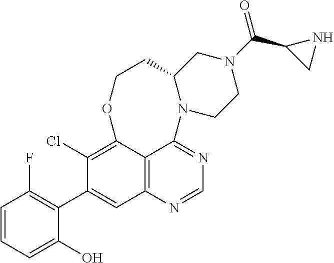 [SMILES: O=C([C@@H]1CN1)N1CCN2c3ncnc4cc(-c5c(O)cccc5F)c(Cl)c(c34)OCC[C@@H]2C1] | C₂₃H₂₁ClFN₅O₃ | [M + H] = 470.14 | [M + H] = 470.2 |
| 135 | 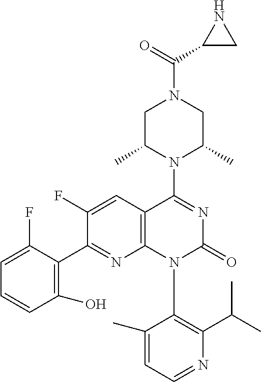 [SMILES: Cc1ccnc(C(C)C)c1-n1c(=O)nc(N2[C@H](C)CN(C(=O)[C@H]3CN3)C[C@@H]2C)c2cc(F)c(-c3c(O)cccc3F)nc21] | C₃₁H₃₃F₂N₇O₃ | [M + H] = 590.27 | [M + H] = 590.3 |
| 136 | 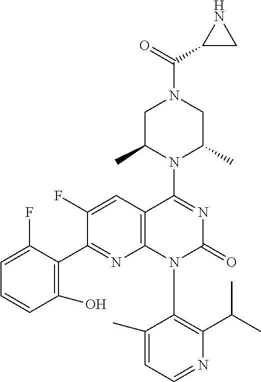 [SMILES: Cc1ccnc(C(C)C)c1-n1c(=O)nc(N2[C@@H](C)CN(C(=O)[C@H]3CN3)C[C@@H]2C)c2cc(F)c(-c3c(O)cccc3F)nc21] | C₃₁H₃₃F₂N₇O₃ | [M + H] = 590.27 | [M + H] = 590.3 |
| 137 | 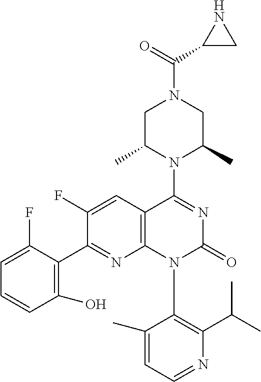 [SMILES: Cc1ccnc(C(C)C)c1-n1c(=O)nc(N2[C@H](C)CN(C(=O)[C@H]3CN3)C[C@H]2C)c2cc(F)c(-c3c(O)cccc3F)nc21] | C₃₁H₃₃F₂N₇O₃ | [M + H] = 590.27 | [M + H] = 590.3 |
| 138 | 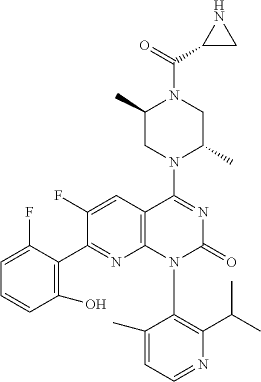 [SMILES: Cc1ccnc(C(C)C)c1-n1c(=O)nc(N2C[C@@H](C)N(C(=O)[C@H]3CN3)C[C@@H]2C)c2cc(F)c(-c3c(O)cccc3F)nc21] | C₃₁H₃₃F₂N₇O₃ | [M + H] = 590.27 | [M + H] = 590.3 |
| 139 | 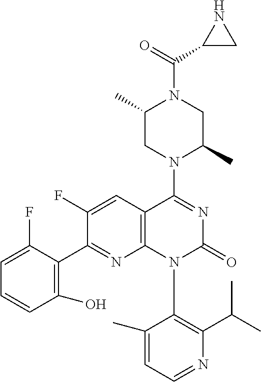 [SMILES: Cc1ccnc(C(C)C)c1-n1c(=O)nc(N2C[C@H](C)N(C(=O)[C@H]3CN3)C[C@H]2C)c2cc(F)c(-c3c(O)cccc3F)nc21] | C₃₁H₃₃F₂N₇O₃ | [M + H] = 590.27 | [M + H] = 590.3 |
| 140 | 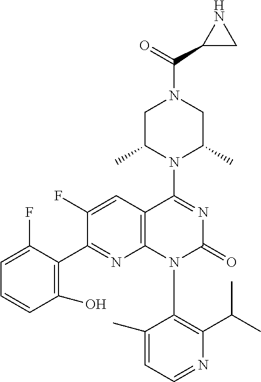 [SMILES: Cc1ccnc(C(C)C)c1-n1c(=O)nc(N2[C@H](C)CN(C(=O)[C@@H]3CN3)C[C@@H]2C)c2cc(F)c(-c3c(O)cccc3F)nc21] | C₃₁H₃₃F₂N₇O₃ | [M + H] = 590.27 | [M + H] = 590.3 |
| 141 | 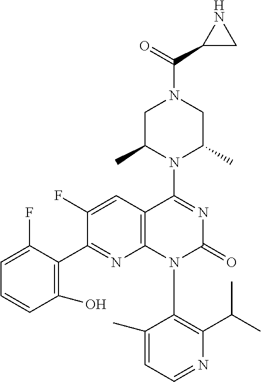 [SMILES: Cc1ccnc(C(C)C)c1-n1c(=O)nc(N2[C@@H](C)CN(C(=O)[C@@H]3CN3)C[C@@H]2C)c2cc(F)c(-c3c(O)cccc3F)nc21] | C₃₁H₃₃F₂N₇O₃ | [M + H] = 590.27 | [M + H] = 590.3 |
| 142 | 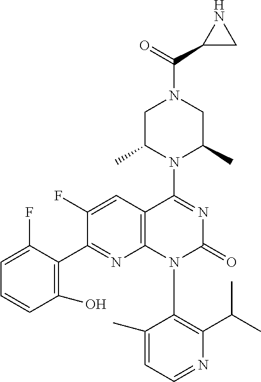 [SMILES: Cc1ccnc(C(C)C)c1-n1c(=O)nc(N2[C@H](C)CN(C(=O)[C@@H]3CN3)C[C@H]2C)c2cc(F)c(-c3c(O)cccc3F)nc21] | C₃₁H₃₃F₂N₇O₃ | [M + H] = 590.27 | [M + H] = 590.4 |
| 143 | 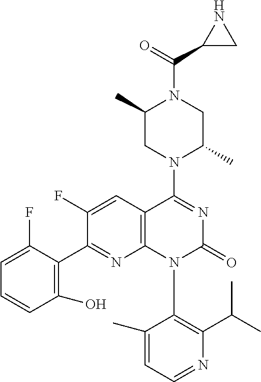 [SMILES: Cc1ccnc(C(C)C)c1-n1c(=O)nc(N2C[C@@H](C)N(C(=O)[C@@H]3CN3)C[C@@H]2C)c2cc(F)c(-c3c(O)cccc3F)nc21] | C₃₁H₃₃F₂N₇O₃ | [M + H] = 590.27 | [M + H] = 590.3 |
| 144 | 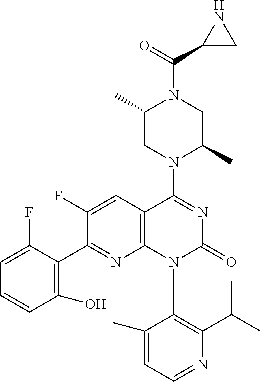 [SMILES: Cc1ccnc(C(C)C)c1-n1c(=O)nc(N2C[C@H](C)N(C(=O)[C@@H]3CN3)C[C@H]2C)c2cc(F)c(-c3c(O)cccc3F)nc21] | C₃₁H₃₃F₂N₇O₃ | [M + H] = 590.27 | [M + H] = 590.3 |
| 145 | 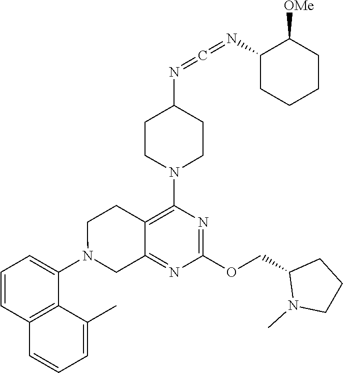 [SMILES: CO[C@H]1CCCC[C@@H]1N=C=NC1CCN(c2nc(OC[C@@H]3CCCN3C)nc3c2CCN(c2cccc4cccc(C)c24)C3)CC1] | C₃₇H₄₉N₇O₂ | [M + H] = 624.40 | [M + H] = 624.4 |
| 146 | 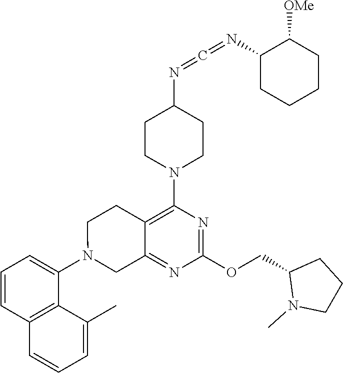 [SMILES: CO[C@@H]1CCCC[C@@H]1N=C=NC1CCN(c2nc(OC[C@@H]3CCCN3C)nc3c2CCN(c2cccc4cccc(C)c24)C3)CC1] | C₃₇H₄₉N₇O₂ | [M + H] = 624.40 | [M + H] = 624.4 |
| 147 | 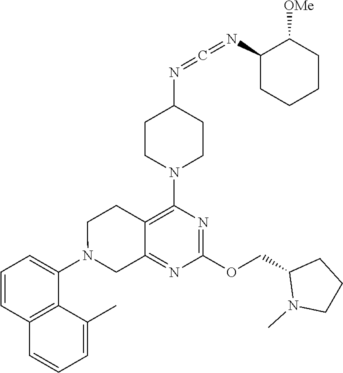 [SMILES: CO[C@@H]1CCCC[C@H]1N=C=NC1CCN(c2nc(OC[C@@H]3CCCN3C)nc3c2CCN(c2cccc4cccc(C)c24)C3)CC1] | C₃₇H₄₉N₇O₂ | [M + H] = 624.40 | [M + H] = 624.4 |
| 148 | 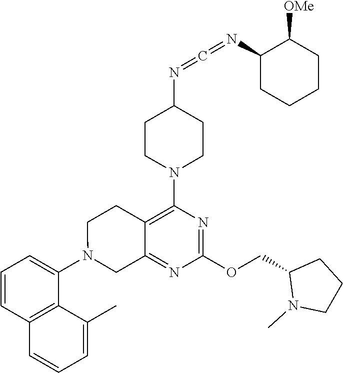 [SMILES: CO[C@H]1CCCC[C@H]1N=C=NC1CCN(c2nc(OC[C@@H]3CCCN3C)nc3c2CCN(c2cccc4cccc(C)c24)C3)CC1] | C₃₇H₄₉N₇O₂ | [M + H] = 624.40 | [M + H] = 624.4 |
| 149* | 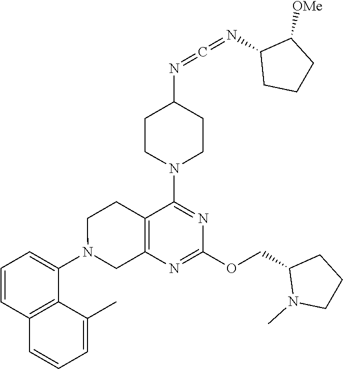 [SMILES: CO[C@@H]1CCC[C@@H]1N=C=NC1CCN(c2nc(OC[C@@H]3CCCN3C)nc3c2CCN(c2cccc4cccc(C)c24)C3)CC1] | C₃₆H₄₇N₇O₂ | [M + H] = 610.39 | [M + H] = 610.4 |
| 150* | 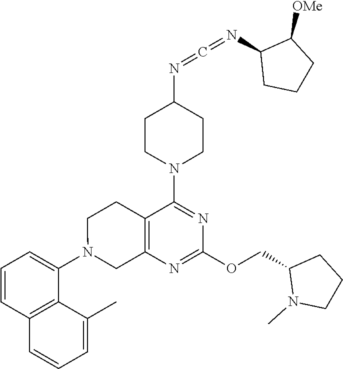 [SMILES: CO[C@H]1CCC[C@H]1N=C=NC1CCN(c2nc(OC[C@@H]3CCCN3C)nc3c2CCN(c2cccc4cccc(C)c24)C3)CC1] | C₃₆H₄₇N₇O₂ | [M + H] = 610.39 | [M + H] = 610.4 |
| 151 | 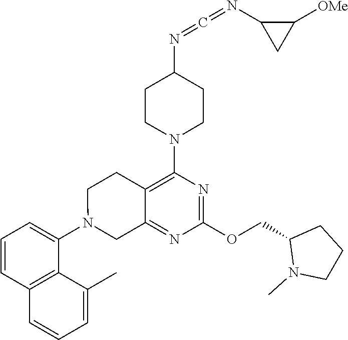 [SMILES: COC1CC1N=C=NC1CCN(c2nc(OC[C@@H]3CCCN3C)nc3c2CCN(c2cccc4cccc(C)c24)C3)CC1] | C₃₄H₄₃N₇O₂ | [M + H] = 582.36 | [M + H] = 582.4 |
| 152 | 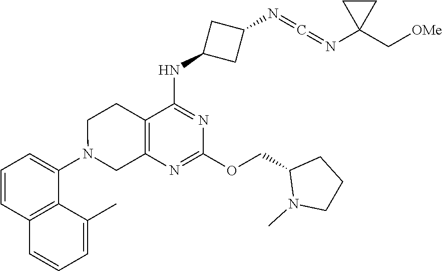 [SMILES: COCC1(N=C=N[C@H]2C[C@H](Nc3nc(OC[C@@H]4CCCN4C)nc4c3CCN(c3cccc5cccc(C)c35)C4)C2)CC1] | C₃₄H₄₃N₇O₂ | [M + H] = 582.36 | [M + H] = 582.4 |
| 153 | 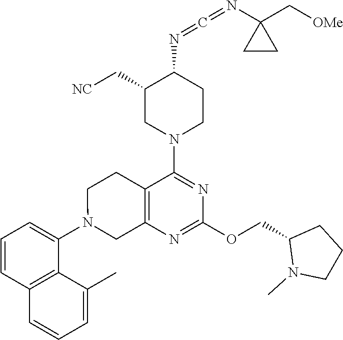 [SMILES: COCC1(N=C=N[C@@H]2CCN(c3nc(OC[C@@H]4CCCN4C)nc4c3CCN(c3cccc5cccc(C)c35)C4)C[C@@H]2CC#N)CC1] | C₃₇H₄₆N₈O₂ | [M + H] = 635.38 | [M + H] = 635.4 |
| 154 | 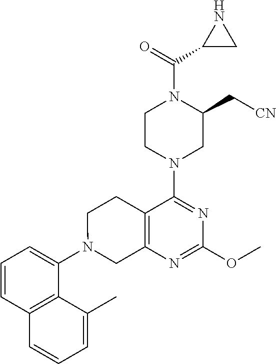 [SMILES: COc1nc2c(c(N3CCN(C(=O)[C@H]4CN4)[C@@H](CC#N)C3)n1)CCN(c1cccc3cccc(C)c13)C2] | C₂₈H₃₁N₇O₂ | [M + H] = 498.26 | [M + H] = 498.3 |
| 155 | 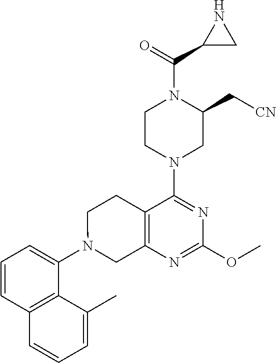 [SMILES: COc1nc2c(c(N3CCN(C(=O)[C@@H]4CN4)[C@@H](CC#N)C3)n1)CCN(c1cccc3cccc(C)c13)C2] | C₂₈H₃₁N₇O₂ | [M + H] = 498.26 | [M + H] = 498.3 |
| 156 | 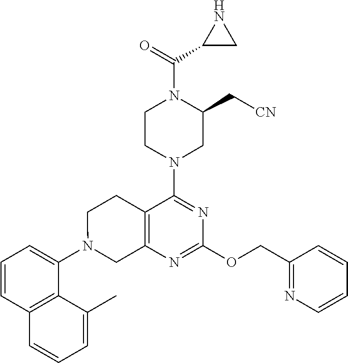 [SMILES: Cc1cccc2cccc(N3CCc4c(nc(OCc5ccccn5)nc4N4CCN(C(=O)[C@H]5CN5)[C@@H](CC#N)C4)C3)c12] | C₃₃H₃₄N₈O₂ | [M + H] = 575.29 | [M + H] = 575.3 |
| 157 | 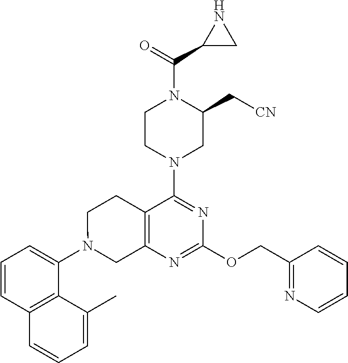 [SMILES: Cc1cccc2cccc(N3CCc4c(nc(OCc5ccccn5)nc4N4CCN(C(=O)[C@@H]5CN5)[C@@H](CC#N)C4)C3)c12] | C₃₃H₃₄N₈O₂ | [M + H] = 575.29 | [M + H] = 575.3 |
| 158 | 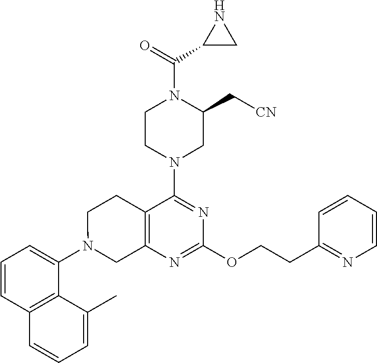 [SMILES: Cc1cccc2cccc(N3CCc4c(nc(OCCc5ccccn5)nc4N4CCN(C(=O)[C@H]5CN5)[C@@H](CC#N)C4)C3)c12] | C₃₄H₃₆N₈O₂ | [M + H] = 589.31 | [M + H] = 589.3 |
| 159 | 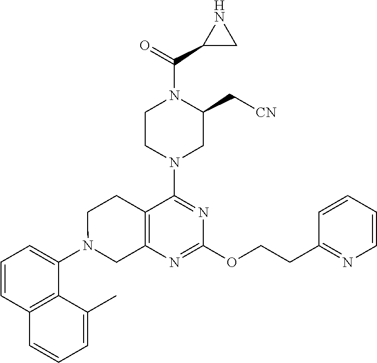 [SMILES: Cc1cccc2cccc(N3CCc4c(nc(OCCc5ccccn5)nc4N4CCN(C(=O)[C@@H]5CN5)[C@@H](CC#N)C4)C3)c12] | C₃₄H₃₆N₈O₂ | [M + H] = 589.31 | [M + H] = 589.3 |
| 160 | 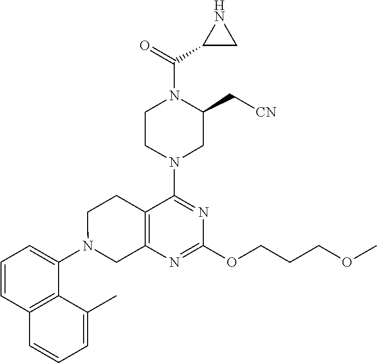 [SMILES: COCCCOc1nc2c(c(N3CCN(C(=O)[C@H]4CN4)[C@@H](CC#N)C3)n1)CCN(c1cccc3cccc(C)c13)C2] | C₃₁H₃₇N₇O₃ | [M + H] = 556.31 | [M + H] = 556.3 |
| 161 | 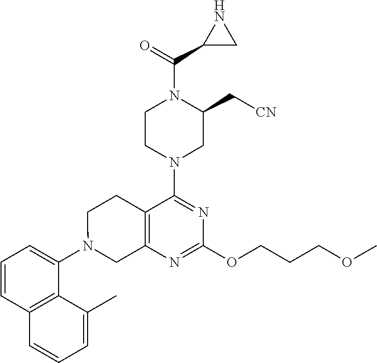 [SMILES: COCCCOc1nc2c(c(N3CCN(C(=O)[C@@H]4CN4)[C@@H](CC#N)C3)n1)CCN(c1cccc3cccc(C)c13)C2] | C₃₁H₃₇N₇O₃ | [M + H] = 556.31 | [M + H] = 556.2 |
| 162 | 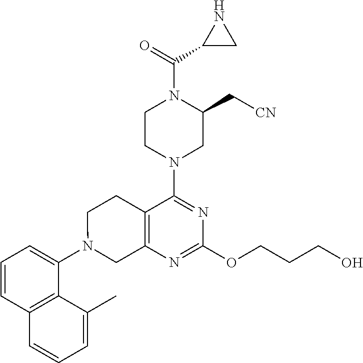 [SMILES: Cc1cccc2cccc(N3CCc4c(nc(OCCCO)nc4N4CCN(C(=O)[C@H]5CN5)[C@@H](CC#N)C4)C3)c12] | C₃₀H₃₅N₇O₃ | [M + H] = 542.29 | [M + H] = 542.1 |
| 163 | 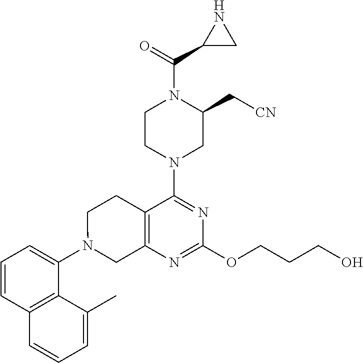 [SMILES: Cc1cccc2cccc(N3CCc4c(nc(OCCCO)nc4N4CCN(C(=O)[C@@H]5CN5)[C@@H](CC#N)C4)C3)c12] | C₃₀H₃₅N₇O₃ | [M + H] = 542.29 | [M + H] = 542.2 |
| 164 | 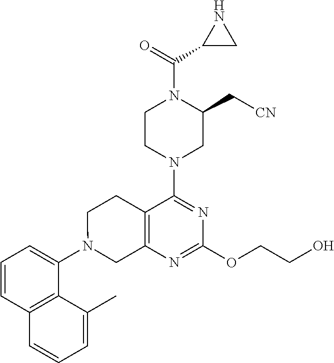 [SMILES: Cc1cccc2cccc(N3CCc4c(nc(OCCO)nc4N4CCN(C(=O)[C@H]5CN5)[C@@H](CC#N)C4)C3)c12] | C₂₉H₃₃N₇O₃ | [M + H] = 528.27 | [M + H] = 528.3 |
| 165 | 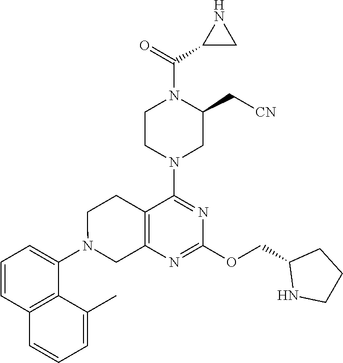 [SMILES: Cc1cccc2cccc(N3CCc4c(nc(OC[C@@H]5CCCN5)nc4N4CCN(C(=O)[C@H]5CN5)[C@@H](CC#N)C4)C3)c12] | C₃₂H₃₈N₈O₂ | [M + H] = 567.32 | [M + H] = 567.2 |
| 166 | 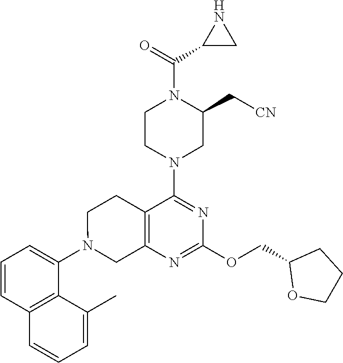 [SMILES: Cc1cccc2cccc(N3CCc4c(nc(OC[C@@H]5CCCO5)nc4N4CCN(C(=O)[C@H]5CN5)[C@@H](CC#N)C4)C3)c12] | C₃₂H₃₇N₇O₃ | [M + H] = 568.61 | [M + H] = 568.5 |
| 167 | 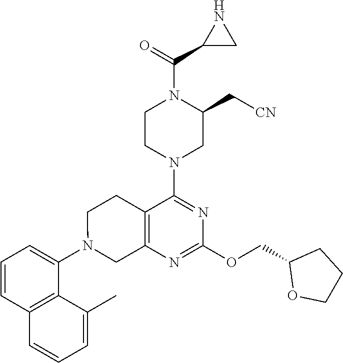 [SMILES: Cc1cccc2cccc(N3CCc4c(nc(OC[C@@H]5CCCO5)nc4N4CCN(C(=O)[C@@H]5CN5)[C@@H](CC#N)C4)C3)c12] | C₃₂H₃₇N₇O₃ | [M + H] = 568.61 | [M + H] = 568.3 |
| 168 | 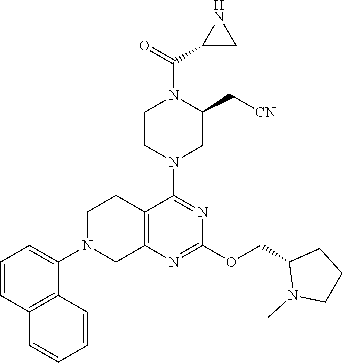 [SMILES: CN1CCC[C@H]1COc1nc2c(c(N3CCN(C(=O)[C@H]4CN4)[C@@H](CC#N)C3)n1)CCN(c1cccc3ccccc13)C2] | C₃₂H₃₈N₈O₂ | [M + H] = 567.62 | [M + H] = 567.3 |
| 169 | 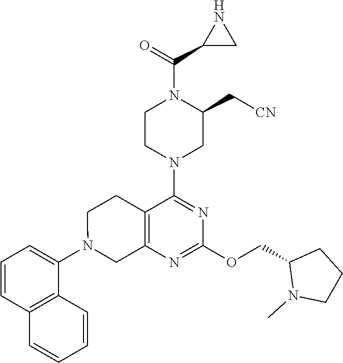 [SMILES: CN1CCC[C@H]1COc1nc2c(c(N3CCN(C(=O)[C@@H]4CN4)[C@@H](CC#N)C3)n1)CCN(c1cccc3ccccc13)C2] | C₃₂H₃₈N₈O₂ | [M + H] = 567.62 | [M + H] = 567.3 |
| 170 | 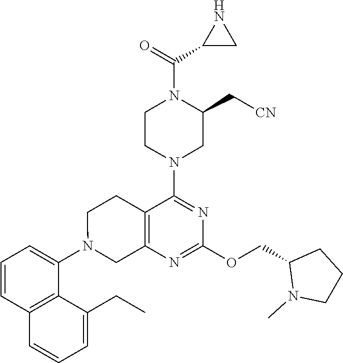 [SMILES: CCc1cccc2cccc(N3CCc4c(nc(OC[C@@H]5CCCN5C)nc4N4CCN(C(=O)[C@H]5CN5)[C@@H](CC#N)C4)C3)c12] | C₃₄H₄₂N₈O₂ | [M + H] = 595.35 | [M + H] = 595.4 |
| 171 | 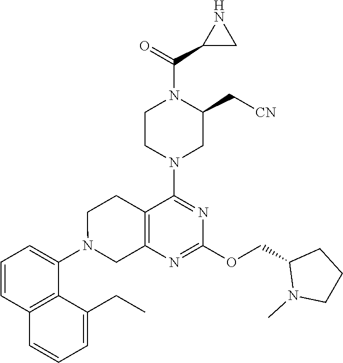 [SMILES: CCc1cccc2cccc(N3CCc4c(nc(OC[C@@H]5CCCN5C)nc4N4CCN(C(=O)[C@@H]5CN5)[C@@H](CC#N)C4)C3)c12] | C₃₄H₄₂N₈O₂ | [M + H] = 595.35 | [M + H] = 595.3 |
| 172 | 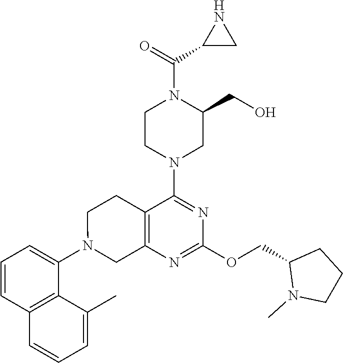 [SMILES: Cc1cccc2cccc(N3CCc4c(nc(OC[C@@H]5CCCN5C)nc4N4CCN(C(=O)[C@H]5CN5)[C@@H](CO)C4)C3)c12] | C₃₂H₄₁N₇O₃ | [M + H] = 572.34 | [M + H] = 572.4 |
| 173 | 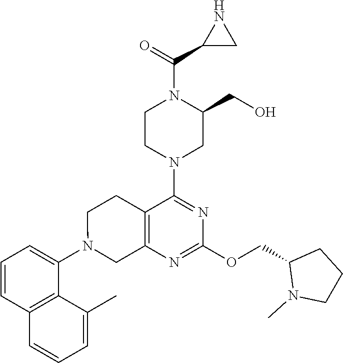 [SMILES: Cc1cccc2cccc(N3CCc4c(nc(OC[C@@H]5CCCN5C)nc4N4CCN(C(=O)[C@@H]5CN5)[C@@H](CO)C4)C3)c12] | C₃₂H₄₁N₇O₃ | [M + H] = 572.34 | [M + H] = 572.4 |
| 174 | 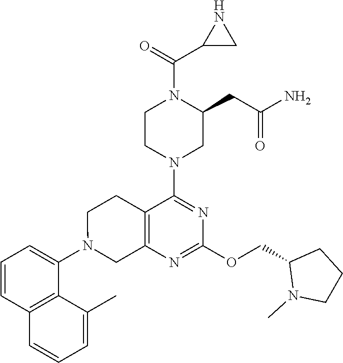 [SMILES: Cc1cccc2cccc(N3CCc4c(nc(OC[C@@H]5CCCN5C)nc4N4CCN(C(=O)C5CN5)[C@@H](CC(N)=O)C4)C3)c12] | C₃₃H₄₃N₈O₃ | [M + H] = 599.35 | [M + H] = 599.4 |
| 175 | 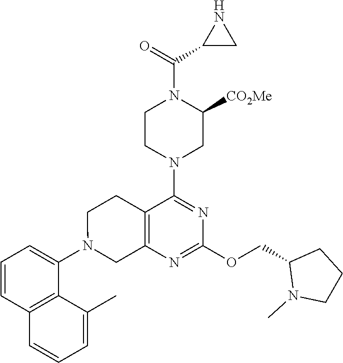 [SMILES: COC(=O)[C@H]1CN(c2nc(OC[C@@H]3CCCN3C)nc3c2CCN(c2cccc4cccc(C)c24)C3)CCN1C(=O)[C@H]1CN1] | C₃₃H₄₁N₇O₄ | [M + H] = 600.33 | [M + H] = 600.4 |
| 176 | 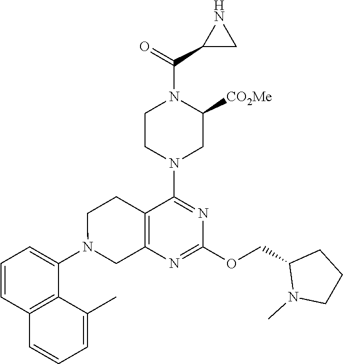 [SMILES: COC(=O)[C@H]1CN(c2nc(OC[C@@H]3CCCN3C)nc3c2CCN(c2cccc4cccc(C)c24)C3)CCN1C(=O)[C@@H]1CN1] | C₃₃H₄₁N₇O₄ | [M + H] = 600.33 | [M + H] = 600.3 |
| 177 | 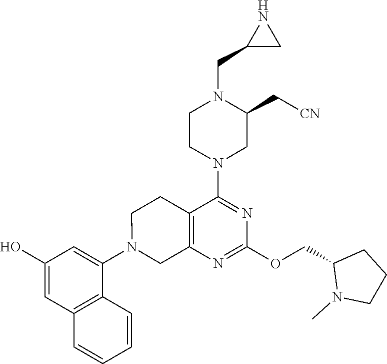 [SMILES: CN1CCC[C@H]1COc1nc2c(c(N3CCN(C[C@@H]4CN4)[C@@H](CC#N)C3)n1)CCN(c1cc(O)cc3ccccc13)C2] | C₃₂H₄₀N₈O₂ | [M + H] = 569.34 | [M + H] = 569.3 |
| 178 | 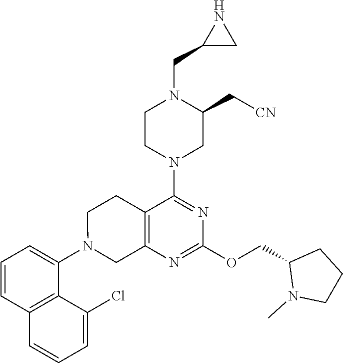 [SMILES: CN1CCC[C@H]1COc1nc2c(c(N3CCN(C[C@@H]4CN4)[C@@H](CC#N)C3)n1)CCN(c1cccc3cccc(Cl)c13)C2] | C₃₂H₃₉ClN₈O | [M + H] = 587.30 | [M + H] = 587.3 |
| 179 | 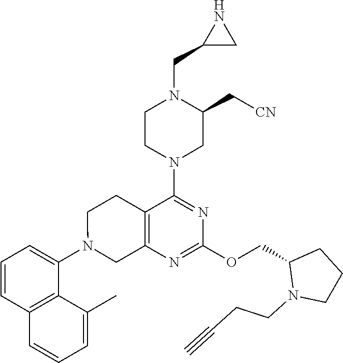 [SMILES: C#CCCN1CCC[C@H]1COc1nc2c(c(N3CCN(C[C@@H]4CN4)[C@@H](CC#N)C3)n1)CCN(c1cccc3cccc(C)c13)C2] | C₃₆H₄₄N₈O | [M + H] = 605.37 | [M + H] = 605.4 |
| 180 | 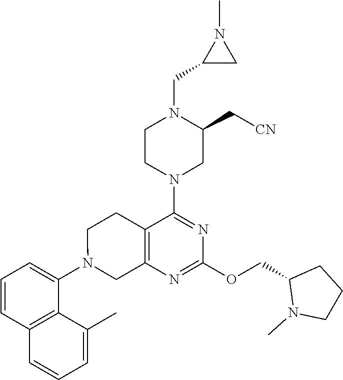 [SMILES: Cc1cccc2cccc(N3CCc4c(nc(OC[C@@H]5CCCN5C)nc4N4CCN(C[C@H]5CN5C)[C@@H](CC#N)C4)C3)c12] | C₃₄H₄₄N₈O | [M + H] = 581.37 | [M + H] = 581.4 |

*Stereochemistry of the cyclopentane is assumed.
Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention.

### Example 181—Synthesis of 2-((S)-1-((R)-1-isopropylaziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

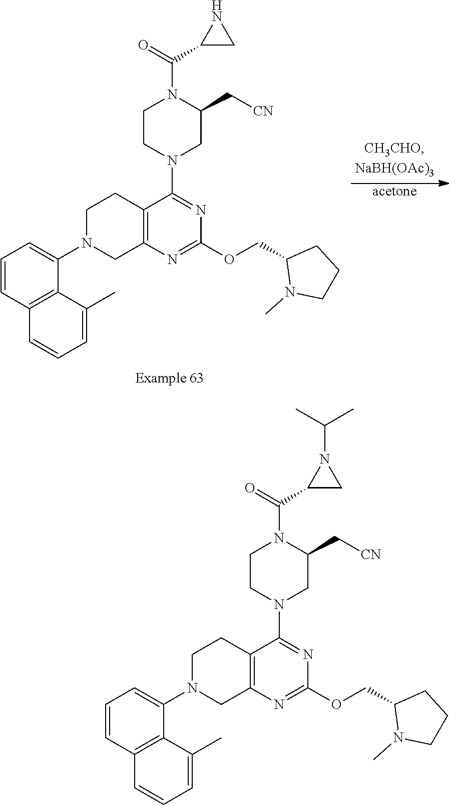 [SMILES: Cc1cccc2cccc(N3CCc4c(nc(OC[C@@H]5CCCN5C)nc4N4CCN(C(=O)[C@H]5CN5)[C@@H](CC#N)C4)C3)c12 ; "acetone" ; Cc1cccc2cccc(N3CCc4c(nc(OC[C@@H]5CCCN5C)nc4N4CCN(C(=O)[C@H]5CN5C(C)C)[C@@H](CC#N)C4)C3)c12]

To a solution of 2-((S)-1-((R)-aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (420 mg, 723.24 μmol, 1 equiv) in acetone (4 mL) was added NaBH(OAc)₃ (919.70 mg, 4.34 mmol, 6 equiv) and AcOH (8.69 mg, 144.65 μmol, 8.27 NL, 0.2 equiv). The resulting mixture was stirred at for 12 h then quenched with H₂O (20 mL). The aqueous layer was exacted with DCM (3×20 mL) and the combined organic phase was washed with sat. aq. NaCl (10 mL), dried over Na₂SO₄, filtered and concentrated under reduced pressure. The crude residue was purified by reverse phase chromatography (40→65% MeCN/H₂O, 10 nM NH₄HCO₃) to afford 2-((S)-1-((R)-1-isopropylaziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (52.22 mg, 11% yield) as white solid. LCMS (ESI) m/z: [M+H] calcd for C₃₆H₄₇N₈O₂: 623.37; found 623.4. ¹H NMR (400 MHz, MeOD) δ 7.76-7.60 (m, 2H), 7.46-7.37 (m, 1H), 7.35-7.21 (m, 3H), 5.10-4.94 (m, 1H), 4.42-3.97 (m, 6H), 3.83-3.47 (m, 3H), 3.29-3.02 (m, 6H), 2.95-2.83 (m, 4H), 2.81-2.61 (m, 3H), 2.49 (d, J=2.2 Hz, 3H), 2.41-2.27 (m, 1H), 2.15-1.95 (m, 2H), 1.85-1.62 (m, 5H), 1.24-1.10 (m, 6H).

### Example 182—Synthesis of methyl (R)-2-((S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl)aziridine-1-carboxylate

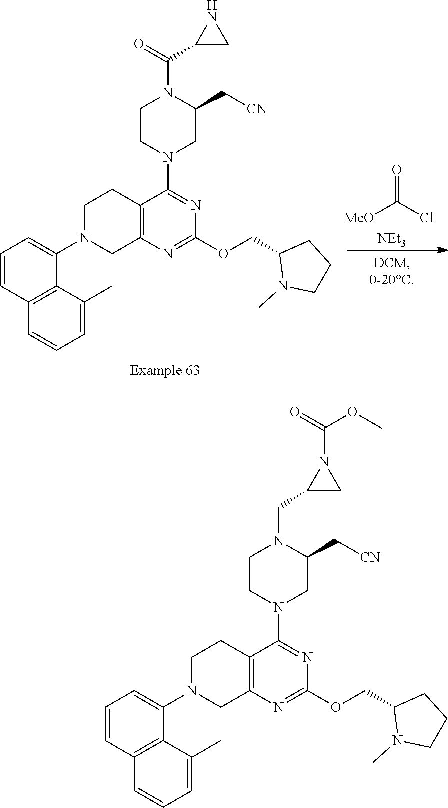 [SMILES: Cc1cccc2cccc(N3CCc4c(nc(OC[C@@H]5CCCN5C)nc4N4CCN(C(=O)[C@H]5CN5)[C@@H](CC#N)C4)C3)c12 ; COC(=O)Cl ; COC(=O)N1C[C@@H]1CN1CCN(c2nc(OC[C@@H]3CCCN3C)nc3c2CCN(c2cccc4cccc(C)c24)C3)C[C@@H]1CC#N ; "NEt3"]

To a solution of 2-((S)-1-((R)-aziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (400 mg, 454 μmol, 1 equiv) and NEt₃ (190 μL, 1.36 mmol, 3 equiv) in DCM (4.0 mL) at 0° C. was added methyl chloroformate (52.8 μL, 681 μmol, 1.5 equiv). The reaction mixture was warmed to room temperature, stirred for 1 h, then poured into ice cold H₂O (10 mL). The aqueous phase was extracted with DCM (3×10 mL) and the combined organic phase was washed with sat. aq. NaCl (10 mL), dried over Na₂SO₄, filtered and concentrated under reduced pressure. The crude residue was purified by reverse phase chromatography (40%→60% MeCN/H₂O, 10 mM NH₄HCO₃) to afford methyl (R)-2-((S)-2-(cyanomethyl)-4-(7-(8-methylnaphthalen-1-yl)-2-(((S)-1-methylpyrrolidin-2-ylmethoxy)-5,6,7,8-tetrahydropyrido[3,4-d]pyrimidin-4-yl)piperazine-1-carbonyl)aziridine-1-carboxylate (51.6 mg, 17% yield) as white solid. LCMS (ESI) m/z: [M+H] calcd for C₃₅H₄₃N₈O₄: 639.34; found 639.4; ¹H NMR (400 MHz, Methanol-d₄) δ=7.74-7.60 (m, 2H), 7.41 (q, J=7.4 Hz, 1H), 7.35-7.22 (m, 3H), 5.10-4.96 (m, 1H), 4.51-4.03 (m, 6H), 3.88-3.45 (m, 8H), 3.26-3.01 (m, 5H), 3.00-2.85 (m, 4H), 2.80-2.65 (m, 2H), 2.61-2.52 (m, 2H), 2.49 (d, J=3.3 Hz, 3H), 2.35 (m, 1H), 2.09 (m, 1H), 1.89-1.63 (m, 3H).

### Example 183—Synthesis of 2-((2S)-1-((1-methylaziridin-2-yl)sulfonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

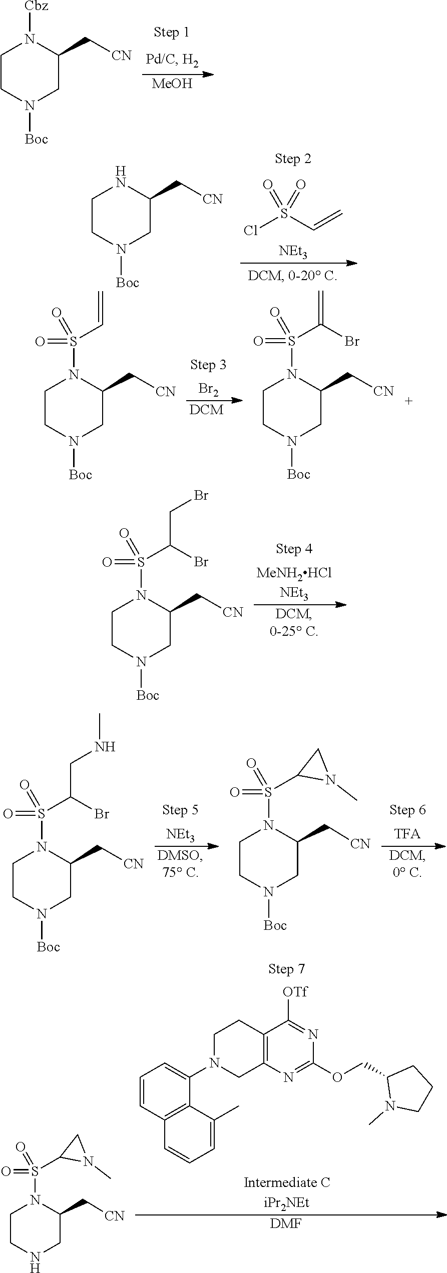

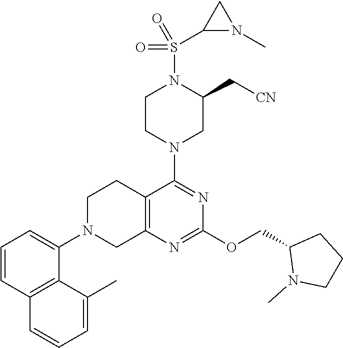 [SMILES: Cc1cccc2cccc(N3CCc4c(nc(OC[C@@H]5CCCN5C)nc4N4CCN(S(=O)(=O)C5CN5C)[C@@H](CC#N)C4)C3)c12]

### Step 1: Synthesis of tert-butyl (S)-3-(cyanomethyl)piperazine-1-carboxylate

To a solution of 1-benzyl 4-(tert-butyl) (S)-2-(cyanomethyl)piperazine-1,4-dicarboxylate (5.0 g, 14 mmol, 1 equiv) in MeOH (50 mL) was added Pd/C (1.5 g, 10 wt. %) and the resulting mixture was stirred under H₂ (15 psi). After 1 h the mixture was filtered through celite, the filter cake was washed with MeOH (300 mL), and the filtrate was concentrated under reduced pressure. The residue was purified by silica gel column chromatography (0→16% MeOH/DCM) to afford tert-butyl (S)-3-(cyanomethyl)piperazine-1-carboxylate (2.7 g, 83% yield) as a gray oil. ¹H NMR (400 MHz, CDCl₃) δ 3.76-4.07 (m, 2H), 2.91-3.06 (m, 3H), 2.57-2.85 (m, 2H), 2.39-2.54 (m, 2H), 1.47 (s, 9H).

### Step 2: Synthesis of tert-butyl (S)-3-(cyanomethyl)-4-(vinylsulfonyl)piperazine-1-carboxylate

To a solution of tert-butyl (S)-3-(cyanomethyl)piperazine-1-carboxylate (2.7 g, 12 mmol, 1 equiv) in DCM (15 mL) at 0° C. was added NEt₃ (13.3 mL, 95.9 mmol, 8 equiv). Ethenesulfonyl chloride (1.82 g, 14.4 mmol, 1.2 equiv) in DCM (12 mL) was added in portions and the resulting mixture was warmed to room temperature. After 1 h the reaction mixture was added to H₂O (50 mL) and extracted with DCM (3×20 mL). The combined organic phase was washed with sat. aq. NaCl (30 mL), dried over Na₂SO₄, filtered, and concentrated under reduced pressure. The residue was purified by silica gel column chromatography (33→100% EtOAc/petroleum ether) to afford (S)-tert-butyl 3-(cyanomethyl)-4-(vinylsulfonyl)piperazine-1-carboxylate (2.38 g, 62% yield) as yellow oil. ¹H NMR (400 MHz, CDCl₃) δ 6.45-6.59 (m, 1H), 6.32 (d, J=16.5 Hz, 1H), 6.04 (d, J=9.70 Hz, 1H), 4.11-4.32 (m, 3H), 3.58-3.62 (m, 1H), 3.02-3.20 (m, 2H), 2.91 (br s, 1H), 2.70-2.72 (m, 2H), 1.49 (s, 9H).

### Step 3: Synthesis of tert-butyl (S)-4-((1-bromovinyl)sulfonyl)-3-(cyanomethyl)piperazine-1-carboxylate and tert-butyl (3S)-3-(cyanomethyl)-4-((1,2-dibromoethyl)sulfonyl)piperazine-1-carboxylate

To a solution of (S)-tert-butyl 3-(cyanomethyl)-4-(vinylsulfonyl)piperazine-1-carboxylate (2.3 g, 7.3 mmol, 1 equiv) in DCM (15 mL) was added Br₂ (714 μL, 13.9 mmol, 1.9 equiv) in DCM (8.0 mL) The resulting mixture was stirred for 3 h then added to H₂O (30 mL) and extracted with DCM (3×20 mL). The combined organic phase was washed with sat. aq. NaCl (20 mL), dried over Na₂SO₄, filtered, and concentrated under reduced pressure. The residue was purified by silica gel column chromatography (25→50% EtOAc/petroleum ether) to afford a mixture of tert-butyl (S)-4-((1-bromovinyl)sulfonyl)-3-(cyanomethyl)piperazine-1-carboxylate and tert-butyl (3S)-3-(cyanomethyl)-4-((1,2-dibromoethyl)sulfonyl)piperazine-1-carboxylate (2.0 g) as a yellow solid.

### Step 4: Synthesis of tert-butyl (3S)-4-((1-bromo-2-(methylamino)ethyl)sulfonyl)-3-(cyanomethyl)piperazine-1-carboxylate

To a solution of a mixture of (S)-tert-butyl 4-((1-bromovinyl)sulfonyl)-3-(cyanomethyl)piperazine-1-carboxylate and (3S)-tert-butyl 3-(cyanomethyl)-4-((1,2-dibromoethyl)sulfonyl)piperazine-1-carboxylate (500 mg) in DCM (5.0 mL) at 0° C. was added NEt₃ (0.77 mL, 5.52 mmol) and methylamine hydrochloride (107 mg, 1.58 mmol). The resulting mixture was warmed to room temperature. After 16 h the mixture was added to H₂O (15 mL) and extracted with DCM (3×10 mL). The combined organic phase was washed with sat. aq. NaCl (10 mL), dried over Na₂SO₄, filtered, and concentrated under reduced pressure to afford tert-butyl (3S)-4-((1-bromo-2-(methylamino)ethyl)sulfonyl)-3-(cyanomethyl)piperazine-1-carboxylate (800 mg, crude) as a yellow solid which was used without further purification. ¹H NMR (400 MHz, CDCl3) δ 4.97-5.12 (m, 1H), 4.07-4.43 (m, 3H), 3.73-3.77 (m, 1H), 3.65 (q, J=7.3 Hz, 1H), 3.34-3.44 (m, 1H), 3.16-3.28 (m, 3H), 2.71-2.86 (m, 2H), 2.48 (d, J=2.6 Hz, 3H), 1.49 (d, J=2.7 Hz, 9H).

### Step 5: Synthesis of tert-butyl (3S)-3-(cyanomethyl)-4-((1-methylaziridin-2-yl)sulfonyl)piperazine-1-carboxylate

To a solution of tert-butyl (3S)-4-((1-bromo-2-(methylamino)ethyl)sulfonyl)-3-(cyanomethyl)piperazine-1-carboxylate (640 mg, 1.50 mmol, 1 equiv) in DMSO (15 mL) was added NEt₃ (3.14 mL, 22.6 mmol, 15 equiv) and the mixture was heated to 75° C. After 16 h the reaction was cooled to room temperature and added to H₂O (50 mL), then extracted with DCM (3×20 mL). The combined organic phase was washed with sat. aq. NaCl (2×15 mL), dried over Na₂SO₄, filtered, and concentrated under reduced pressure. The residue was purified by reverse phase chromatography (10→40% MeCN/H₂O, 0.05% NH₄OH) to afford tert-butyl (3S)-3-(cyanomethyl)-4-((1-methylaziridin-2-yl)sulfonyl)piperazine-1-carboxylate (60 mg, 11% yield) as white solid. ¹H NMR (400 MHz, CDCl₃) δ 4.04-4.45 (m, 3H), 3.78 (br d, J=13.8 Hz, 0.5H), 3.70 (br d, J=13.5 Hz, 0.5H), 3.10-3.23 (m, 2H), 2.62-3.00 (m, 4H), 2.47 (s, 3H) 2.38 (d, J=2.5 Hz, 0.5H), 2.30 (br s, 0.5H), 1.72-1.76 (m, 0.5H) 1.67-1.71 (m, 0.5H), 1.50 (d, J=4.5 Hz, 9H).

### Step 6: Synthesis of 2-((2S)-1-((1-methylaziridin-2-yl)sulfonyl)piperazin-2-yl)acetonitrile

To a solution of tert-butyl (3S)-3-(cyanomethyl)-4-((1-methylaziridin-2-yl)sulfonyl)piperazine-1-carboxylate (30 mg, 87 μmol, 1 equiv) in DCM (0.3 mL) at 0° C. was added TFA (129 μL, 1.74 mmol, 20 equiv). After 2 h the reaction was concentrated under a stream of N₂ to afford 2-((2S)-1-((1-methylaziridin-2-yl)sulfonyl)piperazin-2-yl)acetonitrile (23 mg, crude) as brown oil which was used without further purification.

### Step 7: Synthesis of 2-((2S)-1-((1-methylaziridin-2-yl)sulfonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of 2-((2S)-1-((1-methylaziridin-2-yl)sulfonyl)piperazin-2-yl)acetonitrile (23 mg, 94 μmol, 1 equiv) and (S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate (100 mg, 186 μmol, 2 equiv) in DMF (1 mL) was added N,N-diisopropylethylamine (162 μL, 932 μmol, 5 equiv). The reaction mixture was stirred for 25 min then was added to H₂O (5 mL) and extracted with DCM (3×5 mL). The combined organic phase was washed with sat. aq. NaCl (5 mL), dried over Na₂SO₄, filtered, and concentrated under reduced pressure. The residue was purified by reverse phase chromatography (30→60% MeCN/H₂O, 10 mM NH₄HCO₃) to afford 2-((2S)-1-((1-methylaziridin-2-yl)sulfonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (12 mg, 9.5% yield) as a pale yellow solid. LCMS (ESI) m/z: [M+H] calcd for C₃₃H₄₃N₈O₃S: 631.32; found 631.3. NMR (400 MHz, CDCl₃) δ 7.62-7.72 (m, 2H), 7.31-7.45 (m, 2H), 7.18-7.26 (m, 2H), 4.47-4.54 (m, 0.5H), 4.35-4.40 (m, 1.5H), 4.22-4.30 (m, 1H), 4.09-4.20 (m, 2H), 3.96-4.08 (m, 1H), 3.73-3.93 (m, 2H), 3.44-3.61 (m, 2H), 3.31-3.38 (m, 0.5H), 3.14-3.30 (m, 2.5H), 3.03-3.14 (m, 2H), 2.94-3.03 (m, 2H), 2.92 (s, 3H), 2.82-2.89 (m, 1H), 2.58-2.71 (m, 2H), 2.45-2.52 (m, 5H), 2.39-2.44 (m, 0.5H), 2.33-2.36 (m, 0.5H), 2.23-2.32 (m, 1H), 1.99-2.11 (m, 1H), 1.69-1.88 (m, 4H), 1.23-1.31 (m, 1H).

### Example 184—Synthesis of 2-((S)-1-((S)-2-methylaziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

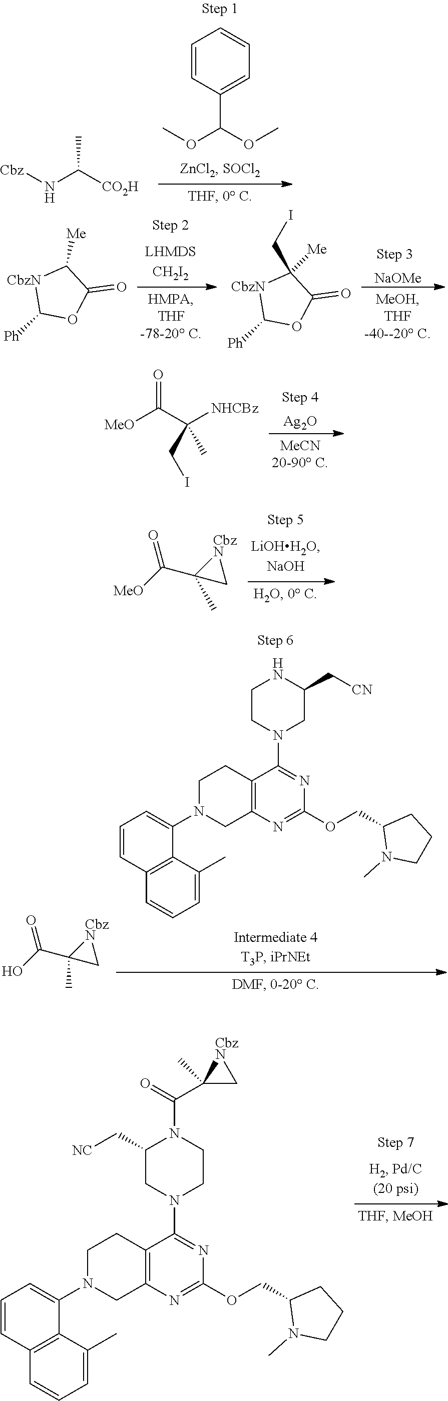

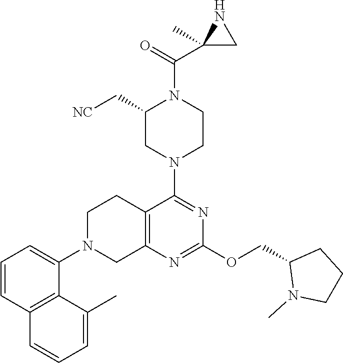 [SMILES: Cc1cccc2cccc(N3CCc4c(nc(OC[C@@H]5CCCN5C)nc4N4CCN(C(=O)[C@]5(C)CN5)[C@@H](CC#N)C4)C3)c12]

### Step 1: Synthesis of benzyl (2R,4R)-4-methyl-5-oxo-2-phenyloxazolidine-3-carboxylate

To a mixture of ((benzyloxy)carbonyl)-D-alanine (5 g, 22.40 mmol, 1 equiv) and (dimethoxymethyl)benzene (3.75 g, 24.64 mmol, 1.1 equiv) in THF (35 mL) at 0° C. was added SOCl₂ (2.93 g, 24.64 mmol, 1.1 equiv). The resulting mixture was stirred for 10 min, then ZnCl₂ (3.36 g, 24.64 mmol, 1.1 equiv) was added. The reaction mixture was stirred at 0° C. for 4 h then concentrated under reduced pressure. The crude residue was purified by silica gel chromatography (0→10% EtOAc/petroleum ether) to afford benzyl (2R,4R)-4-methyl-5-oxo-2-phenyloxazolidine-3-carboxylate (20 g, 64.24 mmol, 57% yield) as yellow oil. ¹H NMR (400 MHz, CDCl₃) δ ppm 7.52-7.14 (m, 10H), 6.66 (s, 1H), 5.26-5.09 (m, 2H), 4.50 (q, J=6.9 Hz, 1H), 1.59 (d, J=7.0 Hz, 3H).

### Step 2: Synthesis of benzyl (2R,4R)-4-(iodomethyl)-4-methyl-5-oxo-2-phenyloxazolidine-3-carboxylate

To a solution of HMPA (13.32 g, 74.34 mmol, 13.06 mL, 4.63 equiv) and LiHMDS (1 M, 16.54 mL, 1.03 equiv) in THF (300 mL) at −78° C. was added dropwise a solution of benzyl (2R,4R)-4-methyl-5-oxo-2-phenyloxazolidine-3-carboxylate (5 g, 16.06 mmol, 1 equiv) in THF (84 mL). After 30 min a solution of CH₂₁I₂ (12.90 g, 48.18 mmol, 3.89 mL, 3 equiv) in THF (33 mL) was added dropwise. The mixture was stirred at −78° C. for 90 min then concentrated under reduced pressure. The crude residue was purified by silica gel chromatography (0→20% EtOAc/petroleum ether) to afford benzyl (2R,4R)-4-(iodomethyl)-4-methyl-5-oxo-2-phenyloxazolidine-3-carboxylate (16 g, 35.46 mmol, 55% yield) as colorless oil. ¹H NMR (400 MHz, CDCl₃) δ ppm 7.47-7.39 (m, 6H), 7.34 (s, 2H), 6.93 (d, J=7.1 Hz, 2H), 6.56 (s, 1H), 5.05 (s, 2H), 4.33 (d, J=10.1 Hz, 1H), 3.64 (d, J=10.1 Hz, 1H), 1.96 (s, 3H).

### Step 3: Synthesis of methyl (R)-2-(((benzyloxy)carbonylamino)-3-iodo-2-methylpropanoate

To a mixture of benzyl (2R,4R)-4-(iodomethyl)-4-methyl-5-oxo-2-phenyloxazolidine-3-carboxylate (16 g, 35.46 mmol, 1 equiv) in THF (90 mL) at −40° C. was added NaOMe (12.77 g, 70.91 mmol, 30% in MeOH, 2 equiv) dropwise over 10 min. After 2 h the resulting mixture was warmed to −20° C. After 1 h the reaction was quenched with H₂O (100 mL) and extracted with EtOAc (3×100 mL). The combined organic phase was washed with sat. aq. NaCl (50 mL), dried over Na₂SO₄, filtered, and concentrated under reduced pressure. The residue was purified by silica gel chromatography (0→20% EtOAc/petroleum ether) to afford methyl (R)-2-(((benzyloxy)carbonyl)amino)-3-iodo-2-methylpropanoate (10 g, 75% yield) as colorless oil. ¹H NMR (400 MHz, CDCl₃) δ ppm 7.40-7.29 (m, 5H), 5.83 (br s, 1H), 5.19-5.06 (m, 2H), 4.16-4.05 (m, 1H), 3.85-3.78 (m, 3H), 3.74 (d, J=10.3 Hz, 1H), 1.73 (s, 3H).

### Step 4: Synthesis of 1-benzyl 2-methyl (S)-2-methylaziridine-1,2-dicarboxylate

To a mixture of methyl (R)-2-(((benzyloxy)carbonyl)amino)-3-iodo-2-methylpropanoate (2 g, 5.30 mmol, 1 equiv) in MeCN (200 mL) was added Ag₂O (3.69 g, 15.91 mmol, 3 equiv). The resulting mixture was heated to 90° C. After 30 min, the reaction mixture was cooled to room temperature, filtered and concentrated under reduced pressure to afford 1-benzyl 2-methyl (S)-2-methylaziridine-1,2-dicarboxylate (5.1 g, 91% yield) as colorless oil which was used without further purification. ¹H NMR (400 MHz, MeOD) δ ppm 7.42-7.29 (m, 5H), 5.15-5.07 (m, 2H), 3.59 (s, 3H), 2.76 (s, 1H), 2.29 (s, 1H), 1.48 (s, 3H).

### Step 5: Synthesis of (S)-1-((benzyloxy)carbonyl)-2-methylaziridine-2-carboxylic Acid

To a solution of 1-benzyl 2-methyl (S)-2-methylaziridine-1,2-dicarboxylate (200 mg, 802.37 μmol, 1 equiv) in MeOH (1 mL) at 0° C. was added a solution of LiOH.H₂O (33.67 mg, 802.37 μmol, 1 equiv) in H₂O (1 mL). After 2 h the reaction mixture was lyophilized to afford (S)-1-((benzyloxy) carbonyl)-2-methylaziridine-2-carboxylic acid (220 mg, crude, Li salt) as white solid which was used without further purification. LCMS (ESI) m/z: [M−H] calcd for C₁₂H₁₂NO₄: 234.08; found 233.9. ¹H NMR (DMSO-d₆, 400 MHz) δ ppm 7.22-7.48 (m, 5H), 5.03 (d, J=12.5 Hz, 1H), 4.88 (d, J=12.5 Hz, 1H), 2.40 (s, 1H), 1.84 (s, 1H), 1.29 (s, 3H).

### Step 6: Synthesis of benzyl (S)-2-((S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl)-2-methylaziridine-1-carboxylate

To a solution of (S)-1-((benzyloxy)carbonyl)-2-methylaziridine-2-carboxylic acid (200 mg, 829.30 μmol, 1 equiv, Li salt) and 2-((S)-4-(7-(8-methylnaphthalen-1-yl)-2-(((S)-1-methyl pyrrolidin-2-yl)methoxy)-5,6,7,8-tetrahydropyrido [3,4-d] pyrimidin-4-yl piperazin-2-yl acetonitrile (254.59 mg, 497.58 μmol, 0.6 equiv) in DMF (2 mL) at 0° C. was added N,N-diisopropylethylamine (535.89 mg, 4.15 mmol, 5 equiv) and T₃P (791.60 mg, 1.24 mmol, 739.81 μL, 50% purity, 1.5 equiv). The resulting mixture was warmed to room temperature. After 2 h, N,N-diisopropylethylamine (535.90 mg, 4.15 mmol, 722.24 μL, 5 equiv) and T₃P (395.80 mg, 1.24 mmol, 50% purity, 1.5 equiv) were added. After 14 h, the reaction mixture was added into cold sat. aq. NH₄Cl (50 mL) and extracted with EtOAc (3×30 mL). The combined organic phase was washed with sat. aq. NaCl (60 mL), dried over anhydrous Na₂SO₄, filtered and concentrated under reduced pressure. The resulting crude product was purified by silica gel chromatography (0→50% MeOH/EtOAc) to afford benzyl (S)-2-((S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl)-2-methylaziridine-1-carboxylate (270 mg, 45% yield) as yellow solid. LCMS (ESI) m/z: [M+H] calcd for C₄₂H₄₉N₈O₄: 729.38; found 729.5.

### Step 7: Synthesis of 2-((S)-1-((S)-2-methylaziridine-2-carbonyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

To a solution of benzyl (S)-2-((S)-2-(cyanomethyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazine-1-carbonyl)-2-methylaziridine-1-carboxylate (200 mg, 274.39 μmol, 1 equiv) in MeOH (2 mL) and THF (2 mL) was added Pd/C (100 mg, 82.32 μmol, 10% purity). The resulting mixture was stirred under H₂ (20 psi). After 1 h, the reaction mixture was filtered and concentrated under reduced pressure. The crude product was purified by reverse phase chromatography (25→55% MeCN/H₂O, 10 nM NH₄HCO₃) to afford 2-((S)-1-((S)-2-methylaziridine-2-carbonyl)-4-(7-(8-methylnaphthalene-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (25 mg, 15% yield) as white solid. LCMS (ESI) m/z: [M+H] calcd for C₃₄H₄₃N₈O₂: 595.34; found 595.4. ¹H NMR (400 MHz, Methanol-d₄) δ 7.69 (d, J=7.95 Hz, 1H), 7.65 (d, J=7.95 Hz, 1H), 7.37-7.44 (m, 1H), 7.26-7.35 (m, 2H), 7.21-7.26 (m, 1H), 5.03 (s, 1H), 4.57 (s, 1H), 4.00-4.42 (m, 6H), 3.63-3.78 (m, 1H), 3.47-3.58 (m, 1H), 3.12-3.27 (m, 4H), 2.99-3.11 (m, 2H), 2.91 (s, 4H), 2.60-2.80 (m, 2H), 2.49 (d, J=2.45 Hz, 3H), 2.34 (qd, J=8.88, 3.67 Hz, 1H), 2.00-2.15 (m, 2H), 1.80 (d, J=7.46 Hz, 3H), 1.64-1.75 (m, 1H), 1.40-1.62 (m, 3H).

### Example 185—Synthesis of 2-((S)-1-(((S)-1-methylaziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

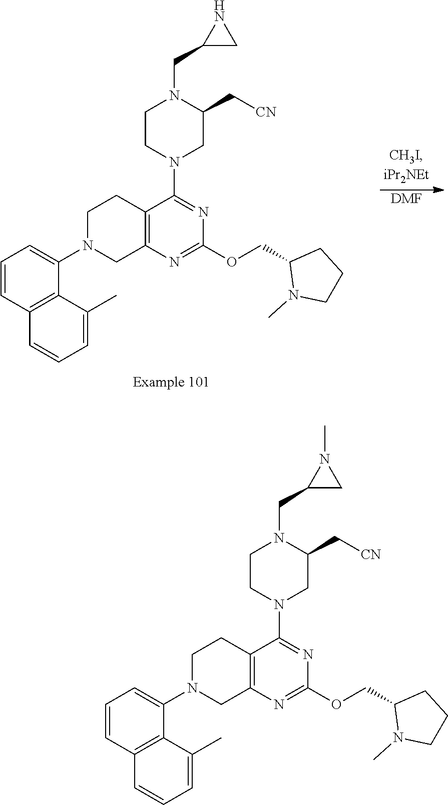

To a solution of 2-((S)-1-(((S)-aziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (650 mg, 840 μmol, 1 equiv) and iodomethane (57 μL, 920 μmol, 1.1 equiv) in DMF (6 mL) was added N,N-diisopropylethylamine (440 μL, 2.5 mmol, 3 eq). After 2 h the reaction was filtered then purified by reverse phase chromatography (30→60% MeCN/H₂O, 0.05% NH₃H₂O, 10Mm NH₄HCO₃) followed by a second purification by reverse phase chromatography (30→60% MeCN/H₂O, 0.05% NH₃H₂O, 10 Mm NH₄HCO₃) to afford 2-((S)-1-(((S)-1-methylaziridin-2-yl)methyl)-4-(7-(8-methylnaphthalen-1-yl)-2-(((S)-1-methylpyrrolidin-2-ylmethoxy)-5,6,7,8-tetrahydropyrido[3,4-d]pyrimidin-4-yl)piperazin-2-yl)acetonitrile (57 mg, 10% yield) as an off-white solid. ¹H NMR (400 MHz, Methanol-d₄) δ 7.68 (br dd, J=7.9, 16.0 Hz, 2H), 7.42 (dt, J=3.9, 7.7 Hz, 1H), 7.36-7.21 (m, 3H), 4.81-4.75 (m, 1H), 4.64 (br dd, J=8.5, 13.6 Hz, 1H), 4.10 (br dd, J=9.5, 17.5 Hz, 2H), 4.03-3.91 (m, 1H), 3.89-3.47 (m, 7H), 3.25-3.15 (m, 3H), 3.11 (s, 3H), 2.91 (s, 4H), 2.87-2.66 (m, 4H), 2.64-2.37 (m, 3H), 2.37-2.11 (m, 3H), 2.11-2.04 (m, 4H), 2.01-1.76 (m, 1H), 1.43-1.22 (m, 1H). LCMS (ESI) m/z: [M+H] calcd for C₃₄H₄₅N₈O: 581.37; found 581.3.

### Example 186—Synthesis of 2-((S)-1-(((R)-1-isopropylaziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile

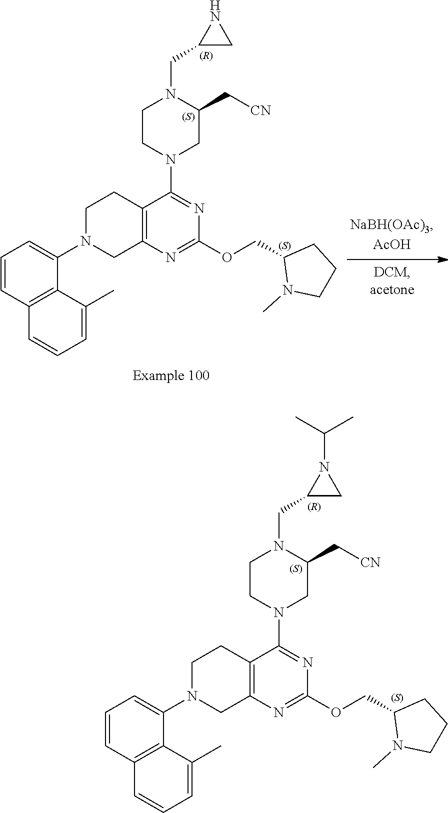

To a solution of 2-((S)-1-(((R)-aziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (200 mg, 352.90 μmol, 1 equiv) in acetone (1 mL) and DCM (1 mL) was added NaBH(OAc)₃ (373.96 mg, 1.76 mmol, 5 equiv) and AcOH (4.24 mg, 70.58 μmol, 0.2 equiv). After 4 h, the reaction was quenched with H₂O (10 mL) and extracted with DCM (3×10 mL). The combined organic phase was washed with sat. aq. NaCl (5 mL), dried over Na₂SO₄, filtered, and concentrated under reduced pressure. The resulting crude material was purified by reverse phase chromatography (55→75% MeCN/H₂O, 10 mM NH₄HCO₃) to afford 2-((S)-1-(((R)-1-isopropylaziridin-2-yl)methyl)-4-(7-(8-methylnaphthalen-1-yl)-2-(((S)-1-methylpyrrolidin-2-yl)methoxy)-5,6,7,8-tetrahydropyrido[3,4-d]pyrimidin-4-yl)piperazin-2-yl)acetonitrile (8.76 mg, 13.96 μmol, 3.9% yield) as a white solid. LCMS (ESI) m/z: [M+H] calcd for C₃₆H₄₉N₈O: 609.40; found 609.5. ¹H NMR (400 MHz, DMSO-d₆) δ 7.69-7.65 (m, 2H), 7.40 (dt, J=4.5, 7.7 Hz, 1H), 7.35-7.21 (m, 3H), 4.42-4.26 (m, 2H), 4.08 (dd, J=10.5, 17.9 Hz, 1H), 3.90 (br dd, J=2.5, 12.9 Hz, 0.5H), 3.83-3.42 (m, 6H), 3.26-3.02 (m, 5H), 2.91 (s, 3H), 2.84-2.60 (m, 5.5H), 2.56-2.44 (m, 4H), 2.41-2.28 (m, 1H), 2.12-2.01 (m, 1H), 1.88-1.54 (m, 6H), 1.49 (d, J=6.6 Hz, 1H), 1.23-1.09 (m, 6H).

### Example 187—Synthesis of 2-((S)-3-((R)-aziridine-2-carbonyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)imidazolidin-4-yl)acetonitrile

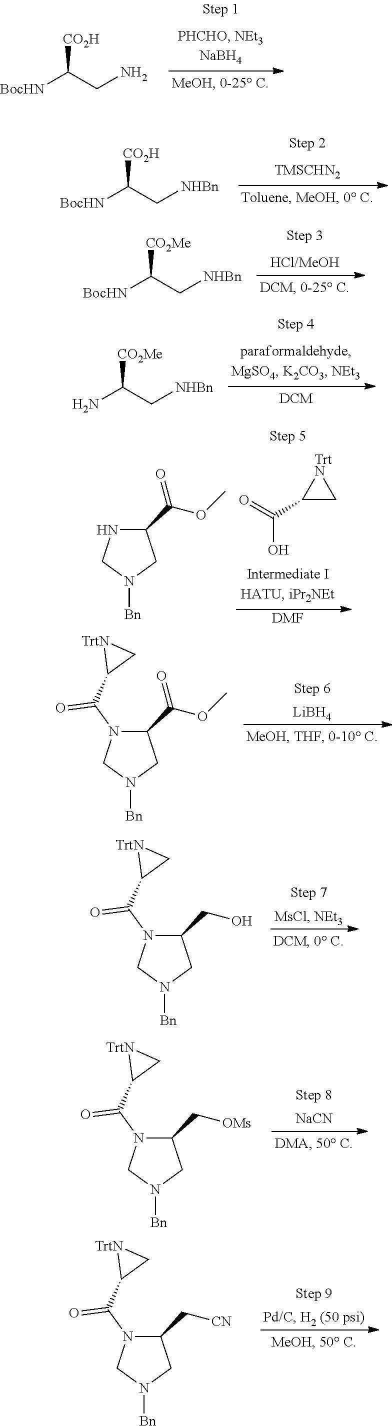

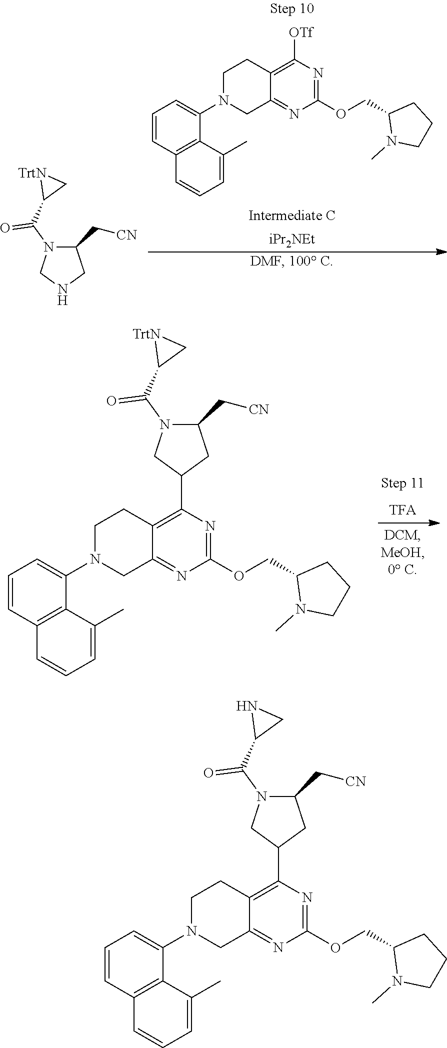

### Step 1: Synthesis of (R)-3-(benzylamino)-2-((tert-butoxycarbonyl)amino)propanoic Acid

To a suspension of (R)-3-amino-2-((tert-butoxycarbonyl)amino)propanoic acid (55 g, 269.31 mmol, 1 equiv) in MeOH (550 mL) at 0° C. was added benzaldehyde (57.16 g, 538.63 mmol, 2 equiv) and NEt₃ (81.75 g, 807.94 mmol, 3 equiv). The resulting suspension was slowly warmed to room temperature. After 1 h, the reaction was cooled to 0° C. and NaBH₄ (30.57 g, 807.94 mmol, 3 equiv) was added in portions. After 1 h, the reaction was diluted with H₂O (80 mL) and concentrated under reduced pressure. The resulting residue was diluted with 0.1M NaOH (1100 mL) and washed with MTBE (2×300 mL). The aqueous phase was acidified with 1M HCl to pH 5-6 and extracted with chloroform (4×600 mL). The combined organic phase was dried over Na₂SO₄, filtered, and concentrated under reduced pressure to afford (R)-3-(benzylamino)-2-((tert-butoxycarbonyl)amino)propanoic acid (66 g, 83% yield) as a white solid which was used without further purification.

### Step 2: Synthesis of methyl (R)-3-(benzylamino)-2-((tert-butoxycarbonyl)amino)propanoate

To a solution of (R)-3-(benzylamino)-2-((tert-butoxycarbonyl)amino)propanoic acid (48 g, 163.07 mmol, 1 equiv) in MeOH (320 mL) and toluene (160 mL) at 0° C. was added TMSCHN₂ (2M, 81.54 mL, 1 equiv). After 1 h, TMSCHN₂ (2 M, 81.54 mL, 1 equiv) was added. After 1 additional hour, TMSCHN₂ (2M, 24.46 mL, 0.3 equiv) was added and the solution was stirred for 1 h. The solution was concentrated under reduced pressure. The resulting crude material was purified by silica gel column chromatography (50→100% EtOAc/petroleum ether) to afford methyl (R)-3-(benzylamino)-2-((tert-butoxycarbonyl)amino)propanoate (15.5 g, 28% yield) as a yellow oil. ¹H NMR (400 MHz, CDCl₃) δ 7.37-7.19 (m, 5H), 5.48-5.36 (m, 1H), 4.45-4.34 (m, 1H), 3.85-3.68 (m, 5H), 3.06-2.90 (m, 2H), 1.44 (s, 9H).

### Step 3: Synthesis of methyl (R)-2-amino-3-(benzylamino)propanoate

To a solution of (R)-3-(benzylamino)-2-((tert-butoxycarbonyl)amino)propanoate (15.5 g, 50.26 mmol, 1 equiv) in DCM (100 mL) at 0° C. was added 4 M HCl in MeOH (251.32 mL, 20 equiv), and the reaction was warmed to room temperature. After 2 h, the mixture was concentrated under reduced pressure to afford methyl (R)-2-amino-3-(benzylamino)propanoate (13 g, 92% yield) as a white solid which was used without further purification. ¹H NMR (400 MHz, Methanol-d₄) δ 7.63 (s, 2H), 7.54-7.39 (m, 3H), 4.27-4.55 (m, 1H), 4.39 (s, 2H), 3.93 (s, 3H), 3.83-3.67 (m, 1H), 3.65-3.48 (m, 1H).

### Step 4: Synthesis of methyl (R)-1-benzylimidazolidine-4-carboxylate

To a solution of (R)-2-amino-3-(benzylamino)propanoate (2.5 g, 8.89 mmol, 1 equiv) in CHCl₃ (25 mL) was added paraformaldehyde (800.20 mg, 26.67 mmol, 3 equiv), MgSO₄ (4.28 g, 35.56 mmol, 4 equiv), K₂CO₃ (3.69 g, 26.67 mmol, 3 equiv), and NEt₃ (4.50 g, 44.46 mmol, 6.19 mL, 5 equiv). After 24 h, the mixture was filtered and concentrated under reduced pressure. The residue was suspended in EtOAc (50 mL), filtered, and concentrated to afford methyl (R)-1-benzylimidazolidine-4-carboxylate (4.4 g, crude) as a yellow oil, which was used without further purification.

### Step 5: Synthesis of methyl (R)-1-benzyl-3-((R)-1-tritylaziridine-2-carbonyl)imidazolidine-4-carboxylate

To a solution of (R)-1-tritylaziridine-2-carboxylic acid (7.04 g, 18.16 mmol, 1 equiv) in DMF (25 mL) was added HATU (6.90 g, 18.16 mmol, 1 equiv), N,N-diisopropylethylamine (4.69 g, 36.32 mmol, 2 equiv) and a solution of methyl (R)-1-benzylimidazolidine-4-carboxylate (4 g, 18.16 mmol, 1 equiv) in DMF (15 mL). After 2 h, the reaction mixture was diluted with H₂O (100 mL) and extracted with EtOAc (3×100 mL). The combined organic phase was washed with sat. aq. NaCl (100 mL), dried over Na₂SO₄, filtered, and concentrated under reduced pressure. The residue was purified by silica gel column chromatography (0→50% EtOAc/petroleum ether) to afford methyl (R)-1-benzyl-3-((R)-1-tritylaziridine-2-carbonyl)imidazolidine-4-carboxylate (4.6 g, 42% yield) as a white solid. ¹H NMR (400 MHz, CDCl₃) δ 7.55-7.16 (m, 20H), 4.05 (d, J=6.2 Hz, 1H), 3.91-3.85 (m, 1H), 3.74 (s, 3H), 3.66-3.56 (m, 2H), 3.43 (d, J=12.3 Hz, 1H), 3.26-3.18 (m, 1H), 3.03-2.93 (m, 1H), 2.41 (s, 1H), 1.65-1.51 (m, 1H), 1.43-1.33 (m, 1H).

### Step 6: Synthesis of ((R)-3-benzyl-5-(hydroxymethyl)imidazolidin-1-yl)((R)-1-tritylaziridin-2-yl)methanone

To a solution of methyl (R)-1-benzyl-3-((R)-1-tritylaziridine-2-carbonyl)imidazolidine-4-carboxylate (3.6 g, 6.77 mmol, 1 equiv) in THF (36 mL) and MeOH (3.69 g, 115.11 mmol, 4.66 mL, 17 equiv) at 0° C. was added LiBH₄ (2 M, 16.50 mL, 4.87 equiv), and the reaction was warmed to 10° C. After 3 h, the mixture was quenched with H₂O (50 mL) and extracted with EtOAc (3×20 mL). The combined organic phase was washed with sat. aq. NaCl (2×9 mL), dried over Na₂SO₄, filtered, and concentrated under reduced pressure. The residue was purified by silica gel column chromatography (10→100% EtOAc/petroleum ether) to afford ((R)-3-benzyl-5-(hydroxymethyl)imidazolidin-1-yl)((R)-1-tritylaziridin-2-yl)methanone (2.33 g, 67% yield) as a white solid. ¹H NMR (400 MHz, CDCl₃) δ 7.50-7.41 (m, 1H), 7.40-7.30 (m, 5H), 7.26-7.05 (m, 13H), 7.03-6.99 (m, 1H), 4.60 (br s, 1H), 4.45-4.32 (m, 1H), 3.81-3.52 (m, 4H), 3.50-3.41 (m, 1H), 3.38-3.30 (m, 1H), 2.99-2.87 (m, 1H), 2.62-2.49 (m, 1H), 2.39-2.33 (m, 1H), 1.49-1.41 (m, 1H), 1.33-1.26 (m, 1H).

### Step 7: Synthesis of ((R)-1-benzyl-3-((R)-1-tritylaziridine-2-carbonyl)imidazolidin-4-yl)methyl methanesulfonate

To a solution of ((R)-3-benzyl-5-(hydroxymethyl)imidazolidin-1-yl)((R)-1-tritylaziridin-2-yl)methanone (3.7 g, 7.35 mmol, 1 equiv) in DCM (30 mL) at 0° C. was added NEt₃ (2.23 g, 22.04 mmol, 3.07 mL, 3 eq), followed by dropwise addition of a solution of MsCl (1.68 g, 14.69 mmol, 1.14 mL, 2 eq) in DCM (7 mL). After 1 hr the reaction was quenched with H₂O (40 mL) and extracted with DCM (3×50 mL). The combined phase was washed with sat. aq. NaCl (25 mL), dried over Na₂SO₄, filtered, and concentrated under reduced pressure to afford ((R)-1-benzyl-3-((R)-1-tritylaziridine-2-carbonyl)imidazolidin-4-yl)methyl methanesulfonate (4.41 g, crude) as a yellow solid which was used without further purification.

### Step 8: Synthesis of 2-((S)-1-benzyl-3-((R)-1-tritylaziridine-2-carbonyl)imidazolidin-4-yl)acetonitrile

To a solution of ((R)-1-benzyl-3-((R)-1-tritylaziridine-2-carbonyl)imidazolidin-4-yl)methyl methanesulfonate (4.27 g, 7.34 mmol, 1 equiv) in DMA (42.7 mL) was added NaCN (719.45 mg, 14.68 mmol, 2 equiv), and the mixture was heated to 50° C. After 12 h the reaction was cooled to room temperature and quenched with H₂O (50 mL), then extracted with EtOAc (3×50 mL). The combined organic phase was washed with sat. aq. NaCl (40 mL), dried over Na₂SO₄, filtered, and under reduced pressure. The residue was purified by silica gel column chromatography (10→50% EtOAc/petroleum ether) to afford 2-((S)-1-benzyl-3-((R)-1-tritylaziridine-2-carbonyl)imidazolidin-4-yl)acetonitrile (2.01 g, 53% yield) as a white solid. ¹H NMR (400 MHz, CDCl₃) δ 7.50-7.39 (m, 6H), 7.32 (br dd, J=2.0, 4.9 Hz, 4H), 7.29-7.23 (m, 9H), 7.16 (br dd, J=2.3, 6.7 Hz, 1H), 4.57-4.42 (m, 1H), 3.97 (d, J=5.5 Hz, 1H), 3.76-3.59 (m, 2H), 3.43 (d, J=12.5 Hz, 1H), 3.10-2.89 (m, 3H), 2.77 (dd, J=3.2, 16.7 Hz, 1H), 2.43 (br d, J=1.3 Hz, 1H), 1.56-1.49 (m, 1H), 1.38 (dd, J=1.1, 5.9 Hz, 1H).

### Step 9: Synthesis of 2-((S)-3-((R)-1-tritylaziridine-2-carbonyl)imidazolidin-4-yl)acetonitrile

To a solution of 2-((S)-1-benzyl-3-((R)-1-tritylaziridine-2-carbonyl)imidazolidin-4-yl)acetonitrile (1.7 g, 3.32 mmol, 1 eq) in THF (17 mL) was added Pd/C (1.13 g, 10% purity). The mixture was stirred under H₂ (50 Psi) and heated to 50° C. After 12 h the reaction mixture was cooled to room temperature, filtered through celite, washed with methanol (2×100 mL), and concentrated under reduced pressure. The resulting residue was purified by silica gel column chromatography (5% MeOH/EtOAc) to afford 2-((S)-3-((R)-1-tritylaziridine-2-carbonyl)imidazolidin-4-yl)acetonitrile (164 mg, 10.72% yield) as a white solid. LCMS (ESI): m/z: [M+Na] calcd for C₂₇H₂₆N₄ONa: 445.20; found 445.0.

### Step 10: Synthesis of 2-((S)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)-3-((R)-1-tritylaziridine-2-carbonyl)imidazolidin-4-yl)acetonitrile

To a solution (S)-7-(8-methylnaphthalen-1-yl)-2-((1-methylpyrrolidin-2-yl)methoxy)-5,6,7,8-tetrahydropyrido[3,4-d]pyrimidin-4-yl trifluoromethanesulfonate (347.11 mg, 323.46 μmol, 1 equiv) in DMF (5.2 mL) was added N,N-diisopropylethylamine (83.61 mg, 646.91 μmol, 2 equiv) and 2-((S)-3-((R)-1-tritylaziridine-2-carbonyl)imidazolidin-4-yl)acetonitrile (164 mg, 388.15 μmol, 1.2 equiv). The resulting mixture was heated to 100° C. After 1 h, the reaction was cooled to room temperature, quenched with H₂O (30 mL), and extracted with EtOAc (3×50 mL). The combined organic phase was washed with sat. aq. NaCl (2×10 mL), dried over Na₂SO₄, filtered, and concentrated under reduced pressure. The resulting residue was purified by silica gel column chromatography (50% MeOH/EtOAc) to afford 2-((S)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)-3-((R)-1-tritylaziridine-2-carbonyl)imidazolidin-4-yl)acetonitrile (118 mg, 42% yield) as a white solid. LCMS (ESI) m/z: [M+H] calcd for C₅₁H₅₃N₈O₂: 809.42; found 809.4.

### Step 11: Synthesis of 2-((S)-3-((R)-aziridine-2-carbonyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)imidazolidin-4-yl)acetonitrile

To a solution of 2-((S)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)-3-((R)-1-tritylaziridine-2-carbonyl)imidazolidin-4-yl)acetonitrile (70 mg, 86.53 μmol, 1 equiv) in CHCl₃ (0.35 mL) and MeOH (0.35 mL) at 0° C. was added TFA (394.63 mg, 3.46 mmol, 40 eq). After 30 min, the reaction was warmed to room temperature and quenched with aqueous NaHCO₃ (20 mL), then extracted with DCM (3×25 mL). The combined organic phase was washed with sat. aq. NaCl (2×7 mL), dried over Na₂SO₄, filtered, and concentrated under reduced pressure. The resulting residue was purified by reverse phase chromatography (30→60% MeCN/H₂O, 10 mM NH₄HCO₃) to afford 2-((S)-3-((R)-aziridine-2-carbonyl)-1-(7-(8-methylnaphthalen-1-yl)-2-(((S)-1-methylpyrrolidin-2-yl)methoxy)-5,6,7,8-tetrahydropyrido[3,4-d]pyrimidin-4-yl)imidazolidin-4-yl)acetonitrile (22.41 mg, 46% yield) as a white solid. LCMS (ESI) m/z: [M+H] calcd for C₃₂H₃₉N₈O₂: 567.31; found 567.3. ¹H NMR (400 MHz, CDCl₃) δ 7.68 (dd, J=8.0, 17.8 Hz, 2H), 7.45-7.38 (m, 1H), 7.38-7.32 (m, 1H), 7.26-7.20 (m, 2H), 5.62-5.43 (m, 1H), 5.33 (br d, J=6.8 Hz, 1H), 4.66 (td, J=3.4, 6.8 Hz, 1H), 4.40 (br s, 1H), 4.31-4.09 (m, 2H), 4.01-3.76 (m, 2H), 3.56 (br s, 1H), 3.34-2.97 (m, 4H), 2.95-2.87 (m, 3H), 2.86-2.74 (m, 2H), 2.72-2.39 (m, 4H), 2.38-2.19 (m, 1H), 2.16-1.68 (m, 5H), 1.59 (br s, 3H), 1.43 (br s, 1H).

### Examples 188 to 216—Synthesis of Exemplary Compounds

The following table of compounds were prepared using the aforementioned methods or variations thereof, as is known to those of skill in the art.

TABLE 3b
Exemplary Compounds Prepared by Methods of the Present Invention

| | | Molecular | Calculated | Observed MW |
|---|---|---|---|---|
| Ex. # | Structure | Formula | MW | LCMS (ESI) m/z |
| 188 | 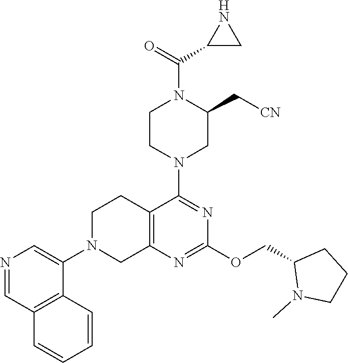 [SMILES: CN1CCC[C@H]1COc1nc2c(c(N3CCN(C(=O)[C@H]4CN4)[C@@H](CC#N)C3)n1)CCN(c1cncc3ccccc13)C2] | C₃₁H₃₇N₉O₂ | [M + H] = 568.32 | [M + H] = 568.3 |
| 189 | 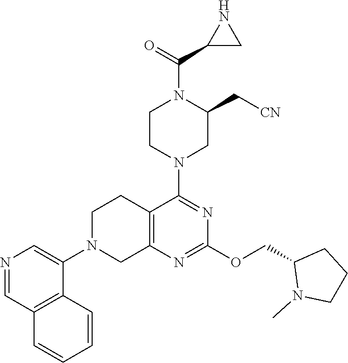 [SMILES: CN1CCC[C@H]1COc1nc2c(c(N3CCN(C(=O)[C@@H]4CN4)[C@@H](CC#N)C3)n1)CCN(c1cncc3ccccc13)C2] | C₃₁H₃₇N₉O₂ | [M + H] = 568.32 | [M + H] = 568.1 |
| 190 | 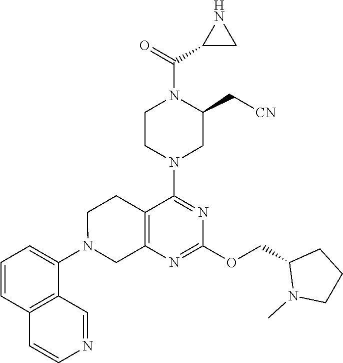 [SMILES: CN1CCC[C@H]1COc1nc2c(c(N3CCN(C(=O)[C@H]4CN4)[C@@H](CC#N)C3)n1)CCN(c1cccc3ccncc13)C2] | C₃₁H₃₇N₉O₂ | [M + H] = 568.32 | [M + H] = 568.4 |
| 191 | 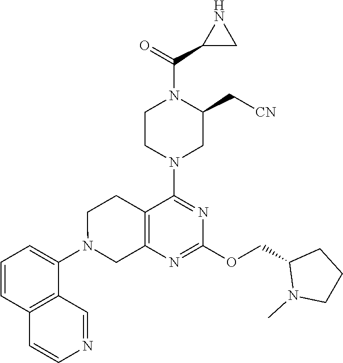 [SMILES: CN1CCC[C@H]1COc1nc2c(c(N3CCN(C(=O)[C@@H]4CN4)[C@@H](CC#N)C3)n1)CCN(c1cccc3ccncc13)C2] | C₃₁H₃₇N₉O₂ | [M + H] = 568.32 | [M + H] = 568.3 |
| 192 | 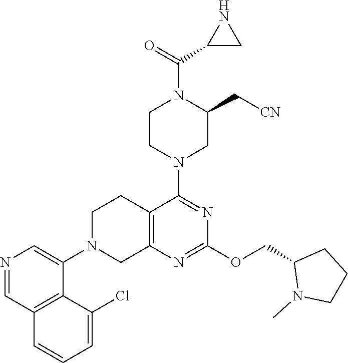 [SMILES: CN1CCC[C@H]1COc1nc2c(c(N3CCN(C(=O)[C@H]4CN4)[C@@H](CC#N)C3)n1)CCN(c1cncc3cccc(Cl)c13)C2] | C₃₁H₃₆ClN₉O₂ | [M + H] = 602.28 | [M + H] = 602.3 |
| 193 | 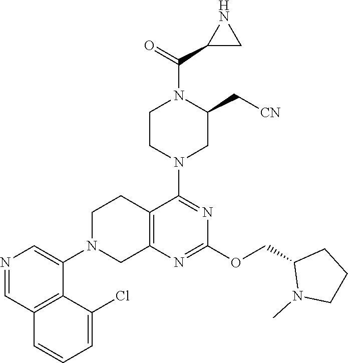 [SMILES: CN1CCC[C@H]1COc1nc2c(c(N3CCN(C(=O)[C@@H]4CN4)[C@@H](CC#N)C3)n1)CCN(c1cncc3cccc(Cl)c13)C2] | C₃₁H₃₆ClN₉O₂ | [M + H] = 602.28 | [M + H] = 602.3 |
| 194 | 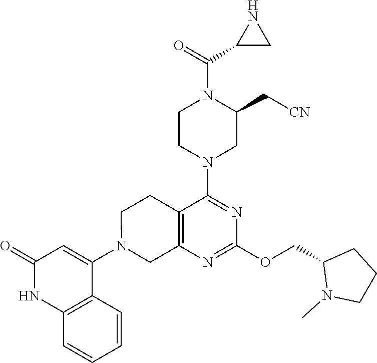 [SMILES: CN1CCC[C@H]1COc1nc2c(c(N3CCN(C(=O)[C@H]4CN4)[C@@H](CC#N)C3)n1)CCN(c1cc(=O)[nH]c3ccccc13)C2] | C₃₁H₃₇N₉O₃ | [M + H] = 584.31 | [M + H] = 584.3 |
| 195 | 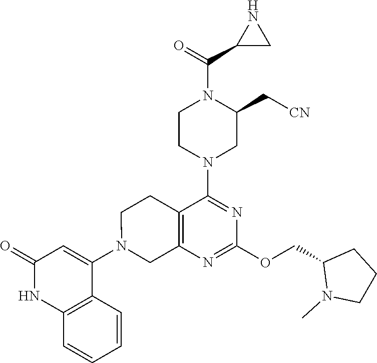 [SMILES: CN1CCC[C@H]1COc1nc2c(c(N3CCN(C(=O)[C@@H]4CN4)[C@@H](CC#N)C3)n1)CCN(c1cc(=O)[nH]c3ccccc13)C2] | C₃₁H₃₇N₉O₃ | [M + H] = 584.31 | [M + H] = 584.3 |
| 196 | 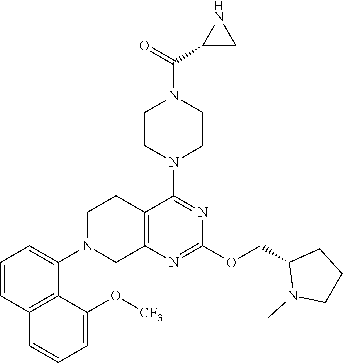 [SMILES: CN1CCC[C@H]1COc1nc2c(c(N3CCN(C(=O)[C@H]4CN4)CC3)n1)CCN(c1cccc3cccc(OC(F)(F)F)c13)C2] | C₃₁H₃₆F₃N₇O₃ | [M + H] = 612.29 | [M + H] = 612.1 |
| 197 | 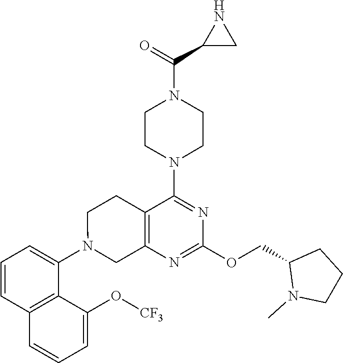 [SMILES: CN1CCC[C@H]1COc1nc2c(c(N3CCN(C(=O)[C@@H]4CN4)CC3)n1)CCN(c1cccc3cccc(OC(F)(F)F)c13)C2] | C₃₁H₃₆F₃N₇O₃ | [M + H] = 612.29 | [M + H] = 612.1 |
| 198 | 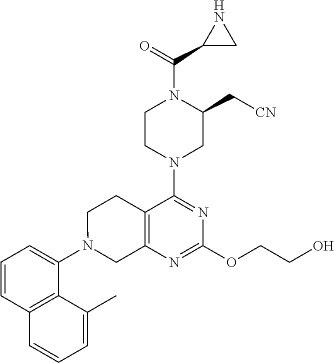 [SMILES: Cc1cccc2cccc(N3CCc4c(nc(OCCO)nc4N4CCN(C(=O)[C@@H]5CN5)[C@@H](CC#N)C4)C3)c12] | C₂₉H₃₃N₇O₃ | [M + H] = 528.27 | [M + H] = 528.3 |
| 199 | 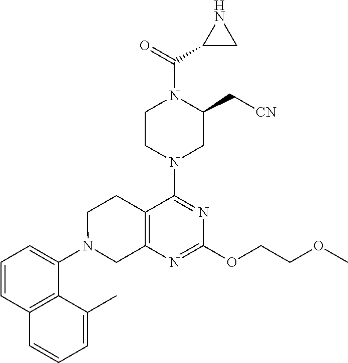 [SMILES: COCCOc1nc2c(c(N3CCN(C(=O)[C@H]4CN4)[C@@H](CC#N)C3)n1)CCN(c1cccc3cccc(C)c13)C2] | C₃₀H₃₅N₇O₃ | [M + H] = 542.29 | [M + H] = 542.3 |
| 200 | 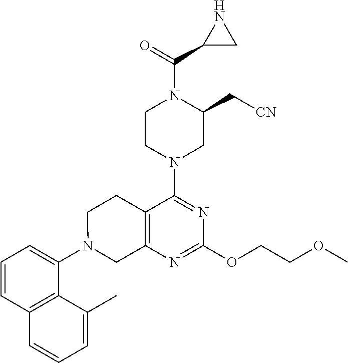 [SMILES: COCCOc1nc2c(c(N3CCN(C(=O)[C@@H]4CN4)[C@@H](CC#N)C3)n1)CCN(c1cccc3cccc(C)c13)C2] | C₃₀H₃₅N₇O₃ | [M + H] = 542.29 | [M + H] = 542.1 |
| 201 | 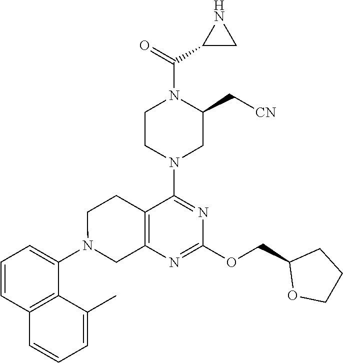 [SMILES: Cc1cccc2cccc(N3CCc4c(nc(OC[C@H]5CCCO5)nc4N4CCN(C(=O)[C@H]5CN5)[C@@H](CC#N)C4)C3)c12] | C₃₂H₃₇N₇O₃ | [M + H] = 568.31 | [M + H] = 568.3 |
| 202 | 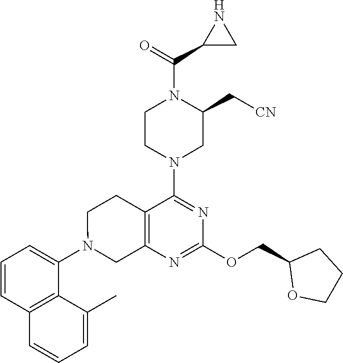 [SMILES: Cc1cccc2cccc(N3CCc4c(nc(OC[C@H]5CCCO5)nc4N4CCN(C(=O)[C@@H]5CN5)[C@@H](CC#N)C4)C3)c12] | C₃₂H₃₇N₇O₃ | [M + H] = 568.31 | [M + H] = 568.3 |
| 203 | 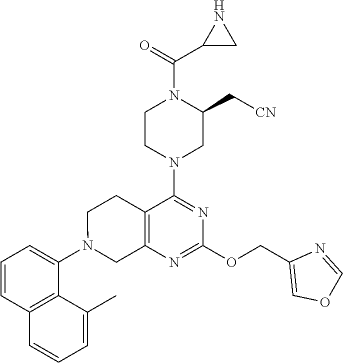 [SMILES: Cc1cccc2cccc(N3CCc4c(nc(OCc5cocn5)nc4N4CCN(C(=O)C5CN5)[C@@H](CC#N)C4)C3)c12] | C₃₁H₃₂N₈O₃ | [M + H] = 565.27 | [M + H] = 565.3 |
| 204 | 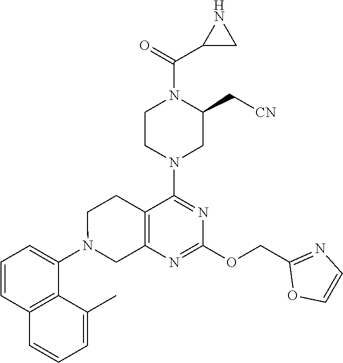 [SMILES: Cc1cccc2cccc(N3CCc4c(nc(OCc5ncco5)nc4N4CCN(C(=O)C5CN5)[C@@H](CC#N)C4)C3)c12] | C₃₁H₃₂N₈O₃ | [M + H] = 565.27 | [M + H] = 565.3 |
| 205 | 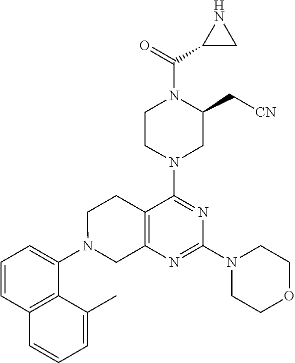 [SMILES: Cc1cccc2cccc(N3CCc4c(nc(N5CCOCC5)nc4N4CCN(C(=O)[C@H]5CN5)[C@@H](CC#N)C4)C3)c12] | C₃₁H₃₆N₈O₂ | [M + H] = 553.31 | [M + H] = 553.2 |
| 206 | 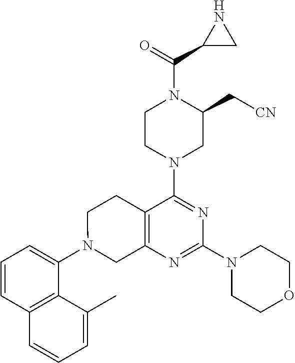 [SMILES: Cc1cccc2cccc(N3CCc4c(nc(N5CCOCC5)nc4N4CCN(C(=O)[C@@H]5CN5)[C@@H](CC#N)C4)C3)c12] | C₃₁H₃₆N₈O₂ | [M + H] = 553.31 | [M + H] = 553.3 |
| 207 | 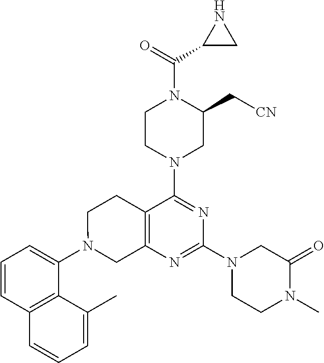 [SMILES: Cc1cccc2cccc(N3CCc4c(nc(N5CCN(C)C(=O)C5)nc4N4CCN(C(=O)[C@H]5CN5)[C@@H](CC#N)C4)C3)c12] | C₃₂H₃₇N₉O₂ | [M + H] = 580.32 | [M + H] = 580.2 |
| 208 | 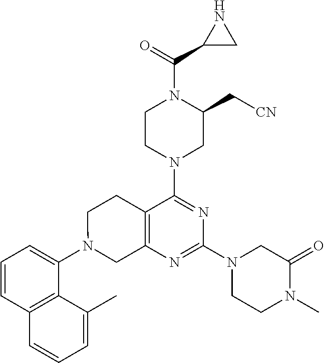 [SMILES: Cc1cccc2cccc(N3CCc4c(nc(N5CCN(C)C(=O)C5)nc4N4CCN(C(=O)[C@@H]5CN5)[C@@H](CC#N)C4)C3)c12] | C₃₂H₃₇N₉O₂ | [M + H] = 580.32 | [M + H] = 580.3 |
| 209 | 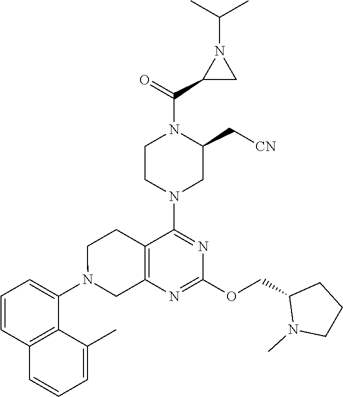 [SMILES: Cc1cccc2cccc(N3CCc4c(nc(OC[C@@H]5CCCN5C)nc4N4CCN(C(=O)[C@@H]5CN5C(C)C)[C@@H](CC#N)C4)C3)c12] | C₃₆H₄₆N₈O₂ | [M + H] = 623.38 | [M + H] = 623.4 |
| 210 | 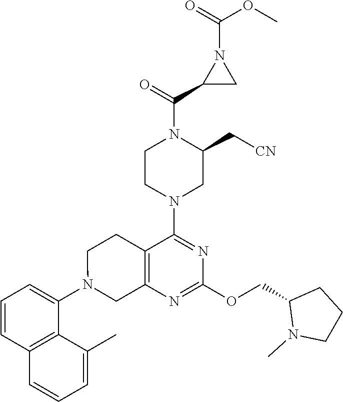 [SMILES: COC(=O)N1C[C@H]1C(=O)N1CCN(c2nc(OC[C@@H]3CCCN3C)nc3c2CCN(c2cccc4cccc(C)c24)C3)C[C@@H]1CC#N] | C₃₅H₄₂N₈O₄ | [M + H] = 639.34 | [M + H] = 639.3 |
| 211 | 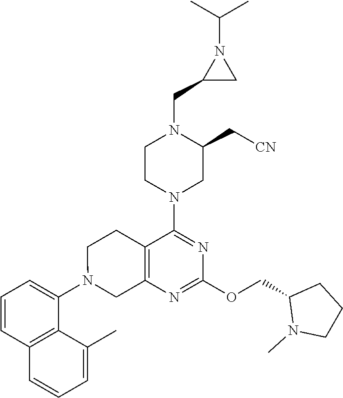 [SMILES: Cc1cccc2cccc(N3CCc4c(nc(OC[C@@H]5CCCN5C)nc4N4CCN(C[C@@H]5CN5C(C)C)[C@@H](CC#N)C4)C3)c12] | C₃₆H₄₈N₈O | [M + H] = 609.41 | [M + H] = 609.4 |
| 212 | 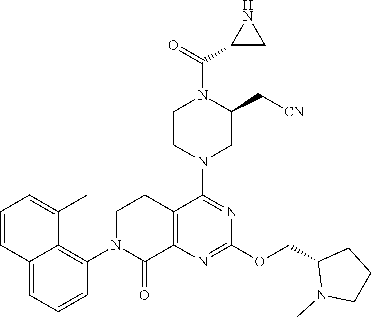 [SMILES: Cc1cccc2cccc(N3CCc4c(nc(OC[C@@H]5CCCN5C)nc4N4CCN(C(=O)[C@H]5CN5)[C@@H](CC#N)C4)C3=O)c12] | C₃₃H₃₈N₈O₃ | [M + H] = 595.32 | [M + H] = 595.2 |
| 213 | 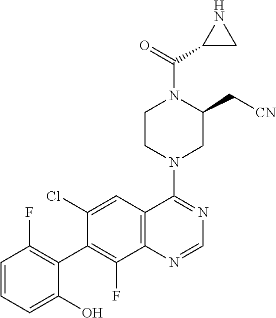 [SMILES: N#CC[C@H]1CN(c2ncnc3c(F)c(-c4c(O)cccc4F)c(Cl)cc23)CCN1C(=O)[C@H]1CN1] | C₂₃H₁₉ClF₂N₆O₂ | [M + H] = 485.13 | [M + H] = 485.1 |
| 214 | 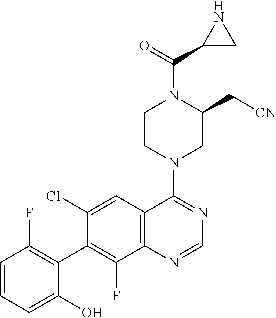 [SMILES: N#CC[C@H]1CN(c2ncnc3c(F)c(-c4c(O)cccc4F)c(Cl)cc23)CCN1C(=O)[C@@H]1CN1] | C₂₃H₁₉ClF₂N₆O₂ | [M + H] = 485.13 | [M + H] = 485.1 |
| 215* | 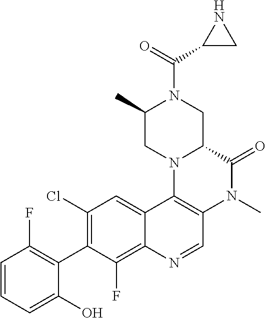 [SMILES: C[C@@H]1CN2c3c(cnc4c(F)c(-c5c(O)cccc5F)c(Cl)cc34)N(C)C(=O)[C@H]2CN1C(=O)[C@H]1CN1] | C₂₅H₂₂ClF₂N₅O₃ | [M + H] = 514.15 | [M + H] = 514.2 |
| 216* | 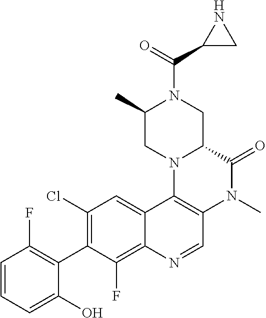 [SMILES: C[C@@H]1CN2c3c(cnc4c(F)c(-c5c(O)cccc5F)c(Cl)cc34)N(C)C(=O)[C@H]2CN1C(=O)[C@@H]1CN1] | C₂₅H₂₂ClF₂N₅O₃ | [M + H] = 514.15 | [M + H] = 514.2 |

*Prepared according to reaction scheme 1, amine intermediate prepared according to procedures described in WO2019110751.
Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention.

### Example 219—Cross-Linking of Ras Proteins with Compounds of the Invention to Form Conjugates

### Protocol: K-Ras G12D(GDP) Cross-Linking Assay

Note: while the following protocol outlines the procedure for K-Ras G12D(GDP), a person of skill in the art may substitute other Ras proteins, and may also substitute a non-hydrolyzable GTP analog for GDP to study GTP bound Ras protein.

GDP-loaded K-Ras (1-169) G12D, C51S, C80L, C118S and GDP-loaded K-Ras (1-169) C51S, C80L, C118S were adjusted to 50 μM in K-Ras assay buffer (12.5 mM HEPES, 75 mM NaCl and 1 mM MgCl₂ at pH 7.4). A 5 μL aliquot of each protein solution was added to each well of a 96-well microplate containing 40 μL of assay buffer. Initial compound stocks were prepared in DMSO at 100 times their final assay concentration. Compounds were then diluted 10-fold into K-Ras assay buffer to 10 times their final concentration. A 5 μL aliquot of each diluted compound solution was added to each protein solution in the 96-well microplate to initiate the reaction, which then proceeded at room temperature. Typical final compound concentrations were 2, 10, and 25 μM. At each time point, the reactions were analyzed immediately or quenched with 5 μL of a 5% formic acid solution and kept at 4° C. until analysis. Typical assay endpoints were 1 and 24 h.

Data collection took place on an Agilent 6230 TOF Mass Spectrometer. Complete reactions were injected onto a C4 reverse-phase column to separate protein from buffer components prior to entering the mass spectrometer. The proteins were eluted from the column by increasing acetonitrile fraction in the mobile phase and fed directly into the mass analyzer. Initial analysis of raw data took place in Agilent MassHunter BioConfirm software and consisted of deconvolution of multiple protein charge states with the maximum entropy algorithm, with a mass step of 1 Da. The heights of all deconvoluted protein masses were exported for further data analysis. The percent modification of each protein was then determined by calculating the peak height of the covalently modified K-Ras species as a percentage of the sum total of K-Ras protein peak height.

Comparable procedures were run with other Ras proteins to produce the results seen in Tables 4 and 5.

TABLE 4
Conjugate Formation Data

| | | G12D x- | WT x- | G12S x- |
|---|---|---|---|---|
| Ex. # | Structure | linking (%) | linking (%) | linking (%) |
| 1 | 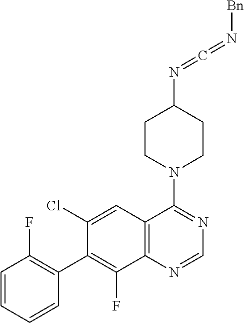 [SMILES: Fc1ccccc1-c1c(Cl)cc2c(N3CCC(N=C=NCc4ccccc4)CC3)ncnc2c1F] | + | + | + |
| 2 | 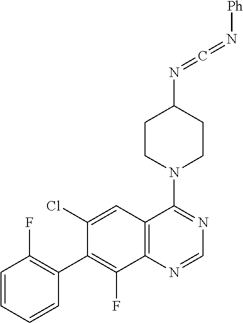 [SMILES: Fc1ccccc1-c1c(Cl)cc2c(N3CCC(N=C=Nc4ccccc4)CC3)ncnc2c1F] | + | + | + |
| 3 | 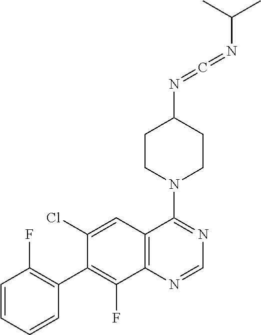 [SMILES: CC(C)N=C=NC1CCN(c2ncnc3c(F)c(-c4ccccc4F)c(Cl)cc23)CC1] | + | + | + |
| 4 | 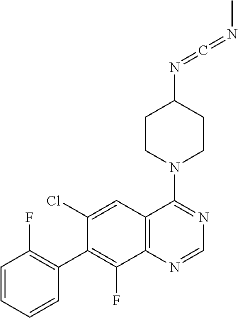 [SMILES: CN=C=NC1CCN(c2ncnc3c(F)c(-c4ccccc4F)c(Cl)cc23)CC1] | + | + | ++ |
| 5 | 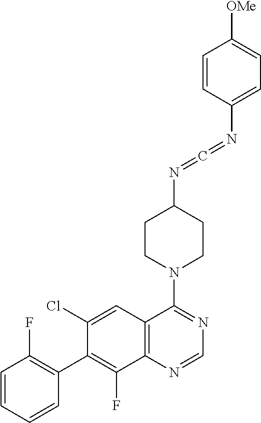 [SMILES: COc1ccc(N=C=NC2CCN(c3ncnc4c(F)c(-c5ccccc5F)c(Cl)cc34)CC2)cc1] | 0 | 0 | 0 |
| 6 | 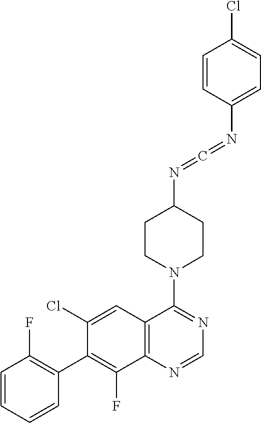 [SMILES: Fc1ccccc1-c1c(Cl)cc2c(N3CCC(N=C=Nc4ccc(Cl)cc4)CC3)ncnc2c1F] | 0 | 0 | 0 |
| 7 | 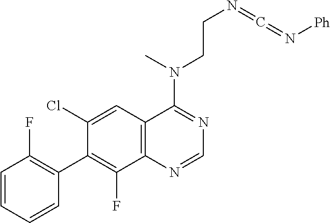 [SMILES: CN(CCN=C=Nc1ccccc1)c1ncnc2c(F)c(-c3ccccc3F)c(Cl)cc12] | + | + | + |
| 8 | 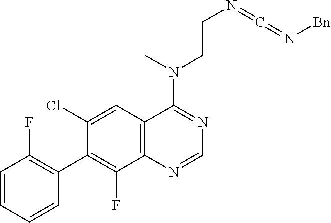 [SMILES: CN(CCN=C=NCc1ccccc1)c1ncnc2c(F)c(-c3ccccc3F)c(Cl)cc12] | + | + | + |
| 9 | 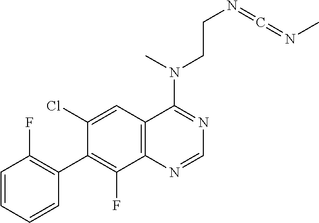 [SMILES: CN=C=NCCN(C)c1ncnc2c(F)c(-c3ccccc3F)c(Cl)cc12] | + | + | + |
| 10 | 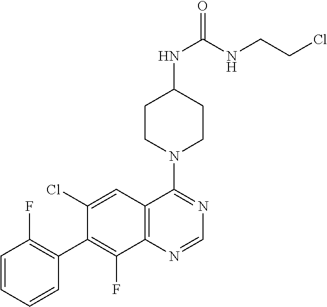 [SMILES: O=C(NCCCl)NC1CCN(c2ncnc3c(F)c(-c4ccccc4F)c(Cl)cc23)CC1] | + | + | + |
| 11 | 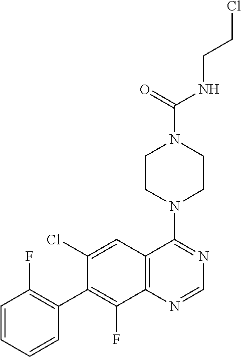 [SMILES: O=C(NCCCl)N1CCN(c2ncnc3c(F)c(-c4ccccc4F)c(Cl)cc23)CC1] | ND | ND | ND |
| 12 | 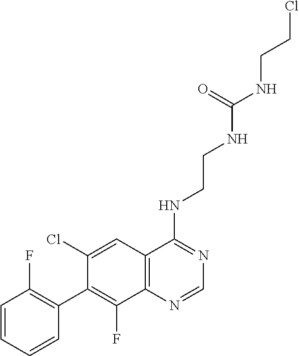 [SMILES: O=C(NCCCl)NCCNc1ncnc2c(F)c(-c3ccccc3F)c(Cl)cc12] | 0 | 0 | 0 |
| 13 | 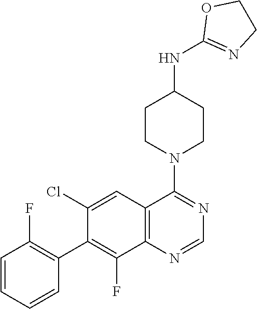 [SMILES: Fc1ccccc1-c1c(Cl)cc2c(N3CCC(NC4=NCCO4)CC3)ncnc2c1F] | + | + | + |
| 14 | 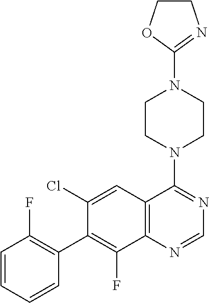 [SMILES: Fc1ccccc1-c1c(Cl)cc2c(N3CCN(C4=NCCO4)CC3)ncnc2c1F] | + | + | + |
| 15 | 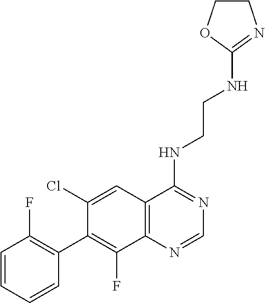 [SMILES: Fc1ccccc1-c1c(Cl)cc2c(NCCNC3=NCCO3)ncnc2c1F] | + | + | + |
| 16 | 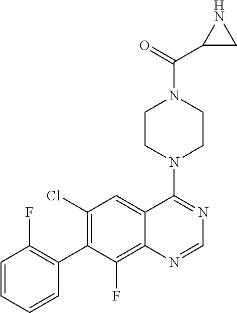 [SMILES: O=C(C1CN1)N1CCN(c2ncnc3c(F)c(-c4ccccc4F)c(Cl)cc23)CC1] | + | + | + |
| 17 | 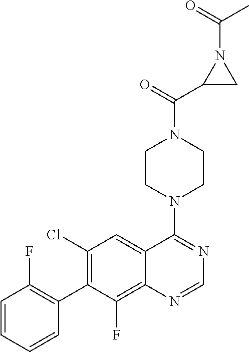 [SMILES: CC(=O)N1CC1C(=O)N1CCN(c2ncnc3c(F)c(-c4ccccc4F)c(Cl)cc23)CC1] | + | + | + |
| 18 | 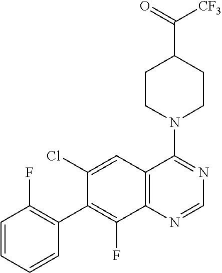 [SMILES: O=C(C1CCN(c2ncnc3c(F)c(-c4ccccc4F)c(Cl)cc23)CC1)C(F)(F)F] | 0 | 0 | 0 |
| 19 | 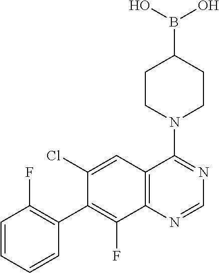 [SMILES: OB(O)C1CCN(c2ncnc3c(F)c(-c4ccccc4F)c(Cl)cc23)CC1] | 0 | 0 | 0 |
| 20 | 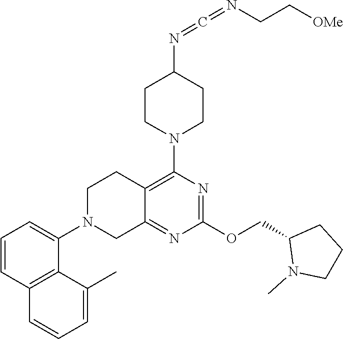 [SMILES: COCCN=C=NC1CCN(c2nc(OC[C@@H]3CCCN3C)nc3c2CCN(c2cccc4cccc(C)c24)C3)CC1] | +++ | ++ | ++ |
| 21 | 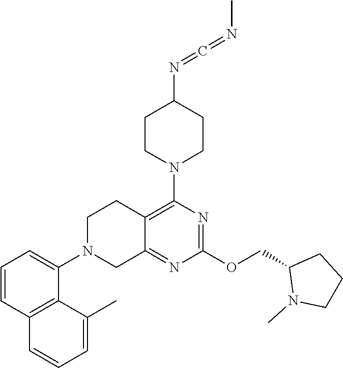 [SMILES: CN=C=NC1CCN(c2nc(OC[C@@H]3CCCN3C)nc3c2CCN(c2cccc4cccc(C)c24)C3)CC1] | +++ | ++ | ++ |
| 22 | 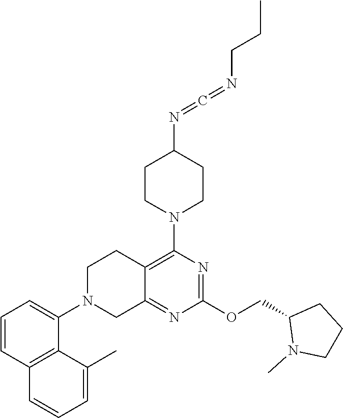 [SMILES: CCCN=C=NC1CCN(c2nc(OC[C@@H]3CCCN3C)nc3c2CCN(c2cccc4cccc(C)c24)C3)CC1] | +++ | ++ | ++ |
| 23 | 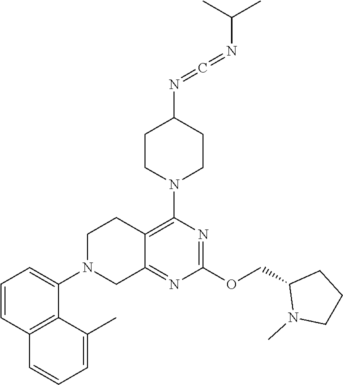 [SMILES: Cc1cccc2cccc(N3CCc4c(nc(OC[C@@H]5CCCN5C)nc4N4CCC(N=C=NC(C)C)CC4)C3)c12] | ++ | + | ++ |
| 24 | 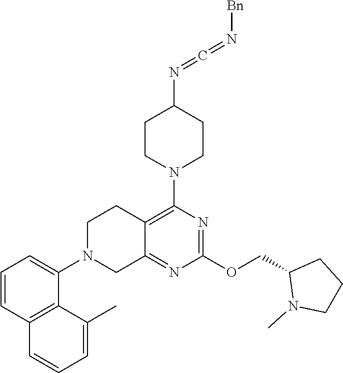 [SMILES: Cc1cccc2cccc(N3CCc4c(nc(OC[C@@H]5CCCN5C)nc4N4CCC(N=C=NCc5ccccc5)CC4)C3)c12] | + | + | + |
| 25 | 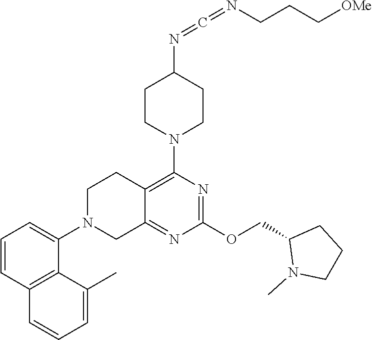 [SMILES: COCCCN=C=NC1CCN(c2nc(OC[C@@H]3CCCN3C)nc3c2CCN(c2cccc4cccc(C)c24)C3)CC1] | +++ | ++ | ++ |
| 26 | 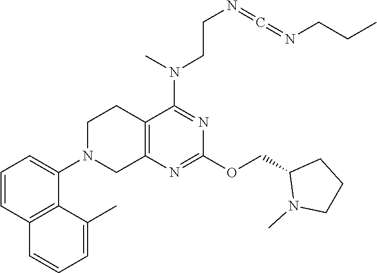 [SMILES: CCCN=C=NCCN(C)c1nc(OC[C@@H]2CCCN2C)nc2c1CCN(c1cccc3cccc(C)c13)C2] | +++ | ++ | ++ |
| 27 | 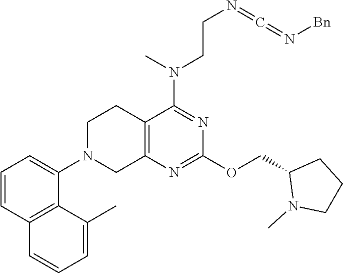 [SMILES: Cc1cccc2cccc(N3CCc4c(nc(OC[C@@H]5CCCN5C)nc4N(C)CCN=C=NCc4ccccc4)C3)c12] | ++ | + | ++ |
| 28 | 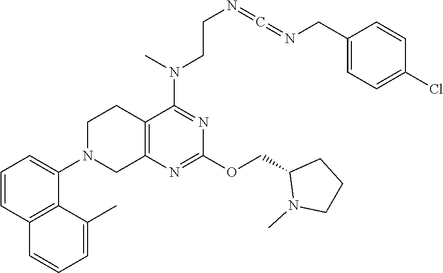 [SMILES: Cc1cccc2cccc(N3CCc4c(nc(OC[C@@H]5CCCN5C)nc4N(C)CCN=C=NCc4ccc(Cl)cc4)C3)c12] | ++ | + | ++ |
| 29 | 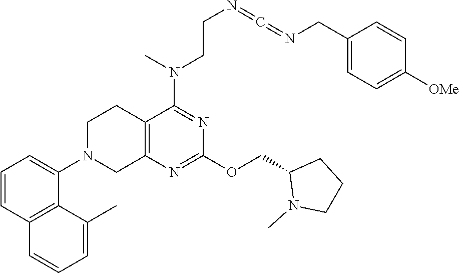 [SMILES: COc1ccc(CN=C=NCCN(C)c2nc(OC[C@@H]3CCCN3C)nc3c2CCN(c2cccc4cccc(C)c24)C3)cc1] | +++ | ++ | ++ |
| 30 | 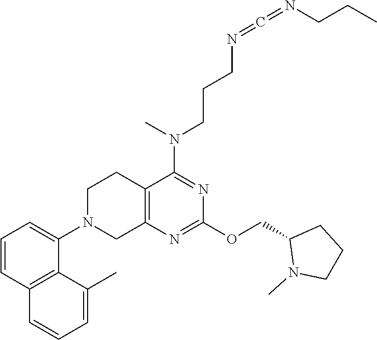 [SMILES: CCCN=C=NCCCN(C)c1nc(OC[C@@H]2CCCN2C)nc2c1CCN(c1cccc3cccc(C)c13)C2] | +++ | ++ | ++ |
| 31 | 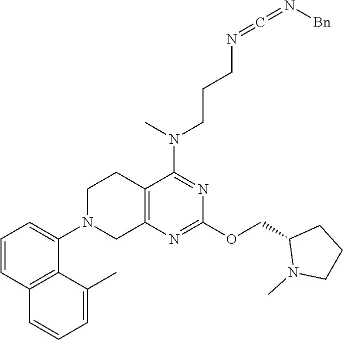 [SMILES: Cc1cccc2cccc(N3CCc4c(nc(OC[C@@H]5CCCN5C)nc4N(C)CCCN=C=NCc4ccccc4)C3)c12] | +++ | ++ | ++ |
| 32 | 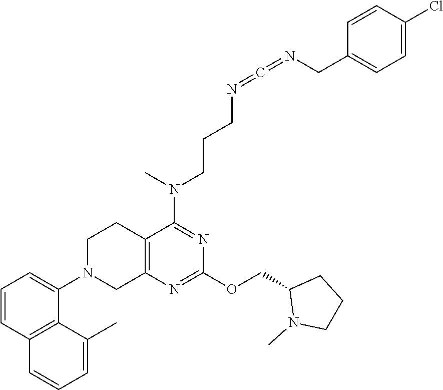 [SMILES: Cc1cccc2cccc(N3CCc4c(nc(OC[C@@H]5CCCN5C)nc4N(C)CCCN=C=NCc4ccc(Cl)cc4)C3)c12] | + | + | + |
| 33 | 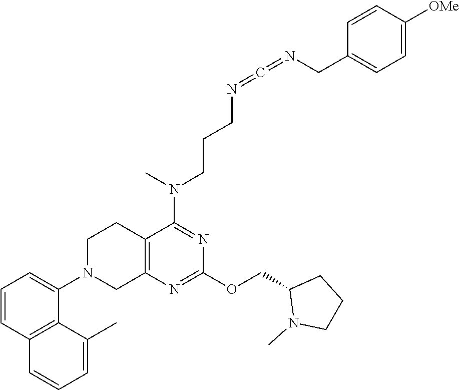 [SMILES: COc1ccc(CN=C=NCCCN(C)c2nc(OC[C@@H]3CCCN3C)nc3c2CCN(c2cccc4cccc(C)c24)C3)cc1] | +++ | ++ | ++ |
| 34 | 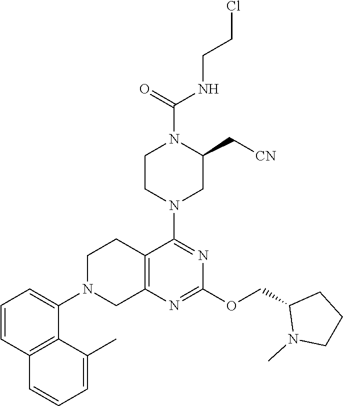 [SMILES: Cc1cccc2cccc(N3CCc4c(nc(OC[C@@H]5CCCN5C)nc4N4CCN(C(=O)NCCCl)[C@@H](CC#N)C4)C3)c12] | + | + | + |
| 35 | 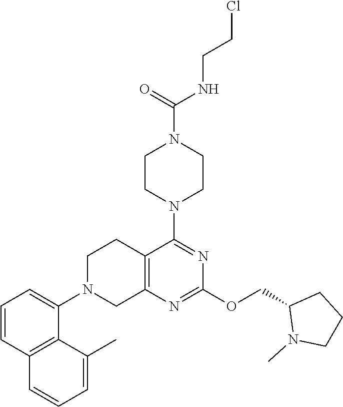 [SMILES: Cc1cccc2cccc(N3CCc4c(nc(OC[C@@H]5CCCN5C)nc4N4CCN(C(=O)NCCCl)CC4)C3)c12] | ND | ND | ND |
| 36 | 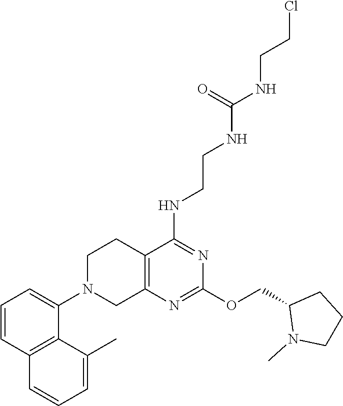 [SMILES: Cc1cccc2cccc(N3CCc4c(nc(OC[C@@H]5CCCN5C)nc4NCCNC(=O)NCCCl)C3)c12] | + | + | + |
| 37 | 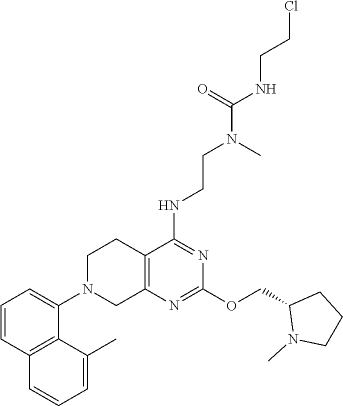 [SMILES: Cc1cccc2cccc(N3CCc4c(nc(OC[C@@H]5CCCN5C)nc4NCCN(C)C(=O)NCCCl)C3)c12] | 0 | 0 | 0 |
| 38 | 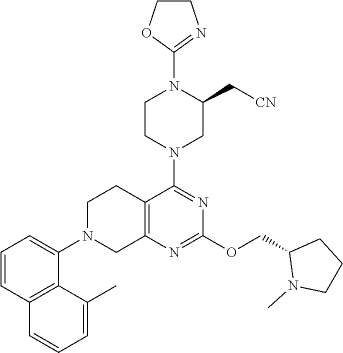 [SMILES: Cc1cccc2cccc(N3CCc4c(nc(OC[C@@H]5CCCN5C)nc4N4CCN(C5=NCCO5)[C@@H](CC#N)C4)C3)c12] | 0 | 0 | 0 |
| 39 | 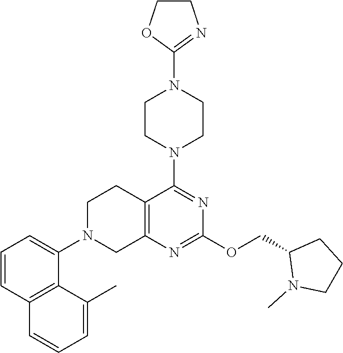 [SMILES: Cc1cccc2cccc(N3CCc4c(nc(OC[C@@H]5CCCN5C)nc4N4CCN(C5=NCCO5)CC4)C3)c12] | 0 | 0 | 0 |
| 40 | 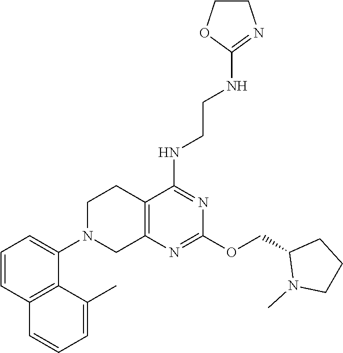 [SMILES: Cc1cccc2cccc(N3CCc4c(nc(OC[C@@H]5CCCN5C)nc4NCCNC4=NCCO4)C3)c12] | + | + | + |
| 41 | 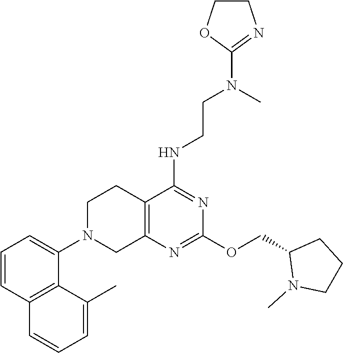 [SMILES: Cc1cccc2cccc(N3CCc4c(nc(OC[C@@H]5CCCN5C)nc4NCCN(C)C4=NCCO4)C3)c12] | 0 | 0 | 0 |
| 42 | 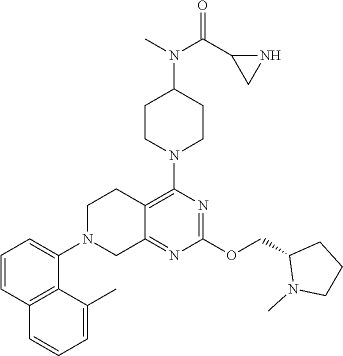 [SMILES: Cc1cccc2cccc(N3CCc4c(nc(OC[C@@H]5CCCN5C)nc4N4CCC(N(C)C(=O)C5CN5)CC4)C3)c12] | + | + | + |
| 43 | 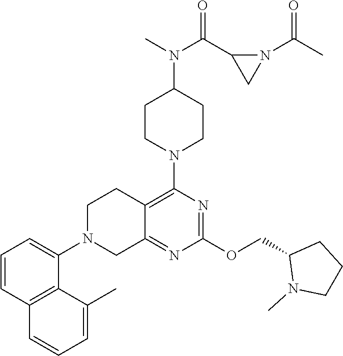 [SMILES: CC(=O)N1CC1C(=O)N(C)C1CCN(c2nc(OC[C@@H]3CCCN3C)nc3c2CCN(c2cccc4cccc(C)c24)C3)CC1] | + | + | + |
| 44 | 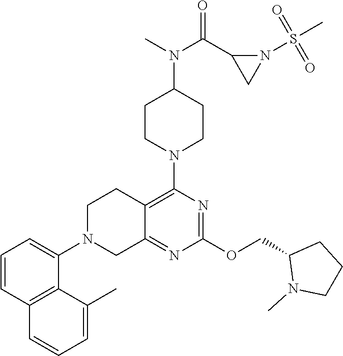 [SMILES: Cc1cccc2cccc(N3CCc4c(nc(OC[C@@H]5CCCN5C)nc4N4CCC(N(C)C(=O)C5CN5S(C)(=O)=O)CC4)C3)c12] | + | + | + |
| 45 | 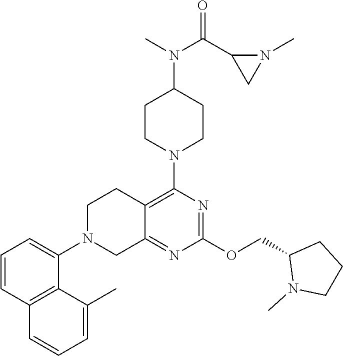 [SMILES: Cc1cccc2cccc(N3CCc4c(nc(OC[C@@H]5CCCN5C)nc4N4CCC(N(C)C(=O)C5CN5C)CC4)C3)c12] | + | + | + |
| 46 | 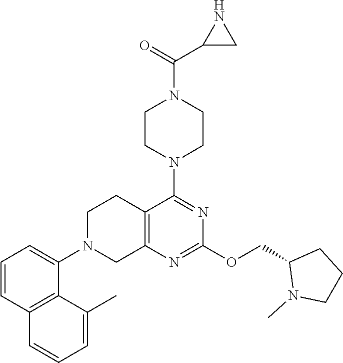 [SMILES: Cc1cccc2cccc(N3CCc4c(nc(OC[C@@H]5CCCN5C)nc4N4CCN(C(=O)C5CN5)CC4)C3)c12] | + | + | + |
| 47 | 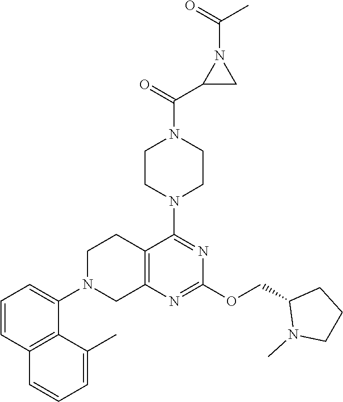 [SMILES: CC(=O)N1CC1C(=O)N1CCN(c2nc(OC[C@@H]3CCCN3C)nc3c2CCN(c2cccc4cccc(C)c24)C3)CC1] | + | + | + |
| 48 | 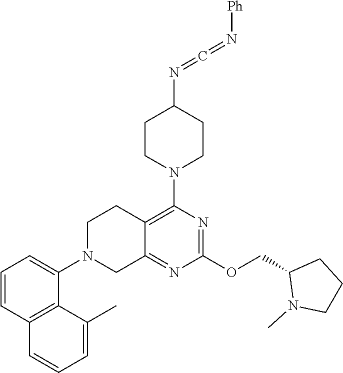 [SMILES: Cc1cccc2cccc(N3CCc4c(nc(OC[C@@H]5CCCN5C)nc4N4CCC(N=C=Nc5ccccc5)CC4)C3)c12] | + | + | + |
| 49 | 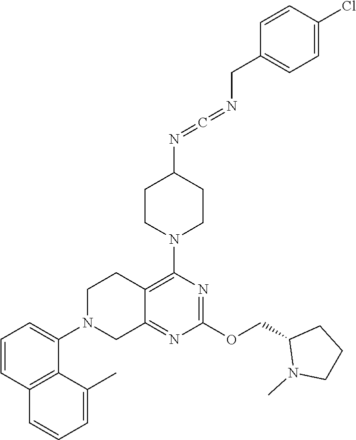 [SMILES: Cc1cccc2cccc(N3CCc4c(nc(OC[C@@H]5CCCN5C)nc4N4CCC(N=C=NCc5ccc(Cl)cc5)CC4)C3)c12] | + | + | + |
| 50 | 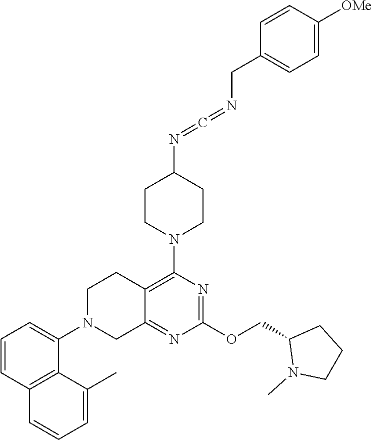 [SMILES: COc1ccc(CN=C=NC2CCN(c3nc(OC[C@@H]4CCCN4C)nc4c3CCN(c3cccc5cccc(C)c35)C4)CC2)cc1] | + | + | + |
| 51 | 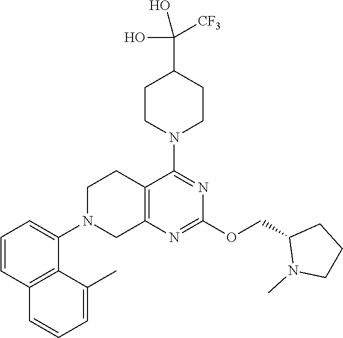 [SMILES: Cc1cccc2cccc(N3CCc4c(nc(OC[C@@H]5CCCN5C)nc4N4CCC(C(O)(O)C(F)(F)F)CC4)C3)c12] | + | + | + |
| 52 | 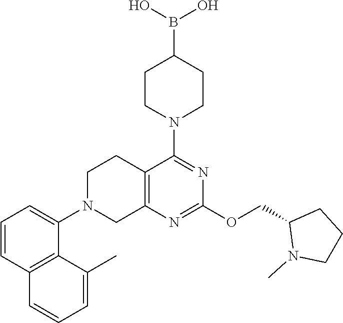 [SMILES: Cc1cccc2cccc(N3CCc4c(nc(OC[C@@H]5CCCN5C)nc4N4CCC(B(O)O)CC4)C3)c12] | + | + | + |
| 53 | 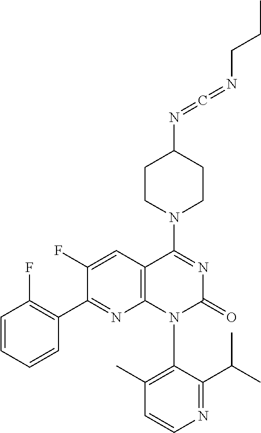 [SMILES: CCCN=C=NC1CCN(c2nc(=O)n(-c3c(C)ccnc3C(C)C)c3nc(-c4ccccc4F)c(F)cc23)CC1] | + | + | + |
| 54 | 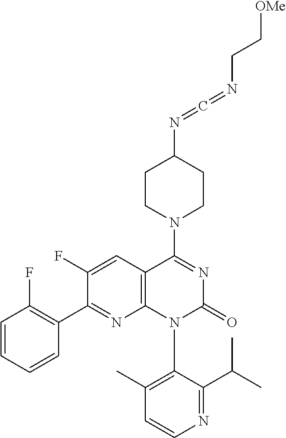 [SMILES: COCCN=C=NC1CCN(c2nc(=O)n(-c3c(C)ccnc3C(C)C)c3nc(-c4ccccc4F)c(F)cc23)CC1] | + | + | + |
| 55 | 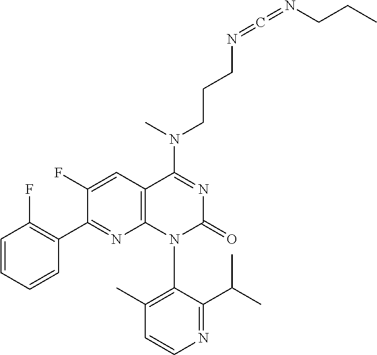 [SMILES: CCCN=C=NCCCN(C)c1nc(=O)n(-c2c(C)ccnc2C(C)C)c2nc(-c3ccccc3F)c(F)cc12] | ++ | ++ | ++ |
| 56 | 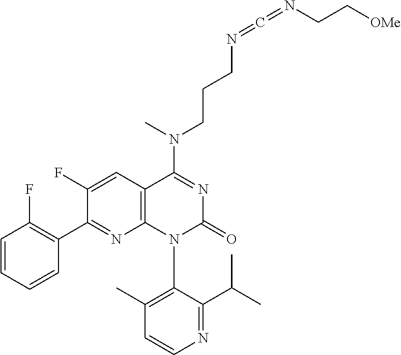 [SMILES: COCCN=C=NCCCN(C)c1nc(=O)n(-c2c(C)ccnc2C(C)C)c2nc(-c3ccccc3F)c(F)cc12] | ++ | ++ | ++ |
| 57 | 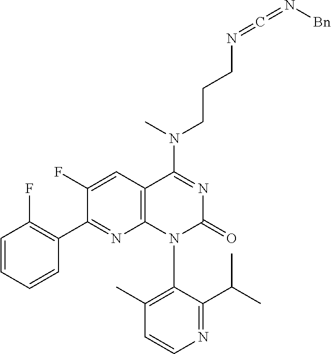 [SMILES: Cc1ccnc(C(C)C)c1-n1c(=O)nc(N(C)CCCN=C=NCc2ccccc2)c2cc(F)c(-c3ccccc3F)nc21] | ++ | + | ++ |
| 58 | 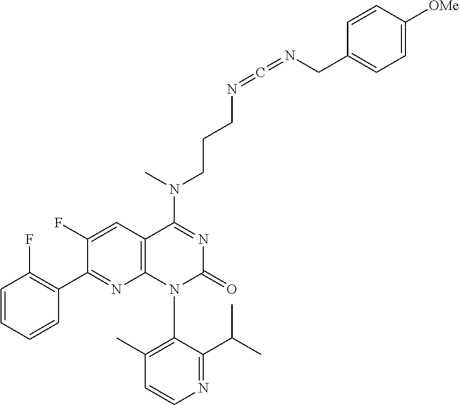 [SMILES: COc1ccc(CN=C=NCCCN(C)c2nc(=O)n(-c3c(C)ccnc3C(C)C)c3nc(-c4ccccc4F)c(F)cc23)cc1] | + | + | ++ |
| 59 | 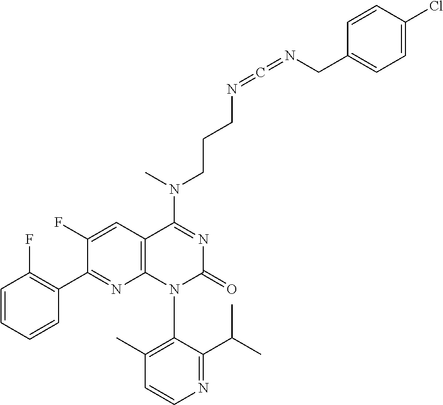 [SMILES: Cc1ccnc(C(C)C)c1-n1c(=O)nc(N(C)CCCN=C=NCc2ccc(Cl)cc2)c2cc(F)c(-c3ccccc3F)nc21] | ND | ND | ND |
| 60 | 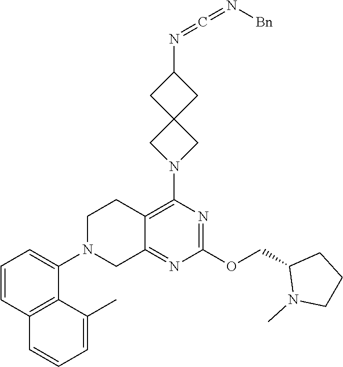 [SMILES: Cc1cccc2cccc(N3CCc4c(nc(OC[C@@H]5CCCN5C)nc4N4CC5(CC(N=C=NCc6ccccc6)C5)C4)C3)c12] | ND | ND | ND |
| 61 | 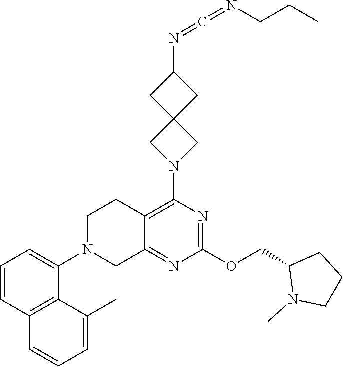 [SMILES: CCCN=C=NC1CC2(C1)CN(c1nc(OC[C@@H]3CCCN3C)nc3c1CCN(c1cccc4cccc(C)c14)C3)C2] | ND | ND | ND |
| 62 | 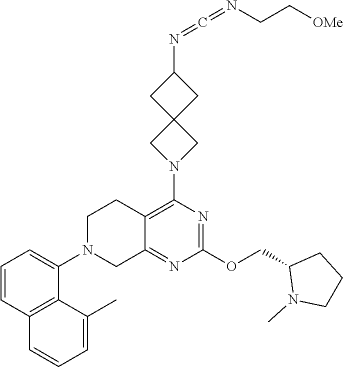 [SMILES: COCCN=C=NC1CC2(C1)CN(c1nc(OC[C@@H]3CCCN3C)nc3c1CCN(c1cccc4cccc(C)c14)C3)C2] | ND | ND | ND |

TABLE 5
Additional Conjugate Formation Data

| | | G12D x- | WT x- | G12S x- |
|---|---|---|---|---|
| Ex. # | Structure | linking (%) | linking (%) | linking (%) |
| 63 | 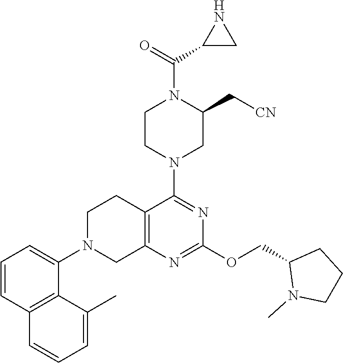 [SMILES: Cc1cccc2cccc(N3CCc4c(nc(OC[C@@H]5CCCN5C)nc4N4CCN(C(=O)[C@H]5CN5)[C@@H](CC#N)C4)C3)c12] | ++ | + | + |
| 64 | 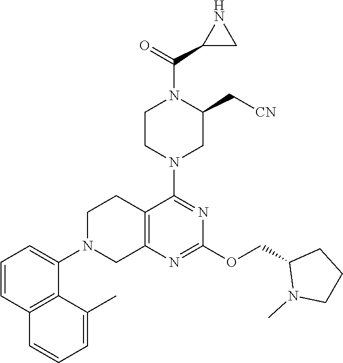 [SMILES: Cc1cccc2cccc(N3CCc4c(nc(OC[C@@H]5CCCN5C)nc4N4CCN(C(=O)[C@@H]5CN5)[C@@H](CC#N)C4)C3)c12] | + | + | + |
| 65 | 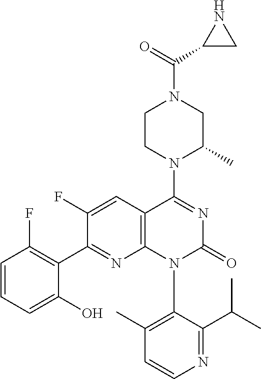 [SMILES: Cc1ccnc(C(C)C)c1-n1c(=O)nc(N2CCN(C(=O)[C@H]3CN3)C[C@@H]2C)c2cc(F)c(-c3c(O)cccc3F)nc21] | + | + | + |
| 66 | 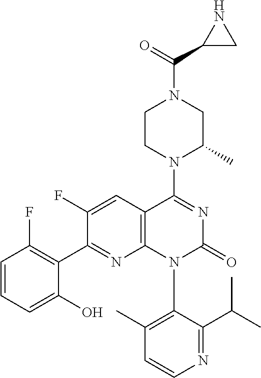 [SMILES: Cc1ccnc(C(C)C)c1-n1c(=O)nc(N2CCN(C(=O)[C@@H]3CN3)C[C@@H]2C)c2cc(F)c(-c3c(O)cccc3F)nc21] | + | + | + |
| 67 | 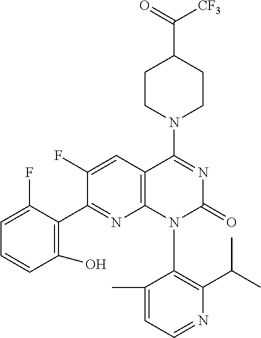 [SMILES: Cc1ccnc(C(C)C)c1-n1c(=O)nc(N2CCC(C(=O)C(F)(F)F)CC2)c2cc(F)c(-c3c(O)cccc3F)nc21] | ND | ND | ND |
| 68 | 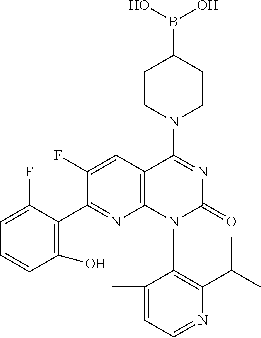 [SMILES: Cc1ccnc(C(C)C)c1-n1c(=O)nc(N2CCC(B(O)O)CC2)c2cc(F)c(-c3c(O)cccc3F)nc21] | ND | ND | ND |
| 69 | 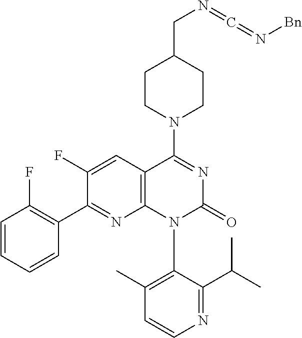 [SMILES: Cc1ccnc(C(C)C)c1-n1c(=O)nc(N2CCC(CN=C=NCc3ccccc3)CC2)c2cc(F)c(-c3ccccc3F)nc21] | + | + | + |
| 70 | 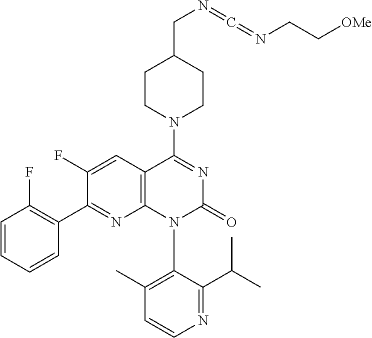 [SMILES: COCCN=C=NCC1CCN(c2nc(=O)n(-c3c(C)ccnc3C(C)C)c3nc(-c4ccccc4F)c(F)cc23)CC1] | ++ | ++ | ++ |
| 71 | 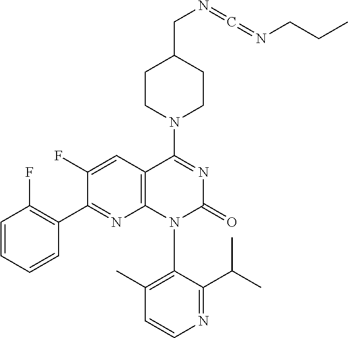 [SMILES: CCCN=C=NCC1CCN(c2nc(=O)n(-c3c(C)ccnc3C(C)C)c3nc(-c4ccccc4F)c(F)cc23)CC1] | ++ | ++ | ++ |
| 72 | 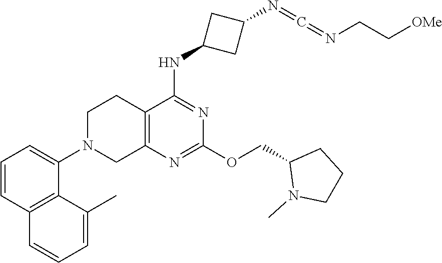 [SMILES: COCCN=C=N[C@H]1C[C@H](Nc2nc(OC[C@@H]3CCCN3C)nc3c2CCN(c2cccc4cccc(C)c24)C3)C1] | +++ | ++ | ++ |
| 73 | 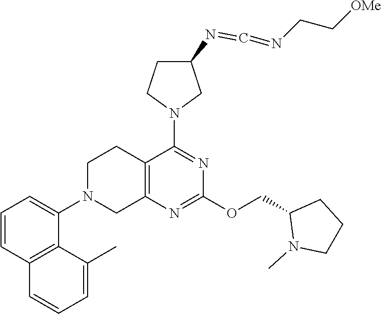 [SMILES: COCCN=C=N[C@@H]1CCN(c2nc(OC[C@@H]3CCCN3C)nc3c2CCN(c2cccc4cccc(C)c24)C3)C1] | +++ | ++ | ++ |
| 74 | 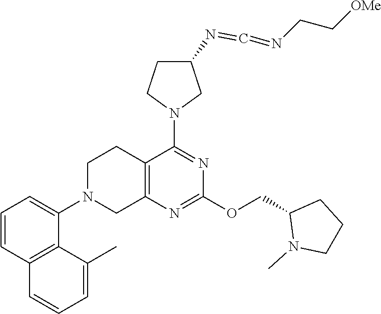 [SMILES: COCCN=C=N[C@H]1CCN(c2nc(OC[C@@H]3CCCN3C)nc3c2CCN(c2cccc4cccc(C)c24)C3)C1] | +++ | ++ | ++ |
| 75 | 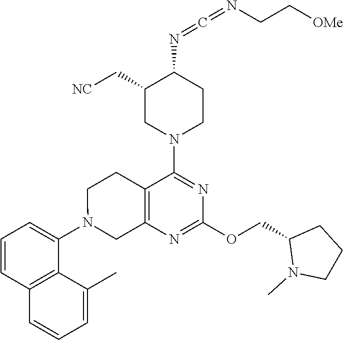 [SMILES: COCCN=C=N[C@@H]1CCN(c2nc(OC[C@@H]3CCCN3C)nc3c2CCN(c2cccc4cccc(C)c24)C3)C[C@@H]1CC#N] | +++ | + | ++ |
| 76 | 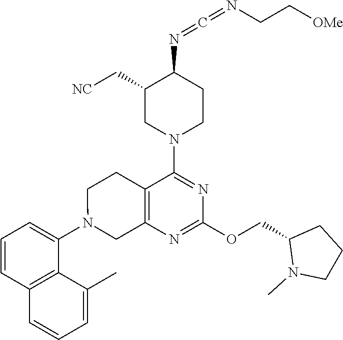 [SMILES: COCCN=C=N[C@H]1CCN(c2nc(OC[C@@H]3CCCN3C)nc3c2CCN(c2cccc4cccc(C)c24)C3)C[C@@H]1CC#N] | +++ | ++ | ND |
| 77 | 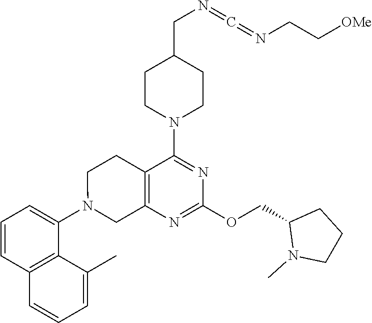 [SMILES: COCCN=C=NCC1CCN(c2nc(OC[C@@H]3CCCN3C)nc3c2CCN(c2cccc4cccc(C)c24)C3)CC1] | +++ | ++ | ++ |
| 78 | 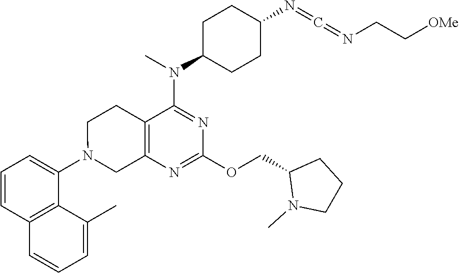 [SMILES: COCCN=C=N[C@H]1CC[C@H](N(C)c2nc(OC[C@@H]3CCCN3C)nc3c2CCN(c2cccc4cccc(C)c24)C3)CC1] | ++ | + | + |
| 79 | 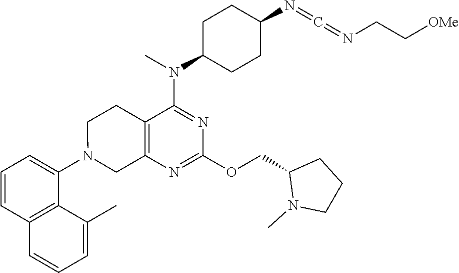 [SMILES: COCCN=C=N[C@H]1CC[C@@H](N(C)c2nc(OC[C@@H]3CCCN3C)nc3c2CCN(c2cccc4cccc(C)c24)C3)CC1] | +++ | ++ | ++ |
| 80 | 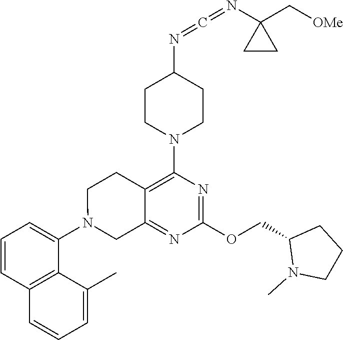 [SMILES: COCC1(N=C=NC2CCN(c3nc(OC[C@@H]4CCCN4C)nc4c3CCN(c3cccc5cccc(C)c35)C4)CC2)CC1] | ++ | + | ++ |
| 81 | 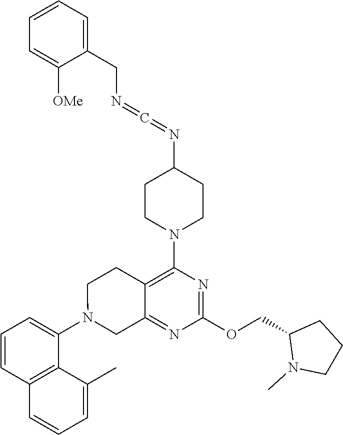 [SMILES: COc1ccccc1CN=C=NC1CCN(c2nc(OC[C@@H]3CCCN3C)nc3c2CCN(c2cccc4cccc(C)c24)C3)CC1] | + | + | + |
| 82 | 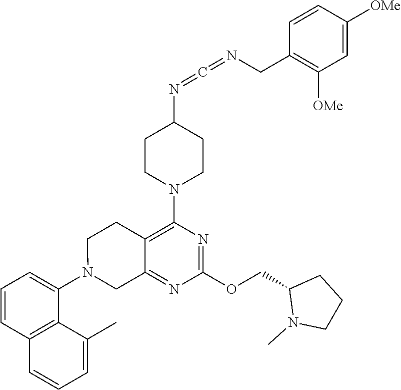 [SMILES: COc1ccc(CN=C=NC2CCN(c3nc(OC[C@@H]4CCCN4C)nc4c3CCN(c3cccc5cccc(C)c35)C4)CC2)c(OC)c1] | + | + | + |
| 83 | 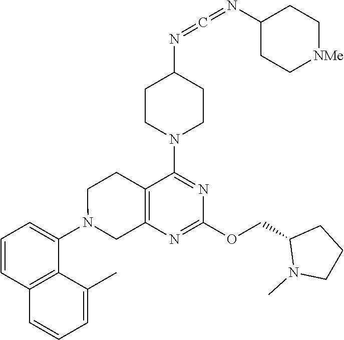 [SMILES: Cc1cccc2cccc(N3CCc4c(nc(OC[C@@H]5CCCN5C)nc4N4CCC(N=C=NC5CCN(C)CC5)CC4)C3)c12] | +++ | ++ | ++ |
| 84 | 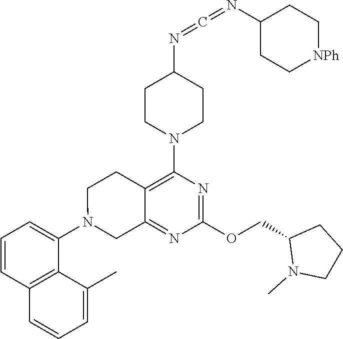 | + | + | + |
| 85 | 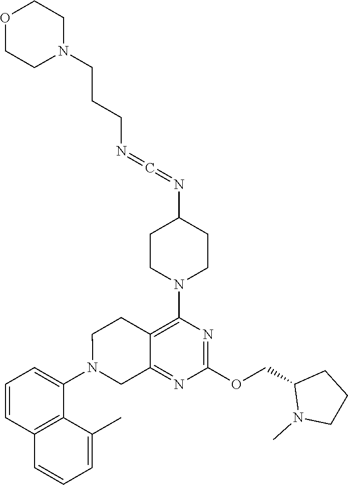 [SMILES: Cc1cccc2cccc(N3CCc4c(nc(OC[C@@H]5CCCN5C)nc4N4CCC(N=C=NCCCN5CCOCC5)CC4)C3)c12] | +++ | ++ | ++ |
| 86 | 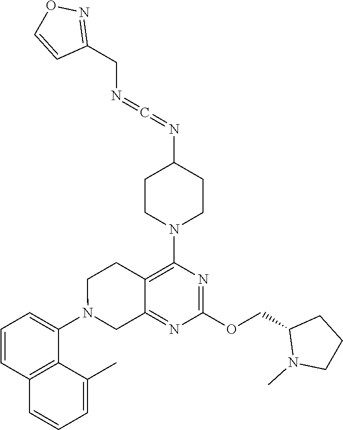 [SMILES: Cc1cccc2cccc(N3CCc4c(nc(OC[C@@H]5CCCN5C)nc4N4CCC(N=C=NCc5ccon5)CC4)C3)c12] | +++ | ++ | ++ |
| 87 | 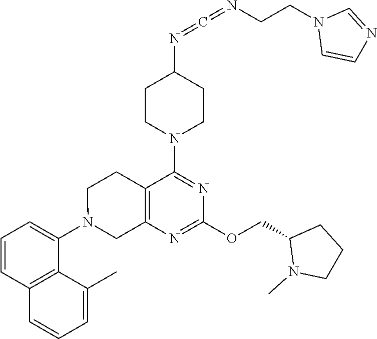 [SMILES: Cc1cccc2cccc(N3CCc4c(nc(OC[C@@H]5CCCN5C)nc4N4CCC(N=C=NCCn5ccnc5)CC4)C3)c12] | +++ | ++ | ++ |
| 88 | 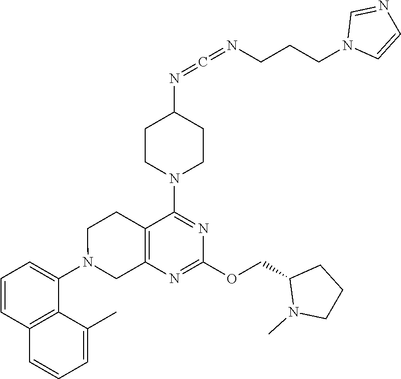 [SMILES: Cc1cccc2cccc(N3CCc4c(nc(OC[C@@H]5CCCN5C)nc4N4CCC(N=C=NCCCn5ccnc5)CC4)C3)c12] | +++ | ++ | ++ |
| 89 | 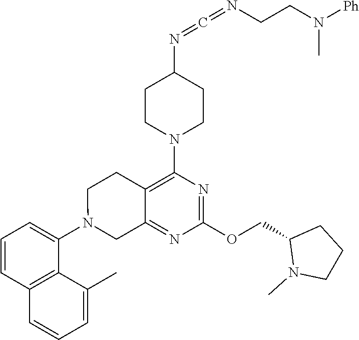 [SMILES: Cc1cccc2cccc(N3CCc4c(nc(OC[C@@H]5CCCN5C)nc4N4CCC(N=C=NCCN(C)c5ccccc5)CC4)C3)c12] | + | + | + |
| 90 | 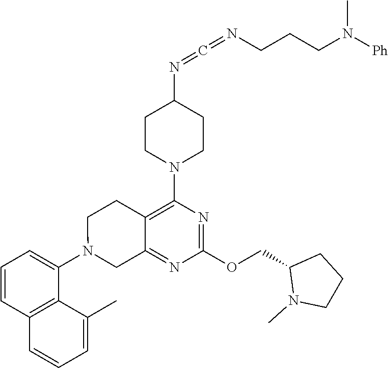 [SMILES: Cc1cccc2cccc(N3CCc4c(nc(OC[C@@H]5CCCN5C)nc4N4CCC(N=C=NCCCN(C)c5ccccc5)CC4)C3)c12] | + | + | + |
| 91 | 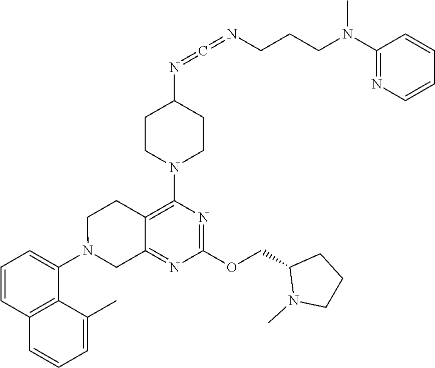 [SMILES: Cc1cccc2cccc(N3CCc4c(nc(OC[C@@H]5CCCN5C)nc4N4CCC(N=C=NCCCN(C)c5ccccn5)CC4)C3)c12] | ++ | + | + |
| 92 | 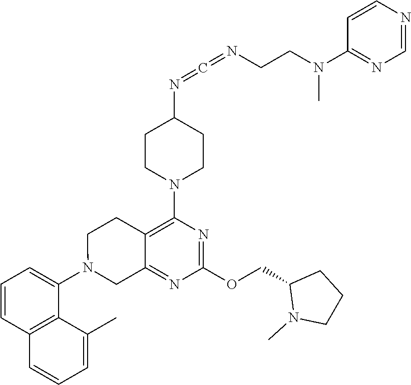 [SMILES: Cc1cccc2cccc(N3CCc4c(nc(OC[C@@H]5CCCN5C)nc4N4CCC(N=C=NCCN(C)c5ccncn5)CC4)C3)c12] | +++ | ++ | ++ |
| 93 | 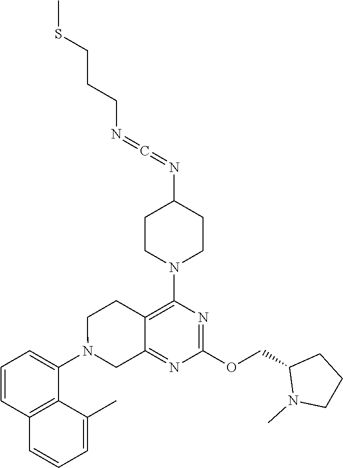 [SMILES: CSCCCN=C=NC1CCN(c2nc(OC[C@@H]3CCCN3C)nc3c2CCN(c2cccc4cccc(C)c24)C3)CC1] | ++ | + | + |
| 94 | 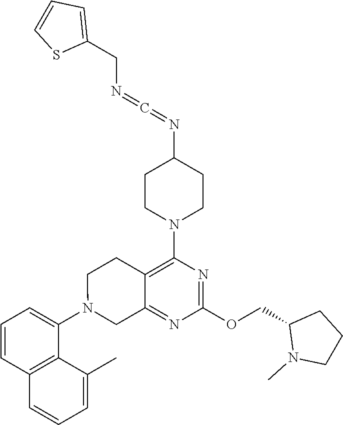 [SMILES: Cc1cccc2cccc(N3CCc4c(nc(OC[C@@H]5CCCN5C)nc4N4CCC(N=C=NCc5cccs5)CC4)C3)c12] | + | + | + |
| 95 | 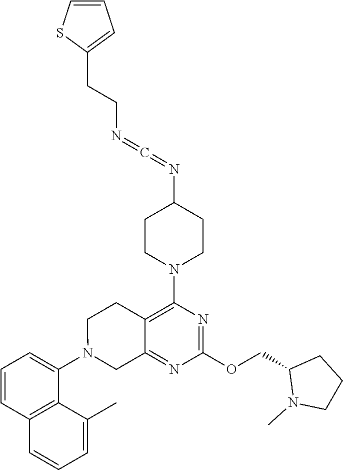 [SMILES: Cc1cccc2cccc(N3CCc4c(nc(OC[C@@H]5CCCN5C)nc4N4CCC(N=C=NCCc5cccs5)CC4)C3)c12] | + | + | + |
| 96 | 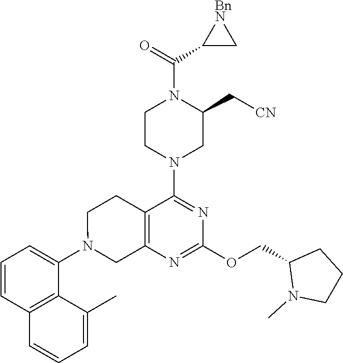 [SMILES: Cc1cccc2cccc(N3CCc4c(nc(OC[C@@H]5CCCN5C)nc4N4CCN(C(=O)[C@H]5CN5Cc5ccccc5)[C@@H](CC#N)C4)C3)c12] | + | + | ND |
| 97 | 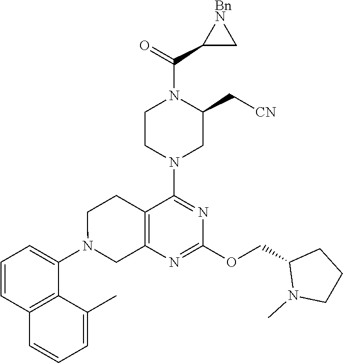 [SMILES: Cc1cccc2cccc(N3CCc4c(nc(OC[C@@H]5CCCN5C)nc4N4CCN(C(=O)[C@@H]5CN5Cc5ccccc5)[C@@H](CC#N)C4)C3)c12] | + | + | ND |
| 98 | 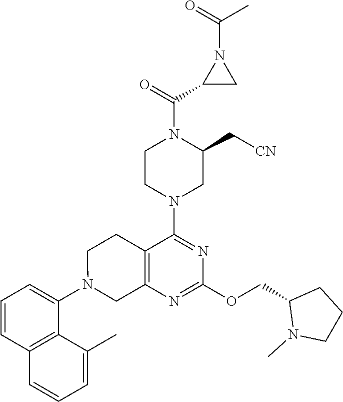 [SMILES: CC(=O)N1C[C@@H]1C(=O)N1CCN(c2nc(OC[C@@H]3CCCN3C)nc3c2CCN(c2cccc4cccc(C)c24)C3)C[C@@H]1CC#N] | + | 0 | ND |
| 99 | 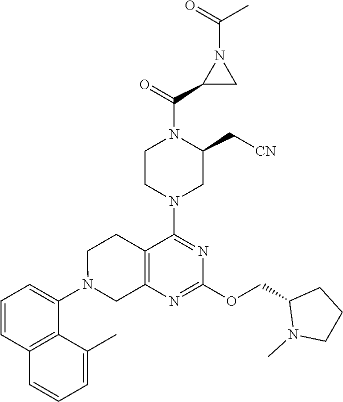 [SMILES: CC(=O)N1C[C@H]1C(=O)N1CCN(c2nc(OC[C@@H]3CCCN3C)nc3c2CCN(c2cccc4cccc(C)c24)C3)C[C@@H]1CC#N] | + | + | ND |
| 100 | 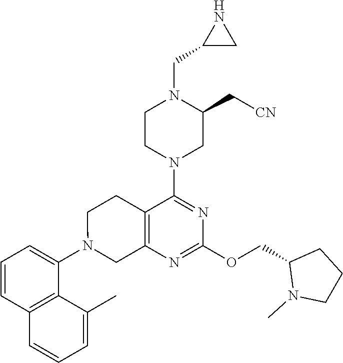 [SMILES: Cc1cccc2cccc(N3CCc4c(nc(OC[C@@H]5CCCN5C)nc4N4CCN(C[C@H]5CN5)[C@@H](CC#N)C4)C3)c12] | + | + | ND |
| 101 | 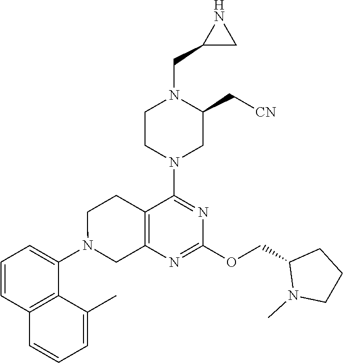 [SMILES: Cc1cccc2cccc(N3CCc4c(nc(OC[C@@H]5CCCN5C)nc4N4CCN(C[C@@H]5CN5)[C@@H](CC#N)C4)C3)c12] | + | + | ND |
| 102 | 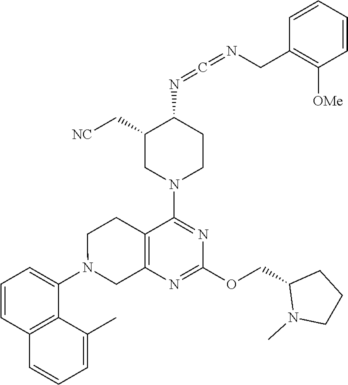 [SMILES: COc1ccccc1CN=C=N[C@@H]1CCN(c2nc(OC[C@@H]3CCCN3C)nc3c2CCN(c2cccc4cccc(C)c24)C3)C[C@@H]1CC#N] | + | + | ND |
| 103 | 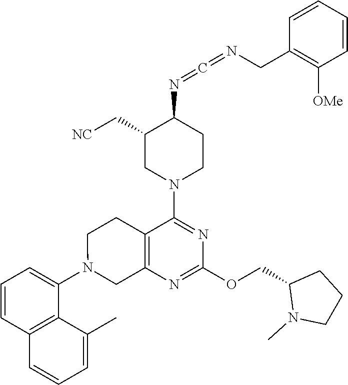 [SMILES: COc1ccccc1CN=C=N[C@H]1CCN(c2nc(OC[C@@H]3CCCN3C)nc3c2CCN(c2cccc4cccc(C)c24)C3)C[C@@H]1CC#N] | + | + | ND |
| 104 | 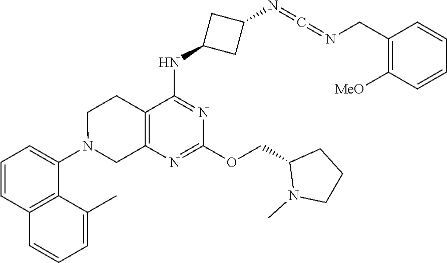 [SMILES: COc1ccccc1CN=C=N[C@H]1C[C@H](Nc2nc(OC[C@@H]3CCCN3C)nc3c2CCN(c2cccc4cccc(C)c24)C3)C1] | + | + | ND |
| 105 | 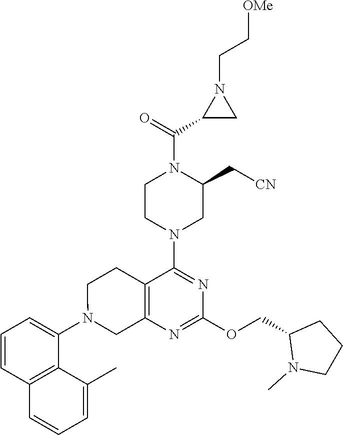 [SMILES: COCCN1C[C@@H]1C(=O)N1CCN(c2nc(OC[C@@H]3CCCN3C)nc3c2CCN(c2cccc4cccc(C)c24)C3)C[C@@H]1CC#N] | ++ | + | ND |
| 106 | 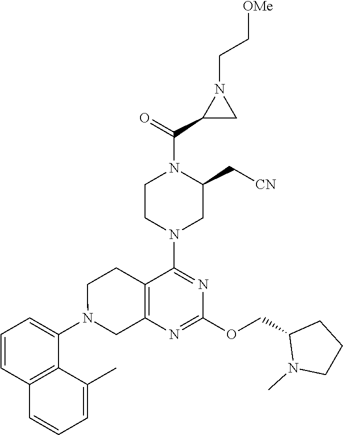 [SMILES: COCCN1C[C@H]1C(=O)N1CCN(c2nc(OC[C@@H]3CCCN3C)nc3c2CCN(c2cccc4cccc(C)c24)C3)C[C@@H]1CC#N] | + | + | ND |
| 107 | 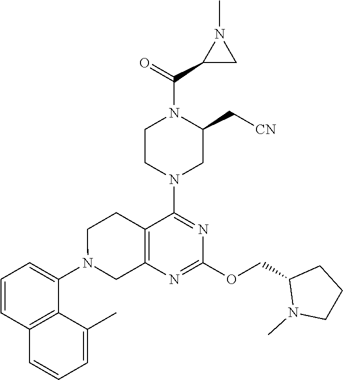 [SMILES: Cc1cccc2cccc(N3CCc4c(nc(OC[C@@H]5CCCN5C)nc4N4CCN(C(=O)[C@@H]5CN5C)[C@@H](CC#N)C4)C3)c12] | + | 0 | ND |
| 108 | 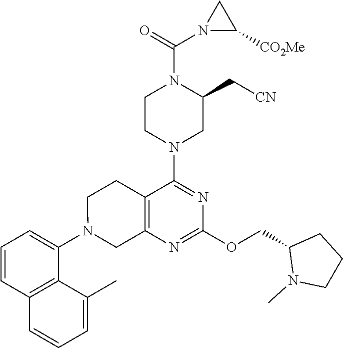 [SMILES: COC(=O)[C@H]1CN1C(=O)N1CCN(c2nc(OC[C@@H]3CCCN3C)nc3c2CCN(c2cccc4cccc(C)c24)C3)C[C@@H]1CC#N] | + | + | ND |
| 109 | 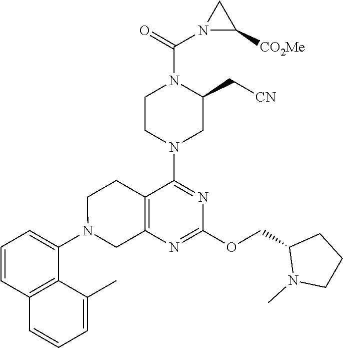 [SMILES: COC(=O)[C@@H]1CN1C(=O)N1CCN(c2nc(OC[C@@H]3CCCN3C)nc3c2CCN(c2cccc4cccc(C)c24)C3)C[C@@H]1CC#N] | + | + | ND |
| 110 | 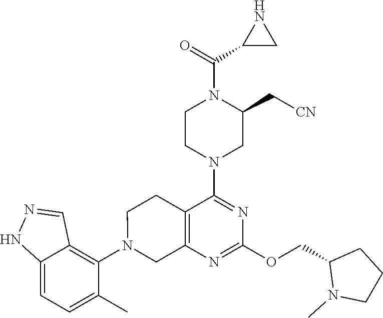 [SMILES: Cc1ccc2[nH]ncc2c1N1CCc2c(nc(OC[C@@H]3CCCN3C)nc2N2CCN(C(=O)[C@H]3CN3)[C@@H](CC#N)C2)C1] | + | + | ND |
| 111 | 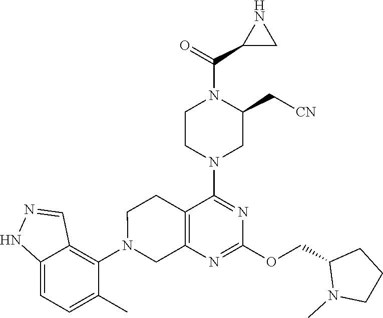 [SMILES: Cc1ccc2[nH]ncc2c1N1CCc2c(nc(OC[C@@H]3CCCN3C)nc2N2CCN(C(=O)[C@@H]3CN3)[C@@H](CC#N)C2)C1] | + | 0 | ND |
| 112 | 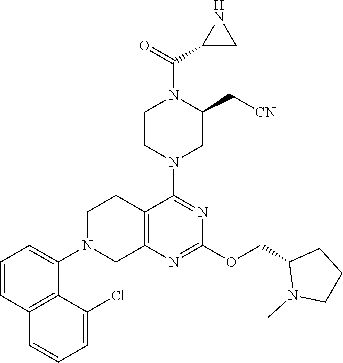 [SMILES: CN1CCC[C@H]1COc1nc2c(c(N3CCN(C(=O)[C@H]4CN4)[C@@H](CC#N)C3)n1)CCN(c1cccc3cccc(Cl)c13)C2] | ++ | 0 | ND |
| 113 | 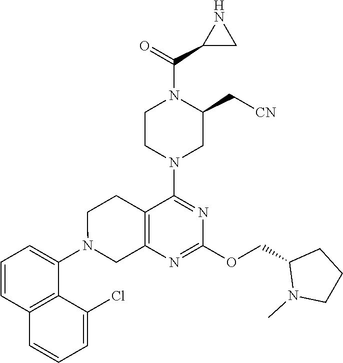 [SMILES: CN1CCC[C@H]1COc1nc2c(c(N3CCN(C(=O)[C@@H]4CN4)[C@@H](CC#N)C3)n1)CCN(c1cccc3cccc(Cl)c13)C2] | + | + | ND |
| 114 | 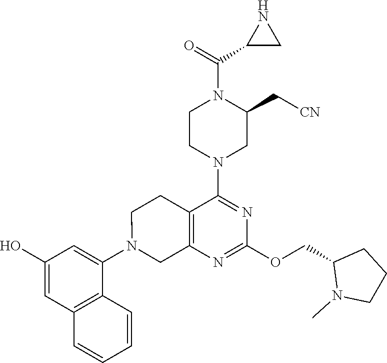 [SMILES: CN1CCC[C@H]1COc1nc2c(c(N3CCN(C(=O)[C@H]4CN4)[C@@H](CC#N)C3)n1)CCN(c1cc(O)cc3ccccc13)C2] | ++ | 0 | ND |
| 115 | 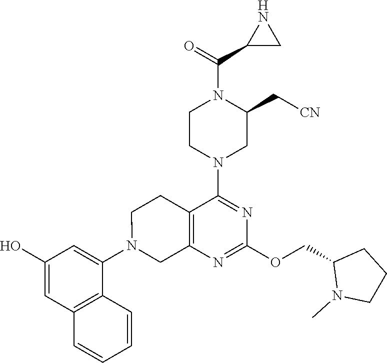 [SMILES: CN1CCC[C@H]1COc1nc2c(c(N3CCN(C(=O)[C@@H]4CN4)[C@@H](CC#N)C3)n1)CCN(c1cc(O)cc3ccccc13)C2] | + | 0 | ND |
| 116 | 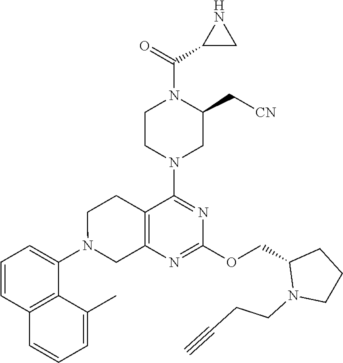 [SMILES: C#CCCN1CCC[C@H]1COc1nc2c(c(N3CCN(C(=O)[C@H]4CN4)[C@@H](CC#N)C3)n1)CCN(c1cccc3cccc(C)c13)C2] | + | + | ND |
| 117 | 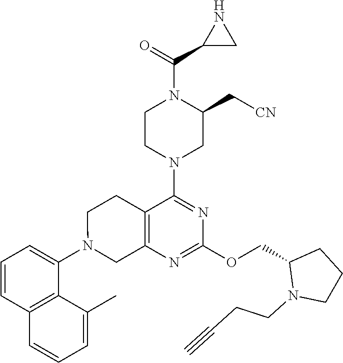 [SMILES: C#CCCN1CCC[C@H]1COc1nc2c(c(N3CCN(C(=O)[C@@H]4CN4)[C@@H](CC#N)C3)n1)CCN(c1cccc3cccc(C)c13)C2] | + | 0 | ND |
| 118 | 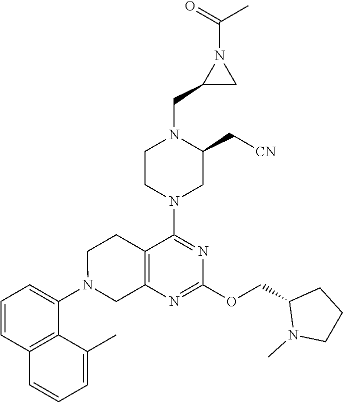 [SMILES: CC(=O)N1C[C@H]1CN1CCN(c2nc(OC[C@@H]3CCCN3C)nc3c2CCN(c2cccc4cccc(C)c24)C3)C[C@@H]1CC#N] | + | + | ND |
| 119 | 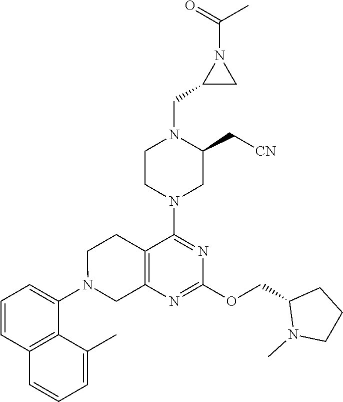 [SMILES: CC(=O)N1C[C@@H]1CN1CCN(c2nc(OC[C@@H]3CCCN3C)nc3c2CCN(c2cccc4cccc(C)c24)C3)C[C@@H]1CC#N] | + | + | ND |
| 120 | 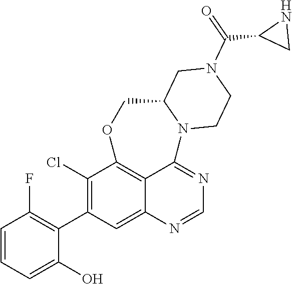 [SMILES: O=C([C@H]1CN1)N1CCN2c3ncnc4cc(-c5c(O)cccc5F)c(Cl)c(c34)OC[C@@H]2C1] | 0 | 0 | ND |
| 121 | 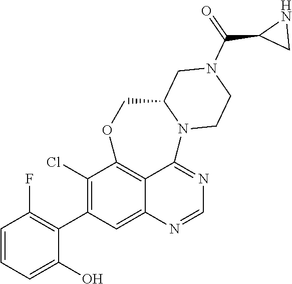 [SMILES: O=C([C@@H]1CN1)N1CCN2c3ncnc4cc(-c5c(O)cccc5F)c(Cl)c(c34)OC[C@@H]2C1] | 0 | 0 | ND |
| 123 | 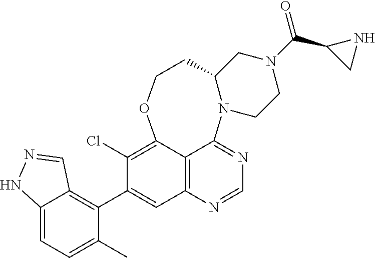 [SMILES: Cc1ccc2[nH]ncc2c1-c1cc2ncnc3c2c(c1Cl)OCC[C@@H]1CN(C(=O)[C@@H]2CN2)CCN31] | 0 | 0 | ND |
| 125 | 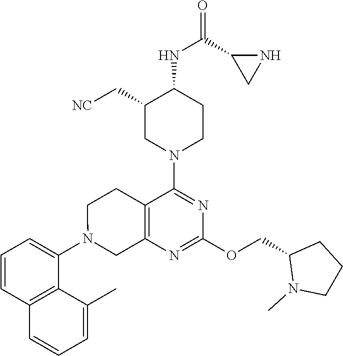 [SMILES: Cc1cccc2cccc(N3CCc4c(nc(OC[C@@H]5CCCN5C)nc4N4CC[C@@H](NC(=O)[C@H]5CN5)[C@@H](CC#N)C4)C3)c12] | + | + | ND |
| 126 | 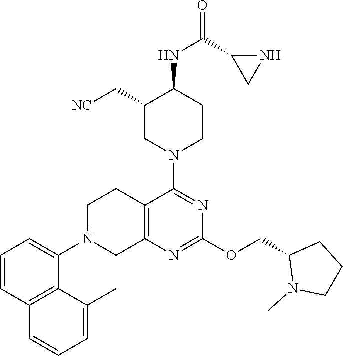 [SMILES: Cc1cccc2cccc(N3CCc4c(nc(OC[C@@H]5CCCN5C)nc4N4CC[C@H](NC(=O)[C@H]5CN5)[C@@H](CC#N)C4)C3)c12] | + | + | ND |
| 127 | 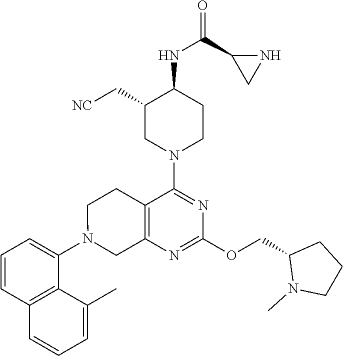 [SMILES: Cc1cccc2cccc(N3CCc4c(nc(OC[C@@H]5CCCN5C)nc4N4CC[C@H](NC(=O)[C@@H]5CN5)[C@@H](CC#N)C4)C3)c12] | + | + | ND |
| 128 | 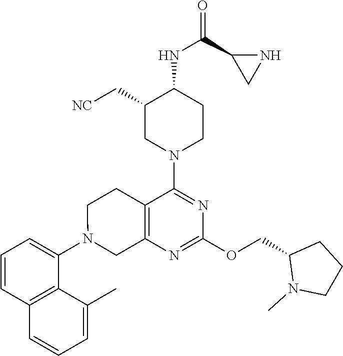 [SMILES: Cc1cccc2cccc(N3CCc4c(nc(OC[C@@H]5CCCN5C)nc4N4CC[C@@H](NC(=O)[C@@H]5CN5)[C@@H](CC#N)C4)C3)c12] | + | + | ND |
| 129 | 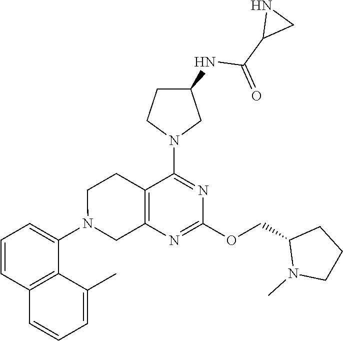 [SMILES: Cc1cccc2cccc(N3CCc4c(nc(OC[C@@H]5CCCN5C)nc4N4CC[C@@H](NC(=O)C5CN5)C4)C3)c12] | + | 0 | ND |
| 130 | 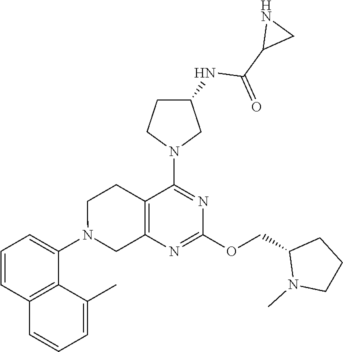 [SMILES: Cc1cccc2cccc(N3CCc4c(nc(OC[C@@H]5CCCN5C)nc4N4CC[C@H](NC(=O)C5CN5)C4)C3)c12] | + | 0 | ND |
| 131 | 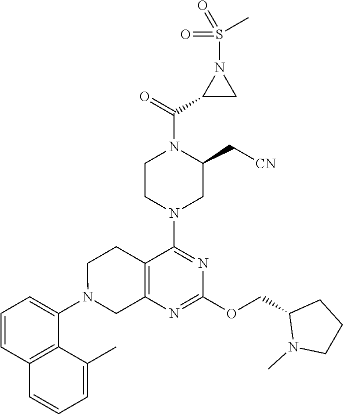 [SMILES: Cc1cccc2cccc(N3CCc4c(nc(OC[C@@H]5CCCN5C)nc4N4CCN(C(=O)[C@H]5CN5S(C)(=O)=O)[C@@H](CC#N)C4)C3)c12] | + | + | ND |
| 132 | 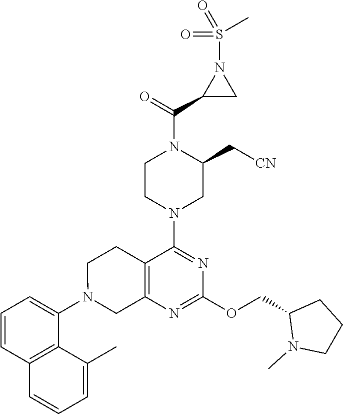 [SMILES: Cc1cccc2cccc(N3CCc4c(nc(OC[C@@H]5CCCN5C)nc4N4CCN(C(=O)[C@@H]5CN5S(C)(=O)=O)[C@@H](CC#N)C4)C3)c12] | + | + | ND |
| 134 | 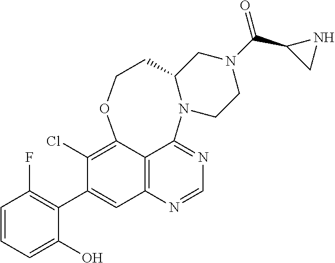 [SMILES: O=C([C@@H]1CN1)N1CCN2c3ncnc4cc(-c5c(O)cccc5F)c(Cl)c(c34)OCC[C@@H]2C1] | + | + | ND |
| 135 | 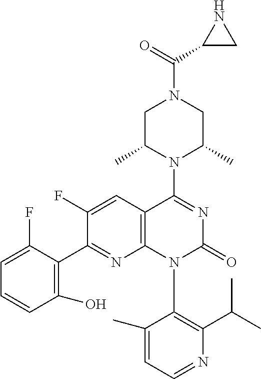 [SMILES: Cc1ccnc(C(C)C)c1-n1c(=O)nc(N2[C@H](C)CN(C(=O)[C@H]3CN3)C[C@@H]2C)c2cc(F)c(-c3c(O)cccc3F)nc21] | 0 | 0 | ND |
| 136 | 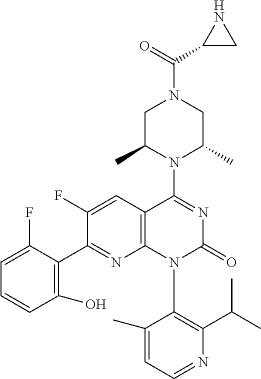 [SMILES: Cc1ccnc(C(C)C)c1-n1c(=O)nc(N2[C@@H](C)CN(C(=O)[C@H]3CN3)C[C@@H]2C)c2cc(F)c(-c3c(O)cccc3F)nc21] | + | + | ND |
| 137 | 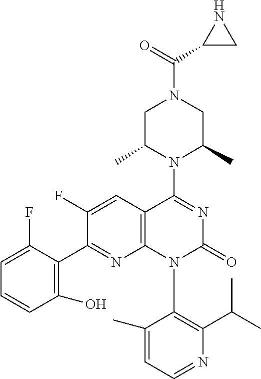 [SMILES: Cc1ccnc(C(C)C)c1-n1c(=O)nc(N2[C@H](C)CN(C(=O)[C@H]3CN3)C[C@H]2C)c2cc(F)c(-c3c(O)cccc3F)nc21] | 0 | 0 | ND |
| 138 | 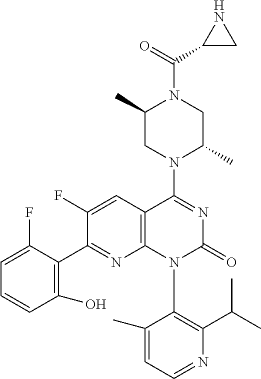 [SMILES: Cc1ccnc(C(C)C)c1-n1c(=O)nc(N2C[C@@H](C)N(C(=O)[C@H]3CN3)C[C@@H]2C)c2cc(F)c(-c3c(O)cccc3F)nc21] | 0 | 0 | ND |
| 139 | 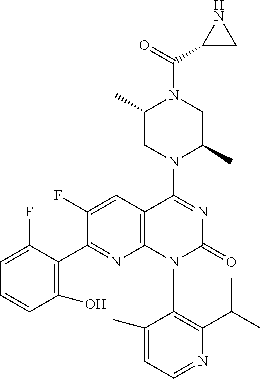 [SMILES: Cc1ccnc(C(C)C)c1-n1c(=O)nc(N2C[C@H](C)N(C(=O)[C@H]3CN3)C[C@H]2C)c2cc(F)c(-c3c(O)cccc3F)nc21] | 0 | 0 | ND |
| 140 | 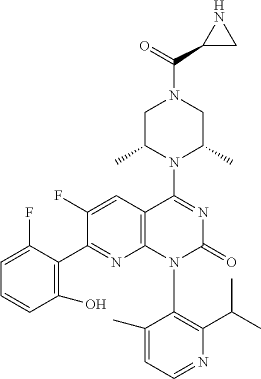 [SMILES: Cc1ccnc(C(C)C)c1-n1c(=O)nc(N2[C@H](C)CN(C(=O)[C@@H]3CN3)C[C@@H]2C)c2cc(F)c(-c3c(O)cccc3F)nc21] | 0 | 0 | ND |
| 142 | 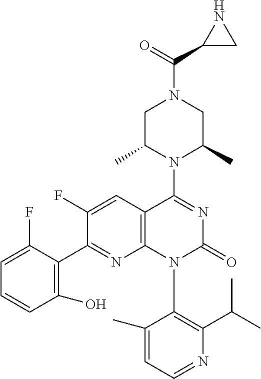 [SMILES: Cc1ccnc(C(C)C)c1-n1c(=O)nc(N2[C@H](C)CN(C(=O)[C@@H]3CN3)C[C@H]2C)c2cc(F)c(-c3c(O)cccc3F)nc21] | + | + | ND |
| 143 | 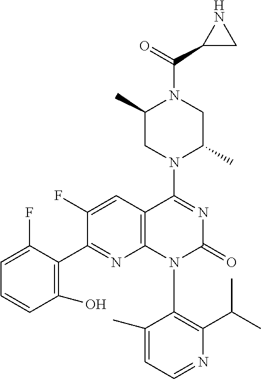 [SMILES: Cc1ccnc(C(C)C)c1-n1c(=O)nc(N2C[C@@H](C)N(C(=O)[C@@H]3CN3)C[C@@H]2C)c2cc(F)c(-c3c(O)cccc3F)nc21] | 0 | 0 | ND |
| 144 | 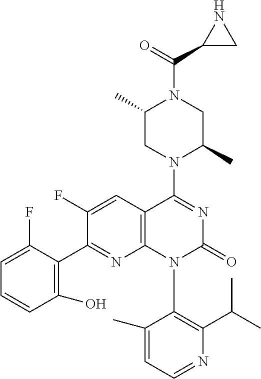 [SMILES: Cc1ccnc(C(C)C)c1-n1c(=O)nc(N2C[C@H](C)N(C(=O)[C@@H]3CN3)C[C@H]2C)c2cc(F)c(-c3c(O)cccc3F)nc21] | 0 | 0 | ND |
| 145 | 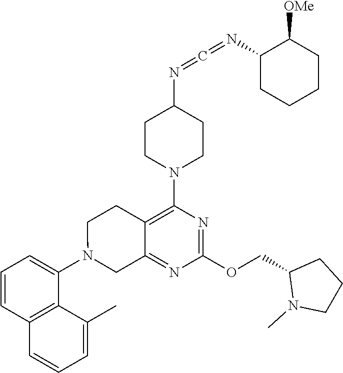 [SMILES: CO[C@H]1CCCC[C@@H]1N=C=NC1CCN(c2nc(OC[C@@H]3CCCN3C)nc3c2CCN(c2cccc4cccc(C)c24)C3)CC1] | + | + | ND |
| 146 | 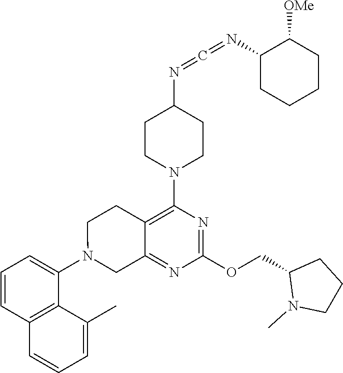 [SMILES: CO[C@@H]1CCCC[C@@H]1N=C=NC1CCN(c2nc(OC[C@@H]3CCCN3C)nc3c2CCN(c2cccc4cccc(C)c24)C3)CC1] | + | + | ND |
| 147 | 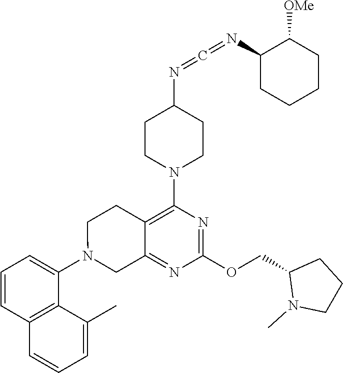 [SMILES: CO[C@@H]1CCCC[C@H]1N=C=NC1CCN(c2nc(OC[C@@H]3CCCN3C)nc3c2CCN(c2cccc4cccc(C)c24)C3)CC1] | + | + | ND |
| 148 | 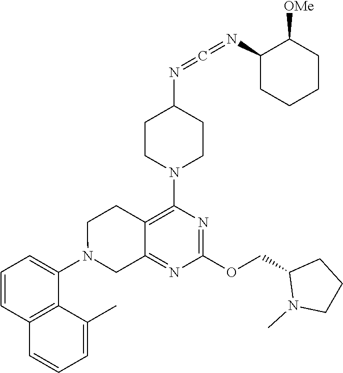 [SMILES: CO[C@H]1CCCC[C@H]1N=C=NC1CCN(c2nc(OC[C@@H]3CCCN3C)nc3c2CCN(c2cccc4cccc(C)c24)C3)CC1] | + | + | ND |
| 149 | 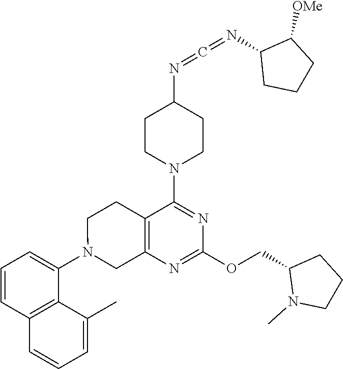 [SMILES: CO[C@@H]1CCC[C@@H]1N=C=NC1CCN(c2nc(OC[C@@H]3CCCN3C)nc3c2CCN(c2cccc4cccc(C)c24)C3)CC1] | ++ | + | ND |
| 150 | 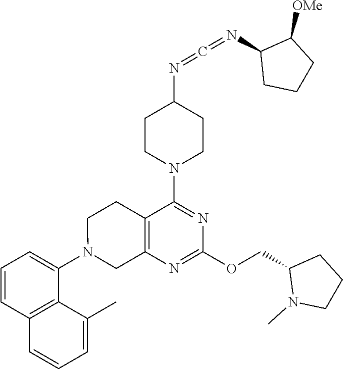 [SMILES: CO[C@H]1CCC[C@H]1N=C=NC1CCN(c2nc(OC[C@@H]3CCCN3C)nc3c2CCN(c2cccc4cccc(C)c24)C3)CC1] | + | + | ND |
| 151 | 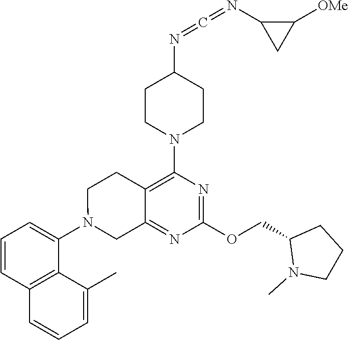 [SMILES: COC1CC1N=C=NC1CCN(c2nc(OC[C@@H]3CCCN3C)nc3c2CCN(c2cccc4cccc(C)c24)C3)CC1] | +++ | + | ND |
| 152 | 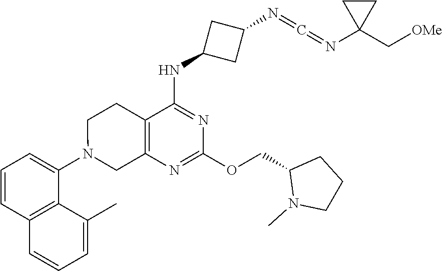 [SMILES: COCC1(N=C=N[C@H]2C[C@H](Nc3nc(OC[C@@H]4CCCN4C)nc4c3CCN(c3cccc5cccc(C)c35)C4)C2)CC1] | +++ | + | ND |
| 153 | 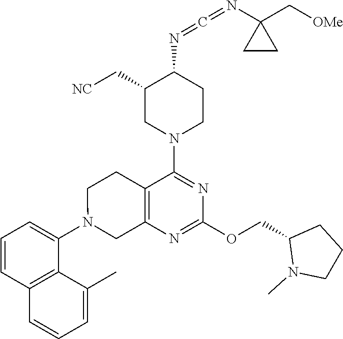 [SMILES: COCC1(N=C=N[C@@H]2CCN(c3nc(OC[C@@H]4CCCN4C)nc4c3CCN(c3cccc5cccc(C)c35)C4)C[C@@H]2CC#N)CC1] | ++ | + | ND |

Key:
G12D ranges:

| symbol | Range (% x-linking) |
|---|---|
| 0 | No observed x-linking |
| + | 0 < x ≤ 20 |
| ++ | 20 < x ≤ 40 |
| +++ | 40 < x ≤ 100 |

WT ranges:

| symbol | Range (% x-linking) |
|---|---|
| 0 | No observed x-linking |
| + | 0 < x ≤ 20 |
| ++ | 20 < x ≤ 100 |

G12S ranges:

| symbol | Range (% x-linking) |
|---|---|
| 0 | No observed x-linking |
| + | 0 < x ≤ 10 |
| ++ | 10 < x ≤ 100 |

ND = Not Determined, for each Ras protein type

### Example 220—Synthesis of Exemplary Compounds in Table 2f and Other Exemplary Compounds

The compounds in Table 2f can be prepared according to reaction scheme 1, using carboxylic acids such as intermediates 1-41 (and analogous intermediates) and an amine similar to compound 1. Some intermediates yield a product that may be deprotected. Deprotection methods are known in the art, some of which are described below.

Intermediates 1, 2, 5, 6, 9, 10, 13, 14, 23, and 24 can be reacted with an amine similar to compound 1 in reaction scheme 1 to provide products that contain a sulfinamide. Sulfinamide can be removed under acidic conditions (e.g., HI in THF at 0° C.).

Intermediates 7 and 8 can be reacted with an amine similar to compound 1 in reaction scheme 1 to provide products that contain a trityl group. A trityl group can be removed under acidic conditions (e.g., TFA).

Intermediates 19, 20, 21, 22, 27, and 28 can be reacted with an amine similar to compound 1 in reaction scheme 1 to provide products that contain a benzyl group. A benzyl group can be removed under hydrogenolysis conditions (e.g., H₂ in the presence of a catalyst, e.g., Pd/C).

Intermediates 25 and 26 can be reacted with an amine similar to compound 1 in reaction scheme 1 to provide products that contain a para-methoxybenzyl group. A para-methoxybenzyl group can be removed under oxidative conditions.

Intermediate 29 can be reacted with an amine similar to compound 1 in reaction scheme 1 to provide a product that contains a Cbz group. A Cbz group can be removed under hydrogenolysis conditions (e.g., H₂ in the presence of a catalyst, e.g., Pd/C).

Intermediates 32 and 33 can be reacted with an amine similar to compound 1 in reaction scheme 1 to provide products that contain a TBDPS group. A TBDPS group can be removed using TBAF.

Intermediates that can be used may be derived from other intermediates. For example, intermediates 19, 20, 21, and 22 are derived from intermediates 15, 16, 17, and 18 respectively. Intermediates 19, 20, 21, and 22 can be reacted with an amine similar to compound 1 in reaction scheme 1 to provide products that contain a benzhydryl group. A benzhydryl group may be removed under hydrogenolysis conditions or acidic conditions.

In a similar fashion as described above in this Example, persons of skill in the art will be able to incorporate intermediates 1-41 at comparable amine positions of other compounds disclosed herein, deprotecting as appropriate, to arrive at a compound of the present invention.

Those skilled in the art will recognize or be able to ascertain, using no more than routine experimentation, many equivalents to the specific embodiments in accordance with the invention described herein. The scope of the present invention is not intended to be limited to the foregoing description, but rather is as set forth in the appended claims. Moreover, it is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A compound having the structure of Formula I:
A-L-B Formula I
wherein A is a Ras binding moiety;
L is a linker; and
B is a selective cross-linking group,
or a pharmaceutically acceptable salt thereof,
wherein, upon contacting the compound, or a pharmaceutically acceptable salt thereof, with a sample containing a Ras protein, at least 20% of the Ras protein in the sample covalently reacts with the compound, or a pharmaceutically acceptable salt thereof, to form a conjugate.

2. The compound, or a pharmaceutically acceptable salt thereof, of claim 1, wherein the Ras protein in the sample is a mutant Ras protein.

3. The compound, or a pharmaceutically acceptable salt thereof, of claim 1 or 2, wherein the Ras binding moiety is a K-Ras binding moiety and the Ras protein in the sample is a K-Ras protein.

4. The compound, or a pharmaceutically acceptable salt thereof, of claim 3, wherein the K-Ras binding moiety interacts with a residue of a K-Ras Switch-II binding pocket of the K-Ras protein.

5. The compound, or a pharmaceutically acceptable salt thereof, of claim 4, wherein the residue of a K-Ras Switch-II binding pocket is a residue of the K-Ras protein corresponding to V7, V8, V9, G10, A11, D12, K16, P34, T58, A59, G60, Q61, E62, E63, Y64, S65, R68, D69, Y71, M72, F78, I92, H95, Y96, Q99, I100, R102, or V103 of human wild-type K-Ras (SEQ ID NO: 1).

6. The compound, or a pharmaceutically acceptable salt thereof, of any one of claims 3 to 5, wherein the K-Ras binding moiety is the structure of any one of Formulas II-V.

7. The compound, or a pharmaceutically acceptable salt thereof, of claim 6, wherein the K-Ras binding moiety is the structure of Formula II: 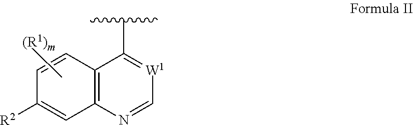
wherein m is 0, 1, 2, or 3;
W¹ is N or C, wherein C is optionally attached to the linker via an optionally substituted C₁-C₃ alkylene bridge or an optionally substituted C₁-C₃ heteroalkylene bridge;
each R¹ is, independently, CN, halo, hydroxy, optionally substituted C₁-C₆ alkyl, or optionally substituted C₁-C₆ heteroalkyl, or
R¹ is attached to the linker via a C₁-C₃ alkylene bridge or C₁-C₃ heteroalkylene bridge; and
R² is optionally substituted C₆-C₁₀ aryl or optionally substituted C₂-C₉ heteroaryl.

8. The compound, or a pharmaceutically acceptable salt thereof, of claim 6, wherein the K-Ras binding moiety is the structure of Formula III: 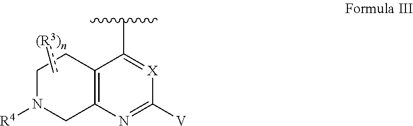
wherein n is 0, 1, 2, 3, 4, 5, or 6; [symbol]
represents a single bond or a double bond;
X is N or CR′, wherein R′ is hydrogen, or R′ is attached to the linker via an optionally substituted C₁-C₃alkylene bridge, or optionally substituted C₁-C₃ heteroalkylene bridge;
V is CHR⁵, CR⁵R⁵, OR⁵, NHR⁵, or NR^{5a}R^{5b};
each R³ is, independently, 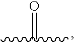 [SMILES: *=O]
optionally substituted C₁-C₆ alkyl, or optionally substituted C₁-C₆ heteroalkyl, or
R³ is attached to the linker via an optionally substituted C₁-C₃ alkylene bridge or optionally substituted C₁-C₃ heteroalkylene bridge;
R⁴ is optionally substituted C₆-C₁₀ aryl or optionally substituted C₂-C₉ heteroaryl;
each R⁵ is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl; and
each of R^{5a} and R^{5b} is, independently, optionally substituted C₁-C₆ alkyl, optionally substituted C₁-C₆ heteroalkyl, optionally substituted —C₁-C₆ alkyl-C₂-C₉ heteroaryl or optionally substituted —C₁-C₆ alkyl-C₂-C₉ heterocyclyl, or
R^{5a} and R^{5b}, together with the nitrogen atom to which each is attached, combine to form optionally substituted C₂-C₉ heterocyclyl;
provided that when R′ is attached to the linker via an optionally substituted C₁-C₃ alkylene bridge or optionally substituted C₁-C₃ heteroalkylene bridge, then R³ is not attached to the linker via an optionally substituted C₁-C₃ alkylene bridge or optionally substituted C₁-C₃ heteroalkylene bridge, and
further provided that when R³ is attached to the linker via an optionally substituted C₁-C₃ alkylene bridge or optionally substituted C₁-C₃ heteroalkylene bridge, R′ not is attached to the linker via an optionally substituted C₁-C₃ alkylene bridge or optionally substituted C₁-C₃ heteroalkylene bridge.

9. The compound, or a pharmaceutically acceptable salt thereof, of claim 6, wherein the K-Ras binding moiety is the structure of Formula IV: 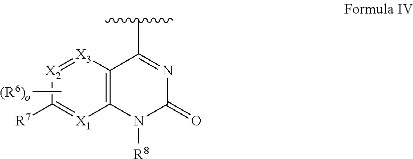
wherein o is 0, 1, or 2;
X¹, X² and X³ are each independently N, CH, or CR⁶;
each R⁶ is, independently, halo, CN, hydroxy, optionally substituted C₁-C₆ alkyl, or optionally substituted C₁-C₆ heteroalkyl, or
R⁶ is attached to the linker via a C₁-C₃ alkyl bridge or C₁-C₃ heteroalkyl bridge; and
R⁷ and R⁸ are, independently, optionally substituted C₆-C₁₀ aryl or optionally substituted C₂-C₉ heteroaryl.

10. The compound, or a pharmaceutically acceptable salt thereof, of claim 9, wherein only one of X¹, X² and X³ is N.

11. The compound, or a pharmaceutically acceptable salt thereof, of claim 6, wherein the K-Ras binding moiety is the structure of Formula V: 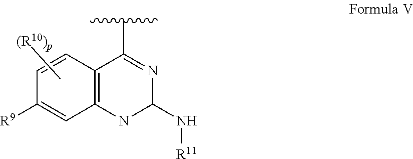
wherein p is 0, 1, 2, or 3;
W⁴ is NH or O;
R⁹ is optionally substituted C₆-C₁₀ aryl or optionally substituted C₂-C₉ heteroaryl;
each R¹⁰ is, independently, halo, CN, hydroxy, optionally substituted C₁-C₆ alkyl, or optionally substituted C₁-C₆ heteroalkyl, or R¹⁰ is attached to the linker via a C₁-C₃ alkylene bridge or C₁-C₃ heteroalkylene bridge; and
R¹¹ is optionally substituted —C₁-C₆ alkyl-C₂-C₉ heteroaryl, optionally substituted —C₁-C₆ alkyl-C₂-C₉ heterocyclyl, optionally substituted C₂-C₉ heteroaryl, or optionally substituted C₂-C₉ heterocyclyl.

12. The compound, or a pharmaceutically acceptable salt thereof, of any one of claims 1 to 11, wherein the linker positions a reactive atom of B about 5 to about 11 angstroms from the nearest atom of A.

13. The compound, or a pharmaceutically acceptable salt thereof, of any one of claims 1 to 12, wherein the linker is the structure of Formula VI:
A¹-(B¹)ₐ-(C¹)_{b}-(B²)_{c}-(D)-(B³)_{d}-(C²)ₑ-(B⁴)_{f}-A² Formula VI
wherein A¹ is a bond between the linker and the Ras binding moiety;
A² is a bond between the selective cross-linking group and the linker;
B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl;
C¹ and C² are each, independently, selected from carbonyl, thiocarbonyl, sulphonyl, or phosphoryl;
a, b, c, d, e, and f are each, independently, 0 or 1; and
D is optionally substituted C₁₋₁₀ alkylene, optionally substituted C₂₋₁₀ alkenylene, optionally substituted C₂₋₁₀ alkynylene, optionally substituted C₂₋₆ heterocyclylene, optionally substituted C₂₋₆ heteroarylene, optionally substituted C₃₋₈ cycloalkylene, optionally substituted C₆₋₁₂ arylene, optionally substituted C₂-C₁₀ polyethylene glycol, or optionally substituted C₁₋₁₀ heteroalkylene, or a chemical bond linking A¹-(B¹)ₐ-(C¹)_{b}-(B²)_{c}- to -(B³)_{d}-(C²)ₑ-(B⁴)_{f}-A².

14. The compound, or a pharmaceutically acceptable salt thereof, of any one of claims 1 to 13, wherein the linker comprises a 3 to 8-membered heterocyclyl group.

15. The compound, or a pharmaceutically acceptable salt thereof, of any one of claims 1 to 13, wherein the linker is acyclic.

16. The compound, or a pharmaceutically acceptable salt thereof, of any one of claims 1 to 15, wherein the selective cross-linking group is a C—O bond forming selective cross-linking group.

17. The compound, or a pharmaceutically acceptable salt thereof, of claim 16, having the structure of Formula XXIV: 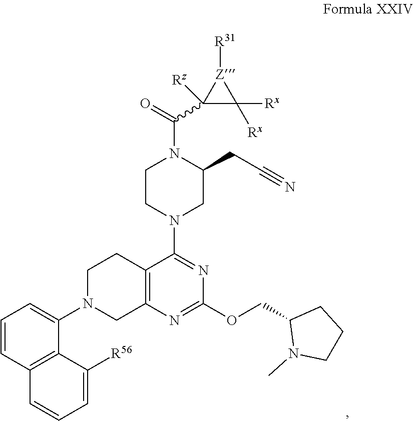
wherein R³¹ is absent, hydrogen, C(O)CH₃, SO₂CH₃, optionally substituted C₁-C₆ alkyl, optionally substituted C₁-C₆ heteroalkyl, optionally substituted C₆-C₁₀ aryl, optionally substituted C₁-C₃ alkyl-C₆-C₁₀ aryl, optionally substituted C₂-C₉ heterocyclyl, or optionally substituted C₁-C₃ alkyl-C₂-C₉ heterocyclyl;
R⁵⁶ is CH₃ or Cl;
R^{z} is hydrogen, optionally substituted C₁-C₃ alkyl;
each R^{x} is, independently, hydrogen, CO₂CH₃, optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, optionally substituted C₂-C₆ alkenyl, or optionally substituted C₂-C₆ alkynyl; and
Z′″ is N or O.

18. The compound, or a pharmaceutically acceptable salt thereof, of claim 16 or 17, having the structure of Formula XIII: 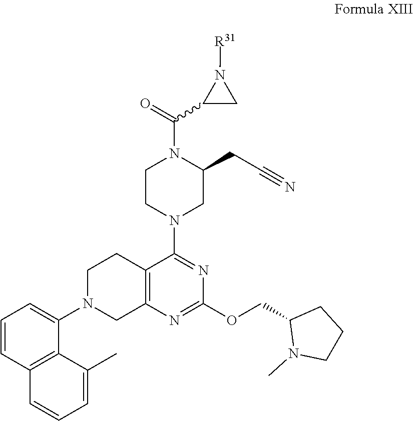
wherein R³¹ is hydrogen, CH₃, C(O)CH₃, SO₂CH₃, CH₂—C₆H₅, or CH₂CH₂OCH₃.

19. The compound, or a pharmaceutically acceptable salt thereof, of claim 1, having the structure of Formula XX or XXI: 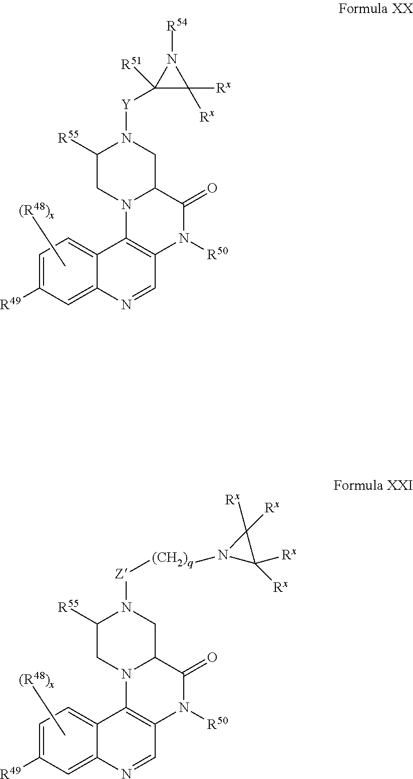
wherein Y is C(O), C(S), SO₂, or optionally substituted C₁-C₆ alkyl;
Z′ is C(O) or SO₂;
q is 0, 1 or 2;
x is 0, 1, 2 or 3;
each R^{X} is, independently, hydrogen, CN, C(O)R^{y}, CO₂R^{y}, C(O)NR^{y}R^{y} optionally substituted C₁-C₆ alkyl, optionally substituted C₁-C₆ heteroalkyl, optionally substituted C₃-C₁₀ cycloalkyl, optionally substituted C₆-C₁₀ aryl, optionally substituted C₂-C₉ heterocyclyl, or optionally substituted C₂-C₉ heteroaryl;
each R^{y} is, independently, hydrogen, optionally substituted C₁-C₆ alkyl, optionally substituted C₆-C₁₀ aryl, optionally substituted C₂-C₉ heterocyclyl, or optionally substituted C₂-C₉ heteroaryl;
each R⁴⁸ is, independently, CN, halo, hydroxy, optionally substituted C₁-C₆ alkyl, or optionally substituted C₁-C₆ heteroalkyl, or
R⁴⁹ is optionally substituted C₆-C₁₀ aryl or optionally substituted C₂-C₉ heteroaryl;
R⁵⁰ is hydrogen or C₁-C₆ alkyl;
R⁵¹ is hydrogen, CN or C₁-C₆ alkyl;
R⁵⁴ is hydrogen, —C(O)R³², —SO₂R³³, optionally substituted C₁-C₆ alkyl, optionally substituted C₁-C₆ heteroalkyl, optionally substituted C₆-C₁₀ aryl, optionally substituted C₂-C₉ heterocyclyl, or optionally substituted C₂-C₉ heteroaryl; and
R⁵⁵ is hydrogen or optionally substituted C₁-C₆ alkyl.

20. The compound, or a pharmaceutically acceptable salt thereof, of claim 19 having the structure of Formula XXII or Formula XXIII: 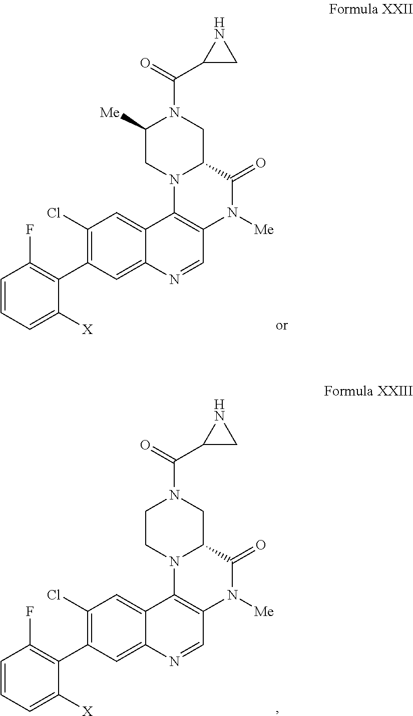
wherein X is hydrogen or hydroxy.

21. A compound, or a pharmaceutically acceptable salt thereof, having the structure of any one of Examples 63-95 in Table 2b.

22. A compound, or a pharmaceutically acceptable salt thereof, having the structure of any one of Examples 96-104 in Table 2c.

23. A compound, or a pharmaceutically acceptable salt thereof, having the structure of any one of Examples 105-180 in Table 2d.

24. A compound, or a pharmaceutically acceptable salt thereof, having the structure of any one of Examples 181-216 in Table 2e.

25. A compound, or a pharmaceutically acceptable salt thereof, having the structure of any one of Examples 217-300 in Table 2f.

26. A pharmaceutical composition comprising a compound, or a pharmaceutically acceptable salt thereof, of any one of claims 1 to 25 and a pharmaceutically acceptable excipient.

27. A conjugate, or salt thereof, comprising a Ras protein covalently bound to a selective cross-linking group, which selective cross-linking group is bound to a Ras binding moiety through a linker, wherein the selective cross-linking group is a carbodiimide, an aminooxazoline, a chloroethyl urea, an aziridine, a trifluoromethyl ketone, a boronic acid, a boronic ester, N-ethoxycarbonyl-2-ethoxy-1,2-dihydroquinoline (EEDQ), an iso-EEDQ or other EEDQ derivative, an epoxide, an oxazolium, or a glycal.

28. A method of producing a conjugate comprising contacting a Ras protein with a compound, or a pharmaceutically acceptable salt thereof, of any one of claims 1 to 25 or a pharmaceutical composition of claim 26 under conditions sufficient for the compound to react covalently with the Ras protein.

29. A conjugate produced by the method of claim 28.

30. A method of treating cancer in a subject in need thereof, the method comprising administering to the subject a therapeutically effective amount of a compound, or a pharmaceutically acceptable salt thereof, of any one of claims 1 to 25 or a pharmaceutical composition of claim 26.

31. A method of treating a Ras protein-related disorder in a subject in need thereof, the method comprising administering to the subject a therapeutically effective amount of a compound, or a pharmaceutically acceptable salt thereof, of any one of claims 1 to 25 or a pharmaceutical composition of claim 26.

32. A method of inhibiting a Ras protein in a cell, the method comprising contacting the cell with an effective amount of a compound, or a pharmaceutically acceptable salt thereof, of any one of claims 1 to 25 or a pharmaceutical composition of claim 26.

33. The method of claim 32, wherein the cell is a cancer cell.

34. The method or use of any one of claims 30 to 33, wherein the method further comprises administering an additional anticancer therapy.


SEQUENCE LISTING (3 sequences)

SEQ ID NO: 1 - protein, 189 aa, Homo sapiens [sequence omitted]

SEQ ID NO: 2 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM